(19) United States
(12) Patent Application Publication
(10) Pub. No.: US 2024/0025856 A1
(43) Pub. Date: Jan. 25, 2024

(54) 1,2,3,4-TETRAHYDROQUINOLINE DERIVATIVES AS INHIBITORS OF THE YAP/TAZ-TEAD ACTIVATION FOR TREATING CANCER

(71) Applicants: KATHOLIEKE UNIVERSITEIT LEUVEN, Vlaams-Brabant Leuven (BE); SpringWorks Therapeutics, Inc., Stamford, CT (US); VIB VZW, Gent (BE)

(72) Inventors: Arnaud MARCHAND, Vlaams-Brabant, Leuven (BE); Aurélie CANDI, (US); Bart VANDERHOYDONCK, (US); Matthias VERSELE, (US); Stephen L. GWALTNEY, II, Stamford, CT (US)

(21) Appl. No.: 18/247,007

(22) PCT Filed: Sep. 30, 2021
(86) PCT No.: PCT/US2021/053034

§ 371 Date: Mar. 28, 2023

(30) Foreign Application Priority Data
Sep. 30, 2020 (EP) 20199435.7

(51) Int. Cl. C07D 215/38; C07D 401/04; C07D 417/04; C07D 401/06; C07D 471/04; C07D 417/06; C07D 215/12; C07D 413/04

(52) U.S. Cl. CPC C07D 215/38; C07D 401/04; C07D 417/04; C07D 401/06; C07D 471/04; C07D 417/06; C07D 215/12; C07D 413/04

(57) ABSTRACT

The present invention relates to novel compounds of formula (Ia), to said compounds for use as a medicine, more in particular for the prevention or treatment of diseases mediated by activity of YAP/TAZ-TEAD transcription, yet more in particular for the prevention or treatment of cancer or fibrosis. The present invention also relates to a method for the prevention or treatment of said diseases comprising the use of the novel compounds. The present invention furthermore relates to pharmaceutical compositions or combination preparations of the novel compounds as well as to said compositions or preparations for use as a medicine, more preferably for the prevention or treatment of diseases mediated by activity of YAP/TAZ-TEAD transcription, yet more in particular for the prevention or treatment of cancer or fibrosis. The present invention also relates to processes for the preparation of said compounds.

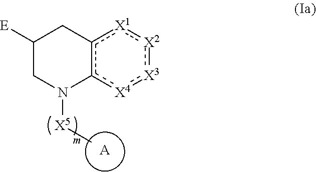

### FIELD

The present disclosure relates to novel compounds. The present disclosure also relates to said compounds for use as a medicine, more in particular for the prevention or treatment of diseases mediated by activity of YAP/TAZ-TEAD transcription, such as for the prevention or treatment of cancer or fibrosis. Methods for the prevention or treatment of said diseases comprising the use of the novel compounds are also disclosed herein.

The present disclosure furthermore relates to pharmaceutical compositions or combination preparations of the novel compounds as well as to said compositions or preparations for use as a medicine, more preferably for the prevention or treatment of diseases mediated by activity of YAP/TAZ-TEAD transcription, such as for the prevention or treatment of cancer or fibrosis.

Processes for the preparation of said compounds are also disclosed herein.

### BACKGROUND

Hippo signaling is critical to restrict organ size through inactivation of the YAP/TAZ-TEAD transcriptional complex. In several aggressive solid cancers, Hippo signaling is inactivated through loss-of-function mutations or deletions in the genes encoding the upstream regulators (e.g. NF2, MST1/2 or LATS1/2), unleashing constitutive YAP/TAZ-TEAD transcriptional activity leading to unbridled tumor growth and metastasis. Knock-out, knockdown or pharmacologic inactivation of YAP/TAZ-TEAD is sufficient to impair YAP/TAZ-dependent tumorigenesis. The YAP/TAZ-TEAD complex can be pharmacologically inactivated through targeted disruption of the YAP/TAZ-TEAD protein-protein interaction interface, or through an allosteric autopalmitoylation pocket in TEAD.

The main physiologic function of the Hippo pathway is to restrict tissue growth in adult tissue and modulate cell proliferation, differentiation and migration in developing organs. The core of the Hippo pathway consists of a kinase cascade, transcription coactivators and DNA-binding partners. In mammals, the Ste20-like kinases, MST1/2 (homologs of Drosophila Hippo) phosphorylate and activate Large Tumor Suppressor 1/2 (LATS1/2). NF2 is a scaffold for the core Hippo kinases, promoting LATS1/2 activation by tethering MST1/2 to LATS1/2 (Lallemand et al., 2003, Genes Dev 17, 1090-1100; Yin et al., 2013, Dev Cell 19, 27-38). The LATS kinases will in turn phosphorylate and inactivate two highly homologous transcriptional co-activators: Yes-associated Protein (YAP) and Transcriptional co-activator with PDZ-binding motif (TAZ) by cytoplasmic sequestration via 14-3-3 and by ubiquitin-mediated degradation induced by β-TRCP E3 ligase. When the Hippo pathway is inactive, YAP and TAZ translocate in the nucleus to bind to the TEAD transcription factor family to induce expression of a specific signature promoting matrix remodeling, cell proliferation, survival and migration. TEAD1-4 can also bind to VGLL4 in the nucleus and act as a transcriptional repressor. VGLL4 is not structurally related to YAP/TAZ, but competes with YAP/TAZ based on a partially overlapping binding site on TEAD (Johnson and Halder, 2014, Nat Rev Drug Discov 13, 63-79).

TEADs are evolutionarily conserved proteins required for cardiogenesis, myogenesis, and for the development of the neural crest, notochord, and trophoectoderm. In mammals, there are four genes encoding four homologous members of the TEAD family named TEAD1-4. Each TEAD gene has a distinct but not mutually exclusive expression pattern. All TEAD family members are controlled by YAP/TAZ.

In fruit flies, loss of function of Hippo or Warts kinases (MST1/2 or LATS1/2 in mammals), or overexpression of Yorkie (the Drosophila homolog of YAP and TAZ), results in a dramatic overgrowth of the cuticle, as a result of dysregulated cell proliferation and resistance to apoptosis, leading to increased organ size. In mice, YAP overexpression, loss of MST1/2 or LATS1/2 kinase activities, or loss of NF2 leads to TEAD target gene up-regulation and progenitor cell expansion, resulting in liver and cardiac overgrowth and ultimately cancer formation in the liver, the small intestine and in skin. In contrast, a serine to alanine mutation at position 94 in YAP, that is unable to bind to TEAD, is not oncogenic (Zhao et al., 2008, Genes Dev 22, 1962-1971). Likewise, a dominant-negative TEAD mutant that is unable to bind DNA, overcomes YAP-driven liver tumorigenesis. In addition, NF2 mutant liver carcinoma was greatly suppressed by heterozogous loss of Yap (Zhang et al., 2010, Dev Cell 19, 27-38). Finally, verteporfin, a small molecule that inhibits YAP-TEAD association significantly suppressed the oncogenic activity of YAP in these models (Liu-Chittenden et al., 2012, Genes Dev 26, 1300-1305).

Gene amplification of YAP1 (encoding for YAP) and WWTR1 (encoding for TAZ) as well as constitutive nuclear localization of YAP/TAZ have been reported in many human solid malignancies, including liver, lung, breast, skin, colon and ovarian cancer and YAP/TAZ promote the acquisition of several important cancer cell phenotypes, such as proliferation, resistance to apoptosis, invasion, and immune-suppression (e.g. by attracting myeloid derived suppressor cells (Wang et al., 2016, Cancer Discov 6, 80-95)). In addition, gene fusions with YAP1 have been identified in several cancer types including ependymomas, vascular cancers, cervical carcinomas and porocarcinomas, which results in constitutive activation of YAP-TEAD, and are oncogenic in mice (Szulzewsky et al., 2020, Genes Dev 34: 1-14). In addition, several germline or somatic mutations in components of the Hippo pathway associated with various cancer types have been discovered in targeted and whole-genome sequencing studies. The best studied example is the NF2 locus, mutated with a high frequency in neurofibromatosis. Loss of NF2 and LATS2 are also frequently observed in schwannomas. Another tumor type that is commonly (in about 70% of all cases) associated with constitutive YAP-TEAD activation through genetic inactivation of NF2, LATS1/2, MST1/2 or SAV1, is malignant mesothelioma (Bueno et al., 2016, Nat Genet 48, 407-416). Recent studies have shown that several mesothelioma cell lines with NF2 loss-of-function mutations exhibit a decrease in YAP phosphorylation and an increase in YAP-TEAD reporter activity. The YAP-TEAD transcription and viability of NF2 mutant mesothelioma cell lines (but not WT mesothelioma) are sensitive to YAP siRNA (an effect which can be rescued by overexpression of siRNA resistant YAP) and to treatment with verteporfin, a YAP antagonist (Zhang et al., 2017, J Cell Mol Med 21: 2663-2676).

Nuclear YAP has also emerged as a critical mediator of WNT dependent colorectal tumorigenesis. YAP-TEAD mediated transcription of genes involved in proliferation and stem cell renewal cooperate with WNT driven beta-catenin, and YAP is required for formation of adenomas following APC (adenomatous polyposis coli) inactivation (Azzolin et al., 2014 Cell 158, 157-170; Gregorieff et al., 2015 Nature 526, 715-718). Recently, TIAM1, was identified as a suppressor of aggressive, metastatic colorectal cancer (CRC) by antagonizing YAP-TEAD transcription, again highlighting the role of YAP-TEAD in CRC (Diamantopoulou et al., 2017 Cancer Cell 31, 621-634).

In summary, YAP/TAZ activation has been shown to drive tumorigenesis and YAP/TAZ is hyperactivated in many different types of cancer in humans (often through loss-of-function mutations in upstream negative regulators). Genetic deletion or pharmacologic inhibition of YAP/TAZ has been shown to suppress tumor development and progression in different types of cancer. Therefore, it is believed that deregulation of the Hippo tumor suppressor pathway is a major event in the development of a wide range of cancer types and malignancies. Hence, pharmacological targeting of the Hippo cascade through inhibition of YAP, TAZ, TEAD, and/or the YAP/TAZ-TEAD protein-protein interaction would be a valuable approach for the treatment of cancers that harbor functional alterations of this pathway.

YAP/TAZ-TEAD activation has also been shown to play an important role in other diseases than cancer, namely such as in fibrosis and certain congenital disorders. A hallmark of fibrosis is the excessive deposition of extracellular matrix (ECM), including cross-linked collagen fibres, which results in the stiffening of tissues and eventually in dysfunctioning of affected organs. ECM stiffening promotes the nuclear activity of YAP/TAZ in cancer-associated fibroblasts, and fibroblasts of the liver, kidney, lung and skin (Mannaerts et al., 2015, J. Hepatol. 63, 679-688; Piersma et al., 2015, Am. J. Pathol. 185, 3326-3337). Nuclear YAP/TAZ promotes fibrotic cellular phenotypes, such as myofibroblast differentiation and increased matrix remodeling. Several genes that encode key secreted factors implicated in fibrosis are direct YAP/TAZ-TEAD targets. These genes include well-characterized pro-fibrotic factors, such as connective tissue growth factor (CTGF), plasminogen activator inhibitor 1 (PAI-1) and the lysyl oxidase (LOX) family of collagen cross-linking enzymes. Several lines of evidence support YAP/TAZ as contributors to fibrotic disease in vivo. These include reports of elevated YAP/TAZ levels and transcriptional activity in fibroblasts as well as in alveolar and respiratory epithelium of patients with idiopathic pulmonary fibrosis (Gokey et al., 2018 JCI Insight 3: e98738). Increased nuclear YAP has also been observed in patients with primary sclerosing cholangitis and primary biliary cirrhosis, which are chronic fibrotic disorders of liver injury. Expression of YAP or TAZ in the duct cells of the liver drives fibrosis progression that parallels fibrosis in nonalcoholic fatty liver disease (Machado et al., 2015, J. Hepatol 63, 962-970). Collectively, these studies suggest that targeting aberrant YAP/TAZ activity in fibrotic diseases may hold promise for therapy.

Neurofibromatosis type 2 is characterized by nervous system tumors including schwannomas, meningiomas, and ependymomas. Neurofibromatosis type 2 is an inheritable disorder caused by the inactivation of NF2 (Striedinger et al., 2008, Neoplasia 10, 1204-1210). Loss of NF2 leads to constitutive activation of YAP/TAZ-TEAD. The Sturge-Weber syndrome is a congenital eurocutaneous disorder characterized by a port-wine stain affecting the skin in the distribution of the ophthalmic branch of the trigeminal nerve, abnormal capillary venous vessels in the leptomeninges of the brain and choroid, glaucoma, seizures, stroke, and intellectual disability. The Sturge-Weber syndrome and port-wine stains are caused by a somatic activating mutation in GNAQ which leads to activation of YAP/TAZ-TEAD transcription (Shirley et al., 2013, NEJM, 368, 1971-1979). Therefore, several congenital disorders, characterized by constitutive YAP/TAZ-TEAD activation could be treated with inhibitors of YAP/TAZ-TEAD.

A few publications describe inhibitors of the YAP-TEAD transcriptional activation. Inventiva highlighted YAP-TEAD protein-protein interaction inhibitors in WO2020/070181, WO2018/185266, and WO2017/064277. The General Hospital Corporation, Boston described autopalmitoylation inhibitors in WO2017/053706. Vivace Therapeutics, Inc. disclosed non-fused tricyclic (WO2018/204532), benzosulfonyl (WO2019/040380), benzocarbonyl (WO2019/113236), oxadiazole (WO2019/222431), and bicyclic (WO2020/097389) compounds that modulate the interaction between YAP/TAZ and TEAD. The Regents of the University of California and Vivace Therapeutics, Inc. described tricyclic compounds that inhibit the Hippo-YAP signaling pathway in WO2013/188138 and WO2017/058716, respectively. Kyowa Hakko Kirin Co., Ltd. revealed alpha,beta-unsaturated amide compounds that display anti-cancer activity in WO2018/235926 and US2019/0010136. Genentech, Inc. disclosed carboxamide and sulfonamide derivatives useful as inhibitors of the YAP-TEAD protein-protein interaction in WO2019/232216 and WO2020/051099. Dana-Farber Cancer Institute, Inc. highlighted inhibitors of TEAD transcription factors in WO2020/081572. The Trustees of Indiana University described small-molecules that bind within the hydrophobic palmitate-binding pocket of TEADs in WO2020/087063. Wenchao Lu, et al. published vinylsulfonamides as covalent TEAD autopalmitoylation inhibitors (2019, European Journal of Medicinal Chemistry, 184, p.111767). Korean Research Institute of Chemical Technology disclosed benzo[cd]indol-2(1H)-one derivatives that inhibit YAP-TEAD binding.

However, there is still a great need for novel, alternative or better therapeutics for the prevention or treatment of diseases mediated by the YAP/TAZ-TEAD activation, such as cancer and fibrosis among potentially other indications. Therapeutics with better potency, less side-effects, a higher activity, a lower toxicity or better pharmacokinetic or-dynamic properties or combinations thereof would be very welcome.

The present disclosure provides a class of novel compounds which can be used as inhibitors of the YAP/TAZ-TEAD activation mediated diseases.

### SUMMARY OF THE DISCLOSURE

The present disclosure is based on the finding that at least one of the above-mentioned problems can be solved by the below described class of compounds.

The present disclosure provides new compounds, especially a compound of formula (Ia), a stereo-isomeric form, a tautomer, a salt (in particular a pharmaceutically acceptable salt), solvate, polymorph and/or prodrug thereof,

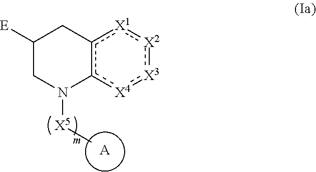

wherein:
E is selected from (5-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆alkyl)₂, —S(O)(NH)C₁₋₆alkyl, —S(O)(NC₁₋₆alkyl)C₁₋₆alkyl, —S(NH)(NH)C₁₋₆alkyl, —NH₂, —NHC₁₋₆alkyl, and —N(C₁₋₆alkyl)₂: and —(CR^{10a}R^{10b})ₙ—NR¹R²;
n is selected from 0; 1; and 2;
m is selected from 0; and 1;
each — represents an optional double bond, whereby maximally 3 —are a double bond at the same time;
R¹ is selected from C₁₋₆alkyl; C₃₋₉cycloalkyl; C₂₋₆alkenyl; C₅₋₉cycloalkenyl; C₂₋₆alkynyl; C₅₋₉ cycloalkynyl; C₁₋₆heteroalkyl; C₂₋₆heteroalkenyl; C₂₋₆heteroalkynyl; —C(O)H; —C(O)R³; —C(O)OR⁴; —C(O)NR⁵R⁶; —S(O)₂R^{3a}; —S(O)R^{4a}; —S(O)₂NR^{5a}R^{6a}; —S(O)(NR^{5a})R^{4a}; —S(NR^{5a})(NR^{6a})R^{3a}; and —P(O)R^{5b}R^{6b};
wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆alkyl)₂, —S(O)(NH)C₁₋₆alkyl, —S(O)(NC1-6alkyl)C₁₋₆ alkyl, —S(NH)(NH)C₁₋₆alkyl, —S(O)(NH)C₁₋₆alkyl, —S(O)(H)C1-6alkyl)C1-6alkyl, —S(O)₂OH, —S(NH)(NH)C1-6alkyl, —NH₂, —NHC₁₋₆alkyl, —N(C₁₋₆alkyl)₂;
R² is selected from hydrogen; C₁₋₆alkyl; C₃₋₉cycloalkyl; and C₁₋₆heteroalkyl;
R¹ and R² can be taken together to form a (4-; 5-; 6- or 7-membered) heterocycle which can be unsubstituted or substituted with one or more substituents selected from C₁₋₆alkyl, C₃₋₉ cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆alkyl)₂, —S(O)(NH)C1-6alkyl, —S(O)(NC₁₋₆alkyl)C₁₋₆alkyl, —S(NH)(NH)C₁₋₆alkyl, —NH₂, —NHC₁₋₆alkyl, —N(C₁₋₆alkyl)₂;
each R³ and R^{3a} is independently selected from hydroxyl; C₁₋₆alkyl; C₃₋₉cycloalkyl; C₂₋₆ alkenyl; C₅₋₉cycloalkenyl; C₂₋₆alkynyl; C₅₋₉cycloalkynyl; C₁₋₆heteroalkyl; C₂₋₆heteroalkenyl; and C₂₋₆ heteroalkynyl;
wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁴ and R^{4a} is independently selected from C₁₋₆alkyl; C₃₋₉cycloalkyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; and C₂₋₆ heteroalkynyl;
wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁵, R^{5a}, R^{5b}, R⁶, R^{6a} and R^{6b} is independently selected from hydrogen; C₁₋₆alkyl; C₃₋₉cycloalkyl; C₂₋₆alkenyl; C₅₋₉cycloalkenyl; C₂₋₆alkynyl; C₅₋₉cycloalkynyl; C₁₋₆heteroalkyl; C₂₋₆ heteroalkenyl; and C₂₋₆heteroalkynyl;
wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
and wherein each R⁵ and R⁶ or R^{5a} and R^{6a} can be taken together in order to form a (4-, 5-6-, or 7-membered) heterocycle which can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
cycle A is selected from aryl; heteroaryl; C₃₋₉cycloalkyl; and heterocycle;
wherein said aryl, heteroaryl, C₃₋₉cycloalkyl and heterocycle is substituted with one or more R⁷;
each R⁷ is independently selected from halogen; hydroxyl; sulfhydryl; ═O; ═S; —OZ1; —SZ1; —SCF3; —SF5; —CF₃; —OCF₃; —CHF₂; —OCHF₂; —NZ3Z4; —NZ3C(O)Z1; cyano; —C(O)Z2; —C(O)OZ¹; —C(O)NZ³Z⁴; C₁₋₆alkyl; C₃₋₉cycloakyl; C₂₋₆alkenyl; C₂₋₆alkynyl; C₁₋₆heteroalkyl; C₂₋₆ heteroalkenyl; C₂₋₆heteroalkynyl; aryl; heteroaryl; heterocycle; arylC₁₋₆alkyl; arylC₂₋₆alkenyl; arylalkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆ heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆ alkyl)₂;
X¹ is selected from CR⁸; N; and NR^{8a}; whereby X¹ can only be NR^{8a} when X² and/or X⁴ are C═O or C═S;
X² is selected from CR⁹; N; and NR^{9a}; whereby X² can only be NR^{9a} when X¹ and/or X³ are C═O or C—SH;
X³ is selected from CH; and N;
X⁴ is selected from CH; and N;
X⁵ is selected from —S(═O)₂—; —C(═O)—; and —CH₂—;
each R^{10a} is independently selected from hydrogen; and C₁₋₄alkyl;
each R^{10b} is independently selected from hydrogen; and C₁₋₄alkyl
wherein said alkyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
whereby maximally 2 of X¹, X², X³ and X⁴ can be a N (selected from N and NR^{8a} for X¹, from N and NR^{9a} for X², from N for X³, and from N for X⁴ respectively) at the same time;
provided that at least one of R⁸ and R⁹ is not hydrogen, each R⁸ and R⁹ are independently selected from hydrogen; halogen; hydroxyl; sulfhydryl; ═O; ═S; —OZ1a; —SZ1a; —SCF3; —SF5; —S(O)Z1a; —S(O)(NZ3a)Z1a; —S(NZ3a)(NZ3a)Z1a; —S(O)₂Z2a; —S(O)₂NZ3a Z4a; —CF₃; —OCF₃; —CHF₂; —OCHF₂; nitro; —NZ3a Z4a; —NZ3a S(O)₂Z1a; —NZ3a C(O)Z1a; —NZ3a C(O)NZ3a Z4a; cyano; —C(O)Z2; —C(O)OZ1a; —C(O)NZ3a Z4a; —C(O)H; —P(O)Z^{3a}Z4a; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆ heteroalkynyl; heteroarylC₁₋₆alkyl; heteroarylC₂₋₆alkenyl; heteroarylC₂₋₆alkynyl; heteroarylC₁₋₆ heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆ heteroalkenyl; and heterocycle-C₂₋₆heteroalkynyl;
wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆ heteroalkenyl, heteroarylC₂₋₆heteroalkynyl, heterocycle-C₁₋₆-akyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆ alkyl)₂;
each R^{8a} and R^{9a} are independently selected from hydrogen; hydroxyl; sulfhydryl; —OZ1a; —SZ1a; —SCF3; —SF5; —S(O)Z1a; —S(O)(NZ3a)Z1a; —S(NZ3a)(NZ3a)Z1a; —S(O)₂Z2a; —S(O)₂NZ3a Z4a; —CF₃; —OCF₃; —CHF₂; —OCHF₂; nitro; —NZ3a Z4a; —NZ3a S(O)2Z2a; —NZ3a C(O)Z1a; —NZ3a C(O)NZ3a Z4a; cyano; —C(O)Z2a; —C(O)OZ1a; —C(O)NZ3a Z4a; —C(O)H; —P(O)Z^{3a}Z4a; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆heteroalkenyl; heteroarylC₂₋₆ heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆ heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆ alkyl)₂;
each Z¹ and Z^{1a} is independently selected from C₁₋₆alkyl; C₃₋₉cycloakyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆heteroalkenyl; heteroarylC₂₋₆ heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆heteroalkyl, arylC₂₋₆heteroalkenyl, arylC₂₋₆ heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆ heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆ alkyl, and —N(C₁₋₆alkyl)₂;
each Z² and Z^{2a} is independently selected from hydroxyl; C₁₋₆alkyl; C₃₋₉cycloakyl; C₂₋₆ alkenyl; C₅₋₉cycloalkenyl; C₂₋₆alkynyl; C₅₋₉cycloalkynyl; C₁₋₆heteroalkyl; C₂₋₆heteroalkenyl; C₂₋₆ heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆ alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆ alkynyl; heterocycle-C₁₋₆heteroalkyl; heterocycle-C₂₋₆heteroalkenyl; and heterocycle-C₂₋₆ heteroalkynyl;
wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆heteroalkyl, arylC₂₋₆heteroalkenyl, arylC₂₋₆ heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆ heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆ alkyl, and —N(C₁₋₆alkyl)₂;
each Z³, Z^{3a}, Z⁴, and Z^{4a} is independently selected from hydrogen; C₁₋₆alkyl; C₃₋₉cycloakyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; C₂₋₆ heteroalkynyl; aryl; heteroaryl; heterocycle; arylC₁₋₆alkyl; arylC₂₋₆alkenyl; arylC₂₋₆alkynyl; arylC₁₋₆ heteroalkyl; arylC₂₋₆heteroalkenyl; arylC₂₋₆heteroalkynyl; heteroarylC₁₋₆alkyl; heteroarylC₂₋₆ alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆ alkynyl; heterocycle-C₁₋₆heteroalkyl; heterocycle-C₂₋₆heteroalkenyl; and heterocycle-C₂₋₆ heteroalkynyl;
wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆heteroalkyl, arylC₂₋₆heteroalkenyl, arylC₂₋₆ heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₂₋₆heteroalkyl, heterocycle-C₂₋₆ heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆ alkyl, and —N(C₁₋₆alkyl)₂;
and wherein each Z³ and Z⁴ or Z^{3a} and Z^{4a} can be taken together in order to form a (4-, 5-, 6-, or 7-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂.

The present disclosure also provides new compounds, especially a compound of formula (I), a stereo-isomeric form, a tautomer, a salt (in particular a pharmaceutically acceptable salt), solvate, polymorph and/or prodrug thereof,

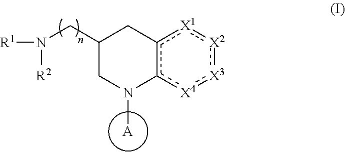

wherein:
n is selected from 0; 1; and 2;
each — represents an optional double bond, whereby maximally 3 —are a double bond at the same time;
R¹ is selected from alkyl; cycloalkyl; alkenyl; cycloalkenyl; alkynyl; cycloalkynyl; heteroalkyl; heteroalkenyl; heteroalkynyl; —C(O)H; —C(O)R³; —C(O)OR⁴; —C(O)NR⁵R⁶; —S(O)₂R^{3a}; —S(O)R^{4a}; —S(O)₂NR^{5a}R^{6a}; —S(O)(NR^{5a})R^{4a}; —S(NR^{5a})(NR^{6a})R^{3a}; and —P(O)R^{5b}R^{6b};
   wherein said alkyl, cycloalkyl, alkenyl, cycloalkenyl, alkynyl, cycloalkynyl, heteroalkyl, heteroalkenyl and heteroalkynyl can be unsubstituted or substituted with one or more substituents selected from alkyl, cycloalkyl, alkenyl, alkynyl, hydroxyl, ═O, halogen, —SH, ═S, trifluoromethyl, —O-alkyl, —OCF₃, cyano, nitro, —C(O)OH, —C(O)Oalkyl, —C(O)alkyl, —CONH₂, —CONHalkyl, —CON(alkyl)₂, —SO₂alkyl, —SO₂NH₂, —SO₂NHalkyl, —SO₂N(alkyl)₂, —S(O)(NH)alkyl, —S(O)(Nalkyl)alkyl, —S(NH)(NH)alkyl, —NH₂, —NHalkyl, —N(alkyl)₂;
R² is selected from hydrogen; alkyl; cycloalkyl; and heteroalkyl;
R¹ and R² can be taken together to form a (4-; 5-; 6- or 7-membered) heterocycle which can be unsubstituted or substituted with one or more substituents selected from alkyl, cycloalkyl, alkenyl, alkynyl, hydroxyl, ═O, halogen, —SH, ═S, trifluoromethyl, —O-alkyl, —OCF₃, cyano, nitro, —C(O)OH, —C(O)Oalkyl, —C(O)alkyl, —CONH₂, —CONHalkyl, —CON(alkyl)₂, —SO₂alkyl, —SO₂NH₂, —SO₂NHalkyl, —SO₂N(alkyl)₂, —S(O)(NH)alkyl, —S(O)(Nalkyl)alkyl, —S(NH)(NH)alkyl, —NH₂, —NHalkyl, —N(alkyl)₂;
each R³ and R^{3a} is independently selected from hydroxyl; alkyl; cycloalkyl; alkenyl; cycloalkenyl; alkynyl; cycloalkynyl; heteroalkyl; heteroalkenyl; and heteroalkynyl;
   wherein said alkyl, cycloalkyl, alkenyl, cycloalkenyl, alkynyl, cycloalkynyl, heteroalkyl, heteroalkenyl and heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁴ and R^{4a} is independently selected from alkyl; alkenyl; cycloalkenyl; alkynyl; cycloalkyl; cycloalkynyl; heteroalkyl; heteroalkenyl; and heteroalkynyl;
   wherein said alkyl, cycloalkyl, alkenyl, cycloalkenyl, alkynyl, cycloalkynyl, heteroalkyl, heteroalkenyl and heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁵, R^{5a}, R^{5b}, R⁶, R^{6a} and R^{6b} is independently selected from hydrogen; alkyl; cycloalkyl; alkenyl; cycloalkenyl; alkynyl; cycloalkynyl; heteroalkyl; heteroalkenyl; and heteroalkynyl;
   wherein said alkyl, cycloalkyl, alkenyl, cycloalkenyl, alkynyl, cycloalkynyl, heteroalkyl, heteroalkenyl and heteroalkynyl can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
   and wherein each R⁵ and R⁶ or R^{5a} and R^{6a} can be taken together in order to form a (4-, 5-, 6-, or 7-membered) heterocycle which can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂.
cycle A is selected from aryl; heteroaryl; cycloalkyl; and heterocycle;
   wherein said aryl, heteroaryl, cycloalkyl and heterocycle can be unsubstituted or substituted with one or more R⁷;
each R⁷ is independently selected from halogen; hydroxyl; sulfhydryl; ═O; ═S; —OZ¹; —SZ¹; —SCF3; —SF₅; —CF₃; —OCF₃; —CHF₂; —OCHF₂; —NZ³Z4; —NZ³C(O)Z¹; cyano; —C(O)Z²; —C(O)OZ¹; —C(O)NZ³Z4; alkyl; cycloakyl; alkenyl; alkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; and heterocycle-heteroalkynyl;
   wherein said alkyl, cycloakyl, alkenyl, alkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂; —OCHF₂, cyano, nitro, —C(O)OH, —NH₂, —NHalkyl, and —N(alkyl)₂;
X¹ is selected from CR⁸; N; and NR^{8a}; whereby X¹ can only be NR^{8a} when X² and/or X⁴ are C═O or C═S;
X² is selected from CR⁹; N; and NR^{9a}; whereby X² can only be NR^{9a} when X¹ and/or X³ are C═O or C—SH;
X³ is selected from CH; and N;
X⁴ is selected from CH; and N;
whereby maximally 2 of X¹, X², X³ and X⁴ can be a N (selected from N and NR^{8a} for X¹, from N and NR^{9a} for X², from N for X³, and from N for X⁴ respectively) at the same time;
each R⁸ and R⁹ are independently selected from hydrogen; halogen; hydroxyl; sulfhydryl; ═O; ═S; —OZ1a; —SZ1a; —SCF₃; —SF₅; —S(O)Z1a; —S(O)(NZ^{3a})Z1a; —S(NZ^{3a})(NZ^{3a})Z1a; —S(O)₂Z2a; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{1a}; —NZ^{3a}C(O)Z1a; —NZ^{3a}C(O)NZ^{3a}Z^{4a}; cyano; —C(O)Z2a; —C(O)OZ1a; —C(O)NZ^{3a}Z^{4a}; —C(O)H; —P(O)Z^{3a}Z4a; alkyl; cycloakyl; alkenyl; alkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; and heterocycle-heteroalkynyl;
   wherein said alkyl, cycloakyl, alkenyl, alkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂; —OCHF₂, cyano, nitro, —C(O)OH, —NH₂, —NHalkyl, and —N(alkyl)₂;
each R^{8a} and R^{9a} are independently selected from hydrogen; hydroxyl; sulfhydryl; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{2a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; alkyl; cycloakyl; alkenyl; alkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; and heterocycle-heteroalkynyl;
   wherein said alkyl, cycloakyl, alkenyl, alkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂; —OCHF₂, cyano, nitro, —C(O)OH, —NH₂, —NHalkyl, and —N(alkyl)₂;
each Z¹ and Z^{1a} is independently selected from alkyl; alkenyl; alkynyl; cycloalkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; or heterocycle-heteroalkynyl;
   wherein said alkyl, cycloalkyl, alkenyl, alkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each Z² and Z^{2a} is independently selected from hydroxyl; alkyl; cycloalkyl; alkenyl; alkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; or heterocycle-heteroalkynyl;
   wherein said alkyl, cycloalkyl, alkenyl, alkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each Z³, Z^{3a}, Z⁴, and Z^{4a} is independently selected from hydrogen; alkyl; cycloalkyl; alkenyl; alkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; or heterocycle-heteroalkynyl;
   wherein said alkyl, cycloalkyl, alkenyl, alkynyl, heteroalkyl, heteroalkenyl, heteroalkynyl, aryl, heteroaryl, heterocycle, cycloalkyl, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
   and wherein each Z³ and Z⁴ or Z^{3a} and Z^{4a} can be taken together in order to form a (4-, 5-, 6-, or 7-membered) heterocycle which can be unsubstituted or substituted with one or more substituents selected from alkyl, cycloalkyl, alkenylalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂.

The present disclosure provides new compounds which have been shown to possess inhibitory activity on the YAP/TAZ-TEAD transcription. The present disclosure furthermore demonstrates that these compounds efficiently inhibit the activity of YAP/TAZ-TEAD transcription. Therefore, these compounds constitute a useful class of new potent compounds that can be used in the treatment and/or prevention of Hippo mediated disorders in animals, mammals and humans, more specifically for the treatment and/or prevention of (i) cancer, more specifically lung cancer, breast cancer, head and neck cancer, oesophageal cancer, kidney cancer, bladder cancer, colon cancer, ovarian cancer, cervical cancer, endometrial cancer, liver cancer (including but not limited to cholangiocarcinoma), skin cancer, pancreatic cancer, gastric cancer, brain cancer and prostate cancer, mesotheliomas, and/or sarcomas (ii) fibrosis, and (iii) YAP/TAZ-TEAD activation related congenital disorders, among others.

In some aspects, the compounds described herein can be used in the treatment and/or prevention of Hippo mediated disorders in animals, mammals and humans, more specifically for the treatment and/or prevention of acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bronchogenic carcinoma, chondrosarcoma, chordoma, choriocarcinoma, chronic leukemia, chronic lymphocytic leukemia, chronic myelocytic (granulocytic) leukemia, chronic myelogenous leukemia, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, papillary adenocarcinomas, papillary carcinoma, pinealoma, polycythemia vera, rectal cancer, renal cell carcinoma, retinoblastoma, rhabdomyosarcoma, sarcoma, sebaceous gland carcinoma, seminoma, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

The present disclosure furthermore relates to the compounds of the invention for use as a medicine, to the use of such compounds as medicines and to their use for the manufacture of medicaments, more in particular for treating and/or preventing YAP/TAZ-TEAD activation mediated diseases, in particular (i) cancer, more specifically lung cancer, breast cancer, head and neck cancer, oesophageal cancer, kidney cancer, bladder cancer, colon cancer, ovarian cancer, cervical cancer, endometrial cancer, liver cancer (including but not limited to cholangiocarcinoma), skin cancer, pancreatic cancer, gastric cancer, brain cancer and prostate cancer, mesotheliomas, and/or sarcomas and (ii) fibrosis in animals or mammals, more in particular in humans. The invention also relates to methods for the preparation of all such compounds and to pharmaceutical compositions comprising them in an effective amount.

In some embodiments, the disclosure relates to the compounds of the invention for use as a medicine, to the use of such compounds as medicines and to their use for the manufacture of medicaments, more in particular for treating and/or preventing YAP/TAZ-TEAD activation mediated diseases more specifically for the treatment and/or prevention of acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bronchogenic carcinoma, chondrosarcoma, chordoma, choriocarcinoma, chronic leukemia, chronic lymphocytic leukemia, chronic myelocytic (granulocytic) leukemia, chronic myelogenous leukemia, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, papillary adenocarcinomas, papillary carcinoma, pinealoma, polycythemia vera, rectal cancer, renal cell carcinoma, retinoblastoma, rhabdomyosarcoma, sarcoma, sebaceous gland carcinoma, seminoma, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

The present disclosure also relates to a method of treatment or prevention of TEAD activation mediated disorders in humans by the administration of one or more such compounds, optionally in combination with one or more other medicines, to a patient in need thereof. The present disclosure also relates to methods of preparing the compounds disclosed herein comprising the steps for synthesis of the compounds described herein.

### FIGURES

FIG. 1: In vivo tumor growth inhibition with Cpd. No. 002 and Cpd. No. 086. Antitumor activity of Cpd. No. 002 and Cpd. No. 086 in the treatment of a mesothelioma NCl-H226 subcutaneous human lung cancer xenograft Model in Female Balb/c Nude Mice performed in accordance with example 86.

### DETAILED DESCRIPTION

The present invention will be further described and in some instances with respect to particular embodiments, but the invention is not limited thereto.

### Definitions

The term “YAP/TAZ-TEAD activation mediated diseases” refers to diseases in which hippo signaling is inactivated and whereby YAP/TAZ-TEAD activation is contributing, driving, sustaining, enabling or the like such disease. This might be through loss-of-function mutations or deletions in the genes encoding the upstream regulators of YAP/TAZ-TEAD (e.g. NF2, MST1/2,LATS1/2, FAT1 or SAV1), unleashing constitutive YAP-TEAD transcriptional activity leading to unbridled tumor growth and metastasis of some cancers. This might also be through YAP1 or WWTR1 (TAZ) gene amplifications, gene fusions or activating mutations, or YAP/TAZ overexpression or hyperactivity, among others. YAP/TAZ-TEAD activation mediated diseases therefore refers to cancer, but also includes fibrosis and certain congenital disorders. Cancers that are included in YAP/TAZ-TEAD mediated diseases are, without being limited thereto, lung cancer, breast cancer, head and neck cancer, oesophageal cancer, kidney cancer, bladder cancer, colon cancer, ovarian cancer, cervical cancer, endometrial cancer, liver cancer (including but not limited to cholangiocarcinoma), skin cancer, pancreatic cancer, gastric cancer, brain cancer and prostate cancer, mesotheliomas, and/or sarcomas. Also included are (i) squamous cell carcinomas of the lung, cervix, ovaries, head and neck, oesophagus, and/or skin, or (ii) cancers that originate from neuroectoderm-derived tissues, such as ependymomas, meningiomas, schwannomas, peripheral nerve-sheet tumors and/or neuroblastomas, or (iii) vascular cancers, such as epithelioid haemangioendotheliomas. Fibrotic diseases or fibrosis that is included in YAP/TAZ-TEAD mediated diseases are, without being limited thereto, liver fibrosis, lung fibrosis and heart fibrosis. Congenital disorders that are included in YAP/TAZ-TEAD mediated diseases are, without being limited thereto, Sturge-Weber syndrome and Neurofibromatosis type 2.

YAP/TAZ-TEAD mediated diseases also includes cancers that have developed resistance to prior treatments such has EGFR inhibitors, MEK inhibitors, AXL inhibitors, B-RAF inhibitors, RAS inhibitors and others.

The term “treat” or “treating” as used herein is intended to refer to administration of a compound or composition to a subject for the purpose of effecting a therapeutic benefit or prophylactic benefit through inhibition of the YAP/TAZ-TEAD transcription. Treating includes reversing, ameliorating, alleviating, inhibiting the progress of, lessening the severity of, or preventing a disease, disorder, or condition, or one or more symptoms of such disease, disorder or condition mediated through YAP/TAZ-TEAD transcription. By “therapeutic benefit” is meant eradication, amelioration, reversing, alleviating, inhibiting the progress of or lessening the severity of the underlying disorder being treated. Also, a therapeutic benefit is achieved with the eradication or amelioration of one or more of the physiological symptoms associated with the underlying disorder such that an improvement is observed in the patient, notwithstanding that the patient is afflicted with the underlying disorder in some embodiments. For prophylactic benefit, in some embodiments, the compositions are administered to a patient at risk of developing a particular disease, or to a patient reporting one or more of the physiological symptoms of a disease, even though a diagnosis of this disease has not been made.

The term “subject” as used herein, refers to an animal, for example a mammal, such as a human, a patient, who has been the object of treatment, observation or experiment or who is in need of such treatment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation or partial alleviation of the symptoms of the disease or disorder being treated.

The term “composition” as used herein is intended to encompass a product comprising the specified ingredients in the therapeutically effective amounts, as well as any product which results, directly or indirectly, from combinations of the specified ingredients in the specified amounts.

The term “antagonist” or “inhibitor” as used herein in reference to inhibitors of the YAP/TAZ-TEAD activation, refers to a compound capable of producing, depending on the circumstance, a functional antagonism of YAP/TAZ-TEAD activation.

It is to be noticed that the term “comprising”, used in the claims, should not be interpreted as being restricted to the means listed thereafter; it does not exclude other elements or steps.

Reference throughout this specification to “one embodiment” or “an embodiment” means that a particular feature, structure or characteristic described in connection with the embodiment is included in at least one embodiment of the disclosure. Furthermore, the particular features, structures or characteristics may be combined in any suitable manner, as would be apparent to one of ordinary skill in the art from this disclosure, in one or more embodiments. Where an indefinite or definite article is used when referring to a singular noun e.g. “a” or “an”, “the”, this includes a plural of that noun unless something else is specifically stated.

Similarly it should be appreciated that in the description of exemplary embodiments of the disclosure, various features of the disclosure are sometimes grouped together in a single embodiment, figure, or description thereof for the purpose of streamlining the disclosure and aiding in the understanding of one or more of the various inventive aspects.

In each of the following definitions, the number of carbon atoms represents the maximum number of carbon atoms generally optimally present in the substituent or linker; it is understood that where otherwise indicated in the present application, the number of carbon atoms represents the optimal maximum number of carbon atoms for that particular substituent or linker.

The term “leaving group” or “LG” as used herein means a chemical group which is susceptible to be displaced by a nucleophile or cleaved off or hydrolyzed in basic or acidic conditions. In a particular embodiment, a leaving group is selected from a halogen atom (e.g., C₁, Br, I) or a sulfonate (e.g., mesylate, tosylate, triflate).

The term “protecting group” refers to a moiety of a compound that masks or alters the properties of a functional group or the properties of the compound as a whole. The chemical substructure of a protecting group varies widely. One function of a protecting group is to serve as intermediates in the synthesis of the parental drug substance. Chemical protecting groups and strategies for protection/deprotection are well known in the art. See: “Protective Groups in Organic Chemistry”, Theodora W. Greene (John Wiley & Sons, Inc., New York, 1991. Protecting groups are often utilized to mask the reactivity of certain functional groups, to assist in the efficiency of desired chemical reactions, e.g. making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection, e.g. alcohols, be physiologically acceptable, although in general it is more desirable if the products are pharmacologically innocuous.

The term “alkyl” or “C₁₋₁₈alkyl” as used herein means C₁-C₁₈ normal, secondary, or tertiary, linear, branched or straight hydrocarbon with no site of unsaturation. Examples are methyl, ethyl, 1-propyl (n-propyl), 2-propyl (iPr), 1-butyl, 2-methyl-1-propyl(i-Bu), 2-butyl (s-Bu), 2-dimethyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, and n-icosyl. In particular embodiments, the term alkyl refers to C₁₋₁₂alkyl (C₁₋₁₂ hydrocarbons), yet more in particular to C₁₋₉alkyl (C₁₋₉ hydrocarbons), yet more in particular to C₁₋₆alkyl (C₁₋₆ hydrocarbons) as further defined herein above.

The term “haloalkyl” as a group or part of a group, refers to an alkyl group having the meaning as defined above wherein one, two, or three hydrogen atoms are each replaced with a halogen as defined herein. Non-limiting examples of such haloalkyl groups include chloromethyl, 1-bromoethyl, fluoromethyl, difluoromethyl, trifluoromethyl, 1,1,1-trifluoroethyl and the like.

The term “alkoxy” or “alkyloxy”, as a group or part of a group, refers to a group having the formula —OR^{b} wherein R^{b} is C₁₋₆alkyl as defined herein above. Non-limiting examples of suitable C₁₋₆alkoxy include methoxy, ethoxy, propoxy, isopropoxy, butoxy, isobutoxy, sec-butoxy, tert-butoxy, pentyloxy and hexyloxy.

The term “haloalkoxy”, as a group or part of a group, refers to a group of formula —O—R^{c}, wherein R^{c} is haloalkyl as defined herein. Non-limiting examples of suitable haloalkoxy include fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy, 2,2,2-trichloroethoxy, trichloromethoxy, 2-bromoethoxy, pentafluoroethyl, 3,3,3-trichloropropoxy, 4,4,4-trichlorobutoxy.

The term “cycloalkyl” or “C₃₋₁₈ cycloalkyl” as used herein and unless otherwise stated means a saturated hydrocarbon monovalent group having from 3 to 18 carbon atoms consisting of or comprising a C₃₋₁₀ monocyclic or C₇₋₁₈ polycyclic saturated hydrocarbon, such as for instance cyclopropyl, cyclobutyl, cyclopentyl, cyclopropylethylene, methylcyclopropylene, cyclohexyl, cycloheptyl, cyclooctyl, cyclooctylmethylene, norbornyl, fenchyl, trimethyltricycloheptyl, decalinyl, adamantyl and the like. In particular embodiments, the term cycloalkyl refers to C₃₋₁₂cycloalkyl (saturated cyclic C₃₋₁₂hydrocarbons), yet more in particular to C₃₋₉cycloalkyl (saturated cyclic C₃₋₉hydrocarbons), still more in particular to C₃₋₆cycloalkyl (saturated cyclic C₃₋₆hydrocarbons) as further defined herein above. For the avoidance of doubt, fused systems of a cycloalkyl ring with a heterocyclic ring are considered as heterocycle irrespective of the ring that is bound to the core structure. Fused systems of a cycloalkyl ring with an aryl ring are considered as aryl irrespective of the ring that is bound to the core structure. Fused systems of a cycloalkyl ring with a heteroaryl ring are considered as heteroaryl irrespective of the ring that is bound to the core structure.

The term “alkenyl” or “C₂₋₁₈alkenyl” as used herein is C₂-C₁₈ normal, secondary or tertiary, linear, branched or straight hydrocarbon with at least one site (usually 1 to 3, preferably 1) of unsaturation, namely a carbon-carbon, sp2 double bond. Examples include, but are not limited to: ethylene or vinyl (—CH═CH₂), allyl (—CH₂CH═CH₂), and 5-hexenyl (—CH₂CH₂CH₂CH₂CH═CH₂). The double bond may be in the cis or trans configuration. In particular embodiments, the term alkenyl refers to C₂₋₁₂alkenyl (C₂₋₁₂hydrocarbons), yet more in particular to C₂₋₆alkenyl (C₂₋₉ hydrocarbons), still more in particular to C₂₋₆ alkenyl (C₂₋₆hydrocarbons) as further defined herein above with at least one site (usually 1 to 3, preferably 1) of unsaturation, namely a carbon-carbon, sp2 double bond.

The term “alkenyloxy”, as a group or part of a group, refers to a group having the formula —OR^{d} wherein R^{d} is alkenyl as defined herein above.

The term “cycloalkenyl” as used herein refers to a non-aromatic hydrocarbon group having from 5 to 18 carbon atoms with at least one site (usually 1 to 3, preferably 1) of unsaturation, namely a carbon-carbon, sp2 double bond and consisting of or comprising a C₅₋₁₀ monocyclic or C₇₋₁₈ polycyclic hydrocarbon. Examples include, but are not limited to: cyclopentenyl (—C₅H₇), cyclopentenylpropylene, methylcyclohexenylene and cyclohexenyl (—C₆H₉). The double bond may be in the cis or trans configuration. In particular embodiments, the term cycloalkenyl refers to C₅₋₁₂ cycloalkenyl (cyclic C₅₋₁₂ hydrocarbons), yet more in particular to C₅₋₉ cycloalkenyl (cyclic C₅₋₉ hydrocarbons), still more in particular to C₅₋₆ cycloalkenyl (cyclic C₅₋₆ hydrocarbons) as further defined herein above with at least one site of unsaturation, namely a carbon-carbon, sp2 double bond. For the avoidance of doubt, fused systems of a cycloalkenyl ring with a heterocyclic ring are considered as heterocycle irrespective of the ring that is bound to the core structure. Fused systems of a cycloalkenyl ring with an aryl ring are considered as aryl irrespective of the ring that is bound to the core structure. Fused systems of a cycloalkenyl ring with a heteroaryl ring are considered as heteroaryl irrespective of the ring that is bound to the core structure.

The term “alkynyl” or “C₂₋₁₈alkynyl” as used herein refers to C₂-C₁₈ normal, secondary, tertiary, linear, branched or straight hydrocarbon with at least one site (usually 1 to 3, preferably 1) of unsaturation, namely a carbon-carbon, sp triple bond. Examples include, but are not limited to: ethynyl (—C═CH), 3-ethyl-cyclohept-1-ynylene, and 1-propynyl (propargyl, —CH₂C═CH). In particular embodiments, the term alkynyl refers to C₂₋₁₂ alkynyl (C₂₋₁₂ hydrocarbons), yet more in particular to C₂₋₉ alkynyl (C₂₋₉ hydrocarbons) yet more in particular to C₂₋₆alkynyl (C₂₋₆ hydrocarbons) as further defined herein above with at least one site (usually 1 to 3, preferably 1) of unsaturation, namely a carbon-carbon, sp triple bond.

The term “alkynyloxy”, as a group or part of a group, refers to a group having the formula —OR^{e} wherein R^{e} is alkynyl as defined herein above.

The term “cycloalkynyl” as used herein refers to a non-aromatic hydrocarbon group having from 5 to 18 carbon atoms with at least one site (usually 1 to 3, preferably 1) of unsaturation, namely a carbon-carbon, sp triple bond and consisting of or comprising a C₅₋₁₀ monocyclic or C₇₋₁₈ polycyclic hydrocarbon. Examples include, but are not limited to: cyclohept-1-yne, 3-ethyl-cyclohept-1-ynylene, 4-cyclohept-1-yn-methylene and ethylene-cyclohept-1-yne. In particular embodiments, the term cycloalkynyl refers to C₅₋₁₀ cycloalkynyl (cyclic C₅₋₁₀ hydrocarbons), yet more in particular to C₅₋₉ cycloalkynyl (cyclic C₅₋₉ hydrocarbons), still more in particular to C₅₋₆ cycloalkynyl (cyclic C₅₋₆ hydrocarbons) as further defined herein above with at least one site (usually 1 to 3, preferably 1) of unsaturation, namely a carbon-carbon, sp triple bond. For the avoidance of doubt, fused systems of a cycloalkynyl ring with a heterocyclic ring are considered as heterocycle irrespective of the ring that is bound to the core structure. Fused systems of a cycloalkynyl ring with an aryl ring are considered as aryl irrespective of the ring that is bound to the core structure. Fused systems of a cycloalkynyl ring with a heteroaryl ring are considered as heteroaryl irrespective of the ring that is bound to the core structure.

The term “alkylene” as used herein each refer to a saturated, branched or straight chain hydrocarbon group of 1-18 carbon atoms (more in particular C₁₋₁₂, C₁₋₉ or C₁₋₆ carbon atoms), and having two monovalent group centers derived by the removal of two hydrogen atoms from the same or two different carbon atoms of a parent alkane. Typical alkylene include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

The term “alkenylene” as used herein each refer to a branched or straight chain hydrocarbon of 2-18 carbon atoms (more in particular C₂₋₁₂, C₂₋₉ or C₂₋₆ carbon atoms) with at least one site (usually 1 to 3, preferably 1) of unsaturation, namely a carbon-carbon, sp2 double bond, and having two monovalent centers derived by the removal of two hydrogen atoms from the same or two different carbon atoms of a parent alkene.

The term “alkynylene” as used herein each refer to a branched or straight chain hydrocarbon of 2-18 carbon atoms (more in particular C₂₋₁₂, C₂₋₉ or C₂₋₆ carbon atoms) with at least one site (usually 1 to 3, preferably 1) of unsaturation, namely a carbon-carbon, sp triple bond, and having two monovalent centers derived by the removal of two hydrogen atoms from the same or two different carbon atoms of a parent alkyne.

The term “heteroalkyl” as used herein refers to an alkyl wherein one or more carbon atoms are replaced by one or more atoms selected from the group comprising oxygen, nitrogen or sulphur atom. The term heteroalkyl thus comprises —O—R^{b}, —NR^{c}—R^{b}, —R^{a}—O—R^{b}, and —S—R^{b}, wherein R^{a} is alkylene, R^{b} is alkyl, and R^{o} is hydrogen or alky as defined herein. In particular embodiments, the term refers to C₁₋₁₂heteroalkyl, C₁₋₆heteroalkyl or C₁₋₆heteroalkyl. In some embodiments heteroalkyl is selected from the group comprising alkyloxy, alkyl-oxy-alkyl, (mono or di)alkylamino, (mono or di-)alkyl-amino-alkyl, alkylthio, and alkyl-thio-alkyl.

The term “heteroalkenyl” as used herein refers to an acyclic alkenyl wherein one or more carbon atoms are replaced by one or more atoms selected from oxygen, nitrogen or sulphur atom. The term heteroalkenyl thus comprises —O—R^{d}, —NH—(R^{d}), —N(R^{d}))₂, —N(R^{b})(R^{d}), and —S—R^{d} wherein R^{b} is alkyl and R^{d} is alkenyl as defined herein. In particular embodiments, the term refers to C₂₋₁₂ heteroalkenyl, C₂₋₆heteroalkenyl or C₂₋₆heteroalkenyl. In some embodiments heteroalkenyl is selected from the group comprising alkenyloxy, alkenyl-oxy-alkenyl, (mono or di-)alkenylamino, (mono or di-)alkenyl-amino-alkenyl, alkenylthio, and alkenyl-thio-alkenyl, The term “heteroalkynyl” as used herein refers to an acyclic alkynyl wherein one or more carbon atoms are replaced by an oxygen, nitrogen or sulphur atom. The term heteroalkynyl thus comprises but is not limited to —O—R^{d}, —N(R^{d})₂, NHR^{d}, —N(R^{b})(R^{e}), —N(R^{d})(R^{e}), and —S—R^{d} wherein R^{b}is alkyl, R^{e} is alkynyl and R^{d} is alkenyl as defined herein. In particular embodiments, the term refers to C₂₁₂heteroalkynyl, C₂₋₉heteroalkynyl or C₂₋₆heteroalkynyl. In some embodiments the term heteroalkynyl is selected from the group comprising alkynyloxy, alkynyl-oxy-alkynyl, (mono or di-)alkynylamino, (mono or di-)alkynyl-amino-alkynyl, alkynylthio, alkynyl-thio-alkynyl,

The term “heteroalkylene” as used herein refers to an alkylene wherein one or more carbon atoms are replaced by one or more oxygen, nitrogen or sulphur atoms.

The term “heteroalkenylene” as used herein refers to an alkenylene wherein one or more carbon atoms are replaced by one or more oxygen, nitrogen or sulphur atoms.

The term “heteroalkynylene” as used herein refers to an alkynylene wherein one or more carbon atoms are replaced by one or more oxygen, nitrogen or sulphur atom.

The term “aryl” as used herein means an aromatic hydrocarbon of 6-20 carbon atoms derived by the removal of hydrogen from a carbon atom of a parent aromatic ring system. Typical aryl groups include, but are not limited to 1 ring, or 2 or 3 rings fused together, derived from benzene, naphthalene, anthracene, biphenyl, and the like. In particular embodiments, the term aryl refers to a 6-14 carbon atoms membered aromatic cycle, yet more in particular refers to a 6-10 carbon atoms membered aromatic cycle. Fused systems of an aryl ring with a cycloalkyl ring, or a cycloalkenyl ring, or a cycloalkynyl ring, are considered as aryl irrespective of the ring that is bound to the core structure. Fused systems of an aryl ring with a heterocycle are considered as heterocycle irrespective of the ring that is bound to the core structure. Thus, indoline, dihydrobenzofurane, dihydrobenzothiophene and the like are considered as heterocycle according to the invention. Fused systems of an aryl ring with a heteroaryl ring are considered as heteroaryl irrespective of the ring that is bound to the core structure.

The term “aryloxy”, as a group or part of a group, refers to a group having the formula —OR⁹ wherein R⁹ is aryl as defined herein above.

The term “arylalkyl” or “arylalkyl-” as used herein refers to an alkyl in which one of the hydrogen atoms bonded to a carbon atom, typically a terminal or sp3 carbon atom, is replaced with an aryl. Typical arylalkyl groups include, but are not limited to, benzyl, 2-phenylethan-1-yl, 2-phenylethen-1-yl, naphthylmethyl, 2-naphthylethyl, and the like. The arylalkyl group comprises 6 to 20 carbon atoms, e.g. the alkyl moiety of the arylalkyl group is 1 to 6 carbon atoms and the aryl moiety is 6 to 14 carbon atoms.

The term “arylalkyloxy”, as a group or part of a group, refers to a group having the formula —O—R^{a}—R^{g} wherein R^{g} is aryl, and R^{a} is alkylene as defined herein above.

The term “arylalkenyl” or “arylalkenyl-” as used herein refers to an alkenyl in which one of the hydrogen atoms bonded to a carbon atom, is replaced with an aryl. The arylalkenyl group comprises 6 to 20 carbon atoms, e.g. the alkenyl moiety of the arylalkenyl group is 1 to 6 carbon atoms and the aryl moiety is 6 to 14 carbon atoms.

The term “arylalkynyl” or “arylalkynyl-” as used herein refers to an alkynyl in which one of the hydrogen atoms bonded to a carbon atom, is replaced with an aryl. The arylalkynyl group comprises 6 to 20 carbon atoms, e.g. the alkynyl moiety of the arylalkynyl group is 1 to 6 carbon atoms and the aryl moiety is 6 to 14 carbon atoms.

The term “arylheteroalkyl” or “arylheteroalkyl-” as used herein refers to a heteroalkyl in which one of the hydrogen atoms bonded to a carbon atom, typically a terminal or sp3 carbon atom, is replaced with an aryl. The arylheteroalkyl group comprises 6 to 20 carbon atoms, e.g. the heteroalkyl moiety of the arylheteroalkyl group is 1 to 6 carbon atoms and the aryl moiety is 6 to 14 carbon atoms. In some embodiments arylheteroalkyl is selected from the group comprising aryl-O-alkyl, arylalkyl-O-alkyl, aryl-NH-alkyl, aryl-N(alkyl)₂, arylalkyl-NH-alkyl, arylalkyl-N-(alkyl)₂, aryl-S-alkyl, and arylalkyl-S-alkyl.

The term “arylheteroalkenyl” or “arylheteroalkenyl-” as used herein refers to a heteroalkenyl in which one of the hydrogen atoms bonded to a carbon atom, is replaced with an aryl. The arylheteroalkenyl group comprises 6 to 20 carbon atoms, e.g. the heteroalkenyl moiety of the arylheteroalkenyl group is 1 to 6 carbon atoms and the aryl moiety is 6 to 14 carbon atoms.

In some embodiments arylheteroalkenyl is selected from the group comprising aryl-O-alkenyl, arylalkenyl-O-alkenyl, aryl-NH-alkenyl, aryl-N(alkenyl)₂, arylalkenyl-NH-alkenyl, arylalkenyl-N-(alkenyl)₂, aryl-S-alkenyl, and arylalkenyl-S-alkenyl.

The term “arylheteroalkynyl” or “arylheteroalkynyl-” as used herein refers to a heteroalkynyl in which one of the hydrogen atoms bonded to a carbon atom, is replaced with an aryl. The arylheteroalkynyl group comprises 6 to 20 carbon atoms, e.g. the heteroalkynyl moiety of the arylheteroalkynyl group is 1 to 6 carbon atoms and the aryl moiety is 6 to 14 carbon atoms. In some embodiments arylheteroalkynyl is selected from the group comprising aryl-O-alkynyl, arylalkynyl-O-alkynyl, aryl-NH-alkynyl, aryl-N(alkynyl)₂, arylalkynyl-NH-alkynyl, arylalkynyl-N-(alkynyl)₂, aryl-S-alkynyl, and arylalkynyl-S-alkynyl.

The term “heterocycle” or “heterocyclyl” as used herein refer to non-aromatic, fully saturated or partially unsaturated ring system of 3 to 18 atoms including at least one N, O, S, or P (for example, 3 to 7 member monocyclic, 7 to 11 member bicyclic, or comprising a total of 3 to 10 ring atoms). Each ring of the heterocycle or heterocyclyl may have 1, 2, 3 or 4 heteroatoms selected from N, O and/or S, where the N and S heteroatoms may optionally be oxidized and the N heteroatoms may optionally be quaternized; and wherein at least one carbon atom of heterocyclyl can be oxidized to form at least one C═O. The heterocycle may be attached at any heteroatom or carbon atom of the ring or ring system, where valence allows. The rings of multi-ring heterocyclyls or heterocycles may be fused, bridged and/or joined through one or more spiro atoms. Fused systems of a heterocycle or heterocyclyl with an aryl ring are considered as heterocycle or heterocyclyl irrespective of the ring that is bound to the core structure. Fused systems of a heterocycle or heterocyclyl with a heteroaryl ring are considered as heteroaryl irrespective of the ring that is bound to the core structure.

Non limiting exemplary heterocycles or heterocyclic groups include piperidinyl, piperazinyl, homopiperazinyl, morpholinyl, tetrahydropyranyl, tetrahydrofuranyl, pyrrolidinyl, aziridinyl, oxiranyl, thiiranyl, azetidinyl, oxetanyl, thietanyl, 2-imidazolinyl, pyrazolidinyl imidazolidinyl, isoxazolinyl, oxazolidinyl, isoxazolidinyl, thiazolidinyl, isothiazolidinyl, succinimidyl, 3H-indolyl, indolinyl, isoindolinyl, chromanyl (also known as 3,4-dihydrobenzo[b]pyranyl), 2H-pyrrolyl, 1-pyrrolinyl, 2-pyrrolinyl, 3-pyrrolinyl, 4H-quinolizinyl, 2-oxopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, and morpholin-4-yl. The term “aziridinyl” as used herein includes aziridin-1-yl and aziridin-2-yl. The term “oxyranyl” as used herein includes oxyranyl-2-yl. The term “thiiranyl” as used herein includes thiiran-2-yl. The term “azetidinyl” as used herein includes azetidin-1-yl, azetidin-2-yl and azetidin-3-yl. The term “oxetanyl” as used herein includes oxetan-2-yl and oxetan-3-yl. The term “thietanyl” as used herein includes thietan-2-yl and thietan-3-yl. The term “pyrrolidinyl” as used herein includes pyrrolidin-1-yl, pyrrolidin-2-yl and pyrrolidin-3-yl. The term “tetrahydrofuranyl” as used herein includes tetrahydrofuran-2-yl and tetrahydrofuran-3-yl. The term “tetrahydrothiophenyl” as used herein includes tetrahydrothiophen-2-yl and tetrahydrothiophen-3-yl. The term “succinimidyl” as used herein includes succinimid-1-yl and succininmid-3-yl. The term “dihydropyrrolyl” as used herein includes 2,3-dihydropyrrol-1-yl, 2,3-dihydro-1H-pyrrol-2-yl, 2,3-dihydro-1H-pyrrol-3-yl, 2,5-dihydropyrrol-1-yl, 2,5-dihydro-1H-pyrrol-3-yl and 2,5-dihydropyrrol-5-yl. The term “2H-pyrrolyl” as used herein includes 2H-pyrrol-2-yl, 2H-pyrrol-3-yl, 2H-pyrrol-4-yl and 2H-pyrrol-5-yl. The term “3H-pyrrolyl” as used herein includes 3H-pyrrol-2-yl, 3H-pyrrol-3-yl, 3H-pyrrol-4-yl and 3H-pyrrol-5-yl. The term “dihydrofuranyl” as used herein includes 2,3-dihydrofuran-2-yl, 2,3-dihydrofuran-3-yl, 2,3-dihydrofuran-4-yl, 2,3-dihydrofuran-5-yl, 2,5-dihydrofuran-2-yl, 2,5-dihydrofuran-3-yl, 2,5-dihydrofuran-4-yl and 2,5-dihydrofuran-5-yl. The term “dihydrothiophenyl” as used herein includes 2,3-dihydrothiophen-2-yl, 2,3-dihydrothiophen-3-yl, 2,3-dihydrothiophen-4-yl, 2,3-dihydrothiophen-5-yl, 2,5-dihydrothiophen-2-yl, 2,5-dihydrothiophen-3-yl, 2,5-dihydrothiophen-4-yl and 2,5-dihydrothiophen-5-yl. The term “imidazolidinyl” as used herein includes imidazolidin-1-yl, imidazolidin-2-yl and imidazolidin-4-yl. The term “pyrazolidinyl” as used herein includes pyrazolidin-1-yl, pyrazolidin-3-yl and pyrazolidin-4-yl. The term “imidazolinyl” as used herein includes imidazolin-1-yl, imidazolin-2-yl, imidazolin-4-yl and imidazolin-5-yl. The term “pyrazolinyl” as used herein includes 1-pyrazolin-3-yl, 1-pyrazolin-4-yl, 2-pyrazolin-1-yl, 2-pyrazolin-3-yl, 2-pyrazolin-4-yl, 2-pyrazolin-5-yl, 3-pyrazolin-1-yl, 3-pyrazolin-2-yl, 3-pyrazolin-3-yl, 3-pyrazolin-4-yl and 3-pyrazolin-5-yl. The term “dioxolanyl” also known as “1,3-dioxolanyl” as used herein includes dioxolan-2-yl, dioxolan-4-yl and dioxolan-5-yl. The term “dioxolyl” also known as “1,3-dioxolyl” as used herein includes dioxol-2-yl, dioxol-4-yl and dioxol-5-yl. The term “oxazolidinyl” as used herein includes oxazolidin-2-yl, oxazolidin-3-yl, oxazolidin-4-yl and oxazolidin-5-yl. The term “isoxazolidinyl” as used herein includes isoxazolidin-2-yl, isoxazolidin-3-yl, isoxazolidin-4-yl and isoxazolidin-5-yl. The term “oxazolinyl” as used herein includes 2-oxazolinyl-2-yl, 2-oxazolinyl-4-yl, 2-oxazolinyl-5-yl, 3-oxazolinyl-2-yl, 3-oxazolinyl-4-yl, 3-oxazolinyl-5-yl, 4-oxazolinyl-2-yl, 4-oxazolinyl-3-yl, 4-oxazolinyl-4-yl and 4-oxazolinyl-5-yl. The term “isoxazolinyl” as used herein includes 2-isoxazolinyl-3-yl, 2-isoxazolinyl-4-yl, 2-isoxazolinyl-5-yl, 3-isoxazolinyl-3-yl, 3-isoxazolinyl-4-yl, 3-isoxazolinyl-5-yl, 4-isoxazolinyl-2-yl, 4-isoxazolinyl-3-yl, 4-isoxazolinyl-4-yl and 4-isoxazolinyl-5-yl. The term “thiazolidinyl” as used herein includes thiazolidin-2-yl, thiazolidin-3-yl, thiazolidin-4-yl and thiazolidin-5-yl. The term “isothiazolidinyl” as used herein includes isothiazolidin-2-yl, isothiazolidin-3-yl, isothiazolidin-4-yl and isothiazolidin-5-yl. The term “thiazolinyl” as used herein includes 2-thiazolinyl-2-yl, 2-thiazolinyl-4-yl, 2-thiazolinyl-5-yl, 3-thiazolinyl-2-yl, 3-thiazolinyl-4-yl, 3-thiazolinyl-5-yl, 4-thiazolinyl-2-yl, 4-thiazolinyl-3-yl, 4-thiazolinyl-4-yl and 4-thiazolinyl-5-yl. The term “isothiazolinyl” as used herein includes 2-isothiazolinyl-3-yl, 2-isothiazolinyl-4-yl, 2-isothiazolinyl-5-yl, 3-isothiazolinyl-3-yl, 3-isothiazolinyl-4-yl, 3-isothiazolinyl-5-yl, 4-isothiazolinyl-2-yl, 4-isothiazolinyl-3-yl, 4-isothiazolinyl-4-yl and 4-isothiazolinyl-5-yl. The term “piperidyl” also known as “piperidinyl” as used herein includes piperid-1-yl, piperid-2-yl, piperid-3-yl and piperid-4-yl. The term “dihydropyridinyl” as used herein includes 1,2-dihydropyridin-1-yl, 1,2-dihydropyridin-2-yl, 1,2-dihydropyridin-3-yl, 1,2-dihydropyridin-4-yl, 1,2-dihydropyridin-5-yl, 1,2-dihydropyridin-6-yl, 1,4-dihydropyridin-1-yl, 1,4-dihydropyridin-2-yl, 1,4-dihydropyridin-3-yl, 1,4-dihydropyridin-4-yl, 2,3-dihydropyridin-2-yl, 2,3-dihydropyridin-3-yl, 2,3-dihydropyridin-4-yl, 2,3-dihydropyridin-5-yl, 2,3-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 3,4-dihydropyridin-2-yl, 3,4-dihydropyridin-3-yl, 3,4-dihydropyridin-4-yl, 3,4-dihydropyridin-5-yl and 3,4-dihydropyridin-6-yl. The term “tetrahydropyridinyl” as used herein includes 1,2,3,4-tetrahydropyridin-1-yl, 1,2,3,4-tetrahydropyridin-2-yl, 1,2,3,4-tetrahydropyridin-3-yl, 1,2,3,4-tetrahydropyridin-4-yl, 1,2,3,4-tetrahydropyridin-5-yl, 1,2,3,4-tetrahydropyridin-6-yl, 1,2,3,6-tetrahydropyridin-1-yl, 1,2,3,6-tetrahydropyridin-2-yl, 1,2,3,6-tetrahydropyridin-3-yl, 1,2,3,6-tetrahydropyridin-4-yl, 1,2,3,6-tetrahydropyridin-5-yl, 1,2,3,6-tetrahydropyridin-6-yl, 2,3,4,5-tetrahydropyridin-2-yl, 2,3,4,5-tetrahydropyridin-3-yl, 2,3,4,5-tetrahydropyridin-3-yl, 2,3,4,5-tetrahydropyridin-4-yl, 2,3,4,5-tetrahydropyridin-5-yl and 2,3,4,5-tetrahydropyridin-6-yl. The term “tetrahydropyranyl” also known as “oxanyl” or “tetrahydro-2H-pyranyl”, as used herein includes tetrahydropyran-2-yl, tetrahydropyran-3-yl and tetrahydropyran-4-yl. The term “2H-pyranyl” as used herein includes 2H-pyran-2-yl, 2H-pyran-3-yl, 2H-pyran-4-yl, 2H-pyran-5-yl and 2H-pyran-6-yl. The term “4H-pyranyl” as used herein includes 4H-pyran-2-yl, 4H-pyran-3-yl and 4H-pyran-4-yl. The term “3,4-dihydro-2H-pyranyl” as used herein includes 3,4-dihydro-2H-pyran-2-yl, 3,4-dihydro-2H-pyran-3-yl, 3,4-dihydro-2H-pyran-4-yl, 3,4-dihydro-2H-pyran-5-yl and 3,4-dihydro-2H-pyran-6-yl. The term “3,6-dihydro-2H-pyranyl” as used herein includes 3,6-dihydro-2H-pyran-2-yl, 3,6-dihydro-2H-pyran-3-yl, 3,6-dihydro-2H-pyran-4-yl, 3,6-dihydro-2H-pyran-5-yl and 3,6-dihydro-2H-pyran-6-yl. The term “tetrahydrothiophenyl”, as used herein includes tetrahydrothiophen-2-yl, tetrahydrothiophenyl-3-yl and tetrahydrothiophenyl-4-yl. The term “2H-thiopyranyl” as used herein includes 2H-thiopyran-2-yl, 2H-thiopyran-3-yl, 2H-thiopyran-4-yl, 2H-thiopyran-5-yl and 2H-thiopyran-6-yl. The term “4H-thiopyranyl” as used herein includes 4H-thiopyran-2-yl, 4H-thiopyran-3-yl and 4H-thiopyran-4-yl. The term “3,4-dihydro-2H-thiopyranyl” as used herein includes 3,4-dihydro-2H-thiopyran-2-yl, 3,4-dihydro-2H-thiopyran-3-yl, 3,4-dihydro-2H-thiopyran-4-yl, 3,4-dihydro-2H-thiopyran-5-yl and 3,4-dihydro-2H-thiopyran-6-yl. The term “3,6-dihydro-2H-thiopyranyl” as used herein includes 3,6-dihydro-2H-thiopyran-2-yl, 3,6-dihydro-2H-thiopyran-3-yl, 3,6-dihydro-2H-thiopyran-4-yl, 3,6-dihydro-2H-thiopyran-5-yl and 3,6-dihydro-2H-thiopyran-6-yl. The term “piperazinyl” also known as “piperazidinyl” as used herein includes piperazin-1-yl and piperazin-2-yl. The term “morpholinyl” as used herein includes morpholin-2-yl, morpholin-3-yl and morpholin-4-yl. The term “thiomorpholinyl” as used herein includes thiomorpholin-2-yl, thiomorpholin-3-yl and thiomorpholin-4-yl. The term “dioxanyl” as used herein includes 1,2-dioxan-3-yl, 1,2-dioxan-4-yl, 1,3-dioxan-2-yl, 1,3-dioxan-4-yl, 1,3-dioxan-5-yl and 1,4-dioxan-2-yl. The term “dithianyl” as used herein includes 1,2-dithian-3-yl, 1,2-dithian-4-yl, 1,3-dithian-2-yl, 1,3-dithian-4-yl, 1,3-dithian-5-yl and 1,4-dithian-2-yl. The term “oxathianyl” as used herein includes oxathian-2-yl and oxathian-3-yl. The term “trioxanyl” as used herein includes 1,2,3-trioxan-4-yl, 1,2,3-trioxan-5-yl, 1,2,4-trioxan-3-yl, 1,2,4-trioxan-5-yl, 1,2,4-trioxan-6-yl and 1,3,4-trioxan-2-yl. The term “azepanyl” as used herein includes azepan-1-yl, azepan-2-yl, azepan-3-yl and azepan-4-yl. The term “homopiperazinyl” as used herein includes homopiperazin-1-yl, homopiperazin-2-yl, homopiperazin-3-yl and homopiperazin-4-yl. The term “indolinyl” as used herein includes indolin-1-yl, indolin-2-yl, indolin-3-yl, indolin-4-yl, indolin-5-yl, indolin-6-yl, and indolin-7-yl. The term “quinolizinyl” as used herein includes quinolizidin-1-yl, quinolizidin-2-yl, quinolizidin-3-yl and quinolizidin-4-yl. The term “isoindolinyl” as used herein includes isoindolin-1-yl, isoindolin-2-yl, isoindolin-3-yl, isoindolin-4-yl, isoindolin-5-yl, isoindolin-6-yl, and isoindolin-7-yl. The term “3H-indolyl” as used herein includes 3H-indol-2-yl, 3H-indol-3-yl, 3H-indol-4-yl, 3H-indol-5-yl, 3H-indol-6-yl, and 3H-indol-7-yl. The term “quinolizinyl” as used herein includes quinolizidin-1-yl, quinolizidin-2-yl, quinolizidin-3-yl and quinolizidin-4-yl. The term “quinolizinyl” as used herein includes quinolizidin-1-yl, quinolizidin-2-yl, quinolizidin-3-yl and quinolizidin-4-yl. The term “tetrahydroquinolinyl” as used herein includes tetrahydroquinolin-1-yl, tetrahydroquinolin-2-yl, tetrahydroquinolin-3-yl, tetrahydroquinolin-4-yl, tetrahydroquinolin-5-yl, tetrahydroquinolin-6-yl, tetrahydroquinolin-7-yl and tetrahydroquinolin-8-yl. The term “tetrahydroisoquinolinyl” as used herein includes tetrahydroisoquinolin-1-yl, tetrahydroisoquinolin-2-yl, tetrahydroisoquinolin-3-yl, tetrahydroisoquinolin-4-yl, tetrahydroisoquinolin-5-yl, tetrahydroisoquinolin-6-yl, tetrahydroisoquinolin-7-yl and tetrahydroisoquinolin-8-yl. The term “chromanyl” as used herein includes chroman-2-yl, chroman-3-yl, chroman-4-yl, chroman-5-yl, chroman-6-yl, chroman-7-yl and chroman-8-yl. The term “1H-pyrrolizine” as used herein includes 1H-pyrrolizin-1-yl, 1H-pyrrolizin-2-yl, 1H-pyrrolizin-3-yl, 1H-pyrrolizin-5-yl, 1H-pyrrolizin-6-yl and 1H-pyrrolizin-7-yl. The term “3H-pyrrolizine” as used herein includes 3H-pyrrolizin-1-yl, 3H-pyrrolizin-2-yl, 3H-pyrrolizin-3-yl, 3H-pyrrolizin-5-yl, 3H-pyrrolizin-6-yl and 3H-pyrrolizin-7-yl.

The term “heteroaryl” refers to an aromatic ring system of 5 to 18 atoms including at least one N, O, S, or P, containing 1 or 2 rings which can be fused together or linked covalently, each ring typically containing 5 to 6 atoms; at least one of said rings is aromatic, where the N and S heteroatoms may optionally be oxidized and the N heteroatoms may optionally be quaternized, and wherein at least one carbon atom of said heteroaryl can be oxidized to form at least one C═O. Fused systems of a heteroaryl ring with a cycloalkyl ring, or a cycloalkenyl ring, or a cycloalkynyl ring, are considered as heteroaryl irrespective of the ring that is bound to the core structure. Fused systems of a heteroaryl ring with a heterocycle are considered as heteroaryl irrespective of the ring that is bound to the core structure. Fused systems of a hetero aryl ring with an aryl ring are considered as heteroaryl irrespective of the ring that is bound to the core structure. Non-limiting examples of such heteroaryl, include: triazol-2-yl, pyridinyl, 1H-pyrazol-5-yl, pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, benzo[d]oxazol-2(3H)-one, 2,3-dihydro-benzofuranyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl; preferably said heteroaryl group is selected from the group comprising pyridyl, pyrazinyl, pyrimidinyl, pyrazolyl, pyrrolyl, isoxazolyl, thiophenyl, imidazolyl, indolyl, benzimidazolyl, s-triazinyl, oxazolyl, isothiazolyl, furyl, thienyl, triazolyl and thiazolyl; more preferably, said heteroaryl group is selected from the group comprising pyridyl, pyrazinyl, pyrimidinyl, indolyl and benzimidazolyl.

The term “pyrrolyl” (also called azolyl) as used herein includes pyrrol-1-yl, pyrrol-2-yl and pyrrol-3-yl. The term “furanyl” (also called “furyl”) as used herein includes furan-2-yl and furan-3-yl (also called furan-2-yl and furan-3-yl). The term “thiophenyl” (also called “thienyl”) as used herein includes thiophen-2-yl and thiophen-3-yl (also called thien-2-yl and thien-3-yl). The term “pyrazolyl” (also called 1H-pyrazolyl and 1,2-diazolyl) as used herein includes pyrazol-1-yl, pyrazol-3-yl or 1H-pyrazol-5-yl, pyrazol-4-yl and pyrazol-5-yl. The term “imidazolyl” as used herein includes imidazol-1-yl, imidazol-2-yl, imidazol-4-yl and imidazol-5-yl. The term “oxazolyl” (also called 1,3-oxazolyl) as used herein includes oxazol-2-yl, oxazol-4-yl and oxazol-5-yl. The term “isoxazolyl” (also called 1,2-oxazolyl), as used herein includes isoxazol-3-yl, isoxazol-4-yl, and isoxazol-5-yl. The term “thiazolyl” (also called 1,3-thiazolyl), as used herein includes thiazol-2-yl, thiazol-4-yl and thiazol-5-yl (also called 2-thiazolyl, 4-thiazolyl and 5-thiazolyl). The term “isothiazolyl” (also called 1,2-thiazolyl) as used herein includes isothiazol-3-yl, isothiazol-4-yl, and isothiazol-5-yl. The term “triazolyl” as used herein includes triazol-2-yl, 1H-triazolyl and 4H-1,2,4-triazolyl, “1H-triazolyl” includes 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl and 1H-1,2,4-triazol-5-yl. “4H-1,2,4-triazolyl” includes 4H-1,2,4-triazol-4-yl, and 4H-1,2,4-triazol-3-yl. The term “oxadiazolyl” as used herein includes 1,2,3-oxadiazol-4-yl, 1,2,3-oxadiazol-5-yl, 1,2,4-oxadiazol-3-yl, 1,2,4-oxadiazol-5-yl, 1,2,5-oxadiazol-3-yl and 1,3,4-oxadiazol-2-yl. The term “thiadiazolyl” as used herein includes 1,2,3-thiadiazol-4-yl, 1,2,3-thiadiazol-5-yl, 1,2,4-thiadiazol-3-yl, 1,2,4-thiadiazol-5-yl, 1,2,5-thiadiazol-3-yl (also called furazan-3-yl) and 1,3,4-thiadiazol-2-yl. The term “tetrazolyl” as used herein includes 1H-tetrazol-1-yl, 1H-tetrazol-5-yl, 2H-tetrazol-2-yl, and 2H-tetrazol-5-yl. The term “oxatriazolyl” as used herein includes 1,2,3,4-oxatriazol-5-yl and 1,2,3,5-oxatriazol-4-yl. The term “thiatriazolyl” as used herein includes 1,2,3,4-thiatriazol-5-yl and 1,2,3,5-thiatriazol-4-yl. The term “pyridinyl” (also called “pyridyl”) as used herein includes pyridin-2-yl, pyridin-3-yl and pyridin-4-yl (also called 2-pyridyl, 3-pyridyl and 4-pyridyl). The term “pyrimidyl” as used herein includes pyrimid-2-yl, pyrimid-4-yl, pyrimid-5-yl and pyrimid-6-yl. The term “pyrazinyl” as used herein includes pyrazin-2-yl and pyrazin-3-yl. The term “pyridazinyl as used herein includes pyridazin-3-yl and pyridazin-4-yl. The term “oxazinyl” (also called “1,4-oxazinyl”) as used herein includes 1,4-oxazin-4-yl and 1,4-oxazin-5-yl. The term “dioxinyl” (also called “1,4-dioxinyl”) as used herein includes 1,4-dioxin-2-yl and 1,4-dioxin-3-yl. The term “thiazinyl” (also called “1,4-thiazinyl”) as used herein includes 1,4-thiazin-2-yl, 1,4-thiazin-3-yl, 1,4-thiazin-4-yl, 1,4-thiazin-5-yl and 1,4-thiazin-6-yl. The term “triazinyl” as used herein includes 1,3,5-triazin-2-yl, 1,2,4-triazin-3-yl, 1,2,4-triazin-5-yl, 1,2,4-triazin-6-yl, 1,2,3-triazin-4-yl and 1,2,3-triazin-5-yl. The term “imidazo[2,1-b][1,3]thiazolyl” as used herein includes imidazo[2,1-b][1,3]thiazoi-2-yl, imidazo[2,1-b][1,3]thiazol-3-yl, imidazo[2,1-b][1,3]thiazol-5-yl and imidazo[2,1-b][1,3]thiazol-6-yl. The term “thieno[3,2-b]furanyl” as used herein includes thieno[3,2-b]furan-2-yl, thieno[3,2-b]furan-3-yl, thieno[3,2-b]furan-4-yl, and thieno[3,2-b]furan-5-yl. The term “thieno[3,2-b]thiophenyl” as used herein includes thieno[3,2-b]thien-2-yl, thieno[3,2-b]thien-3-yl, thieno[3,2-b]thien-5-yl and thieno[3,2-b]thien-6-yl. The term “thieno[2,3-d][1,3]thiazolyl” as used herein includes thieno[2,3-d][1,3]thiazol-2-yl, thieno[2,3-d][1,3]thiazol-5-yl and thieno[2,3-d][1,3]thiazol-6-yl. The term “thieno[2,3-d]imidazolyl” as used herein includes thieno[2,3-d]imidazol-2-yl, thieno[2,3-d]imidazol-4-yl and thieno[2,3-d]imidazol-5-yl. The term “tetrazolo[1,5-a]pyridinyl” as used herein includes tetrazolo[1,5-a]pyridine-5-yl, tetrazolo[1,5-a]pyridine-6-yl, tetrazolo[1,5-a]pyridine-7-yl, and tetrazolo[1,5-a]pyridine-8-yl. The term “indolyl” as used herein includes indol-1-yl, indol-2-yl, indol-3-yl,-indol-4-yl, indol-5-yl, indol-6-yl and indol-7-yl. The term “indolizinyl” as used herein includes indolizin-1-yl, indolizin-2-yl, indolizin-3-yl, indolizin-5-yl, indolizin-6-yl, indolizin-7-yl, and indolizin-8-yl. The term “isoindolyl” as used herein includes isoindol-1-yl, isoindol-2-yl, isoindol-3-yl, isoindol-4-yl, isoindol-5-yl, isoindol-6-yl and isoindol-7-yl. The term “benzofuranyl” (also called benzo[b]furanyl) as used herein includes benzofuran-2-yl, benzofuran-3-yl, benzofuran-4-yl, benzofuran-5-yl, benzofuran-6-yl and benzofuran-7-yl. The term “isobenzofuranyl” (also called benzo[c]furanyl) as used herein includes isobenzofuran-1-yl, isobenzofuran-3-yl, isobenzofuran-4-yl, isobenzofuran-5-yl, isobenzofuran-6-yl and isobenzofuran-7-yl. The term “benzothiophenyl” (also called benzo[b]thienyl) as used herein includes 2-benzo[b]thiophenyl, 3-benzo[b]thiophenyl, 4-benzo[b]thiophenyl, 5-benzo[b]thiophenyl, 6-benzo[b]thiophenyl and-7-benzo[b]thiophenyl (also called benzothien-2-yl, benzothien-3-yl, benzothien-4-yl, benzothien-5-yl, benzothien-6-yl and benzothien-7-yl). The term “isobenzothiophenyl” (also called benzo[c]thienyl) as used herein includes isobenzothien-1-yl, isobenzothien-3-yl, isobenzothien-4-yl, isobenzothien-5-yl, isobenzothien-6-yl and isobenzothien-7-yl. The term “indazolyl” (also called 1H-indazolyl or 2-azaindolyl) as used herein includes 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, and 2H-indazol-7-yl. The term “benzimidazolyl” as used herein includes benzimidazol-1-yl, benzimidazol-2-yl, benzimidazol-4-yl, benzimidazol-5-yl, benzimidazol-6-yl and benzimidazol-7-yl. The term “1,3-benzoxazolyl” as used herein includes 1,3-benzoxazol-2-yl, 1,3-benzoxazol-4-yl, 1,3-benzoxazol-5-yl, 1,3-benzoxazol-6-yl and 1,3-benzoxazol-7-yl. The term “1,2-benzisoxazolyl” as used herein includes 1,2-benzisoxazol-3-yl, 1,2-benzisoxazol-4-yl, 1,2-benzisoxazol-5-yl, 1,2-benzisoxazol-6-yl and 1,2-benzisoxazol-7-yl. The term “2,1-benzisoxazolyl” as used herein includes 2,1-benzisoxazol-3-yl, 2,1-benzisoxazol-4-yl, 2,1-benzisoxazol-5-yl, 2,1-benzisoxazol-6-yl and 2,1-benzisoxazol-7-yl. The term “1,3-benzothiazolyl” as used herein includes 1,3-benzothiazol-2-yl, 1,3-benzothiazol-4-yl, 1,3-benzothiazol-5-yl, 1,3-benzothiazol-6-yl and 1,3-benzothiazol-7-yl. The term “1,2-benzoisothiazolyl” as used herein includes 1,2-benzisothiazol-3-yl, 1,2-benzisothiazol-4-yl, 1,2-benzisothiazol-5-yl, 1,2-benzisothiazol-6-yl and 1,2-benzisothiazol-7-yl. The term “2,1-benzoisothiazolyl” as used herein includes 2,1-benzisothiazol-3-yl, 2,1-benzisothiazol-4-yl, 2,1-benzisothiazol-5-yl, 2,1-benzisothiazol-6-yl and 2,1-benzisothiazol-7-yl. The term “benzotriazolyl” as used herein includes benzotriazol-1-yl, benzotriazol-4-yl, benzotriazol-5-yl, benzotriazol-6-yl and benzotriazol-7-yl. The term “1,2,3-benzoxadiazolyl” as used herein includes 1,2,3-benzoxadiazol-4-yl, 1,2,3-benzoxadiazol-5-yl, 1,2,3-benzoxadiazol-6-yl and 1,2,3-benzoxadiazol-7-yl. The term “2,1,3-benzoxadiazolyl” as used herein includes 2,1,3-benzoxadiazol-4-yl, 2,1,3-benzoxadiazol-5-yl, 2,1,3-benzoxadiazol-6-yl and 2,1,3-benzoxadiazol-7-yl. The term “1,2,3-benzothiadiazolyl” as used herein includes 1,2,3-benzothiadiazol-4-yl, 1,2,3-benzothiadiazol-5-yl, 1,2,3-benzothiadiazol-6-yl and 1,2,3-benzothiadiazol-7-yl. The term “2,1,3-benzothiadiazolyl” as used herein includes 2,1,3-benzothiadiazol-4-yl, 2,1,3-benzothiadiazol-5-yl, 2,1,3-benzothiadiazol-6-yl and 2,1,3-benzothiadiazol-7-yl. The term “thienopyridinyl” as used herein includes thieno[2,3-b]pyridinyl, thieno[2,3-c]pyridinyl, thieno[3,2-c]pyridinyl and thieno[3,2-b]pyridinyl. The term “purinyl” as used herein includes purin-2-yl, purin-6-yl, purin-7-yl and purin-8-yl. The term “imidazo[1,2-a]pyridinyl”, as used herein includes imidazo[1,2-a]pyridin-2-yl, imidazo[1,2-a]pyridin-3-yl, imidazo[1,2-a]pyridin-4-yl, imidazo[1,2-a]pyridin-5-yl, imidazo[1,2-a]pyridin-6-yl and imidazo[1,2-a]pyridin-7-yl.

The term “1,3-benzodioxolyl”, as used herein includes 1,3-benzodioxol-4-yl, 1,3-benzodioxol-5-yl, 1,3-benzodioxol-6-yl, and 1,3-benzodioxol-7-yl. The term “quinolinyl” as used herein includes quinolin-2-yl, quinolin-3-yl, quinolin-4-yl, quinolin-5-yl, quinolin-6-yl, quinolin-7-yl and quinolin-8-yl. The term “isoquinolinyl” as used herein includes isoquinolin-1-yl, isoquinolin-3-yl, isoquinolin-4-yl, isoquinolin-5-yl, isoquinolin-6-yl, isoquinolin-7-yl and isoquinolin-8-yl. The term “cinnolinyl” as used herein includes cinnolin-3-yl, cinnolin-4-yl, cinnolin-5-yl, cinnolin-6-yl, cinnolin-7-yl and cinnolin-8-yl. The term “quinazolinyl” as used herein includes quinazolin-2-yl, quinazolin-4-yl, quinazolin-5-yl, quinazolin-6-yl, quinazolin-7-yl and quinazolin-8-yl. The term “quinoxalinyl” as used herein includes quinoxalin-2-yl, quinoxalin-5-yl, and quinoxalin-6-yl.

Heteroaryl and heterocycle or heterocyclyl as used herein includes by way of example and not limitation these groups described in Paquette, Leo A. “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566.

The term “heterocyclyloxy” or “heterocycleoxy”, as a group or part of a group, refers to a group having the formula —O—Rⁱ wherein Rⁱ is heterocyclyl as defined herein above.

The term “heterocyclylalkyloxy” or “heterocycleoxy”, as a group or part of a group, refers to a group having the formula —O—R^{a}—Rⁱ wherein Rⁱ is heterocyclyl, and R^{a} is alkyl as defined herein above.

The term “heteroaryloxy”, as a group or part of a group, refers to a group having the formula —O—R^{k} wherein R^{k} is heteroaryl as defined herein above.

The term “heteroarylalkyloxy”, as a group or part of a group, refers to a group having the formula —O—R^{a}—Rⁱ wherein Rⁱ is heteroaryl, and R^{a} is alkyl as defined herein above.

The term “heterocyclyl-alkyl” or “heterocycle-alkyl” as a group or part of a group, refers to an alkyl in which one of the hydrogen atoms bonded to a carbon atom, typically a terminal or sp3 carbon atom, is replaced with a heterocyclyl. A non-limiting example of a heterocyclyl-alkyl or heterocycle-alkyl group is 2-piperidinyl-methylene. The heterocyclyl-alkyl or heterocycle-alkyl group can comprise 6 to 20 atoms, e.g. the alkyl moiety is 1 to 6 carbon atoms and the heterocyclyl moiety is 3 to 14 atoms.

The term “heterocyclyl-alkenyl” or “heterocycle-alkenyl” as a group or part of a group refers to an alkenyl in which one of the hydrogen atoms bonded to a carbon atom, is replaced with an heterocyclyl. The heterocyclyl-alkenyl or heterocycle-alkenyl group can comprise 6 to 20 atoms, e.g. the alkenyl moiety is 2 to 6 carbon atoms and the heterocyclyl moiety is 3 to 14 atoms.

The term “heterocyclyl-alkynyl” or “heterocycle-alkynyl” as a group or part of a group refers to an alkynyl in which one of the hydrogen atoms bonded to a carbon atom, is replaced with a heterocyclyl. The heterocyclyl-alkynyl or heterocycle-alkynyl group can comprise 6 to 20 atoms, e.g. the alkynyl moiety can comprise 2 to 6 carbon atoms and the heterocyclyl moiety can comprise 3 to 14 atoms.

The term “heterocyclyl-heteroalkyl” or “heterocycle-heteroalkyl” as a group or part of a group refers to a heteroalkyl in which one of the hydrogen atoms bonded to a carbon atom, typically a terminal or sp3 carbon atom, is replaced with a heterocyclyl. The heterocyclyl-heteroalkyl or heterocycle-heteroalkyl group can comprise 6 to 20 atoms, e.g. the heteroalkyl moiety can comprise 1 to 6 carbon atoms and the heterocyclyl moiety can comprise 3 to 14 atoms.

In some embodiments heterocyclyl-heteroalkyl or heterocycle-heteroalkyl is selected from the group comprising heterocyclyl-O-alkyl, heterocyclylalkyl-O-alkyl, heterocyclyl-NH-alkyl, heterocyclyl-N(alkyl)₂, heterocyclylalkyl-NH-alkyl, heterocyclylalkyl-N-(alkyl)₂, heterocyclyl-S-alkyl, and heterocyclylalkyl-S-alkyl.

The term “heterocyclyl-heteroalkenyl” or “heterocycle-heteroalkenyl” as a group or part of a group refers to a heteroalkenyl in which one of the hydrogen atoms bonded to a carbon atom, is replaced with a heterocyclyl. The heterocyclyl-heteroalkenyl or heterocycle-heteroalkenyl group can comprise 6 to 20 atoms, e.g. the heteroalkenyl moiety can comprise 2 to 6 carbon atoms and the heterocyclyl moiety can comprise 3 to 14 atoms. In some embodiments heterocyclyl-heteroalkenyl or heterocycle-heteroalkenyl is selected from the group comprising heterocyclyl-O-alkenyl, heterocyclylalkyl-O-alkenyl, heterocyclyl-NH-alkenyl, heterocyclyl-N(alkenyl)₂, heterocyclylalkyl-NH-alkenyl, heterocyclylalkyl-N-(alkenyl)₂, heterocyclyl-S-alkenyl, and heterocyclylalkenyl-S-alkenyl.

The term “heterocyclyl-heteroalkynyl” or “heterocycle-heteroalkynyl” as a group or part of a group refers to a heteroalkynyl in which one of the hydrogen atoms bonded to a carbon atom, is replaced with a heterocyclyl. The heterocyclyl-heteroalkynyl or heterocycle-heteroalkynyl group can comprise 6 to 20 atoms, e.g. the heteroalkynyl moiety can comprise 2 to 6 carbon atoms and the heterocyclyl moiety can comprise 3 to 14 atoms. In some embodiments heterocyclyl-heteroalkynyl is selected from the group comprising heterocyclyl-O-alkynyl, heterocyclylalkynyl-O-alkynyl, heterocyclyl-NH-alkynyl, heterocyclyl-N(alkynyl)₂, heterocyclylalkynyl-NH-alkynyl, heterocyclylalkynyl-N-(alkynyl)₂, heterocyclyl-S-alkynyl, and heterocyclylalkynyl-S-alkynyl.

The term “heteroaryl-alkyl” as a group or part of a group refers to an alkyl in which one of the hydrogen atoms bonded to a carbon atom, typically a terminal or sp3 carbon atom, is replaced with a heteroaryl. An example of a heteroaryl-alkyl group is 2-pyridyl-methylene. The heteroaryl-alkyl group can comprise 6 to 20 atoms, e.g. the alkyl moiety of the heteroaryl-alkyl group can comprise 1 to 6 carbon atoms and the heteroaryl moiety can comprise 5 to 14 atoms.

The term “heteroaryl-alkenyl” as a group or part of a group refers to an alkenyl in which one of the hydrogen atoms bonded to a carbon atom, is replaced with an heteroaryl. The heteroaryl-alkenyl group can comprise 6 to 20 atoms, e.g. the alkenyl moiety of the heteroaryl-alkenyl group can comprise 2 to 6 carbon atoms and the heteroaryl moiety can comprise 5 to 14 atoms.

The term “heteroaryl-alkynyl” as a group or part of a group as used herein refers to an alkynyl in which one of the hydrogen atoms bonded to a carbon atom, is replaced with a heteroaryl.

The heteroaryl-alkynyl group comprises 6 to 20 atoms, e.g. the alkynyl moiety of the heteroaryl-alkynyl group is 2 to 6 carbon atoms and the heteroaryl moiety is 5 to 14 atoms.

The term “heteroaryl-heteroalkyl” as a group or part of a group as used herein refers to a heteroalkyl in which one of the hydrogen atoms bonded to a carbon atom, typically a terminal or sp3 carbon atom, is replaced with a heteroaryl. The heteroaryl-heteroalkyl group comprises 7 to 20 atoms, e.g. the heteroalkyl moiety of the heteroaryl-heteroalkyl group is 2 to 6 carbon atoms and the heteroaryl moiety is 5 to 14 atoms. In some embodiments heteroaryl-heteroalkyl is selected from the group comprising heteroaryl-O-alkyl, heteroarylalkyl-O-alkyl, heteroaryl-NH-alkyl, heteroaryl-N(alkyl)₂, heteroarylalkyl-NH-alkyl, heteroarylalkyl-N-(alkyl)₂, heteroaryl-S-alkyl, and heteroarylalkyl-S-alkyl.

The term “heteroaryl-heteroalkenyl” as a group or part of a group as used herein refers to a heteroalkenyl in which one of the hydrogen atoms bonded to a carbon atom, is replaced with an heteroaryl. The heteroaryl-heteroalkenyl group comprises 8 to 20 atoms, e.g. the heteroalkenyl moiety of the heteroaryl-heteroalkenyl group is 3 to 6 carbon atoms and the heteroaryl moiety is 5 to 14 atoms. In some embodiments heteroaryl-heteroalkenyl is selected from the group comprising heteroaryl-O-alkenyl, heteroarylalkenyl-O-alkenyl, heteroaryl-NH-alkenyl, heteroaryl-N(alkenyl)₂, heteroarylalkenyl-NH-alkenyl, heteroarylalkenyl-N-(alkenyl)₂, heteroaryl-S-alkenyl, and heteroarylalkenyl-S-alkenyl.

The term “heteroaryl-heteroalkynyl” as a group or part of a group as used herein refers to a heteroalkynyl in which one of the hydrogen atoms bonded to a carbon atom, is replaced with a heteroaryl. The heteroaryl-heteroalkynyl group comprises 8 to 20 atoms, e.g. the heteroalkynyl moiety of the heteroaryl-heteroalkynyl group is 2 to 6 carbon atoms and the heteroaryl moiety is 5 to 14 atoms. In some embodiments heteroaryl-heteroalkynyl is selected from the group comprising heteroaryl-O-alkynyl, heteroarylalkynyl-O-alkynyl, heteroaryl-NH-alkynyl, heteroaryl-N(alkynyl)₂, heteroarylalkynyl-NH-alkynyl, heteroarylalkynyl-N-(alkynyl)₂, heteroaryl-S-alkynyl, and heteroarylalkynyl-S-alkynyl.

By way of example, carbon bonded heteroaryl or heterocyclic rings (or heterocycles) can be bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heteroaryls and heterocyclyls include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl. By way of example, nitrogen bonded heterocyclic rings are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or ß-carboline. Still more typically, nitrogen bonded heteroaryls or heterocyclyls include 1-aziridyl, 1-azetedyl, 1-pyrrolyl, 1-imidazolyl, 1-pyrazolyl, and 1-piperidinyl.

As used herein and unless otherwise stated, the terms “alkoxy”, “cyclo-alkoxy”, “aryloxy”, “arylalkyloxy”, “heteroaryloxy” “heterocyclyloxy”, “alkylthio”, “cycloalkylthio”, “arylthio”, “arylalkylthio”, “heteroarylthio” and “heterocyclylthio” refer to substituents wherein an alkyl group, respectively a cycloalkyl, aryl, arylalkyl heteroaryl, or heterocyclyl (each of them such as defined herein), are attached to an oxygen atom or a sulfur atom through a single bond, such as but not limited to methoxy, ethoxy, propoxy, butoxy, thioethyl, thiomethyl, phenyloxy, benzyloxy, mercaptobenzyl and the like. The same definitions will apply for alkenyl and alkynyl instead of alkyl.

The term “alkylthio”, as a group or part of a group, refers to a group having the formula —S—R^{b} wherein R^{b} is alkyl as defined herein above. Non-limiting examples of alkylthio groups include methylthio (—SCH₃), ethylthio (—SCH₂CH₃), n-propylthio, isopropylthio, n-butylthio, isobutylthio, sec-butylthio, tert-butylthio and the like.

The term “alkenylthio”, as a group or part of a group, refers to a group having the formula —S—R^{d} wherein R^{d} is alkenyl as defined herein above.

The term “alkynylthio”, as a group or part of a group, refers to a group having the formula —S—R^{e} wherein R^{e} is alkynyl as defined herein above.

The term “arylthio”, as a group or part of a group, refers to a group having the formula —S—R⁹ wherein R⁹ is aryl as defined herein above.

The term “arylalkylthio”, as a group or part of a group, refers to a group having the formula —S—R^{a}—R^{g} wherein R^{a} is alkylene and R⁹ is aryl as defined herein above.

The term “heterocyclylthio”, as a group or part of a group, refers to a group having the formula —S—Rⁱ wherein Rⁱ is heterocyclyl as defined herein above.

The term “heteroarylthio”, as a group or part of a group, refers to a group having the formula —S—R^{k} wherein R^{k} is heteroaryl as defined herein above.

The term “heterocyclylalkylthio”, as a group or part of a group, refers to a group having the formula —S—R^{a}—Rⁱ wherein R^{a} is alkylene and Rⁱ is heterocyclyl as defined herein above.

The term “heteroarylalkylthio”, as a group or part of a group, refers to a group having the formula —S—R^{a}—R^{k} wherein R^{a} is alkylene and R^{k} is heteroaryl as defined herein above.

The term “mono- or di-alkylamino”, as a group or part of a group, refers to a group of formula —N(R^{c})(R^{b}) wherein R^{o} is hydrogen, or alkyl, R^{b} is alkyl. Thus, alkylamino include mono-alkyl amino group (e.g. mono-alkylamino group such as methylamino and ethylamino), and di-alkylamino group (e.g. di-alkylamino group such as dimethylamino and diethylamino). Non-limiting examples of suitable mono- or di-alkylamino groups include n-propylamino, isopropylamino, n-butylamino, i-butylamino, sec-butylamino, t-butylamino, pentylamino, n-hexylamino, di-n-propylamino, di-i-propylamino, ethylmethylamino, methyl-n-propylamino, methyl-1-propylamino, n-butylmethylamino, i-butylmethylamino, t-butylmethylamino, ethyl-n-propylamino, ethyl-1-propylamino, n-butylethylamino, i-butylethylamino, t-butylethylamino, di-n-butylamino, di-i-butylamino, methylpentylamino, methylhexylamino, ethylpentylamino, ethylhexylamino, propylpentylamino, propylhexylamino, and the like.

The term “mono- or di-arylamino”, as a group or part of a group, refers to a group of formula —N(R^{q})(R^{r}) wherein R^{q} and R^{r} are each independently selected from hydrogen, aryl, or alkyl, wherein at least one of R^{q} or R^{r} is aryl.

The term “mono- or di-heteroarylamino”, as a group or part of a group, refers to a group of formula —N(R^{u})(R^{v}) wherein R^{u} and R^{v} are each independently selected from hydrogen, heteroaryl, or alkyl, wherein at least one of R^{u} or R^{v} is heteroaryl as defined herein.

The term “mono- or di-heterocyclylamino”, as a group or part of a group, refers to a group of formula —N(R^{w})(R^{x}) wherein R^{w} and R^{x} are each independently selected from hydrogen, heterocyclyl, or alkyl, wherein at least one of R^{w} or R^{x} is heterocyclyl as defined herein.

As used herein and unless otherwise stated, the term halogen means any atom selected from the group consisting of fluorine (F), chlorine (Cl), bromine (Br) and iodine (I).

The terminology regarding a chemical group “which optionally includes one or more heteroatoms, said heteroatoms being selected from the atoms consisting of O, S, and N” as used herein, refers to a group where one or more carbon atoms are replaced by an oxygen, nitrogen or sulphur atom and thus includes, depending on the group to which is referred, heteroalkyl, heteroalkenyl, heteroalkynyl, heteroalkyl, heteroalkenyl, heteroalkynyl, cycloheteroalkyl, cycloheteroalkenyl, cycloheteroalkynyl, heteroaryl, arylheteroalkyl, heteroarylalkyl, heteroarylheteroalkyl, arylheteroalkenyl, heteroarylalkenyl, heteroarylheteroalkenyl, heteroarylheteroalkenyl, arylheteroalkynyl, heteroarylalkynyl, heteroarylheteroalkynyl, among others. This term therefore comprises, depending on the group to which is referred, as an example alkoxy, alkenyloxy, alkynyloxy, alkyl-O-alkylene, alkenyl-O-alkylene, arylalkoxy, benzyloxy, heteroaryl-heteroalkyl, heterocyclyl-heteroalkyl, heteroaryl-alkoxy, heterocyclyl-alkoxy, among others. As an example, the terminology “alkyl which optionally includes one or more heteroatoms, said heteroatoms being selected from the atoms consisting of O, S, and N” therefore refers to heteroalkyl, meaning an alkyl which comprises one or more heteroatoms in the hydrocarbon chain, whereas the heteroatoms may be positioned at the beginning of the hydrocarbon chain, in the hydrocarbon chain or at the end of the hydrocarbon chain. Examples of heteroalkyl include methoxy, methylthio, ethoxy, propoxy, CH₃—O—CH₂—, CH₃—S—CH₂—, CH₃—CH₂—O—CH₂—, CH₃—NH—, (CH₃)₂—N—, (CH₃)₂—CH₂—NH—CH₂—CH₂—, among many other examples. As an example, the terminology “arylalkylene which optionally includes one or more heteroatoms in the alkylene chain, said heteroatoms being selected from the atoms consisting of O, S, and N” therefore refers to arylheteroalkylene, meaning an arylalkylene which comprises one or more heteroatoms in the hydrocarbon chain, whereas the heteroatoms may be positioned at the beginning of the hydrocarbon chain, in the hydrocarbon chain or at the end of the hydrocarbon chain. “Arylheteroalkylene” thus includes aryloxy, arylalkoxy, aryl-alkyl-NH— and the like and examples are phenyloxy, benzyloxy, aryl-CH₂—S—CH₂—, aryl-CH₂—O—CH₂—, aryl-NH—CH₂— among many other examples. The same counts for “heteroalkenylene”, “heteroalkynylene”, and other terms used herein when referred to “which optionally includes one or more heteroatoms, said heteroatoms being selected from the atoms consisting of O, S, and N”.

The terminology regarding a chemical group “wherein optionally two or more hydrogen atoms on a carbon atom or heteroatom of said group can be taken together to form a ═O or ═S” as used herein, refers to a group where two or more hydrogen atoms on a carbon atom or heteroatom of said group are taken together to form ═O or ═S. As an example, the terminology refers to “an alkyl wherein optionally two or more hydrogen atoms on a carbon atom or heteroatom of said alkyl can be taken together to form a ═O or ═S”, includes among other examples CH₃—C(O)—CH₂—, CH₃—C(O)—, CH₃—C(S)—CH₂—, CH₃—S(O)₂—CH₂— and (CH₃)₂—CH₂—C(O)—CH₂—CH₂—.

The combination for a group “which optionally includes one or more heteroatoms, said heteroatoms being selected from the atoms consisting of O, S, and N” and “wherein optionally two or more hydrogen atoms on a carbon atom or heteroatom of said group can be taken together to form a ═O or ═S” can combine the two aspects described herein above and includes, if the group referred to is alkyl, among other examples CH₃—C(O)O—, CH₃—C(O)O—CH₂—, CH₃—NH—C(O)—, CH₃—C(O)—NH— CH₃—NH—C(O)—CH₂—, CH₃—NH—C(S)—CH₂—, CH₃—NH—C(S)—NH—CH₂—, CH₃—NH—S(O)₂— and CH₃—NH—S(O)₂—NH—CH₂—.

The term “single bond” as used herein for a linking group i.e. in a way that a certain linking group is selected from a single bond, etc. in the formulas herein, refers to a molecule wherein the linking group is not present and therefore refers to compounds with a direct linkage via a single bond between the two moieties being linked by the linking group.

As used herein with respect to a substituting group, and unless otherwise stated, the terms “substituted” such as in “substituted alkyl”, “substituted alkenyl”, substituted alkynyl”, “substituted aryl”, “substituted heteroaryl”, “substituted heterocyclyl”, “substituted arylalkyl”, “substituted heteroaryl-alkyl”, “substituted heterocyclyl-alkyl” and the like refer to the chemical structures defined herein, and wherein the said alkyl, alkenyl, alkynyl, group and/or the said aryl, heteroaryl, or heterocyclyl may be optionally substituted with one or more substituents (preferable 1, 2, 3, 4, 5 or 6), meaning that one or more hydrogen atoms are each independently replaced with at least one substituent. Typical substituents include, but are not limited to and in a particular embodiment said substituents are being independently selected from the group consisting of halogen, amino, hydroxyl, sulfhydryl, alkyl, alkoxy, alkenyl, alkenyloxy, alkynyl, alkynyloxy, cycloalkyl, cycloalkenyl, cycloalkynyl, heteroalkyl, heteroalkenyl, heteroalkynyl, aryl, heteroaryl, heterocyclyl, arylalkyl, arylalkenyl, arylalkynyl, cycloalkyl-alkyl, cycloalkylalkenyl, cycloalkylalkynyl, heteroaryl-alkyl, heterocyclyl-alkyl, heteroaryl-alkenyl, heterocyclyl-alkenyl and heteroaryl-alkynyl, heterocyclyl-alkynyl, —X, —Z, —O—, —OZ, ═O, —SZ, —S—, ═S, —NZ₂, —N⁺Z₃, ═NZ, ═N—OZ, —CX₃ (e.g. trifluoromethyl), —CN, —OCN, —SCN, —N═C═O, —N═C═S, —NO, —NO₂, ═N₂, —N₃, —NZC(O)Z, —NZC(S)Z, —NZC(O)O—, —NZC(O)OZ, —NZC(S)OZ, —NZC(O)NZZ, NZC(NZ)Z, NZC(NZ)NZZ, —C(O)NZZ, —C(NZ)Z, —S(O)₂O—, —S(O)₂OZ, —S(O)₂Z, —OS(O)₂OZ, —OS(O)₂Z, —OS(O)₂O—, —S(O)₂NZZ, —S(O)(NZ)Z, —S(O)Z, —OP(O)(OZ)₂, —P(O)(OZ)₂, —P(O)(OZ)₂, —P(O)(OZ)(O⁻), —P(O)(OH)₂, —C(O)Z, —C(O)X, —C(S)Z, —C(O)OZ, —C(O)O—, —C(S)OZ, —C(O)SZ, —C(S)SZ, —C(O)NZZ, —C(S)NZZ, —C(NZ)NZZ, —OC(O)Z, —OC(S)Z, —OC(O)O—, —OC(O)OZ, —OC(S)OZ, wherein each X is independently a halogen selected from F, Cl, Br, or I; and each Z is independently —H, alkyl, alkenyl, alkynyl, heteroalkyl, heteroalkenyl, heteroalkynyl, aryl, heteroaryl, heterocyclyl, cycloalkyl, protecting group or prodrug moiety, while two Z bonded to a nitrogen atom can be taken together with the nitrogen atom to which they are bonded to form a heteroaryl, or heterocyclyl. Alkyl(ene), alkenyl(ene), and alkynyl(ene) groups may also be similarly substituted.

Any substituent designation that is found in more than one site in a compound of this invention shall be independently selected.

Substituents optionally are designated with or without bonds. Regardless of bond indications, if a substituent is polyvalent (based on its position in the structure referred to), then any and all possible orientations of the substituent are intended.

As used herein and unless otherwise stated, the term “solvate” includes any combination which may be formed by a derivative of this invention with a suitable inorganic solvent (e.g. hydrates) or organic solvent, such as but not limited to alcohols, ketones, esters, ethers, nitriles and the like.

The term “heteroatom(s)” as used herein means an atom selected from nitrogen, which can be quaternized; oxygen; and sulfur, including sulfoxide and sulfone.

The term “hydroxy” as used herein means —OH.

The term “carbonyl” as used herein means carbon atom bonded to oxygen with a double bond, i.e., C═O.

The term “amino” as used herein means the —NH₂ group.

The present disclosure provides novel compounds which have been shown to possess YAP/TAZ-TEAD transcription inhibitory activity. The present invention furthermore demonstrates that these compounds efficiently inhibit TEAD activation and thereby inhibit YAP/TAZ-TEAD transcription activation. Therefore, these compounds constitute a useful class of new potent compounds that can be used in the treatment and/or prevention of YAP/TAZ-TEAD activation mediated diseases in subjects, more specifically for the treatment and/or prevention of cancer and fibrosis, among other diseases.

The present disclosure furthermore relates to the compounds for use as medicines and to their use for the manufacture of medicaments for treating and/or preventing cancer or fibrosis. The present disclosure relates to the compounds for use as medicines for treating and/or preventing YAP/TAZ-TEAD activation mediated diseases such as cancer or fibrosis in animals, mammals, more in particular in humans. The disclosure also relates to methods for the preparation of all such compounds and to pharmaceutical compositions comprising them in an effective amount. The present disclosure also relates to a method of treatment or prevention of cancer or fibrosis in humans by the administration of one or more such compounds, optionally in combination with one or more other medicines, to a patient in need thereof. The present disclosure also relates to the compounds for veterinary use and to their use as medicines for the prevention or treatment of diseases in a non-human mammal, such as cancer and fibrosis in non-human mammals.

More particularly, the compounds of the disclosure are compounds of formula (Ia) and any subgroup thereof as described herein, a stereo-isomeric form, a tautomer, a salt (in particular a pharmaceutically acceptable salt), solvate, polymorph and/or prodrug thereof,

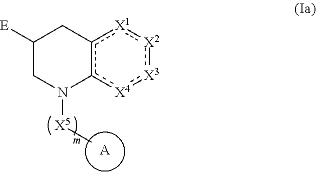

wherein:
E is selected from (5-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆ alkyl, —SO₂N(C₁₋₆alkyl)₂, —S(O)(NH)C₁₋₆alkyl, —S(O)(NC₁₋₆alkyl)C₁₋₆alkyl, —S(NH)(NH)C₁₋₆ alkyl, —NH₂, —NHC₁₋₆alkyl, and —N(C₁₋₆alkyl)₂; and —(CR^{10a}R^{10b})ₙ—NR¹R²;
n is selected from 0; 1; and 2;
m is selected from 0; and 1;
each — represents an optional double bond, whereby maximally 3 —are a double bond at the same time;
R¹ is selected from C₁₋₆alkyl; C₃₋₉cycloalkyl; C₂₋₆alkenyl; C₅₋₉cycloalkenyl; C₂₋₆alkynyl; C₅₋₉ cycloalkynyl; C₁₋₆heteroalkyl; C₂₋₆heteroalkenyl; C₂₋₆heteroalkynyl; —C(O)H; —C(O)R³; —C(O)OR⁴; —C(O)NR⁵R⁶; —S(O)₂R^{3a}; —S(O)R^{4a}; —S(O)₂NR^{5a}R^{6a}; —S(O)(NR^{5a})R^{4a}; —S(NR^{5a})(NR^{6a})R^{3a}; and —P(O)R^{5b}R^{6b};
   wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from C₁₋₆alkyl, C₃₋₉ cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆ alkyl)₂, —S(O)(NH)C₁₋₆alkyl, —S(O)(NC₁₋₆alkyl)C₁₋₆alkyl, —S(NH)(NH)C₁₋₆alkyl, —S(O)(NH)C₁₋₆alkyl, —S(O)(NC₁₋₆alkyl)C₁₋₆alkyl, —S(O)₂OH, —S(NH)(NH)C₁₋₆alkyl, —NH₂, —NHC₁₋₆alkyl, —N(C₁₋₆alkyl)₂;
R² is selected from hydrogen; C₁₋₆alkyl; C₃₋₉cycloalkyl; and C₁₋₆heteroalkyl;
R¹ and R² can be taken together to form a (4-; 5-; 6- or 7-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆ alkyl)₂, —S(O)(NH)C₁₋₆alkyl, —S(O)(NC₁₋₆alkyl)C₁₋₆alkyl, —S(NH)(NH)C₁₋₆alkyl, —NH₂, —NHC₁₋₆alkyl, —N(C₁₋₆alkyl)₂;
each R³ and R^{3a} is independently selected from hydroxyl; C₁₋₆alkyl; C₃₋₉cycloalkyl; C₂₋₆ alkenyl; C₅₋₉cycloalkenyl; C₂₋₆alkynyl; C₅₋₉cycloalkynyl; C₁₋₆heteroalkyl; C₂₋₆heteroalkenyl;
and C₂₋₆heteroalkynyl;
   wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁴ and R^{4a} is independently selected from C₁₋₆alkyl; C₃₋₉cycloalkyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; and C₂₋₆ heteroalkynyl;
   wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁵, R^{5a}, R^{5b}, R⁵, R^{5a} and R^{5b} is independently selected from hydrogen; C₁₋₆alkyl; C₃₋₉cycloalkyl; C₂₋₆alkenyl; C₅₋₉cycloalkenyl; C₂₋₆alkynyl; C₅₋₉cycloalkynyl; C₁₋₆heteroalkyl; C₂₋₆ heteroalkenyl; and C₂₋₆heteroalkynyl;
   wherein said C₁₋₆alkyl, C₃₋₉cycloalkyl, C₂₋₆alkenyl, C₅₋₉cycloalkenyl, C₂₋₆alkynyl, C₅₋₉cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
   and wherein each R⁵ and R⁶ or R^{5a} and R^{6a} can be taken together in order to form a (4-, 5-, 6-, or 7-membered) heterocycle which can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O— alkyl, —OCF₃, —CHF₂, —OCHF₂, cyano, nitro, —C(O)OH; NH₂; —NHalkyl, and —N(alkyl)₂;
cycle A is selected from aryl; heteroaryl; C₃₋₉cycloalkyl; and heterocycle;
   wherein said aryl, heteroaryl, C₃₋₉cycloalkyl and heterocycle is substituted with one or more R⁷;
each R⁷ is independently selected from halogen; hydroxyl; sulfhydryl; ═O; ═S; —OZ¹; —SZ¹; —SCF₃; —SF₅; —CF₃; —OCF₃; —CHF₂; —OCHF₂; —NZ³Z⁴; —NZ³C(O)Z¹; cyano; —C(O)Z²; —C(O)OZ¹; —C(O)NZ³Z⁴; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylalkynyl; arylC₁₋₆ heteroalkyl; arylC₂₋₆heteroalkenyl; arylC₂₋₆heteroalkynyl; heteroarylC₁₋₆alkyl; heteroarylC₂₋₆ alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆ alkynyl; heterocycle-C₁₋₆heteroalkyl; heterocycle-C₂₋₆heteroalkenyl; and heterocycle-C₂₋₆ heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆ heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆ alkyl, —OCF₃, —CHF₂; —OCHF₂, cyano, nitro, —C(O)OH, —NH₂′—NHC₁₋₆alkyl, and —N(C₁₋₆ alkyl)₂;
X¹ is selected from CR⁸; N; and NR^{8a}; whereby X¹ can only be NR^{8a} when X² and/or X⁴ are C═O or C═S;
X² is selected from CR⁹; N; and NR^{9a}; whereby X² can only be NR^{9a} when X¹ and/or X³ are C═O or C—SH;
X³ is selected from CH; and N;
X⁴ is selected from CH; and N;
X⁵ is selected from —S(═O)₂—; —C(═O)—; and —CH₂—;
each R^{10a} is independently selected from hydrogen; and C₁₋₄alkyl;
each R^{10b} is independently selected from hydrogen; and C₁₋₄alkyl
   wherein said alkyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
   whereby maximally 2 of X¹, X², X³ and X⁴ can be a N (selected from N and NR^{8a} for X¹, from N and NR^{9a} for X², from N for X³, and from N for X⁴ respectively) at the same time; provided that at least one of R⁸ and R⁹ is not hydrogen, each R⁸ and R⁹ are independently selected from hydrogen; halogen; hydroxyl; sulfhydryl; ═O; ═S; —OZ^{1a}; —SZ^{1a}; —SCF3; —SF₅; —S(O)Z^{1a}; —S(O)(NZ^{3a})Z^{1a}; —S(NZ^{3a})(NZ^{3a})Z^{1a}; —S(O)₂Z^{2a}—S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{1a}; —NZ^{3a}C(O)Z^{1a}; —NZ^{3a}C(O)NZ^{3a}Z⁴a cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆ heteroalkenyl; arylC₂₋₆heteroalkynyl; heteroarylC₁₋₆alkyl; heteroarylC₂₋₆alkenyl; heteroarylC₂₋₆ alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆ heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each R^{8a} and R^{9a} are independently selected from hydrogen; hydroxyl; sulfhydryl; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{2a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆-akyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each Z¹ and Z^{1a} is independently selected from C₁₋₆alkyl; C₃₋₉cycloakyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆heteroalkenyl; heteroarylC₂₋₆ heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₉alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆heteroalkyl, arylC₂₋₆ heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each Z² and Z^{2a} is independently selected from hydroxyl; C₁₋₆alkyl; C₃₋₉cycloakyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; C₂₋₆ heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆heteroalkyl, arylC₂₋₆ heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂;
each Z³, Z^{3a}, Z⁴, and Z^{4a} is independently selected from hydrogen; C₁₋₆alkyl; C₃₋₉cycloakyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆ alkenyl; arylC₂₋₆alkynyl; arylC₁₋₆heteroalkyl; arylC₂₋₆heteroalkenyl; arylC₂₋₆ heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₅₋₉cycloalkenyl, C₂₋₆alkynyl, C₅₋₉cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆ heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆ alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆ alkyl, and —N(C₁₋₆alkyl)₂;
and wherein each Z³ and Z⁴ or Z^{3a} and Z^{4a} can be taken together in order to form a (4-, 5-, 6-, or 7-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂.

The compounds of the disclosure also are compounds of formula (I) and any subgroup thereof as described herein, a stereo-isomeric form, a tautomer, a salt (in particular a pharmaceutically acceptable salt), solvate, polymorph and/or prodrug thereof,

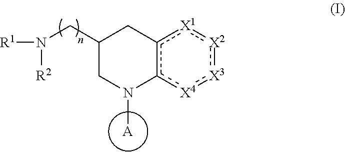

wherein:
n is selected from 0; 1; and 2;
each — represents an optional double bond, whereby maximally 3 —are a double bond at the same time;
R¹ is selected from alkyl; cycloalkyl; alkenyl; cycloalkenyl; alkynyl; cycloalkynyl; heteroalkyl; heteroalkenyl; heteroalkynyl; —C(O)H; —C(O)R³; —C(O)OR⁴; —C(O)NR⁵R⁶; —S(O)₂R^{3a}; —S(O)R^{4a}; —S(O)₂NR^{5a}R^{6a}; —S(O)(NR^{5a})R^{4a}; —S(NR^{5a})(NR^{6a})R^{3a}; and —P(O)R^{5b}R^{6b};
   wherein said alkyl, cycloalkyl, alkenyl, cycloalkenyl, alkynyl, cycloalkynyl, heteroalkyl, heteroalkenyl and heteroalkynyl can be unsubstituted or substituted with one or more substituents selected from alkyl, cycloalkyl, alkenyl, alkynyl, hydroxyl, ═O, halogen, —SH, ═S, trifluoromethyl, —O-alkyl, —OCF₃, cyano, nitro, —C(O)OH, —C(O)Oalkyl, —C(O)alkyl, —CONH₂, —CONHalkyl, —CON(alkyl)₂, —SO₂alkyl, —SO₂NH₂, —SO₂NHalkyl, —SO₂N(alkyl)₂, —S(O)(NH)alkyl, —S(O)(Nalkyl)alkyl, —S(NH)(NH)alkyl, —NH₂, —NHalkyl, —N(alkyl)₂;
R² is selected from hydrogen; alkyl; cycloalkyl; and heteroalkyl;
R¹ and R² can be taken together to form a (4-; 5-; 6- or 7-membered) heterocycle which can be unsubstituted or substituted with one or more substituents selected from alkyl, cycloalkyl, alkenyl, alkynyl, hydroxyl, ═O, halogen, —SH, ═S, trifluoromethyl, —O-alkyl, —OCF₃, cyano, nitro, —C(O)OH, —C(O)Oalkyl, —C(O)alkyl, —CONH₂, —CONHalkyl, —CON(alkyl)₂, —SO₂alkyl, —SO₂NH₂, —SO₂NHalkyl, —SO₂N(alkyl)₂, —S(O)(NH)alkyl, —S(O)(Nalkyl)alkyl, —S(NH)(NH)alkyl, —NH₂, —NHalkyl, —N(alkyl)₂;
each R³ and R^{3a} is independently selected from hydroxyl; alkyl; cycloalkyl; alkenyl; cycloalkenyl; alkynyl; cycloalkynyl; heteroalkyl; heteroalkenyl; and heteroalkynyl;
   wherein said alkyl, cycloalkyl, alkenyl, cycloalkenyl, alkynyl, cycloalkynyl, heteroalkyl, heteroalkenyl and heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁴ and R^{4a} is independently selected from alkyl; alkenyl; cycloalkenyl; alkynyl; cycloalkyl; cycloalkynyl; heteroalkyl; heteroalkenyl; and heteroalkynyl;
   wherein said alkyl, cycloalkyl, alkenyl, cycloalkenyl, alkynyl, cycloalkynyl, heteroalkyl, heteroalkenyl and heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁵, R^{5a}, R^{5b}, R⁶, R^{6a} and R^{6b} is independently selected from hydrogen; alkyl; cycloalkyl; alkenyl; cycloalkenyl; alkynyl; cycloalkynyl; heteroalkyl; heteroalkenyl; and heteroalkynyl;
   wherein said alkyl, cycloalkyl, alkenyl, cycloalkenyl, alkynyl, cycloalkynyl, heteroalkyl, heteroalkenyl and heteroalkynyl can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
   and wherein each R⁵ and R⁶ or R^{5a} and R^{6a} can be taken together in order to form a (4-, 5-, 6-, or 7-membered) heterocycle which can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂.
cycle A is selected from aryl; heteroaryl; cycloalkyl; and heterocycle;
   wherein said aryl, heteroaryl, cycloalkyl and heterocycle can be unsubstituted or substituted with one or more R⁷;
each R⁷ is independently selected from halogen; hydroxyl; sulfhydryl; ═O; ═S; —OZ¹; —SZ¹; —SCF₃; —SF₅; —CF₃; —OCF₃; —CHF₂; —OCHF₂; —NZ³Z⁴; —NZ³C(O)Z¹; cyano; —C(O)Z²; —C(O)OZ¹; —C(O)NZ³Z⁴; alkyl; cycloakyl; alkenyl; alkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; and heterocycle-heteroalkynyl;
   wherein said alkyl, cycloakyl, alkenyl, alkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂; —OCHF₂, cyano, nitro, —C(O)OH, —NH₂, —NHalkyl, and —N(alkyl)₂;
X¹ is selected from CR⁸; N; and NR^{8a}; whereby X¹ can only be NR^{8a} when X² and/or X⁴ are C═O or C═S;
X² is selected from CR⁹; N; and NR^{9a}; whereby X² can only be NR^{9a} when X¹ and/or X³ are C═O or C—SH;
X³ is selected from CH; and N;
X⁴ is selected from C^{H}; and N;
whereby maximally 2 of X¹, X², X³ and X⁴ can be a N (selected from N and NR^{8a} for X¹, from N and NR^{9a} for X², from N for X³, and from N for X⁴ respectively) at the same time;
each R⁸ and R⁹ are independently selected from hydrogen; halogen; hydroxyl; sulfhydryl; ═O; ═S; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{1a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; alkyl; cycloakyl; alkenyl; alkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; and heterocycle-heteroalkynyl;
   wherein said alkyl, cycloakyl, alkenyl, alkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂; —OCHF₂, cyano, nitro, —C(O)OH, —NH₂, —NHalkyl, and —N(alkyl)₂;
each R^{8a} and R^{9a} are independently selected from hydrogen; hydroxyl; sulfhydryl; —OZ^{1a}; —SZ^{1a}; —SCF3; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{2a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; alkyl; cycloakyl; alkenyl; alkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; and heterocycle-heteroalkynyl;
   wherein said alkyl, cycloakyl, alkenyl, alkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂; —OCHF₂, cyano, nitro, —C(O)OH, —NH₂, —NHalkyl, and —N(alkyl)₂;
each Z¹ and Z^{1a} is independently selected from alkyl; alkenyl; alkynyl; cycloalkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; or heterocycle-heteroalkynyl;
   wherein said alkyl, cycloalkyl, alkenyl, alkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each Z² and Z^{2a} is independently selected from hydroxyl; alkyl; cycloalkyl; alkenyl; alkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; or heterocycle-heteroalkynyl;
   wherein said alkyl, cycloalkyl, alkenyl, alkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each Z³, Z^{3a}, Z⁴, and Z^{4a} is independently selected from hydrogen; alkyl; cycloalkyl; alkenyl; alkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; or heterocycle-heteroalkynyl;
   wherein said alkyl, cycloalkyl, alkenyl, alkynyl, heteroalkyl, heteroalkenyl, heteroalkynyl, aryl, heteroaryl, heterocycle, cycloalkyl, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
   and wherein each Z³ and Z⁴ or Z^{3a} and Z^{4a} can be taken together in order to form a (4-, 5-, 6-, or 7-membered) heterocycle which can be unsubstituted or substituted with one or more substituents selected from alkyl, cycloalkyl, alkenylalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂. provided that the compounds are not selected from:
Acetamide, N-[1-(1-cyclopentyl-1H-tetrazol-5-yl)-1,2,3,4-tetrahydro-3quinolinyl]-;
Acetamide, N-[(1,2,3,4-tetrahydro-1-phenyl-3-quinolinyl)methyl]-;
Propanamide, N-(1,2,3,4-tetrahydro-1-phenyl-3-quinolinyl)-;
Cyclopropanecarboxamide, N-[1,2,3,4-tetrahydro-1-(3-methoxyphenyl)-3-quinolinyl]-;
Cyclohexaneacetamide, N-(6-cyano-1-cyclohexyl-1,2,3,4-tetrahydro-3-quinolinyl)-;
Carbamic acid, N-[1-(3,4-dichlorophenyl)-1,2,3,4-tetrahydro-3-quinolinyl]-, 1,1-dimethylethyl ester; Carbamic acid, N-[1-(3,4-dichlorophenyl)-1,2,3,4-tetrahydro-3-quinolinyl]—N-methyl-, 1,1-dimethylethyl ester; Carbamic acid, N-[(3S)-1-(3,4-dichlorophenyl)-1,2,3,4-tetrahydro-3-quinolinyl]-, 1,1-dimethylethyl ester; Carbamic acid, N-[(3R)-1-(3,4-dichlorophenyl)-1,2,3,4-tetrahydro-3-quinolinyl]-, 1,1-dimethylethyl ester; Carbamic acid, N-(1,2,3,4-tetrahydro-1-phenyl-3-quinolinyl)-, 1,1-dimethylethyl ester;
compounds wherein cycle A is N-[1-[1-(2,2-dimethylpropyl)-2,3-dihydro-3-methyl-2-oxo-1Himidazo[4,5-b]pyridin-5-yl] or N-[1-[1-[(2,2-difluoro-1-methylcyclopropyl)methyl]-2,3-dihydro-3-methyl-2-oxo-1H-imidazo[4,5-b]pyridin-5-yl]; and
compounds wherein R¹ is methyl or pentyl.

Preferred or particular statements (features) and embodiments of the compounds of this disclosure are set herein below. Each statement, aspect and embodiment of the disclosure so defined may be combined with any other statement, aspect and/or embodiment, unless clearly indicated to the contrary. In particular, any feature indicated as being preferred, particular or advantageous may be combined with any other features or statements indicated as being preferred, particular or advantageous. Hereto, the present disclosure is in particular captured by any one or any combination of one or more of the below numbered statements and embodiments, with any other statement, aspect and/or embodiment (which are not numbered).

1. A compound of Formula (Ia), or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof,

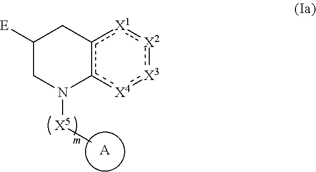

wherein:
E is selected from (5-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆ alkyl, —SO₂N(C₁₋₆alkyl)₂, —S(O)(NH)C₁₋₆alkyl, —S(O)(NC₁₋₆alkyl)C₁₋₆alkyl, —S(NH)(NH)C₁₋₆ alkyl, —NH₂, —NHC₁₋₆alkyl, and —N(C₁₋₆alkyl)₂; and —(CR^{10a}R^{10b})ₙ—NR¹R²;
n is selected from 0; 1; and 2;
m is selected from 0; and 1;
each — represents an optional double bond, whereby maximally 3 —are a double bond at the same time;
R¹ is selected from C₁₋₆alkyl; C₃₋₉cycloalkyl; C₂₋₆alkenyl; C₅₋₉cycloalkenyl; C₂₋₆alkynyl; C₅₋₉ cycloalkynyl; C₁₋₆heteroalkyl; C₂₋₆heteroalkenyl; C₂₋₆heteroalkynyl; —C(O)H; —C(O)R³; —C(O)OR⁴; —C(O)NR⁵R⁶; —S(O)₂R^{3a}; —S(O)R^{4a}; —S(O)₂NR^{5a}R^{6a}; —S(O)(NR^{5a})R^{4a}; —S(NR^{5a})(NR^{6a})R^{3a}; and —P(O)R^{5b}R^{6b};
   wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from C₁₋₆alkyl, C₃₋₉ cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆ alkyl)₂, —S(O)(NH)C₁₋₆alkyl, —S(O)(NC₁₋₆alkyl)C₁₋₆alkyl, —S(NH)(NH)C₁₋₆alkyl, —S(O)(NH)C₁₋₆alkyl, —S(O)(NC₁₋₆alkyl)C₁₋₆alkyl, —S(O)₂OH, —S(NH)(NH)C₁₋₆alkyl, —NH₂, —NHC₁₋₆alkyl, —N(C₁₋₆alkyl)₂;
R² is selected from hydrogen; C₁₋₆alkyl; C₃₋₉cycloalkyl; and C₁₋₆heteroalkyl;
R¹ and R² can be taken together to form a (4-; 5-; 6- or 7-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆ alkyl)₂, —S(O)(NH)C₁₋₆alkyl, —S(O)(NC₁₋₆alkyl)C₁₋₆alkyl, —S(NH)(NH)C₁₋₆alkyl, —NH₂, —NHC₁₋₆alkyl, —N(C₁₋₆alkyl)₂;
each R³ and R^{3a} is independently selected from hydroxyl; C₁₋₆alkyl; C₃₋₉cycloalkyl; C₂₋₆ alkenyl; C₅₋₉cycloalkenyl; C₂₋₆alkynyl; C₅₋₉cycloalkynyl; C₁₋₆heteroalkyl; C₂₋₆heteroalkenyl; and C₂₋₆heteroalkynyl;
   wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁴ and R^{4a} is independently selected from C₁₋₆alkyl; C₃₋₉cycloalkyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; and C₂₋₆ heteroalkynyl;
   wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁵, R^{5a}, R^{5b}, R⁵, R^{5a} and R^{5b} is independently selected from hydrogen; C₁₋₆alkyl; C₃₋₆ cycloalkyl; C₂₋₆alkenyl; C₅₋₉cycloalkenyl; C₂₋₆alkynyl; C₅₋₉cycloalkynyl; C₁₋₆heteroalkyl; C₂₋₆ heteroalkenyl; and C₂₋₆heteroalkynyl;
   wherein said C₁₋₆alkyl, C₃₋₉cycloalkyl, C₂₋₆alkenyl, C₅₋₉cycloalkenyl, C₂₋₆alkynyl, C₅₋₉cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
   and wherein each R⁵ and R⁵ or R^{5a} and R^{5a} can be taken together in order to form a (4-, 5-, 6-, or 7-membered) heterocycle which can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O— alkyl, —OCF₃, —CHF₂, —OCHF₂, cyano, nitro, —C(O)OH; NH₂—NHalkyl, and —N(alkyl)₂;
cycle A is selected from aryl; heteroaryl; C₃₋₉cycloalkyl; and heterocycle;
   wherein said aryl, heteroaryl, C₃₋₉cycloalkyl and heterocycle is substituted with one or more R⁷;
each R⁷ is independently selected from halogen; hydroxyl; sulfhydryl; ═O; ═S; —OZ¹; —SZ¹; —SCF3; —SF₅; —CF₃; —OCF₃; —CHF₂; —OCHF₂; —NZ³Z⁴; —NZ³C(O)Z¹; cyano; —C(O)Z²; —C(O)OZ¹; —C(O)NZ³Z⁴; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylalkynyl; arylC₁₋₆ heteroalkyl; arylC₂₋₆heteroalkenyl; arylC₂₋₆heteroalkynyl; heteroarylC₁₋₆alkyl; heteroarylC₂₋₆ alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆ alkynyl; heterocycle-C₁₋₆heteroalkyl; heterocycle-C₂₋₆heteroalkenyl; and heterocycle-C₂₋₆ heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆ heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆ alkyl, —OCF₃, —CHF₂; —OCHF₂, cyano, nitro, —C(O)OH, —NH₂, —NHC₁₋₆alkyl, and —N(C₁₋₆ alkyl)₂;
X¹ is selected from CR⁸; N; and NR^{8a}; whereby X¹ can only be NR^{8a} when X² and/or X⁴ are C═O or C═S;
X² is selected from CR⁹; N; and NR^{9a}; whereby X² can only be NR^{9a} when X¹ and/or X³ are C═O or C—SH;
X³ is selected from CH; and N;
X⁴ is selected from CH; and N;
X⁵ is selected from —S(═O)₂—; —C(═O)—; and —CH₂—;
each R^{10a} is independently selected from hydrogen; and C₁₋₄alkyl;
each R^{10b} is independently selected from hydrogen; and C₁₋₄alkyl
   wherein said alkyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
   whereby maximally 2 of X¹, X², X³ and X⁴ can be a N (selected from N and NR^{8a} for X¹, from N and NR^{9a} for X², from N for X³, and from N for X⁴ respectively) at the same time;
provided that at least one of R⁸ and R⁹ is not hydrogen, each R⁸ and R⁹ are independently selected from hydrogen; halogen; hydroxyl; sulfhydryl; ═O; ═S; —OZ^{1a}; —SZ^{1a}; —SCF3; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{1a}; —NZ^{3a}C(O)Z^{1a}; —NZ^{3a}C(O)NZ^{3a}Z⁴a cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆ heteroalkenyl; arylC₂₋₆heteroalkynyl; heteroarylC₁₋₆alkyl; heteroarylC₂₋₆alkenyl; heteroarylC₂₋₆ alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆ heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each R^{8a} and R^{9a} are independently selected from hydrogen; hydroxyl; sulfhydryl; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{2a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each Z¹ and Z^{1a} is independently selected from C₁₋₆alkyl; C₃₋₉cycloakyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆heteroalkenyl; heteroarylC₂₋₆ heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆heteroalkyl, arylC₂₋₆ heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each Z² and Z^{2a} is independently selected from hydroxyl; C₁₋₆alkyl; C₃₋₉cycloakyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; C₂₋₆ heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆heteroalkyl, arylC₂₋₆ heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆ alkyl, and —N(C₁₋₆alkyl)₂;
each Z³, Z^{3a}, Z⁴, and Z^{4a} is independently selected from hydrogen; C₁₋₆alkyl; C₃₋₉cycloakyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆ alkenyl; arylC₂₋₆alkynyl; arylC₁₋₆heteroalkyl; arylC₂₋₆heteroalkenyl; arylC₂₋₆ heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₅₋₉cycloalkenyl, C₂₋₆alkynyl, C₅₋₉cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆ heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆ alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆ alkyl, and —N(C₁₋₆alkyl)₂;
and wherein each Z³ and Z⁴ or Z^{3a} and Z^{4a} can be taken together in order to form a (4-, 5-, 6-, or 7-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂.

2. A compound of formula (I), or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof,

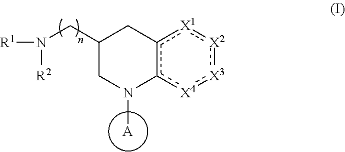

wherein:
n is selected from 0; and 1;
each — represents an optional double bond, whereby maximally 3 —are a double bond at the same time;
R¹ is selected from alkyl; cycloalkyl; heteroalkyl; —C(O)R³; —C(O)OR⁴; —C(O)NR⁵R⁶; —S(O)₂R^{3a}; —S(O)R^{4a}; —S(O)₂NR^{5a}R^{6a}; —S(O)(NR^{5a})R^{4a}; and —S(NR^{5a})(NR^{6a})R^{3a};
   wherein said alkyl, cycloalkyl and heteroalkyl can be unsubstituted or substituted with one or more substituents selected from alkyl, cycloalkyl, hydroxyl, ═O, halogen, —SH, ═S, trifluoromethyl, —O-alkyl, —OCF₃, cyano, —C(O)OH, —CONH₂, —CONHalkyl, —CON(alkyl)₂, —SO₂alkyl, —SO₂NH₂, —SO₂NHalkyl, —SO₂N(alkyl)₂, —S(O)(NH)alkyl, —S(O)(Nalkyl)alkyl, —S(NH)(NH)alkyl, —NH₂, —NHalkyl, —N(alkyl)₂;
R² is selected from hydrogen; alkyl; cycloalkyl; and heteroalkyl;
R¹ and R² can be taken together to form a (4-; 5-; or 6-membered) heterocycle which can be unsubstituted or substituted with one or more substituents selected from alkyl, cycloalkyl, hydroxyl, ═O, halogen, —SH, ═S, trifluoromethyl, —O-alkyl, —OCF₃, cyano, nitro, —C(O)OH, —CONH₂, —CONHalkyl, —CON(alkyl)₂, —SO₂alkyl, —SO₂NH₂, —SO₂NHalkyl, —SO₂N(alkyl)₂, —S(O)(NH)alkyl, —S(O)(Nalkyl)alkyl, —S(NH)(NH)alkyl, —NH₂, —NHalkyl, —N(alkyl)₂;
each R³ and R^{3a} is independently selected from hydroxyl; alkyl; cycloalkyl; alkenyl; cycloalkenyl; alkynyl; cycloalkynyl; heteroalkyl; heteroalkenyl; and heteroalkynyl;
   wherein said alkyl, cycloalkyl, alkenyl, cycloalkenyl, alkynyl, cycloalkynyl, heteroalkyl, heteroalkenyl and heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁴ and R^{4a} is independently selected from alkyl; alkenyl; cycloalkenyl; alkynyl; cycloalkyl; cycloalkynyl; heteroalkyl; heteroalkenyl; and heteroalkynyl;
   wherein said alkyl, cycloalkyl, alkenyl, cycloalkenyl, alkynyl, cycloalkynyl, heteroalkyl, heteroalkenyl and heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁵, R^{5a}, R⁶, and R^{6a} is independently selected from hydrogen; alkyl; cycloalkyl; alkenyl; cycloalkenyl; alkynyl; cycloalkynyl; heteroalkyl; heteroalkenyl; and heteroalkynyl;
   wherein said alkyl, cycloalkyl, alkenyl, cycloalkenyl, alkynyl, cycloalkynyl, heteroalkyl, heteroalkenyl and heteroalkynyl can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
   and wherein each R⁵ and R⁶ or R^{5a} and R^{6a} can be taken together in order to form a (4-, 5-, 6-, or 7-membered) heterocycle which can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂.
cycle A is selected from aryl; heteroaryl; cycloalkyl; and heterocycle;
   wherein said aryl, heteroaryl, cycloalkyl and heterocycle can be unsubstituted or substituted with one or more R⁷;
each R⁷ is independently selected from halogen; hydroxyl; sulfhydryl; ═O; ═S; —OZ¹; —SZ¹; —SCF₃; —SF₅; —CF₃; —OCF₃; —CHF₂; —OCHF₂; —NZ³Z⁴; cyano; alkyl; cycloakyl; heteroalkyl; aryl; heteroaryl; heterocycle; arylalkyl; arylheteroalkyl; heteroarylalkyl; heteroarylheteroalkyl; heterocycle-alkyl; and heterocycle-heteroalkyl;
   wherein said alkyl, cycloakyl, heteroalkyl, aryl, heteroaryl, heterocycle, arylalkyl, arylheteroalkyl, heteroarylalkyl, heteroarylheteroalkyl, heterocycle-alkyl or heterocycle-heteroalkyl can be unsubstituted or substituted with one or more substituents selected from alkyl, cycloalkyl, hydroxyl, ═O, halogen, —SH, ═S, —CF₃, —O-alkyl, —OCF₃, —CHF₂; —OCHF₂, cyano;
X¹ is selected from CR⁸; N; and NR^{8a}; whereby X¹ can only be NR^{8a} when X² and/or X⁴ are C═O or C═S;
X² is selected from CR⁹; N; and NR^{9a}; whereby X² can only be NR^{9a} when X¹ and/or X³ are C═O or C—SH;
X³ is selected from CH; and N;
X⁴ is selected from C^{H}; and N;
whereby maximally 2 of X¹, X², X³ and X⁴ can be a N (selected from N and NR^{5a} for X¹, from N and NR^{9a} for X², from N for X³, and from N for X⁴ respectively) at the same time;
each R⁸ and R⁹ are independently selected from hydrogen; halogen; hydroxyl; sulfhydryl; ═O; ═S; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; —NZ^{3a}Z^{4a}; —NZ^{3a}S(O)₂Z^{1a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; alkyl; cycloakyl; heteroalkyl; aryl; heteroaryl; heterocycle; arylalkyl; arylheteroalkyl; heteroarylalkyl; heteroarylheteroalkyl; heterocycle-alkyl; and heterocycle-heteroalkyl;
   wherein said alkyl, cycloakyl, heteroalkyl, aryl, heteroaryl, heterocycle, arylalkyl, arylheteroalkyl, heteroarylalkyl, heteroarylheteroalkyl, heterocycle-alkyl or heterocycle-heteroalkyl can be unsubstituted or substituted with one or more substituents selected from alkyl, cycloalkyl, hydroxyl, ═O, halogen, —SH, ═S, —CF₃, —O-alkyl, —OCF₃, —CHF₂; —OCHF₂, cyano, —C(O)OH, —NH₂, —NHalkyl, and —N(alkyl)₂;
each R^{8a} and R^{9a} are independently selected from hydrogen; hydroxyl; sulfhydryl; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; —NZ^{3a}Z^{4a}; —NZ^{3a}S(O)₂Z^{2a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; -alkyl; cycloakyl; heteroalkyl; aryl; heteroaryl; heterocycle; arylalkyl; arylheteroalkyl; heteroarylalkyl; heteroarylheteroalkyl; heterocycle-alkyl; and heterocycle-heteroalkyl;
   wherein said alkyl, cycloakyl, heteroalkyl, aryl, heteroaryl, heterocycle, arylalkyl, arylheteroalkyl, heteroarylalkyl, heteroarylheteroalkyl, heterocycle-alkyl or heterocycle-heteroalkyl can be unsubstituted or substituted with one or more substituents selected from alkyl, cycloalkyl, hydroxyl, ═O, halogen, —SH, ═S, —CF₃, —O-alkyl, —OCF₃, —CHF₂; —OCHF₂, cyano, —C(O)OH, —NH₂, —NHalkyl, and —N(alkyl)₂;
each Z¹ and Z^{1a} is independently selected from alkyl; alkenyl; cycloalkenyl; alkynyl; cycloalkyl; cycloalkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; or heterocycle-heteroalkynyl;
   wherein said alkyl, cycloalkyl, alkenyl, alkynyl, cycloalkyl, cycloalkenyl, cycloalkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, —C(O)OH; NH₂; —NHalkyl, and —N(alkyl)₂;
each Z² and Z^{2a} is independently selected from hydroxyl; alkyl; cycloalkyl; alkenyl; cycloalkenyl; alkynyl; cycloalkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; or heterocycle-heteroalkynyl;
   wherein said alkyl, cycloalkyl, alkenyl, cycloalkenyl, alkynyl, cycloalkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, —C(O)OH; NH₂; —NHalkyl, and —N(alkyl)₂;
each Z³, Z^{3a}, Z⁴, and Z^{4a} is independently selected from hydrogen; alkyl; cycloalkyl; alkenyl; cycloalkenyl; alkynyl; cycloalkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; or heterocycle-heteroalkynyl;
   wherein said alkyl, cycloalkyl, alkenyl, cycloalkenyl, alkynyl, cycloalkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂; and wherein each Z³ and Z⁴ or Z^{3a} and Z^{4a} can be taken together in order to form a (4-, 5-, 6-, or 7-membered) heterocycle which can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂.

3. A compound of formula (I), or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof,

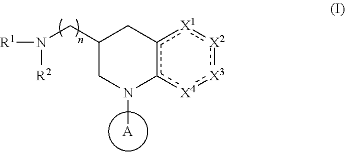

wherein:
n is selected from 0; and 1;
each — represents an optional double bond, whereby maximally 3 —are a double bond at the same time;
R¹ is selected from alkyl; —C(O)R³; —C(O)OR⁴; a n d —S(O)₂R^{3a};
   wherein said alkyl can be unsubstituted or substituted with one or more substituents selected from hydroxyl, cyano, —C(O)OH, —SO₂alkyl, —SO₂NHalkyl, —NHalkyl, —N(alkyl)₂;
R² is selected from hydrogen; alkyl; and heteroalkyl;
R¹ and R² can be taken together to form a 4-membered) heterocycle which can be unsubstituted or substituted with one or more substituents selected from alkyl, hydroxyl, ═O, —O-alkyl, cyano, —C(O)OH, —SO₂alkyl, —SO₂NHalkyl, —NHalkyl, and —N(alkyl)₂;
each R³ and R^{3a} is independently selected from alkyl; alkenyl; alkynyl; heteroalkyl; heteroalkenyl; heteroalkynyl;
   wherein said alkyl, alkenyl, alkynyl, heteroalkyl, heteroalkenyl, heteroalkynyl can be unsubstituted or substituted with one or more substituents selected from hydroxyl, ═O, halogen, —SH, ═S, —CF₃, —O-alkyl, —OCF₃, —CHF₂, —OCHF₂, cyano, —C(O)OH; NH₂; —NHalkyl, and —N(alkyl)₂;
R⁴ is selected from alkyl; alkenyl; alkynyl; heteroalkyl; heteroalkenyl; heteroalkynyl;
   wherein said alkyl, alkenyl, alkynyl, heteroalkyl, heteroalkenyl or heteroalkynyl can be unsubstituted or substituted with one or more substituents selected from hydroxyl, ═O, halogen, —SH, ═S, —CF₃, —O-alkyl, —OCF₃, —CHF₂, —OCHF₂, cyano, —C(O)OH; NH₂; —NHalkyl, and —N(alkyl)₂;
cycle A is selected from aryl; heteroaryl; and cycloalkyl;
   wherein said aryl, heteroaryl, cycloalkyl and heterocycle can be unsubstituted or substituted with one or more R⁷;
each R⁷ is independently selected from halogen; —OZ¹; —SCF3; —SF₅; —CF₃; —OCF₃; —CHF₂; —OCHF₂; alkyl; cycloakyl; heteroalkyl; aryl;
   wherein said alkyl, cycloakyl, heteroalkyl and aryl can be unsubstituted or substituted with one or more substituents selected from alkyl, hydroxyl, halogen, —SH, —CF₃, —O— alkyl, —OCF₃, —CHF₂; —OCHF₂, cyano;
X¹ is selected from CR⁸; N; and NR^{8a}; whereby X¹ can only be NR^{8a} when X² and/or X⁴ are C—OH or C—SH;
X² is selected from CR⁹; and N;
X³ is selected from CH; and N;
X⁴ is selected from CH; and N;
whereby maximally 2 of X¹, X², X³ and X⁴ can be a N (selected from N and NR^{8a} for X¹, from N for X², from N for X³, and from N for X⁴ respectively) at the same time;
each R⁸ and R⁹ are independently selected from hydrogen; halogen; hydroxyl; sulfhydryl; ═O; ═S; —OZ^{1a}; —CF₃; —OCF₃; —CHF₂; —OCHF₂; —NZ^{3a}Z^{4a}; —NZ^{3a}S(O)₂Z^{1a}; —NZ^{3a}C(O)Z^{1a}; cyano; —C(O)OZ^{1a}; —C(O)NZ^{3a}Z^{4a}; alkyl; heteroalkyl; aryl; heteroaryl; heterocycle; arylalkyl; heteroarylalkyl; and heterocycle-alkyl;
   wherein said alkyl, heteroalkyl, aryl, heteroaryl, heterocycle, arylalkyl, heteroarylalkyl or heterocycle-alkyl can be unsubstituted or substituted with one or more substituents selected from alkyl, hydroxyl, halogen, —CF₃, —O-alkyl, —OCF₃, —CHF₂; —OCHF₂, cyano, —NH₂;
R^{8a} is independently selected from hydrogen; alkyl; heteroalkyl; aryl; heteroaryl; heterocycle; arylalkyl; heteroarylalkyl; and heterocycle-alkyl;
   wherein said alkyl, heteroalkyl, aryl, heteroaryl, heterocycle, arylalkyl, heteroarylalkyl or heterocycle-alkyl can be unsubstituted or substituted with one or more substituents selected from alkyl, hydroxyl, halogen, —CF₃, —O-alkyl, —OCF₃, —CHF₂; —OCHF₂, cyano, —NH₂;
each Z¹ and Z^{1a} is independently selected from alkyl; alkenyl; alkynyl; heteroalkyl; heteroalkenyl; heteroalkynyl;
   wherein said alkyl, alkenyl, alkynyl, heteroalkyl, heteroalkenyl or heteroalkynyl can be unsubstituted or substituted with one or more substituents selected from hydroxyl, ═O, halogen, —SH, ═S, —CF₃, —O-alkyl, —OCF₃, —CHF₂, —OCHF₂, cyano, —C(O)OH; NH₂; —NHalkyl, and —N(alkyl)₂;
each Z^{3a} and Z^{4a} is independently selected from hydrogen; alkyl; alkenyl; alkynyl; heteroalkyl; heteroalkenyl; heteroalkynyl; aryl; heteroaryl; heterocycle;
   wherein said alkyl, alkenyl, alkynyl, heteroalkyl, heteroalkenyl, heteroalkynyl, aryl, heteroaryl and heterocycle can be unsubstituted or substituted with one or more substituents selected from hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;

4. A compound of formula (I), or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof,

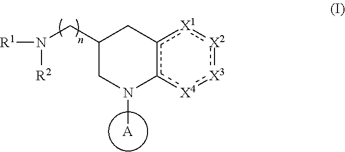

wherein
n, ——, R¹, R², cycle A, R⁷, X¹, X², X³, X⁴, R^{8e}, R⁴, Z¹, Zia, R³, R^{3a}, Z^{3a} and Z^{4a} have the same meaning as in statements 1, 2, or 3, or other embodiments, statements or aspects described herein;
R⁸ is selected from hydrogen; halogen; hydroxyl; sulfhydryl; ═O; ═S; —OZ^{1a}; —CF₃; —OCF₃; —CHF₂; —OCHF₂; —NZ^{3a}Z^{4a}; —NZ^{3a}S(O)₂Z^{1a}; —NZ^{3a}C(O)Z^{1a}; cyano; —C(O)OZ^{1a}; —C(O)NZ^{3a}Z^{4a}; alkyl; heteroalkyl; aryl; heteroaryl; heterocycle; arylalkyl; heteroarylalkyl; and heterocycle-alkyl;
   wherein said alkyl, heteroalkyl, aryl, heteroaryl, heterocycle, arylalkyl, heteroarylalkyl or heterocycle-alkyl can be unsubstituted or substituted with one or more substituents selected from alkyl, hydroxyl, halogen, —CF₃, —O-alkyl, —OCF₃, —CHF₂; —OCHF₂, cyano, —NH₂;
R⁹ is selected from hydrogen; halogen; hydroxyl; sulfhydryl; ═O; ═S; —OZ^{1a}; —CF₃; —OCF₃; —CHF₂; —OCHF₂; —NZ^{3a}Z^{4a}; alkyl; and heteroalkyl;
wherein said alkyl and heteroalkyl can be unsubstituted or substituted with one or more substituents selected from hydroxyl, halogen, —CF₃, —O-alkyl, —OCF₃, —CHF₂; —OCHF₂, cyano, —NH₂.

In a specific embodiment of the present disclosure, the compounds have a structure according to formula (Ia) or formula (I) described herein, more in particular according to the other formulas, statements, embodiments and aspects described herein, yet more in particular according to statements 1, 2, 3, and 4 herein, whereby:
each alkyl is C₁-C₁₈ membered alkyl, more in particular is a C₁-C₁₂ membered alkyl; yet more in particular is a C₁-C₉ membered alkyl; still more in particular is a C₁—C membered alkyl; including when such alkyl is linked for example to aryl, heteroaryl or heterocycle as for example in arylalkyl, heteroarylalkyl and heterocycle-alkyl;
each alkenyl is C₂-C₁₈ membered alkenyl, more in particular is a C₂-C₁₂ membered alkenyl; yet more in particular is a C₂-C₉ membered alkenyl; still more in particular is a C₂-C₆ membered alkenyl; including when such alkenyl is linked to for example aryl, heteroaryl or heterocycle as for example in arylalkenyl, heteroarylalkenyl and heterocycle-alkenyl;
each alkynyl is C₂-C₁₈ membered alkynyl, more in particular is a C₂-C₁₂ membered alkynyl; yet more in particular is a C₂-C₉ membered alkynyl; still more in particular is a C₂-C₆ membered alkynyl; including when such alkynyl is linked to for example aryl, heteroaryl or heterocycle as for example in arylalkynyl, heteroarylalkynyl and heterocycle-alkynyl;
each heteroalkyl is C₁-C₁₈ membered alkyl, more in particular is a C₁-C₁₂ membered heteroalkyl; yet more in particular is a C₁—C membered heteroalkyl; still more in particular is a C₁—C membered heteroalkyl; including when such heteroalkyl is linked for example to aryl, heteroaryl or heterocycle as for example in arylheteroalkyl, heteroarylheteroalkyl and heterocycle-heteroalkyl;
each heteroalkenyl is C₂-C₁₈ membered alkenyl, more in particular is a C₂-C₁₂ membered heteroalkenyl; yet more in particular is a C₂-C₉ membered heteroalkenyl; still more in particular is a C₂-C₆ membered heteroalkenyl; including when such heteroalkenyl is linked to for example aryl, heteroaryl or heterocycle as for example in arylheteroalkenyl, heteroarylheteroalkenyl and heterocycle-heteroalkenyl; and
each heteroalkynyl is C₂-C₁₈ membered alkynyl, more in particular is a C₂-C₁₂ membered heteroalkynyl; yet more in particular is a C₂-C₉ membered heteroalkynyl; still more in particular is a C₂-C₆ membered heteroalkynyl; including when such alkenyl is linked to for example aryl, heteroaryl or heterocycle as for example in arylheteroalkynyl, heteroarylheteroalkynyl and heterocycle-heteroalkynyl.

5. A compound of formula (I), or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof,

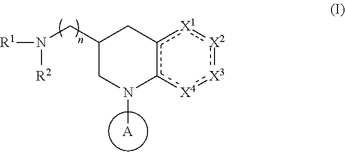

wherein:
n is selected from 0; 1; and 2;
each — represents an optional double bond, whereby maximally 3 —are a double bond at the same time;
R¹ is selected from C₁₋₆alkyl; C₃₋₉cycloalkyl; C₂₋₆alkenyl; C₅₋₉cycloalkenyl; C₂₋₆alkynyl; C₅₋₉ cycloalkynyl; C₁₋₆heteroalkyl; C₂₋₆heteroalkenyl; C₂₋₆heteroalkynyl; —C(O)H; —C(O)R³; —C(O)OR⁴; —C(O)NR⁵R⁶; —S(O)₂R^{3a}; —S(O)R^{4a}; —S(O)₂NR^{5a}R^{6a}; —S(O)(NR^{5a})R^{4a}; —S(NR^{5a})(NR^{6a})R^{3a}; and —P(O)R^{5b}R^{6b};
   wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from C₁₋₆alkyl, C₃₋₉ cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆ alkyl)₂, —S(O)(NH)C₁₋₆alkyl, —S(O)(NC₁₋₆alkyl)C₁₋₆alkyl, —S(NH)(NH)C₁₋₆alkyl, —S(O)(NH)C₁₋₆alkyl, —S(O)(NC₁₋₆alkyl)C₁₋₆alkyl, —S(NH)(NH)C₁₋₆alkyl, —NH₂, —NHC₁₋₆ alkyl, —N(C₁₋₆alkyl)₂;
R² is selected from hydrogen; C₁₋₆alkyl; C₃₋₉cycloalkyl; and C₁₋₆heteroalkyl;
R¹ and R² can be taken together to form a (4-; 5-; 6- or 7-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆ alkyl)₂, —S(O)(NH)C₁₋₆alkyl, —S(O)(NC₁₋₆alkyl)C₁₋₆alkyl, —S(NH)(NH)C₁₋₆alkyl, —NH₂, —NHC₁₋₆alkyl, —N(C₁₋₆alkyl)₂;
each R³ and R^{3a} is independently selected from hydroxyl; C₁₋₆alkyl; C₃₋₉cycloalkyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; and C₂₋₆ heteroalkynyl;
   wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁴ and R^{4a} is independently selected from C₁₋₆alkyl; C₃₋₉cycloalkyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; and C₂₋₆ heteroalkynyl;
   wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁵, R⁵, R^{5b}, R⁶, R^{6a} and R^{6b} is independently selected from hydrogen; C₁₋₆alkyl; C₃₋₉cycloalkyl; C₂₋₆alkenyl; C₅₋₉cycloalkenyl; C₂₋₆alkynyl; C₅₋₉cycloalkynyl; C₁₋₆heteroalkyl; C₂₋₆ heteroalkenyl; and C₂₋₆heteroalkynyl;
   wherein said C₁₋₆alkyl, C₃₋₉cycloalkyl, C₂₋₆alkenyl, C₅₋₉cycloalkenyl, C₂₋₆alkynyl, C₅₋₉cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
   and wherein each R⁵ and R⁶ or R^{5a} and R^{6a} can be taken together in order to form a (4-, 5-, 6-, or 7-membered) heterocycle which can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O— alkyl, —OCF₃, —CHF₂, —OCHF₂, cyano, nitro, —C(O)OH; NH₂. —NHalkyl, and —N(alkyl)₂;
cycle A is selected from aryl; heteroaryl; C₃₋₉cycloalkyl; and heterocycle;
   wherein said aryl, heteroaryl, C₃₋₉cycloalkyl and heterocycle can be unsubstituted or substituted with one or more R⁷;
each R⁷ is independently selected from halogen; hydroxyl; sulfhydryl; ═O; ═S; —OZ¹; —SZ¹; —SCF₃; —SF₅; —CF₃; —OCF₃; —CHF₂; —OCHF₂; —NZ³Z⁴; —NZ³C(O)Z¹; cyano; —C(O)Z²; —C(O)OZ¹; —C(O)NZ³Z⁴; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylalkynyl; arylC₁₋₆ heteroalkyl; arylC₂₋₆heteroalkenyl; arylC₂₋₆heteroalkynyl; heteroarylC₁₋₆alkyl; heteroarylC₂₋₆ alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆ alkynyl; heterocycle-C₁₋₆heteroalkyl; heterocycle-C₂₋₆heteroalkenyl; and heterocycle-C₂₋₆ heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆ heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆ alkyl, —OCF₃, —CHF₂; —OCHF₂, cyano, nitro, —C(O)OH, —NH₂, —NHC₁₋₆alkyl, and —N(C₁₋₆ alkyl)₂;
X¹ is selected from CR⁸; N; and NR^{8a}; whereby X¹ can only be NR^{8a} when X² and/or X⁴ are C═O or C═S;
X² is selected from CR⁹; N; and NR^{9a}; whereby X² can only be NR^{9a} when X¹ and/or X³ are C═O or C—SH;
X³ is selected from CH; and N;
X⁴ is selected from CH; and N;
whereby maximally 2 of X¹, X², X³ and X⁴ can be a N (selected from N and NR^{8a} for X¹, from N and NR^{9a} for X², from N for X³, and from N for X⁴ respectively) at the same time;
each R⁸ and R⁹ are independently selected from hydrogen; halogen; hydroxyl; sulfhydryl; ═O; ═S; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{1a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each R^{8a} and R^{9a} are independently selected from hydrogen; hydroxyl; sulfhydryl; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{2a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each Z¹ and Z^{1a} is independently selected from C₁₋₆alkyl; C₃₋₉cycloakyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; C₂₋₆ heteroalkynyl; aryl; heteroaryl; heterocycle; arylC₁₋₆alkyl; arylC₂₋₆alkenyl; arylC₂₋₆alkynyl; arylC₁₋₆ heteroalkyl; arylC₂₋₆heteroalkenyl; arylC₂₋₆heteroalkynyl; heteroarylC₁₋₆alkyl; heteroarylC₂₋₆ alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆ alkynyl; heterocycle-C₁₋₆heteroalkyl; heterocycle-C₂₋₆heteroalkenyl; and heterocycle-C₂₋₆ heteroalkynyl;
   wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆heteroalkyl, arylC₂₋₆ heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each Z² and Z^{2a} is independently selected from hydroxyl; C₁₋₆alkyl; C₃₋₉cycloakyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; C₂₋₆ heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆heteroalkyl, arylC₂₋₆ heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂;
each Z³, Z^{3a}, Z⁴, and Z^{4a} is independently selected from hydrogen; C₁₋₆alkyl; C₃₋₉cycloakyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆ alkenyl; arylC₂₋₆alkynyl; arylC₁₋₆heteroalkyl; arylC₂₋₆heteroalkenyl; arylC₂₋₆ heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₅₋₉cycloalkenyl, C₂₋₆alkynyl, C₅₋₉cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆ heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆ alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆ alkyl, and —N(C₁₋₆alkyl)₂;
   and wherein each Z³ and Z⁴ or Z^{3a} and Z^{4a} can be taken together in order to form a (4-, 5-, 6-, or 7-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂.

In a specific embodiment of the present disclosure, the compounds have a structure according to formula I described herein, more in particular according to the formulas, statements, embodiments and aspects described herein, yet more in particular according to statements 1, 2, 3, 4, and 5 herein, whereby:
each aryl is C₆-C₂₀ membered aryl, more in particular is a C₆-C₁₄ membered aryl; yet more in particular is a C₆-C₁₀ membered aryl; including when such aryl is linked to alkyl, alkenyl, alkynyl, heteroalkyl, heteroalkenyl or heteroalkynyl, as in arylalkyl, arylalkenyl, arylalkynyl, arylheteroalkyl, arylheteroalkenyl, arylheteroalkynyl;
each heteroaryl is 5 to 20 membered heteroaryl, more in particular is a 6 to 14 membered heteroaryl; yet more in particular is a 6 to 10 membered heteroaryl; including when such heteroaryl is linked to alkyl, alkenyl or alkynyl such as in heteroarylalkyl, heteroarylalkenyl, heteroarylalkynyl, heteroarylheteroalkyl, heteroarylheteroalkenyl, heteroarylheteroalkynyl;
each heterocycle is 5 to 20 membered heterocycle, more in particular is a 6 to 14 membered heterocycle; yet more in particular is a 6 to 10 membered heterocycle; including when such heterocycle is linked to alkyl, alkenyl, alkynyl, heteroalkyl, heteroalkenyl and heteroalkynyl as in heterocyclealkyl, heterocyclealkenyl, heterocyclealkynyl, heterocycleheteroalkyl, heterocycleheteroalkenyl and heterocycleheteroalkynyl.

6. A compound of formula (I), or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof,

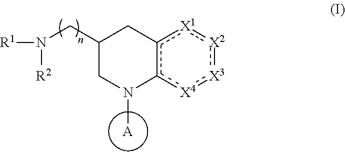

wherein:
n is selected from 0; and 1;
each — represents an optional double bond, whereby maximally 3 —are a double bond at the same time;
R¹ is selected from C₁₋₆alkyl; —C(O)R³; —C(O)OR⁴; and —S(O)₂R^{3a};
   wherein said C₁₋₆alkyl can be unsubstituted or substituted with one or more substituents selected from hydroxyl, cyano, —C(O)OH, —SO₂C₁₋₆alkyl, —SO₂NHC₁₋₆alkyl, —NHC₁₋₆alkyl, —N(C₁₋₆alkyl)₂;
R² is selected from hydrogen; C₁₋₆alkyl; and C₁₋₆heteroalkyl;
R¹ and R² can be taken together to form a 4-membered) heterocycle which can be unsubstituted or substituted with one or more substituents selected from C₁₋₆alkyl, hydroxyl, ═O, —O—C₁₋₆alkyl, cyano, —C(O)OH, —SO₂C₁₋₆alkyl, —SO₂NHC₁₋₆alkyl, —NHC₁₋₆alkyl, and —N(C₁₋₆ alkyl)₂;
each R³ and R^{3a} is independently selected from C₁₋₆alkyl; C₂₋₆alkenyl; C₂₋₆alkynyl; C₁₋₆ heteroalkyl; C₂₋₆heteroalkenyl; C₂₋₆heteroalkynyl;
   wherein said C₁₋₆alkyl, C₂₋₆alkenyl, C₂₋₆alkynyl, C₁₋₆heteroalkyl, C₂₋₆heteroalkenyl or C₂₋₆ heteroalkynyl can be unsubstituted or substituted with one or more substituents selected from hydroxyl, ═O, halogen, —SH, ═S, —CF₃, —O— C₁₋₆alkyl, —OCF₃, —CHF₂, —OCHF₂, cyano, —C(O)OH; NH₂; —NHC₁₋₆alkyl, and —N(C₁₋₆alkyl)₂;
R⁴ is independently selected from C₁₋₆alkyl; C₂₋₆alkenyl; C₂₋₆alkynyl; C₁₋₆heteroalkyl; C₂₋₆ heteroalkenyl; and C₂₋₆heteroalkynyl;
   wherein said C₁₋₆alkyl, C₂₋₆alkenyl, C₂₋₆alkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
cycle A is selected from aryl; heteroaryl; and C₃₋₉cycloalkyl;
   wherein said aryl, heteroaryl, C₃₋₉cycloalkyl and heterocycle can be unsubstituted or substituted with one or more R⁷;
each R⁷ is independently selected from halogen; —OZ¹; —SCF₃; —SF₅; —CF₃; —OCF₃; —CHF₂; —OCHF₂; C₁₋₆alkyl; C₃₋₉cycloakyl; heteroalkyl; aryl;
   wherein said C₁₋₆alkyl, C₃₋₉cycloakyl, C₁₋₆heteroalkyl and aryl can be unsubstituted or substituted with one or more substituents selected from C₁₋₆alkyl, hydroxyl, halogen, —SH, —CF₃, —O—C₁₋₆alkyl, —OCF₃, —CHF₂; —OCHF₂, cyano;
X¹ is selected from CR⁸; N; and NR^{8a}; whereby X¹ can only be NR^{8a} when X² and/or X⁴ are C—OH or C—SH;
X² is selected from CR⁹; and N;
X³ is selected from CH; and N;
X⁴ is selected from CH; and N;
whereby maximally 2 of X¹, X², X³ and X⁴ can be a N (selected from N and NR^{8a} for X¹, from N for X², from N for X³, and from N for X⁴ respectively) at the same time;
R⁸ is selected from hydrogen; halogen; hydroxyl; sulfhydryl; ═O; ═S; —OZ^{1a}; —CF₃; —OCF₃; —CHF₂: —OCHF₂: —NZ^{3a}Z⁴a: —NZ^{3a}S(O)₂Z^{1a}; —NZ^{3a}C(O)Z^{1a}; cyano; —C(O)OZ^{1a}: —C(O)NZ^{3a}Z^{4a}; C₁₋₆alkyl; C₁₋₆heteroalkyl; aryl; heteroaryl; heterocycle; arylC₁₋₆alkyl; heteroarylC₁₋₆alkyl; and heterocycle-C₁₋₆alkyl;
   wherein said C₁₋₆alkyl, C₁₋₆heteroalkyl, aryl, heteroaryl, heterocycle, arylC₁₋₆alkyl, heteroarylC₁₋₆alkyl or heterocycle-C₁₋₆alkyl can be unsubstituted or substituted with one or more substituents selected from alkyl, hydroxyl, halogen, —CF₃, —O—C₁₋₆alkyl, —OCF₃, —CHF₂; —OCHF₂, cyano, —NH₂;
R⁹ is selected from hydrogen; halogen; hydroxyl; sulfhydryl; ═O; ═S; —OZ^{1a}; —CF₃; —OCF₃; —CHF₂; —OCHF₂; —NZ^{3a}Z^{4a}; C₁₋₆alkyl; and C₁₋₆heteroalkyl;
wherein said C₁₋₆alkyl and C₁₋₆heteroalkyl can be unsubstituted or substituted with one or more substituents selected from hydroxyl, halogen, —CF₃, —O—C₁₋₆alkyl, —OCF₃, —CHF₂; —OCHF₂, cyano, —NH₂; —R^{8a} is independently selected from hydrogen; C₁₋₆alkyl; heteroalkyl; aryl; heteroaryl; heterocycle; arylC₁₋₆alkyl; heteroarylC₁₋₆alkyl; and heterocycle-C₁₋₆alkyl;
   wherein said C₁₋₆alkyl, heteroalkyl, aryl, heteroaryl, heterocycle, arylC₁₋₆alkyl, heteroarylC₁₋₆alkyl or heterocycle-C₁₋₆alkyl can be unsubstituted or substituted with one or more substituents selected from C₁₋₆alkyl, hydroxyl, halogen, —CF₃, —O—C₁₋₆alkyl, —OCF₃, —CHF₂; —OCHF₂, cyano, —NH₂,
each Z¹ and Z^{1a} is independently selected from C₁₋₆alkyl; C₂₋₆alkenyl; C₂₋₆alkynyl; C₁₋₆ heteroalkyl; C₂₋₆heteroalkenyl; C₂₋₆heteroalkynyl;
   wherein said C₁₋₆alkyl, C₂₋₆alkenyl, C₂₋₆alkynyl, C₁₋₆heteroalkyl, C₂₋₆heteroalkenyl or C₂₋₆ heteroalkynyl can be unsubstituted or substituted with one or more substituents selected from hydroxyl, ═O, halogen, —SH, ═S, —CF₃, —O— C₁₋₆alkyl, —OCF₃, —CHF₂, —OCHF₂, cyano, —C(O)OH; NH₂; —NHC₁₋₆alkyl, and —N(C₁₋₆alkyl)₂;
each Z^{3a} and Z^{4a} is independently selected from hydrogen; C₁₋₆alkyl; C₂₋₆alkenyl; C₂₋₆ alkynyl; C₁₋₆heteroalkyl; C₂₋₆heteroalkenyl; C₂₋₆heteroalkynyl; aryl; heteroaryl; heterocycle;
   wherein said C₁₋₆alkyl, C₂₋₆alkenyl, C₂₋₆alkynyl, C₁₋₆heteroalkyl, C₂₋₆ heteroalkenyl, C₂₋₆heteroalkynyl, aryl, heteroaryl and heterocycle can be unsubstituted or substituted with one or more substituents selected from hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂.

7. A compound of formula (I), or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof,

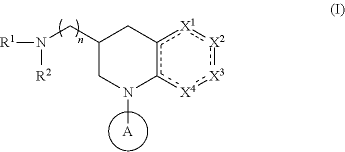

wherein
each of n, —, R¹, R², R⁷, X¹, X², X³, and X⁴ have the same meaning as defined herein in the different subgroups, statements, aspects or embodiments described herein;
cycle A is selected from unsubstituted or substituted with one or more R⁷ phenyl; naphthalenyl;
anthracenyl; cyclopropyl; cyclobutyl; cyclopentyl; cyclohexyl; cycloheptyl; cyclooctyl; norbornyl;
fenchyl; decalinyl; adamantly; triazolyl; pyridinyl; pyrazolyl; pyrrolyl; furanyl; thiophenyl;
imidazolyl; oxazolyl; isoxazolyl; thiazolyl; isothiazolyl; oxadiazolyl; thiadiazolyl; tetrazolyl;
oxatriazolyl; pyrimidyl; pyrazinyl; pyridazinyl; triazinyl; indolyl; indolizinyl; isoindolyl;
benzofuranyl; benzothiophenyl; indazolyl; benzimidazolyl; benzoxazolyl; benzisoxazolyl;
benzothiazolyl; benzoisothiazolyl; dihydro-benzofuranyl; thienopyridinyl; imidazopyridinyl;
benzodioxolyl; quinolinyl; isoquinolinyl; cinnolinyl; quinazolinyl; and quinoxalinyl.

8. A compound, or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof, selected from one or more of the compounds of Table 1.

9. A compound of formula (I), or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof,

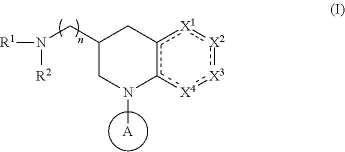

wherein:
n is selected from 0; and 1;
each — represents an optional double bond, whereby maximally 3 —are a double bond at the same time;
R¹ is selected from C₁₋₆alkyl; —C(O)R³; —C(O)OR⁴; and —S(O)₂R^{3a};
   wherein said C₁₋₆alkyl can be unsubstituted or substituted with one or more substituents selected from hydroxyl, cyano, —C(O)OH, —SO₂C₁₋₆alkyl, —SO₂NHC₁₋₆alkyl, —NHC₁₋₆alkyl, —N(C₁₋₆alkyl)₂;
R² is selected from hydrogen; C₁₋₆alkyl; and C₁₋₆heteroalkyl;
R¹ and R² can be taken together to form a 4-membered) heterocycle which can be unsubstituted or substituted with one or more substituents selected from C₁₋₆alkyl, hydroxyl, ═O, —O—C₁₋₆alkyl, cyano, —C(O)OH, —SO₂C₁₋₆alkyl, —SO₂NHC₁₋₆alkyl, —NHC₁₋₆alkyl, and —N(C₁₋₆ alkyl)₂;
each R³ and R^{3a} is independently selected from C₁₋₆alkyl; C₂₋₆alkenyl; C₂₋₆alkynyl; C₁₋₆ heteroalkyl; C₂₋₆heteroalkenyl; C₂₋₆heteroalkynyl;
   wherein said C₁₋₆alkyl, C₂₋₆alkenyl, C₂₋₆alkynyl, C₁₋₆heteroalkyl, C₂₋₆heteroalkenyl or C₂₋₆ heteroalkynyl can be unsubstituted or substituted with one or more substituents selected from hydroxyl, ═O, halogen, —SH, ═S, —CF₃, —O— C₁₋₆alkyl, —OCF₃, —CHF₂, —OCHF₂, cyano, —C(O)OH; NH₂; —NHC₁₋₆alkyl, and —N(C₁₋₆alkyl)₂;
R⁴ is independently selected from C₁₋₆alkyl; C₂₋₆alkenyl; C₂₋₆alkynyl; C₁₋₆heteroalkyl; C₂₋₆ heteroalkenyl; and C₂₋₆heteroalkynyl;
   wherein said C₁₋₆alkyl, C₂₋₆alkenyl, C₂₋₆alkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
cycle A is selected from phenyl; cyclopentyl; cyclohexyl; cycloheptyl; pyridinyl; pyrimidyl; pyrazinyl; pyridazinyl; indolyl; indolizinyl; isoindolyl; indazolyl; benzimidazolyl; benzoxazolyl; benzisoxazolyl; benzothiazolyl; benzoisothiazolyl; thienopyridinyl; imidazopyridinyl; quinolinyl; isoquinolinyl; cinnolinyl; quinazolinyl; and quinoxalinyl;
   wherein said phenyl, cyclopentyl, cyclohexyl, cycloheptyl, pyridinyl, pyrimidyl, pyrazinyl, pyridazinyl, indolyl, indolizinyl, isoindolyl, indazolyl, benzimidazolyl, benzoxazolyl, benzisoxazolyl, benzothiazolyl, benzoisothiazolyl, thienopyridinyl, imidazopyridinyl, quinolinyl, isoquinolinyl, cinnolinyl, quinazolinyl, and quinoxalinyl can be unsubstituted or substituted with one or more R⁷;
each R⁷ is independently selected from halogen; —OZ¹; —SCF₃; —SF₅; —CF₃; —OCF₃; —CHF₂; —OCHF₂; C₁₋₆alkyl; C₃₋₉cycloakyl; heteroalkyl; aryl;
   wherein said C₁₋₆alkyl, C₃₋₉cycloakyl, C₁₋₆heteroalkyl and aryl can be unsubstituted or substituted with one or more substituents selected from C₁₋₆alkyl, hydroxyl, halogen, —SH, —CF₃, —O—C₁₋₆alkyl, —OCF₃, —CHF₂; —OCHF₂, cyano;
X¹ is selected from CR⁸; N; and NR^{8a}; whereby X¹ can only be NR^{8a} when X² and/or X⁴ are C—OH or C—SH;
X² is selected from CR⁹; and N;
X³ is selected from CH; and N;
X⁴ is selected from CH; and N;
whereby maximally 2 of X¹, X², X³ and X⁴ can be a N (selected from N and NR^{8a} for X¹, from N for X², from N for X³, and from N for X⁴ respectively) at the same time;
R⁸ is selected from hydrogen; halogen; hydroxyl; sulfhydryl; ═O; ═S; —OZ^{1a}; —CF₃; —OCF₃; —CHF₂; —OCHF₂; —NZ^{3a}Z^{4a}; —NZ^{3a}S(O)₂Z^{1a}; —NZ^{3a}C(O)Z^{1a}; cyano; —C(O)OZ^{1a}; —C(O)NZ^{3a}Z^{4a}; C₁₋₆alkyl; C₁₋₆heteroalkyl; aryl; heteroaryl; heterocycle; arylC₁₋₆alkyl; heteroarylC₁₋₆alkyl; and heterocycle-C₁₋₆alkyl;
   wherein said C₁₋₆alkyl, C₁₋₆heteroalkyl, aryl, heteroaryl, heterocycle, arylC₁₋₆alkyl, heteroarylC₁₋₆alkyl or heterocycle-C₁₋₆alkyl can be unsubstituted or substituted with one or more substituents selected from alkyl, hydroxyl, halogen, —CF₃, —O—C₁₋₆alkyl, —OCF₃, —CHF₂; —OCHF₂, cyano, —NH₂;
R⁹ is selected from hydrogen; halogen; hydroxyl; sulfhydryl; ═O; ═S; —OZ^{1a}; —CF₃; —OCF₃; —CHF₂; —OCHF₂; —NZ^{3a}Z^{4a}; C₁₋₆alkyl; and C₁₋₆heteroalkyl;
wherein said C₁₋₆alkyl and C₁₋₆heteroalkyl can be unsubstituted or substituted with one or more substituents selected from hydroxyl, halogen, —CF₃, —O—C₁₋₆alkyl, —OCF₃, —CHF₂; —OCHF₂, cyano, —NH₂; —R^{8a} is independently selected from hydrogen; C₁₋₆alkyl; heteroalkyl; aryl; heteroaryl; heterocycle; arylC₁₋₆alkyl; heteroarylC₁₋₆alkyl; and heterocycle-C₁₋₆alkyl;
   wherein said C₁₋₆alkyl, heteroalkyl, aryl, heteroaryl, heterocycle, arylC₁₋₆alkyl, heteroarylC₁₋₆alkyl or heterocycle-C₁₋₆alkyl can be unsubstituted or substituted with one or more substituents selected from C₁₋₆alkyl, hydroxyl, halogen, —CF₃, —O—C₁₋₆alkyl, —OCF₃, —CHF₂; —OCHF₂, cyano, —NH₂,
each Z¹ and Z^{1a} is independently selected from C₁₋₆alkyl; C₂₋₆alkenyl; C₂₋₆alkynyl; C₁₋₆ heteroalkyl; C₂₋₆heteroalkenyl; C₂₋₆heteroalkynyl;
   wherein said C₁₋₆alkyl, C₂₋₆alkenyl, C₂₋₆alkynyl, C₁₋₆heteroalkyl, C₂₋₆heteroalkenyl or C₂₋₆ heteroalkynyl can be unsubstituted or substituted with one or more substituents selected from hydroxyl, ═O, halogen, —SH, ═S, —CF₃, —O— C₁₋₆alkyl, —OCF₃, —CHF₂, —OCHF₂, cyano, —C(O)OH; NH₂; —NHC₁₋₆alkyl, and —N(C₁₋₆alkyl)₂;
each Z^{3a} and Z^{4a} is independently selected from hydrogen; C₁₋₆alkyl; C₂₋₆alkenyl; C₂₋₆ alkynyl; C₁₋₆heteroalkyl; C₂₋₆heteroalkenyl; C₂₋₆heteroalkynyl; aryl; heteroaryl; heterocycle;
   wherein said C₁₋₆alkyl, C₂₋₆alkenyl, C₂₋₆alkynyl, C₁₋₆heteroalkyl, C₂₋₆ heteroalkenyl, C₂₋₆heteroalkynyl, aryl, heteroaryl and heterocycle can be unsubstituted or substituted with one or more substituents selected from hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂.

10. A compound of formulas (II), (IIa), (IIb), (IIc), (IId), (IIe), and (IIf) or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof,

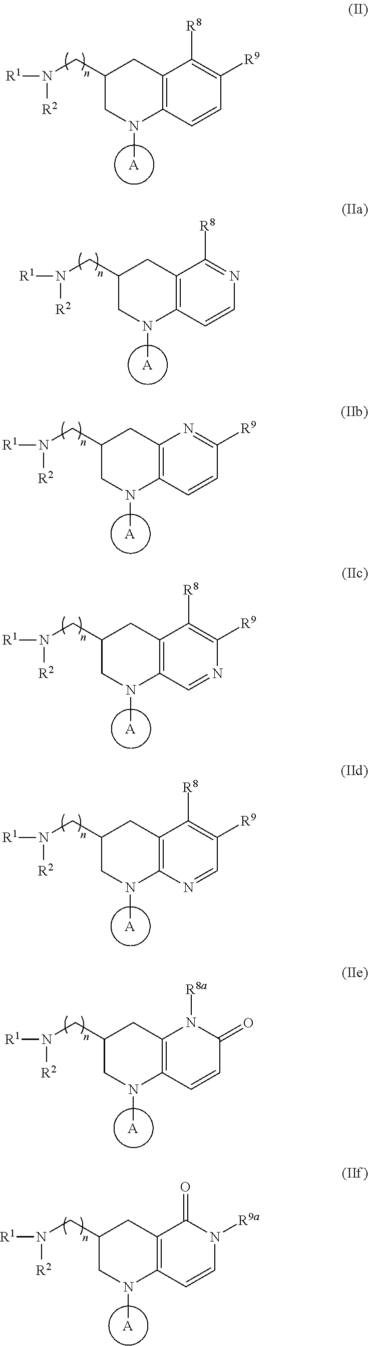

wherein:
n is selected from 0; and 1;
R¹ is selected from alkyl; cycloalkyl; heteroalkyl; —C(O)R³; —C(O)OR⁴; —S(O)₂R^{3a}; —S(O)R^{4a}; —S(O)₂NR^{5a}R^{6a}; —S(O)(NR^{5a})R^{4a}; and —S(NR^{5a})(NR^{6a})R^{3a};
   wherein said alkyl, cycloalkyl and heteroalkyl can be unsubstituted or substituted with one or more substituents selected from alkyl, cycloalkyl, hydroxyl, ═O, halogen, —SH, ═S, trifluoromethyl, —O-alkyl, —OCF₃, cyano, —C(O)OH, —CONH₂, —CONHalkyl, —CON(alkyl)₂, —SO₂alkyl, —SO₂NH₂, —SO₂NHalkyl, —SO₂N(alkyl)₂, —S(O)(NH)alkyl, —S(O)(Nalkyl)alkyl, —S(NH)(NH)alkyl, —NH₂, —NHalkyl, —N(alkyl)₂;
R² is selected from hydrogen; alkyl; cycloalkyl; and heteroalkyl;
R¹ and R² can be taken together to form a (4-; 5-; or 6-membered) heterocycle which can be unsubstituted or substituted with one or more substituents selected from alkyl, cycloalkyl, hydroxyl, ═O, halogen, —SH, ═S, trifluoromethyl, —O-alkyl, —OCF₃, cyano, nitro, —C(O)OH, —CONH₂, —CONHalkyl, —CON(alkyl)₂, —SO₂alkyl, —SO₂NH₂, —SO₂NHalkyl, —SO₂N(alkyl)₂, —S(O)(NH)alkyl, —S(O)(Nalkyl)alkyl, —S(NH)(NH)alkyl, —NH₂, —NHalkyl, —N(alkyl)₂;
each R³ and R^{3a} is independently selected from hydroxyl; C₁₋₆alkyl; C₃₋₉cycloalkyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; and C₂₋₆ heteroalkynyl;
   wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁴ and R^{4a} is independently selected from C₁₋₆alkyl; C₃₋₉cycloalkyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; and C₂₋₆ heteroalkynyl;
   wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁵, R⁵, R^{5b}, R⁵, R^{5a} and R^{5b} is independently selected from hydrogen; C₁₋₆alkyl; C₃₋₉cycloalkyl; C₂₋₆alkenyl; C₅₋₉cycloalkenyl; C₂₋₆alkynyl; C₅₋₉cycloalkynyl; C₁₋₆heteroalkyl; C₂₋₆ heteroalkenyl; and C₂₋₆heteroalkynyl;
   wherein said C₁₋₆alkyl, C₃₋₉cycloalkyl, C₂₋₆alkenyl, C₅₋₉cycloalkenyl, C₂₋₆alkynyl, C₅₋₉cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
   and wherein each R⁵ and R⁶ or R^{5a} and R^{6a} can be taken together in order to form a (4-, 5-, 6-, or 7-membered) heterocycle which can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O— alkyl, —OCF₃, —CHF₂, —OCHF₂, cyano, nitro, —C(O)OH; NH₂; —NHalkyl, and —N(alkyl)₂;
cycle A is selected from aryl; heteroaryl; cycloalkyl; and heterocycle;
   wherein said aryl, heteroaryl, cycloalkyl and heterocycle can be unsubstituted or substituted with one or more R⁷;
each R⁷ is independently selected from halogen; hydroxyl; sulfhydryl; ═O; ═S; —OZ¹; —SZ¹; —SCF₃; —SF5; —CF₃; —OCF₃; —CHF₂; —OCHF₂; —NZ³Z⁴; cyano; alkyl; cycloakyl; heteroalkyl; aryl; heteroaryl; heterocycle; arylalkyl; arylheteroalkyl; heteroarylalkyl; heteroarylheteroalkyl; heterocycle-alkyl; and heterocycle-heteroalkyl;
   wherein said alkyl, cycloakyl, heteroalkyl, aryl, heteroaryl, heterocycle, arylalkyl, arylheteroalkyl, heteroarylalkyl, heteroarylheteroalkyl, heterocycle-alkyl or heterocycle-heteroalkyl can be unsubstituted or substituted with one or more substituents selected from alkyl, cycloalkyl, hydroxyl, ═O, halogen, —SH, ═S, —CF₃, —O-alkyl, —OCF₃, —CHF₂; —OCHF₂, cyano;
each R^{a} and R⁹ are independently selected from hydrogen; halogen; hydroxyl; sulfhydryl; ═O; ═S; —OZ^{1a}; —SZ^{1a}; —SCF₃; —SF5; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; —NZ^{3a}Z^{4a}; —NZ^{3a}S(O)₂Z^{1a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; alkyl; cycloakyl; heteroalkyl; aryl; heteroaryl; heterocycle; arylalkyl; arylheteroalkyl; heteroarylalkyl; heteroarylheteroalkyl; heterocycle-alkyl; and heterocycle-heteroalkyl;
   wherein said alkyl, cycloakyl, heteroalkyl, aryl, heteroaryl, heterocycle, arylalkyl, arylheteroalkyl, heteroarylalkyl, heteroarylheteroalkyl, heterocycle-alkyl or heterocycle-heteroalkyl can be unsubstituted or substituted with one or more substituents selected from alkyl, cycloalkyl, hydroxyl, ═O, halogen, —SH, ═S, —CF₃, —O-alkyl, —OCF₃, —CHF₂; —OCHF₂, cyano, —C(O)OH, —NH₂, —NHalkyl, and —N(alkyl)₂;
each of R^{8a} and R^{9a}, are independently selected from hydrogen; hydroxyl; sulfhydryl; —OZ^{1a}; —SZ^{1a}; —SCF₃; —SF5; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z⁴a: —CF₃; —OCF₃; —CHF₂: —OCHF₂: —NZ^{3a}Z⁴a: —NZ^{3a}S(O)₂Z^{2a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; -alkyl; cycloakyl; heteroalkyl; aryl; heteroaryl; heterocycle; arylalkyl; arylheteroalkyl; heteroarylalkyl; heteroarylheteroalkyl; heterocycle-alkyl; and heterocycle-heteroalkyl;
   wherein said alkyl, cycloakyl, heteroalkyl, aryl, heteroaryl, heterocycle, arylalkyl, arylheteroalkyl, heteroarylalkyl, heteroarylheteroalkyl, heterocycle-alkyl or heterocycle-heteroalkyl can be unsubstituted or substituted with one or more substituents selected from alkyl, cycloalkyl, hydroxyl, ═O, halogen, —SH, ═S, —CF₃, —O-alkyl, —OCF₃, —CHF₂; —OCHF₂, cyano, —C(O)OH, —NH₂, —NHalkyl, and —N(alkyl)₂;
each Z¹ and Z^{1a} is independently selected from alkyl; alkenyl; cycloalkenyl; alkynyl; cycloalkyl; cycloalkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; or heterocycle-heteroalkynyl;
   wherein said alkyl, cycloalkyl, alkenyl, alkynyl, cycloalkyl, cycloalkenyl, cycloalkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, —C(O)OH; NH₂; —NHalkyl, and —N(alkyl)₂;
each Z² and Z^{2a} is independently selected from hydroxyl; alkyl; cycloalkyl; alkenyl; cycloalkenyl; alkynyl; cycloalkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; or heterocycle-heteroalkynyl;
   wherein said alkyl, cycloalkyl, alkenyl, cycloalkenyl, alkynyl, cycloalkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, —C(O)OH; NH₂; —NHalkyl, and —N(alkyl)₂;
each Z³, Z^{3a}, Z⁴, and Z^{4a} is independently selected from hydrogen; alkyl; cycloalkyl; alkenyl; cycloalkenyl; alkynyl; cycloalkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; or heterocycle-heteroalkynyl;
   wherein said alkyl, cycloalkyl, alkenyl, cycloalkenyl, alkynyl, cycloalkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂; and wherein each Z³ and Z⁴ or Z^{3a} and Z^{4a} can be taken together in order to form a (4-, 5-, 6-, or 7-membered) heterocycle which can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂.

In a specific embodiment of the present disclosure, the compounds have a structure according to formulas (II), (IIa), (IIb), (IIc), (IId), (IIe), and (IIf) or an isomer (preferably a stereo-isomer or a tautomer), a solvate, a salt (preferably a pharmaceutically acceptable salt) or a prodrug thereof, whereby the different substituents n, R¹, R², R³, R^{3a}, R⁴, R^{4a}, R⁵, R^{5a}, R^{5b}, R⁶, R^{6e}, R^{6b}, cycle A, R⁷, R⁸, R⁹, R^{8a}, R^{9a}, Z¹, Z^{1a} Z², Z^{2a}, Z³, Z^{3a}, Z⁴, and Z^{4a} have the same meaning as defined herein in the different statements, aspects or embodiments for formula (I), more specifically as in statement 1, 2, 3, 4, 5, 6, 7, or 8 herein.

11. A compound of formulas (III), (IIIa), (IIIb), (IIIc), (IIId), (IIIe), (IIIf) and (IIIg) or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof,

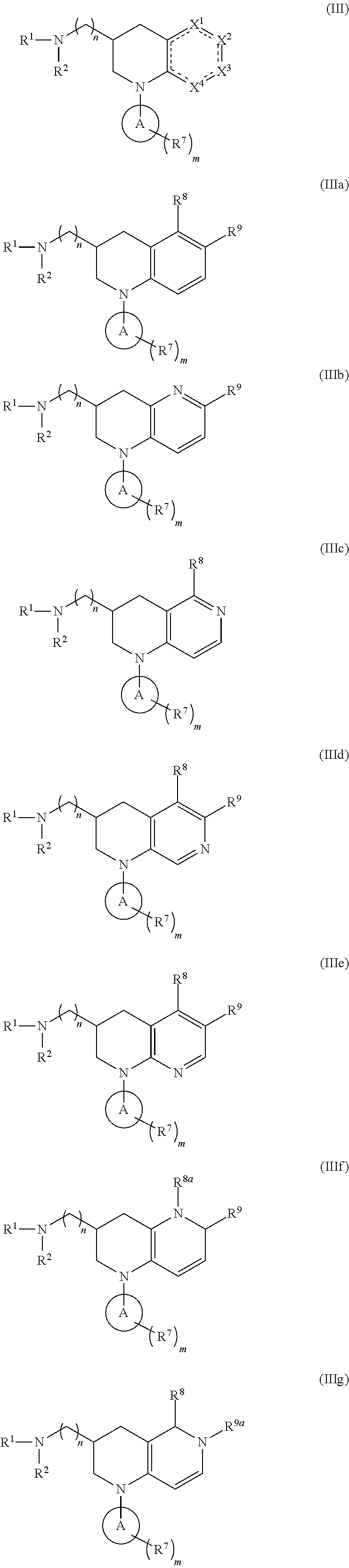

wherein:
each of n, R¹, R², cycle A, R⁷, X¹, X², X³, X⁴, R⁸, R⁹, R^{5a} and R^{9a} have the same meaning as defined herein in the different subgroups, statements, aspects or embodiments; more in particular as in the statements 1, 2, 3, 4, 5, 6, 7, 8, and 9 herein;
m is selected from 0; 1; 2, 3 and 4.

12. A compound of formulas (IV), (IVa), (IVb), (IVc), (IVd), (IVe), (IVf) and (IVg) or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof,

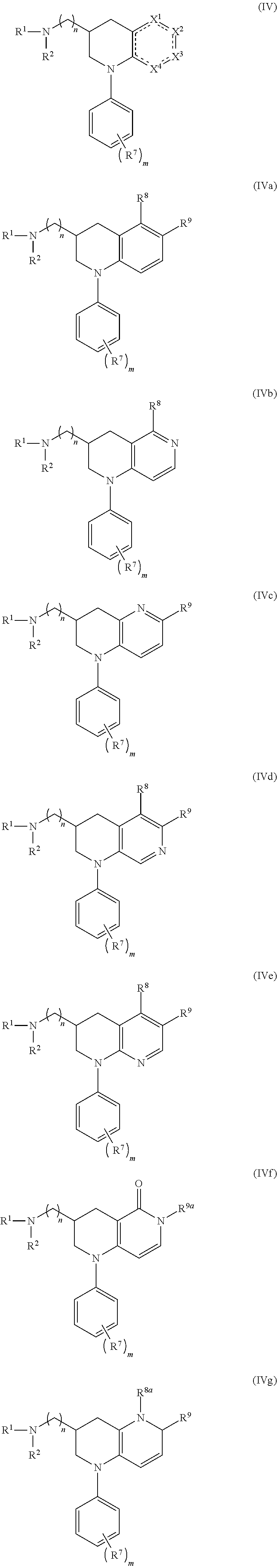

wherein:
each of n, R¹, R², cycle A, R⁷, X¹, X², X³, X⁴, R⁸, R⁹, R^{8a} and R^{9a} have the same meaning as defined herein in the different subgroups, statements, aspects or embodiments; more in particular as in the statements 1, 2, 3, 4, 5, 6, 7, 8, and 9 herein;
m is selected from 0; 1; 2, 3 and 4.

13. A compound of formulas (V), (Va), (Vb), (Vc), (Vd) and (Ve) or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof,

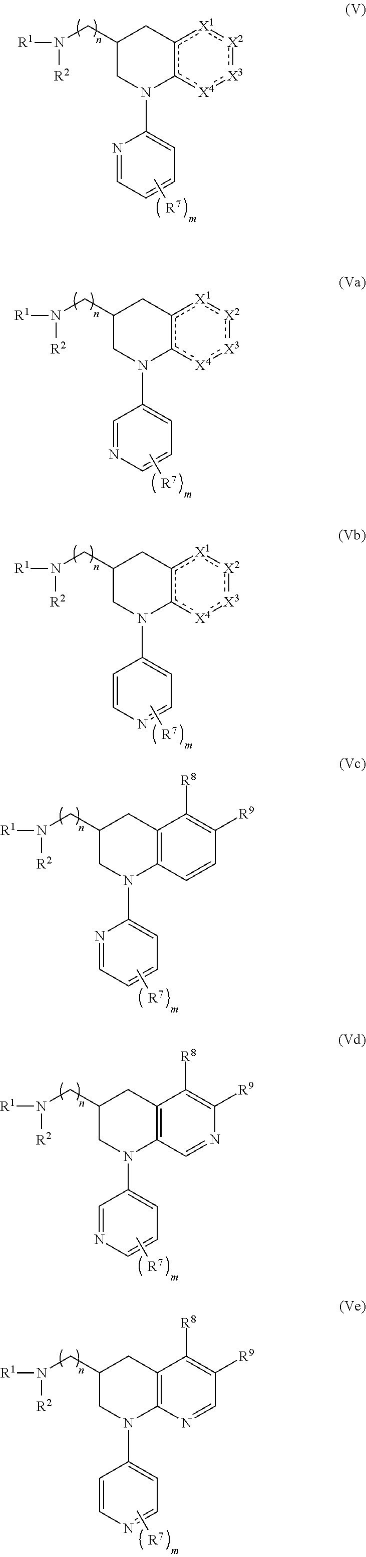

wherein:
each of n, R¹, R², cycle A, R⁷, X¹, X², X³, X⁴, R⁸, R⁹, R^{8a} and R^{9a} have the same meaning as defined herein in the different subgroups, statements, aspects or embodiments; more in particular as in the statements 1, 2, 3, 4, 5, 6, 7, 8, and 9 herein;
m is selected from 0; 1; 2, 3 and 4.

14. A compound of formula (VI) and (VIa) or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof,

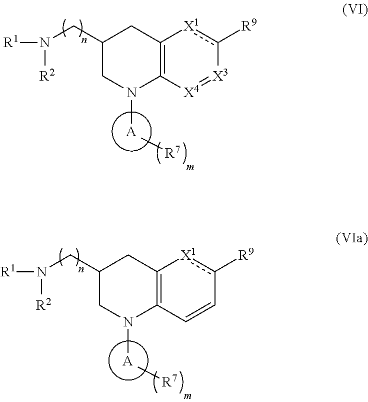

wherein:
n is selected from 0; 1; and 2;
m is selected from 1; 2; and 3;
the dotted line—represents an optional double bond;
R¹ is selected from —C(O)R³; —C(O)OR⁴; —C(O)NR⁵R⁶; —S(O)₂R^{3a}; —S(O)R^{4a}; —S(O)₂NR^{5a}R^{6a}; —S(O)(NR^{5a})R^{4a}; —S(NR^{5a})(NR^{6a})R^{3a}; and —P(O)R^{5b}R^{6b};
R² is selected from hydrogen; C₁₋₆alkyl; C₃₋₉cycloalkyl; and C₁₋₆heteroalkyl;
each R³ and R^{3a} is independently selected from hydroxyl; C₁₋₆alkyl; C₃₋₉cycloakyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; C₂₋₆ heteroalkynyl;
   wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from C₁₋₆alkyl, C₃₋₉ cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂;
each R⁴ and R^{4a} is independently selected from C₁₋₆alkyl; C₃₋₉cycloakyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; C₂₋₆ heteroalkynyl;
   wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each R⁵, R^{5a}, R^{5b}, R⁶, R^{6a} and R^{5b} is independently selected from hydrogen; C₁₋₆alkyl; C₃₋₉cycloakyl; C₂₋₆alkenyl; C₅₋₉cycloalkenyl; C₂₋₆alkynyl; C₅₋₉cycloalkynyl; C₁₋₆ heteroalkyl; C₂₋₆heteroalkenyl; and C₂₋₆heteroalkynyl;
   wherein said C₁₋₆alkyl, C₃₋₉cycloakyl, C₂₋₆alkenyl, C₅₋₉cycloalkenyl, C₂₋₆alkynyl, C₅₋₉cycloalkynyl, C₁₋₆heteroalkyl, C₂₋₆heteroalkenyl and C₂₋₆heteroalkynyl, can be unsubstituted or substituted with one or more substituents selected from C₁₋₆ alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆ alkyl, and —N(C₁₋₆alkyl)₂;
cycle A is selected from aryl; heteroaryl; C₃₋₉cycloalkyl; and heterocycle;
each R⁷ is independently selected from halogen; hydroxyl; sulfhydryl; ═O; ═S; —OZ¹; —SZ¹; —SCF₃; —SF₅; —CF₃; —OCF₃; —CHF₂; —OCHF₂; —NZ³Z⁴; cyano; C₁₋₆alkyl; C₃₋₉cycloakyl; C₁₋₆ heteroalkyl; aryl; heteroaryl; heterocycle; arylC₁₋₆alkyl; arylC₁₋₆heteroalkyl; heteroarylC₁₋₆alkyl; heteroarylC₁₋₆heteroalkyl; heterocycle-C₁₋₆alkyl; and heterocycle-C₁₋₆heteroalkyl;
   wherein said C₁₋₆alkyl, C₃₋₉cycloakyl, C₁₋₆heteroalkyl, aryl, heteroaryl, heterocycle, arylC₁₋₆alkyl, arylC₁₋₆heteroalkyl, heteroarylC₁₋₆alkyl, heteroarylC₁₋₆heteroalkyl, heterocycle-C₁₋₆alkyl, and heterocycle-C₁₋₆heteroalkyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, —C(O)OH, —NH₂, —NHC₁₋₆alkyl, and —N(C₁₋₆alkyl)₂;
X¹ is selected from CR⁸; N; and NR^{8a}; whereby X¹ can be NR^{8a} when R⁹ is C═O or C═S and the dotted line—is not a double bond;
X³ is selected from CH; and N;
X⁴ is selected from CH; and N;
each R⁸ and R⁹ are independently selected from hydrogen; halogen; hydroxyl; sulfhydryl; ═O; ═S; —OZ^{1a}; —SZ^{1a}; —SCF₃; —SF₅; —CF₃; —OCF₃; —CHF₂; —OCHF₂; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —NZ^{3a}Z^{4a}; —NZ^{3a}S(O)₂Z^{1a}; —NZ^{3a}C(O)Z^{1a} —NZ^{3a}C(O)NZ^{3a}Z^{4a}; cyano; —C(O)Z^{2a}; —C(O)OZ^{1a}; —C(O)NZ^{3a}Z^{4a}; —P(O)Z^{3a}Z^{4a}; C₁₋₆alkyl; C₃₋₉cycloakyl; C₁₋₆heteroalkyl; aryl; heteroaryl; heterocycle; arylC₁₋₆alkyl; arylC₁₋₆heteroalkyl; heteroarylC₁₋₆alkyl; heteroarylC₁₋₆heteroalkyl; heterocycle-C₁₋₆alkyl; and heterocycle-C₁₋₆ heteroalkyl;
   wherein said C₁₋₆alkyl, C₃₋₉cycloakyl, C₁₋₆heteroalkyl, aryl, heteroaryl, heterocycle, arylC₁₋₆ alkyl, arylC₁₋₆heteroalkyl, heteroarylC₁₋₆alkyl, heteroarylC₁₋₆heteroalkyl, heterocycle-C₁₋₆ alkyl and heterocycle-C₁₋₆heteroalkyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
R^{8a} is selected from hydrogen; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₁₋₆heteroalkyl; C₁₋₆alkyl; C₃₋₉cycloakyl; C₁₋₆heteroalkyl; aryl; heteroaryl; heterocycle; arylC₁₋₆alkyl; arylC₁₋₆heteroalkyl; heteroarylC₁₋₆alkyl; heteroarylC₁₋₆heteroalkyl; heterocycle-C₁₋₆alkyl; and heterocycle-C₁₋₆heteroalkyl;
   wherein said C₁₋₆alkyl, C₃₋₉cycloakyl, C₁₋₆heteroalkyl, aryl, heteroaryl, heterocycle, arylC₁₋₆ alkyl, arylC₁₋₆heteroalkyl, heteroarylC₁₋₆alkyl, heteroarylC₁₋₆heteroalkyl, heterocycle-C₁₋₆ alkyl and heterocycle-C₁₋₆heteroalkyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each Z¹ and Z^{1a} is independently selected from C₁₋₆alkyl; C₃₋₉cycloakyl; C₁₋₆heteroalkyl; aryl; heteroaryl; heterocycle; arylC₁₋₆alkyl; arylC₁₋₆heteroalkyl; heteroarylC₁₋₆alkyl; heteroarylC₁₋₆ heteroalkyl; heterocycle-C₁₋₆alkyl; and heterocycle-C₁₋₆heteroalkyl;
   wherein said C₁₋₆alkyl, C₃₋₉cycloakyl, C₁₋₆heteroalkyl, aryl, heteroaryl, heterocycle, arylC₁₋₆alkyl, arylC₁₋₆heteroalkyl, heteroarylC₁₋₆alkyl, heteroarylC₁₋₆heteroalkyl, heterocycle-C₁₋₆alkyl and heterocycle-C₁₋₆heteroalkyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each Z² and Z^{2a} is independently selected from hydroxyl; C₁₋₆alkyl; C₃₋₉cycloakyl; C₁₋₆ heteroalkyl; aryl; heteroaryl; heterocycle; arylC₁₋₆alkyl; arylC₁₋₆heteroalkyl; heteroarylC₁₋₆alkyl; heteroarylC₁₋₆heteroalkyl; heterocycle-C₁₋₆alkyl; and heterocycle-C₁₋₆heteroalkyl;
   wherein said C₁₋₆alkyl, C₃₋₉cycloakyl, C₁₋₆heteroalkyl, aryl, heteroaryl, heterocycle, arylC₁₋₆ alkyl, arylC₁₋₆heteroalkyl, heteroarylC₁₋₆alkyl, heteroarylC₁₋₆heteroalkyl, heterocycle-C₁₋₆ alkyl and heterocycle-C₁₋₆heteroalkyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂;
each Z³, Z^{3a}, Z⁴, and Z^{4a} is independently selected from hydrogen; C₁₋₆alkyl; C₃₋₉cycloakyl; C₁₋₆ heteroalkyl; aryl; heteroaryl; heterocycle; arylC₁₋₆alkyl; arylC₁₋₆heteroalkyl; heteroarylC₁₋₆alkyl; heteroarylC₁₋₆heteroalkyl; heterocycle-C₁₋₆alkyl; and heterocycle-C₁₋₆heteroalkyl;
   wherein said C₁₋₆alkyl, C₃₋₉cycloakyl, C₁₋₆heteroalkyl, aryl, heteroaryl, heterocycle, arylC₁₋₆alkyl, arylC₁₋₆heteroalkyl, heteroarylC₁₋₆alkyl, heteroarylC₁₋₆ heteroalkyl, heterocycle-C₁₋₆alkyl and heterocycle-C₁₋₆heteroalkyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂;
   and wherein each Z³ and Z⁴ or Z^{3a} and Z^{4a} can be taken together in order to form a (4-, 5-, 6-, or 7-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂;

15. A compound of formula (VII), (VIIa) and (VIIb) or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof,

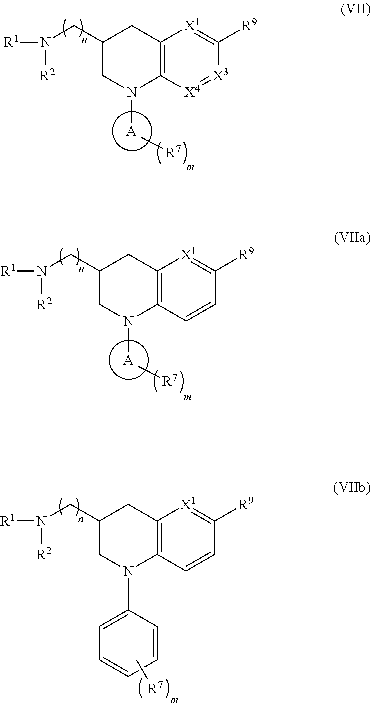

wherein:
each of n, m, R², cycle A, R⁷, X¹, X³, X⁴ and R⁹ have the same meaning as defined herein in the different subgroups, statements, aspects or embodiments; more in particular as in the statements 1, 2, 3, 4, 5, 6, 7, 8, 9, 10, 11, 12, and 13 herein;
R¹ is selected from —C(O)R³; and —S(O)₂R^{3a}.

16. A compound of formula (VIII), (VIIIa), (VIIIb) and (VIIIc) or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof,

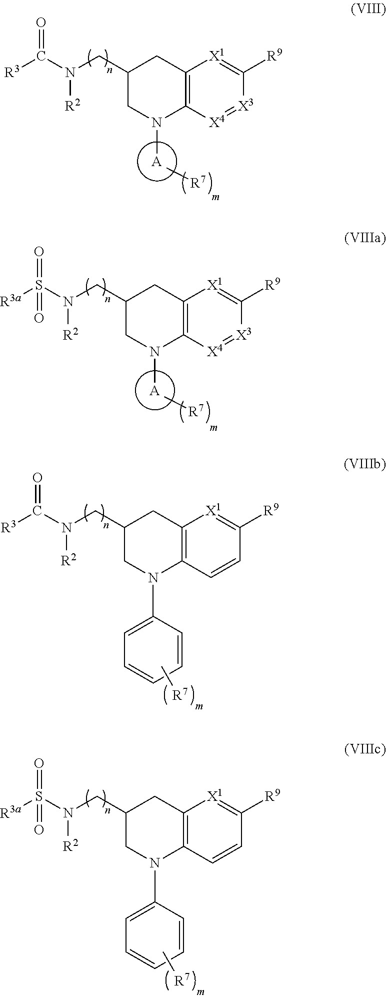

wherein:
n is selected from 0; and 1;
m is selected from 1; 2; and 3;
R² is selected from hydrogen; and C₁₋₆alkyl;
each R³ and R^{3a} is independently selected from C₁₋₆alkyl; C₂₋₆alkenyl; and C₂₋₆alkynyl;
   wherein said C₁₋₆alkyl, C₂₋₆alkenyl and C₂₋₆alkynyl can be unsubstituted or substituted with one or more substituents selected from C₁₋₆alkyl, C₃₋₉cycloalkyl, C₂₋₆alkenyl, C₂₋₆ alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂;
cycle A is selected from aryl; and heteroaryl;
each R⁷ is independently selected from halogen; hydroxyl; sulfhydryl; ═O; ═S; —OZ¹; —SZ¹; —SCF₃; —SF₅; —CF₃; —OCF₃; —CHF₂; —OCHF₂; cyano; C₁₋₆alkyl; and C₁₋₆heteroalkyl;
   wherein said C₁₋₆alkyl and C₁₋₆heteroalkyl can be unsubstituted or substituted with one or more substituents selected from C₁₋₆alkyl, C₃₋₉cycloalkyl, C₂₋₆alkenyl, C₂₋₆ alkynyl, hydroxyl, ═O, halogen, —SH, ═S, —CF₃, —O—C₁₋₆alkyl, —OCF₃, —CHF₂, —OCHF₂, cyano, —C(O)OH, —NH₂, —NHC₁₋₆alkyl, and —N(C₁₋₆alkyl)₂;
X¹ is selected from CR⁸; and N;
X³ is selected from CH; and N;
X⁴ is selected from CH; and N;
R⁸ is selected from hydrogen; halogen; hydroxyl; sulfhydryl; —OZ^{1a}; —CF₃; —OCF₃; —CHF₂; —OCHF₂; —NZ^{3a}Z^{4a}; —NZ^{3a}S(O)₂Z^{1a}; —NZ^{3a}C(O)Z^{1a}; cyano; —C(O)OZ^{1a}; —C(O)NZ^{3a}Z^{4a}; C₁₋₆alkyl; C₁₋₆heteroalkyl; aryl; heteroaryl; heterocycle; arylC₁₋₆alkyl; heteroarylC₁₋₆alkyl; and heterocycle-C₁₋₆alkyl;
   wherein said C₁₋₆alkyl, C₁₋₆heteroalkyl, aryl, heteroaryl, heterocycle, arylC₁₋₆alkyl, heteroarylC₁₋₆alkyl or heterocycle-C₁₋₆alkyl can be unsubstituted or substituted with one or more substituents selected from alkyl, hydroxyl, halogen, —CF₃, —O—C₁₋₆alkyl, —OCF₃, —CHF₂; —OCHF₂, cyano, —NH₂;
R⁹ is selected from hydrogen; halogen; hydroxyl; sulfhydryl; ═O; ═S; —OZ^{1a}; —CF₃; —OCF₃; —CHF₂; —OCHF₂; —NZ^{3a}Z^{4a}; C₁₋₆alkyl; and C₁₋₆heteroalkyl;
wherein said C₁₋₆alkyl and C₁₋₆heteroalkyl can be unsubstituted or substituted with one or more substituents selected from hydroxyl, halogen, —CF₃, —O—C₁₋₆alkyl, —OCF₃, —CHF₂; —OCHF₂, cyano, —NH₂;-each Z¹ and Z^{1a} is independently selected from C₁₋₆alkyl; and C₁₋₆heteroalkyl;
   wherein said C₁₋₆alkyl and C₁₋₆heteroalkyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each Z^{3a} and Z^{4a} is independently selected from hydrogen; C₁₋₆alkyl; C₃₋₉cycloakyl; C₁₋₆heteroalkyl; aryl; heteroaryl; and heterocycle;
   wherein said C₁₋₆alkyl, C₃₋₉cycloakyl, C₁₋₆heteroalkyl, aryl, heteroaryl, and heterocycle can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂.

17. The compound according to statements 1 to 16, wherein each R³ and R^{3a} is independently selected from C₂₋₆alkenyl; and C₂₋₆alkynyl;
wherein said C₂₋₆alkenyl and C₂₋₆alkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂.

18. The compound according to statements 1 to 17, wherein n is 1.

19. The compound according to statements 1 to 17, wherein n is 0.

20. The compound according to statements 1 to 19, wherein R^{a} is selected from hydrogen; halogen; —OZ^{1a}; —CF₃; —OCF₃; —CHF₂; —OCHF₂; —NZ^{3a}Z^{4a}; cyano; C₁₋₆alkyl; C₁₋₆heteroalkyl; arylC₁₋₆alkyl; heteroarylC₁₋₆alkyl; and heterocycle-C₁₋₆alkyl;
wherein said C₁₋₆alkyl, C₁₋₆heteroalkyl, arylC₁₋₆alkyl, heteroarylC₁₋₆alkyl or heterocycle-C₁₋₆ alkyl can be unsubstituted or substituted with one or more substituents selected from alkyl, hydroxyl, halogen, —CF₃, —O—C₁₋₆alkyl, —OCF₃, —CHF₂; —OCHF₂, cyano, —NH₂;

21. The compound according to statements 1 to 19, wherein R^{a} is selected from halogen; —OZ^{1a}; —CF₃; —OCF₃; —CHF₂; —OCHF₂; —NZ^{3a}Z^{4a}; cyano; C₁₋₆alkyl; C₁₋₆heteroalkyl; arylC₁₋₆alkyl; heteroarylC₁₋₆alkyl; and heterocycle-C₁₋₆alkyl;
wherein said C₁₋₆alkyl, C₁₋₆heteroalkyl, arylC₁₋₆alkyl, heteroarylC₁₋₆alkyl or heterocycle-C₁₋₆ alkyl can be unsubstituted or substituted with one or more substituents selected from alkyl, hydroxyl, halogen, —CF₃, —O—C₁₋₆alkyl, —OCF₃, —CHF₂; —OCHF₂, cyano, —NH₂

22. The compound according to statements 1 to 21, wherein R⁹ is selected from hydrogen; halogen; —OZ^{1a}; —CF₃; —OCF₃; —CHF₂; —OCHF₂; —NZ^{3a}Z^{4a}; C₁₋₆alkyl; and C₁₋₆heteroalkyl;
wherein said C₁₋₆alkyl and C₁₋₆heteroalkyl can be unsubstituted or substituted with one or more substituents selected from hydroxyl, halogen, —CF₃, —O—C-alkyl, —OCF₃, —CHF₂; —OCHF₂, cyano, —NH₂;

23. A compound of formula (I) according to statement 5 herein or any other statement, embodiments or aspects thereof, or according to formulas (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb) and (VIIIc) or any other formulas, statements, embodiments or aspects thereof
wherein:
n is selected from 0; 1; and 2;
if n is 1 or 2, then
   R¹ is selected from C₁₋₆alkyl; C₃₋₉cycloalkyl; C₂₋₆alkenyl; C₅₋₉cycloalkenyl; C₂₋₆alkynyl; C₅₋₉ cycloalkynyl; C₁₋₆heteroalkyl; C₂₋₆heteroalkenyl; C₂₋₆heteroalkynyl; —C(O)H; —C(O)R³; —C(O)OR⁴; —C(O)NR⁵R⁶; —S(O)₂R^{3a}; —S(O)R^{4a}; —S(O)₂NR^{5a}R^{6a}; —S(O)(NR^{5a})R^{4a}; —S(NR^{5a})(NR^{6a})R^{3a}; and —P(O)R^{5b}R^{6b};
      wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆alkyl)₂, —S(O)(NH)C₁₋₆alkyl, —S(O)(NC1-6alkyl)C₁₋₆alkyl, —S(NH)(NH)C₁₋₆alkyl, —NH₂, —NHC₁₋₆alkyl, —N(C₁₋₆alkyl)₂; and
   each R³ and R^{3a} is independently selected from hydroxyl; C₂₋₆alkyl; C₃₋₉cycloalkyl; C₂₋₆ alkeny; C₅₋₉cycloalkenyl; C₂₋₆alkynyl; C₅₋₉cycloalkynyl; C₁₋₆heteroalkyl; C₂₋₆ heteroalkenyl; and C₂₋₆heteroalkynyl;
      wherein said C₂₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
if n is 0, then
   R¹ is selected from C₂₋₄alkyl; C₆akyl; C₃₋₉cycloalkyl; C₂₋₆alkenyl; C₅₋₉cycloalkenyl; C₂₋₆ alkynyl; C₅₋₉cycloalkynyl; C₁₋₆heteroalkyl; C₂₋₆heteroalkenyl; C₂₋₆heteroalkynyl; —C(O)H; —C(O)R³; —C(O)OR⁴; —C(O)NR⁵R⁶; —S(O)₂R^{3a}; —S(O)R^{4a}; —S(O)₂NR^{5a}R^{6a}; —S(O)(NR^{5a})R^{4a}; —S(NR^{5a})(NR^{6a})R^{3a}; and —P(O)R^{5b}R^{6b};
      wherein said C₂-4akyl; C₆alky; C₃₋₉cycloalkyl, C₂₋₆alkenyl, C₅₋₉cycloalkenyl, C₂₋₆ alkynyl, C₅₋₉cycloalkynyl, C₁₋₆heteroalkyl, C₂₋₆heteroalkenyl and C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆alkyl)₂, —S(O)(NH)C₁₋₆alkyl, —S(O)(NC1-6alkyl)C₁₋₆alkyl, —S(NH)(NH)C₁₋₆alkyl, —NH₂, —NHC₁₋₆alkyl, —N(C₁₋₆alkyl)₂; and
   each R³ and R^{3a} is independently selected from hydroxyl; C₃₋₆alkyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; and C₂₋₆ heteroalkynyl;
      wherein said C₃₋₆alkyl, C₂₋₆alkenyl, C₅₋₉cycloalkenyl, C₂₋₆alkynyl, C₅₋₉cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
   each R⁴ and R^{4a} is independently selected from C₁₋₃alkyl; C₅₋₆alkyl; C₃₋₉cycloalkyl; C₂₋₆ alkenyl; C₅₋₉cycloalkenyl; C₂₋₆alkynyl; C₅₋₉cycloalkynyl; C₁₋₆heteroalkyl; C₂₋₆ heteroalkenyl; and C₂₋₆heteroalkynyl;
      wherein said C₁₋₃alkyl; C₅₋₆alkyl; C₃₋₉cycloalkyl, C₂₋₆alkenyl, C₅₋₉cycloalkenyl, C₂₋₆ alkynyl, C₅₋₉cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
if n is 0, 1 or 2, R¹ and R² can also be taken together to form a (3-; 4-; 5-; 6- or 7-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆ alkyl, —CONH₂, —CONHC₁₋₆alkyl, —CON(C₁₋₆alkyl)₂, —SO₂C₁₋₆alkyl, —SO₂NH₂, —SO₂NHC₁₋₆ alkyl, —SO₂N(C₁₋₆alkyl)₂, —S(O)(NH)C₁₋₆alkyl, —S(O)(NC₁₋₆alkyl)C₁₋₆alkyl, —S(NH)(NH)C₁₋₆ alkyl, —NH₂ —NHC₁₋₆alkyl, —N(C₁₋₆alkyl)₂;
provided that R¹ is not methyl; or in a particular embodiment provided that cycle A is substituted with at least one R⁷; or in another particular embodiment provided that one of R⁸ or R⁹ is not hydrogen.

24. A compound of formula (I) according to statement 5 herein or any other statement, embodiments or aspects thereof, or according to formulas (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb) and (VIIIc) or any other formulas, statements, embodiments or aspects thereof
wherein
n is selected from 0; 1; and 2;
R¹ is selected from —C(O)R³; a n d —S(O)₂R^{3a};
each R³ and R^{3a} is independently selected from hydroxyl; C₂₋₆alkenyl; C₅₋₉cycloalkenyl; C₂₋₆ alkynyl; C₅₋₉cycloalkynyl;
   wherein said C₂₋₆alkenyl, C₅₋₉cycloalkenyl, C₂₋₆alkynyl, C₅₋₉cycloalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂.
      25. A compounds of formula (I), according to statement 5, herein or any other statement, embodiments or aspects thereof, or according to formulas (II), (la), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb) and (VIIIc) or any other formulas, statements, embodiments or aspects thereof
wherein:
R⁸ is independently selected from hydrogen; halogen; hydroxyl; sulfhydryl; ═O; ═S; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{1a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂-heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
R⁹ is independently selected from hydrogen; halogen; hydroxyl; sulfhydryl; ═O; ═S; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{1a}; —NZ^{3a}C(O)Z^{1a}; —NZ^{3a}C(O)NZ^{3a}Z^{4a}; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂; and each Z^{1a} is independently selected from C₂₋₆alkyl; C₃₋₉cycloakyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; C₂₋₆ heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆ alkenyl; heterocycle-C₂₋₆alkynyl; heterocycle-C₁₋₆heteroalkyl; heterocycle-C₂₋₆ heteroalkenyl; and heterocycle-C₂₋₆heteroalkynyl;
      wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆ heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;

26. A compounds of formula (I), according to statement 5 herein or any other statement, embodiments or aspects thereof, or according to formulas (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb) and (VIIIc) or any other formulas, statements, embodiments or aspects thereof wherein:
R⁸ is selected from halogen; hydroxyl; sulfhydryl; ═O; ═S; —OZ^{1a}; —SZ^{1a}; —SCF₃; —SF5; —S(O)Z^{1a}; —S(O)(NZ^{3a})Z^{1a}; —S(NZ^{3a})(NZ^{3a})Z^{1a}; —S(O)₂Z^{2a}, —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{1a}; —NZ^{3a}C(O)Z^{1a}; —NZ^{3a}C(O)NZ^{3a}Z⁴a cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆ heteroalkenyl; arylC₂₋₆heteroalkynyl; heteroarylC₁₋₆alkyl; heteroarylC₂₋₆alkenyl; heteroarylC₂₋₆ alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆ heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
R⁹ is independently selected from hydrogen; halogen; hydroxyl; sulfhydryl; ═O; ═S; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{1a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂.

27. A compound of formula (I), according to statement 5 herein or any other statement, embodiments or aspects thereof, or according to formulas (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb) and (VIIIc) or any other formulas, statements, embodiments or aspects thereof
wherein:
cycle A is selected from aryl; heteroaryl; C₃₋₉cycloalkyl; and heterocycle;
   wherein said aryl, heteroaryl, C₃₋₉cycloalkyl and heterocycle is substituted with one or more R⁷; more in particular with 1, 2, 3 or 4 R⁷; yet more in particular with 1, 2 or 3 R⁷; still more in particular with 1 or 2 R⁷; still more in particular with 2, 3 or 4 R⁷; yet still more in particular with at least 1 R⁷;
when cycle A is phenyl, then each R⁷ is independently selected from F; Br; I; hydroxyl; sulfhydryl; ortho-OMe; para-OMe; ortho-OEt; m-OEt; —OZ¹; —SZ¹; —SCF3; —SF₅; —CF₃; —OCF₃; —CHF₂; —OCHF₂; —NZ³Z⁴; —NZ³C(O)Z¹; cyano; —C(O)Z²; —C(O)OZ¹; —C(O)NZ³Z⁴; C₁₋₆alkyl; C₃₋₆ cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylalkynyl; arylC₁₋₆heteroalkyl; arylC₂₋₆ heteroalkenyl; arylC₂₋₆heteroalkynyl; heteroarylC₁₋₆alkyl; heteroarylC₂₋₆alkenyl; heteroarylC₂₋₆ alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆ heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆ heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆ alkyl, —OCF₃, —CHF₂; —OCHF₂, cyano, nitro, —C(O)OH, —NH₂, —NHC₁₋₆alkyl, and —N(C₁₋₆ alkyl)₂; and
   wherein each Z¹ is independently selected from C₃₋₉alkyl; C₃₋₉cycloakyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; C₂₋₆ heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆ alkenyl; heterocycle-C₂₋₆alkynyl; heterocycle-C₁₋₆heteroalkyl; heterocycle-C₂₋₆ heteroalkenyl; and heterocycle-C₂₋₆heteroalkynyl;
      wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆ heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
when cycle A is heteroaryl, then each R⁷ is independently selected from halogen; hydroxyl; sulfhydryl; ═O; ═S; —OZ¹; —SZ¹; —SCF₃; —SF₅; —CF₃; —OCF₃; —CHF₂; —OCHF₂; —NZ³Z⁴; —NZ³C(O)Z¹; cyano; —C(O)Z²; —C(O)OZ¹; —C(O)NZ³Z⁴; C₂₋₆alkyl; C₂₋₆alkenyl; C₂₋₆alkynyl; C₂-₃heteroalkyl; C₅₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylalkynyl; arylC₁₋₆heteroalkyl; arylC₂₋₆heteroalkenyl; arylC₂₋₆ heteroalkynyl; heteroarylC₁₋₆alkyl; heteroarylC₂₋₆alkenyl; heteroarylC₂₋₆alkynyl; heteroarylC₁₋₆ heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆ heteroalkenyl; and heterocycle-C₂₋₆heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆ heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆ alkyl, —OCF₃, —CHF₂; —OCHF₂, cyano, nitro, —C(O)OH, —NH₂, —NHC₁₋₆alkyl, and —N(C₁₋₆ alkyl)₂, and
   wherein each Z¹ is independently selected from C₂₋₃alkyl; C₅₋₆alkyl; C₃₋₉cycloakyl; C₂₋₆ alkenyl; C₅₋₉cycloalkenyl; C₂₋₆alkynyl; C₅₋₉cycloalkynyl; C₁₋₆heteroalkyl; C₂₋₆ heteroalkenyl; O₂₋₆heteroalkynyl; aryl; heteroaryl; heterocycle; arylC₁₋₆alkyl; arylC₂₋₆ alkenyl; arylC₂₋₆alkynyl; arylC₁₋₈heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆ heteroalkyl; heteroarylC₂₋₆heteroalkenyl; heteroarylC₂₋₆heteroalkynyl; heterocycle-C₁₋₆ alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
      wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆ heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
when cycle A is different from phenyl or heteroaryl, then each R⁷ is independently selected from halogen; hydroxyl; sulfhydryl; ═O; ═S; —OZ¹; —SZ¹; —SCF₃; —SF₅; —CF₃; —OCF₃; —CHF₂; —OCHF₂; —NZ³Z⁴; —NZ³C(O)Z¹; cyano; —C(O)Z²; —C(O)OZ¹; —C(O)NZ³Z⁴; C₁₋₆alkyl; C₃₋₆ cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylalkynyl; arylC₁₋₆heteroalkyl; arylC₂₋₆ heteroalkenyl; arylC₂₋₆heteroalkynyl; heteroarylC₁₋₆alkyl; heteroarylC₂₋₆alkenyl; heteroarylC₂₋₆ alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆ heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆ heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆ alkyl, —OCF₃, —CHF₂; —OCHF₂, cyano, nitro, —C(O)OH, —NH₂, —NHC₁₋₆alkyl, and —N(C₁₋₆alkyl)₂;

28. A compounds of formula (I), according to statement 5 herein or any other statement, embodiments or aspects thereof, or according to formulas (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb) and (VIIIc) or any other formulas, statement, embodiments or aspects thereof
wherein:
cycle A is selected from aryl; heteroaryl; C₃₋₉cycloalkyl; and heterocycle;
   wherein said aryl, heteroaryl, C₃₋₉cycloalkyl and heterocycle are substituted with one or more R⁷; more in particular with 1, 2, 3 or 4 R⁷; yet more in particular with 1, 2 or 3 R⁷; still more in particular with 1 or 2 R⁷; still more in particular with 2 or 3 R⁷; yet still more in particular with at least 1 R⁷.
1 substituent selected from R³ and R⁹ is independently selected from hydrogen; halogen; hydroxyl; sulfhydryl; ═O; ═S; —OZ^{1a}; —SZ^{1a}; —SCF₃; —SF5; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{1a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆ alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆ heteroalkynyl; heteroarylC₁₋₆-akyl; heteroarylC₂₋₆alkenyl; heteroarylC₂₋₆alkynyl; heteroarylC₁₋₆ heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆ heteroalkenyl; and heterocycle-C₂₋₆heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
the other substituent selected from R^{a} and R⁹ is independently selected from halogen; hydroxyl; sulfhydryl; ═O; ═S; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{1a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆ alkynyl; C₁₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆ heteroalkynyl; heteroarylC₁₋₆-akyl; heteroarylC₂₋₆alkenyl; heteroarylC₂₋₆alkynyl; heteroarylC₁₋₆ heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆ heteroalkenyl; and heterocycle-C₂₋₆heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
or in a particular embodiment, whereby at least one of R³ and R⁹ is not hydrogen.

29. A compounds of formula (I), according to statement 5 herein or any other statement, embodiments or aspects thereof, or according to formulas (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb) and (VIIIc) or any other formulas, statement, embodiments or aspects thereof
wherein:
cycle A is selected from unsubstituted or substituted with one or more R⁷ naphthalenyl; anthracenyl; cyclopropyl; cyclobutyl; cycloheptyl; cyclooctyl; norbornyl; fenchyl; decalinyl; adamantly; triazolyl; pyrazolyl; pyrrolyl; furanyl; imidazolyl; oxazolyl; isoxazolyl; thiazolyl; isothiazolyl; oxadiazolyl; thiadiazolyl; oxatriazolyl; pyrimidyl; pyrazinyl; pyridazinyl; triazinyl; indolyl; indolizinyl; isoindolyl; benzofuranyl; benzothiophenyl; indazolyl; benzimidazolyl; benzoxazolyl; benzisoxazolyl; benzothiazolyl; benzoisothiazolyl; dihydro-benzofuranyl; thienopyridinyl; imidazopyridinyl; benzodioxolyl; quinolinyl; isoquinolinyl; cinnolinyl; quinazolinyl; and quinoxalinyl.

30. A compound of formula (IX), or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof,

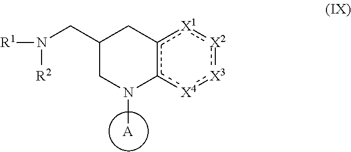

wherein:
each — represents an optional double bond, whereby maximally 3 —are a double bond at the same time;
R¹ is selected from C₁₋₆alkyl; C₃₋₉cycloalkyl; C₂₋₆alkenyl; C₅₋₉cycloalkenyl; C₂₋₆alkynyl; C₅₋₉ cycloalkynyl; C₁₋₆heteroalkyl; C₂₋₆heteroalkenyl; C₂₋₆heteroalkynyl; —C(O)H; —C(O)R³; —C(O)OR⁴; —C(O)NR⁵R⁶; —S(O)₂R^{3a}; —S(O)R^{4a}; —S(O)₂NR^{5a}R^{6a}; —S(O)(NR^{5a})R^{4a}; —S(NR^{5a})(NR^{6a})R^{3a}; and —P(O)R^{5b}R^{6b};
   wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from C₁₋₆alkyl, C₃₋₉ cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆ alkyl)₂, —S(O)(NH)C₁₋₆alkyl, —S(O)(NC₁₋₆alkyl)C₁₋₆alkyl, —S(NH)(NH)C₁₋₆alkyl, —NH₂, —NHC₁₋₆alkyl, —N(C₁₋₆alkyl)₂;
R² is selected from hydrogen; C₁₋₆alkyl; C₃₋₉cycloalkyl; and C₁₋₆heteroalkyl;
R¹ and R² can be taken together to form a (3-; 4-; 5-; 6- or 7-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)C₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆ akyl)₂, —S(O)(NH)C₁₋₆alkyl, —S(O)(NC₁₋₆alkyl)C₁₋₆alkyl, —S(NH)(NH)C₁₋₆alkyl, —NH₂, —NHC₁₋₆alkyl, —N(C₁₋₆alkyl)₂;
each R³ and R^{3a} is independently selected from hydroxyl; C₁₋₆alkyl; C₃₋₉cycloalkyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; and C₂₋₆ heteroalkynyl;
   wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁴ and R^{4a} is independently selected from C₁₋₆alkyl; C₃₋₉cycloalkyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; and C₂₋₆ heteroalkynyl;
   wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁵, R^{5a}, R^{5b}, R⁶, R^{6a} and R^{6b} is independently selected from hydrogen; C₁₋₆alkyl; C₃₋₉cycloalkyl; C₂₋₆alkenyl; C₅₋₉cycloalkenyl; C₂₋₆alkynyl; C₅₋₉cycloalkynyl; C₁₋₆heteroalkyl; C₂₋₆ heteroalkenyl; and C₂₋₆heteroalkynyl;
   wherein said C₁₋₆alkyl, C₃₋₉cycloalkyl, C₂₋₆alkenyl, C₅₋₉cycloalkenyl, C₂₋₆alkynyl, C₅₋₉cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
   and wherein each R⁵ and R⁶ or R^{5a} and R^{6a} can be taken together in order to form a (4-, 5-, 6-, or 7-membered) heterocycle which can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O— alkyl, —OCF₃, —CHF₂, —OCHF₂, cyano, nitro, —C(O)OH; NH₂; —NHalkyl, and —N(alkyl)₂;
cycle A is selected from aryl; heteroaryl; C₃₋₉cycloalkyl; and heterocycle;
   wherein said aryl, heteroaryl, C₃₋₉cycloalkyl and heterocycle can be unsubstituted or substituted with one or more R⁷;
each R⁷ is independently selected from halogen; hydroxyl; sulfhydryl; ═O; ═S; —OZ¹; —SZ¹; —SCF3; —SF₅; —CF₃; —OCF₃; —CHF₂; —OCHF₂; —NZ³Z⁴; —NZ³C(O)Z¹; cyano; —C(O)Z²; —C(O)OZ¹; —C(O)NZ³Z⁴; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylalkynyl; arylC₁₋₆ heteroalkyl; arylC₂₋₆heteroalkenyl; arylC₂₋₆heteroalkynyl; heteroarylC₁₋₆alkyl; heteroarylC₂₋₆ alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆ alkynyl; heterocycle-C₁₋₆heteroalkyl; heterocycle-C₂₋₆heteroalkenyl; and heterocycle-C₂₋₆ heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆ heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆ alkyl, —OCF₃, —CHF₂; —OCHF₂, cyano, nitro, —C(O)OH, —NH₂, —NHC₁₋₆alkyl, and —N(C₁₋₆ alkyl)₂;
X¹ is selected from CR⁸; N; and NR^{8a}; whereby X¹ can only be NR^{8a} when X² and/or X⁴ are C═O or C═S;
X² is selected from CR⁹; N; and NR^{9a}; whereby X² can only be NR^{9a} when X¹ and/or X³ are C═O or C—SH;
X³ is selected from CH; and N;
X⁴ is selected from CH; and N;
whereby maximally 2 of X¹, X², X³ and X⁴ can be a N (selected from N and NR^{8a} for X¹, from N and NR^{9a} for X², from N for X³, and from N for X⁴ respectively) at the same time;
each R⁸ and R⁹ are independently selected from hydrogen; halogen; hydroxyl; sulfhydryl; ═O; ═S; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{1a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each R^{8a} and R^{9a} are independently selected from hydrogen; hydroxyl; sulfhydryl; —OZ^{1a}; —SZ^{1a}; —SCF3; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{2a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each Z¹ and Z^{1a} is independently selected from C₁₋₆alkyl; C₃₋₉cycloakyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; C₂₋₆ heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆ alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆ alkynyl; heterocycle-C₁₋₆heteroalkyl; heterocycle-C₂₋₆heteroalkenyl; and heterocycle-C₂₋₆ heteroalkynyl;
   wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆heteroalkyl, arylC₂₋₆ heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each Z² and Z^{2a} is independently selected from hydroxyl; C₁₋₆alkyl; C₃₋₉cycloakyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; C₂₋₆ heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆heteroalkyl, arylC₂₋₆ heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂;
each Z³, Z^{3a}, Z⁴, and Z^{4a} is independently selected from hydrogen; C₁₋₆alkyl; C₃₋₉cycloakyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆ alkenyl; arylC₂₋₆alkynyl; arylC₁₋₆heteroalkyl; arylC₂₋₆heteroalkenyl; arylC₂₋₆ heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
   wherein said C₁₋₆alkyl, C₃₋₉cycloakyl, C₂₋₆alkenyl, C₅₋₉cycloalkenyl, C₂₋₆alkynyl, C₅₋₉cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆ heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆ alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆ alkyl, and —N(C₁₋₆alkyl)₂;
   and wherein each Z³ and Z⁴ or Z^{3a} and Z^{4a} can be taken together in order to form a (4-, 5-, 6-, or 7-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂;
provided that R³ is not methyl; or in a particular embodiment provided that R³ is not alkyl, more in particular is not C₁₋₆alkyl; or in a particular embodiment provided that cycle A is not phenyl; or in a particular embodiment provided that cycle A is substituted with at least one R⁷; or in another particular embodiment provided that one of R³ and R⁹ is not hydrogen.

31. The compound according to statements 1 to 30, wherein each R³ is independently selected from hydroxyl; C₂₋₆alkyl; C₃₋₉cycloalkyl; C₂₋₆alkenyl; C₅₋₉cycloalkenyl; C₂₋₆alkynyl; C₅₋₉ cycloalkynyl; C₁₋₆heteroalkyl; C₂₋₆heteroalkenyl; and C₂₋₆heteroalkynyl;
wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂.

32. The compound according to statements 1 to 31, wherein cycle A is selected from aryl; heteroaryl; C₃₋₉cycloalkyl; and heterocycle;
wherein said aryl, heteroaryl, C₃₋₉cycloalkyl and heterocycle are substituted with one or more R⁷; more in particular are substituted with 1, 2, 3 or 4 R⁷; yet more in particular are substituted with 1, 2 or 3 R⁷, still more in particular are substituted with 2, 3 or 4 R⁷.

33. The compound according to statements 1 to 31, wherein cycle A is selected from heteroaryl; C₃₋₉cycloalkyl; and heterocycle;
wherein said heteroaryl, C₃₋₉cycloalkyl and heterocycle are substituted with one or more R⁷; more in particular are substituted with 1, 2, 3 or 4 R⁷; yet more in particular are substituted with 1, 2 or 3 R⁷, still more in particular are substituted with 2, 3 or 4 R⁷.

34. The compound according to statements 1 to 31, wherein cycle A is aryl.

35. The compound according to statements 1 to 34, wherein at least one of R⁸ and R⁹ is not hydrogen.

36. The compound according to statements 1 to 35, wherein R² is selected from C₁₋₆alkyl; C₃₋₉cycloalkyl; and C₁₋₆heteroalkyl.

In a particular embodiment of the present disclosure, the compounds have a structure according to the different formulas (I), (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc) and (IX) and any other formulas described herein, more in particular according to the statements, embodiments and aspects described herein, whereby n is selected from 0; 1; and 2. More in particular n is selected from 1 and 2. Yet more in particular n is 1. Still more in particular n is 0. Yet still more in particular n is 2.

In a particular embodiment of the present disclosure, the compounds have a structure according to the different formulas (I), (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc) and (IX) and any other formulas described herein, more in particular according to the statements, embodiments and aspects described herein, whereby each — represents an optional double bond, whereby maximally 3 —are a double bond at the same time; more in particular whereby maximally 2— are a double bond at the same time; yet more in particular whereby maximally 1— is a double bond at the same time; still more in particular whereby 1, 2 or 3 —are a double bond; yet more in particular whereby 2 or 3 —are a double bond; yet more in particular whereby 3 —are a double bond; yet more in particular whereby 2 —are a double bond. When reference is made to maximally 3 or 2 —are a double bond at the same time, then this refers to maximally 3 or 2 —which are double bonds and such double bonds are non-consecutive.

In a particular embodiment of the present disclosure, the compounds have a structure according to the different formulas (I), (II), (la), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc) and (IX) and any other formulas described herein, more in particular according to the statements, embodiments and aspects described herein, whereby R¹ is selected from C₁₋₆alkyl; C₃₋₉cycloalkyl; C₂₋₆alkenyl; C₅₋₉ cycloalkenyl; C₁₋₆heteroalkyl; C₂₋₆heteroalkenyl; —C(O)R³; —C(O)OR⁴; —C(O)NR⁵R⁶; —S(O)₂R^{3a}; —S(O)R^{4a}; —S(O)₂NR^{5a}R^{6a}; —S(O)(NR^{5a})R^{4a}; —S(NR^{5a})(NR^{6a})R^{3a}; wherein said C₁₋₆alkyl, C₃₋₉ cycloalkyl, C₂₋₆alkenyl, C₅₋₉cycloalkenyl, C₁₋₆heteroalkyl and C₂₋₆heteroalkenyl can be unsubstituted or substituted with one or more substituents selected from C₁₋₆alkyl, C₃₋₉cycloalkyl, C₂₋₆alkenyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆alkyl)₂, —S(O)(NH)C₁₋₆alkyl, —S(O)(NC₁₋₆alkyl)C₁₋₆ alkyl, —S(NH)(NH)C₁₋₆alkyl, —NH₂, —NHC₁₋₆alkyl, —N(C₁₋₆alkyl)₂. In another particular embodiment, R¹ is selected from C₃₋₉cycloalkyl; C₂₋₆alkenyl; C₅₋₉cycloalkenyl; C₁₋₆heteroalkyl; C₂₋₆heteroalkenyl; —C(O)R³; —C(O)OR⁴; —C(O)NR⁵R⁶; —S(O)₂R^{3a}; —S(O)R^{4a}; —S(O)₂NR^{5a}R^{6a}; —S(O)(NR^{5a})R^{4a}; —S(NR^{5a})(NR^{6a})R^{3a}; wherein said C₃₋₉cycloalkyl, C₂-alkenyl, C₅-cycloalkenyl, C₁₋₆heteroalkyl and C₂₋₆heteroalkenyl can be unsubstituted or substituted with one or more substituents selected from C₁₋₆alkyl, C₃₋₉cycloalkyl, C₂₋₆alkenyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆alkyl)₂, —S(O)(NH)C₁₋₆ alkyl, —S(O)(NC₁₋₆alkyl)C₁₋₆alkyl, —S(NH)(NH)C₁₋₆alkyl, —NH₂, —NHC₁₋₆alkyl, —N(C₁₋₆alkyl)₂.

In another particular embodiment, R¹ is selected from C₁₋₆alkyl; —C(O)R³; —C(O)OR⁴; —S(O)₂R^{3a}; wherein said C₁₋₆alkyl can be unsubstituted or substituted with one or more substituents selected from C₁₋₆alkyl, C₃₋₉cycloalkyl, C₂₋₆alkenyl, hydroxyl, ═O, halogen, —SH, ═S, trifluoromethyl, —O—C₁₋₆ alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆alkyl)₂, —S(O)(NH)C₁₋₆ alkyl, —S(O)(NC₁₋₆alkyl)C₁₋₆alkyl, —S(NH)(NH)C₁₋₆alkyl, —NH₂, —NHC₁₋₆alkyl, —N(C₁₋₆alkyl)₂.

In another particular embodiment, R¹ is selected from C₁₋₆alkyl; —C(O)R³; —C(O)OR⁴; —S(O)₂R^{3a}; wherein said C₁₋₆alkyl can be unsubstituted or substituted with one or more substituents selected from hydroxyl, cyano, —C(O)OH, —SO₂C₁₋₆alkyl, —SO₂NHC₁₋₆alkyl, NHC₁₋₆alkyl, —N(C₁₋₆alkyl)₂.

In yet another particular embodiment, R¹ is selected from C₁₋₆alkyl; C₃₋₉cycloalkyl; C₂₋₆alkenyl; C₅₋₉cycloalkenyl; C₁₋₆heteroalkyl; C₂₋₆heteroalkenyl; wherein said C₁₋₆alkyl, C₃₋₉cycloalkyl, C₂₋₆ alkenyl, C₅₋₉cycloalkenyl, C₁₋₆heteroalkyl and C₂₋₆heteroalkenyl can be unsubstituted or substituted with one or more substituents selected from C₁₋₆alkyl, C₃₋₉cycloalkyl, C₂₋₆alkenyl, hydroxyl, halogen, —SH, trifluoromethyl, —O—C₁₋₆alkyl, —OCF₃, cyano, nitro, —C(O)OH, —C(O)OC₁₋₆ alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆alkyl)₂, —S(O)(NH)C₁₋₆alkyl, —S(O)(NC₁₋₆alkyl)C₁₋₆alkyl, —S(NH)(NH)C₁₋₆alkyl, —NH₂, —NHC₁₋₆alkyl, —N(C₁₋₆alkyl)₂.

In yet another particular embodiment, R¹ is selected from C₁₋₆alkyl; C₂₋₆alkenyl; C₁₋₆heteroalkyl; C₂₋₆heteroalkenyl; wherein said C₁₋₆alkyl, C₂₋₆alkenyl, C₁₋₆heteroalkyl and C₂₋₆heteroalkenyl can be unsubstituted or substituted with one or more substituents selected from hydroxyl, halogen, —SH, trifluoromethyl, —O—C₁₋₆alkyl, —OCF₃, cyano, nitro, —C(O)OH, —CONH₂, —CONHC₁₋₆alkyl, —CON(C₁₋₆ alkyl)₂, —SO₂C₁₋₆alkyl, —SO₂NH₂, —SO₂NHC₁₋₆alkyl, —SO₂N(C₁₋₆alkyl)₂, —S(O)(NH)C₁₋₆alkyl, —S(O)(NC₁₋₆alkyl)C₁₋₆alkyl, —S(NH)(NH)C₁₋₆alkyl, —NH₂, —NHC₁₋₆alkyl, —N(C₁₋₆alkyl)₂. In yet another particular embodiment, R¹ is selected from C₁₋₆alkyl; C₂₋₆alkenyl; C₁₋₆heteroalkyl; C₂₋₆ heteroalkenyl; wherein said C₁₋₆alkyl, C₂₋₆alkenyl, C₁₋₆heteroalkyl and C₂₋₆heteroalkenyl is substituted with one or more substituents selected from hydroxyl, halogen, —SH, trifluoromethyl, —O—C₁₋₆alkyl, —OCF₃, cyano, nitro, —C(O)OH, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆alkyl)₂, —S(O)(NH)C₁₋₆alkyl, —S(O)(NC₁₋₆alkyl)C₁₋₆ alkyl, —S(NH)(NH)C₁₋₆alkyl, —NH₂, —NHC₁₋₆alkyl, —N(C₁₋₆alkyl)₂. In still another particular embodiment, R¹ is selected from C₂₋₃alkyl; C₅₋₆alkyl; C₂₋₆alkenyl; C₁₋₆heteroalkyl; C₂₋₆ heteroalkenyl; wherein said C₁₋₆alkyl, C₂₋₆alkenyl, C₁₋₆heteroalkyl and C₂₋₆heteroalkenyl can be unsubstituted or substituted with one or more substituents selected from hydroxyl, halogen, —SH, trifluoromethyl, —O—C₁₋₆alkyl, —OCF₃, cyano, nitro, —C(O)OH, —CONH₂, —CONHC₁₋₆alkyl, —CON(C₁₋₆ alkyl)₂, —SO₂C₁₋₆alkyl, —SO₂NH₂, —SO₂NHC₁₋₆alkyl, —SO₂N(C₁₋₆alkyl)₂, —S(O)(NH)C₁₋₆alkyl, —S(O)(NC₁₋₆alkyl)C₁₋₆alkyl, —S(NH)(NH)C₁₋₆alkyl, —NH₂, —NHC₁₋₆alkyl, —N(C₁₋₆alkyl)₂.

In still another embodiment, R¹ is selected from —C(O)R³; and —S(O)₂R^{3a}; —S(O)R^{4a}; —S(O)₂NR^{5a}R^{6a}; —S(O)(NR^{5a})R^{4a}; —S(NR^{5a})(NR^{6a})R^{3a}. In yet another embodiment, R¹ is selected from —C(O)R³; and —S(O)₂R^{3a}. In still another particular embodiment, R¹ is —C(O)R³. In still another particular embodiment, R¹ is —S(O)₂R^{3a}.

In a particular embodiment of the present disclosure, the compounds have a structure according to the different formulas (I), (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc) and (IX) and any other formulas described herein, more in particular according to the statements, embodiments and aspects described herein, R² is selected from hydrogen; C₁₋₆alkyl; C₃₋₉cycloalkyl; and C₁₋₆heteroalkyl. In yet another particular embodiment, R² is selected from hydrogen; C₁₋₆alkyl; and C₁₋₆heteroalkyl. In yet another particular embodiment, R² is selected from C₁₋₆alkyl; and C₁₋₆heteroalkyl. In yet another particular embodiment, R² is hydrogen. In yet another particular embodiment, R² is C₁₋₆ alkyl. In yet another particular embodiment, R² is selected from hydrogen; and C₁₋₆heteroalkyl. In yet another particular embodiment, R² is selected from methyl, ethyl and propyl. In yet another particular embodiment, R² is selected from ethyl and propyl.

In another particular embodiment, R¹ and R² can be taken together to form a 4-; 5-; or 6-membered heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆ alkyl)₂, —S(O)(NH)C₁₋₆alkyl, —S(O)(NC₁₋₆alkyl)C₁₋₆alkyl, —S(NH)(NH)C₁₋₆alkyl, —NH₂, —NHC₁₋₆ alkyl, —N(C₁₋₆alkyl)₂. In another particular embodiment, R¹ and R² can be taken together to form a 4-membered heterocycle which can be unsubstituted or substituted with one or more substituents selected from C₁₋₆alkyl, C₃₋₉cycloalkyl, C₂₋₆alkenyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆ alkyl)₂, —S(NH)(NH)C₁₋₆alkyl, —S(NC₁₋₆alkyl)(NH)C₁₋₆alkyl, —S(NC₁₋₆alkyl)(NC₁₋₆alkyl)C₁₋₆alkyl, —NH₂, —NHC₁₋₆alkyl, —N(C₁₋₆alkyl)₂.

In a particular embodiment of the present disclosure, the compounds have a structure according to the different formulas (I), (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc) and (IX) and any other formulas described herein, more in particular according to the statements, embodiments and aspects described herein, whereby each R³ and R^{3a} is independently selected from C₁₋₆alkyl; C₃₋₉cycloalkyl; C₂₋₆alkenyl; C₅₋₉cycloalkenyl; C₂₋₆alkynyl; C₅₋₉cycloalkynyl; C₁₋₆heteroalkyl; C₂₋₆ heteroalkenyl; and C₂₋₆heteroalkynyl; wherein said C₁₋₆alkyl, C₃₋₉cycloalkyl, C₂₋₆alkenyl, C₅₋₉ cycloalkenyl, C₂₋₆alkynyl, C₅₋₉cycloalkynyl, C₁₋₆heteroalkyl, C₂₋₆heteroalkenyl and C₂₋₆ heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂. In another particular embodiment, each R³ and R^{3a} is independently selected from C₁₋₆alkyl; C₂₋₆alkenyl; C₂₋₆alkynyl; C₁₋₆heteroalkyl; C₂₋₆heteroalkenyl; and C₂₋₆heteroalkynyl; wherein said C₁₋₆alkyl, C₂₋₆alkenyl, C₂₋₆alkynyl, C₁₋₆ heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂.

In another particular embodiment, each R³ and R^{3a} is independently selected from C₂₋₆alkenyl; C₅₋₉cycloalkenyl; C₂₋₆alkynyl; C₅₋₉cycloalkynyl; C₂₋₆heteroalkenyl; and C₂₋₆heteroalkynyl; wherein said C₂₋₆alkenyl, C₅₋₉cycloalkenyl, C₂₋₆alkynyl, C₅₋₉cycloalkynyl, C₂₋₆heteroalkenyl and C₂₋₆ heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂. In yet another particular embodiment, each R³ and R^{3a} is independently selected from C₂₋₆alkenyl; and C₂₋₆alkynyl; wherein said C₂₋₆alkenyl and C₂₋₆alkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂.

In another particular embodiment, each R³ and R^{3a} is independently selected from methyl; ethyl; propyl; butyl; vinyl; ethynyl; propenyl; propynyl; butenyl; and butynyl; wherein said methyl, ethyl, propyl, butyl, vinyl, ethynyl, propynyl, propenyl, butenyl and butynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH. —NHalkyl, and —N(alkyl)₂. In another particular embodiment, each R³ and R^{3a} is independently selected from vinyl; ethynyl; propenyl; propynyl; butenyl; butynyl. In yet another particular embodiment, each R³ and R^{3a} is independently selected from vinyl; and ethynyl.

In a particular embodiment of the present disclosure, the compounds have a structure according to the different formulas (I), (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc) and (IX) and any other formulas described herein, more in particular according to the statements, embodiments and aspects described herein, whereby each R⁴ and R^{4a} is independently selected from C₁₋₆alkyl; C₃₋₆ cycloalkyl; C₂₋₆alkenyl; C₅₋₉cycloalkenyl; C₂₋₆alkynyl; C₅₋₉cycloalkynyl; C₁₋₆heteroalkyl; C₂₋₆ heteroalkenyl; and C₂₋₆heteroalkynyl; wherein said C₁₋₆alkyl, C₃₋₉cycloalkyl, C₂₋₆alkenyl, C₅₋₉ cycloalkenyl, C₂₋₆alkynyl, C₅₋₉cycloalkynyl, C₁₋₆heteroalkyl, C₂₋₆heteroalkenyl and C₂₋₆ heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂. In another particular embodiment, each R⁴ and R^{4a} is independently selected from C₁₋₆alkyl; C₂₋₆alkenyl; C₂₋₆alkynyl; C₁₋₆heteroalkyl; C₂₋₆heteroalkenyl; and C₂₋₆heteroalkynyl; wherein said C₁₋₆alkyl, C₂₋₆alkenyl, C₂₋₆alkynyl, C₁₋₆ heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂. In another particular embodiment, each R⁴ and R^{4a} is independently selected from C₁₋₆akyl; C₂₋₆alkenyl; C₂₋₆alkynyl; wherein said C₁₋₆alkyl, C₂₋₆alkenyl, and C₂₋₆alkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂. In yet another particular embodiment, each R⁴ and R^{4a} is independently selected from methyl, ethyl, propyl and butyl. In still another particular embodiment, each R⁴ and R^{4a} is tbutyl.

In a particular embodiment of the present disclosure, the compounds have a structure according to the different formulas (I), (II), (la), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc) and (IX) and any other formulas described herein, more in particular according to the statements, embodiments and aspects described herein, whereby each R⁵, R⁵, R^{5b}, R⁵, R^{5a} and R^{5b} is independently selected from hydrogen; C₁₋₆alkyl; C₂₋₆alkenyl; C₂₋₆alkynyl; C₁₋₆heteroalkyl; C₂₋₆heteroalkenyl; and C₂₋₆ heteroalkynyl; wherein said C₁₋₆alkyl, C₂₋₆alkenyl, C₂₋₆alkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂.

In another particular embodiment, each R⁵, R⁵, R^{5b}, R⁶, R^{6a} and R^{6b} is independently selected from hydrogen; C₁₋₆alkyl; C₂₋₆alkenyl and C₂₋₆alkynyl; wherein said C₁₋₆alkyl, C₂₋₆alkenyl and C₂₋₆ alkynyl can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O— alkyl, —OCF₃, —CHF₂, —OCHF₂, cyano, nitro, —C(O)OH; NH₂; —NHalkyl, and —N(alkyl)₂. In a particular embodiment of the present disclosure, the compounds have a structure according to the different formulas (I), (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc) and (IX) and any other formulas described herein, more in particular according to the statements, embodiments and aspects described herein, cycle A is selected from aryl; and heteroaryl; wherein said aryl and heteroaryl can be unsubstituted or substituted with one or more R⁷. In another particular embodiment, cycle A is selected from unsubstituted or substituted with one or more R⁷ aryl. In another particular embodiment, cycle A is selected from unsubstituted or substituted with one or more R⁷ heteroaryl. In another particular embodiment, cycle A is selected from aryl and heteroaryl, wherein said aryl and heteroaryl is substituted with one or more R⁷. In another particular embodiment, cycle A is selected from aryl and heteroaryl, wherein said aryl and heteroaryl is substituted with 1, 2, 3 or 4 R⁷. In another particular embodiment, cycle A is selected from aryl and heteroaryl, wherein said aryl and heteroaryl is substituted with 1, 2 or 3 R⁷. In another particular embodiment, cycle A is selected from aryl and heteroaryl, wherein said aryl and heteroaryl is substituted with 2, 3 or 4 R⁷. In another particular embodiment, cycle A is selected from aryl and heteroaryl, wherein said aryl and heteroaryl is substituted with 3 or 4 R⁷.

In another particular embodiment, cycle A is selected from unsubstituted or substituted with one or more R⁷ phenyl; naphthalenyl; anthracenyl; cyclopropyl; cyclobutyl; cycloheptyl; cyclooctyl; norbornyl; fenchyl; decalinyl; adamantly; triazolyl; pyrazolyl; pyrrolyl; furanyl; imidazolyl; oxazolyl; isoxazolyl; thiazolyl; isothiazolyl; oxadiazolyl; thiadiazolyl; oxatriazolyl; pyrimidyl; pyrazinyl; pyridazinyl; triazinyl; indolyl; indolizinyl; isoindolyl; benzofuranyl; benzothiophenyl; indazolyl; benzimidazolyl; benzoxazolyl; benzisoxazolyl; benzothiazolyl; benzoisothiazolyl; dihydro-benzofuranyl; thienopyridinyl; imidazopyridinyl; benzodioxolyl; quinolinyl; isoquinolinyl; cinnolinyl; quinazolinyl; and quinoxalinyl.

In another particular embodiment, cycle A is selected from unsubstituted or substituted with one or more R⁷ phenyl; naphthalenyl; anthracenyl; triazolyl; pyrazolyl; pyrrolyl; furanyl; imidazolyl; oxazolyl; isoxazolyl; thiazolyl; isothiazolyl; oxadiazolyl; thiadiazolyl; oxatriazolyl; pyrimidyl; pyrazinyl; pyridazinyl; triazinyl; indolyl; indolizinyl; isoindolyl; benzofuranyl; benzothiophenyl; indazolyl; benzimidazolyl; benzoxazolyl; benzisoxazolyl; benzothiazolyl; benzoisothiazolyl; dihydro-benzofuranyl; thienopyridinyl; imidazopyridinyl; benzodioxolyl; quinolinyl; isoquinolinyl; cinnolinyl; quinazolinyl; and quinoxalinyl.

In another particular embodiment, cycle A is selected from unsubstituted or substituted with one or more R⁷ phenyl; pyridinyl; pyrimidyl; pyrazinyl; pyridazinyl; oxazinyl; dioxinyl; thiazinyl; and triazinyl. In another particular embodiment, cycle A is selected from unsubstituted or substituted with one or more R⁷ phenyl; pyridinyl; pyrimidyl; pyrazinyl; and pyridazinyl.

In another particular embodiment, cycle A is substituted with one or more R⁷. In another particular embodiment, cycle A is substituted with 1, 2, 3 or 4 R⁷. In another particular embodiment, cycle A is substituted with 1, 2 or 3 R⁷. In another particular embodiment, cycle A is substituted with 1 or 2 R⁷. In another particular embodiment, cycle A is substituted with 2, 3 or 4 R⁷. In another particular embodiment, cycle A is substituted with 3 or 4 R⁷. In another particular embodiment, cycle A is substituted with 2 R⁷, which are not adjacent to each other (eg 1 R⁷ in ortho and 1 R⁷ in para, or 2 R⁷s in meta and 1 R⁷ in para. In another particular embodiment, cycle A is substituted with 1, 2, 3 or 4 R⁷, of which at least 1 R⁷ is in para-position.

In a particular embodiment of the present disclosure, the compounds have a structure according to the different formulas (I), (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc) and (IX) and any other formulas described herein, more in particular according to the statements, embodiments and aspects described herein, whereby each R⁷ is independently selected from halogen; hydroxyl; sulfhydryl; ═O; ═S; —OZ¹; —SZ¹; —SCF₃; —SF₅; —CF₃; —OCF₃; —CHF₂; —OCHF₂; —NZ³Z⁴; —NZ³C(O)Z¹; cyano; —C(O)Z²; —C(O)OZ¹; —C(O)NZ³Z⁴; C₁₋₆alkyl; C₃₋₉cycloalkyl; C₂₋₆alkenyl; C₂₋₆alkynyl; C₁₋₆ heteroalkyl; C₂₋₆heteroalkenyl; C₂₋₆heteroalkynyl; aryl; heteroaryl; heterocycle; arylC₁₋₆alkyl; arylC₂₋₆alkenyl; arylalkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆ alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl; wherein said C₁₋₆alkyl, C₃₋₉cycloalkyl, C₂₋₆alkenyl, C₂₋₆alkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆ alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂.

In another particular embodiment, each R⁷ is independently selected from halogen; hydroxyl; sulfhydryl; ═O; ═S; —OZ¹; —SZ¹; —SCF₃; —SF₅; —CF₃; —OCF₃; —CHF₂; —OCHF₂; —NZ³Z⁴; —NZ³C(O)Z¹; cyano; —C(O)Z²; —C(O)OZ¹; —C(O)NZ³Z⁴; C₁₋₆alkyl; C₃₋₉cycloalkyl; C₁₋₆heteroalkyl; aryl; heteroaryl; heterocycle; arylC₁₋₆alkyl; arylC₁₋₆heteroalkyl; heteroarylC₁₋₆alkyl; heteroarylC₁₋₆ heteroalkyl; heterocycle-C₁₋₆alkyl; heterocycle-C₁₋₆heteroalkyl; wherein said C₁₋₆alkyl, C₃₋₉cycloalkyl, C₁₋₆heteroalkyl, aryl, heteroaryl, heterocycle, arylC₁₋₆alkyl, arylC₁₋₆heteroalkyl, heteroarylC₁₋₆alkyl, heteroarylC₁₋₆heteroalkyl, heterocycle-C₁₋₆alkyl and heterocycle-C₁₋₆ heteroalkyl can be unsubstituted or substituted with one or more substituents selected from C₁₋₆ alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂.

In another particular embodiment, R⁷ is independently selected from halogen; hydroxyl; sulfhydryl; ═O; ═S; —OZ¹; —SZ¹; —SCF₃; —SF5; —CF₃; —OCF₃; —CHF₂; —OCHF₂; —NZ³Z⁴; —NZ³C(O)Z¹; cyano; —C(O)Z²; —C(O)OZ¹; —C(O)NZ³Z⁴; C₁₋₆alkyl; C₃₋₉cycloalkyl; C₂₋₆alkenyl; C₂₋₆ alkynyl; C₁₆heteroalkyl; C₂₋₆heteroalkenyl; C₂₋₆heteroalkynyl; wherein said C₁₋₆alkyl, C₃₋₉cycloalkyl, C₂₋₆alkenyl, C₂₋₆alkynyl, C₁₋₆heteroalkyl, C₂₋₆heteroalkenyl, C₂₋₆heteroalkynyl, can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂. In another particular embodiment, R⁷ is independently selected from halogen; hydroxyl; sulfhydryl; ═O; ═S; —OZ¹; —SZ¹; —SCF₃; —SF₅; —CF₃; —OCF₃; —CHF₂; —OCHF₂; —NZ³Z⁴; —NZ³C(O)Z¹; cyano; —C(O)NZ³Z⁴; C₁₋₆alkyl; C₃₋₉cycloalkyl; C₂₋₆alkenyl; C₂₋₆alkynyl; C₁₋₆heteroalkyl; C₂₋₆heteroalkenyl; and C₂₋₆ heteroalkynyl.

In another particular embodiment, each R⁷ is independently selected from halogen; hydroxyl; sulfhydryl; ═O; ═S; —OZ¹; —SZ¹; —SCF₃; —SF5; —CF₃; —OCF₃; —CHF₂; —OCHF₂; —NZ³Z⁴; cyano; C₁₋₆alkyl; C₃₋₉cycloalkyl; C₁₋₆heteroalkyl; aryl; heteroaryl; heterocycle; wherein said C₁₋₆ alkyl, C₃₉cycloakyl, C₁₋₆heteroalkyl, aryl, heteroaryl and heterocycle can be unsubstituted or substituted with one or more substituents selected from C₁₋₆alkyl, C₃₋₉cycloalkyl, C₂₋₆alkenyl, C₂₋₆ alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂.

In another particular embodiment, each R⁷ is independently selected from F; Cl; Br; I; hydroxyl; sulfhydryl; ═O; ═S; —OZ¹; —SZ¹; —SCF₃; —SF₅; —CF₃; —OCF₃; —CHF₂; —OCHF₂; —NZ³Z⁴; cyano; and C₁₋₆alkyl; wherein said C₁₋₆alkyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂.

In another particular embodiment, each R⁷ is independently selected from F; Br; I; hydroxyl; sulfhydryl; ═O; ═S; —SZ¹; —SCF₃; —SF₅; —CF₃; —OCF₃; —CHF₂; —OCHF₂; —NZ³Z⁴; an d cyano.

In a particular embodiment of the present disclosure, the compounds have a structure according to the different formulas (I), (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc) and (IX) and any other formulas described herein, more in particular according to the statements, embodiments and aspects described herein, whereby—X¹ is selected from CR⁸; N; and NR^{8a}; whereby X¹ can only be NR^{5a} when X² and/or X⁴ are C═O or C═S;
X² is selected from CR⁹; N; and NR^{9a}; whereby X² can only be NR^{9a} when X¹ and/or X³ are C═O or C—SH;
X³ is selected from CH; and N;
X⁴ is selected from C^{H}; and N;
whereby maximally 2 of X¹, X², X³ and X⁴ can be a N (selected from N and NR^{8a} for X¹, from N and NR^{9a} for X², from N for X³, and from N for X⁴ respectively) at the same time; In a particular embodiment,
X¹ is selected from CR⁸; N; and NR^{8a}; whereby X¹ can only be NR^{8a} when X² and/or X⁴ are C═O or C═S;
X² is selected from CR⁹; N;
X³ is selected from CH; N;
X⁴ is selected from CH; N;
whereby maximally 2 of X¹, X², X³ and X⁴ can be a N (selected from N and NR^{8a} for X¹, from N and NR^{9a} for X², from N for X³, and from N for X⁴ respectively) at the same time.

In another particular embodiment X¹ is CR⁸; X² is CR⁹; X³ is CH; and X⁴ is CH.

In a particular embodiment, X¹ is selected from CR⁸; N; and NR^{8a}; whereby X¹ can only be NR^{8a} when X² and/or X⁴ are C═O or C═S; X² is CR⁹; X³ is CH; and X⁴ is CH.

In another particular embodiment, X¹ is CR⁸; X² is selected from CR⁹; and N, whereby X² can only be NR^{9a} when X¹ and/or X³ are C═O or C—SH; X³ is CH; and X⁴ is CH. In a particular embodiment of the present disclosure, the compounds have a structure according to the different formulas (I), (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc) and (IX) and any other formulas described herein, more in particular according to the statements, embodiments and aspects described herein, whereby each R⁸ and R⁹ are independently selected from hydrogen; halogen; hydroxyl; sulfhydryl; ═O; ═S; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; —NZ^{3a}Z^{4a} —NZ^{3a}S(O)₂Z^{1a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; C₁₋₆alkyl; C₃₋₉cycloakyl; C₂₋₆alkenyl; C₂₋₆alkynyl; C₁₋₆heteroalkyl; C₂₋₆ heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆akyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆-akyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl; wherein said C₁₋₆alkyl, C₃₋₉cycloakyl, C₂₋₆alkenyl, C₂₋₆alkynyl, C₁₋₆ heteroalkyl, C₂₋₆heteroalkenyl, C₂₋₆heteroalkynyl, aryl, heteroaryl, heterocycle, arylC₁₋₆alkyl, arylC₂₋₆alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆ alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂.

In another particular embodiment, each R⁸ and R⁹ are independently selected from hydrogen; halogen; ═O; ═S; —OZ^{1a}; —SZ^{1a}; —SCF₃; —SF₅; —CF₃; —OCF₃; —CHF₂; —OCHF₂; —NZ^{3a}Z^{4a} —NZ^{3a}S(O)₂Z^{1a}; —NZ^{3a}C(O)Z^{1a}; —NZ^{3a}C(O)NZ^{3a}Z^{4a}; cyano; —C(O)OZ^{1a}; —C(O)NZ^{3a}Z^{4a}; C₁₋₆alkyl; C₃₋₉cycloakyl; C₁₋₆heteroalkyl; aryl; heteroaryl; heterocycle; arylC₁₋₆alkyl; arylC₁₋₆heteroalkyl; heteroarylC₁₋₆alkyl; heteroarylC₁₋₆heteroalkyl; heterocycle-C₁₋₆alkyl; and heterocycle-C₁₋₆heteroalkyl; wherein said C₁₋₆alkyl, C₃₋₉cycloakyl, C₁₋₆heteroalkyl, aryl, heteroaryl, heterocycle, arylC₁₋₆alkyl, arylC₁₋₆heteroalkyl, heteroarylC₁₋₆alkyl, heteroarylC₁₋₆ heteroalkyl, heterocycle-C₁₋₆-akyl and heterocycle-C₁₋₆heteroalkyl can be unsubstituted or substituted with one or more substituents selected from C₁₋₆alkyl, C₃₋₉cycloalkyl, C₂₋₆alkenyl, C₂₋₆ alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂.

In yet another particular embodiment, each R³ and R⁹ are independently selected from hydrogen; halogen; ═O; —OZ^{1a}; —CF₃; —OCF₃; —CHF₂; —OCHF₂; —NZ^{3a}Z^{4a}; —NZ^{3a}S(O)₂Z^{1a} —NZ^{3a}C(O)Z^{1a}; —NZ^{3a}C(O)NZ^{3a}Z^{4a}; cyano; —C(O)OZ^{1a}; —C(O)NZ^{3a}Z^{4a}; C₁₋₆alkyl; aryl; heteroaryl; arylC₁₋₆alkyl; heteroarylC₁₋₆alkyl; and heterocycle-C₁₋₆alkyl; wherein said C₁₋₆alkyl, aryl, heteroaryl, arylC₁₋₆alkyl, heteroarylC₁₋₆alkyl and heterocycle-C₁₋₆alkyl can be unsubstituted or substituted with one or more substituents selected from C₁₋₆alkyl, C₃₋₆cycloalkyl, hydroxyl, ═O, halogen, —SH, ═S, —CF₃, —O—C₁₋₆alkyl, —OCF₃, —CHF₂; —OCHF₂, cyano, —C(O)OH, —NH₂, —NHC₁₋₆alkyl, and —N(C₁₋₆alkyl)₂.

In yet another particular embodiment, R³ is selected from halogen; ═O; —OZ^{1a}; —CF₃; —OCF₃; —CHF₂; —OCHF₂; —NZ^{3a}Z^{4a}; —NZ^{3a}S(O)₂Z^{1a}; —NZ^{3a}C(O)Z^{1a}; —NZ^{3a}C(O)NZ^{3a}Z⁴a cyano; —C(O)OZ^{1a}; —C(O)NZ^{3a}Z^{4a}; C₁₋₆alkyl; aryl; heteroaryl; arylC₁₋₆alkyl; heteroarylC₁₋₆alkyl; and heterocycle-C₁₋₆alkyl; wherein said C₁₋₆alkyl, aryl, heteroaryl, arylC₁₋₆alkyl, heteroarylC₁₋₆ alkyl and heterocycle-C₁₋₆alkyl can be unsubstituted or substituted with one or more substituents selected from C₁₋₆alkyl, C₃₋₉cycloalkyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂. In still another particular embodiment, R³ is selected from halogen; ═O; —OZ^{1a}; —NZ^{3a}Z^{4a}; —NZ^{3a}C(O)Z^{1a}; cyano; —C(O)OZ^{1a}; —C(O)NZ^{3a}Z^{4a}; C₁₋₆alkyl; heteroaryl; arylC₁₋₆alkyl; heteroarylC₁₋₆alkyl; and heterocycle-C₁₋₆alkyl; wherein said C₁₋₆alkyl, heteroaryl, arylC₁₋₆alkyl, heteroarylC₁₋₆alkyl and heterocycle-C₁₋₆alkyl can be unsubstituted or substituted with one or more substituents selected from C₁₋₆alkyl, C₃₋₉cycloalkyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂.

In yet another particular embodiment, R⁹ is selected from halogen; ═O; —OZ^{1a}; —CF₃; —OCF₃; —CHF₂; —OCHF₂; —NZ^{3a}Z^{4a}; —NZ^{3a}S(O)₂Z^{1a}; —NZ^{3a}C(O)Z^{1a}; —NZ^{3a}C(O)NZ^{3a}Z⁴a cyano; —C(O)OZ^{1a}; —C(O)NZ^{3a}Z^{4a}; C₁₋₆alkyl; aryl; heteroaryl; arylC₁₋₆alkyl; heteroarylC₁₋₆alkyl; and heterocycle-C₁₋₆alkyl; wherein said C₁₋₆alkyl, aryl, heteroaryl, arylC₁₋₆alkyl, heteroarylC₁₋₆alkyl and heterocycle-C₁₋₆-akyl can be unsubstituted or substituted with one or more substituents selected from C₁₋₆alkyl, C₃₋₉cycloalkyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂. In still another particular embodiment, R⁹ is selected from hydrogen; halogen; ═O; —CF₃; —OCF₃; —CHF₂; —OCHF₂; —NZ^{3a}Z^{4a} —NZ^{3a}S(O)₂Z^{1a}; —NZ^{3a}C(O)Z^{1a}; —NZ^{3a}C(O)NZ^{3a}Z^{4a}; —C(O)OZ^{1a}; —C(O)NZ^{3a}Z^{4a}; C₁₋₆alkyl; aryl; heteroaryl; arylC₁₋₆alkyl; heteroarylC₁₋₆alkyl; and heterocycle-C₁₋₆ alkyl; wherein said C₁₋₆alkyl, aryl, heteroaryl, arylC₁₋₆alkyl, heteroarylC₁₋₆alkyl and heterocycle-C₁₋₆alkyl can be unsubstituted or substituted with one or more substituents selected from C₁₋₆ alkyl, C₃₋₉cycloalkyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂.

In a particular embodiment of the present disclosure, the compounds have a structure according to the different formulas (I), (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc) and (IX) and any other formulas described herein, more in particular according to the statements, embodiments and aspects described herein, whereby each R^{8a} and R^{9a} are independently selected from hydrogen; C₁₋₆akyl; C₃₋₉cycloakyl; C₁₋₆heteroalkyl; aryl; heteroaryl; heterocycle; arylC₁₋₆alkyl; heteroarylC₁₋₆akyl; heterocycle-C₁₋₆-akyl; wherein said C₁₋₆alkyl, C₃₋₉cycloakyl, aryl, heteroaryl, heterocycle, arylC₁₋₆alkyl, heteroarylC₁₋₆alkyl and heterocycle-C₁₋₆alkyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂.

In yet another particular embodiment each R^{5a} and R^{9a} are independently selected from hydrogen; and C₁₋₆alkyl; wherein said C₁₋₆alkyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂.

In a particular embodiment of the present disclosure, the compounds have a structure according to the different formulas (I), (II), (la), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc) and (IX) and any other formulas described herein, more in particular according to the statements, embodiments and aspects described herein, whereby each Z¹ and Z^{1a} is independently selected from C₁₋₆alkyl; C₃₋₉cycloakyl; and C₁₋₆heteroalkyl; wherein said C₁₋₆alkyl, C₃₋₉cycloakyl and C₁₋₆heteroalkyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂.

In a particular embodiment of the present disclosure, the compounds have a structure according to the different formulas (I), (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc) and (IX) and any other formulas described herein, more in particular according to the statements, embodiments and aspects described herein, whereby each Z² and Z^{2a} is independently selected from hydroxyl; and C₁₋₆ heteroalkyl.

In a particular embodiment of the present disclosure, the compounds have a structure according to the different formulas (I), (II), (la), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc) and (IX) and any other formulas described herein, more in particular according to the statements, embodiments and aspects described herein, whereby each Z³, Z^{3a}, Z⁴, and Z^{4a} is independently selected from hydrogen; C₁₋₆alkyl; C₃₋₉cycloakyl; C₁₋₆heteroalkyl; aryl; heteroaryl; and heterocycle; wherein said C₁₋₆alkyl, C₃₋₉cycloakyl, C₁₋₆heteroalkyl, aryl, heteroaryl, and heterocycle can be unsubstituted or substituted with one or more substituents selected from C₁₋₆alkyl, C₃₋₉cycloalkyl, C₂₋₆alkenyl, C₂₋₆ akynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl).

The compounds of the disclosure are compounds of formula (X), (Xa), (Xb), (Xc), (Xd), (Xe), (X′), (Xa′), (Xb′), (Xc′), (Xd′) and (Xe′) and any subgroup thereof as described herein, a stereo-isomeric form, a tautomer, a salt (in particular a pharmaceutically acceptable salt), solvate, polymorph and/or prodrug thereof,

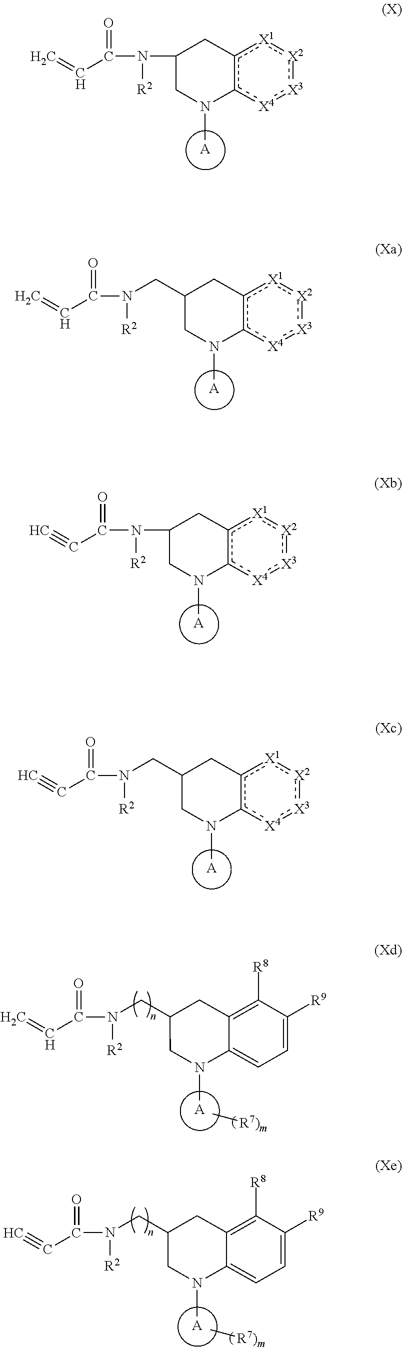

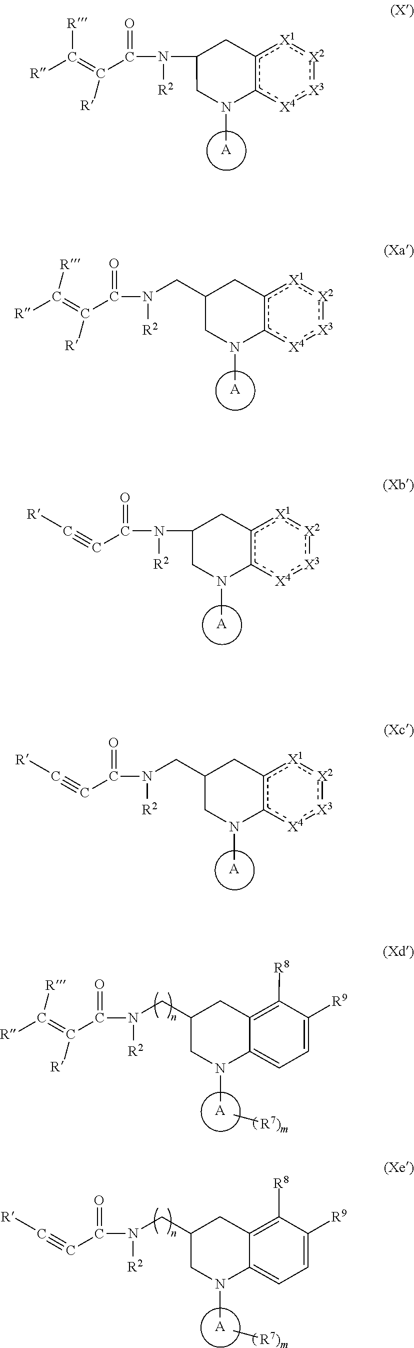

wherein:
n is selected from 0; 1; and 2;
each — represents an optional double bond, whereby maximally 3 —are a double bond at the same time;
R¹ is selected from alkyl; cycloalkyl; alkenyl; cycloalkenyl; alkynyl; cycloalkynyl; heteroalkyl; heteroalkenyl; heteroalkynyl; —C(O)H; —C(O)R³; —C(O)OR⁴; —C(O)NR⁵R⁶; —S(O)₂R^{3a}; —S(O)R^{4a}; —S(O)₂NR^{5a}R^{6a}; —S(O)(NR^{5a})R^{4a}; —S(NR^{5a})(NR^{6a})R^{3a}; and —P(O)R^{5b}R^{6b};
   wherein said alkyl, cycloalkyl, alkenyl, cycloalkenyl, alkynyl, cycloalkynyl, heteroalkyl, heteroalkenyl and heteroalkynyl can be unsubstituted or substituted with one or more substituents selected from alkyl, cycloalkyl, alkenyl, alkynyl, hydroxyl, ═O, halogen, —SH, ═S, trifluoromethyl, —O-alkyl, —OCF₃, cyano, nitro, —C(O)OH, —C(O)Oalkyl, —C(O)alkyl, —CONH₂, —CONHalkyl, —CON(alkyl)₂, —SO₂alkyl, —SO₂NH₂, —SO₂NHalkyl, —SO₂N(alkyl)₂, —S(O)(NH)alkyl, —S(O)(Nalkyl)alkyl, —S(NH)(NH)alkyl, —NH₂, —NHalkyl, —N(alkyl)₂;
R² is selected from hydrogen; alkyl; cycloalkyl; and heteroalkyl;
R¹ and R² can be taken together to form a (4-; 5-; 6- or 7-membered) heterocycle which can be unsubstituted or substituted with one or more substituents selected from alkyl, cycloalkyl, alkenyl, alkynyl, hydroxyl, ═O, halogen, —SH, ═S, trifluoromethyl, —O-alkyl, —OCF₃, cyano, nitro, —C(O)OH, —C(O)Oalkyl, —C(O)alkyl, —CONH₂, —CONHalkyl, —CON(alkyl)₂, —SO₂alkyl, —SO₂NH₂, —SO₂NHalkyl, —SO₂N(alkyl)₂, —S(O)(NH)alkyl, —S(O)(Nalkyl)alkyl, —S(NH)(NH)alkyl, —NH₂, —NHalkyl, —N(alkyl)₂;
each R³ and R^{3a} is independently selected from hydroxyl; alkyl; cycloalkyl; alkenyl; cycloalkenyl; alkynyl; cycloalkynyl; heteroalkyl; heteroalkenyl; and heteroalkynyl;
   wherein said alkyl, cycloalkyl, alkenyl, cycloalkenyl, alkynyl, cycloalkynyl, heteroalkyl, heteroalkenyl and heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁴ and R^{4a} is independently selected from alkyl; alkenyl; cycloalkenyl; alkynyl; cycloalkyl; cycloalkynyl; heteroalkyl; heteroalkenyl; and heteroalkynyl;
   wherein said alkyl, cycloalkyl, alkenyl, cycloalkenyl, alkynyl, cycloalkynyl, heteroalkyl, heteroalkenyl and heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁵, R^{5a}, R^{5b}, R⁶, R^{6a} and R^{6b} is independently selected from hydrogen; alkyl; cycloalkyl; alkenyl; cycloalkenyl; alkynyl; cycloalkynyl; heteroalkyl; heteroalkenyl; and heteroalkynyl;
   wherein said alkyl, cycloalkyl, alkenyl, cycloalkenyl, alkynyl, cycloalkynyl, heteroalkyl, heteroalkenyl and heteroalkynyl can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
   and wherein each R⁵ and R⁶ or R^{5a} and R^{6a} can be taken together in order to form a (4-, 5-, 6-, or 7-membered) heterocycle which can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂.
cycle A is selected from aryl; heteroaryl; cycloalkyl; and heterocycle;
   wherein said aryl, heteroaryl, cycloalkyl and heterocycle can be unsubstituted or substituted with one or more R⁷;
each R⁷ is independently selected from halogen; hydroxyl; sulfhydryl; ═O; ═S; —OZ¹; —SZ¹; —SCF₃; —SF₅; —CF₃; —OCF₃; —CHF₂; —OCHF₂; —NZ³Z⁴; —NZ³C(O)Z¹; cyano; —C(O)Z²; —C(O)OZ¹; —C(O)NZ³Z⁴; alkyl; cycloakyl; alkenyl; alkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; and heterocycle-heteroalkynyl;
   wherein said alkyl, cycloakyl, alkenyl, alkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂; —OCHF₂, cyano, nitro, —C(O)OH, —NH₂, —NHalkyl, and —N(alkyl)₂;
X¹ is selected from CR⁸; N; and NR^{8a}; whereby X¹ can only be NR^{8a} when X² and/or X⁴ are C═O or C═S;
X² is selected from CR⁹; N; and NR^{9a}; whereby X² can only be NR^{9a} when X¹ and/or X³ are C═O or C—SH;
X³ is selected from CH; and N;
X⁴ is selected from CH; and N;
whereby maximally 2 of X¹, X², X³ and X⁴ can be a N (selected from N and NR^{8a} for X¹, from N and NR^{9a} for X², from N for X³, and from N for X⁴ respectively) at the same time;
each R⁸ and R⁹ are independently selected from hydrogen; halogen; hydroxyl; sulfhydryl; ═O; ═S; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{1a} —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; alkyl; cycloakyl; alkenyl; alkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; and heterocycle-heteroalkynyl;
   wherein said alkyl, cycloakyl, alkenyl, alkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂; —OCHF₂, cyano, nitro, —C(O)OH, —NH₂, —NHalkyl, and —N(alkyl)₂;
each R^{8a} and R^{9a} are independently selected from hydrogen; hydroxyl; sulfhydryl; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{2a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; alkyl; cycloakyl; alkenyl; alkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; and heterocycle-heteroalkynyl;
   wherein said alkyl, cycloakyl, alkenyl, alkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂; —OCHF₂, cyano, nitro, —C(O)OH, —NH₂, —NHalkyl, and —N(alkyl)₂;
each R″, R″″ and R″″ is independently selected from halogen, hydroxyl, alkyl; cycloalkyl; alkenyl; cycloalkenyl; alkynyl; cycloalkynyl; heteroalkyl; heteroalkenyl; and heteroalkynyl;
   wherein said alkyl, cycloalkyl, alkenyl, cycloalkenyl, alkynyl, cycloalkynyl, heteroalkyl, heteroalkenyl and heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each Z¹ and Z^{1a} is independently selected from alkyl; alkenyl; alkynyl; cycloalkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; or heterocycle-heteroalkynyl;
   wherein said alkyl, cycloalkyl, alkenyl, alkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each Z² and Z^{2a} is independently selected from hydroxyl; alkyl; cycloalkyl; alkenyl; alkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; or heterocycle-heteroalkynyl;
   wherein said alkyl, cycloalkyl, alkenyl, alkynyl, heteroalkyl, heteroalkenyl, heteroalkynyl, aryl, heteroaryl, heterocycle, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each Z³, Z^{3a}, Z⁴, and Z^{4a} is independently selected from hydrogen; alkyl; cycloalkyl; alkenyl; alkynyl; heteroalkyl; heteroalkenyl; heteroalkynyl; aryl; heteroaryl; heterocycle; arylalkyl; arylalkenyl; arylalkynyl; arylheteroalkyl; arylheteroalkenyl; arylheteroalkynyl; heteroarylalkyl; heteroarylalkenyl; heteroarylalkynyl; heteroarylheteroalkyl; heteroarylheteroalkenyl; heteroarylheteroalkynyl; heterocycle-alkyl; heterocycle-alkenyl; heterocycle-alkynyl; heterocycle-heteroalkyl; heterocycle-heteroalkenyl; or heterocycle-heteroalkynyl;
   wherein said alkyl, cycloalkyl, alkenyl, alkynyl, heteroalkyl, heteroalkenyl, heteroalkynyl, aryl, heteroaryl, heterocycle, cycloalkyl, arylalkyl, arylalkenyl, arylalkynyl, arylheteroalkyl, arylheteroalkenyl, arylheteroalkynyl, heteroarylalkyl, heteroarylalkenyl, heteroarylalkynyl, heteroarylheteroalkyl, heteroarylheteroalkenyl, heteroarylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
   and wherein each Z³ and Z⁴ or Z^{3a} and Z^{4a} can be taken together in order to form a (4-, 5-, 6-, or 7-membered) heterocycle which can be unsubstituted or substituted with one or more substituents selected from alkyl, cycloalkyl, alkenylalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂.

In a particular embodiment, the compounds of the disclosure are selected from the compounds listed in Table 1.

In a particular embodiment of the present disclosure, the compounds have a structure according to the formulas provided herein selected from (I), (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc), (IX), (X), (Xa), (Xb), (Xc), (Xd) and (Xe) or other formulas, aspects, statements or embodiments herein, provided that the compounds are not selected from:
Acetamide, N-[1-(1-cyclopentyl-1H-tetrazol-5-yl)-1,2,3,4-tetrahydro-3quinolinyl]-;
Acetamide, N-[(1,2,3,4-tetrahydro-1-phenyl-3-quinolinyl)methyl]-;
Propanamide, N-(1,2,3,4-tetrahydro-1-phenyl-3-quinolinyl)-;
Cyclopropanecarboxamide, N-[1,2,3,4-tetrahydro-1-(3-methoxyphenyl)-3-quinolinyl]-;
Cyclohexaneacetamide, N-(6-cyano-1-cyclohexyl-1,2,3,4-tetrahydro-3-quinolinyl)-;
Carbamic acid, N-[1-(3,4-dichlorophenyl)-1,2,3,4-tetrahydro-3-quinolinyl]-, 1,1-dimethylethyl ester; Carbamic acid, N-[1-(3,4-dichlorophenyl)-1,2,3,4-tetrahydro-3-quinolinyl]—N-methyl-, 1,1-dimethylethyl ester; Carbamic acid, N-[(3S)-1-(3,4-dichlorophenyl)-1,2,3,4-tetrahydro-3-quinolinyl]-, 1,1-dimethylethyl ester; Carbamic acid, N-[(3R)-1-(3,4-dichlorophenyl)-1,2,3,4-tetrahydro-3-quinolinyl]-, 1,1-dimethylethyl ester; Carbamic acid, N-(1,2,3,4-tetrahydro-1-phenyl-3-quinolinyl)-, 1,1-dimethylethyl ester;
compounds wherein cycle A is N-[1-[1-(2,2-dimethylpropyl)-2,3-dihydro-3-methyl-2-oxo-1Himidazo[4,5-b]pyridin-5-yl] or N-[1-[1-[(2,2-difluoro-1-methylcyclopropyl)methyl]-2,3-dihydro-3-methyl-2-oxo-1H-imidazo[4,5-b]pyridin-5-yl]; and
compounds wherein R¹ is methyl or tert-butyl.

In a particular embodiment of the present disclosure, the compounds have a structure according to the (I), (II), (IIa), (IIb), (IIc), (IId), (IIe), (IIf), (III), (IIIa), (IIIb), (IIIc), (IIId), (IIIe), (IIIf), (Illg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc), (IX), (X), (Xa), (Xb), (Xc), (Xd) and (Xe) or other formulas, aspects, statements or other embodiments herein, provided that n is not 0; or n is not 1; or n is not 2; or n is not 1 and 2; or n is not 0 and 2.

In another particular embodiment of the present disclosure, the compounds have a structure according to (I), (II), (la), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc), (IX), (X), (Xa), (Xb), (Xc), (Xd) and (Xe) or other formulas, aspects, statements or embodiments herein, provided that:
R¹ is not selected from —C(O)methyl, —C(O)ethyl, —C(O)cyclopropyl, —C(O)cyclobutyl, —C(O)cyclopentyl, —C(O)cyclohexyl, —C(O)—CH₂-cyclohexyl, —C(O)t-butyl, —C(O)CF₃, —C(O)p-methyl-phenyl, —C(O)pyrazin-2yl, —C(O)₅-trifluoromethyl-pyrazol-3-yl-, C(O)phenyl, —C(O)thiazol-4-yl, —C(O)pyridin-3-yl, —C(O)pyridin-4-yl, —C(O)-pyrazol-1-yl, —C(O)-pyrazol-3-yl, —C(O)-pyrazol-4-yl, or —C(O)imidazol-2-yl; or
R¹ is not selected from —C(O)alkyl, —C(O)cycloalkyl, —C(O)—CH₂-cycloalkyl, —C(O)phenyl, —C(O)pyrazinyl, —C(O)pyrazolyl, —C(O)CF₃, —C(O)thiazolyl, —C(O)pyridinyl, —C(O)-pyrazolyl, or —C(O)imidazolyl; or
R¹ is not selected from —C(O)R³; or
R¹ is not selected from —S(O)₂R^{3a}; or
R¹ is not selected from alkyl; more in particular R¹ is not selected from C₁₋₆alkyl; yet more in particular R¹ is not selected from C₁₋₄alkyl; still more in particular R¹ is not selected from methyl and t-butyl; or
R¹ is not methyl when R² is methyl; or R¹ and R² are not selected from methyl at the same time.

In a particular embodiment of the present disclosure, the compounds have a structure according to (I), (II), (IIa), (IIb), (IIc), (IId), (Ile), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc), (IX), (X), (Xa), (Xb), (Xc), (Xd) and (Xe) or other formulas, aspects, statements or other embodiments herein, provided that: R² is not hydrogen; or R² is not methyl; or R² is not methyl when R¹ is methyl; or R² is not alkyl.

In a particular embodiment of the present disclosure, the compounds have a structure according to formulas (I), (II), (la), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc), (IX), (X), (Xa), (Xb), (Xc), (Xd) and (Xe) or other formulas, aspects, statements or other embodiments herein, provided that:
R³ or R^{3a} are independently not selected from methyl, ethyl, -cyclopropyl, cyclobutyl, cyclopentyl, cyclohexyl, t-butyl, —CH₂-cyclohexyl, —CF₃, p-methyl-phenyl, pyrazin-2yl, 5-trifluoromethyl-pyrazol-3-yl-, phenyl, thiazol-4-yl, pyridin-3-yl, pyridin-4)yl, -pyrazol-1-yl, pyrazol-3-yl, -pyrazol-4-yl, or imidazol-2-yl; or
R³ or R^{3a} are independently not selected from alkyl, cycloalkyl, —CH₂-cycloalkyl, phenyl, pyrazinyl, pyrazolyl, CF₃, thiazolyl, pyridinyl, -pyrazolyl, or imidazolyl; or
R³ or R^{3a} are independently not selected from alkyl, cycloalkyl and CF₃;
R³ or R^{3a} are independently not selected from CF₃ and methyl; or
R³ or R^{3a} are independently not selected from alkyl; or
R³ or R^{3a} are independently not selected from CF₃.

In a particular embodiment of the present disclosure, the compounds have a structure according to formulas (I), (II), (la), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc), (IX), (X), (Xa), (Xb), (Xc), (Xd) and (Xe) or other formulas, aspects, statements or other embodiments herein, provided that R⁴ or R^{4a} is not t-butyl.

In a particular embodiment of the present disclosure, the compounds have a structure according to formulas (I), (II), (la), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc), (IX), (X), (Xa), (Xb), (Xc), (Xd) and (Xe) or other formulas, aspects, statements or other embodiments herein, provided that:
cycle A is not selected from meta-methoxy-phenyl or para-methoxy-phenyl; or
cycle A is not selected from meta-alkoxy-phenyl or para-alkoxy-phenyl; or
cycle A is not selected from meta-substituted phenyl or para-substituted-phenyl; or
cycle A is not phenyl; or cycle A is not aryl; or
cycle A is not tetrazolyl; or cycle A is not tetrazolyl substituted with cycloalkyl; or cycle A is not 1-cyclopentyl-tetrazol-5-yl; or cycle A is not a 5-membered heteroaryl; or cycle A is not heteroaryl;
or cycle A is not a bicyclic heteroaryl; or
cycle A is not di-chloro-phenyl; more in particular cycle A is not 3,4-dichloro-phenyl; or
cycle A is not unsubstituted pyridinyl; or cycle A is not pyridinyl; or
cycle A is not para-ethoxy-phenyl; or
cycle A is not unsubstituted cycloalkyl; or more in particular cycle A is not unsubstituted cyclohexyl or cyclopentyl; or
cycle A is not 2-Me-thien-3-yl; more in particular is not thienyl; or
cycle A is not tetrazolyl substituted with cycloalkyl; or cycle A is not 1-cyclopentyl-tetrazol-5-yl;
or cycle A is not a 5-membered heteroaryl; or cycle A is not heteroaryl; or cycle A is not a bicyclic heteroaryl;
cycle A is not cyclohexyl or cycle A is not cycloalkyl; or
cycle A is not hexahydro-2,4,6-trioxo-5-pyrimidinyl; or
cycle A is not unsubstituted with R⁷;
cycle A is not 2,3-dihydro-2-oxo-1Himidazo[4,5-b]pyridin-5-yl; yet more in particular cycle A is not 2,3-dihydro-2-oxo-1Himidazo[4,5-b]pyridinyl; still more in particular cycle A is not 2,3-dihydro-1 Himidazo[4,5-b]pyridinyl.

In a particular embodiment of the present disclosure, the compounds have a structure according to formulas (I), (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc), (IX), (X), (Xa), (Xb), (Xc), (Xd), (Xe), (X′) and (X″) or other formulas, aspects, statements or other embodiments herein, provided that R⁷ is not methoxy or ethoxy; or R⁷ is not alkoxy; or R⁷ is not cyclopentyl; or R⁷ is not cycloalkyl; or R⁷ is not methyl or pentyl; or R⁷ is not alkyl; or R⁷ is not unsubstituted or substituted methylcyclopropyl; or R⁷ is not Cl; or R⁷ is not halogen; or R⁷ is not selected from alkyl, cycloalkyl and alkoxy; or R⁷ is not selected from alkyl, cycloalkyl, alkoxy and Cl; or R⁷ is not selected from alkyl, cycloalkyl, alkoxy and halogen. In a particular embodiment, 2 R⁷s are not adjacent to each other. In a particular embodiment, R⁷ is not present in ortho position; or yet more in particular R⁷ is not present in meta position; or still more in particular R⁷ is not present in para position.

In a particular embodiment of the present disclosure, the compounds have a structure according to formulas (I), (II), (IIa), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc), (IX), (X), (Xa), (Xb), (Xc), (Xd) and (Xe) or other formulas, aspects, statements or other embodiments herein, provided that:
one or both of R⁸ and R⁹ is independently not cyano; more in particular R⁹ is not cyano; or
one or both of R⁸ and R⁹ is independently not alkyl; or
one or both of R⁸ and R⁹ is independently not halogen; or
one or both of R⁸ and R⁹ is independently not heteroalkyl; more in particular R⁹ is not heteroalkyl; or
one or both of R⁸ and R⁹ is independently not —OMe; more in particular R⁹ is not —OMe.

The present compounds used in the current disclosure may also exist in their stereochemically isomeric form, defining all possible compounds made up of the same atoms bonded by the same sequence of bonds but having different three-dimensional structures, which are not interchangeable. Unless otherwise mentioned or indicated, the chemical designation of compounds encompasses the mixture of all possible stereochemically isomeric forms, which said compounds might possess.

Said mixture may contain all diastereomers and/or enantiomers of the basic molecular structure of said compound. All stereochemically isomeric forms of the compounds used in the present invention either in pure form or in admixture with each other are intended to be embraced within the scope of the present disclosure including any racemic mixtures or racemates.

The compounds of the present disclosure may have at least one chiral carbon atom as indicated in the figure below for formula (I) by the carbon atom labelled with

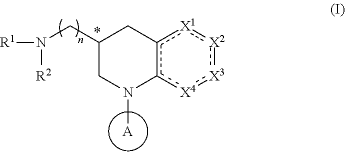

Due to the presence of said chiral carbon atom, a compound of the disclosure such as of formula (I) can be the (R)-enantiomer, the (S)-enantiomer, the racemic form, or any possible combination of the two individual enantiomers in any ratio. When the absolute (R)- or (S)-configuration of an enantiomer is not known, this enantiomer can also be identified by indicating whether the enantiomer is dextrorotatory (+)- or levorotatory (−)-after measuring the specific optical rotation of said particular enantiomer.

An aspect of the present disclosure relates to a first group of compounds of formulas (I), (II), (IIa), (IIb), (IIc), (IId), (Ile), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc), (IX), (X), (Xa), (Xb), (Xc), (Xd) and (Xe) wherein the compounds have the (+) specific rotation.

A further aspect of the present disclosure relates to a second ground of compounds of formulas (I), (II), (la), (IIb), (IIc), (IId), (IIe), (IIf), (III), (IIIa), (IIIb), (IIIc), (IIId), (IIIe), (IIIf), (IIIg), (IV), (IVa), (IVb), (IVc), (IVd), (IVe), (IVf), (IVg), (V), (Va), (Vb), (Vc), (Vd), (Ve), (VI), (VIa), (VII), (VIIa), (VIIb), (VIII), (VIIIa), (VIIIb), (VIIIc), (IX), (X), (Xa), (Xb), (Xc), (Xd) and (Xe) wherein the compounds of formula (I) have the (−) specific rotation.

More generally, the disclosure relates to the compounds of the formulae described herein and embodiments, statements and aspects thereof being useful as agents having biological activity or as diagnostic agents. Any of the uses mentioned with respect to the present disclosure may be restricted to a non-medical use, a non-therapeutic use, a non-diagnostic use, or exclusively an in vitro use, or a use related to cells remote from an animal.

Compounds of the present disclosure are small molecule YAP/TAZ-TEAD inhibitors. Small molecule YAP/TAZ-TEAD inhibitors are useful, e.g., for the treatment of cancer, including with no limitations, lung cancer, breast cancer, head and neck cancer, oesophageal cancer, kidney cancer, bladder cancer, colon cancer, ovarian cancer, cervical cancer, endometrial cancer, liver cancer (including but not limited to cholangiocarcinoma), skin cancer, pancreatic cancer, gastric cancer, brain cancer and prostate cancer, mesotheliomas, and/or sarcomas. In other embodiments, small molecule YAP/TAZ-TEAD inhibitors are useful for the treatment of cancers characterized by squamous cell carcinomas of the lung, cervix, ovaries, head and neck, oesophagus, and/or skin. In other embodiments, small molecule YAP/TAZ-TEAD inhibitors are useful for the treatment of cancers that originate from neuroectoderm-derived tissues, such as ependymomas, meningiomas, schwannomas, peripheral nerve-sheet tumors and/or neuroblastomas. In other embodiments, small molecule YAP/TAZ-TEAD inhibitors are useful for the treatment of vascular cancers, such as epithelioid haemangioendotheliomas, or for the treatment of supratentorial ependymomas or porocarcinomas. In some embodiments, the solid tumors have gain-of-function gene amplifications, gene fusions or activating mutations in the YAP1 or WWTR1 (TAZ) genes. In some embodiments the solid tumors have loss-of-function mutations or deletions in the NF2, LATS1/2, BAP1, FAT1, SAV1, and/or MST1/2 genes. In some embodiments solid tumors have gain-of-function mutations in the GNAQ and/or GNA11 genes, e.g. in uveal melanoma. In some embodiments, solid cancer are characterized by constitutive nuclear presence of YAP and/or TAZ. In some embodiments, solid cancers are characterized by the overexpression of YAP/TAZ-TEAD signature genes, including but not limited to CTGF, CYR61, AMOTL2, and/or ANKRD1.

Small molecule YAP/TAZ-TEAD inhibitors may also be useful to treat cancers that have developed resistance to prior treatments. This may include, for instance, the treatment of cancers that have developed resistance to chemotherapy, or to targeted therapy. In some embodiments, this may include the treatment of cancers that have developed resistance to inhibitors of receptor tyrosine kinases, such as EGFR (afatinib, erlotinib hydrochloride, osimertinib, gefitinib, dacomitinib, neratinib, canertinib, cetuximab) or AXL (crizotinib, cabozantinib, gilteritinib, sitravatinib, bemcentinib, dubermatinib), to components of the RAS-MAPK signaling cascade, including inhibitors of RAS itself (such as AMG510, MRTX849, B11701963, ARS1620), inhibitors of B-RAF (sorafinib tosylate, dabrafenib, vemurafenib, regorafenib), or MEK1/2 (trametinib, selumetinib, cobimetinib, mirdametinib).

Small molecule YAP/TAZ-TEAD inhibitors may also be useful when combined, upon simultaneous administration, or subsequent administration, with other agents used for the treatment of cancer. This may include, for instance, the co-treatment with inhibitors or monoclonal antibodies targeting receptor tyrosine kinases such as EGFR (afatinib, erlotinib hydrochloride, osimertinib, gefitinib, dacomitinib, neratinib, canertinib, cetuximab) or AXL (crizotinib, cabozantinib, gilteritinib, sitravatinib, bemcentinib, dubermatinib), to components of the RAS-MAPK signaling cascade, including inhibitors of RAS itself (such as AMG510, MRTX849, B11701963, ARS1620), inhibitors of B-RAF (sorafinib tosylate, dabrafenib, vemurafenib, regorafenib), or MEK1/2 (trametinib, selumetinib, cobimetinib, mirdametinib).

Small molecule YAP/TAZ-TEAD inhibitors may also be useful to treat a metastasized cancer. In some instances, the metastasized cancer is selected from metastasized uveal melanoma, mesothelioma, esophageal cancer, liver cancer, breast cancer, hepatocellular carcinoma, lung adenocarcinoma, glioma, colon cancer, colorectal cancer, gastric cancer, medulloblastoma, ovarian cancer, esophageal squamous cell carcinoma, sarcoma, Ewing sarcoma, head and neck cancer, prostate cancer, and meningioma.

In some embodiments, the cancer treated could be malignant pleural mesothelioma or lung cancer.

In some embodiments, the compounds of the disclosure can be used for the treatment of acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

### Malignant Pleural Mesothelioma

Small molecule YAP/TAZ-TEAD inhibitors may also be useful to treat malignant pleural mesothelioma, as a single agent, or in combination with inhibitors such as pemetrexed disodium, raltitrexed, carboplatin, oxaliplatin, gemcitabine, doxorubicin, or monoclonal antibodies such as bevacizumab. Combinations with checkpoint inhibitors such as pembrolizumab, atezolizumab, and/or nivolumab. Combinations with cell therapy, for instance, chimeric antigen receptor (CAR) T therapy or CAR NK therapy, which may, for instance, use mesothelin (MSLN) as an antigen. Combinations with monoclonal antibodies that, for instance, recognize mesothelin as an antigen, for instance BMS-986148, BAY 94-9343, amatuximab, and/or LMB-100.

### Lung Cancer

Small molecule YAP/TAZ-TEAD inhibitors may also be useful to treat lung cancer, as a single agent, or in combination with inhibitors such as afatinib, bevacizumab, cabozantinib, ceritinib, crizotinib, erlotinib hydrochloride, osimertinib, ramucirumab, gefitinib, alectinib, trastuzumab, cetuximab, ipilimumab, trametinib, dabrafenib, vemurafenib, dacomitinib, tivantinib, and/or onartuzumab. Combinations with checkpoint inhibitors such as pembrolizumab, atezolizumab, and/or nivolumab. Combinations with cisplatin, carboplatin, paclitaxel, paclitaxel protein bound, docetaxel, gemcitabine, vinorelbine, etoposide, nintedanib, vinblastine, pemetrexed, afatinib, bevacizumab, cabozantinib, ceritinib, crizotinib, erlotinib hydrochloride, osimertinib, ramucirumab, gefitinib, necitumumab, alectinib, trastuzumab, cetuximab, ipilimumab, trametinib, dabrafenib, vemurafenib, dacomitinib, tivantinib, onartuzumab, pembrolizumab, atezolizumab, and/or nivolumab

In some embodiments, small molecule YAP/TAZ-TEAD inhibitors are useful, e.g., for the treatment of congenital disorders. In some embodiments, the congenital disease is mediated by activation of transcriptional coactivator with PDZ binding motif/Yes—associated protein transcription coactivator (TAZ/YAP). In some embodiments, the congenital disease is characterized by a mutant Ga-protein. In some embodiments, the mutant Ga-protein is selected from G12, G13, Gq, G11, Gi, Go, and Gs. In some embodiments, the congenital disease is characterized by loss-of-function mutations or deletions in the NF2 gene. Exemplary congenital diseases include, but are not limited to, Sturge-Weber Syndrome, Port-Wine stain, and Neurofibromatosis. In some embodiments the congenital disease is Neurofibromatosis, including but not limited to Neurofibromatosis type 2.

In some embodiments, small molecule YAP/TAZ-TEAD inhibitors are useful, e.g., for the treatment of fibrotic disorders, such as fibrosis of the liver, the lung, the kidney, the heart or the skin. In some embodiments, fibrosis can be treated in the context of non-alcoholic fatty liver disease, primary sclerosing cholangitis, primary biliary cirrhosis, idiopathic pulmonary fibrosis, chronic kidney disease, and/or myocardial infarction injury.

The compounds of the disclosure can inhibit YAP/TAZ-TEAD transcription activation. The compounds have been shown to inhibit YAP/TAZ-TEAD transcription activity in cellular models and in an animal model. The compounds have also been shown to have an inhibitory effect on cancer cell lines that are dependent on YAP/TAZ-TEAD transcription activity and on the growth of cancer in a xenograft cancer model.

The compounds of the disclosure can optionally be bound covalently to an insoluble matrix and used for affinity chromatography (separations, depending on the nature of the groups of the compounds, for example compounds with pendant aryl are useful in hydrophobic affinity separations).

When using one or more derivatives of the formulae as defined herein:
the active ingredients of the compound(s) may be administered to the animal or mammal (including a human) to be treated by any means well known in the art, i.e. orally, intranasally, subcutaneously, intramuscularly, intradermally, intravenously, intra-arterially, parenterally or by catheterization.
the therapeutically effective amount of the preparation of the compound(s), especially for the treatment of diseases mediated by activity of YAP/TAZ-TEAD transcription in humans and other mammals (such as cancer, fibrosis and certain congenital disorders), preferably is a YAP/TAZ-TEAD transcription inhibiting amount of the compounds of the formulae, statements, aspects and embodiments as defined herein and corresponds to an amount which ensures a plasma level that is able to inhibit the YAP/TAZ-TEAD activation and is between 1 μg/ml and 100 mg/ml.

Suitable dosages of the compounds or compositions of the disclosure should be used to treat or prevent the targeted diseases in a subject. Depending upon the pathologic condition to be treated and the patient's condition, the said effective amount may be divided into several sub-units per day or may be administered at more than one day intervals.

According to a particular embodiment of the disclosure, the compounds of the invention may be employed in combination with other therapeutic agents for the treatment or prophylaxis of diseases mediated by activity of YAP/TAZ-TEAD transcription in humans and other mammals (such as cancer, fibrosis and certain congenital disorders). The disclosure therefore relates to the use of a composition comprising:
(a) one or more compounds of the formulae and aspects, statements and embodiments herein, and
(b) one or more further therapeutic or preventive agents that are used for the prevention or treatment of cancer or fibrosis as biologically active agents in the form of a combined preparation for simultaneous, separate or sequential use.

The compound or composition can be administered concurrently with, prior to, or subsequent to the one or more additional therapeutic agents, which are different from the compound described herein and may be useful as, e.g., combination therapies.

Examples of such further therapeutic agents for use in combinations include agents that are inhibitors of:
EGFR (such as afatinib, erlotinib hydrochloride, osimertinib, gefitinib, dacomitinib, neratinib, canertinib, cetuximab),
AXL (such as crizotinib, cabozantinib, gilteritinib, sitravatinib, bemcentinib, dubermatinib),
components of the RAS-MAPK signaling cascade, including inhibitors of RAS itself (such as AMG510, MRTX849, B11701963, ARS1620),
B-RAF (such as sorafinib tosylate, dabrafenib, vemurafenib, regorafenib), or
MEK1/2 (trametinib, selumetinib, cobimetinib, mirdametinib).

The pharmaceutical composition or combined preparation according to this disclosure may contain the compounds of the present disclosure over a broad content range depending on the contemplated use and the expected effect of the preparation. Generally, the content of the derivatives of the present disclosure of the combined preparation is within the range of 0.1 to 99.9% by weight, preferably from 1 to 99% by weight, more preferably from 5 to 95% by weight.

Those of skill in the art will also recognize that the compounds of the disclosure may exist in many different protonation states, depending on, among other things, the pH of their environment. While the structural formulae provided herein depict the compounds in only one of several possible protonation states, it will be understood that these structures are illustrative only, and that the disclosure is not limited to any particular protonation state—any and all protonated forms of the compounds are intended to fall within the scope of the disclosure.

The term “pharmaceutically acceptable salts” as used herein means the therapeutically active non-toxic salt forms which the compounds of formulae herein are able to form. Therefore, the compounds of this disclosure optionally comprise salts of the compounds herein, especially pharmaceutically acceptable non-toxic salts containing, for example, Na⁺, Li⁺, K⁺, Ca²⁺ and Mg²⁺. Such salts may include those derived by combination of appropriate cations such as alkali and alkaline earth metal ions or ammonium and quaternary amino ions with an acid anion moiety, typically a carboxylic acid. The compounds of the disclosure may bear multiple positive or negative charges. The net charge of the compounds of the disclosure may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. It will be understood that the identity of any associated counter ion is not a critical feature of the disclosure, and that the disclosure encompasses the compounds in association with any type of counter ion. Moreover, as the compounds can exist in a variety of different forms, the disclosure is intended to encompass not only forms of the compounds that are in association with counter ions (e.g., dry salts), but also forms that are not in association with counter ions (e.g., aqueous or organic solutions). Metal salts typically are prepared by reacting the metal hydroxide with a compound of this disclosure. Examples of metal salts which are prepared in this way are salts containing Li⁺, Na⁺, and K⁺. A less soluble metal salt can be precipitated from the solution of a more soluble salt by addition of the suitable metal compound. In addition, salts may be formed from acid addition of certain organic and inorganic acids to basic centers, typically amines, or to acidic groups.

Examples of such appropriate acids include, for instance, inorganic acids such as hydrohalogen acids, e.g. hydrochloric or hydrobromic acid, sulfuric acid, nitric acid, phosphoric acid and the like; or organic acids such as, for example, acetic, propanoic, hydroxyacetic, 2-hydroxypropanoic, 2-oxopropanoic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclohexanesulfamic, salicylic (i.e. 2-hydroxybenzoic), p-aminosalicylic and the like. Furthermore, this term also includes the solvates which the compounds of formulae herein as well as their salts are able to form, such as for example hydrates, alcoholates and the like. Finally, it is to be understood that the compositions herein comprise compounds of the disclosure in their unionized, as well as zwitterionic form, and combinations with stoichiometric amounts of water as in hydrates.

Also included within the scope of this disclosure are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The compounds of the disclosure also include physiologically acceptable salts thereof. Examples of physiologically acceptable salts of the compounds of the disclosure include salts derived from an appropriate base, such as an alkali metal (for example, sodium), an alkaline earth (for example, magnesium), ammonium and NX₄_{⁺} (wherein X is C₁-C₄ alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound containing a hydroxy group include the anion of said compound in combination with a suitable cation such as Na⁺ and NX₄_{⁺} (wherein X typically is independently selected from H or a C₁-C₄ alkyl group). However, salts of acids or bases which are not physiologically acceptable may also find use, for example, in the preparation or purification of a physiologically acceptable compound. All salts, whether or not derived form a physiologically acceptable acid or base, are within the scope of the present disclosure.

As used herein and unless otherwise stated, the term “enantiomer” means each individual optically active form of a compound of the disclosure, having an optical purity or enantiomeric excess (as determined by methods standard in the art) of at least 80% (e.g. at least 90% of one enantiomer and at most 10% of the other enantiomer), preferably at least 90% and more preferably at least 98%.

The term “isomers” as used herein means all possible isomeric forms, including tautomeric and stereochemical forms, which the compounds of formulae herein may possess, but not including position isomers. Typically, the structures shown herein exemplify only one tautomeric or resonance form of the compounds, but the corresponding alternative configurations are contemplated as well. Unless otherwise stated, the chemical designation of compounds denotes the mixture of all possible stereochemically isomeric forms, said mixtures containing all diastereomers and enantiomers (since the compounds of formulae herein may have at least one chiral center) of the basic molecular structure, as well as the stereochemically pure or enriched compounds. More particularly, stereogenic centers may have either the R- or S-configuration, and multiple bonds may have either cis- or trans-configuration.

Pure isomeric forms of the said compounds are defined as isomers substantially free of other enantiomeric or diastereomeric forms of the same basic molecular structure. In particular, the term “stereoisomerically pure” or “chirally pure” relates to compounds having a stereoisomeric excess of at least about 80% (e.g. at least 90% of one isomer and at most 10% of the other possible isomers), preferably at least 90%, more preferably at least 94% and most preferably at least 97%. The terms “enantiomerically pure” and “diastereomerically pure” should be understood in a similar way, having regard to the enantiomeric excess, respectively the diastereomeric excess, of the mixture in question.

Separation of stereoisomers is accomplished by standard methods known to those in the art. One enantiomer of a compound of the invention can be separated substantially free of its opposing enantiomer by a method such as formation of diastereomers using optically active resolving agents (“Stereochemistry of Carbon Compounds,” (1962) by E. L. Eliel, McGraw Hill; Lochmuller, C. H., (1975) J. Chromatogr., 113:(3) 283-302). Separation of isomers in a mixture can be accomplished by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers, or (3) enantiomers can be separated directly under chiral conditions. Under method (1), diastereomeric salts can be formed by reaction of enantiomerically pure chiral bases such as brucine, quinine, ephedrine, strychnine, a-methyl-b-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts. Alternatively, by method (2), the substrate to be resolved may be reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched compound. A method of determining optical purity involves making chiral esters, such as a menthyl ester or Mosher ester, a-methoxy-a-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (Hoye, T., WO 96/15111). Under method (3), a racemic mixture of two asymmetric enantiomers is separated by chromatography using a chiral stationary phase. Suitable chiral stationary phases are, for example, polysaccharides, in particular cellulose or amylose derivatives. Commercially available polysaccharide based chiral stationary phases are ChiralCel™ CA, OA, OB5, OC5, OD, OF, OG, OJ and OK, and Chiralpak™ AD, AS, OP(+) and OT(+). Appropriate eluents or mobile phases for use in combination with said polysaccharide chiral stationary phases are hexane and the like, modified with an alcohol such as ethanol, isopropanol and the like. (“Chiral Liquid Chromatography” (1989) W. J. Lough, Ed. Chapman and Hall, New York; Okamoto, (1990) “Optical resolution of dihydropyridine enantiomers by High-performance liquid chromatography using phenylcarbamates of polysaccharides as a chiral stationary phase”, J. of Chromatogr. 513:375-378).

The terms cis and trans are used herein in accordance with Chemical Abstracts nomenclature and include reference to the position of the substituents on a ring moiety. The absolute stereochemical configuration of the compounds of the formulae described herein may easily be determined by those skilled in the art while using well-known methods such as, for example, X-ray diffraction.

The present disclosure also includes isotopically labelled compounds, which are identical to those recited in the formulas recited herein, but for the fact that one or more atoms are replaced by an atom having an atomic mass or mass number different from the atomic mass or mass number usually found in nature. Examples of isotopes that may be incorporated into compounds of the present disclosure include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present disclosure and pharmaceutically acceptable salts of said compounds or which contain the aforementioned isotopes and/or other isotopes of other atoms are within the scope of this invention. Certain isotopically labeled compounds of the present disclosure, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be preferred in some circumstances. Isotopically labelled compounds of the formulas of this disclosure may generally be prepared by carrying out the procedures disclosed in the examples and preparations described herein, by substituting a readily available isotopically labelled reagent for a non-isotopically labelled reagent.

Also encompassed within the disclosure are modifications of the compounds of formula (I) or other formulas, embodiments, aspects or parts thereof or metabolites thereof using PROTAC technology (Schapira M. et al, Nat. Rev. Drug Discov. 2019, 18(12), 949-963). Specifically, the PROTAC technology designs a bifunctional small molecule, one end of which is a compound of the general formula (I) or other formulas, embodiments, aspects or parts thereof or metabolites thereof, and the other end of which is connected with a ligand of E3 ubiquitin ligase through a connecting chain, to form a target-induced protein degradation complex. Because this degradation has a catalytic effect, a lower dosage can achieve efficient degradation. The compound of the general formula (I) or other formulas, embodiments, aspects or parts thereof or metabolites thereof can be connected via a linker arm (e.g. long-chain ethylene glycol with the length of 2-10, long-chain propylene glycol with the length of 2-10 and long-chain fatty alkane with the length of 2-10) to a ligand of E3 ubiquitin ligase such as e.g. thalidomide analogs.

The compounds of the disclosure may be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. Formulations optionally contain excipients such as those set forth in the “Handbook of Pharmaceutical Excipients” (1986) and include ascorbic acid and other antioxidants, chelating agents such as EDTA, carbohydrates such as dextrin, hydroxyalkylcellulose, hydroxyalkylmethylcellulose, stearic acid and the like.

Subsequently, the term “pharmaceutically acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, e.g. the compositions of this disclosure can suitably be used as concentrates, emulsions, solutions, granulates, dusts, sprays, aerosols, suspensions, ointments, creams, tablets, pellets or powders.

Suitable pharmaceutical carriers for use in the said pharmaceutical compositions and their formulation are well known to those skilled in the art, and there is no particular restriction to their selection within the present disclosure. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, e.g. carriers and additives which do not create permanent damage to mammals. The pharmaceutical compositions of the present disclosure may be prepared in any known manner, for instance by homogeneously mixing, coating and/or grinding the active ingredients, in a one-step or multi-steps procedure, with the selected carrier material and, where appropriate, the other additives such as surface-active agents. may also be prepared by micronisation, for instance in view to obtain them in the form of microspheres usually having a diameter of about 1 to 10 gm, namely for the manufacture of microcapsules for controlled or sustained release of the active ingredients.

Suitable surface-active agents, also known as emulgent or emulsifier, to be used in the pharmaceutical compositions of the present disclosure are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable from coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl group having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcoholamine salts of dodecylbenzene sulphonic acid or dibutyl-naphthalenesulphonic acid or a naphthalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidyl-choline, dipalmitoylphoshatidyl-choline and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, particularly halides, having 4 hydrocarbon groups optionally substituted with halo, phenyl, substituted phenyl or hydroxy; for instance quaternary ammonium salts containing as N-substituent at least one C₈₋₂₂alkyl (e.g. cetyl, lauryl, palmityl, myristyl, oleyl and the like) and, as further substituents, unsubstituted or halogenated lower alkyl, benzyl and/or hydroxy-lower alkyl.

A more detailed description of surface-active agents suitable for this purpose may be found for instance in “McCutcheon's Detergents and Emulsifiers Annual” (MC Publishing Crop., Ridgewood, New Jersey, 1981), “Tensid-Taschenbucw’, 2 d ed. (Hanser Verlag, Vienna, 1981) and “Encyclopaedia of Surfactants, (Chemical Publishing Co., New York, 1981).

Compounds of the disclosure and their pharmaceutically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient.

While it is possible for the active ingredients to be administered alone it is preferable to present them as pharmaceutical formulations. The formulations, both for veterinary and for human use, of the present disclosure comprise at least one active ingredient, as above described, together with one or more pharmaceutically acceptable carriers therefore and optionally other therapeutic ingredients. The carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present disclosure suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein. For infections of the eye or other external tissues e.g. mouth and skin, the formulations are optionally applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include, for example, at least 30% w/w of a polyhydric alcohol, e.g. an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this disclosure may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The choice of suitable oils or fats for the formulation is based on achieving the desired cosmetic properties, since the solubility of the active compound in most oils likely to be used in pharmaceutical emulsion formulations is very low. Thus the cream should optionally be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. The active ingredient is optionally present in such formulations in a concentration of 0.5 to 20%, advantageously 0.5 to 10% particularly about 1.5% w/w. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc), which is administered in the manner in which snuff is taken, e.g. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this disclosure may include other agents conventional in the art having regard to the type of formulation in question, for example those suitable for oral administration may include flavoring agents.

Compounds of the disclosure can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given compound. Controlled release formulations adapted for oral administration in which discrete units comprising one or more compounds of the disclosure can be prepared according to conventional methods.

Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethylcellulose, polymethyl methacrylate and the other above-described polymers. Such methods include colloid drug delivery systems like liposomes, microspheres, microemulsions, nanoparticles, nanocapsules and so on. Depending on the route of administration, the pharmaceutical composition may require protective coatings. Pharmaceutical forms suitable for injectionable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof.

In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g. one of them may be in the form of an oral or parenteral formulation whereas the other is in the form of an ampoule for intravenous injection or an aerosol.

Another embodiment of this disclosure relates to various precursor or “pro-drug” forms of the compounds of the present disclosure. It may be desirable to formulate the compounds of the present disclosure in the form of a chemical species which itself is not significantly biologically-active, but which when delivered to the animal, mammal or human will undergo a chemical reaction catalyzed by the normal function of the body of the animal, mammal or human, inter alia, enzymes present in the stomach or in blood serum, said chemical reaction having the effect of releasing a compound as defined herein. The term “pro-drug” thus relates to these species which are converted in vivo into the active pharmaceutical ingredient.

The pro-drugs of the compounds of the present disclosure can have any form suitable to the formulator, for example, esters are non-limiting common pro-drug forms. In the present case, however, the pro-drug may necessarily exist in a form wherein a covalent bond is cleaved by the action of an enzyme present at the target locus. For example, a C—C covalent bond may be selectively cleaved by one or more enzymes at said target locus and, therefore, a pro-drug in a form other than an easily hydrolysable precursor, inter alia an ester, an amide, and the like, may be used. The counterpart of the active pharmaceutical ingredient in the pro-drug can have different structures such as an amino acid or peptide structure, alkyl chains, sugar moieties and others as known in the art.

For the purpose of the present disclosure, the term “therapeutically suitable pro-drug” is defined herein as “a compound modified in such a way as to be transformed in vivo to the therapeutically active form, whether by way of a single or by multiple biological transformations, when in contact with the tissues of the animal, mammal or human to which the pro-drug has been administered, and without undue toxicity, irritation, or allergic response, and achieving the intended therapeutic outcome”.

More specifically the term “prodrug”, as used herein, relates to an inactive or significantly less active derivative of a compound such as represented by the structural formulae herein described, which undergoes spontaneous or enzymatic transformation within the body in order to release the pharmacologically active form of the compound. For a comprehensive review, reference is made to Rautio J. et al. (“Prodrugs: design and clinical applications” Nature Reviews Drug Discovery, 2008, doi: 10.1038/nrd2468).

The compounds of the disclosure can be prepared while using a series of chemical reactions well known to those skilled in the art, altogether making up the process for preparing said compounds and exemplified further. The processes described further are only meant as examples and by no means are meant to limit the scope of the present disclosure.

The present disclosure relates to methods for the preparation of the compounds, comprising the steps of:
Cyclocondensation of 2-halomethyl anilines or 2-formyl anilines with suitable reagents to obtain 3-amino-3,4-dihydroquinolin-2(1H)-ones; or cyclocondensation of 2-formyl anilines followed by reduction to obtain (1,2,3,4-tetrahydroquinolin-3-yl)methanamines;
Reacting the previously obtained 3-amino-3,4-dihydroquinolin-2(1H)-ones or (1,2,3,4-tetrahydroquinolin-3-yl)methanamines with aryl, heteroaryl halides, boronic acid or boronic esters and suitable metal catalyst (Cu or Pd) to obtain 3-amino-1-(hetero)aryl-3,4-dihydroquinolin-2(1H)-ones or (1-(hetero)aryl-1,2,3,4-tetrahydroquinolin-3-yl)methanamines, respectively;
Further derivatization (e.g. acylation, sulfonylation and/or alkylation) of the amine moiety of 1-(hetero)aryl-1,2,3,4-tetrahydroquinolin-3-amines, which are obtained after reduction of previously obtained 3-amino-1-(hetero)aryl-3,4-dihydroquinolin-2(1H)-ones, or (1-(hetero)aryl-1,2,3,4-tetrahydroquinolin-3-yl)methanamines to provide the desired compounds of the invention.

The compounds of the present disclosure may be prepared according to the general procedure outlined in Scheme 1.

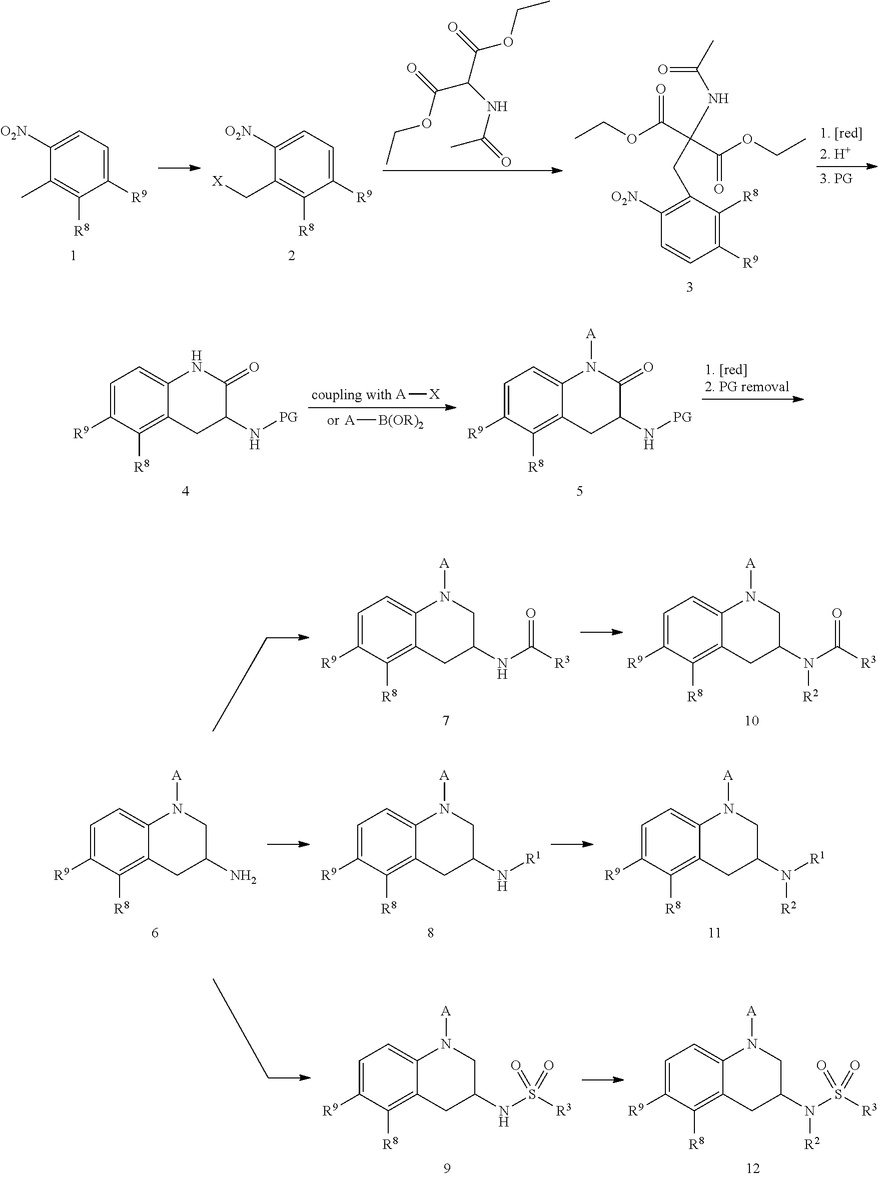

Scheme 1: all R¹, R², R³, R^{3a}, R⁸, R^{g}, A are as described for the compounds of the present invention and its embodiments and formulae. X=Halogen; PG=protecting group.

Substituted 1-(halomethyl)-2-nitrobenzenes of general formula 2 (X═Cl, Br), commercially available or synthesized from 1-methyl-2-nitrobenzene 1 by procedures known to the skilled in the art, may be reacted with diethyl 2-acetamidomalonate in the presence of a base (e.g. KOH, NaOEt, NaH, K₂CO₃ and the like) in a suitable solvent (e.g. MeOH, EtOH, DMF and the like) at a temperature raising from 50° C. to 100° C. to provide intermediates of general formula 3. Bicycles of general formula 4 may be obtained by reduction of the nitro moiety of intermediates of general formula 3 in a suitable solvent (e.g. MeOH, EtOH and the like) at a temperature raising from 50° C. to 100° C. followed by refluxing in concentrated acid (e.g. HCl and the like). More information can be found in WO2010/116270. Introduction of the N-protecting group to provide intermediates of general formula 4 may be performed following procedures known to the skilled in the art (e.g. PG=Boc or PMB). More information can be found in T. W. Greene and P. G. M. Wuts in Protective Groups in Organic Chemistry, 3^{rd} ed., John Wiley and Sons, 1999. Intermediates of general formula 4 may be reacted further with appropriate coupling agents selected from, but not limited to, halo(hetero)aryls, boronic acids, boronic esters, in combination with corresponding Pd or Cu catalysts to afford compounds of general formula 5. More information can be found in Synlett 2008, 614 and Organic and Biomolecular Chemistry 2017, 9288. Intermediates of general formula 6 may be obtained by reduction of intermediates of general formula 5 (e.g. treatment with BH₃·THF, or NaBH₄ and the like) at room temperature followed by removal of the N-protecting group via procedures known to the skilled in the art (e.g. treatment with a an acid (e.g. HCl, TFA and the like) if PG=Boc or by hydrogenation if PG=PMB). Compounds of interest having a general formula 7 may be obtained from amine derivatives of general formula 6 by reaction with acyl chlorides in presence of a base (e.g. TEA, DIPEA and the like) in an aprotic solvent (e.g. CH₂Cl₂, 1,4-dioxane and the like). Alternatively, amine derivatives of general formula 6 may be coupled with carboxylic acid derivatives under standard peptide coupling conditions in the presence of a coupling agent (e.g. T3P, HATU, EDC·HCl and the like) and a base (e.g. TEA, DIPEA and the like) in a polar aprotic solvent (e.g. CH₂Cl₂, DMF and the like) to provide the amides of general formula 7. Amine derivatives of general formula 8 may be obtained from amine derivatives of general formula 6 by reaction with alkyl halides or electron poor, polarized double bonds (Michael acceptors; Tetrahedron 2019, 2371) in the presence of a base (e.g. K₂CO₃, TEA, DIPEA and the like) in a suitable solvent (e.g. CH₂Cl₂, ACN and the like). Sulfonamide derivatives of general formula 9 may be obtained from amine derivatives of general formula 6 by reaction with sulfonyl chlorides in the presence of a base (e.g. TEA, DIPEA and the like) in a suitable solvent (e.g. CH₂Cl₂, ACN and the like). Secondary amides of general formulae 10, 11, and 12 may be obtained from amine derivatives of general formula 7, 8, and 9, respectively, by reaction with alkyl halides in the presence of a base (e.g. NaH and the like) in a polar aprotic solvent (e.g. DMF and the like).

In another embodiment, compounds of the present disclosure may also be synthesized according to the general procedure outlined in Scheme 2.

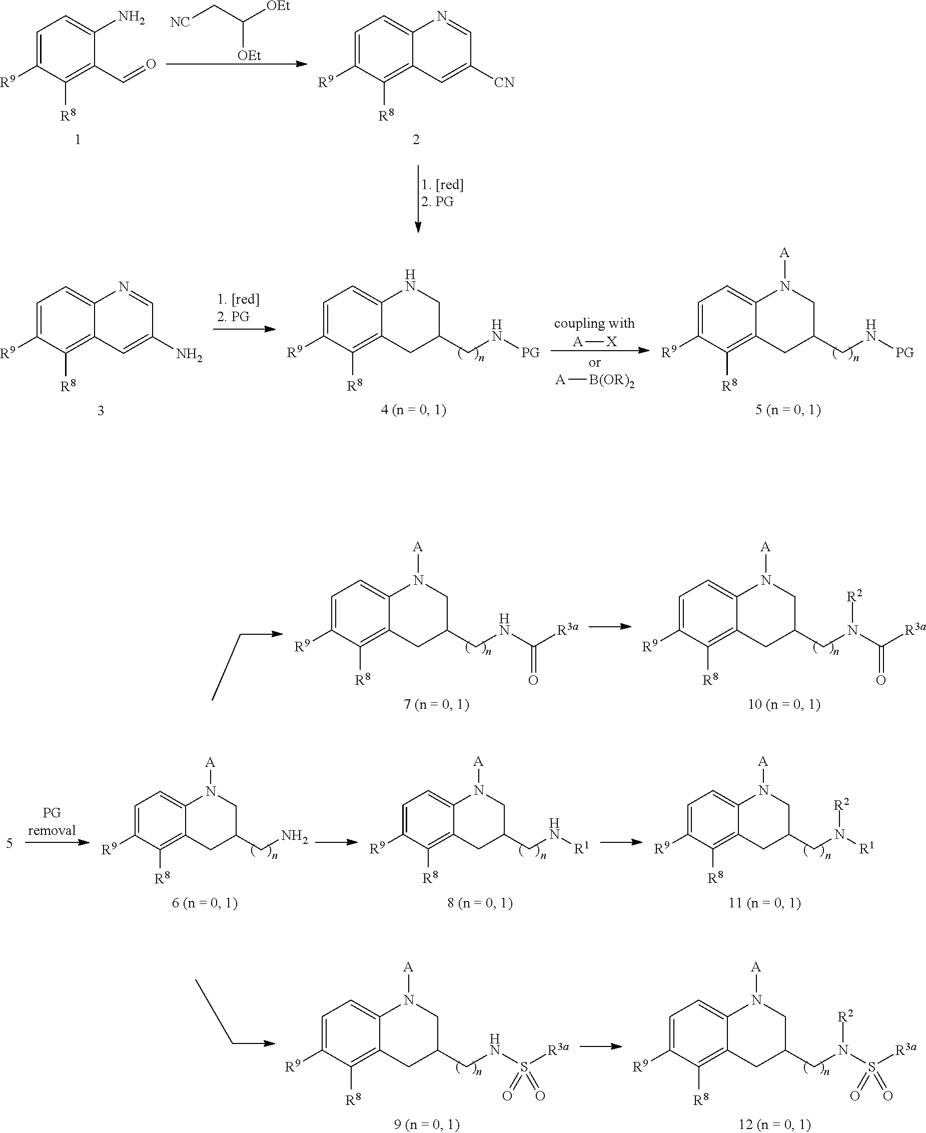

Scheme 2: all R¹, R², R³, R^{3a}, R⁸, R⁹, A are as described for the compounds of the present invention and its embodiments and formulae. X=Halogen; PG=protecting group.

Substituted 2-aminobenzaldehydes of general formula 1, commercially available or synthesized by procedures known to the skilled in the art, may be condensed with 3,3-diethoxypropanenitrile in the presence of an acid (e.g. H₂SO₄, pTSA and the like) in a suitable solvent (e.g. toluene and the like) at a temperature raising to 140° C. to provide intermediates of general formula 2. Bicycles of general formula 4 may be obtained from quinolin-3-amines of general formula 3 or quinoline-3-carbonitriles of general formula 2 through catalytic hydrogenation employing Raney Nickel in a suitable solvent (e.g. EtOH, THF and the like) at a temperature raising to 100° C., followed by the introduction of the N-protecting group via procedures known to the skilled in the art (e.g. PG=Boc or PMB). More information can be found in Bioorganic Medicinal Chemistry Letters 2005, 1895. Intermediates of general formula 4 may be reacted further with appropriate coupling agents selected from, but not limited to, halo(hetero)aryls, boronic acids, boronic esters, in combination with corresponding Pd or Cu catalysts to afford compounds of general formula 5. Intermediates of general formula 6 may be obtained by removal of the N-protecting group via procedures known to the skilled in the art (e.g. treatment with a an acid (e.g. HCl, TFA and the like) if PG=Boc or by hydrogenation if PG=PMB). Compounds of interest having the general formulae 7, 8, 9, 10, 11, and 12 may be obtained from intermediates of general formula 6 via procedures as described in Scheme 1.

Alternatively, compounds of the present disclosure may also be synthesized according to the general procedure outlined in Scheme 3.

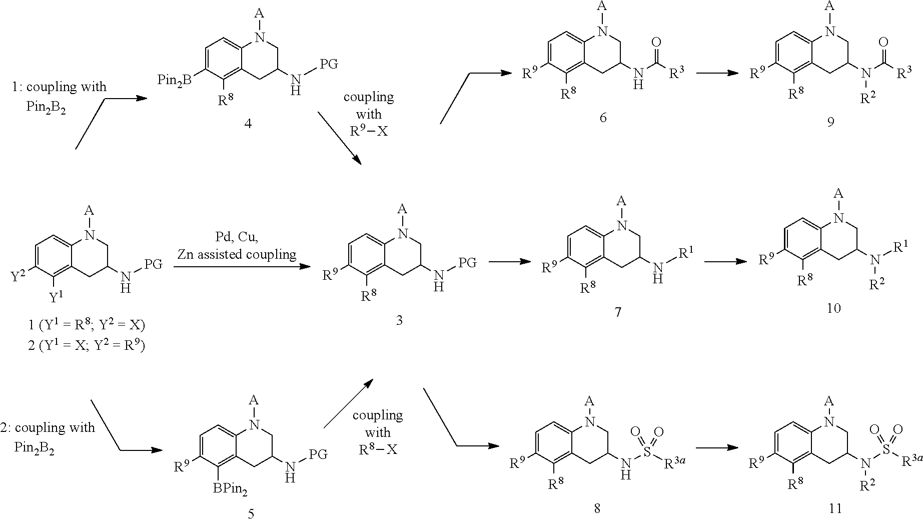

Scheme 3: all R¹, R², R³, R^{3a}, R⁸, R⁹, A are as described for the compounds of the present invention and its embodiments and formulae. X=Halogen; Y^{1,2}═R⁸, R⁹, or X; PG=protecting group.

Derivatives of general formula 1 and 2 wherein PG is a protecting group (e.g. PG=Boc or PMB), synthesized as described in Scheme 1, may be reacted with appropriate coupling agents selected from, but not limited to, boronic acids, boronic esters, anilines, alkyl amines, sodium alkoxides (European Journal of Organic Chemistry 2012, 4914), zinc cyanide (Journal of Medicinal Chemistry 2010, 3330), in combination with corresponding Pd or Cu catalysts to afford compounds of general formula 3. Boronate derivatives of general formula 4 and 5 may be obtained from intermediates of general formula 1 and 2, respectively, by reaction with bis(pinacolato)diboron in combination with corresponding Pd catalysts. Derivatives of general formula 4 and 5 may be reacted with coupling agents in combination with corresponding Pd catalysts to afford intermediate of general formula 3. Compounds of interest having the general formulae 6, 7, 8, 9, 10, and 11 may be obtained from intermediates of general formula 3 after removal of the N-protecting group via procedures known to the skilled in the art (e.g. treatment with a an acid (e.g. HCl, TFA and the like) if PG=Boc or by hydrogenation if PG=PMB) followed by procedures as described in Scheme 1.

Compounds of the present invention may also be synthesized according to the general procedure outlined in Scheme 4.

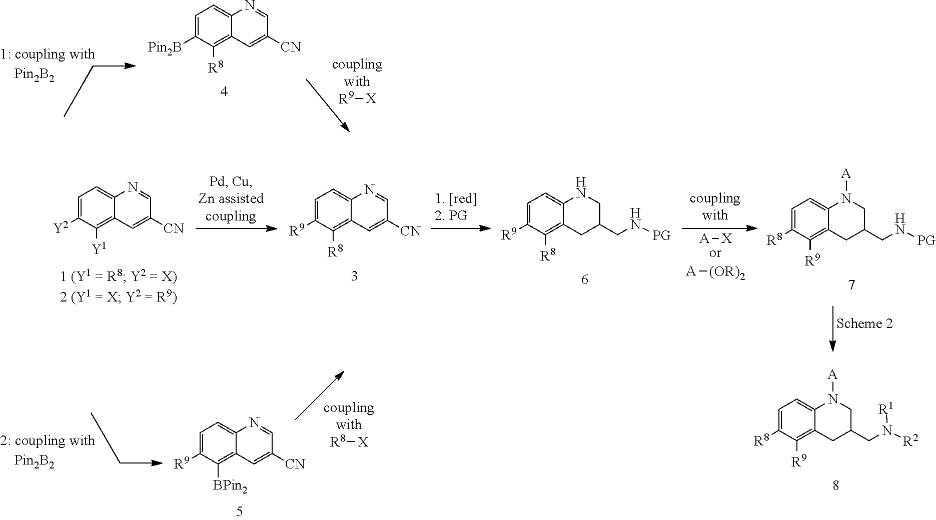

Scheme 4: all R¹, R², R⁸, R⁹, A are as described for the compounds of the present invention and its embodiments and formulae. X=Halogen; Y^{1,2}═R⁸, R⁹, or X; PG=protecting group.

Derivatives of general formula 1 and 2, commercially available or synthesized as described in Scheme 2, may be reacted with appropriate coupling agents selected from, but not limited to, boronic acids, boronic esters, anilines, alkyl amines, sodium alkoxides, zinc cyanide in combination with corresponding Pd or Cu catalysts to afford compounds of general formula 3. Boronate derivatives of general formula 4 and 5 may be obtained from intermediates of general formula 1 and 2, respectively, by reaction with bis(pinacolato)diboron in combination with corresponding Pd catalysts. Derivatives of general formula 4 and 5 may be reacted with coupling agents in combination with corresponding Pd catalysts to afford intermediate of general formula 3. Compounds of interest having the general formulae 8 may be obtained from intermediates of general formula 3 following the procedures as described in Scheme 2.

In another embodiment, compounds of the present disclosure may also be synthesized according to the general procedure outlined in Scheme 5.

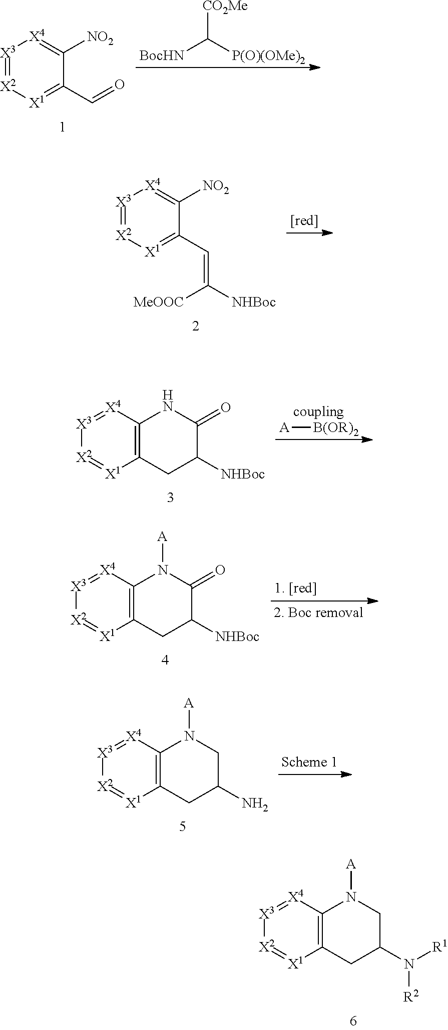

Scheme 5: all R¹, R², X¹, X², X³, X⁴, A are as described for the compounds of the present invention and its embodiments and formulae.

Nitro heteroaryl carbaldehydes of general formula 2, commercially available or synthesized by procedures known to the skilled in the art, may be condensed with methyl 2-((tert-butoxycarbonyl)amino)-2-(dimethoxyphosphoryl)acetate in the presence of a tetramethylguanidine in a suitable solvent (e.g. THF, 1,4-dioxane, and the like) at a temperature raising from −78° C. to room temperature to provide intermediates of general formula 2. More information can be found in WO2005/020987 and WO2006/059164. Bicycles of general formula 3 may be obtained by catalytic (Pd/C) hydrogenation of intermediates of general formula 2 in a suitable solvent (e.g. MeOH, EtOH and the like) at room temperature. Intermediates of general formula 3 may be reacted further with appropriate coupling agents selected from, but not limited to, halo(hetero)aryls, boronic acids, boronic esters, in combination with corresponding Pd or Cu catalysts to afford compounds of general formula 4. Intermediates of general formula 5 may be obtained by reduction of intermediates of general formula 4 (e.g. treatment with BH₃·THF, or NaBH₄ and the like) at room temperature followed by removal of the N-protecting group via procedures known to the skilled in the art (e.g. treatment with a an acid (e.g. HCl, TFA and the like)). Compounds of interest having a general formula 6 may be obtained from amine derivatives of general formula 5 by reactions as described in Scheme 1.

The general schemes depicted above should be considered as non-limiting examples. It will be understood that compounds of the invention may be obtained through other methods which are known to people skilled in the art.

Abbreviations used in the instant specification, particularly in the schemes and examples, are as follows: Ac—Acetyl, ACN—acetonitrile, AcCl—Acetyl chloride, AcOH—Acetic acid, Ac₂O-Acetic anhydride, aq.—Aqueous, AIBN—Azobisisobutyronitrile, BF₃·OEt₂—Boron trifluoride diethyl etherate complex, BH₃·THF—Borane tetrahydrofuran complex solution, BINAP—2,2′-Bis(diphenylphosphino)-1,1′-binaphthyl, BnBr—Benzyl bromide, Boc—tert-butyloxycarbonyl, (Boc)₂O—Di-tert-butyl dicarbonate, BzCl-Benzoyl chloride, Cs₂CO₃— Caesium carbonate, CDC₃ Deuterated chloroform, CHCl₃— Chloroform, Conc.—Concentrated, mCPBA—meta-Chloroperoxybenzoic acid, CuBr—Copper bromide, CuCN—Copper cyanide, Cul—Copper iodide, Cu(OAc)₂— Copper(II)acetate, CuTMEDA—Copper tetramethylethylenediamine, DCC—N,N′-Dicyclohexylcarbodiimide, DCM—Dichloromethane, DEA—Diethylamine, DIBAL-H-Diisobutylaluminium hydrid, DIPEA—N,N-Diisopropylethylamine, DMAP—4-Dimehtylaminopyridine, DMC—Dimethyl carbonate, DMF—N,N-Dimethylformamide, DMF-DMA —N,N-dimethylaminoformamide dimethyl acetale, DMP—Dess-Martin periodinane (3-oxo-1λ⁵-benzo[d][1,2]iodaoxole-1,1,1(3H)-triyl triacetate), DMSO-d₆—Deuterated dimethyl sulfoxide, EDC·HCl-1-Ethyl-3-(3-dimethylaminopropyl)carbodiimide hydrogen chloride, En—Enantiomer, Et₂O—Diethyl ether, EtOH—Ethanol, EtOAc—Ethyl acetate, Eq.—Equivalent, FA—Formic acid, Fmoc—Fluorenylmethyloxycarbonyl, Fmoc-Cl—Fluorenylmethyloxycarbonylchloride, h—Hour, HATU—O-(7-Azabenzotriazol-1-yl)—N,N,N′,N′-tetramethyluronium hexafluorophosphate, HCl-Hydrogen chloride, HNO₃— Nitric acid, HOBt—1-Hydroxybenzotriazole, HOSu—N-Hydroxysuccinimide, H₂SO₄— Sulphuric acid, K₂CO₃— Potassium carbonate, KF—Potassium fluoride, KI—Potassium iodide, KNO₃— Potassium nitrate, KOAc—Potassium acetate, KOtBu-Potassium tert-butoxide, K₃PO₄— Potassium phosphate, LAH—Lithiumaluminiumhydride, LiHMDS Lithium hexamethyldisilazide, LiOH·H₂O—Lithium hydroxide monohydrate, MeOH—methanol, min.—Minute, MsCI—Methanesulfonyl chloride, MW—Microwave radiation, NaBH₄— Sodium borohydride, NaBH₃CN—Sodium cyanoborohydride, NaN₃— Sodium azide, NaOtBu—Sodium tert-butoxide, NaOEt—Sodium ethoxide, NaH—Sodium hydride, NaOMe—Sodium methoxide, NaHCO₃— Sodium bicarbonate, NaIO₄— Sodium periodate, Na₂SO₄— Sodium sulfate, n-BuLi—n-ButylLithium, NBS—N-Bromosuccinimide, NH₃— Ammonia, NH₄OAc—Ammonium acetate, (NH₄)HCO₃— Ammonium bicarbonate, NH₄Cl—Ammonium chloride, NH₂Boc—tert-Butyl carbamate, NiCl₂·H₂O—Nickel(II) chloride hexahydrate, PBr₃— Phosphorus(III) bromide, Pd/C-Palladium on carbon, Pd₂(dba)₃—Tris(dibenzylideneacetone)dipalladium, Pd(dppf)Cl₂— (1,1′-Bis(diphenylphosphino)ferrocene)-dichloropalladium(II), Pd(dppf)Cl₂·DCM—(1,1′-Bis(diphenylphosphino)ferrocene)-dichloropalladium(II), complex with dichloromethane, Pd(OAc)₂— Palladium(II)acetate, Pd(PPh₃)₄—Tetrakis(triphenylphosphine)palladium(0), Pet ether Petroleum ether, Pic-BH₃— 2-Picoline-borane complex, Pin₂B2-Bis(pinacolato)diboron, PMBCI-4-Methoxybenzyl chloride, PtO₂— Platinum(IV) oxide, pTSA—p-Toluenesulfonic acid, PyBrop-Bromotripyrrolidinophosphonium hexafluorophosphate, Raney Ni—Raney nickel, RF—Retention factor, RT—Room temperature, sat.—Saturated, SFC—Supercritical fluid chromatography, SOCl₂—Thionyl chloride, TEA—Triethylamine, t-BuOH—tert-Butanol, THF—Tetrahydrofurane, TFA-Trifluoroacetic acid, TLC—thin layer chromatography, SPhos—Dicyclohexyl(2,6-dimethoxybiphenyl-2-yl)phosphine, XPhos-2-Dicyclohexylphosphino-2′,4′,6′-triisopropylbiphenyl, Xantphos-4,5-Bis(diphenylphosphino)-9,9-dimethylxanthene.

TABLE 1
Structures of example compounds of the disclosure and their respective codes

| Cpd No. | Structure | Name |
|---|---|---|
| 001 | 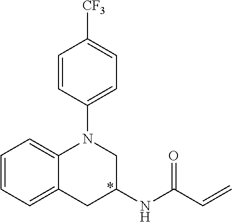 [SMILES: C=CC(=O)NC1Cc2ccccc2N(c2ccc(C(F)(F)F)cc2)C1] | N-(1-(4-(trifluoromethyl)- phenyl)-1,2,3,4-tetrahydro- quinolin-3-yl)acrylamide |
| 001- En1 | 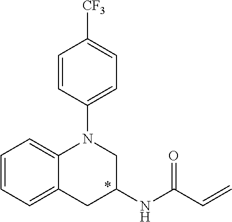 [SMILES: C=CC(=O)NC1Cc2ccccc2N(c2ccc(C(F)(F)F)cc2)C1] | (R)-or (S)-N-(1-(4- (trifluoromethyl)-phenyl)- 1,2,3,4-tetrahydro-quinolin- 3-yl)acrylamide |
| 001- En2 | 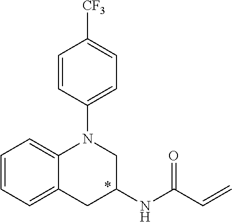 [SMILES: C=CC(=O)NC1Cc2ccccc2N(c2ccc(C(F)(F)F)cc2)C1] | (R)- or (S)-N-(1-(4- (trifluoromethyl)-phenyl)- 1,2,3,4-tetrahydro-quinolin- 3-yl)acrylamide |
| 002 | 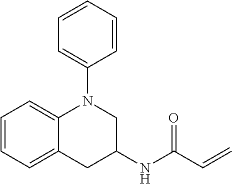 [SMILES: C=CC(=O)NC1Cc2ccccc2N(c2ccccc2)C1] | N-(1-phenyl-1,2,3,4- tetrahydroquinolin-3- yl)acrylamide |
| 003 | 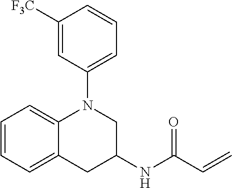 [SMILES: C=CC(=O)NC1Cc2ccccc2N(c2cccc(C(F)(F)F)c2)C1] | N-(1-(3-(trifluoromethyl)- phenyl)-1,2,3,4-tetrahydro- quinolin-3-yl)acrylamide |
| 004 | 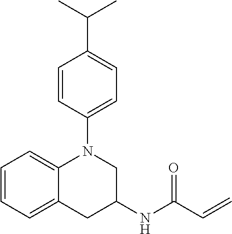 [SMILES: C=CC(=O)NC1Cc2ccccc2N(c2ccc(C(C)C)cc2)C1] | N-(1-(4-isopropylphenyl)- 1,2,3,4-tetrahydroquinolin- 3-yl)acrylamide |
| 005 | 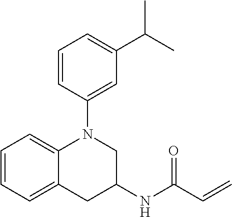 [SMILES: C=CC(=O)NC1Cc2ccccc2N(c2cccc(C(C)C)c2)C1] | N-(1-(3-isopropylphenyl)- 1,2,3,4-tetrahydroquinolin- 3-yl)acrylamide |
| 006 | 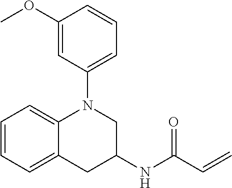 [SMILES: C=CC(=O)NC1Cc2ccccc2N(c2cccc(OC)c2)C1] | N-(1-(3-methoxyphenyl)- 1,2,3,4-tetrahydroquinolin- 3-yl)acrylamide |
| 007 | 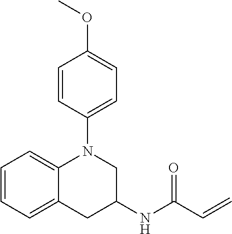 [SMILES: C=CC(=O)NC1Cc2ccccc2N(c2ccc(OC)cc2)C1] | N-(1-(4-methoxyphenyl)- 1,2,3,4-tetrahydroquinolin- 3-yl)acrylamide |
| 008 | 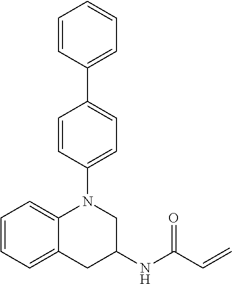 [SMILES: C=CC(=O)NC1Cc2ccccc2N(c2ccc(-c3ccccc3)cc2)C1] | N-(1-([1,1′-biphenyl]-4-yl)- 1,2,3,4-tetrahydroquinolin- 3-yl)acrylamide |
| 009 | 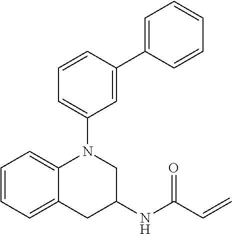 [SMILES: C=CC(=O)NC1Cc2ccccc2N(c2cccc(-c3ccccc3)c2)C1] | N-(1-([1,1′-biphenyl]-3-yl)- 1,2,3,4-tetrahydroquinolin- 3-yl)acrylamide |
| 010 | 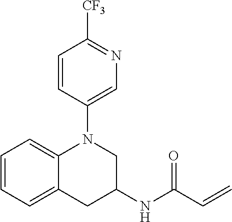 [SMILES: C=CC(=O)NC1Cc2ccccc2N(c2ccc(C(F)(F)F)nc2)C1] | N-(1-(6-(trifluoromethyl)- pyridin-3-yl)-1,2,3,4- tetrahydroquinolin-3- yl)acrylamide |
| 011 | 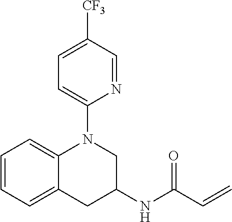 [SMILES: C=CC(=O)NC1Cc2ccccc2N(c2ccc(C(F)(F)F)cn2)C1] | N-(1-(5-(trifluoromethyl)- pyridin-2-yl)-1,2,3,4- tetrahydroquinolin-3- yl)acrylamide |
| 012 | 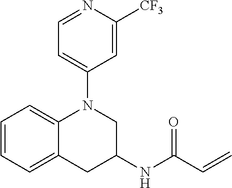 [SMILES: C=CC(=O)NC1Cc2ccccc2N(c2ccnc(C(F)(F)F)c2)C1] | N-(1-(2-(trifluoromethyl)- pyridin-4-yl)-1,2,3,4- tetrahydroquinolin-3- yl)acrylamide |
| 013 | 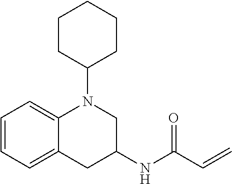 [SMILES: C=CC(=O)NC1Cc2ccccc2N(C2CCCCC2)C1] | N-(1-cyclohexyl-1,2,3,4- tetrahydroquinolin-3- yl)acrylamide |
| 014 | 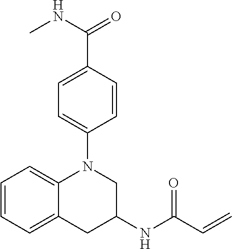 [SMILES: C=CC(=O)NC1Cc2ccccc2N(c2ccc(C(=O)NC)cc2)C1] | 4-(3-acrylamido-3,4- dihydroquinolin-1(2H)-yl)- N-methylbenzamide |
| 015 | 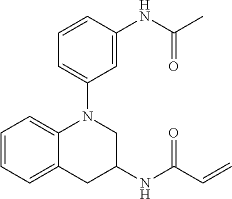 [SMILES: C=CC(=O)NC1Cc2ccccc2N(c2cccc(NC(C)=O)c2)C1] | N-(1-(3-acetamidophenyl)- 1,2,3,4-tetrahydroquinolin- 3-yl)acrylamide |
| 016 | 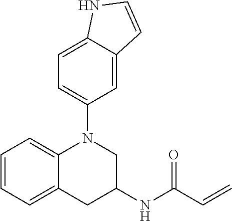 [SMILES: C=CC(=O)NC1Cc2ccccc2N(c2ccc3[nH]ccc3c2)C1] | N-(1-(1H-indol-5-yl)- 1,2,3,4-tetrahydroquinolin- 3-yl)acrylamide |
| 017 | 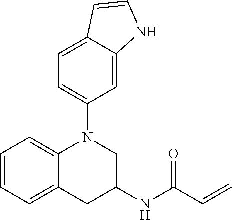 [SMILES: C=CC(=O)NC1Cc2ccccc2N(c2ccc3cc[nH]c3c2)C1] | N-(1-(1H-indol-6-yl)- 1,2,3,4-tetrahydroquinolin- 3-yl)acrylamide |
| 018 | 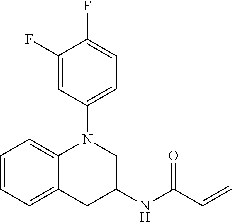 [SMILES: C=CC(=O)NC1Cc2ccccc2N(c2ccc(F)c(F)c2)C1] | N-(1-(3,4-difluorophenyl)- 1,2,3,4-tetrahydroquinolin- 3-yl)acrylamide |
| 019 | 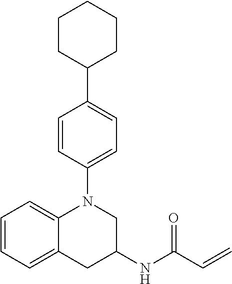 [SMILES: C=CC(=O)NC1Cc2ccccc2N(c2ccc(C3CCCCC3)cc2)C1] | N-(1-(4-cyclohexylphenyl)- 1,2,3,4-tetrahydroquinolin- 3-yl)acrylamide |
| 020 | 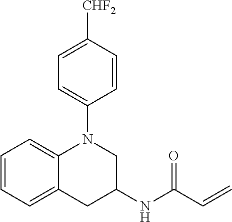 [SMILES: C=CC(=O)NC1Cc2ccccc2N(c2ccc(C(F)F)cc2)C1] | N-(1-(4-(difluoromethyl)- phenyl)-1,2,3,4-tetrahydro- quinolin-3-yl)acrylamide |
| 021 | 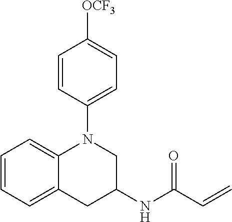 [SMILES: C=CC(=O)NC1Cc2ccccc2N(c2ccc(OC(F)(F)F)cc2)C1] | N-(1-(4-(trifluoromethoxy)- phenyl)-1,2,3,4-tetrahydro- quinolin-3-yl)acrylamide |
| 022 | 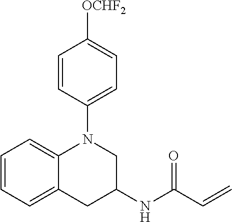 [SMILES: C=CC(=O)NC1Cc2ccccc2N(c2ccc(OC(F)F)cc2)C1] | N-(1-(4-(difluoromethoxy)- phenyl)-1,2,3,4-tetrahydro- quinolin-3-yl)acrylamide |
| 023 | 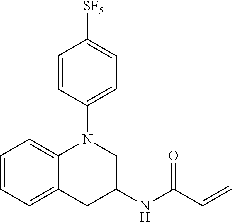 | N-(1-(4-(pentafluoro-λ⁶- sulfaneyl)phenyl)-1,2,3,4- tetrahydroquinolin-3- yl)acrylamide |
| 024 | 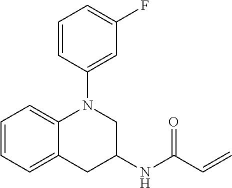 [SMILES: C=CC(=O)NC1Cc2ccccc2N(c2cccc(F)c2)C1] | N-(1-(3-fluorophenyl)- 1,2,3,4-tetrahydroquinolin- 3-yl)acrylamide |
| 025 | 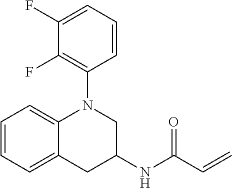 [SMILES: C=CC(=O)NC1Cc2ccccc2N(c2cccc(F)c2F)C1] | N-(1-(2,3-difluorophenyl)- 1,2,3,4-tetrahydroquinolin- 3-yl)acrylamide |
| 026 | 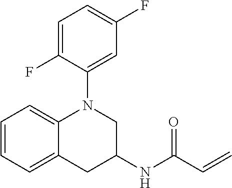 [SMILES: C=CC(=O)NC1Cc2ccccc2N(c2cc(F)ccc2F)C1] | N-(1-(2,5-difluorophenyl)- 1,2,3,4-tetrahydroquinolin- 3-yl)acrylamide |
| 027 | 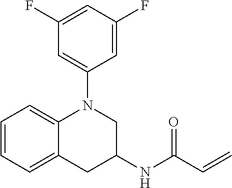 [SMILES: C=CC(=O)NC1Cc2ccccc2N(c2cc(F)cc(F)c2)C1] | N-(1-(3,5-difluorophenyl)- 1,2,3,4-tetrahydroquinolin- 3-yl)acrylamide |
| 028 | 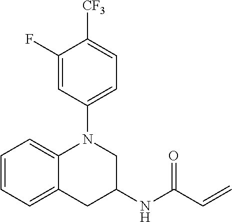 [SMILES: C=CC(=O)NC1Cc2ccccc2N(c2ccc(C(F)(F)F)c(F)c2)C1] | N-(1-(3-fluoro-4- (trifluoromethyl)phenyl)- 1,2,3,4-tetrahydroquinolin- 3-yl)acrylamide |
| 029 | 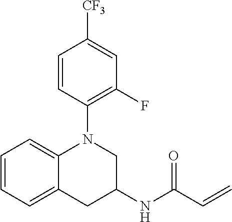 [SMILES: C=CC(=O)NC1Cc2ccccc2N(c2ccc(C(F)(F)F)cc2F)C1] | N-(1-(2-fluoro-4- (trifluoromethyl)phenyl)- 1,2,3,4-tetrahydroquinolin- 3-yl)acrylamide |
| 030 | 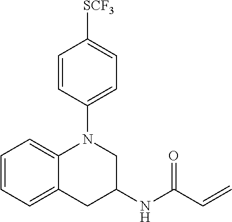 | N-(1-(4-((trifluoromethyl)- thio)phenyl)-1,2,3,4- tetrahydroquinolin-3- yl)acrylamide |
| 031 | 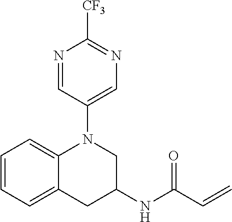 [SMILES: C=CC(=O)NC1Cc2ccccc2N(c2cnc(C(F)(F)F)nc2)C1] | N-(1-(2-(trifluoromethyl)- pyrimidin-5-yl)-1,2,3,4- tetrahydroquinolin-3- yl)acrylamide |
| 032 | 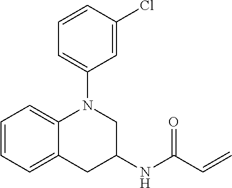 [SMILES: C=CC(=O)NC1Cc2ccccc2N(c2cccc(Cl)c2)C1] | N-(1-(3-chlorophenyl)- 1,2,3,4-tetrahydroquinolin- 3-yl)acrylamide |
| 033 | 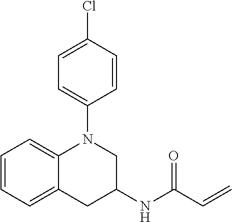 [SMILES: C=CC(=O)NC1Cc2ccccc2N(c2ccc(Cl)cc2)C1] | N-(1-(4-chlorophenyl)- 1,2,3,4-tetrahydroquinolin- 3-yl)acrylamide |
| 034 | 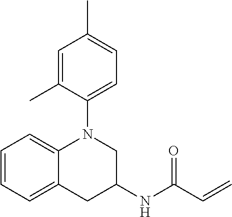 [SMILES: C=CC(=O)NC1Cc2ccccc2N(c2ccc(C)cc2C)C1] | N-(1-(2,4-dimethylphenyl)- 1,2,3,4-tetrahydroquinolin- 3-yl)acrylamide |
| 035 | 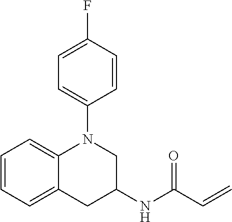 [SMILES: C=CC(=O)NC1Cc2ccccc2N(c2ccc(F)cc2)C1] | N-(1-(4-fluorophenyl)- 1,2,3,4-tetrahydroquinolin- 3-yl)acrylamide |
| 036 | 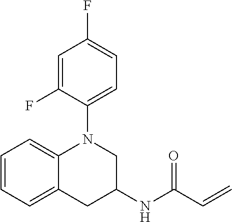 [SMILES: C=CC(=O)NC1Cc2ccccc2N(c2ccc(F)cc2F)C1] | N-(1-(2,4-difluorophenyl)- 1,2,3,4-tetrahydroquinolin- 3-yl)acrylamide |
| 037 | 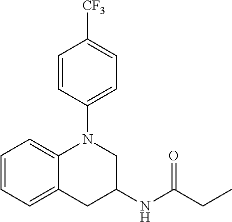 [SMILES: CCC(=O)NC1Cc2ccccc2N(c2ccc(C(F)(F)F)cc2)C1] | N-(1-(4-(trifluoromethyl)- phenyl)-1,2,3,4-tetrahydro- quinolin-3-yl)propionamide |
| 038 | 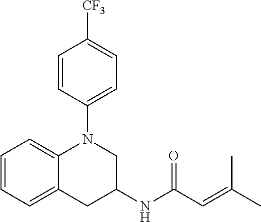 [SMILES: CC(C)=CC(=O)NC1Cc2ccccc2N(c2ccc(C(F)(F)F)cc2)C1] | 3-methyl-N-(1-(4- (trifluoromethyl)phenyl)- 1,2,3,4-tetrahydroquinolin- 3-yl)but-2-enamide |
| 039 | 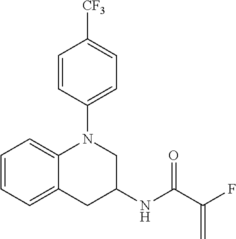 [SMILES: C=C(F)C(=O)NC1Cc2ccccc2N(c2ccc(C(F)(F)F)cc2)C1] | 2-fluoro-N-(1-(4- (trifluoromethyl)phenyl)- 1,2,3,4-tetrahydroquinolin- 3-yl)acrylamide |
| 040 | 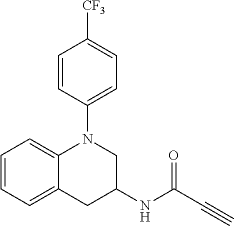 [SMILES: C#CC(=O)NC1Cc2ccccc2N(c2ccc(C(F)(F)F)cc2)C1] | N-(1-(4-(trifluoromethyl)- phenyl)-1,2,3,4-tetrahydro- quinolin-3-yl)propiolamide |
| 041 | 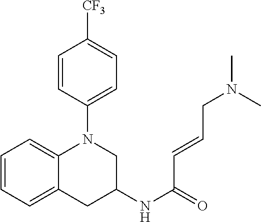 [SMILES: CN(C)C/C=C/C(=O)NC1Cc2ccccc2N(c2ccc(C(F)(F)F)cc2)C1] | (E)-4-(dimethylamino)-N- (1-(4-(trifluoromethyl)- phenyl)-1,2,3,4-tetrahydro- quinolin-3-yl)but-2-enamide |
| 042 | 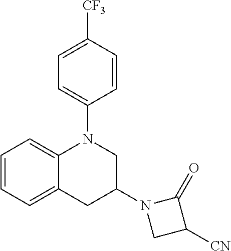 [SMILES: N#CC1CN(C2Cc3ccccc3N(c3ccc(C(F)(F)F)cc3)C2)C1=O] | 2-oxo-1-(1-(4- (trifluoromethyl)phenyl)- 1,2,3,4-tetrahydroquinolin- 3-yl)azetidine-3-carbonitrile |
| 043 | 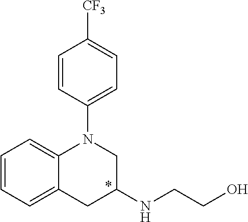 [SMILES: OCCNC1Cc2ccccc2N(c2ccc(C(F)(F)F)cc2)C1] | 2-((1-(4-(trifluoromethyl)- phenyl)-1,2,3,4-tetrahydro- quinolin-3-yl)amino)ethano- 1-ol |
| 043- En1 | 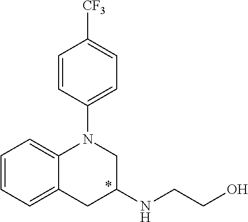 [SMILES: OCCNC1Cc2ccccc2N(c2ccc(C(F)(F)F)cc2)C1] | (R)- or (S)-2-((1-(4- (trifluoromethyl)-phenyl)- 1,2,3,4-tetrahydro-quinolin- 3-yl)amino)ethan-1-ol |
| 043- En2 | 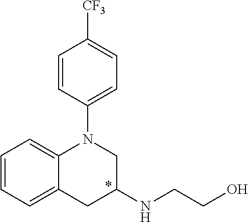 [SMILES: OCCNC1Cc2ccccc2N(c2ccc(C(F)(F)F)cc2)C1] | (R)- or (S)-2-((1-(4- (trifluoromethyl)-phenyl)- 1,2,3,4-tetrahydro-quinolin- 3-yl)amino)-ethan-1-ol |
| 044 | 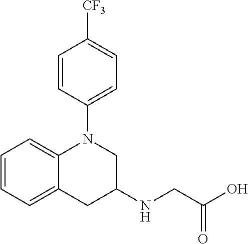 [SMILES: O=C(O)CNC1Cc2ccccc2N(c2ccc(C(F)(F)F)cc2)C1] | (1-(4-(trifluoromethyl)- phenyl)-1,2,3,4-tetrahydro- quinolin-3-yl)glycine |
| 045 | 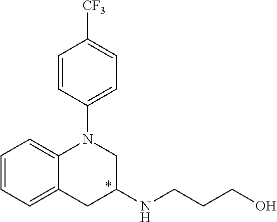 [SMILES: OCCCNC1Cc2ccccc2N(c2ccc(C(F)(F)F)cc2)C1] | 3-((1-(4-(trifluoromethyl)- phenyl)-1,2,3,4-tetrahydro- quinolin-3-yl)amino)- propan-1-ol |
| 045- En1 | 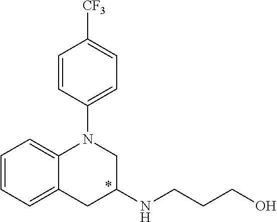 [SMILES: OCCCNC1Cc2ccccc2N(c2ccc(C(F)(F)F)cc2)C1] | (R)- or (S)-3-((1-(4- (trifluoromethyl)-phenyl)- 1,2,3,4-tetrahydro-quinolin- 3-yl)amino)-propan-1-ol |
| 045- En2 | 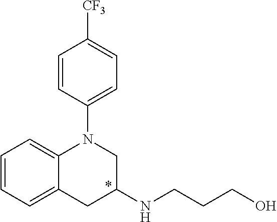 [SMILES: OCCCNC1Cc2ccccc2N(c2ccc(C(F)(F)F)cc2)C1] | (R)- or (S)-3-((1-(4- (trifluoromethyl)-phenyl)- 1,2,3,4-tetrahydro- quinolin- 3-yl)amino)-propan-1-ol |
| 046 | 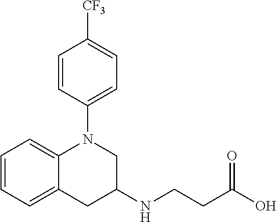 [SMILES: O=C(O)CCNC1Cc2ccccc2N(c2ccc(C(F)(F)F)cc2)C1] | 3-((1-(4-(trifluoromethyl)- phenyl)-1,2,3,4-tetrahydro- quinolin-3-yl)amino)- propanoic acid |
| 047 | 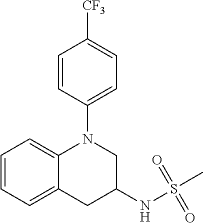 [SMILES: CS(=O)(=O)NC1Cc2ccccc2N(c2ccc(C(F)(F)F)cc2)C1] | N-(1-(4-(trifluoromethyl)- phenyl)-1,2,3,4-tetrahydro- quinolin-3-yl)methane- sulfonamide |
| 048 | 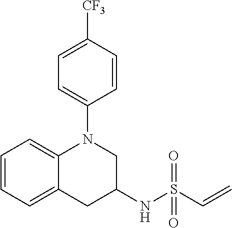 [SMILES: C=CS(=O)(=O)NC1Cc2ccccc2N(c2ccc(C(F)(F)F)cc2)C1] | N-(1-(4-(trifluoromethyl)- phenyl)-1,2,3,4-tetrahydro- quinolin-3-yl)ethane- sulfonamide |
| 049 | 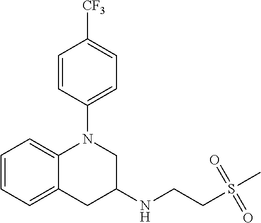 [SMILES: CS(=O)(=O)CCNC1Cc2ccccc2N(c2ccc(C(F)(F)F)cc2)C1] | N-(2-(methylsulfonyl)ethyl)- 1-(4-(trifluoromethyl)- phenyl)-1,2,3,4-tetrahydro- quinolin-3-amine |
| 050 | 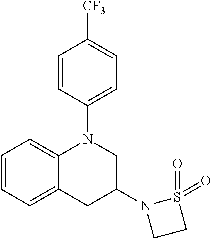 [SMILES: O=S1(=O)CCN1C1Cc2ccccc2N(c2ccc(C(F)(F)F)cc2)C1] | 2-(1-(4-(trifluoromethyl)- phenyl)-1,2,3,4-tetrahydro- quinolin-3-yl)-1,2- thiazetidine-1,1-dioxide |
| 051 | 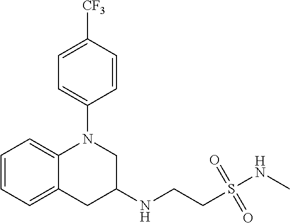 [SMILES: CNS(=O)(=O)CCNC1Cc2ccccc2N(c2ccc(C(F)(F)F)cc2)C1] | N-methyl-2-((1-(4- (trifluoromethyl)phenyl)- 1,2,3,4-tetrahydroquinolin- 3-yl)amino)ethane-1- sulfonamide |
| 052 | 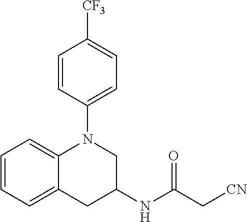 [SMILES: N#CCC(=O)NC1Cc2ccccc2N(c2ccc(C(F)(F)F)cc2)C1] | 2-cyano-N-(1-(4- (trifluoromethyl)phenyl)- 1,2,3,4-tetrahydroquinolin- 3-yl)acetamide |
| 053 | 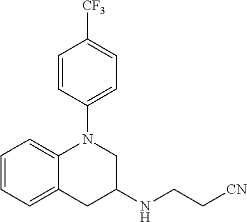 [SMILES: N#CCCNC1Cc2ccccc2N(c2ccc(C(F)(F)F)cc2)C1] | 3-((1-(4-(trifluoromethyl)- phenyl)-1,2,3,4- tetrahydroquinolin-3- yl)amino)propanenitrile |
| 054 | 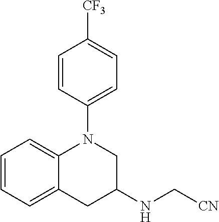 [SMILES: N#CCNC1Cc2ccccc2N(c2ccc(C(F)(F)F)cc2)C1] | 2-((1-(4-(trifluoromethyl)- phenyl)-1,2,3,4-tetrahydro- quinolin-3-yl)amino)- acetonitrile |
| 055 | 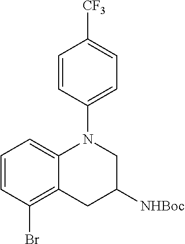 [SMILES: CC(C)(C)OC(=O)NC1Cc2c(Br)cccc2N(c2ccc(C(F)(F)F)cc2)C1] | tert-butyl (5-bromo-1-(4- (trifluoromethyl)phenyl)- 1,2,3,4-tetrahydroquinolin- 3-yl)carbamate |
| 056 | 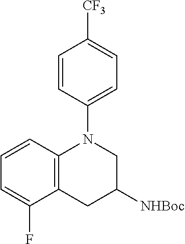 [SMILES: CC(C)(C)OC(=O)NC1Cc2c(F)cccc2N(c2ccc(C(F)(F)F)cc2)C1] | tert-butyl (5-fluoro-1-(4- (trifluoromethyl)phenyl)- 1,2,3,4-tetrahydroquinolin- 3-yl)carbamate |
| 057 | 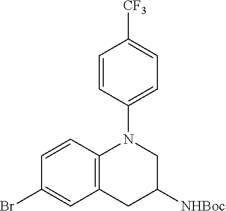 [SMILES: CC(C)(C)OC(=O)NC1Cc2cc(Br)ccc2N(c2ccc(C(F)(F)F)cc2)C1] | tert-butyl (6-bromo-1-(4- (trifluoromethyl)phenyl)- 1,2,3,4-tetrahydroquinolin- 3-yl)carbamate |
| 058 | 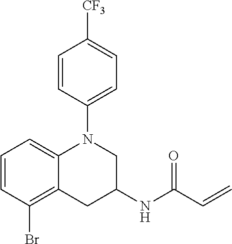 [SMILES: C=CC(=O)NC1Cc2c(Br)cccc2N(c2ccc(C(F)(F)F)cc2)C1] | N-(5-bromo-1-(4- (trifluoromethyl)phenyl)- 1,2,3,4-tetrahydroquinolin- 3-yl)acrylamide |
| 059 | 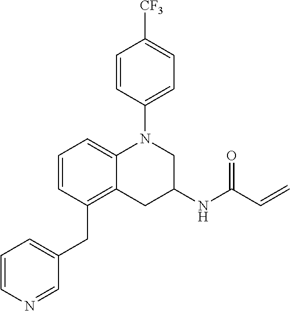 [SMILES: C=CC(=O)NC1Cc2c(Cc3cccnc3)cccc2N(c2ccc(C(F)(F)F)cc2)C1] | N-(5-(pyridin-3-ylmethyl)-1- (4-(trifluoromethyl)phenyl)- 1,2,3,4-tetrahydroquinolin- 3-yl)acrylamide |
| 060 | 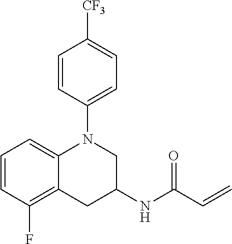 [SMILES: C=CC(=O)NC1Cc2c(F)cccc2N(c2ccc(C(F)(F)F)cc2)C1] | N-(5-fluoro-1-(4- (trifluoromethyl)phenyl)- 1,2,3,4-tetrahydroquinolin- 3-yl)acrylamide |
| 061 | 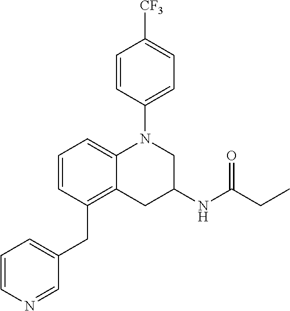 [SMILES: CCC(=O)NC1Cc2c(Cc3cccnc3)cccc2N(c2ccc(C(F)(F)F)cc2)C1] | N-(5-(pyridin-3-ylmethyl)-1- (4-(trifluoromethyl)phenyl)- 1,2,3,4-tetrahydroquinolin- 3-yl)propionamide |
| 062 | 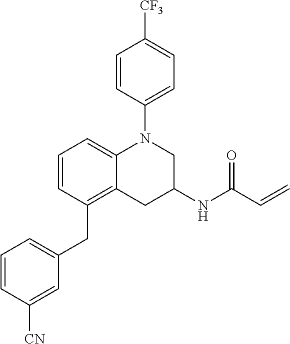 [SMILES: C=CC(=O)NC1Cc2c(Cc3cccc(C#N)c3)cccc2N(c2ccc(C(F)(F)F)cc2)C1] | N-(5-(3-cyanobenzyl)-1-(4- (trifluoromethyl)phenyl)- 1,2,3,4-tetrahydroquinolin- 3-yl)acrylamide |
| 063 | 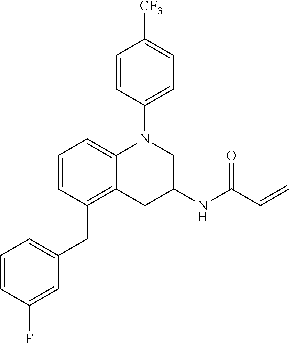 [SMILES: C=CC(=O)NC1Cc2c(Cc3cccc(F)c3)cccc2N(c2ccc(C(F)(F)F)cc2)C1] | N-(5-(3-fluorobenzyl)-1-(4- (trifluoromethyl)phenyl)- 1,2,3,4-tetrahydroquinolin- 3-yl)acrylamide |
| 064 | 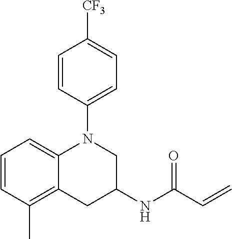 [SMILES: C=CC(=O)NC1Cc2c(C)cccc2N(c2ccc(C(F)(F)F)cc2)C1] | N-(5-methyl-1-(4- (trifluoromethyl)phenyl)- 1,2,3,4-tetrahydroquinolin- 3-yl)acrylamide |
| 065 | 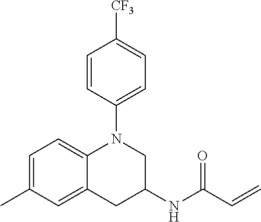 [SMILES: C=CC(=O)NC1Cc2cc(C)ccc2N(c2ccc(C(F)(F)F)cc2)C1] | N-(6-methyl-1-(4- (trifluoromethyl)phenyl)- 1,2,3,4-tetrahydroquinolin- 3-yl)acrylamide |
| 066 | 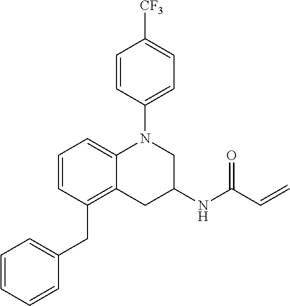 [SMILES: C=CC(=O)NC1Cc2c(Cc3ccccc3)cccc2N(c2ccc(C(F)(F)F)cc2)C1] | N-(5-benzyl-1-(4- (trifluoromethyl)phenyl)- 1,2,3,4-tetrahydroquinolin- 3-yl)acrylamide |
| 067 | 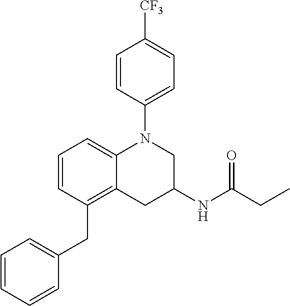 [SMILES: CCC(=O)NC1Cc2c(Cc3ccccc3)cccc2N(c2ccc(C(F)(F)F)cc2)C1] | N-(5-benzyl-1-(4- (trifluoromethyl)phenyl)- 1,2,3,4-tetrahydroquinolin- 3-yl)propionamide |
| 068 | 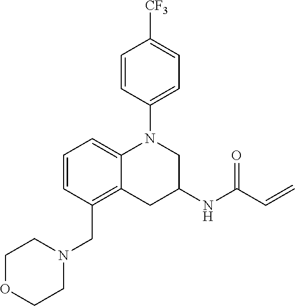 [SMILES: C=CC(=O)NC1Cc2c(CN3CCOCC3)cccc2N(c2ccc(C(F)(F)F)cc2)C1] | N-(5-(morpholinomethyl)-1- (4-(trifluoromethyl)- phenyl)-1,2,3,4- tetrahydroquinolin-3- yl)acrylamide |
| 069 | 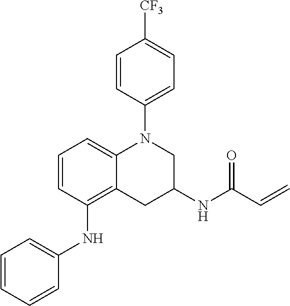 [SMILES: C=CC(=O)NC1Cc2c(Nc3ccccc3)cccc2N(c2ccc(C(F)(F)F)cc2)C1] | N-(5-(phenylamino)-1-(4- (trifluoromethyl)phenyl)- 1,2,3,4-tetrahydroquinolin- 3-yl)acrylamide |
| 070 | 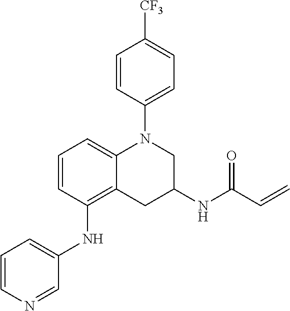 [SMILES: C=CC(=O)NC1Cc2c(Nc3cccnc3)cccc2N(c2ccc(C(F)(F)F)cc2)C1] | N-(5-(pyridin-3-ylamino)-1- (4-(trifluoromethyl)phenyl)- 1,2,3,4-tetrahydroquinolin- 3-yl)acrylamide |
| 071 | 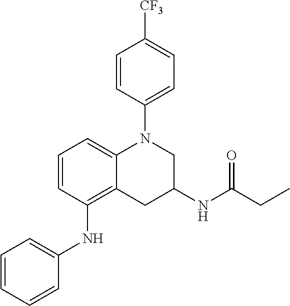 [SMILES: CCC(=O)NC1Cc2c(Nc3ccccc3)cccc2N(c2ccc(C(F)(F)F)cc2)C1] | N-(5-(phenylamino)-1-(4- (trifluoromethyl)phenyl)- 1,2,3,4-tetrahydroquinolin- 3-yl)propionamide |
| 072 | 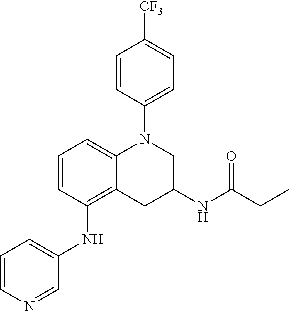 [SMILES: CCC(=O)NC1Cc2c(Nc3cccnc3)cccc2N(c2ccc(C(F)(F)F)cc2)C1] | N-(5-(pyridin-3-ylamino)-1- (4-(trifluoromethyl)phenyl)- 1,2,3,4-tetrahydroquinolin- 3-yl)propionamide |
| 073 | 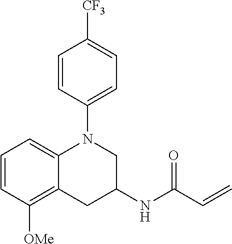 [SMILES: C=CC(=O)NC1Cc2c(OC)cccc2N(c2ccc(C(F)(F)F)cc2)C1] | N-(5-methoxy-1-(4- (trifluoromethyl)phenyl)- 1,2,3,4-tetrahydroquinolin- 3-yl)acrylamide |
| 074 | 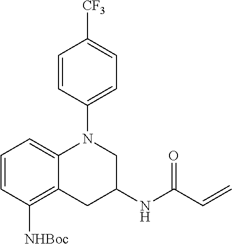 [SMILES: C=CC(=O)NC1Cc2c(NC(=O)OC(C)(C)C)cccc2N(c2ccc(C(F)(F)F)cc2)C1] | tert-butyl (3-acrylamido-1- (4-(trifluoromethyl)phenyl)- 1,2,3,4-tetrahydroquinolin- 5-yl)carbamate |
| 075 | 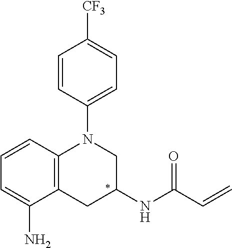 [SMILES: C=CC(=O)NC1Cc2c(N)cccc2N(c2ccc(C(F)(F)F)cc2)C1] | N-(5-amino-1-(4- (trifluoromethyl)phenyl)- 1,2,3,4-tetrahydroquinolin- 3-yl)acrylamide |
| 075- En1 | 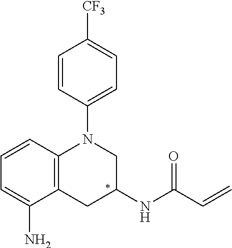 [SMILES: C=CC(=O)NC1Cc2c(N)cccc2N(c2ccc(C(F)(F)F)cc2)C1] | (R)- or (S)-N-(5-amino-1-(4- (trifluoromethyl)phenyl)- 1,2,3,4-tetrahydroquinolin- 3-yl)acrylamide |
| 075- En2 | 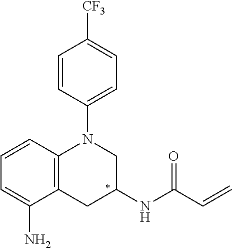 [SMILES: C=CC(=O)NC1Cc2c(N)cccc2N(c2ccc(C(F)(F)F)cc2)C1] | (R)- or (S)-N-(5-amino-1-(4- (trifluoromethyl)phenyl)- 1,2,3,4-tetrahydroquinolin- 3-yl)acrylamide |
| 076 | 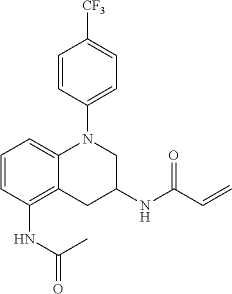 [SMILES: C=CC(=O)NC1Cc2c(NC(C)=O)cccc2N(c2ccc(C(F)(F)F)cc2)C1] | N-(5-acetamido-1-(4- (trifluoromethyl)phenyl)- 1,2,3,4-tetrahydroquinolin- 3-yl)acrylamide |
| 077 | 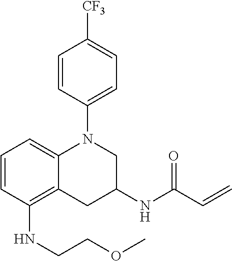 [SMILES: C=CC(=O)NC1Cc2c(NCCOC)cccc2N(c2ccc(C(F)(F)F)cc2)C1] | N-(5-((2-methoxyethyl)- amino)-1-(4- (trifluoromethyl)phenyl)- 1,2,3,4-tetrahydroquinolin- 3-yl)acrylamide |
| 078 | 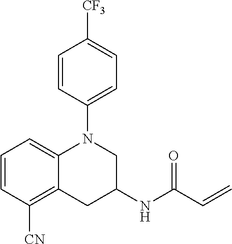 [SMILES: C=CC(=O)NC1Cc2c(C#N)cccc2N(c2ccc(C(F)(F)F)cc2)C1] | N-(5-cyano-1-(4- (trifluoromethyl)phenyl)- 1,2,3,4-tetrahydroquinolin- 3-yl)acrylamide |
| 079 | 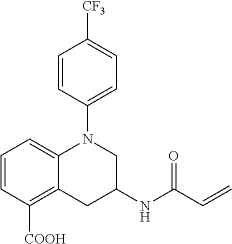 [SMILES: C=CC(=O)NC1Cc2c(C(=O)O)cccc2N(c2ccc(C(F)(F)F)cc2)C1] | 3-acrylamido-1-(4- (trifluoromethyl)phenyl)- 1,2,3,4-tetrahydro- quinoline-5-carboxylic acid |
| 080 | 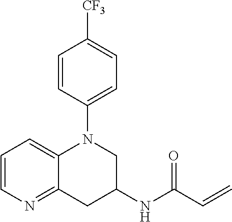 [SMILES: C=CC(=O)NC1Cc2ncccc2N(c2ccc(C(F)(F)F)cc2)C1] | N-(1-(4-(trifluoromethyl)- phenyl)-1,2,3,4-tetrahydro- 1,5-naphthyridin-3- yl)acrylamide |
| 081 | 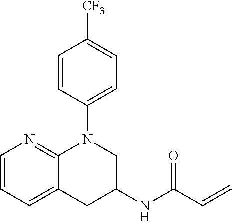 [SMILES: C=CC(=O)NC1Cc2cccnc2N(c2ccc(C(F)(F)F)cc2)C1] | N-(1-(4-(trifluoromethyl)- phenyl)-1,2,3,4-tetrahydro- 1,8-naphthyridin-3- yl)acrylamide |
| 082 | 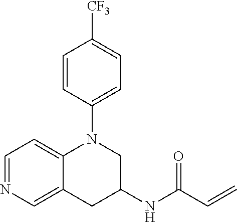 [SMILES: C=CC(=O)NC1Cc2cnccc2N(c2ccc(C(F)(F)F)cc2)C1] | N-(1-(4-(trifluoromethyl)- phenyl)-1,2,3,4-tetrahydro- 1,6-naphthyridin-3- yl)acrylamide |
| 083 | 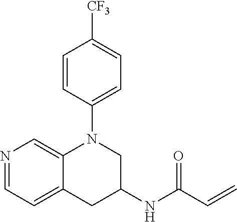 [SMILES: C=CC(=O)NC1Cc2ccncc2N(c2ccc(C(F)(F)F)cc2)C1] | N-(1-(4-(trifluoromethyl)- phenyl)-1,2,3,4-tetrahydro- 1,7-naphthyridin-3- yl)acrylamide |
| 084 | 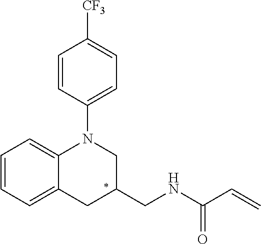 [SMILES: C=CC(=O)NCC1Cc2ccccc2N(c2ccc(C(F)(F)F)cc2)C1] | N-((1-(4-(trifluoromethyl)- phenyl)-1,2,3,4-tetrahydro- quinolin-3-yl)methyl)- acrylamide |
| 084- En1 | 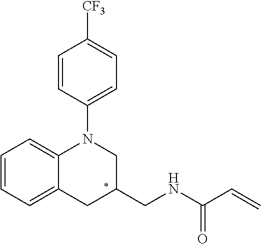 [SMILES: C=CC(=O)NCC1Cc2ccccc2N(c2ccc(C(F)(F)F)cc2)C1] | (R)- or (S)-N-((1-(4- (trifluoromethyl)-phenyl)- 1,2,3,4-tetrahydro-quinolin- 3-yl)methyl)-acrylamide |
| 084- En2 | 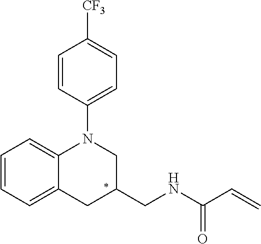 [SMILES: C=CC(=O)NCC1Cc2ccccc2N(c2ccc(C(F)(F)F)cc2)C1] | (R) or (S)-N-((1-(4- (trifluoromethyl)-phenyl)- 1,2,3,4-tetrahydro-quinolin- 3-yl)methyl)-acrylamide |
| 085 | 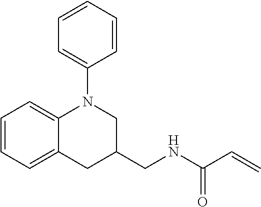 [SMILES: C=CC(=O)NCC1Cc2ccccc2N(c2ccccc2)C1] | N-((1-phenyl-1,2,3,4- tetrahydroquinolin-3- yl)methyl)acrylamide |
| 086 | 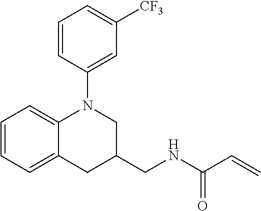 [SMILES: C=CC(=O)NCC1Cc2ccccc2N(c2cccc(C(F)(F)F)c2)C1] | N-((1-(3-(trifluoromethyl)- phenyl)-1,2,3,4-tetrahydro- quinolin-3-yl)methyl)- acrylamide |
| 087 | 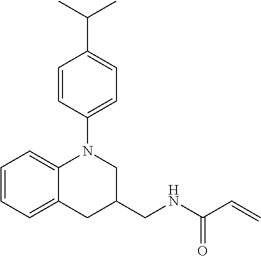 [SMILES: C=CC(=O)NCC1Cc2ccccc2N(c2ccc(C(C)C)cc2)C1] | N-((1-(4-isopropylphenyl)- 1,2,3,4-tetrahydroquinolin- 3-yl)methyl)acrylamide |
| 088 | 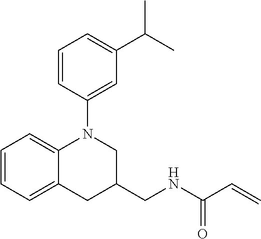 [SMILES: C=CC(=O)NCC1Cc2ccccc2N(c2cccc(C(C)C)c2)C1] | N-((1-(3-isopropylphenyl)- 1,2,3,4-tetrahydroquinolin- 3-yl)methyl)acrylamide |
| 089 | 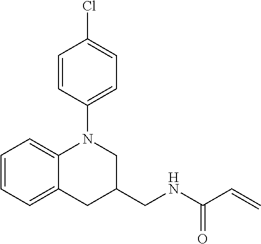 [SMILES: C=CC(=O)NCC1Cc2ccccc2N(c2ccc(Cl)cc2)C1] | N-((1-(4-chlorophenyl)- 1,2,3,4-tetrahydroquinolin- 3-yl)methyl)acrylamide |
| 090 | 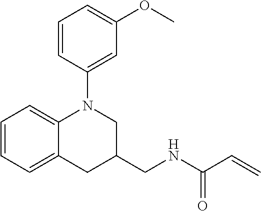 [SMILES: C=CC(=O)NCC1Cc2ccccc2N(c2cccc(OC)c2)C1] | N-((1-(3-methoxyphenyl)- 1,2,3,4-tetrahydroquinolin- 3-yl)methyl)acrylamide |
| 091 | 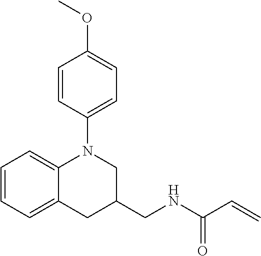 [SMILES: C=CC(=O)NCC1Cc2ccccc2N(c2ccc(OC)cc2)C1] | N-((1-(4-methoxyphenyl)- 1,2,3,4-tetrahydroquinolin- 3-yl)methyl)acrylamide |
| 092 | 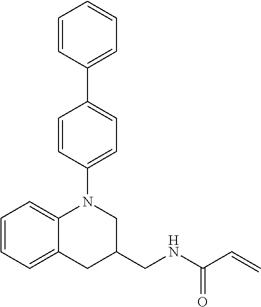 [SMILES: C=CC(=O)NCC1Cc2ccccc2N(c2ccc(-c3ccccc3)cc2)C1] | N-((1-([1,1′-biphenyl]-4-yl)- 1,2,3,4-tetrahydroquinolin- 3-yl)methyl)acrylamide |
| 093 | 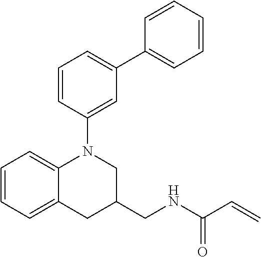 [SMILES: C=CC(=O)NCC1Cc2ccccc2N(c2cccc(-c3ccccc3)c2)C1] | N-((1-([1,1′-biphenyl]-3-yl)- 1,2,3,4-tetrahydroquinolin- 3-yl)methyl)acrylamide |
| 094 | 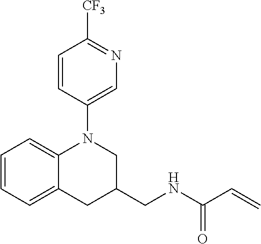 [SMILES: C=CC(=O)NCC1Cc2ccccc2N(c2ccc(C(F)(F)F)nc2)C1] | N-((1-(6-(trifluoromethyl)- pyridin-3-yl)-1,2,3,4- tetrahydroquinolin-3- yl)methyl)acrylamide |
| 095 | 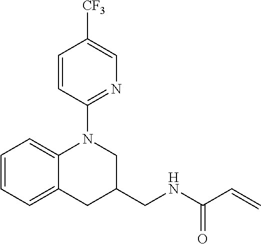 [SMILES: C=CC(=O)NCC1Cc2ccccc2N(c2ccc(C(F)(F)F)cn2)C1] | N-((1-(6-(trifluoromethyl)- pyridin-2n-yl)-1,2,3,4- tetrahydroquinolin-3- yl)methyl)acrylamide |
| 096 | 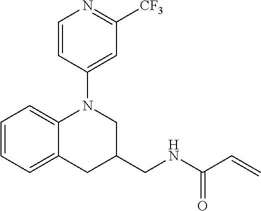 [SMILES: C=CC(=O)NCC1Cc2ccccc2N(c2ccnc(C(F)(F)F)c2)C1] | N-((1-(2-(trifluoromethyl)- pyridin-4-yl)-1,2,3,4- tetrahydroquinolin-3- yl)methyl)acrylamide |
| 097 | 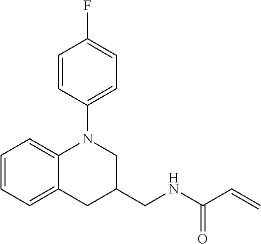 [SMILES: C=CC(=O)NCC1Cc2ccccc2N(c2ccc(F)cc2)C1] | N-((1-(4-fluorophenyl)- 1,2,3,4-tetrahydroquinolin- 3-yl)methyl)acrylamide |
| 098 | 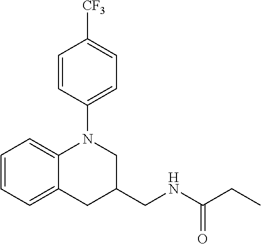 [SMILES: CCC(=O)NCC1Cc2ccccc2N(c2ccc(C(F)(F)F)cc2)C1] | N-((1-(4-(trifluoromethyl)- phenyl)-1,2,3,4- tetrahydroquinolin-3- yl)methyl)propionamide |
| 099 | 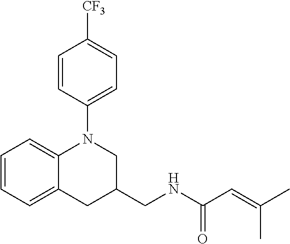 [SMILES: CC(C)=CC(=O)NCC1Cc2ccccc2N(c2ccc(C(F)(F)F)cc2)C1] | 3-methyl-N-((1-(4- (trifluoromethyl)phenyl)- 1,2,3,4-tetrahydroquinolin- 3-yl)methyl)but-2-enamide |
| 100 | 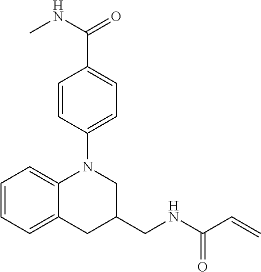 [SMILES: C=CC(=O)NCC1Cc2ccccc2N(c2ccc(C(=O)NC)cc2)C1] | 4-(3-(acrylamidomethyl)- 3,4-dihydroquinolin-1(2H)- yl)-N-methylbenzamide |
| 101 | 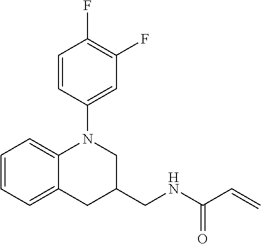 [SMILES: C=CC(=O)NCC1Cc2ccccc2N(c2ccc(F)c(F)c2)C1] | N-((1-(3,4-difluorophenyl)- 1,2,3,4-tetrahydroquinolin- 3-yl)methyl)acrylamide |
| 102 | 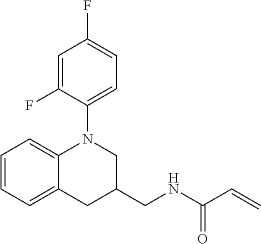 [SMILES: C=CC(=O)NCC1Cc2ccccc2N(c2ccc(F)cc2F)C1] | N-((1-(2,4-difluorophenyl)- 1,2,3,4-tetrahydroquinolin- 3-yl)methyl)acrylamide |
| 103 | 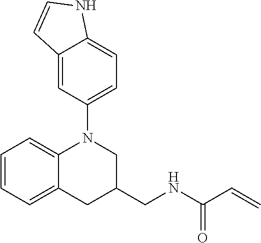 [SMILES: C=CC(=O)NCC1Cc2ccccc2N(c2ccc3[nH]ccc3c2)C1] | N-((1-(1H-indol-5-yl)- 1,2,3,4-tetrahydroquinolin- 3-yl)methyl)acrylamide |
| 104 | 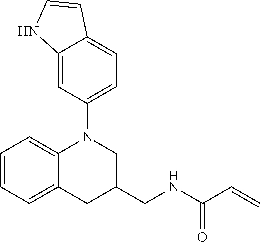 [SMILES: C=CC(=O)NCC1Cc2ccccc2N(c2ccc3cc[nH]c3c2)C1] | N-((1-(1H-indol-6-yl)- 1,2,3,4-tetrahydroquinolin- 3-yl)methyl)acrylamide |
| 105 | 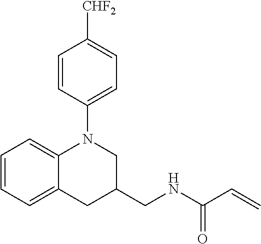 [SMILES: C=CC(=O)NCC1Cc2ccccc2N(c2ccc(C(F)F)cc2)C1] | N-((1-(4-(difluoromethyl)- phenyl)-1,2,3,4- tetrahydroquinolin-3- yl)methyl)acrylamide |
| 106 | 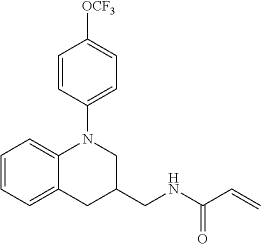 [SMILES: C=CC(=O)NCC1Cc2ccccc2N(c2ccc(OC(F)(F)F)cc2)C1] | N-((1-(4-(trifluoromethoxy)- phenyl)-1,2,3,4- tetrahydroquinolin-3- yl)methyl)acrylamide |
| 107 | 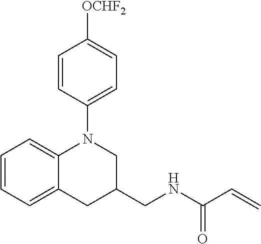 [SMILES: C=CC(=O)NCC1Cc2ccccc2N(c2ccc(OC(F)F)cc2)C1] | N-((1-(4- (difluoromethoxy)phenyl)- 1,2,3,4-tetrahydroquinolin- 3-yl)methyl)acrylamide |
| 108 | 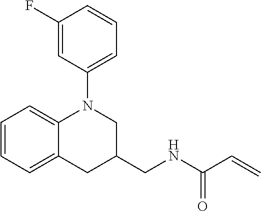 [SMILES: C=CC(=O)NCC1Cc2ccccc2N(c2cccc(F)c2)C1] | N-((1-(3-fluorophenyl)- 1,2,3,4-tetrahydroquinolin- 3-yl)methyl)acrylamide |
| 109 | 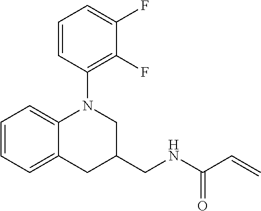 [SMILES: C=CC(=O)NCC1Cc2ccccc2N(c2cccc(F)c2F)C1] | N-((1-(2,3-difluorophenyl)- 1,2,3,4-tetrahydroquinolin- 3-yl)methyl)acrylamide |
| 110 | 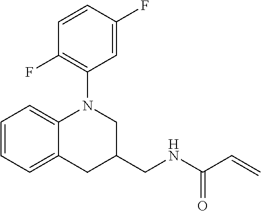 [SMILES: C=CC(=O)NCC1Cc2ccccc2N(c2cc(F)ccc2F)C1] | N-((1-(2,5-difluorophenyl)- 1,2,3,4-tetrahydroquinolin- 3-yl)methyl)acrylamide |
| 111 | 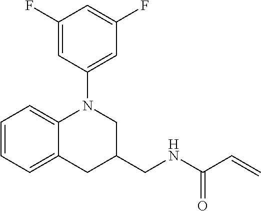 [SMILES: C=CC(=O)NCC1Cc2ccccc2N(c2cc(F)cc(F)c2)C1] | N-((1-(3,5-difluorophenyl)- 1,2,3,4-tetrahydroquinolin- 3-yl)methyl)acrylamide |
| 112 | 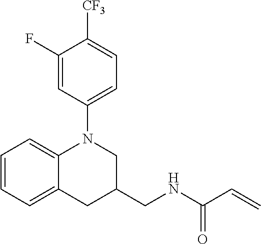 [SMILES: C=CC(=O)NCC1Cc2ccccc2N(c2ccc(C(F)(F)F)c(F)c2)C1] | N-((1-(3-fluoro-4- (trifluoromethyl)phenyl)- 1,2,3,4-tetrahydroquinolin- 3-yl)methyl)acrylamide |
| 113 | 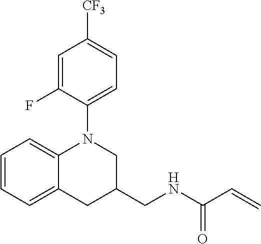 [SMILES: C=CC(=O)NCC1Cc2ccccc2N(c2ccc(C(F)(F)F)cc2F)C1] | N-((1-(2-fluoro-4- (trifluoromethyl)phenyl)- 1,2,3,4-tetrahydroquinolin- 3-yl)methyl)acrylamide |
| 114 | 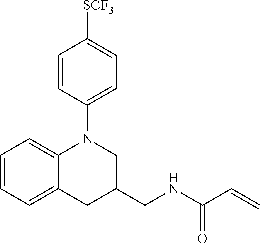 | N-((1-(4-((trifluoromethyl)- thio)phenyl)-1,2,3,4- tetrahydroquinolin-3- yl)methyl)acrylamide |
| 115 | 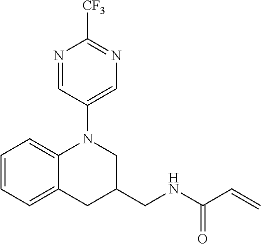 [SMILES: C=CC(=O)NCC1Cc2ccccc2N(c2cnc(C(F)(F)F)nc2)C1] | N-((1-(2-(trifluoromethyl)- pyrimidin-5-yl)-1,2,3,4- tetrahydroquinolin-3- yl)methyl)acrylamide |
| 116 | 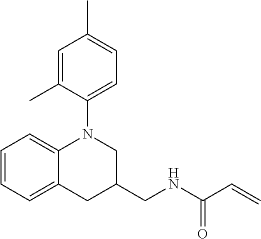 [SMILES: C=CC(=O)NCC1Cc2ccccc2N(c2ccc(C)cc2C)C1] | N-((1-(2,4-dimethylphenyl)- 1,2,3,4-tetrahydroquinolin- 3-yl)methyl)acrylamide |
| 117 | 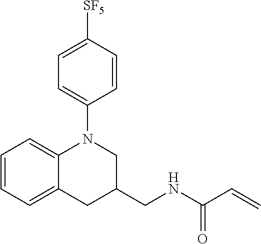 | N-((1-(4-(pentafluoro-λ⁶- sulfaneyl)phenyl)-1,2,3,4- tetrahydroquinolin-3- yl)methyl)acrylamide |
| 118 | 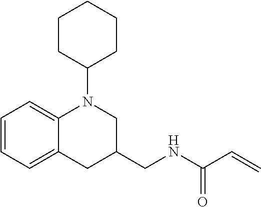 [SMILES: C=CC(=O)NCC1Cc2ccccc2N(C2CCCCC2)C1] | N-((1-cyclohexyl-1,2,3,4- tetrahydroquinolin-3- yl)methyl)acrylamide |
| 119 | 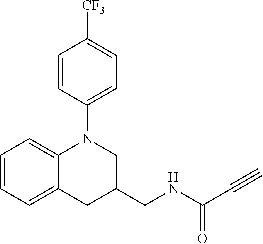 [SMILES: C#CC(=O)NCC1Cc2ccccc2N(c2ccc(C(F)(F)F)cc2)C1] | N-((1-(4-(trifluoromethyl)- phenyl)-1,2,3,4-tetrahydro- quinolin-3-yl)methyl)- propiolamide |
| 120 | 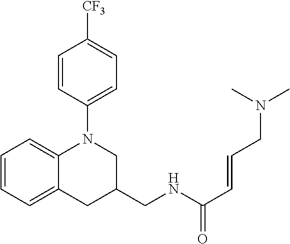 [SMILES: CN(C)C/C=C/C(=O)NCC1Cc2ccccc2N(c2ccc(C(F)(F)F)cc2)C1] | (E)-4-(dimethylamino)-N- ((1-(4-(trifluoromethyl)- phenyl)-1,2,3,4- tetrahydroquinolin-3- yl)methyl)but-2-enamide |
| 121 | 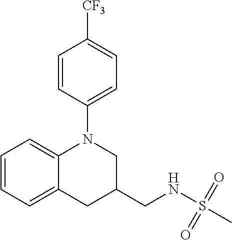 [SMILES: CS(=O)(=O)NCC1Cc2ccccc2N(c2ccc(C(F)(F)F)cc2)C1] | N-((1-(4-(trifluoromethyl)- phenyl)-1,2,3,4-tetrahydro- quinolin-3-yl)methyl)- methanesulfonamide |
| 122 | 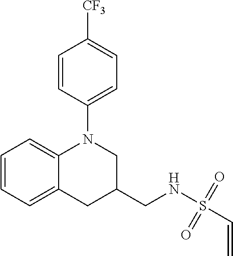 [SMILES: C=CS(=O)(=O)NCC1Cc2ccccc2N(c2ccc(C(F)(F)F)cc2)C1] | N-((1-(4-(trifluoromethyl)- phenyl)-1,2,3,4-tetrahydro- quinolin-3-yl)methyl)- ethenesulfonamide |
| 123 | 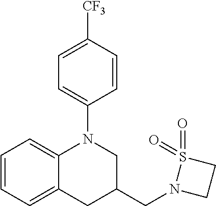 [SMILES: O=S1(=O)CCN1CC1Cc2ccccc2N(c2ccc(C(F)(F)F)cc2)C1] | 2-((1-(4-(trifluoromethyl)- phenyl)-1,2,3,4-tetrahydro- quinolin-3-yl)methyl)-1,2- thiazetidine-1,1-dioxide |
| 124 | 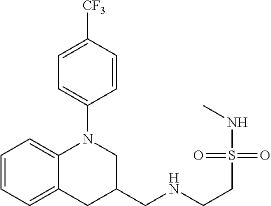 [SMILES: CNS(=O)(=O)CCNCC1Cc2ccccc2N(c2ccc(C(F)(F)F)cc2)C1] | N-methyl-2-(((1-(4- (trifluoromethyl)phenyl)- 1,2,3,4-tetrahydroquinolin- 3-yl)methyl)amino)ethane- 1-sulfonamide |
| 125 | 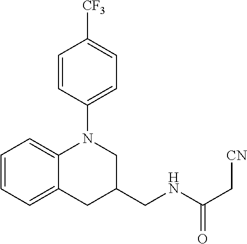 [SMILES: N#CCC(=O)NCC1Cc2ccccc2N(c2ccc(C(F)(F)F)cc2)C1] | 2-cyano-N-((1-(4- (trifluoromethyl)phenyl)- 1,2,3,4-tetrahydroquinolin- 3-yl)methyl)acetamide |
| 126 | 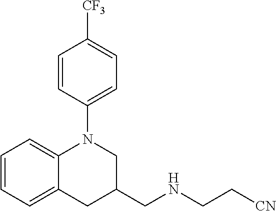 [SMILES: N#CCCNCC1Cc2ccccc2N(c2ccc(C(F)(F)F)cc2)C1] | 3-(((1-(4-(trifluoromethyl)- phenyl)-1,2,3,4-tetrahydro- quinolin-3-yl)methyl)- amino)propanenitrile |
| 127 | 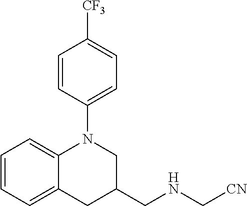 [SMILES: N#CCNCC1Cc2ccccc2N(c2ccc(C(F)(F)F)cc2)C1] | 2-(((1-(4-(trifluoromethyl)- phenyl)-1,2,3,4-tetrahydro- quinolin-3-yl)methyl)- amino)acetonitrile |
| 128 | 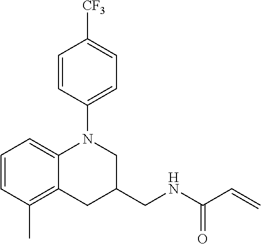 [SMILES: C=CC(=O)NCC1Cc2c(C)cccc2N(c2ccc(C(F)(F)F)cc2)C1] | N-((5-methyl-1-(4- (trifluoromethyl)phenyl)- 1,2,3,4-tetrahydroquinolin- 3-yl)methyl)acrylamide |
| 129 | 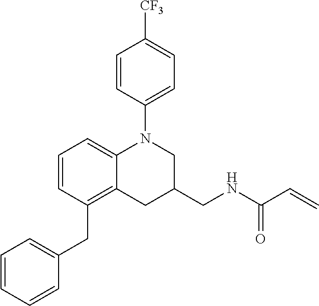 [SMILES: C=CC(=O)NCC1Cc2c(Cc3ccccc3)cccc2N(c2ccc(C(F)(F)F)cc2)C1] | N-((5-benzyl-1-(4- (trifluoromethyl)phenyl)- 1,2,3,4-tetrahydroquinolin- 3-yl)methyl)acrylamide |
| 130 | 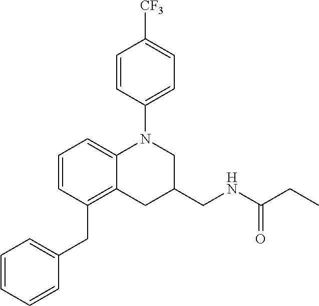 [SMILES: CCC(=O)NCC1Cc2c(Cc3ccccc3)cccc2N(c2ccc(C(F)(F)F)cc2)C1] | N-((5-benzyl-1-(4- (trifluoromethyl)phenyl)- 1,2,3,4-tetrahydroquinolin- 3-yl)methyl)propionamide |
| 131 | 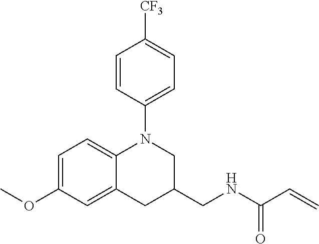 [SMILES: C=CC(=O)NCC1Cc2cc(OC)ccc2N(c2ccc(C(F)(F)F)cc2)C1] | N-((6-methoxy-1-(4- (trifluoromethyl)phenyl)- 1,2,3,4-tetrahydroquinolin- 3-yl)methyl)acrylamide |
| 132 | 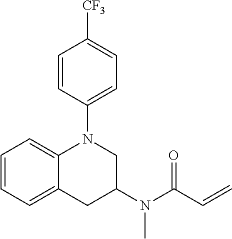 [SMILES: C=CC(=O)N(C)C1Cc2ccccc2N(c2ccc(C(F)(F)F)cc2)C1] | N-methyl-N-(1-(4- (trifluoromethyl)phenyl)- 1,2,3,4-tetrahydroquinolin- 3-yl)acrylamide |
| 133 | 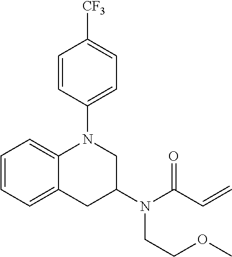 [SMILES: C=CC(=O)N(CCOC)C1Cc2ccccc2N(c2ccc(C(F)(F)F)cc2)C1] | N-(2-methoxyethyl)-N-(1- (4-(trifluoromethyl)phenyl)- 1,2,3,4-tetrahydroquinolin- 3-yl)acrylamide |
| 134 | 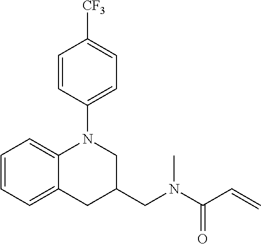 [SMILES: C=CC(=O)N(C)CC1Cc2ccccc2N(c2ccc(C(F)(F)F)cc2)C1] | N-methyl-N-((1-(4- (trifluoromethyl)phenyl)- 1,2,3,4-tetrahydroquinolin- 3-yl)methyl)acrylamide |
| 135 | 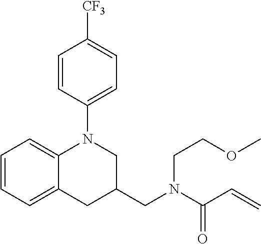 [SMILES: C=CC(=O)N(CCOC)CC1Cc2ccccc2N(c2ccc(C(F)(F)F)cc2)C1] | N-(2-methoxyethyl)-N-((1- (4-(trifluoromethyl)phenyl)- 1,2,3,4-tetrahydroquinolin- 3-yl)methyl)acrylamide |
| 136 | 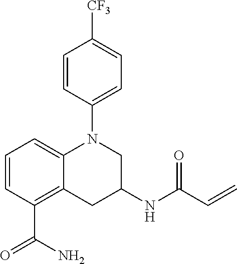 [SMILES: C=CC(=O)NC1Cc2c(C(N)=O)cccc2N(c2ccc(C(F)(F)F)cc2)C1] | 3-acrylamido-1-(4- (trifluoromethyl)phenyl)- 1,2,3,4-tetrahydro- quinoline-5-carboxamide |
| 137 | 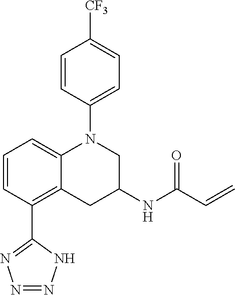 [SMILES: C=CC(=O)NC1Cc2c(-c3nnn[nH]3)cccc2N(c2ccc(C(F)(F)F)cc2)C1] | N-(5-(1H-tetrazol-5-yl)-1- (4-(trifluoromethyl)phenyl)- 1,2,3,4-tetrahydroquinolin- 3-yl)acrylamide |
| 138 | 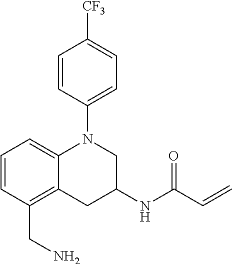 [SMILES: C=CC(=O)NC1Cc2c(CN)cccc2N(c2ccc(C(F)(F)F)cc2)C1] | N-(5-(aminomethyl)-1-(4- (trifluoromethyl)phenyl)- 1,2,3,4-tetrahydroquinolin- 3-yl)acrylamide |
| 139 | 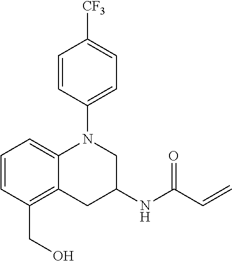 [SMILES: C=CC(=O)NC1Cc2c(CO)cccc2N(c2ccc(C(F)(F)F)cc2)C1] | N-(5-(hydroxymethyl)-1-(4- (trifluoromethyl)phenyl)- 1,2,3,4-tetrahydroquinolin- 3-yl)acrylamide |
| 140 | 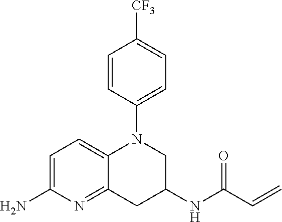 [SMILES: C=CC(=O)NC1Cc2nc(N)ccc2N(c2ccc(C(F)(F)F)cc2)C1] | N-(6-amino-1-(4- (trifluoromethyl)phenyl)- 1,2,3,4-tetrahydro-1,5- naphthyridin-3-yl)acryl- amide |
| 141 | 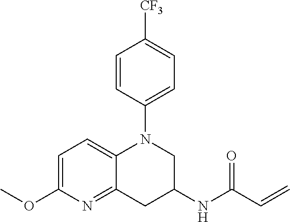 [SMILES: C=CC(=O)NC1Cc2nc(OC)ccc2N(c2ccc(C(F)(F)F)cc2)C1] | N-(6-methoxy-1-(4- (trifluoromethyl)phenyl)- 1,2,3,4-tetrahydro-1,5- naphthyridin-3-yl)acryl- amide |
| 142 | 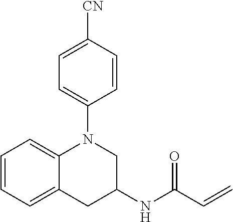 [SMILES: C=CC(=O)NC1Cc2ccccc2N(c2ccc(C#N)cc2)C1] | N-(1-(4-cyanophenyl)- 1,2,3,4-tetrahydroquinolin- 3-yl)acrylamide |
| 143 | 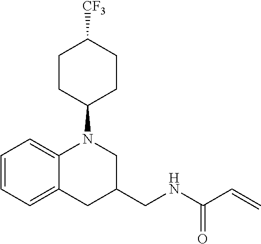 [SMILES: C=CC(=O)NCC1Cc2ccccc2N([C@H]2CC[C@H](C(F)(F)F)CC2)C1] | trans-N-((1-(4- (trifluoromethyl)cyclohexyl)- 1,2,3,4-tetrahydroquinolin- 3-yl)methyl)acrylamide |
| 144 | 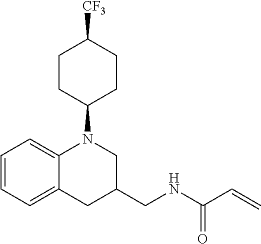 [SMILES: C=CC(=O)NCC1Cc2ccccc2N([C@H]2CC[C@@H](C(F)(F)F)CC2)C1] | cis-N-((1-(4- (trifluoromethyl)cyclohexyl)- 1,2,3,4-tetrahydroquinolin- 3-yl)methyl)acrylamide |
| 145 | 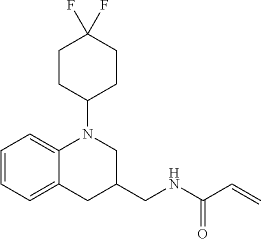 [SMILES: C=CC(=O)NCC1Cc2ccccc2N(C2CCC(F)(F)CC2)C1] | N-((1-(4,4- difluorocyclohexyl)-1,2,3,4- tetrahydroquinolin-3- yl)methyl)acrylamide |
| 146 | 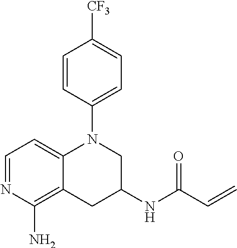 [SMILES: C=CC(=O)NC1Cc2c(ccnc2N)N(c2ccc(C(F)(F)F)cc2)C1] | N-(5-amino-1-(4- (trifluoromethyl)phenyl)- 1,2,3,4-tetrahydro-1,6- naphthyridin-3-yl)acryl- amide |
| 147 | 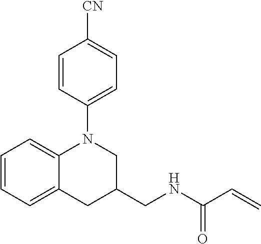 [SMILES: C=CC(=O)NCC1Cc2ccccc2N(c2ccc(C#N)cc2)C1] | N-((1-(4-cyanophenyl)- 1,2,3,4-tetrahydroquinolin- 3-yl)methyl)acrylamide |
| 148 | 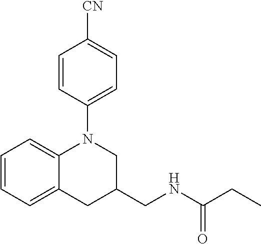 [SMILES: CCC(=O)NCC1Cc2ccccc2N(c2ccc(C#N)cc2)C1] | N-((1-(4-cyanophenyl)- 1,2,3,4-tetrahydroquinolin- 3-yl)methyl)propionamide |
| 149 | 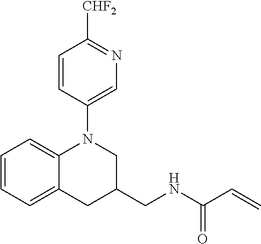 [SMILES: C=CC(=O)NCC1Cc2ccccc2N(c2ccc(C(F)F)nc2)C1] | N-((1-(6- (difluoromethyl)pyridin-3- yl)-1,2,3,4- tetrahydroquinolin-3- yl)methyl)acrylamide |
| 150 | 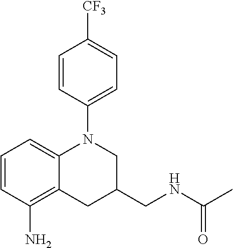 [SMILES: CC(=O)NCC1Cc2c(N)cccc2N(c2ccc(C(F)(F)F)cc2)C1] | N-((5-amino-1-(4- (trifluoromethyl)phenyl)- 1,2,3,4-tetrahydroquinolin- 3-yl)methyl)acetamide |
| 151 | 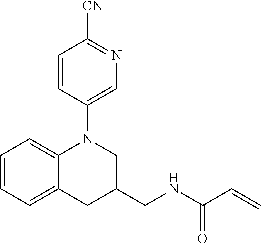 [SMILES: C=CC(=O)NCC1Cc2ccccc2N(c2ccc(C#N)nc2)C1] | N-((1-(6-cyanopyridin-3-yl)- 1,2,3,4-tetrahydroquinolin- 3-yl)methyl)acrylamide |
| 152 | 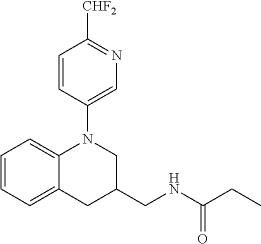 [SMILES: CCC(=O)NCC1Cc2ccccc2N(c2ccc(C(F)F)nc2)C1] | N-((1-(6- (difluoromethyl)pyridin-3- yl)-1,2,3,4- tetrahydroquinolin-3- yl)methyl)propionamide |
| 153 | 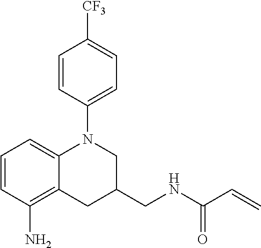 [SMILES: C=CC(=O)NCC1Cc2c(N)cccc2N(c2ccc(C(F)(F)F)cc2)C1] | N-((5-amino-1-(4- (trifluoromethyl)phenyl)- 1,2,3,4-tetrahydroquinolin- 3-yl)methyl)acrylamide |
| 154 | 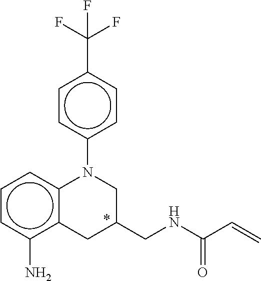 | (R)- or (S)-N-((5-amino-1- (4-(trifluoromethyl)phenyl)- 1,2,3,4-tetrahydroquinolin- 3-yl)methyl)acrylamide |
| 153- En2 | 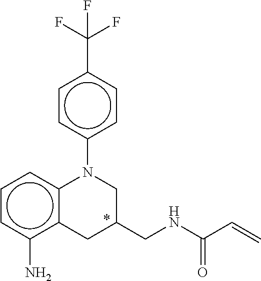 | (R)- or (S)-N-((5-amino-1- (4-(trifluoromethyl)phenyl)- 1,2,3,4-tetrahydroquinolin- 3-yl)methyl)acrylamide |
| 154 | 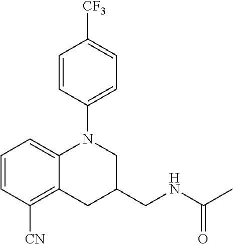 [SMILES: CC(=O)NCC1Cc2c(C#N)cccc2N(c2ccc(C(F)(F)F)cc2)C1] | N-((5-cyano-1-(4- (trifluoromethyl)phenyl)- 1,2,3,4-tetrahydroquinolin- 3-yl)methyl)acetamide |
| 155 | 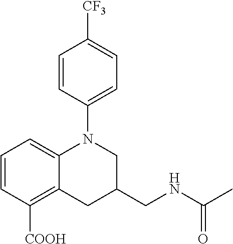 [SMILES: CC(=O)NCC1Cc2c(C(=O)O)cccc2N(c2ccc(C(F)(F)F)cc2)C1] | 3-(acetamidomethyl)-1-(4- (trifluoromethyl)phenyl)- 1,2,3,4-tetrahydro- quinoline-5-carboxylic acid |
| 156 | 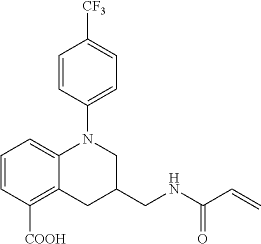 [SMILES: C=CC(=O)NCC1Cc2c(C(=O)O)cccc2N(c2ccc(C(F)(F)F)cc2)C1] | 3-(acrylamidomethyl)-1-(4- (trifluoromethyl)phenyl)- 1,2,3,4-tetrahydro- quinoline-5-carboxylic acid |
| 156- En1 | 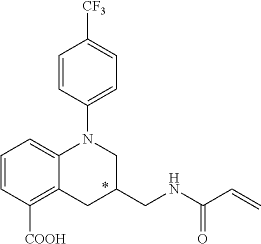 [SMILES: C=CC(=O)NCC1Cc2c(C(=O)O)cccc2N(c2ccc(C(F)(F)F)cc2)C1] | (R)- or (S)-3- (acrylamidomethyl)-1-(4- (trifluoromethyl)phenyl)- 1,2,3,4-tetrahydro- quinoline-5-carboxylic acid |
| 156- En2 | 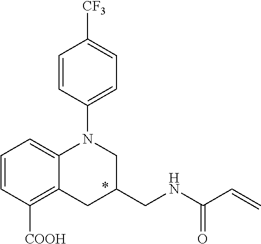 [SMILES: C=CC(=O)NCC1Cc2c(C(=O)O)cccc2N(c2ccc(C(F)(F)F)cc2)C1] | (R)- or (S)-3- (acrylamidomethyl)-1-(4- (trifluoromethyl)phenyl)- 1,2,3,4-tetrahydro- quinoline-5-carboxylic acid |
| 157 | 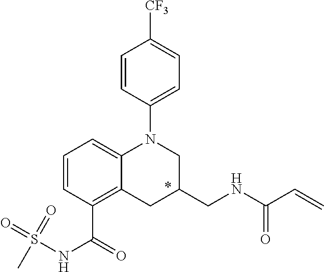 [SMILES: C=CC(=O)NCC1Cc2c(C(=O)NS(C)(=O)=O)cccc2N(c2ccc(C(F)(F)F)cc2)C1] | 3-(acrylamidomethyl)-N- (methylsulfonyl)-1-(4- (trifluoromethyl)phenyl)- 1,2,3,4-tetrahydro- quinoline-5-carboxamide |
| 157- En1 | 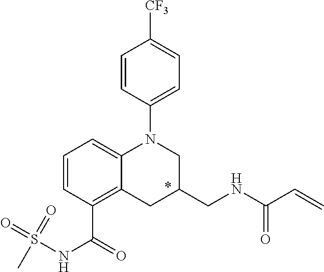 [SMILES: C=CC(=O)NCC1Cc2c(C(=O)NS(C)(=O)=O)cccc2N(c2ccc(C(F)(F)F)cc2)C1] | (R)- or (S)-3- (acrylamidomethyl)-N- (methylsulfonyl)-1-(4- (trifluoromethyl)phenyl)- 1,2,3,4-tetrahydro- quinoline-5-carboxamide |
| 157- En2 | 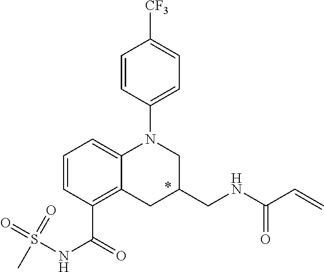 [SMILES: C=CC(=O)NCC1Cc2c(C(=O)NS(C)(=O)=O)cccc2N(c2ccc(C(F)(F)F)cc2)C1] | (R)- or (S)-3- (acrylamidomethyl)-N- (methylsulfonyl)-1-(4- (trifluoromethyl)phenyl)- 1,2,3,4-tetrahydro- quinoline-5-carboxamide |
| 158 | 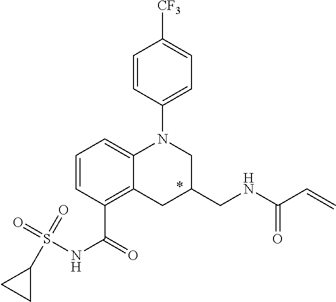 [SMILES: C=CC(=O)NCC1Cc2c(C(=O)NS(=O)(=O)C3CC3)cccc2N(c2ccc(C(F)(F)F)cc2)C1] | 3-(acrylamidomethyl)-N- (cyclopropylsulfonyl)-1-(4- (trifluoromethyl)phenyl)- 1,2,3,4-tetrahydro- quinoline-5-carboxamide |
| 158- En1 | 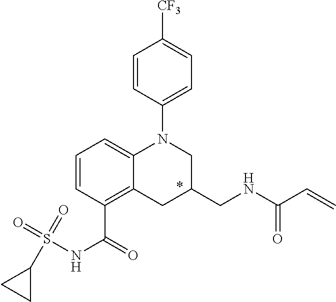 [SMILES: C=CC(=O)NCC1Cc2c(C(=O)NS(=O)(=O)C3CC3)cccc2N(c2ccc(C(F)(F)F)cc2)C1] | (R)- or (S)-3- (acrylamidomethyl)-N- (cyclopropylsulfonyl)-1-(4- (trifluoromethyl)phenyl)- 1,2,3,4-tetrahydro- quinoline-5-carboxamide |
| 158- En2 | 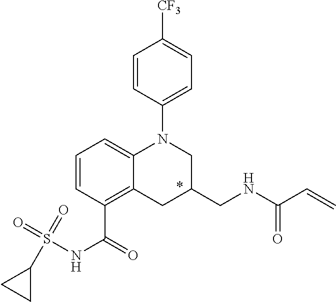 [SMILES: C=CC(=O)NCC1Cc2c(C(=O)NS(=O)(=O)C3CC3)cccc2N(c2ccc(C(F)(F)F)cc2)C1] | (R)- or (S)-3- (acrylamidomethyl)-N- (cyclopropylsulfonyl)-1-(4- (trifluoromethyl)phenyl)- 1,2,3,4-tetrahydro- quinoline-5-carboxamide |
| 159 | 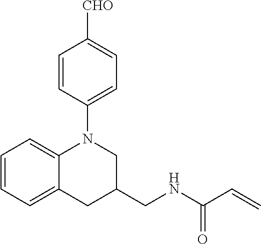 [SMILES: C=CC(=O)NCC1Cc2ccccc2N(c2ccc(C=O)cc2)C1] | N-((1-(4-formylphenyl)- 1,2,3,4-tetrahydroquinolin- 3-yl)methyl)acrylamide |
| 160 | 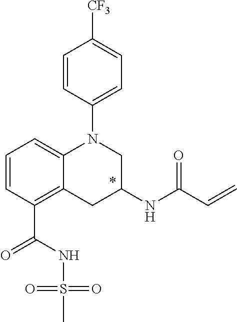 [SMILES: C=CC(=O)NC1Cc2c(C(=O)NS(C)(=O)=O)cccc2N(c2ccc(C(F)(F)F)cc2)C1] | 3-acrylamido-N-(methyl- sulfonyl)-1-(4-(trifluoro- methyl)phenyl)-1,2,3,4- tetrahydroquinoline-5- carboxamide |
| 160- En1 | 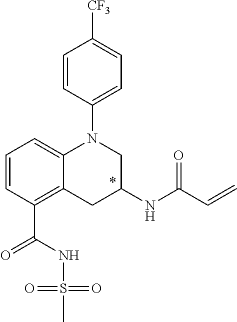 [SMILES: C=CC(=O)NC1Cc2c(C(=O)NS(C)(=O)=O)cccc2N(c2ccc(C(F)(F)F)cc2)C1] | (R)- or (S)-3-acrylamido-N- (methyl-sulfonyl)-1-(4- (trifluoro-methyl)phenyl)- 1,2,3,4-tetrahydroquinoline- 5-carboxamide |
| 160- En2 | 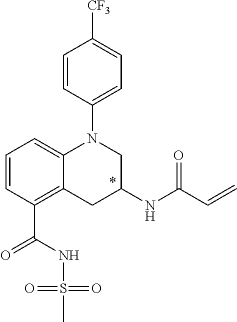 [SMILES: C=CC(=O)NC1Cc2c(C(=O)NS(C)(=O)=O)cccc2N(c2ccc(C(F)(F)F)cc2)C1] | (R)- or (S)-3-acrylamido-N- (methyl-sulfonyl)-1-(4- (trifluoro-methyl)phenyl)- 1,2,3,4-tetrahydroquinoline- 5-carboxamide |
| 161 | 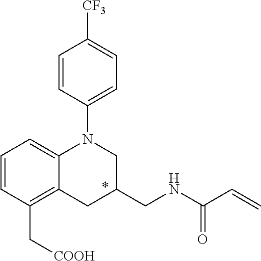 [SMILES: C=CC(=O)NCC1Cc2c(CC(=O)O)cccc2N(c2ccc(C(F)(F)F)cc2)C1] | (2-(3-(acrylamidomethyl)-1- (4-(trifluoromethyl)phenyl)- 1,2,3,4-tetrahydroquinolin- 5-yl)acetic acid |
| 161- En1 | 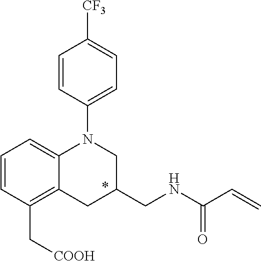 [SMILES: C=CC(=O)NCC1Cc2c(CC(=O)O)cccc2N(c2ccc(C(F)(F)F)cc2)C1] | (R)- or (S)-2-(3- (acrylamidomethyl)-1-(4- (trifluoromethyl)phenyl)- 1,2,3,4-tetrahydroquinolin- 5-yl)acetic acid |
| 161- En2 | 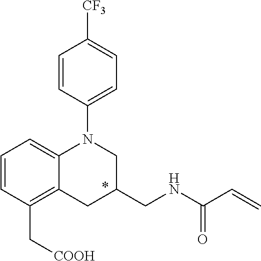 [SMILES: C=CC(=O)NCC1Cc2c(CC(=O)O)cccc2N(c2ccc(C(F)(F)F)cc2)C1] | (R)- or (S)-2-(3- (acrylamidomethyl)-1-(4- (trifluoromethyl)phenyl)- 1,2,3,4-tetrahydroquinolin- 5-yl)acetic acid |
| 162 | 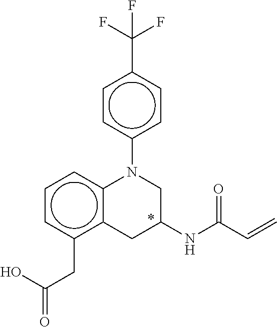 | 2-(3-acrylamido-1-(4- (trifluoromethyl)phenyl)- 1,2,3,4-tetrahydroquinolin- 5-yl)acetic acid |
| 162- En1 | 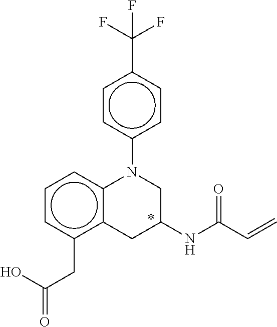 | (R)- or (S)-2-(3-acrylamido- 1-(4- (trifluoromethyl)phenyl)- 1,2,3,4-tetrahydroquinolin- 5-yl)acetic acid |
| 162- En2 | 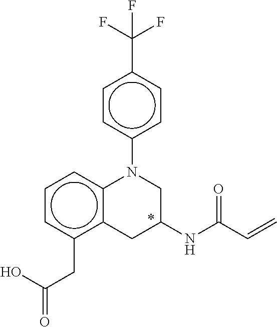 | (R)- or (S)-2-(3-acrylamido- 1-(4- (trifluoromethyl)phenyl)- 1,2,3,4-tetrahydroquinolin- 5-yl)acetic acid |
| 163 | 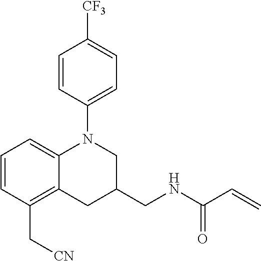 [SMILES: C=CC(=O)NCC1Cc2c(CC#N)cccc2N(c2ccc(C(F)(F)F)cc2)C1] | N-((5-(cyanomethyl)-1-(4- (trifluoromethyl)phenyl)- 1,2,3,4-tetrahydroquinolin- 3-yl)methyl)acrylamide |
| 164 | 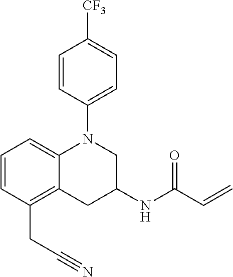 [SMILES: C=CC(=O)NC1Cc2c(CC#N)cccc2N(c2ccc(C(F)(F)F)cc2)C1] | N-(5-cyanomethyl)-1-(4- (trifluoromethyl)phenyl)- 1,2,3,4-tetrahydroquinolin- 3-yl)acrylamide |
| 165 | 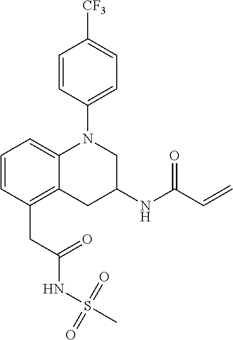 [SMILES: C=CC(=O)NC1Cc2c(CC(=O)NS(C)(=O)=O)cccc2N(c2ccc(C(F)(F)F)cc2)C1] | N-(5-(2- (methylsulfonamido)-2- oxoethyl)-1-(4-(trifluoro- methyl)phenyl)-1,2,3,4- tetrahydroquinolin-3- yl)acrylamide |
| 166 | 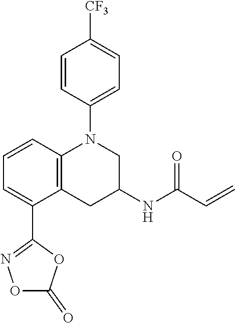 [SMILES: C=CC(=O)NC1Cc2c(-c3noc(=O)o3)cccc2N(c2ccc(C(F)(F)F)cc2)C1] | N-(5-(5-oxo-4,5-dihydro- 1,2,4-oxadiazol-3-yl)-1-(4- (trifluoromethyl)phenyl)- 1,2,3,4-tetrahydroquinolin- 3-yl)acrylamide |
| 166- En1 | 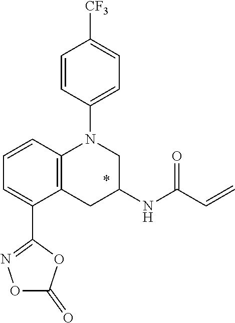 [SMILES: C=CC(=O)NC1Cc2c(-c3noc(=O)o3)cccc2N(c2ccc(C(F)(F)F)cc2)C1] | (R)- or (S)-N-(5-(5-oxo-4,5- dihydro-1,2,4-oxadiazol-3- yl)-1-(4- (trifluoromethyl)phenyl)- 1,2,3,4-tetrahydroquinolin- 3-yl)acrylamide |
| 166- En2 | 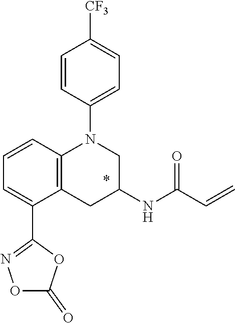 [SMILES: C=CC(=O)NC1Cc2c(-c3noc(=O)o3)cccc2N(c2ccc(C(F)(F)F)cc2)C1] | (R)- or (S)-N-(5-(5-oxo-4,5- dihydro-1,2,4-oxadiazol-3- yl)-1-(4- (trifluoromethyl)phenyl)- 1,2,3,4-tetrahydroquinolin- 3-yl)acrylamide |
| 167 | 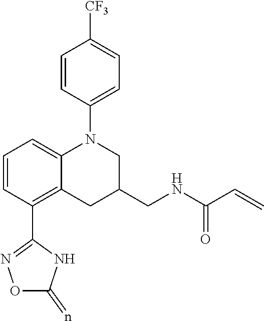 | N-((5-(5-oxo-4,5-dihydro- 1,2,4-oxadiazol-3-yl)-1-(4- (trifluoromethyl)phenyl)- 1,2,3,4-tetrahydroquinolin- 3-yl)methyl)acrylamide |
| 168 | 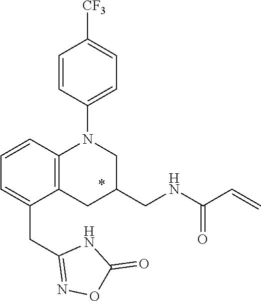 [SMILES: C=CC(=O)NCC1Cc2c(Cc3noc(=O)[nH]3)cccc2N(c2ccc(C(F)(F)F)cc2)C1] | N-((5-((5-oxo-4,5-dihydro- 1,2,4-oxadiazol-3-yl)- methyl)-1-(4-trifluoro- methyl)phenyl)-1,2,3,4- tetrahydroquinolin-3- yl)methyl)acrylamide |
| 168- En1 | 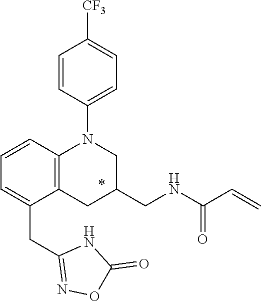 [SMILES: C=CC(=O)NCC1Cc2c(Cc3noc(=O)[nH]3)cccc2N(c2ccc(C(F)(F)F)cc2)C1] | (R)- or (S)-N-((5-((5-oxo- 4,5-dihydro-1,2,4-oxadiazol- 3-yl)-methyl)-1-(4- (trifluoro-methyl)phenyl)- 1,2,3,4-tetrahydroquinolin- 3-yl)methyl)acrylamide |
| 168- En2 | 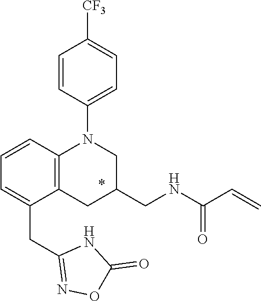 [SMILES: C=CC(=O)NCC1Cc2c(Cc3noc(=O)[nH]3)cccc2N(c2ccc(C(F)(F)F)cc2)C1] | (R)- or (S)-N-((5-((5-oxo- 4,5-dihydro-1,2,4-oxadiazol- 3-yl)-methyl)-1-(4- (trifluoro-methyl)phenyl)- 1,2,3,4-tetrahydroquinolin- 3-yl)methyl)acrylamide |
| 169 | 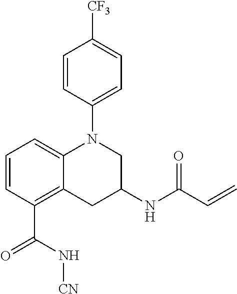 [SMILES: C=CC(=O)NC1Cc2c(C(=O)NC#N)cccc2N(c2ccc(C(F)(F)F)cc2)C1] | 3-acrylamido-N-cyano-1-(4- (trifluoromethyl)phenyl)- 1,2,3,4-tetrahydro- quinoline-5-carboxamide |
| 170 (Int-10) | 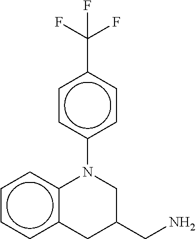 | (1-(4- (trifluoromethyl)phenyl)- 1,2,3,4-tetrahydroquinolin- 3-yl)methanamine |
| 171 (Int-3) | 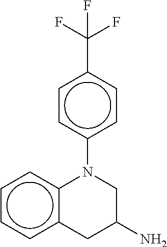 | 1-(4- (trifluoromethyl)phenyl)- 1,2,3,4-tetrahydroquinolin- 3-amine |
| 172 | 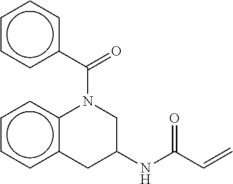 | N-(1-benzoyl-1,2,3,4- tetrahydroquinolin-3- yl)acrylamide |
| 173 | 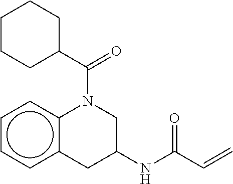 | N-(1- (cyclohexanecarbonyl)- 1,2,3,4-tetrahydroquinolin- 3-yl)acrylamide |
| 174 | 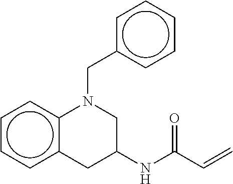 | N-(1-benzyl-1,2,3,4- tetrahydroquinolin-3- yl)acrylamide |
| 175 | 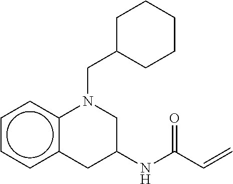 | N-(1-(cyclohexylmethyl)- 1,2,3,4-tetrahydroquinolin- 3-yl)acrylamide |
| 176 | 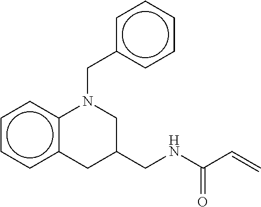 | N-((1-benzyl-1,2,3,4- tetrahydroquinolin-3- yl)methyl)acrylamide |
| 177 | 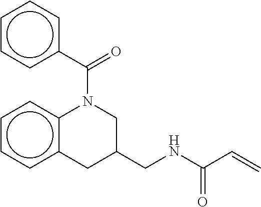 | N-((1-benzoyl-1,2,3,4- tetrahydroquinolin-3- yl)methyl)acrylamide |
| 178 | 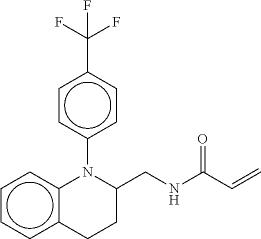 | N-((1-(4- (trifluoromethyl)phenyl)- 1,2,3,4-tetrahydroquinolin- 2-yl)methyl)acrylamide |
| 179 | 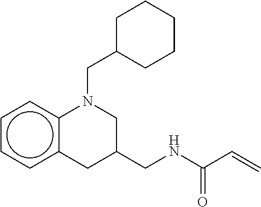 | N-((1-(cyclohexylmethyl)- 1,2,3,4-tetrahydroquinolin- 3-yl)methyl)acrylamide |
| 180 | 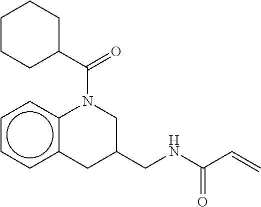 | N-((1- (cyclohexanecarbonyl)- 1,2,3,4-tetrahydroquinolin- 3-yl)methyl)acrylamide |
| 181 | 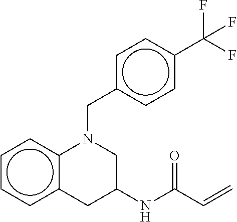 | N-(1-(4- (trifluoromethyl)benzyl)- 1,2,3,4-tetrahydroquinolin- 3-yl)acrylamide |
| 182 | 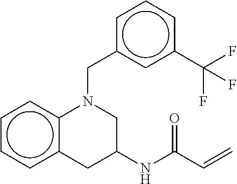 | N-(1-(3- (trifluoromethyl)benzyl)- 1,2,3,4-tetrahydroquinolin- 3-yl)acrylamide |
| 183 | 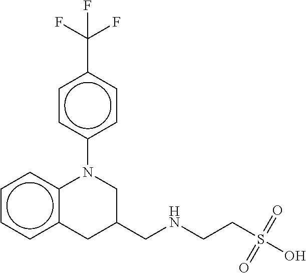 | 2-(((1-(4- (trifluoromethyl)phenyl)- 1,2,3,4-tetrahydroquinolin- 3-yl)methyl)amino)ethane- 1-sulfonic acid |
| 184 | 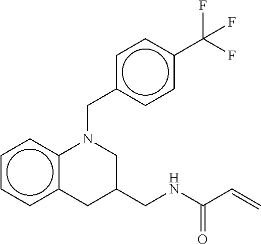 | N-((1-(4- (trifluoromethyl)benzyl)- 1,2,3,4-tetrahydroquinolin- 3-yl)methyl)acrylamide |
| 185 | 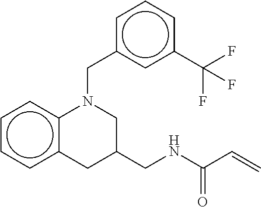 | N-((1-(3- (trifluoromethyl)benzyl)- 1,2,3,4-tetrahydroquinolin- 3-yl)methyl)acrylamide |
| 186 | 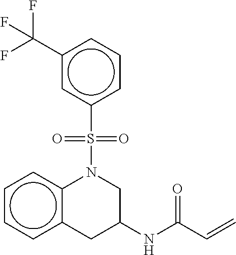 | N-(1-((3- (trifluoromethyl)phenyl) sulfonyl)-1,2,3,4- tetrahydroquinolin-3- yl)acrylamide |
| 187 | 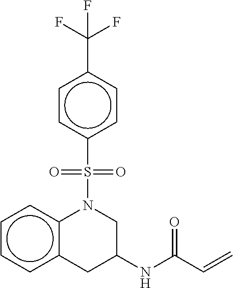 | N-(1-((4- (trifluoromethyl)phenyl) sulfonyl)-1,2,3,4- tetrahydroquinolin-3- yl)acrylamide |
| 188 | 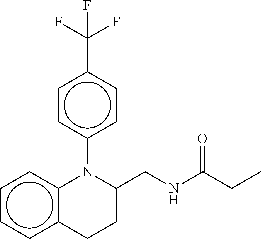 | N-((1-(4- (trifluoromethyl)phenyl)- 1,2,3,4-tetrahydroquinolin- 2-yl)methyl)propionamide |
| 189 | 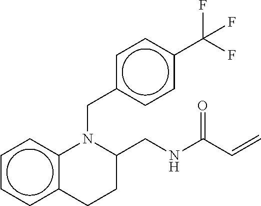 | N-((1-(4- (trifluoromethyl)benzyl)- 1,2,3,4-tetrahydroquinolin- 2-yl)methyl)acrylamide |
| 190 | 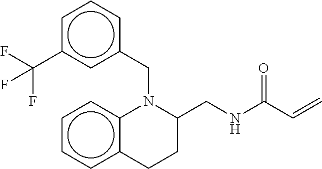 | N-((1-(3- (trifluoromethyl)benzyl)- 1,2,3,4-tetrahydroquinolin- 2-yl)methyl)acrylamide |
| 191 | 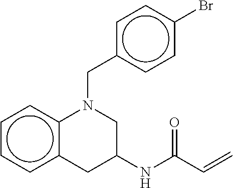 | N-(1-(4-bromobenzyl)- 1,2,3,4-tetrahydroquinolin- 3-yl)acrylamide |
| 192 | 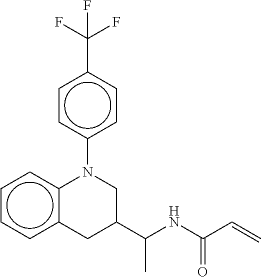 | N-(1-(1-(4- (trifluoromethyl)phenyl)- 1,2,3,4-tetrahydroquinolin- 3-yl)ethyl)acrylamide |
| 193 | 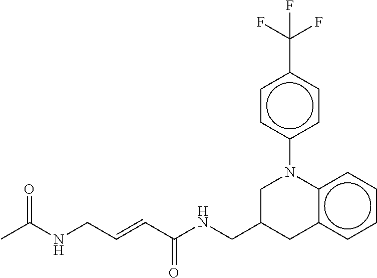 | (E)-4-acetamido-N-((1-(4- (trifluoromethyl)phenyl)- 1,2,3,4-tetrahydroquinolin- 3-yl)methyl)but-2-enamide |
| 194 | 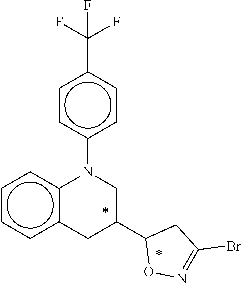 | 3-bromo-5-(1-(4- (trifluoromethyl)phenyl)- 1,2,3,4-tetrahydroquinolin- 3-yl)-4,5-dihydroisoxazole |
| 194(Dia1)- En1 | 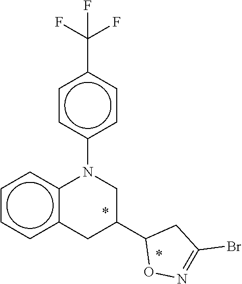 | (R)- or (S)-3-bromo-5-(1-(4- (trifluoromethyl)phenyl)- 1,2,3,4-tetrahydroquinolin- 3-yl)-4,5-dihydroisoxazole |
| 194(Dia1)- En2 | 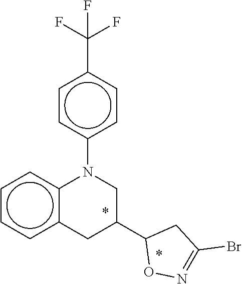 | (R)- or (S)-3-bromo-5-(1-(4- (trifluoromethyl)phenyl)- 1,2,3,4-tetrahydroquinolin- 3-yl)-4,5-dihydroisoxazole |
| 194(Dia2)- En1 | 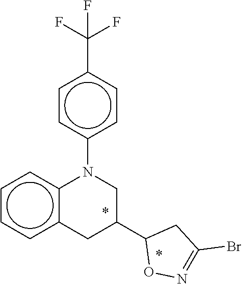 | (R)- or (S)-3-bromo-5-(1-(4- (trifluoromethyl)phenyl)- 1,2,3,4-tetrahydroquinolin- 3-yl)-4,5-dihydroisoxazole |
| 194(Dia2)- En2 | 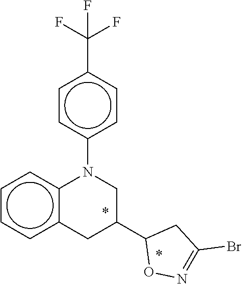 | (R)- or (S)-3-bromo-5-(1-(4- (trifluoromethyl)phenyl)- 1,2,3,4-tetrahydroquinolin- 3-yl)-4,5-dihydroisoxazole |

The following examples are provided for the purpose of illustrating the present disclosure and by no means should be interpreted to limit the scope of the present disclosure.

Examples: Part A represent the preparation of the compounds whereas Part B represents the pharmacological examples.

Part A: Experimental Chemistry Procedures

All starting materials which are not explicitly described were either commercially available (the details of suppliers such as for example Acros, Avocado, Aldrich, Fluka, FluoroChem, MatrixScientific, Maybridge, Merck, Sigma, etc. can be found in the SciFinder® Database for example) or the synthesis thereof has already been described precisely in the specialist literature (experimental guidelines can be found in the Reaxys® Database or the SciFinder® Database respectively, for example) or can be prepared using the conventional methods known to the person skilled in the art.

The reactions were, if necessary, carried out under an inert atmosphere (mostly argon and N₂). The number of equivalents of reagents and the amounts of solvents employed as well as the reaction temperatures and times can vary slightly between different reactions carried out by analogous methods. The work-up and purification methods were adapted according to the characteristic properties of each compound and can vary slightly for analogous methods. The yields of the compounds prepared are not optimized.

The indication, equivalents” (“eq.” or “eq” or “equiv.”) means molar equivalents, RT″ or “rt” means room temperature T (23±7° C.), M″ are indications of concentration in mol/l, sol.” means solution, “conc.” means concentrated. The mixing ratios of solvents are usually stated in the volume/volume ratio.

To perform reactions under microwave radiation a CEM Microwave (Discover SP) was employed (Heating rate: 2-6° C./sec; Temperature: 30-300° C. volume-independent infrared (IR) and 80-300° C. Fiber optic (FO) temperature measurement; Pressure: 0-435 psi, ActiVent™ technology; Power: 0-300 W; Magnetron frequency: 2450 MHz; Reaction agitation: electromagnetic stirring; Air Cooling: 25 psi (20 L/min flow); System control: Synergy™ software).

Key analytical characterization was carried out by means of ¹H-NMR spectroscopy and/or mass spectrometry (MS, m/z for [M+H]⁺ and/or [M−H]⁻) for all the exemplary compounds and selected intermediate products. In certain cases, where e.g. regioisomers and/or diatereomers could be/were formed during the reaction, additional analytics, such as, e.g. ¹³C NMR and NOE (nuclear overhauser effect) NMR experiments were in some cases performed.

Analytical instruments employed were e.g. for NMR analysis a BRUKER AVANCE 400 MHz (Software Topspin) or a VARIAN MR 400 MHz (VNMRJ Sofware) machine was employed. For LC/MS analysis e.g. an Acquity UPLC H-Class, Mass: Acquity SQD2 Detector (ESI), an Acquity UPLC, Mass: Quatro premier XE Detector (ESI), an Acquity UPLC, Mass: Waters Xevo TQ-S Detector (ESI/ESCI), or an Alliance Waters 2695, Mass: Quattromicro™ (ESCI) multimode ionization was employed. Analytical HPLCs were measured e.g. on Alliance Waters 2695). Analytical SFC were performed e.g. on a PIC solution (Software: SFC PIC Lab Online), a WATERS-X⁵ (Software MASSLYNX), or a WATERS-UPC2 (Empower).

Preparative HPLC were performed e.g. on a Waters 2545 (Software Empower), a Gilson (Software Trilution), or a Shimadzu (Software LC Solution). Preparative SFC were performed e.g. on a Waters Thar SFC-80 (Software Chromscope), Waters Thar SFC-150 (Software Chromscope), Waters Thar SFC-200 (Software Chromscope), or a PIC SFC-175 (Software SFC PIC Lab Online).

Structures of example compounds that contain stereocentres are drawn and named with absolute stereochemistry, if known. In case of unknown absolute stereochemistry the compounds can be either racemic, a mixture of diatereomers, a pure diastereomer of unknown stereochemistry, or a pure enantiomer of unknown stereochemistry. Dia 1 and Dia 2 means that diastereiosomers were separated but the stereochemistry is unknown. En 1 and En 2 means that both enantiomers were separated but the absolute configuration is unknown. No suffix given after the compound code means that a compound containing stereocentres was obtained as a racemic mixture or a mixture of diatereomers, respectively, unless the chemical name of the compound specifies the exact stereochemistry.

The LC/MS analysis mentioned in the experimental part were also performed on a Alliance Waters 2695 HPLC (equipped with a PDA detector) connected to a mass spectrometer mass spectrometer Waters Quattromicro (ESCI, multimode ionization). (Method L in the table below).

Conditions used for the HPLC analysis in the experimental part. The LC/MS analysis mentioned in the experimental part were performed on a Alliance Waters 2695 HPLC (equipped with a PDA detector) connected to a mass spectrometer Waters Quattromicro (ESCI, multimode ionization). The separations were performed with a Acquity BEH C18 (1.7 μm, 2.1×50 mm) column and a X-Bridge C18, (3.5 μm, 4.6×75 mm) column thermostated to 30-35° C. and the PDA acquisition wavelength was set in the range of 210-400 nm (Acuisition Software: MassLynx) (Method L in the table below). Elutions were carried out with the methods described in the following tables. For Methods L1 and L6, Solvent A: FA LC-MS grade 0.1% in milliQ water. Solvent B: FA LC-MS grade 0.1% in ACN LC-MS grade. For Methods L2, L3 and L4, Solvent A: FA LC-MS grade 0.05% in milliQ water. Solvent B: FA LC-MS grade 0.05% in ACN LC-MS grade. For Method L5, Solvent A: 5 mM (NH₄)HCO₃ in milliQ water. Solvent B: ACN LC-MS grade.

| HPLC | | Time | Solvents | | Flow | |
|---|---|---|---|---|---|---|
| Method | System | (min) | A (%) | B (%) | (mL/min) | Column |
| L1 | Alliance Waters 2695 | 0 | 97 | 3 | 0.6 | Acquity |
| | HPLC | 0.4 | 97 | 3 | 0.6 | BEH C18 |
| | | 3.2 | 2 | 98 | 0.6 | (0.1% FA in |
| | | 3.8 | 2 | 98 | 0.6 | solvents A |
| | | 4.2 | 97 | 3 | 0.6 | and B) |
| | | 4.5 | 97 | 3 | 0.6 | |
| L2 | Alliance Waters 2695 | 0 | 97 | 3 | 0.6 | Acquity |
| | HPLC | 0.4 | 97 | 3 | 0.6 | BEH C18 |
| | | 3.2 | 2 | 98 | 0.6 | (0.05% FA in |
| | | 3.8 | 2 | 98 | 0.6 | solvents A |
| | | 4.2 | 97 | 3 | 0.6 | and B) |
| | | 4.5 | 97 | 3 | 0.6 | |
| L3 | Alliance Waters 2695 | 0 | 97 | 3 | 0.6 | Acquity |
| | HPLC | 0.4 | 97 | 3 | 0.6 | BEH C18 |
| | | 7.5 | 2 | 98 | 0.6 | (0.05% FA in |
| | | 9.5 | 2 | 98 | 0.6 | solvents A |
| | | 9.6 | 97 | 3 | 0.6 | and B) |
| | | 10 | 97 | 3 | 0.6 | |
| L4 | Alliance Waters 2695 | 0 | 97 | 3 | 0.6 | Acquity |
| | HPLC | 0.4 | 97 | 3 | 0.6 | BEH C18 |
| | | 2.5 | 2 | 98 | 0.6 | (0.05% FA in |
| | | 3.4 | 2 | 98 | 0.6 | solvents A |
| | | 3.5 | 97 | 3 | 0.6 | and B) |
| | | 4 | 97 | 3 | 0.6 | |
| L5 | Alliance Waters 2695 | 0 | 95 | 5 | 1.3 | X-Bridge C18 |
| | HPLC | 0.5 | 95 | 5 | 1.3 | (5 mM |
| | | 1.0 | 85 | 15 | 1.3 | (NH₄)HCO₃ in |
| | | 4.0 | 2 | 98 | 1.3 | solvent A) |
| | | 7.0 | 2 | 98 | 1.3 | |
| | | 7.5 | 95 | 5 | 1.3 | |
| | | 8.0 | 95 | 5 | 1.3 | |
| L6 | Alliance Waters 2695 | 0 | 97 | 3 | 0.6 | Acquity |
| | HPLC | 0.4 | 97 | 3 | 0.6 | BEH C18 |
| | | 2.5 | 2 | 98 | 0.6 | (0.1% FA in |
| | | 3.4 | 2 | 98 | 0.6 | solvents A |
| | | 3.5 | 97 | 3 | 0.6 | and B) |
| | | 4 | 97 | 3 | 0.6 | |

Conditions used for the SF0 analysis in the experimental part. The SF0 analysis mentioned in the experimental part were performed on a WATERS Acquity UPC2 QDa (Empower-3 Sofware) equipped with a Acquity PDA and an Acquity QDa Detector. The separations were performed with a Chiralpak AD-3 (3 μm, 4.6×150 mm), a (R, R) Whelk-01 (3.5 μm, 4.6×150 mm), a Chiralcel OX-3 (3 μm, 4.6×150 mm), or a Chiralpak IG (5 μm, 4.6×150 mm) column; CO₂ as the mobile phase and MeOH as the co-solvent. The column was thermostated at 30° C. Elutions were carried out with the methods described in the following table.

| SCF | |
|---|---|
| Method | Column and conditions |
| S1 | Column: Chiralpak AD-3 (3 μm, 4.6 × 150 mm); % CO₂: 90; co-solvent: 10 |
| | (MeOH); Flow: 3 g/min; ABPR: 1500 psi; Temperature: 30° C. |
| S2 | Column: Chiralpak AD-3 (3 μm, 4.6 × 150 mm); % CO₂: 90; co-solvent: 10 |
| | (0.5% DEA in MeOH); Flow: 3 g/min; ABPR: 1500 psi; Temperature: 30° C. |
| S3 | Column: Chiralpak AD-3 (3 μm, 4.6 × 150 mm); % CO₂: 85; co-solvent: 15 |
| | (MeOH); Flow: 3 g/min; ABPR: 1500 psi; Temperature: 30° C. |
| S4 | Column: Chiralpak AD-3 (3 μm, 4.6 × 150 mm); % CO₂: 85; co-solvent: 15 |
| | (0.5% DEA in EtOH); Flow: 3 g/min; ABPR: 1500 psi; Temperature: 30° C. |
| S5 | Column: Chiralpak AD-3 (3 μm, 4.6 × 150 mm); % CO₂: 80; co-solvent: 20 |
| | (MeOH); Flow: 3 g/min; ABPR: 1500 psi; Temperature: 30° C. |
| S6 | Column: Chiralpak AD-3 (3 μm, 4.6 × 150 mm); % CO₂: 75; co-solvent: 25 |
| | (MeOH); Flow: 3 g/min; ABPR: 1500 psi; Temperature: 30° C. |
| S7 | Column: (R, R) Whelk-O1 (3.5 μm, 4.6 × 150 mm); % CO₂: 80; co-solvent: 20 |
| | (0.5% DEA in MeOH); Flow: 3 g/min; ABPR: 1500 psi; Temperature: 30° C. |
| S8 | Column: Chiralcel OX-3 (3 μm, 4.6 × 150 mm); % CO₂: 70; co-solvent: 30 |
| | (MeOH); Flow: 3 g/min; ABPR: 1500 psi; Temperature: 30° C. |
| S9 | Column: Chiralpak IG (5 μm, 4.6 × 150 mm); % CO₂: 75; co-solvent: 25 |
| | (MeOH); Flow: 3 g/min; ABPR: 1500 psi; Temperature: 30° C. |
| S10 | Column: Chiralpak IG (5 μm, 4.6 × 150 mm); % CO₂: 85; co-solvent: 15 |
| | (0.5% DEA in EtOH); Flow: 3 g/min; ABPR: 1500 psi; Temperature: 30° C. |
| S11 | Column: Chiralpak IG (5 μm, 4.6 × 150 mm); % CO₂: 85; co-solvent: 15 |
| | (MeOH); Flow: 3 g/min; ABPR: 1500 psi; Temperature: 30° C. |

Preparative HPLC purifications mentioned in this experimental part have been carried out with the following system: on a Waters 2545 (Empower software, 2996 PDA detector, 2707 autosampler), a Gilson (Software Trilution, 171 DAD detector, GX-271 autosampler), or a Shimadzu (Software LC Solution, CMB-20A detector, SIL-0AP autosampler). The separations were performed with a Luna 018 (5 μm, 21.2×250 mm), a X-Bridge 018 (5 μm, 29×250 mm or 5 μm, 19×150 mm), a X-Select 018 (5 μm, 19×150 mm or 10 μm, 25×150 mm), a X-Select iSH Phenyl-Hexyl (5 μm, 19×250 mm), a Gemini C18 (5 μm, 30×150 mm), a Kromasil C18 (5 μm, 19×150 mm), a YMC-Triart 018 (10 μm, 19×250 mm), or a Lux Amylose-2 (5 μm, 30×250 mm) column. Elutions were carried out with columns and solvents described in the following table. Gradients systems for each individual compound were employed using the solvents mentioned in the table. Detection wavelengths were fixed at 210 and 254 nm.

| HPLC | |
|---|---|
| Method | Column and conditions |
| H1 | Column: Gemini C18 (5 μm, 30 × 150 mm); Solvent A: 10 mM (NH₄)HCO₃ in water; |
| | Solvent B: ACN/MeOH (70:30); Flow: 16 mL/min. |
| H2 | Luna C18 (5 μm, 21.2 × 250 mm); Solvent A: 10 mM (NH₄)HCO₃ in water; Solvent B: |
| | ACN; Flow: 15 mL/min. |
| H3 | X-Bridge C18 (5 μm, 19 × 150 mm) or X-Bridge C18 (5 μm, 19 × 250 mm); Solvent A: |
| | 10 mM (NH₄)HCO₃ in water; Solvent B: ACN; Flow: 15 mL/min. |
| H4 | X-Select C18 (5 μm, 19 × 150 mm) or X-Select C18 (10 μm, 25 × 250 mm); Solvent A: |
| | 10 mM (NH₄)HCO₃ in water; Solvent B: ACN; Flow: 15 mL/min. |
| H5 | X-Select CSH Phenyl-Hexyl (5 μm, 19 × 250 mm); Solvent A: 10 mM (NH₄)HCO₃ in |
| | water; Solvent B: ACN; Flow: 15 mL/min. |
| H6 | Kromasil C18 (5 μm, 19 × 150 mm); Solvent A: 10 mM (NH₄)HCO₃ in water; Solvent B: |
| | ACN; Flow: 15 mL/min. |
| H7 | X-Select C18 (10 μm, 25 × 150 mm); Solvent A: 10 mM NH₄OAc in water; Solvent B: |
| | ACN; Flow: 17 mL/min. |
| H8 | X-Bridge C18 (5 μm, 19 × 150 mm); Solvent A: 10 mM NH₄OAc in water; Solvent B: |
| | ACN; Flow: 15 mL/min. |
| H9 | X-Select CSH Phenyl-Hexyl (5 μm, 19 × 250 mm); Solvent A: 10 mM NH₄OAc in |
| | water; Solvent B: ACN; Flow: 15 mL/min. |
| H10 | X-Select CSH Phenyl-Hexyl (5 μm, 19 × 250 mm); Solvent A: 0.1% FA in water; |
| | Solvent B: ACN; Flow: 15 mL/min. |
| H11 | Luna C18 (5 μm, 21.2 × 150 mm); Solvent A: 0.1% FA in water; Solvent B: ACN; |
| | Flow: 15 mL/min. |
| H12 | X-Bridge C18 (5 μm, 19 × 150 mm); Solvent A: 0.1% FA in water; Solvent B: ACN; |
| | Flow: 15 mL/min. |
| H13 | X-Select C18 (5 μm, 19 × 150 mm); Solvent A: 0.1% FA in water; Solvent B: ACN; |
| | Flow: 15 mL/min. |
| H14 | YMC-Triart C18 (10 μm, 19 × 250 mm); Solvent A: 10 mM NH₄OAc in water; Solvent B: |
| | ACN:MeOH (1:1); Flow: 22 mL/min. |
| H15 | YMC-Triart C18 (10 μm, 19 × 250 mm); Solvent A: 10 mM (NH4)HCO3 in water; |
| | Solvent B: ACN; Flow: 20 mL/min. |
| H16 | Lux Amylose-2 (5 μm, 30 × 250 mm); Solvent A: n-hexane; Solvent B: 2-propanol; |
| | Flow: 42 mL/min. |

Preparative SF0 purifications mentioned in this experimental part have been carried out with the following system: a Waters Thar SFC-80 or a Thar SCF-200 (Software Chromscope) equipped with a UV/PDA detector and a modifier stream injection mode. The separations were performed with a Chiralpak AD-H (5 μm, 30×250 mm), a (R, R) Whelk-01 (5 μm, 30×250 mm), a Lux Cellulose-4 (5 μm, 30×250 mm), or a Lux i-Amylose-3 (5 μm, 30×250 mm) column; CO₂ as the mobile phase and MeOH as the co-solvent. The column was thermostated at 30° C. Detection wavelengths were fixed at 214 nm. Elutions were carried out with the methods described in the following table.

| Prep | |
|---|---|
| SFC | |
| Method | Column and conditions |
| K1 | Chiralpak AD-H (5 μm, 30 × 250 mm); % CO₂: 85; % co-solvent: 15 (MeOH); Flow: |
| | 100 g/min; ABPR: 1500 psi; Temperature: 30° C. |
| K2 | Chiralpak AD-H (5 μm, 30 × 250 mm); % CO₂: 85; % co-solvent: 15 (15 mM NH₃ in |
| | MeOH); Flow: 60 g/min; ABPR: 1500 psi; Temperature: 30° C. |
| K3 | Chiralpak AD-H (5 μm, 30 × 250 mm); % CO₂: 90; % co-solvent: 10 (0.5% DEA in |
| | MeOH); Flow: 100 g/min; ABPR: 1500 psi; Temperature: 30° C. |
| K4 | Chiralpak AD-H (5 μm, 30 × 250 mm); % CO₂: 80; % co-solvent: 20 (EtOH); Flow: |
| | 60 g/min; ABPR: 1500 psi; Temperature: 30° C. |
| K5 | Chiralpak AD-H (5 μm, 30 × 250 mm); % CO₂: 80; % co-solvent: 20 (MeOH); Flow: |
| | 100 g/min; ABPR: 1500 psi; Temperature: 30° C. |
| K6 | Chiralpak AD-H (5 μm, 30 × 250 mm); % CO₂: 70; % co-solvent: 30 (MeOH); Flow: |
| | 100 g/min; ABPR: 1500 psi; Temperature: 30° C. |
| K7 | (R,R) Whelk-O1 (5 μm, 30 × 250 mm); % CO₂: 80; % co-solvent: 20 (30 mM NH₃ in |
| | MeOH); Flow: 90 g/min; ABPR: 1500 psi; Temperature: 30° C. |
| K8 | Lux Cellulose-4 (5 μm, 30 × 250 mm); % CO₂: 60; % co-solvent: 40 (MeOH); Flow: |
| | 60 g/min; ABPR: 1500 psi; Temperature: 30° C. |
| K9 | Lux i-Amylose-3 (5 μm, 30 × 250 mm); % CO₂: 80; % co-solvent: 20 (30 mM NH₃ in |
| | MeOH); Flow: 100 g/min; ABPR: 1700 psi; Temperature: 30° C. |
| K10 | Chiralpak AD-H (5 μm, 30 × 250 mm); % CO₂: 85; % co-solvent: 15 (EtOH); Flow: |
| | 90 g/min; ABPR: 1500 psi; Temperature: 30° C. |

### Synthesis of tert-butyl (2-oxo-1,2,3,4-tetrahydroquinolin-3-yl)carbamate (Int-1)

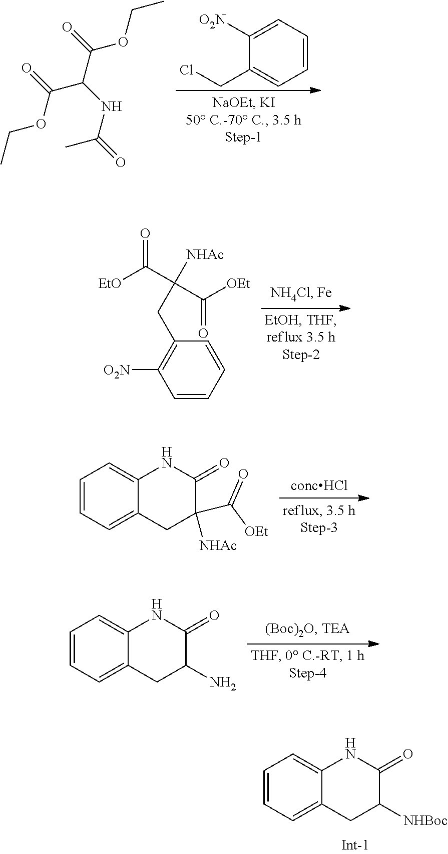 [SMILES: CCOC(=O)C(NC(C)=O)C(=O)OCC ; O=[N+]([O-])c1ccccc1CCl ; CCOC(=O)C(Cc1ccccc1[N+](=O)[O-])(NC(C)=O)C(=O)OCC ; CCOC(=O)C1(NC(C)=O)Cc2ccccc2NC1=O ; NC1Cc2ccccc2NC1=O ; CC(C)(C)OC(=O)NC1Cc2ccccc2NC1=O]

Step 1: Diethyl 2-acetamidomalonate (11.0 g, 50.6 mmol) was treated with a solution of freshly prepared NaOEt solution (1.2 g of Na metal was dissolved in in 50 mL EtOH at 0° C. under a nitrogen atmosphere) at RT and stirred at 50° C. for 1 h. The reaction mixture was treated with 1-(chloromethyl)-2-nitrobenzene (8.6 g, 50.6 mmol) and KI (0.4 g, 2.5 mmol) at 50° C. and stirred at 70° C. for 2.5 h. Progress of the reaction was monitored by TLC. TLC mobile phase: 30% EtOAc in pet ether, RF: 0.2, TLC detection: UV. The reaction mixture was diluted with ice water (100 mL) and the product was extracted with EtOAc (3×70 mL). The organic layer was dried over Na₂SO₄ and evaporated under reduced pressure to afford crude product (13.0 g, LC/MS 79%) which was purified by flash chromatography (grace) using a 80 g reveler is column and a gradient of 50% EtOAc in pet ether to afford diethyl 2-acetamido-2-(2-nitrobenzyl)malonate as a pale yellow solid (10 g, 56%). (LC/MS; m/z 352.9 [M+H]⁺) Step 2: A sat. NH₄Cl solution (20 mL) and iron powder (6.3 g, 113.3 mmol) were added to a stirred solution of diethyl 2-acetamido-2-(2-nitrobenzyl)malonate (10.0 g, 28.4 mmol) in EtOH (50 mL) and THF (25 mL) at RT. The reaction mixture was heated to reflux (90° C.) for 3.5 h. Progress of the reaction was monitored by TLC. TLC mobile phase: 50% EtOAc in pet ether, RF:0.5, TLC detection: UV. The reaction mixture was filtered through a celite pad and washed several times with MeOH (100 mL). The filtrate was concentrated and diluted with EtOAc (100 mL), washed with water (2×100 mL), dried over Na₂SO₄ and concentrated under reduced pressure to afford ethyl 3-acetamido-2-oxo-1,2,3,4-tetrahydroquinoline-3-carboxylate as a beige solid (7.0 g, 89%). (LC/MS; m/z 277.1 [M+H]⁺)

Step 3: Ethyl 3-acetamido-2-oxo-1,2,3,4-tetrahydroquinoline-3-carboxylate (8 g, 28.98 mmol) was dissolved in conc. HCl (40 mL) and heated (110° C.) to reflux for 3.5 h. Progress of the reaction was monitored by TLC. TLC mobile phase: 50% EtOAc in pet ether. The reaction mixture was cooled to RT, diluted with water (50 mL) and extracted into 10% MeOH in DCM (2×50 mL). The organic fractions were discarded and the aq. phase was basified with aq. NaOH solution (pH>12) and extracted into 10% MeOH in DCM (3×40 mL). The organic fractions were washed with brine (100 mL), dried over Na₂SO₄ and concentrated under reduced pressure to afford 3-amino-3,4-dihydroquinolin-2(1H)-one an off-white solid (1.5 g, 31%). (LC/MS; m/z 161.0 [M+H]⁺). Along with this compound, 3 g of N-acetylated intermediate was isolated. (LC/MS; m/z 205.1 [M+H]⁺) Step 4: A solution of 3-amino-3,4-dihydroquinolin-2(1H)-one (2.0 g, 12.3 mmol) in THF (16 mL) was cooled to 0° C., treated with TEA (3.5 mL, 24.6 mmol) followed by a solution of (Boc)₂O (2.6 mL, 12.3 mmol) in THF (4 mL) under an argon atmosphere. The solution was stirred at RT for 1 h. The reaction was monitored by TLC. TLC mobile phase: 50% EtOAc in pet ether, RF: 0.53, TLC detection: UV. The reaction mixture was diluted with cold water (20 mL) and extracted with EtOAc (20 mL). The organic layer was separated, dried over Na₂SO₄ and evaporated under reduced pressure to afford crude product (1.9 g) which was purified by flash chromatography (Grace) using a 40 g reveleris column and 50% EtOAc in pet ether gradient to afford tert-butyl (2-oxo-1,2,3,4-tetrahydroquinolin-3-yl)carbamate (Int-1) as an off-white solid (1.5 g, 46%). (LC/MS; m/z 263.0 [M+H]⁺)

### Example 1: Synthesis of N-(1-(4-(trifluoromethyl)phenyl)-1,2,3,4-tetrahydroquinolin-3-yl)acrylamide (Cpd. No. 001)

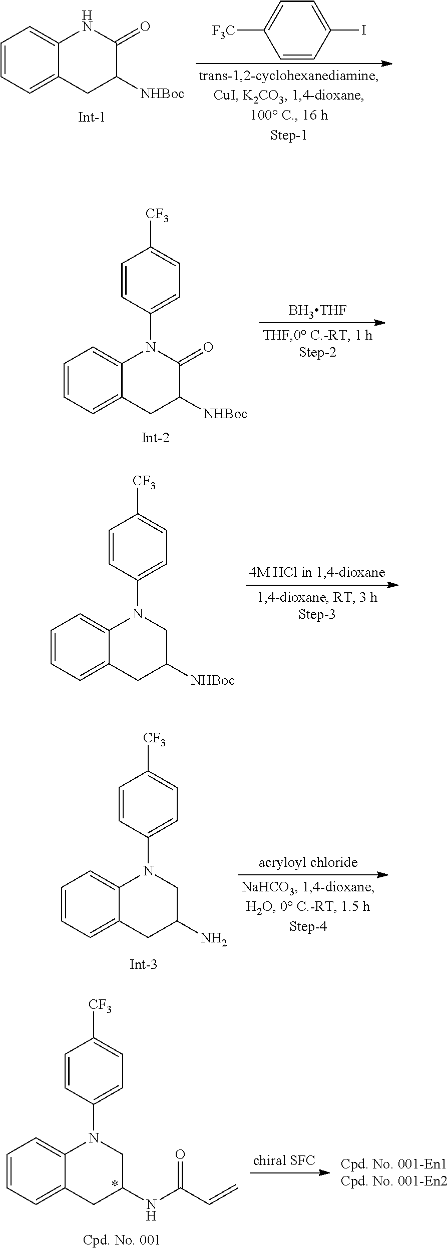

Step 1: A solution of tert-butyl (2-oxo-1,2,3,4-tetrahydroquinolin-3-yl)carbamate (Int-1) (2.1 g, 8 mmol) in 1,4-dioxane (20 mL) was treated with 1-iodo-4-(trifluoromethyl)benzene (4.3 g, 16 mmol), trans-1,2-cyclohexanediamine (182 mg, 1.62 mmol), Cul (304 mg, 1.6 mmol) and K₂CO₃ (2.42 g, 17.6 mmol) under argon at RT. The reaction mixture was stirred at 100° C. for 16 h (sealed tube). Progress of the reaction was monitored by TLC. TLC mobile phase: 30% EtOAc in pet ether. The reaction mixture was filtered through a celite pad and washed with EtOAc (30 mL). The filtrate was evaporated under reduced pressure to afford crude product (2.5 g) which was purified by flash chromatography (Grace) using a 40 g reveleris column and 50% EtOAc in pet ether gradient to afford tert-butyl (2-oxo-1-(4-(trifluoromethyl)phenyl)-1,2,3,4-tetrahydroquinolin-3-yl)carbamate (Int-2) as an off-white solid (1.3 g, 40%). (LC/MS; m/z 407.2 [M+H]⁺)

Step 2: A solution of tert-butyl (2-oxo-1,2,3,4-tetrahydroquinolin-3-yl)carbamate (Int-2) (1.5 g, 3.7 mmol) in THF (15 mL) was cooled to 0° C., treated with BH₃·THF (1M in THF, 18.0 mL, 18.5 mmol) under a nitrogen atmosphere. The reaction mixture was stirred at RT for 1 h. Progress of the reaction was monitored by TLC. TLC mobile phase: 10% EtOAc in pet ether, RF: 0.5, TLC detection: UV. The reaction mixture was cooled to 0° C., quenched with MeOH (13 mL) and evaporated under reduced pressure to afford crude product (1.4 g) which was purified by flash chromatography (Grace) using a 40 g reveleris column and eluted with 40% EtOAc in pet ether gradient to afford tert-butyl (1-(4-(trifluoromethyl) phenyl)-1,2,3,4-tetrahydroquinolin-3-yl)carbamate (1.1 g, 76%). (LC/MS; m/z 393.0 [M+H]⁺)

Step 3: A solution of tert-butyl (1-(4-(trifluoromethyl)phenyl)-1,2,3,4-tetrahydroquinolin-3-yl)carbamate (1.1 g, 2.8 mmol) in 1,4-dioxane (5 mL) was treated with HCl (4M in 1,4-dioxane) (10 mL) at 0° C. under a nitrogen atmosphere. The reaction mixture was stirred at RT for 3 h. Progress of the reaction was monitored by TLC. TLC mobile phase: 20% EtOAc in pet ether. The reaction mixture was evaporated under reduced pressure, the obtained residue was washed with Et₂O (12 mL) and dried to afford a yellow solid (850 mg, 92%). 140 mg of 1-(4-(trifluoromethyl)phenyl)-1,2,3,4-tetrahydroquinolin-3-amine hydrochloride (Int-3·HCl) was further purified by preparative HPLC method H2. The collected fractions were lyophilised to afford 1-(4-(trifluoromethyl)phenyl)-1,2,3,4-tetrahydroquinolin-3-amine (Int-3; Cpd. No. 171) as an off-white solid (20 mg, 21%) (LC/MS; m/z 293.2 [M+H]⁺)

Step 4: A solution of 1-(4-(trifluoromethyl)phenyl)-1,2,3,4-tetrahydroquinolin-3-amine hydrochloride (Int-3·HCl) (100 mg, 0.304 mmol) in 1,4-dioxane (2 mL) and water (1 mL) was cooled to 0° C., treated with NaHCO₃ (128 mg, 1.52 mmol) and acryloyl chloride (33 mg, 0.36 mmol). The reaction mixture was stirred for 1.5 h at RT. Progress of the reaction was monitored by TLC. TLC mobile phase: 50% EtOAc in pet ether, RF: 0.50, TLC detection: UV. The reaction mixture was diluted with cold water (8 mL) and extracted with EtOAc (10 mL). The organic layer was separated, dried over Na₂SO₄ and evaporated under reduced pressure to afford crude product (90 mg) which was combined with additional 80 mg of crude product from another reaction batch and purified by preparative HPLC method H4. Collected fractions were lyophilised to afford N-(1-(4-(trifluoromethyl)phenyl)-1,2,3,4-tetrahydroquinolin-3-yl)acrylamide (Cpd. No. 001) as an off-white solid (20 mg, 10%). (LC/MS; m/z 347.2 [M+H]⁺). Chiral SFC purification: 60 mg of Cpd. No. 001 was further purified by preparative SFC method K1 to afford Cpd. No. 001-En1 (10 mg) and Cpd. No. 001-En2 (10 mg), both as an off-white solid. (LC/MS; m/z 347.2 [M+H]⁺). The chiral purity of both enantiomers was assessed by analytic SFC method S1: Cpd. No. 001-En1, 99.9% ee; Cpd. No. 001-En2, 99.2% ee.

The following compounds were prepared in a manner similar to Cpd. No. 001, by using appropriate reagents and purification methods known to the person skilled in the art: Cpd. No. 002, Cpd. No. 003, Cpd. No. 004, Cpd. No. 005, Cpd. No. 006, Cpd. No. 007, Cpd. No. 008, Cpd. No. 009, Cpd. No. 010, Cpd. No. 011, and Cpd. No. 012.

### Example 2: Synthesis of N-(1-cyclohexyl-1,2,3,4-tetrahydroquinolin-3-yl)acrylamide (Cpd. No. 013)

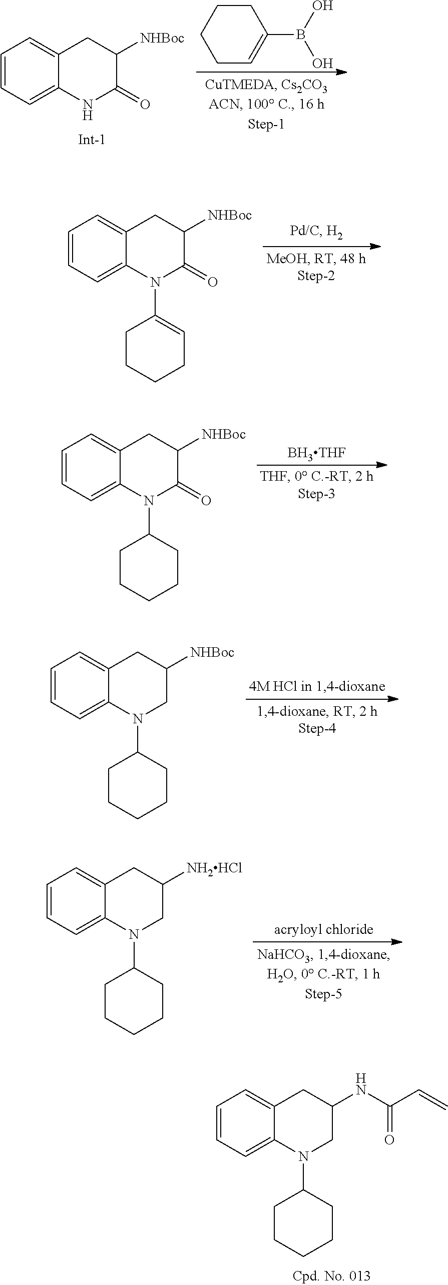

Step 1: A solution of cyclohex-1-en-1-ylboronic acid (1.9 g, 15.2 mmol) in ACN (10 mL) was treated with CuTMEDA (2.1 g, 4.5 mmol), Cs₂CO₃ (2.47 g, 7.6 mmol) and tert-butyl (2-oxo-1,2,3,4-tetrahydroquinolin-3-yl)carbamate (Int-1) (1 g, 3.8 mmol) and stirred at 100° C. for 16 h (sealed tube). Progress of the reaction was monitored by TLC. TLC mobile phase: 20% EtOAc in pet ether. RF: 0.6, TLC detection: UV. The reaction mixture was cooled to RT, filtered through a celite pad and rinsed with EtOAc (50 mL). The filtrate was washed with water (30 mL) and brine (30 mL). The organic layer was dried over anhydrous Na₂SO₄, filtered and filtrate was concentrated under reduced pressure to afford crude product as a pale brown gum (1.1 g). Which was purified by normal phase chromatography (Grace) using silica (100-200 mesh) and a gradient of 14% EtOAc in pet ether. The collected fractions were concentrated under reduced pressure to afford tert-butyl (1-(cyclohex-1-en-1-yl)-2-oxo-1,2,3,4-tetrahydroquinolin-3-yl)carbamate as an off-white solid (140 mg, 5%). (LC/MS; m/z 343.3 [M+H]⁺)

Step 2: A solution of tert-butyl (1-(cyclohex-1-en-1-yl)-2-oxo-1,2,3,4-tetrahydroquinolin-3-yl)carbamate (140 mg, 0.40 mmol, LC/MS 83%) in MeOH (5 mL) was treated with 10% Pd/C (45 mg) under a nitrogen atmosphere and stirred at RT under a hydrogen atmosphere (balloon pressure) for 48 h. Progress of the reaction was monitored by TLC. TLC mobile phase: 10% EtOAc in pet ether. RF: 0.2, TLC detection: UV. The reaction mixture was filtered through a celite pad and rinsed with MeOH (20 mL). The filtrate was concentrated under reduced pressure to afford tert-butyl (1-cyclohexyl-2-oxo-1,2,3,4-tetrahydroquinolin-3-yl)carbamate as an off-white gum (62 mg, 44%). (LC/MS; m/z 345.4 [M+H]⁺)

Steps 3-5: These steps were executed in a manner similar (use of appropriate reagents and purification methods known to the person skilled in the art) to Cpd. No. 001. Starting material (70 mg, 0.21 mmol, LC/MS 45%) yielded a pale brown gum which was purified preparative HPLC method H3. The obtained fraction was lyophilised to afford N-(1-cyclohexyl-1,2,3,4-tetrahydroquinolin-3-yl)acrylamide (Cpd. No. 013) as an off-white solid (5 mg, 19%). (LC/MS; m/z 285.2 [M+H]⁺)

### Example 3: Synthesis of 4-(3-acrylamido-3,4-dihydroquinolin-1(2H)-yl)—N-methylbenzamide (Cpd. No. 014)

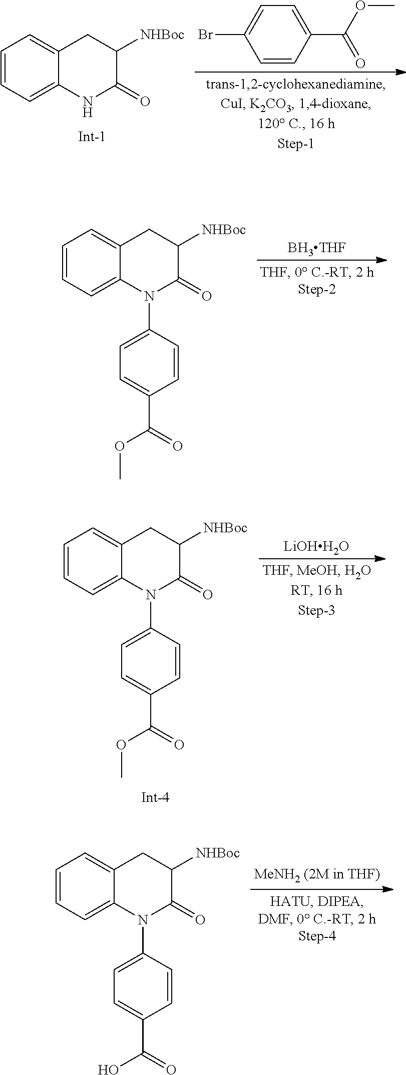

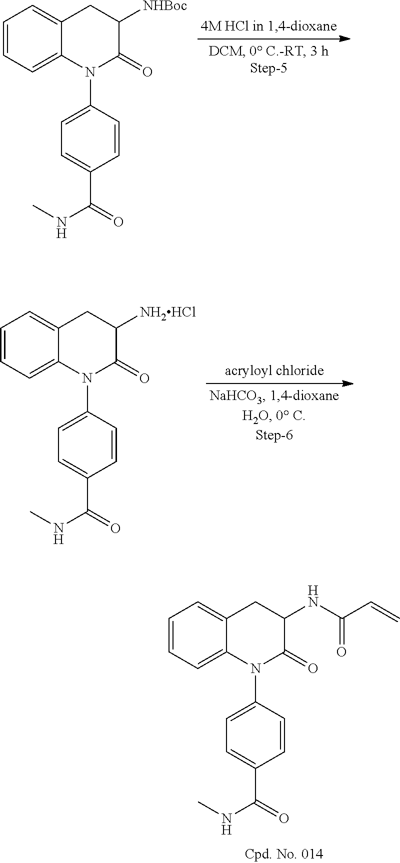

Steps 1-2: These steps were executed in a manner similar (use of appropriate reagents and purification methods known to the person skilled in the art) to Cpd. No. 001. Starting material Int-1 (500 mg, 1.90 mmol) yielded methyl 4-(3-((tert-butoxycarbonyl)amino)-3,4-dihydroquinolin-1(2H)-yl)benzoate (Int-4) as a white solid (350 mg, 72%). (LC/MS; m/z 383.3 [M+H]⁺)

Step 3: A solution of methyl 4-(3-((tert-butoxycarbonyl)amino)-3,4-dihydroquinolin-1(2H)-yl)benzoate (Int-4) (250 mg, 0.65 mmol, LC/MS 83%) in THF (2 mL), MeOH (2 mL) and water (2 mL) was treated with LiOH·H₂O (107 mg, 2.61 mmol) at 0° C. and the reaction mixture was stirred at RT for 16 h. The reaction progress was monitored by TLC. TLC mobile phase: 30% EtOAc in pet ether, RF: 0.1, TLC detection: UV. The reaction mixture was concentrated and diluted with water (5 mL), acidified with 1N HCl (pH 6) and extracted with EtOAc (2×10 mL). The organic layer was dried over Na₂SO₄ and concentrated to afford 4-(3-((tert-butoxycarbonyl)amino)-3,4-dihydroquinolin-1(2H)-yl)benzoic acid as a pale yellow solid (210 mg, 93%). (LC/MS; m/z 369.0 [M+H]⁺)

Step 4: A solution of 4-(3-((tert-butoxycarbonyl)amino)-3,4-dihydroquinolin-1(2H)-yl)benzoic acid (250 mg, 0.67 mmol, LC/MS 89%) in DMF (3 mL) was treated with HATU (516 mg, 1.35 mmol), DIPEA (219 mg, 1.69 mmol), methyl amine solution (2M in THF, 1.3 mL, 2.68 mmol) at 0° C. under a nitrogen atmosphere and stirred at RT for 2 h. The reaction progress was monitored by TLC. TLC mobile phase: 50% EtOAc in pet ether, RF: 0.20, TLC detection: UV. The reaction mixture was diluted with EtOAc (20 mL) and ice water (20 mL). The organic layer was separated, washed with ice water (4×20 mL), dried over Na₂SO₄ and concentrated to afford tert-butyl (1-(4-(methylcarbamoyl)phenyl)-1,2,3,4-tetrahydroquinolin-3-yl)carbamate as a pale yellow gum (260 mg, 78%). (LC/MS; m/z 382.1 [M+H]⁺)

Steps 5-6: These steps were executed in a manner similar (use of appropriate reagents and purification methods known to the person skilled in the art) to Cpd. No. 001. Starting material (280 mg, 0.73 mmol, LC/MS 70%) yielded a pale yellow solid which was purified preparative HPLC method H4. The collected fractions were concentrated and lyophilised under vacuum to afford 4-(3-acrylamido-3,4-dihydroquinolin-1(2H)-yl)—N-methylbenzamide (Cpd. No. 014) as an off-white solid (41 mg, 43%). (LC/MS; m/z 336.2 [M+H]⁺)

### Synthesis of tert-butyl (1,2,3,4-tetrahydroquinolin-3-yl)carbamate (Int-5)

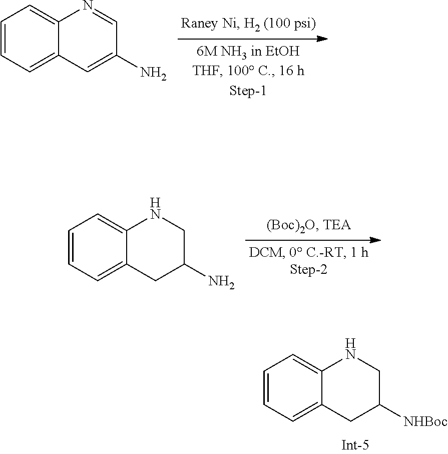 [SMILES: Nc1cnc2ccccc2c1 ; NC1CNc2ccccc2C1 ; CC(C)(C)OC(=O)NC1CNc2ccccc2C1]

Step 1: A solution of quinolin-3-amine (1.0 g, 6.9 mmol) in THF (10 mL) (steal bomb) was treated with 6M NH₃ in EtOH (3 mL), Raney Ni (2.0 g) under a nitrogen atmosphere. The reaction mixture was stirred under hydrogen gas (100 psi) and heated to 100° C. for 16 h. The reaction progress was monitored by TLC. TLC mobile phase: 10% MeOH in DCM, RF: 0.20, TLC detection: UV. The reaction mixture was filtered through a celite pad and washed with MeOH (20 mL). The filtrate was evaporated under reduced pressure to afford crude 1,2,3,4-tetrahydroquinolin-3-amine as a brown gum (600 mg, 48%). (LC/MS; m/z 149.1 [M+H]⁺)

Step 2: A solution 1,2,3,4-tetrahydroquinolin-3-amine (600 mg, 4.05 mmol, LC/MS 82.78%) in DCM (20 mL) was cooled to 0° C., treated with TEA (410 mg, 4.05 mmol), (Boc)₂O (795 mg, 3.646 mmol) under a nitrogen atmosphere and stirred at RT for 1 h. The reaction progress was monitored by TLC. TLC mobile phase: 20% EtOAc in pet ether, RF: 0.5, TLC detection: UV. The reaction mixture was diluted with water (20 mL) and the product was extracted with DCM (2×50 mL). The organic layer was separated, dried over Na₂SO₄ and evaporated under reduced pressure to afford tert-butyl (1,2,3,4-tetrahydroquinolin-3-yl)carbamate (Int-5) as a pale brown solid (600 mg, 59%). (LC/MS; m/z 249.2 [M+H]⁺)

### Example 4: Synthesis of N-(1-(3-acetamidophenyl)-1,2,3,4-tetrahydroquinolin-3-yl)acrylamide (Cpd. No. 015)

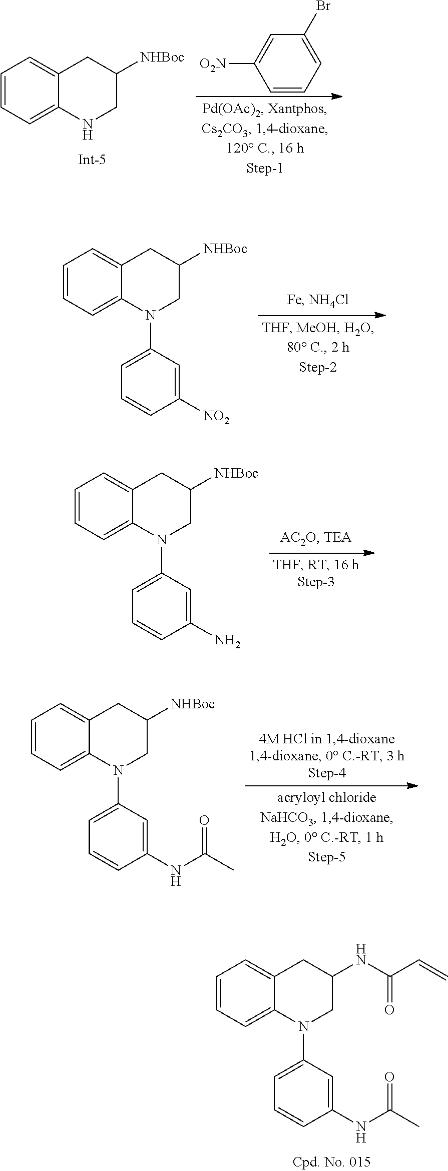 [SMILES: CC(C)(C)OC(=O)NC1CNc2ccccc2C1 ; O=[N+]([O-])c1cccc(Br)c1 ; CC(C)(C)OC(=O)NC1Cc2ccccc2N(c2cccc([N+](=O)[O-])c2)C1 ; CC(C)(C)OC(=O)NC1Cc2ccccc2N(c2cccc(N)c2)C1 ; CC(=O)Nc1cccc(N2CC(NC(=O)OC(C)(C)C)Cc3ccccc32)c1 ; C=CC(=O)NC1Cc2ccccc2N(c2cccc(NC(C)=O)c2)C1]

Step 1: A solution of 1-bromo-3-nitrobenzene (100 mg, 0.49 mmol) and tert-butyl (1,2,3,4-tetrahydroquinolin-3-yl)carbamate (Int-5) (172 mg, 0.69 mmol) in 1,4-dioxane (10 mL) was treated with Cs₂CO₃ (323 mg, 0.99 mmol), Pd(OAc)₂ (14 mg, 0.062 mmol) and Xantphos (34 mg, 0.058 mmol) at RT. The reaction mixture was stirred in a sealed tube at 120° C. for 16 h. The reaction progress was monitored by TLC. TLC mobile phase: 20% EtOAc in pet ether, RF: 0.3, TLC detection: UV. The reaction mixture was diluted with EtOAc (50 mL) and washed with brine (3×50 mL). The EtOAc layer was dried over anhydrous Na₂SO₄, filtered and the filtrate was concentrated under reduced pressure to afford crude product (250 mg, LC/MS purity 70%) as a brown gum, which was purified by column chromatography using silica gel (100-200 mesh, 10 g). The product was eluted with 15% EtOAc in pet ether. The obtained fractions were concentrated under reduced pressure to afford tert-butyl (1-(3-nitrophenyl)-1,2,3,4-tetrahydroquinolin-3-yl)carbamate as an off-white solid (180 mg, 99%). (LC/MS; m/z 370.3 [M+H]⁺)

Step 2: A solution of tert-butyl (1-(3-nitrophenyl)-1,2,3,4-tetrahydroquinolin-3-yl)carbamate (135 mg, 0.36 mmol, LC/MS 95%) in THF (4 mL), MeOH (2 mL) and water (1 mL) was treated with Fe (201 mg, 3.5 mmol) and NH₄Cl (388 mg, 7.18 mmol) at RT. The reaction mixture was stirred at 80° C. for 2 h. The reaction progress was monitored by TLC. TLC mobile phase: 50% EtOAc in pet ether. RF: 0.2, TLC detection: UV. The reaction mixture was diluted with EtOAc (50 mL) and washed with brine (2×40 mL). The EtOAc layer was dried over anhydrous Na₂SO₄, filtered and filtrate was concentrated under reduced pressure to afford tert-butyl (1-(3-aminophenyl)-1,2,3,4-tetrahydroquinolin-3-yl)carbamate as a yellow gum (85 mg, 69%). (LC/MS; m/z 340.1 [M+H]⁺)

Step 3: A solution of tert-butyl (1-(3-aminophenyl)-1,2,3,4-tetrahydroquinolin-3-yl)carbamate (85 mg, 0.25 mmol, LC/MS 87%) in THF (5 mL) was treated with TEA (126 mg, 1.2 mmol) and AC₂O (31 mg, 0.3 mmol) at 0° C. The reaction mixture was stirred at RT for 16 h. The reaction progress was monitored by TLC. TLC mobile phase: 80% EtOAc in pet ether. RF: 0.2, TLC detection: UV.

The reaction mixture was diluted with EtOAc (150 mL) and washed with brine (2×100 mL). The EtOAc layer was dried over anhydrous Na₂SO₄, filtered and filtrate was concentrated under reduced pressure to afford crude product (130 mg) as a yellow gum, which was purified by column chromatography using silica gel (100-200 mesh, 12 g). The product was eluted with 70% EtOAc in pet ether. The obtained fractions were concentrated under reduced pressure to afford tert-butyl (1-(3-acetamidophenyl)-1,2,3,4-tetrahydroquinolin-3-yl)carbamate as a yellow gum (75 mg, 79%). (LC/MS; m/z 382.1 [M+H]⁺)

Step 4-5: These steps were executed in a manner similar (use of appropriate reagents and purification methods known to the person skilled in the art) to Cpd. No. 001. Starting material (72 mg, 0.18 mmol, LC/MS 96%) yielded a brown gum which was purified by preparative HPLC method H2. The collected fractions were concentrated under reduced pressure and lyophilised to afford N-(1-(3-acetamidophenyl)-1,2,3,4-tetrahydroquinolin-3-yl)acrylamide (Cpd. No. 015) as an off-white solid (10 mg, 20%). (LC/MS; m/z 336.3 [M+H]⁺)

### Synthesis of tert-butyl 5-bromo-1H-indole-1-carboxylate (Int-6)

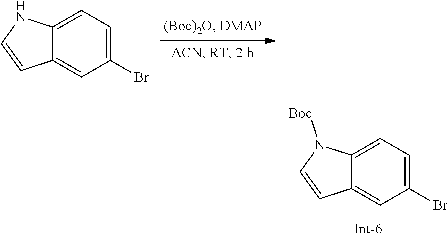 [SMILES: Brc1ccc2[nH]ccc2c1 ; CC(C)(C)OC(=O)n1ccc2cc(Br)ccc21]

A solution of 5-bromo-1H-indole (1 g, 5.1 mmol) in ACN (10 mL) was treated with DMAP (0.031 g, 0.25 mmol) and (Boc)₂O (1.4 g, 6.63 mmol) at RT under a nitrogen atmosphere. The reaction mixture was stirred at RT for 2 h. progress of the reaction was monitored by TLC. TLC mobile phase: 20% EtOAc in pet ether, RF: 0.63, TLC detection: UV. The reaction mixture was diluted with water (80 mL) and extracted with EtOAc (2×100 mL). The combined organic layer was dried over Na₂SO₄, filtered and concentrated under reduced pressure to afford tert-butyl 5-bromo-1H-indole-1-carboxylate (Int-6) as a pale brown solid (1.2 g, 79%). (LC/MS; m/z 296.1 [M+H]⁺)

The following intermediate (Int-7) was prepared in a manner similar (use of appropriate reagents and purification methods known to the person skilled in the art) to Int-6:

| Cpd. | | [M + H]⁺ |
|---|---|---|
| Nr. | Structure | (m/z) |
| Int-7 | 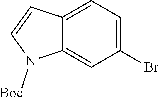 [SMILES: CC(C)(C)OC(=O)n1ccc2ccc(Br)cc21] | 296.1 |

### Example 5: Synthesis of N-(1-(1H-indol-5-yl)-1,2,3,4-tetrahydroquinolin-3-yl)acrylamide (Cpd. No. 016)

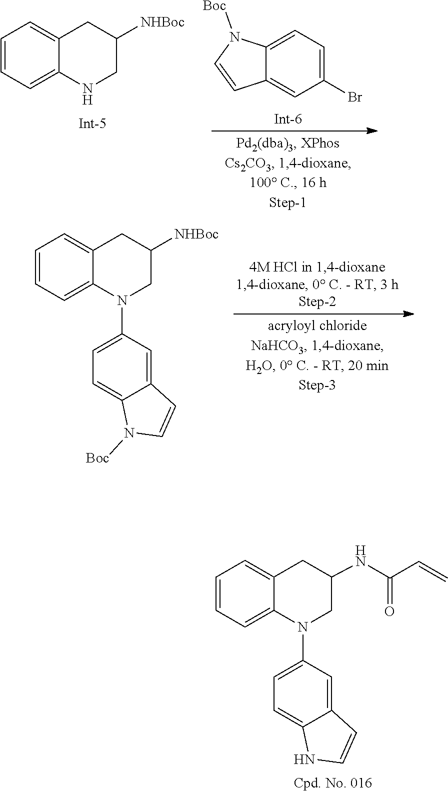 [SMILES: CC(C)(C)OC(=O)NC1CNc2ccccc2C1 ; CC(C)(C)OC(=O)n1ccc2cc(Br)ccc21 ; CC(C)(C)OC(=O)NC1Cc2ccccc2N(c2ccc3c(ccn3C(=O)OC(C)(C)C)c2)C1 ; C=CC(=O)NC1Cc2ccccc2N(c2ccc3[nH]ccc3c2)C1]

Step 1: A solution of tert-butyl (1,2,3,4-tetrahydroquinolin-3-yl)carbamate (Int-5) (400 mg, 1.61 mmol, LC/MS 98%) in 1,4-dioxane (10 mL) (sealed tube) was treated with tert-butyl 5-bromo-1H-indole-1-carboxylate (Int-6) (951 mg, 3.22 mmol, LC/MS 99%), Cs₂CO₃ (1051 mg, 3.22 mmol) at RT and degassed with argon for 5 min. XPhos (153 mg, 0.32 mmol) and Pd₂(dba)₃ (147 mg, 0.16 mmol) were added to the reaction mixture and stirred at 100° C. for 16 h. Progress of the reaction was monitored by TLC. TLC mobile phase: 20% EtOAc in pet ether, RF: 0.55, TLC detection: UV. The reaction mixture was cooled to RT, diluted with water (50 mL) and extracted with EtOAc (2×100 mL). The combined organic layer was dried over Na₂SO₄, filtered and concentrated under reduced pressure to afford crude product as a yellow gum (500 mg, LC/MS 38%) which was purified by normal phase column chromatography (Grace) using 24 g column and a gradient of 10% EtOAc in pet ether as an eluent to afford tert-butyl 5-(3-((tert-butoxycarbonyl)amino)-3,4-dihydroquinolin-1(2H)-yl)-1H-indole-1-carboxylate as a yellow solid (300 mg, 35%). (LC/MS; m/z 464.1 [M+H]⁺)

Step 2-3: These steps were executed in a manner similar (use of appropriate reagents and purification methods known to the person skilled in the art) to Cpd. No. 001. Starting material (300 mg, 0.64 mmol, LC/MS 87%) yielded a pale yellow gum which was purified by preparative HPLC method H2. The collected fractions were concentrated under reduced pressure and lyophilised to afford N-(1-(1H-indol-5-yl)-1,2,3,4-tetrahydroquinolin-3-yl)acrylamide (Cpd. No. 016) as an off-white solid (12 mg, 17%). (LC/MS; m/z 318.2 [M+H]⁺)

The following compounds were prepared in a manner similar to Cpd. No. 016 by using appropriate reagents and purification methods known to the person skilled in the art: Cpd. No. 017, Cpd. No. 018, Cpd. No. 019, Cpd. No. 020, Cpd. No. 021, Cpd. No. 022, Cpd. No. 023, Cpd. No. 024, Cpd. No. 025, Cpd. No. 026, Cpd. No. 027, Cpd. No. 028, Cpd. No. 029, Cpd. No. 030, Cpd. No. 031, and Cpd. No. 142. Int-7 was utilized to prepare compound Cpd. No. 017.

### Example 6: Synthesis of N-(1-(3-chlorophenyl)-1,2,3,4-tetrahydroquinolin-3-yl)acrylamide (Cpd. No. 032)

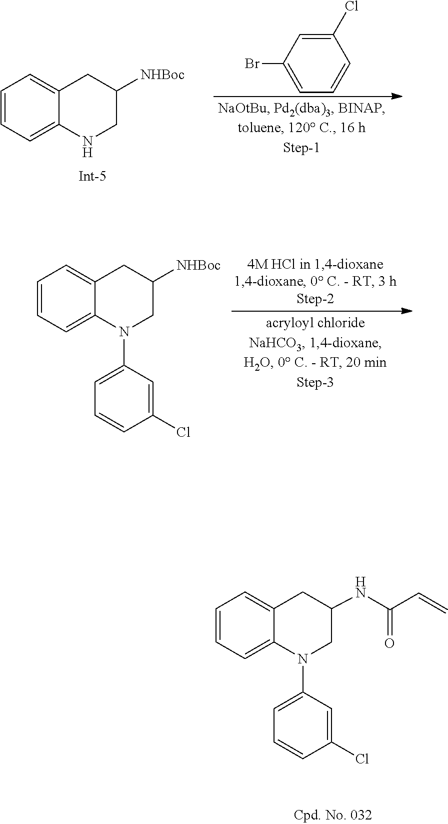 [SMILES: CC(C)(C)OC(=O)NC1CNc2ccccc2C1 ; Clc1cccc(Br)c1 ; CC(C)(C)OC(=O)NC1Cc2ccccc2N(c2cccc(Cl)c2)C1 ; C=CC(=O)NC1Cc2ccccc2N(c2cccc(Cl)c2)C1]

Step 1: A solution of tert-butyl (1,2,3,4-tetrahydroquinolin-3-yl)carbamate (Int-5) (100 mg, 0.52 mmol), 1-bromo-3-chlorobenzene (195 mg, 0.78 mmol) and NaOtBu (93 mg, 0.97 mmol in toluene (10 mL) (sealed tube) was degassed with argon for 3 min. BINAP (26 mg, 41 μmol) and Pd₂(dba)₃ (19 mg, 20 μmol) were added to the reaction mixture and stirred at 120° C. for 16 h. The reaction progress was monitored by TLC. TLC mobile phase: 20% EtOAc in pet ether, RF: 0.6, TLC detection: UV. The reaction mixture was concentrated under reduced pressure to afford crude (400 mg, LC/MS 39%) which was purified by normal phase chromatography (Grace) using a 24 g reveleris column and a gradient of 12% EtOAc in pet ether as an eluent to afford tert-butyl (1-(3-chlorophenyl)-1,2,3,4-tetrahydroquinolin-3-yl)carbamate as an off-white solid (75 mg, 40%). (LC/MS; m/z 359.0 [M+H]⁺)

Steps 2-3: These steps were executed in a manner similar (use of appropriate reagents and purification methods known to the person skilled in the art) to Cpd. No. 001. Starting material (130 mg, 0.36 mmol) yielded crude product which was purified by preparative HPLC method H3. The collected fractions were concentrated under reduced pressure and lyophilised to afford N-(1-(3-chlorophenyl)-1,2,3,4-tetrahydroquinolin-3-yl)acrylamide (Cpd. No. 032) as an off-white solid (37 mg, 42%). (LC/MS; m/z 313.2 [M+H]⁺)

### Example 7: Synthesis of N-(1-(4-chlorophenyl)-1,2,3,4-tetrahydroquinolin-3-yl)acrylamide (Cpd. No. 033)

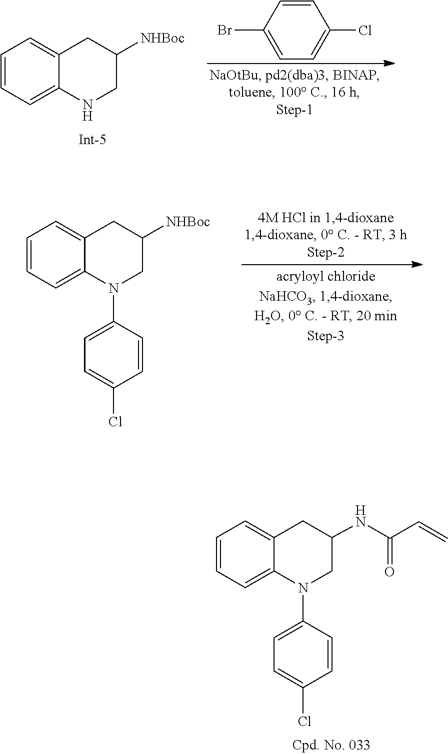 [SMILES: CC(C)(C)OC(=O)NC1CNc2ccccc2C1 ; Clc1ccc(Br)cc1 ; CC(C)(C)OC(=O)NC1Cc2ccccc2N(c2ccc(Cl)cc2)C1 ; C=CC(=O)NC1Cc2ccccc2N(c2ccc(Cl)cc2)C1]

Step 1: A solution of tert-butyl (1,2,3,4-tetrahydroquinolin-3-yl)carbamate (Int-5) (500 mg, 2.01 mmol) in toluene (20 mL) (sealed tube) was treated with 1-bromo-4-chlorobenzene (1.35 g, 7.05 mmol), NaOtBu (359 mg, 3.74 mmol) and degassed with argon for 5 min. Pd₂(dba)₃ (73 mg, 0.08 mmol), BINAP (100 mg, 0.161 mmol) were added to the reaction mixture and stirred at 100° C. for 16 h. The reaction progress was monitored by TLC. TLC mobile phase: 20% EtOAc in pet ether, RF: 0.70, TLC detection: UV. The reaction mixture was concentrated under reduced pressure to afford crude product as a brown gum (2.0 g, LC/MS 19%) which was purified by column chromatography using silica 100-200 mesh and 10% of EtOAc in pet ether as an eluent to afford tert-butyl (1-(4-chlorophenyl)-1,2,3,4-tetrahydroquinolin-3-yl)carbamate as an off-white solid (500 mg, 62%). (LC/MS; m/z 359.2 [M+H]⁺)

Steps 2-3: These steps were executed in a manner similar (use of appropriate reagents and purification methods known to the person skilled in the art) to Cpd. No. 001. Starting material (150 mg, 0.51 mmol, LC/MS 76%) yielded a pale yellow solid which was purified by preparative HPLC method H4. The collected fractions were concentrated under reduced pressure and lyophilised to afford N-(1-(4-chlorophenyl)-1,2,3,4-tetrahydroquinolin-3-yl)acrylamide (Cpd. No. 033) as an off-white solid (33 mg, 27%). (LC/MS; m/z 313.2 [M+H]⁺) The following compounds were prepared in a manner similar to Cpd. No. 033 by using appropriate reagents and purification methods known to the person skilled in the art: Cpd. No. 034, Cpd. No. 035, and Cpd. No. 036.

### Examples 8-9: Synthesis of N-(1-(4-(trifluoromethyl)phenyl)-1,2,3,4-tetrahydroquinolin-3-yl)propionamide (Cpd. No. 037) and 3-methyl-N-(1-(4-(trifluoromethyl)phenyl)-1,2,3,4-tetrahydroquinolin-3-yl)but-2-enamide (Cpd. No. 038)

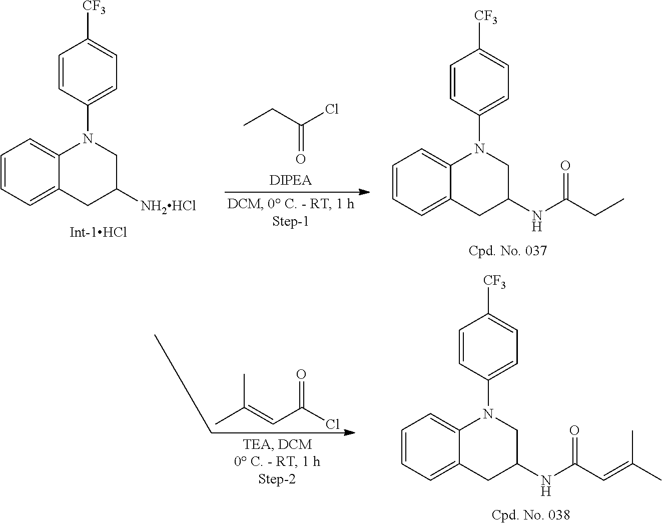

Step 1: A solution of 1-(4-(trifluoromethyl)phenyl)-1,2,3,4-tetrahydroquinolin-3-amine hydrochloride (Int-1·HCl) (35 mg, 0.1 mmol) in DCM (3 mL) was cooled to 0° C., treated with TEA (0.03 mL, 0.16 mmol) and propionyl chloride (7.8 mg, 0.08 mmol) under a nitrogen atmosphere. The reaction mixture was stirred for 1 h at RT. Progress of the reaction was monitored by TLC. TLC mobile phase: 50% EtOAc in pet ether, RF: 0.40, TLC detection: UV. The reaction mixture was diluted with cold water (10 mL) and the product was extracted with DCM (10 mL). The organic layer was separated, dried over Na₂SO₄ and evaporated under reduced pressure to afford crude product (40 mg). Additional 45 mg of crude product from another batch was mixed and purified by preparative HPLC method H5. The collected fractions were evaporated under lyophilisation to afford N-(1-(4-(trifluoromethyl)phenyl)-1,2,3,4-tetrahydroquinolin-3-yl)propionamide (Cpd. No. 037) as an off-white solid (17 mg, 23%) (LC/MS; m/z 349.2 [M+H]⁺)

Step 2: A solution of 1-(4-(trifluoromethyl)phenyl)-1,2,3,4-tetrahydroquinolin-3-amine hydrochloride (Int-1·HCl) (150 mg, 0.45 mmol) in DCM (5 mL) was treated with TEA (69.2 mg, 0.68 mmol) and 3-methylbut-2-enoyl chloride (65 mg, 0.54 mmol) at 0° C. The reaction mixture was stirred at RT for 1 h. Progress of the reaction was monitored by TLC. TLC mobile phase: 50% EtOAc in pet ether, RF: 0.5, TLC detection: UV. The reaction mixture was diluted with cold water (20 mL) and extracted with EtOAc (2×10 mL). The organic layer was separated, dried over Na₂SO₄, filtered and the filtrate was evaporated under reduced pressure to afford crude product as a yellow liquid (160 mg, LC/MS 67%) which was purified by preparative HPLC method H4. The collected fractions were lyophilised to afford 3-methyl-N-(1-(4-(trifluoromethyl)phenyl)-1,2,3,4-tetrahydroquinolin-3-yl)but-2-enamide (Cpd. No. 038) as an off-white solid (16 mg, 9%) (LC/MS; m/z 375.2 [M+H]⁺)

### Example 10: Synthesis of 2-fluoro-N-(1-(4-(trifluoromethyl)phenyl)-1,2,3,4-tetrahydroquinolin-3-yl)acrylamide (Cpd. No. 039)

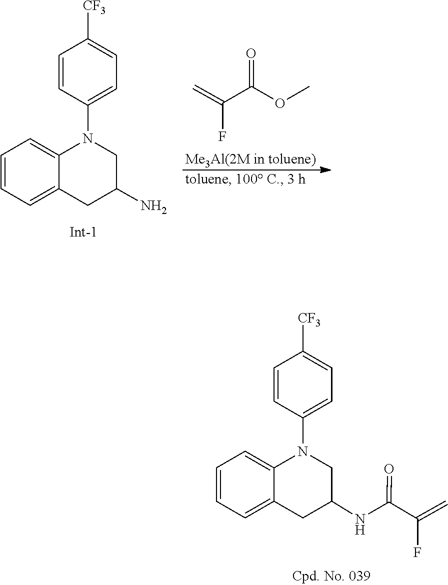

A solution of 1-(4-(trifluoromethyl)phenyl)-1,2,3,4-tetrahydroquinolin-3-amine (Int-1) (200 mg, 0.68 mmol) in toluene (5.0 mL) was treated with methyl 2-fluoroacrylate (108 mg, 1.03 mmol) and Me₃Al (2M in toluene, 1.02 mL, 2.05 mmol) at RT (sealed tube). The reaction mixture was stirred at 100° C. for 3 h. Progress of the reaction was monitored by TLC. TLC mobile phase: 10% MeOH in DCM, RF: 0.51, TLC detection: UV. The reaction mixture was cooled to RT, diluted with EtOAc (10 mL) and water (10 mL). The organic layer was separated, washed with brine (10 mL), dried over Na₂SO₄ and concentrated under reduced pressure to afford crude product (250 mg, LC/MS 46%) which was purified by normal phase column chromatography using a 24 g column and a gradient of 40% EtOAc in pet ether as an eluent to afford 2-fluoro-N-(1-(4-(trifluoromethyl)phenyl)-1,2,3,4-tetrahydroquinolin-3-yl)acrylamide (Cpd. No. 039) as a pale yellow solid (109 mg, 43%). (LC/MS; m/z 365.2 [M+H]⁺)

### Example 11: Synthesis of N-(1-(4-(trifluoromethyl)phenyl)-1,2,3,4-tetrahydroquinolin-3-yl)propiolamide (Cpd. No. 040)

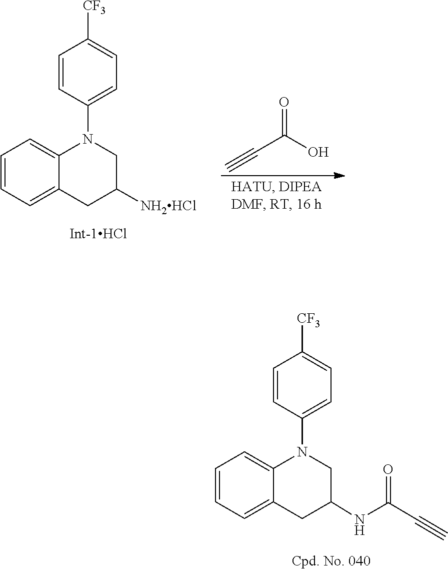

A solution of propiolic acid (32.01 mg, 0.457 mmol) and 1-(4-(trifluoromethyl)phenyl)-1,2,3,4-tetrahydroquinolin-3-amine hydrochloride (Int-1·HCl) (150 mg, 0.457 mmol, LC/MS 96%) in DMF (4 mL) was treated with HATU (260 mg, 0.683 mmol) and DIPEA (147.3 mg, 1.141 mmol) at 0° C. under a nitrogen atmosphere. The reaction mixture was stirred at RT for 16 h. The reaction progress was monitored by TLC. TLC mobile phase: 50% EtOAc in pet ether, RF: 0.6, TLC detection: UV. The reaction mixture was diluted with EtOAc (20 mL), washed with ice water (2×20 mL) and brine (10 mL). The organic layer was dried over Na₂SO₄ and concentrated to afford crude product as a pale brown gum (115 mg, LC/MS 77%) which was purified preparative HPLC method H3. The collected fractions were lyophilised to afford N-(1-(4-(trifluoromethyl)phenyl)-1,2,3,4-tetrahydroquinolin-3-yl)propiolamide as an off-white solid (Cpd. No. 040) (33 mg, 21%) (LC/MS; 345.2m/z [M+H]⁺)

The following compounds were prepared in a manner similar to Cpd. No. 040 by using appropriate reagents and purification methods known to the person skilled in the art: Cpd. No. 041 and Cpd. No. 042. For compound Cpd. No. 042 2-cyanoacrylic acid was used. The reaction product cyclized upon purification.

### Examples 12-13: Synthesis of 2-((1-(4-(trifluoromethyl)phenyl)-1,2,3,4-tetrahydroquinolin-3-yl)amino)ethan-1-ol (Cpd. No. 043) and (1-(4-(trifluoromethyl)phenyl)-1,2,3,4-tetrahydroquinolin-3-yl)glycine (Cpd. No. 044)

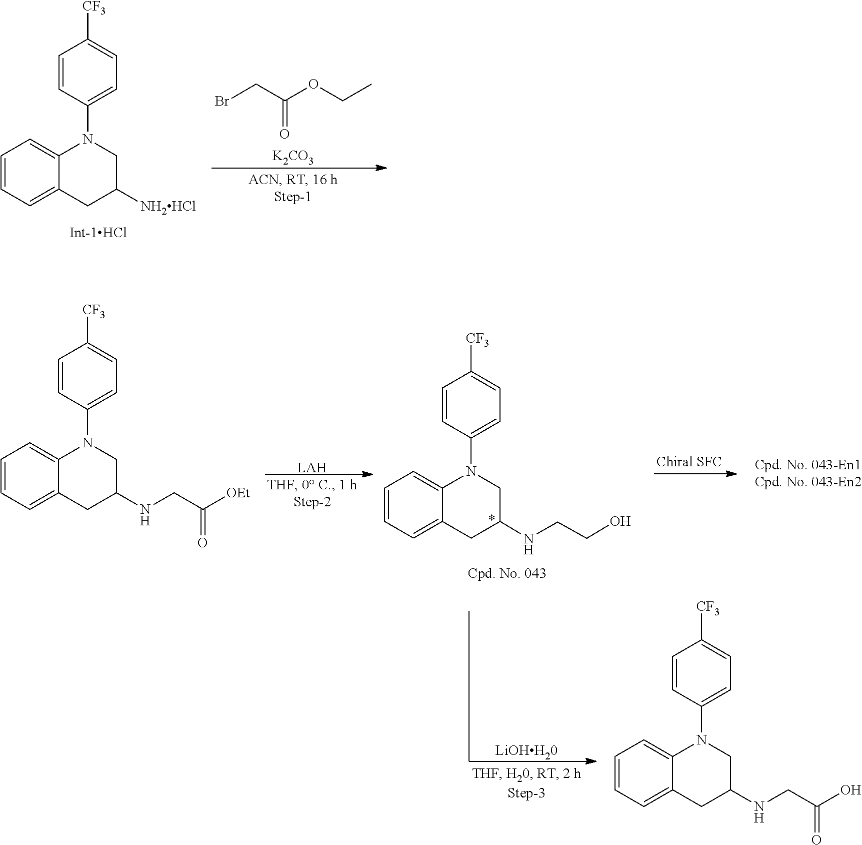

Step 1: A solution of 1-(4-(trifluoromethyl)phenyl)-1,2,3,4-tetrahydroquinolin-3-amine hydrochloride (Int-1·HCl) (100 mg, 0.3 mmol) in ACN (4 mL) was treated with K₂CO₃ (105 mg, 0.76 mmol) and ethyl 2-bromoacetate (50 mg, 0.30 mmol) at RT and stirred for 16 h at RT under a nitrogen atmosphere. Progress of the reaction was monitored by TLC. TLC mobile phase: 50% EtOAc in pet ether, RF: 0.5, TLC detection: UV. The reaction mixture was diluted with cold water (20 mL) and extracted with EtOAc (20 mL). The organic layer was dried over Na₂SO₄ and evaporated under reduced pressure to afford ethyl (1-(4-(trifluoromethyl)phenyl)-1,2,3,4-tetrahydroquinolin-3-yl)glycinate (130 mg, 90%) as a yellow liquid which was taken forward in the subsequent reaction without further purification. (LC/MS; m/z 379.2 [M+H]⁺)

Step 2: A solution of ethyl (1-(4-(trifluoromethyl)phenyl)-1,2,3,4-tetrahydroquinolin-3-yl)glycinate (130 mg, 0.34 mmol, LC/MS 80%) in THF (2 mL) was cooled to 0° C. and treated with LAH (2M in THF, 0.17 mL, 0.34 mmol) under a nitrogen atmosphere. The reaction mixture was stirred at RT for 1 h. Progress of the reaction was monitored by TLC. TLC mobile phase: 50% EtOAc in pet ether, RF: 0.3, TLC detection: UV. The reaction mixture was quenched with aq. NH₄Cl solution (10 mL) at 0° C. and extracted with EtOAc (10 mL). The organic layer was separated, dried over Na₂SO₄ and evaporated under reduced pressure to afford crude product (100 mg. LC/MS 85%), which was purified preparative HPLC method H4. The collected fractions were lyophilised to afford 2-((1-(4-(trifluoromethyl)phenyl)-1,2,3,4-tetrahydroquinolin-3-yl)amino)ethan-1-ol (Cpd. No. 043) as an off-white solid (25 mg, 27%). (LC/MS; m/z 337.2 [M+H]⁺). Chiral SFC purification: 60 mg of Cpd. No. 043 was further purified by preparative SFC method K2 to afford Cpd. No. 043-En1 (7 mg) and Cpd. No. 043-En2 (9 mg), both as an off-white gum. (LC/MS; m/z 337.2 [M+H]⁺). The chiral purity of both enantiomers was assessed by analytic SFC method S2: Cpd. No. 043-En1, 96.1% ee; Cpd. No. 043-En2, 95.3% ee.

Step 3: A stirred solution of ethyl (1-(4-(trifluoromethyl)phenyl)-1,2,3,4-tetrahydroquinolin-3-yl)glycinate (120 mg, 0.31 mmol, LC/MS 71%) in THF (3 mL) and water (1 mL) was treated with LiOH·H₂O (33.3 mg, 0.7 mmol) at 0° C. The reaction mixture was stirred for 2 h at RT. Progress of the reaction was monitored by TLC. TLC mobile phase: 50% EtOAc in pet ether, TLC detection: UV. The reaction mixture was acidified with 2M HCl solution (pH 5) and extracted with EtOAc (2×10 mL). The organic layer was dried over Na₂SO₄ and evaporated under reduced pressure to afford crude product (55 mg, LC/MS 90%), which was purified by preparative HPLC method H10. The collected fractions were lyophilised to afford (1-(4-(trifluoromethyl)phenyl)-1,2,3,4-tetrahydroquinolin-3-yl)glycine (Cpd. No. 044) as an off-white solid (24 mg, 30%) (LC/MS; m/z 351.2 [M+H]⁺)

The following compounds were prepared in a manner similar to Cpd. No. 043 and Cpd. No. 044, respectively by using appropriate reagents and purification methods known to the person skilled in the art: Cpd. No. 045 and Cpd. No. 046. The enantiomers of Cpd. No. 045 were by preparative SFC method K3. The chiral purity of both enantiomers was assessed by analytic SFC method S2: Cpd. No. 045-En1, 94.9% ee; Cpd. No. 045-En2, 91.2% ee.

### Examples 14-16: Synthesis of N-(1-(4-(trifluoromethyl)phenyl)-1,2,3,4-tetrahydroquinolin-3-yl)methanesulfonamide (Cpd. No. 047), N-(1-(4-(trifluoromethyl)phenyl)-1,2,3,4-tetrahydroquinolin-3-yl)ethenesulfonamide (Cpd. No. 048), and N-(2-(methylsulfonyl)ethyl)-1-(4-(trifluoromethyl)phenyl)-1,2,3,4-tetrahydroquinolin-3-amine (Cpd. No. 049)

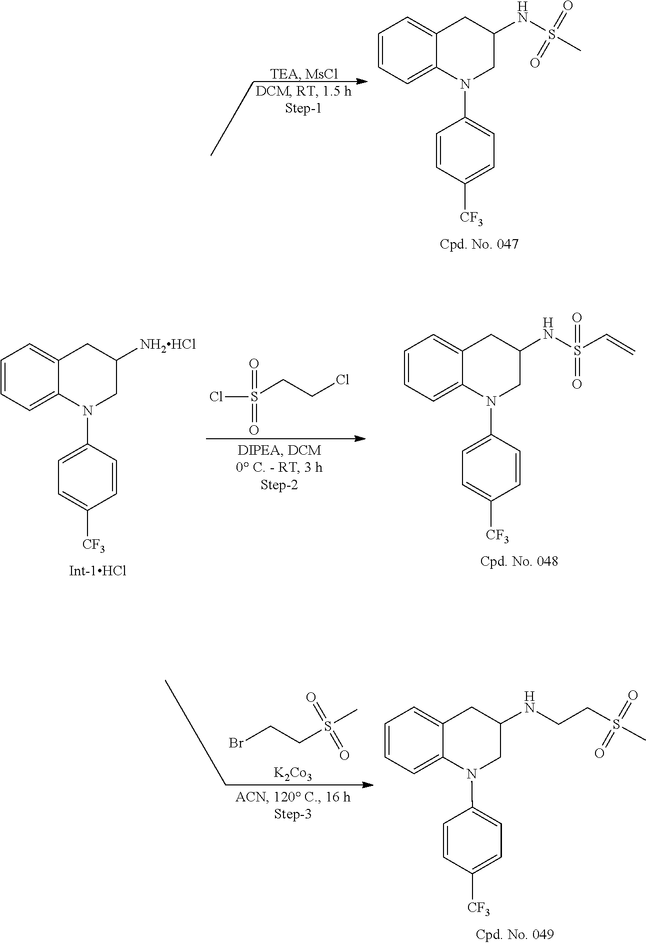

Step 1: A solution of 1-(4-(trifluoromethyl)phenyl)-1,2,3,4-tetrahydroquinolin-3-amine hydrochloride (150 mg, 0.45 mmol) (Int-1·HCl) in DCM (5 mL) was treated with TEA (115 mg, 1.1 mmol) and methanesulfonyl chloride (62.5 mg, 0.54 mmol) at 0° C. The reaction mixture was stirred at RT for 1.5 h under a nitrogen atmosphere and monitored by TLC. TLC mobile phase: 50% EtOAc in pet ether, RF: 0.5, TLC detection: UV. The reaction mixture was diluted with cold water (20 mL) and extracted with EtOAc (2×10 mL). The organic layer was separated, dried over Na₂SO₄, filtered and the filtrate was evaporated under reduced pressure to afford crude product as a yellow liquid (120 mg, LC/MS 75%), which was purified by preparative HPLC method H5. The collected fractions were lyophilised to afford N-(1-(4-(trifluoromethyl)phenyl)-1.2.3.4-tetrahydroquinolin-3-yl)methane sulfonamide (Cpd. No. 047) as an off-white solid (14 mg, 9%). (LC/MS; m/z 371.2 [M+H]⁺)

Step 2: A solution of 1-(4-(trifluoromethyl)phenyl)-1,2,3,4-tetrahydroquinolin-3-amine hydrochloride (200 mg, 0.60 mmol) (Int-1·HCl) in DCM (10 mL) was cooled to 0° C., treated with DIPEA (157 mg, 1.21 mmol) and 2-chloroethane-1-sulfonyl chloride (99 mg, 0.60 mmol) under a nitrogen atmosphere. The reaction mixture was stirred for 3 h at RT. The reaction progress was monitored by TLC. TLC mobile phase: 30% EtOAc in pet ether, RF: 0.6, TLC detection: UV. The reaction mixture was diluted with water (10 mL) and extracted with DCM (20 mL). The organic layer was separated, dried over Na₂SO₄, filtered and concentrated under reduced pressure to afford crude product (260 mg) as a pale brown liquid which was purified by normal phase chromatography (Grace) using a 12 g reveleris column and a gradient of 20% EtOAc in pet ether as an eluent to afford the product (120 mg, LC-MS 87%) as an off-white gum. The product was further purified by preparative HPLC method H5. The collected fractions were concentrated under reduced pressure and lyophilised to afford N-(1-(4-(trifluoromethyl)phenyl)-1,2,3,4-tetrahydroquinolin-3-yl)ethenesulfonamide (Cpd. No. 048) as an off-white solid (42 mg, 18%). (LC/MS; m/z 383.2 [M+H]⁺)

Step 3: A solution of 1-(4-(trifluoromethyl)phenyl)-1,2,3,4-tetrahydroquinolin-3-amine hydrochloride (Int-1·HCl) (120 mg, 0.36 mmol) in ACN (3 mL) (sealed tube) was treated with K₂CO₃ (149 mg, 1.08 mmol), 1-bromo-2-(methylsulfonyl)ethane (235 mg, 1.25 mmol) at RT and stirred at 120° C. for 16 h. Progress of the reaction was monitored by TLC. TLC mobile phase: 10% MeOH in DCM, RF: 0.47, TLC detection: UV. The reaction mixture was cooled at RT, diluted with water (30 mL) and the product was extracted with DCM (2×30 mL). The organic layer was dried over Na₂SO₄ and evaporated under reduced pressure to afford crude product as a pale yellow gum (120 mg, LC/MS 55.94%). To this batch was added 25 mg (LC/MS 54%) from a second batch and the combined batches were preparative HPLC method H7. The collected fractions were concentrated under reduced pressure and lyophilised to afford N-(2-(methylsulfonyl)ethyl)-1-(4-(trifluoromethyl)phenyl)-1,2,3,4-tetrahydroquinolin-3-amine (Cpd. No. 049) as a pale yellow gum (13 mg, 9%). (LC/MS; m/z 399.2 [M+H]⁺)

### Examples 17-18: Synthesis of 2-(1-(4-(trifluoromethyl)phenyl)-1,2,3,4-tetrahydroquinolin-3-yl)-1,2-thiazetidine-1,1-dioxide (Cpd. No. 050) and N-methyl-2-((1-(4-(trifluoromethyl)phenyl)-1,2,3,4-tetrahydroquinolin-3-yl)amino)ethane-1-sulfonamide (Cpd. No. 051)

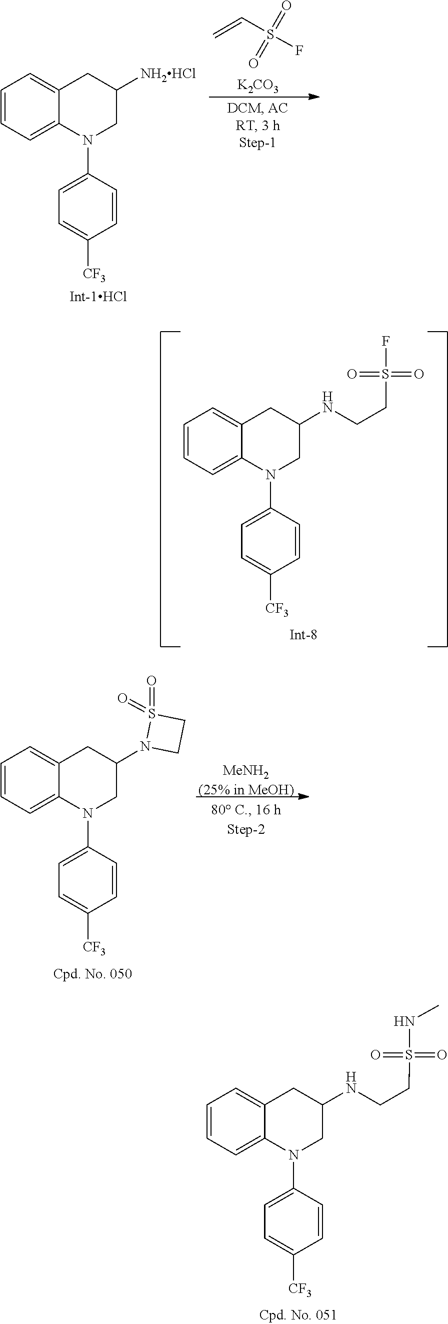

Step 1: A solution of 1-(4-(trifluoromethyl)phenyl)-1,2,3,4-tetrahydroquinolin-3-amine hydrochloride (Int-1·HCl) (300 mg, 0.91 mmol) in DCM (3 mL) and ACN (3 mL) was treated with K₂CO₃ (252 mg, 1.82 mmol) and ethanesulfonyl fluoride (101 mg, 0.91 mmol) at 0° C. under a nitrogen atmosphere. The reaction mixture was stirred at RT for 3 h. The reaction progress was monitored by TLC. TLC mobile phase: 30% EtOAc in pet ether, RF: 0.46, TLC detection: UV. The reaction mixture was diluted with DCM (10 mL) and water (10 mL). The organic layer was separated, dried over Na₂SO₄, filtered and concentrated under reduced pressure to afford crude product as an off-white gum (350 mg, LC/MS 78% Int-8; m/z 403.1 [M+H]⁺). The crude product was purified by normal phase chromatography (Grace) using a 12 g reveleris column (silica gel) and a gradient of 20% EtOAc in pet ether as an eluent to afford 2-(1-(4-(trifluoromethyl)phenyl)-1,2,3,4-tetrahydroquinolin-3-yl)-1,2-thiazetidine-1,1-dioxide (Cpd. No. 051) as an off-white solid (170 mg, 52%). (LC/MS; m/z 383.2 [M+H]⁺). During column purification Int-8 is cyclized into Cpd. No. 050.

Step 2: A solution of 2-((1-(4-(trifluoromethyl)phenyl)-1,2,3,4-tetrahydroquinolin-3-yl)amino)ethane-1-sulfonylfluoride (Int-8) (100 mg, LC/MS 67%) in MeOH (2 mL) (sealed tube) was treated with methyl amine (25% in MeOH, 2 mL) and stirred for 2 h at 80° C. The reaction progress was monitored by TLC. TLC mobile phase: 5% MeOH in DCM, RF: 0.43, TLC detection: UV. The reaction mixture was concentrated under reduced pressure to afford crude product (90 mg, LC/MS 84%) which was purified by preparative HPLC method H7. The collected fractions were concentrated under reduced pressure and lyophilised to afford N-methyl-2-((1-(4-(trifluoromethyl)phenyl)-1,2,3,4-tetrahydroquinolin-3-yl)amino)ethane-1-sulfonamide as a pale yellow gum (Cpd. No. 051) (10 mg, 14%). (LC/MS; m/z 414.3 [M+H]⁺)

### Examples 19-21: Synthesis of 2-cyano-N-(1-(4-(trifluoromethyl)phenyl)-1,2,3,4-tetrahydroquinolin-3-yl)acetamide (Cpd. No. 052), 3-((1-(4-(trifluoromethyl)phenyl)-1,2,3,4-tetrahydroquinolin-3-yl)amino)propanenitrile (Cpd. No. 053), and 2-((1-(4-(trifluoromethyl)phenyl)-1,2,3,4-tetrahydroquinolin-3-yl)amino)acetonitrile (Cpd. No. 054)

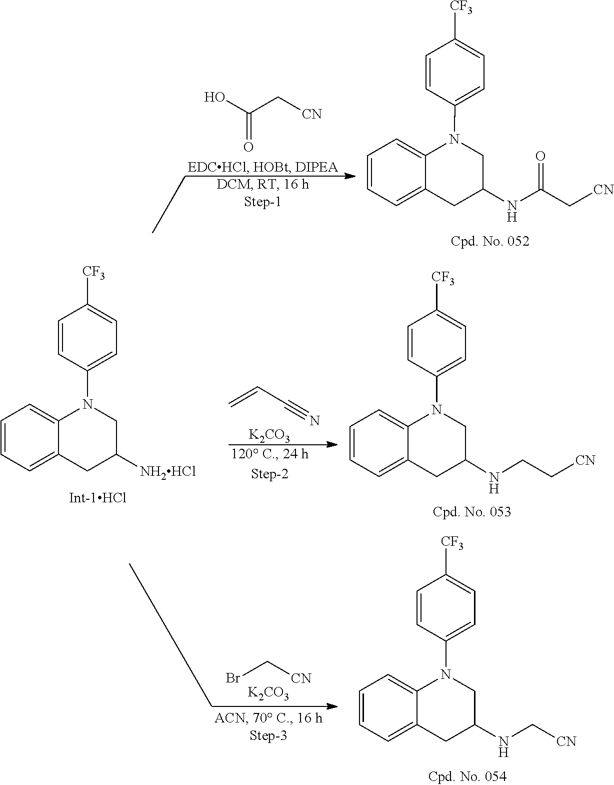

Step 1: A solution of 2-cyanoacetic acid (97 mg, 1.14 mmol) in DCM (4 mL) was treated with DIPEA (176 mg, 1.37 mmol), EDC·HCl (219 mg, 1.14 mmol) and HOBt (154 mg, 1.14 mmol) at RT under a nitrogen atmosphere and stirred for 10 min. 1-(4-(trifluoromethyl)phenyl)-1,2,3,4-tetrahydroquinolin-3-amine hydrochloride (Int-1·HCl) (150 mg, 0.45 mmol, LC/MS 83%) was added to the reaction mixture and stirred at RT for 8 h. Progress of the reaction was monitored TLC. TLC mobile phase: 5% MeOH in DCM, RF: 0.47, TLC detection: UV. The reaction mixture was diluted with water (20 mL) and extracted with DCM (2×25 mL). The combined organic layer was dried over Na₂SO₄, filtered and concentrated under reduced pressure to afford crude product as a yellow gum (320 mg, LC/MS 60%) which was purified by preparative HPLC method H5. The collected fractions were concentrated under reduced pressure and lyophilised to afford 2-cyano-N-(1-(4-(trifluoromethyl)phenyl)-1,2,3,4-tetrahydroquinolin-3-yl)acetamide (Cpd. No. 052) as an off-white solid (75 mg, 55%). (LC/MS; m/z 360.2 [M+H]⁺)

Step 2: A solution of 1-(4-(trifluoromethyl)phenyl)-1,2,3,4-tetrahydroquinolin-3-amine hydrochloride (Int-1·HCl) (100 mg, 0.30 mmol, LC/MS 92%) in acrylonitrile (10 mL) (sealed tube) was treated with K₂CO₃ (126 mg, 0.91 mmol) at RT. The reaction mixture was stirred at 120° C. for 24 h. Progress of the reaction was monitored by TLC. TLC mobile phase: 5% MeOH in DCM, RF: 0.73, TLC detection: UV. The reaction mixture was cooled to RT, diluted with water (50 mL) and extracted with EtOAc (2×50 mL). The combined organic layer was dried over Na₂SO₄, filtered and concentrated under reduced pressure to afford crude product as a brown gum (200 mg, LC/MS 58%), which was purified by preparative HPLC method H3. The collected fractions were concentrated under reduced pressure and lyophilised to afford 3-((1-(4-(trifluoromethyl)phenyl)-1,2,3,4-tetrahydroquinolin-3-yl)amino)propanenitrile (Cpd. No. 053) as a pale yellow gum (34 mg, 36%). (LC/MS; m/z 346.2 [M+H]⁺)

Step 3: A solution of 1-(4-(trifluoromethyl)phenyl)-1,2,3,4-tetrahydroquinolin-3-amine hydrochloride (Int-1·HCl) (150 mg, 0.45 mmol, LC/MS 83%) in ACN (3 mL) (sealed tube) was treated with K₂CO₃ (189 mg, 1.37 mmol) and 2-bromoacetonitrile (65 mg, 0.54 mmol) at RT. The reaction mixture was stirred at 70° C. for 16 h. Progress of the reaction was monitored by TLC. TLC mobile phase: 5% MeOH in DCM, RF: 0.67, TLC detection: UV. The reaction mixture was cooled to RT, diluted with water (20 mL) and extracted with EtOAc (2×25 mL). The combined organic layer was dried over Na₂SO₄, filtered and concentrated under reduced pressure to afford crude product as a brown gum (210 mg, LC/MS 64%) which was purified by preparative HPLC method H43. The collected fractions were concentrated under reduced pressure and lyophilised to afford 2-((1-(4-(trifluoromethyl)phenyl)-1,2,3,4-tetrahydroquinolin-3-yl)amino)acetonitrile (Cpd. No. 054) as an off-white solid (43 mg, 34%). (LC/MS; m/z 332.2 [M+H]⁺)

### Example 22: Synthesis of tert-butyl (5-bromo-1-(4-(trifluoromethyl)phenyl)-1,2,3,4-tetrahydroquinolin-3-yl)carbamate (Cpd. No. 055)

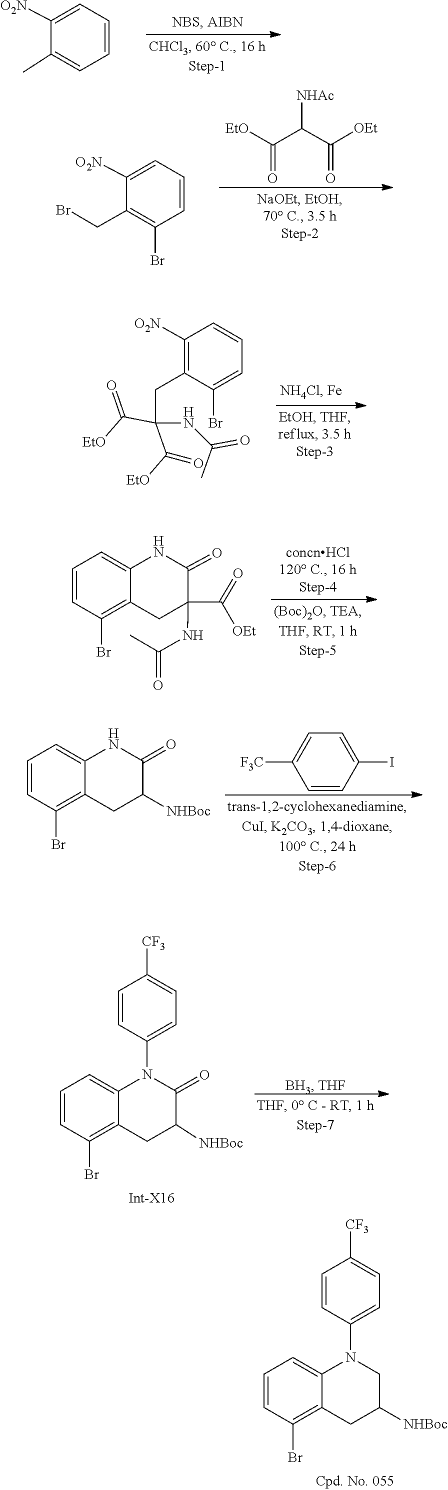

Step 1: A solution of 1-bromo-2-methyl-3-nitrobenzene (30.0 g, 138.9 mmol) in CHCl₃ (300 mL) was treated with AIBN (2.2 g, 13.8 mmol) and NBS (51.9 g, 291.6 mmol) at RT. The reaction mixture was stirred for 16 h at 60° C. Progress of the reaction was monitored by TLC. TLC mobile phase: 5% EtOAc in pet ether, RF: 0.3, TLC detection: UV. The reaction mixture was cooled to RT and diluted with ice water (400 mL). The organic layer was separated and the aqueous layer was extracted with DCM (3×40 mL). The combined organic layer was dried over Na₂SO₄ and evaporated under reduced pressure to afford crude product (40 g) which was purified by flash chromatography using a 120 g reveleris column and eluted with 10% EtOAc in pet ether gradient to afford 1-bromo-2-(bromomethyl)-3-nitrobenzene as an off-white solid (36 g, 87%).

Step 2: Diethyl 2-acetamidomalonate (30.0 g, 138.1 mmol) was treated with a solution of freshly prepared NaOEt solution (3.3 g of Na metal was dissolved in in 150 mL EtOH at 0° C. under a nitrogen atmosphere) at RT and stirred at 50° C. for 1 h. The resulting reaction mixture was treated with 1-bromo-2-(bromomethyl)-3-nitrobenzene (40.4 g, 138.1 mmol) and KI (1.1 g, 6.9 mmol) at 50° C. and stirred at 70° C. for 2.5 h. Progress of the reaction was monitored by TLC. TLC mobile phase: 30% EtOAc in pet ether, RF: 0.2, TLC detection: UV. The reaction mixture was diluted with ice water (250 mL) and the product was extracted with EtOAc (3×50 mL). The organic layer was dried over Na₂SO₄ and evaporated under reduced pressure to afford crude product (37.0 g) which was purified by flash chromatography (Grace) using a 120 g reveleris column and a gradient of 50% EtOAc in pet ether to afford diethyl 2-acetamido-2-(2-bromo-6-nitrobenzyl)malonate as an off-white solid (25 g, 42%). (LC/MS; m/z 430.9 [M+H]⁺)

Step 3: A sat. NH₄Cl solution (48 mL) and iron powder (11.9 g, 214 mmol) were added to a solution of diethyl 2-acetamido-2-(2-bromo-6-nitrobenzyl)malonate (23.0 g, 53.5 mmol) in EtOH (160 mL) and THF (70 mL) at RT. The reaction mixture was heated to reflux for 3.5 h. Progress of the reaction was monitored by TLC. TLC mobile phase: 50% EtOAc in pet ether, RF: 0.5, TLC detection: UV. The reaction mixture was filtered through a celite pad and washed several times with MeOH (90 mL). The filtrate was evaporated, diluted with EtOAc (250 mL) and washed with water (3×50 mL). The organic layer was dried over Na₂SO₄ and evaporated under reduced pressure to afford ethyl 3-acetamido-5-bromo-2-oxo-1,2,3,4-tetrahydroquinoline-3-carboxylate as an off-white solid (18.5 g, 53%). (LC/MS; m/z 355.1 [M+H]⁺)

Step 4: Ethyl 3-acetamido-5-bromo-2-oxo-1,2,3,4-tetrahydroquinoline-3-carboxylate (18.5 g, 52.3 mmol, LC/MS 55%) was dissolved in conc. HCl (190 mL) and heated to reflux for 16 h. Progress of the reaction was monitored by TLC. TLC mobile phase: 50% EtOAc in pet ether. The reaction mixture was cooled to RT, solid material was filtered, washed with cold water (80 mL) and dried under reduced pressure to afford 3-amino-5-bromo-3,4-dihydroquinolin-2(1H)-one hydrochloride as an off-white solid (8.3 g, 100%). (LC/MS; m/z 241.3 [M+H]⁺). The product was used without further purification in the next step.

Step 5: A solution of 3-amino-5-bromo-3,4-dihydroquinolin-2(1H)-one hydrochloride (8.0 g, 29 mmol, LC/MS 88%) in THF (75 mL) was cooled to 0° C., treated with TEA (16.3 mL, 123 mmol) followed by a solution of (Boc)₂O (6.0 mL, 27.7 mmol) in THF (5 mL) under an argon atmosphere. The solution was stirred at RT for 1 h. The reaction was monitored by TLC. TLC mobile phase: 20% EtOAc in pet ether, RF: 0.53, TLC detection: UV. The reaction mixture was diluted with cold water (140 mL) and extracted with EtOAc (3×25 mL). The organic layer was separated, dried over Na₂SO₄ and evaporated under reduced pressure to afford crude product (9.0 g) which was purified by flash chromatography (Grace) using a 80 g reveleris column (silica gel) and 20% EtOAc in pet ether gradient to afford tert-butyl (5-bromo-2-oxo-1,2,3,4-tetrahydroquinolin-3-yl)carbamate as an off-white solid (7.7 g, 70%). (LC/MS; m/z 340.8 [M+H]⁺)

Step 6: A solution of tert-butyl (5-bromo-2-oxo-1,2,3,4-tetrahydroquinolin-3-yl)carbamate (8.0 g, 23.5 mmol, LC/MS 91%) in 1,4-dioxane (80 mL) was treated with 1-iodo-4-(trifluoromethyl)benzene (12.7 g, 47 mmol), trans-1,2-cyclohexanediamine (535 mg, 4.7 mmol), Cul (894 mg, 4.7 mmol) and K₂CO₃ (8.1 g, 58.7 mmol) under argon at RT. The reaction mixture was stirred at 100° C. for 24 h. Progress of the reaction was monitored by TLC. TLC mobile phase: 20% EtOAc in pet ether. The reaction mixture was filtered through a celite pad and the pad was washed with EtOAc (50 mL). The filtrate was evaporated under reduced pressure to afford crude product (9.0 g) which was purified by flash chromatography (Grace) using a 80 g reveleris column and 20% EtOAc in pet ether gradient to afford tert-butyl (5-bromo-2-oxo-1-(4-(trifluoromethyl)phenyl)-1,2,3,4-tetrahydroquinolin-3-yl)carbamate (Int-X15) as an off-white solid (1.2 g, 9%). (LC/MS; m/z 485.1 [M+H]⁺)

Step 7: A solution of Int-X15 (600 mg, 1.2 mmol, LC/MS 80%) in THF (4 mL) was cooled to 0° C. and treated with BH₃.THF (1M in THF, 6.0 mL, 6.1 mmol) under a nitrogen atmosphere. The reaction mixture was stirred at RT for 1 h. Progress of the reaction was monitored by TLC. TLC mobile phase: 20% EtOAc in pet ether, RF: 0.5, TLC detection: UV. The reaction mixture was cooled to 0° C., quenched with MeOH (4 mL) and evaporated under reduced pressure to afford crude product (700 mg) which was purified by flash chromatography (Grace) using a 40 g reveleris column and eluted with 40% EtOAc in pet ether gradient to afford tert-butyl (5-bromo-1-(4-(trifluoromethyl)phenyl)-1,2,3,4-tetrahydroquinolin-3-yl)carbamate (320 mg, 51%, LC/MS 76%). 100 mg was further purified preparative HPLC method H3 The collected fractions were evaporated and lyophilised to afford tert-butyl (5-bromo-1-(4-(trifluoromethyl)phenyl)-1,2,3,4-tetrahydroquinolin-3-yl)carbamate (Cpd. No. 055) (42 mg, 56%) as an off-white solid. (LC/MS; m/z 471.2 [M+H]⁺)

The intermediate Int-X16 was prepared in a manner similar (use of appropriate reagents and purification methods known to the person skilled in the art) to Int-X15:

| Cpd. | | [M + H]⁺ |
|---|---|---|
| Nr. | Structure | (m/z) |
| Int-X16 | 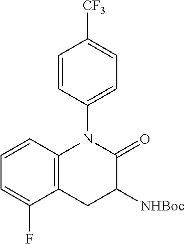 [SMILES: CC(C)(C)OC(=O)NC1Cc2c(F)cccc2N(c2ccc(C(F)(F)F)cc2)C1=O] | 425.3 |

Compound Cpd. No. 056 (employing 1-fluoro-2-methyl-3-nitrobenzene at step 1) was prepared in a manner similar to Cpd. No. 055 by using appropriate reagents and purification methods known to the person skilled in the art.

### Example 23: Synthesis of tert-butyl (6-bromo-1-(4-(trifluoromethyl)phenyl)-1,2,3,4-tetrahydroquinolin-3-yl)carbamate (Cpd. No. 057)

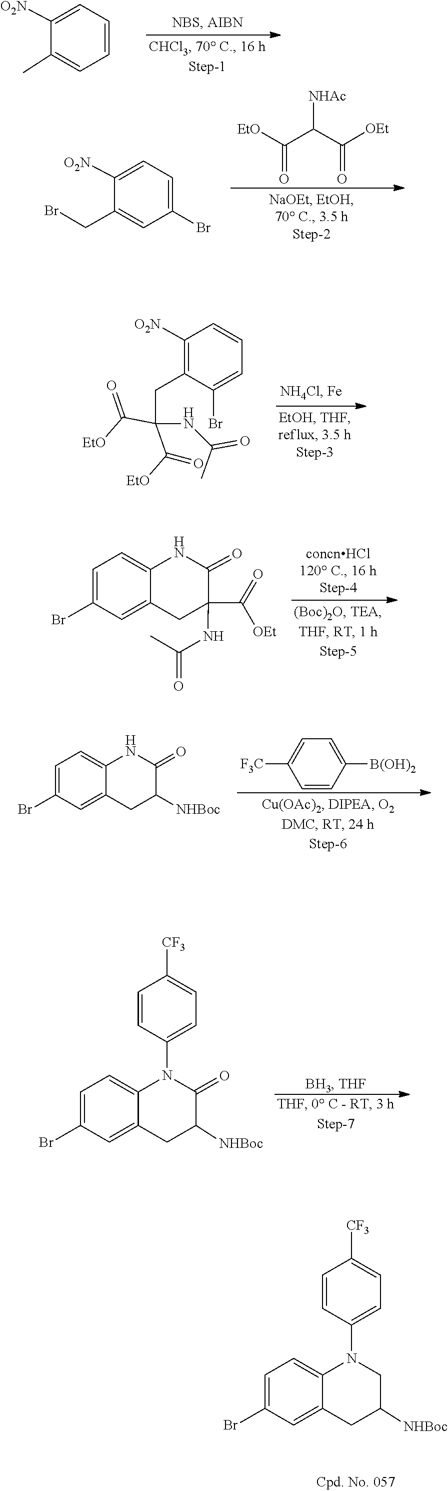

Step 1: A solution of 4-bromo-2-methyl-3-nitrobenzene (20.0 g, 92.6 mmol) in CHCl₃ (200 mL) was treated with AIBN (1.51 g, 9.3 mmol) and NBS (32.96 g, 185.2 mmol) at RT. The reaction mixture was stirred at 70° C. for 16 h. Progress of the reaction was monitored by TLC. TLC mobile phase: 7% EtOAc in pet ether, RF: 0.2, TLC detection: UV. The reaction mixture was cooled to RT and diluted with ice water (200 mL). The organic layer was separated and the aqueous layer was extracted with DCM (3×150 mL). The combined organic layer was dried over Na₂SO₄ and evaporated under reduced pressure to afford crude product (22 g) which was purified by flash chromatography using 100-200 mesh silica gel and 2% EtOAc in pet ether as an eluent to afford 4-bromo-2-(bromomethyl)-1-nitrobenzene as a pale yellow solid (13.9 g, 49%).

Step 2: Diethyl 2-acetamidomalonate (9.66 g, 44.55 mmol) was treated with a solution of freshly prepared NaOEt solution (1.06 g of Na metal was dissolved in in 120 mL EtOH at 0° C. under a nitrogen atmosphere) at RT and stirred for 1 h. The resulting reaction mixture was treated with 1-bromo-2-(bromomethyl)-3-nitrobenzene (11.9 g, 40.5 mmol) and KI (0.67 g, 4.05 mmol) at 60° C. and stirred at 60° C. for 2.5 h. Progress of the reaction was monitored by TLC. TLC mobile phase: 30% EtOAc in pet ether, RF: 0.2, TLC detection: UV. The reaction mixture was diluted with ice water (150 mL) and the product was extracted with EtOAc (3×100 mL). The organic layer was dried over Na₂SO₄ and evaporated under reduced pressure to afford crude product to afford diethyl 2-acetamido-2-(5-bromo-2-nitrobenzyl)malonate as yellow solid (12.9 g, 62%). (LC/MS; m/z 431.1 [M+H]⁺). The product was used without further purification in the next step.

Step 3: A sat. NH₄Cl solution (12.6 mL) and iron powder (3.11 g, 56 mmol) were added to a solution of diethyl 2-acetamido-2-(5-bromo-2-nitrobenzyl)malonate (6.0 g, 14.0 mmol) in EtOH (40 mL) and THF (20 mL) at RT. The reaction mixture was heated to reflux for 3.5 h. Progress of the reaction was monitored by TLC. TLC mobile phase: 50% EtOAc in pet ether, RF: 0.3, TLC detection: UV. The reaction mixture was filtered through a celite pad and washed several times with MeOH (60 mL). The filtrate was evaporated, diluted with EtOAc (100 mL) and washed with water (3×50 mL). The organic layer was dried over Na₂SO₄ and evaporated under reduced pressure to afford ethyl 3-acetamido-6-bromo-2-oxo-1,2,3,4-tetrahydroquinoline-3-carboxylate as a yellow solid (5.0 g, 96%). (LC/MS; m/z 355.1 [M+H]⁺). The product was used without further purification in the next step.

Step 4: Ethyl 3-acetamido-6-bromo-2-oxo-1,2,3,4-tetrahydroquinoline-3-carboxylate (5 g, 14.1 mmol, LC/MS 95%) was dissolved in conc. HCl (50 mL) and heated at 120° C. for 16 h. Progress of the reaction was monitored by TLC. TLC mobile phase: 50% EtOAc in pet ether. The reaction mixture was cooled to RT, solid material was filtered, washed with cold water (100 mL) and dried under reduced pressure to afford 3-amino-6-bromo-3,4-dihydroquinolin-2(1H)-one hydrochloride as a pale yellow solid (2.2 g, 56%). (LC/MS; m/z 241.2 [M+H]⁺). The product was used without further purification in the next step.

Step 5: A solution of 3-amino-6-bromo-3,4-dihydroquinolin-2(1H)-one hydrochloride (2.2 g, 8 mmol, LC/MS 94%) in THF (17 mL) was cooled to 0° C., treated with TEA (2.0 mL, 20 mmol) followed by a solution of (Boc)₂O (1.74 mL, 8.0 mmol) in THF (5 mL) under an argon atmosphere. The solution was stirred at RT for 1 h. The reaction was monitored by TLC. TLC mobile phase: 20% EtOAc in pet ether, RF: 0.53, TLC detection: UV. The reaction mixture was diluted with cold water (40 mL) and extracted with EtOAc (3×40 mL). The organic layer was separated, dried over Na₂SO₄ and evaporated under reduced pressure to afford crude product (2.6 g) which was purified by flash chromatography (Grace) using a 12 g reveleris column (silica gel) and 11% EtOAc in pet ether gradient to afford tert-butyl (6-bromo-2-oxo-1,2,3,4-tetrahydroquinolin-3-yl)carbamate as an off-white solid (2.2 g, 86%). (LC/MS; m/z 341.1 [M+H]⁺)

Step 6: A solution of tert-butyl (6-bromo-2-oxo-1,2,3,4-tetrahydroquinolin-3-yl)carbamate (2.2 g, 6.5 mmol) in DCM (25 mL) was treated with (4-(trifluoromethyl)phenyl)boronic acid (2.47 g, 13 mmol), Cu(OAc)₂ (2.3 g, 13 mmol) and DIPEA (5.8 mL, 32.3 mmol). The reaction mixture was stirred under an oxygen atmosphere (balloon pressure) at RT for 24 h. Progress of the reaction was monitored by TLC. TLC mobile phase: 20% EtOAc in pet ether, RF: 0.5, TLC detection: UV active. The reaction mixture was filtered through a celite pad washed with EtOAc (60 mL). The filtrate was evaporated under reduced pressure to afford crude product as a pale yellow gum (3 g, LC/MS 28%) which was purified by normal phase chromatography (Grace) using a 24 g column and eluted with 12% EtOAc in pet ether gradient to afford tert-butyl (6-bromo-2-oxo-1-(4-(trifluoromethyl)phenyl)-1,2,3,4-tetrahydroquinolin-3-yl)carbamate as an off-white solid (1 g, 21%). (LC/MS; m/z 485.3 [M+H]⁺)

Step 7: A solution of tert-butyl (6-bromo-2-oxo-1-(4-(trifluoromethyl)phenyl)-1,2,3,4-tetrahydroquinolin-3-yl)carbamate (1.0 g, 2.1 mmol, LC/MS 69%) in THF (10 mL) was cooled to 0° C. and treated with BH₃·THF (1M in THF, 10.5 mL, 10.5 mmol) under a nitrogen atmosphere. The reaction mixture was stirred at RT for 3 h. Progress of the reaction was monitored by TLC. TLC mobile phase: 20% EtOAc in pet ether, RF: 0.5, TLC detection: UV. The reaction mixture was cooled to 0° C., quenched with MeOH (10 mL) and evaporated under reduced pressure to afford crude product (750 mg, LC/MS 56%) which was purified by flash chromatography (Grace) using a 40 g reveleris column and eluted with 40% EtOAc in pet ether gradient to afford tert-butyl (6-bromo-1-(4-(trifluoromethyl)phenyl)-1,2,3,4-tetrahydroquinolin-3-yl)carbamate (Cpd. No. 057) as a yellow solid (361 mg, 54%). (LC/MS; m/z 471.2 [M+H]⁺)

### Examples 24-25: Synthesis of N-(5-bromo-1-(4-(trifluoromethyl)phenyl)-1,2,3,4-tetrahydroquinolin-3-yl)acrylamide (Cpd. No. 058) and N-(5-(pyridin-3-ylmethyl)-1-(4-(trifluoromethyl)phenyl)-1,2,3,4-tetrahydroquinolin-3-yl)acrylamide (Cpd. No. 059)

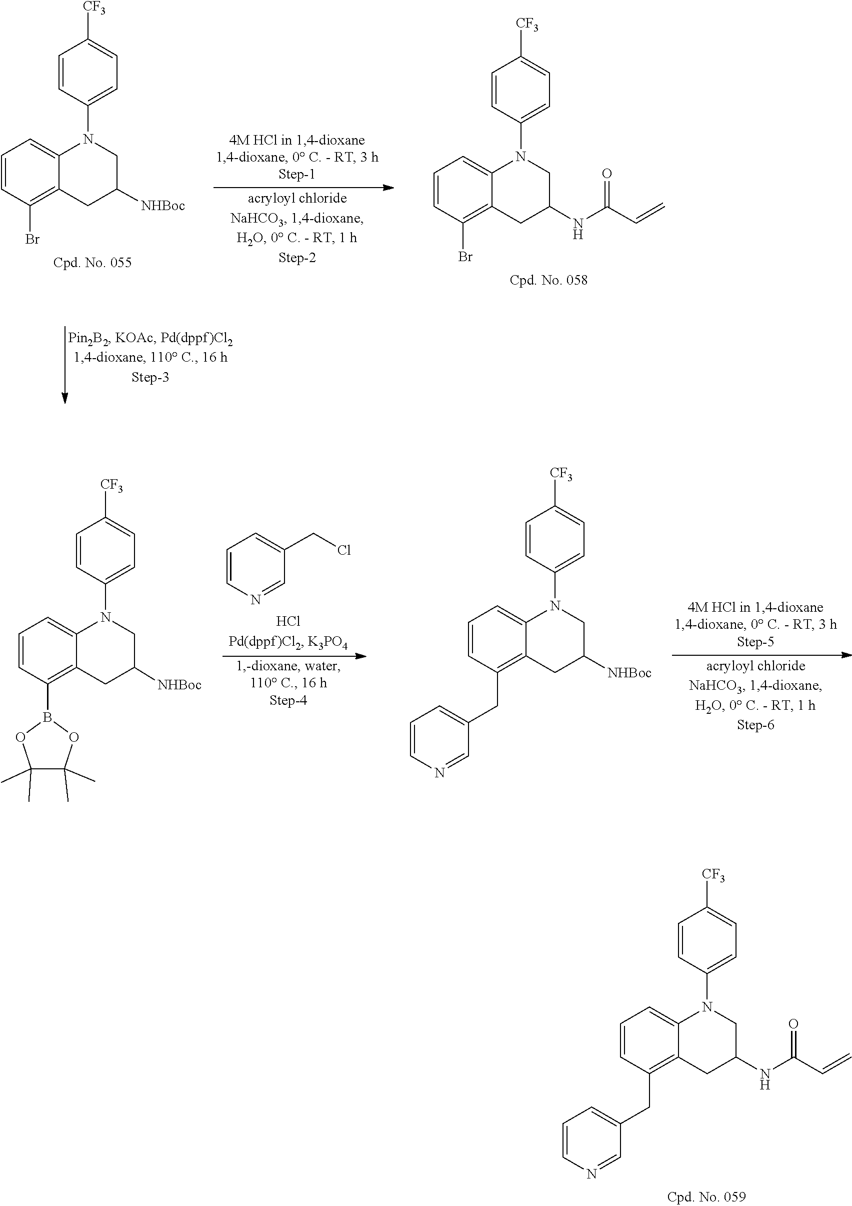 [SMILES: CC(C)(C)OC(=O)NC1Cc2c(Br)cccc2N(c2ccc(C(F)(F)F)cc2)C1 ; C=CC(=O)NC1Cc2c(Br)cccc2N(c2ccc(C(F)(F)F)cc2)C1 ; CC(C)(C)OC(=O)NC1Cc2c(B3OC(C)(C)C(C)(C)O3)cccc2N(c2ccc(C(F)(F)F)cc2)C1 ; ClCc1cccnc1 ; CC(C)(C)OC(=O)NC1Cc2c(Cc3cccnc3)cccc2N(c2ccc(C(F)(F)F)cc2)C1 ; C=CC(=O)NC1Cc2c(Cc3cccnc3)cccc2N(c2ccc(C(F)(F)F)cc2)C1]

Steps 1-2: These steps were executed in a manner similar (use of appropriate reagents and purification methods known to the person skilled in the art) to Cpd. No. 001. Starting material (Cpd. No. 055; 300 mg, 0.63 mmol) yielded a yellow solid which was purified by preparative HPLC method H8. The collected fractions were concentrated under reduced pressure and lyophilised to afford N-(5-bromo-1-(4-(trifluoromethyl)phenyl)-1,2,3,4-tetrahydroquinolin-3-yl)acrylamide (Cpd. No. 058) as an off-white solid (72 mg, 23%). (LC/MS; m/z 425.2 [M+H]⁺)

Step 3: A solution of tert-butyl (5-bromo-1-(4-(trifluoromethyl)phenyl)-1,2,3,4-tetrahydroquinolin-3-yl)carbamate (Cpd. No. 055) (1 g, 2.1 mmol, LC/MS 99%) in 1,4-dioxane (10 mL) was treated with Pin₂B₂(0.8 g, 3.15 mmol) and KOAc (0.515 g, 5.25 mmol) at RT. The reaction mixture was degassed with argon for 15 min, treated with Pd(dppf)Cl₂ (0.153 g, 0.21 mmol) and stirred at 110° C. for 16 h. The reaction progress was monitored by TLC. TLC mobile phase: 10% EtOAc in pet ether, RF: 0.29, TLC detection: UV. The reaction mixture was cooled to RT, filtered through a celite pad and rinsed with EtOAc (100 mL). The filtrate was washed with water (100 mL), dried over Na₂SO₄ and concentrated to afford crude product as a pale brown gum (1.3 g, LC/MS 69%) which was purified by normal phase column chromatography (Combi flash) using a 24 g column and a gradient of 7% EtOAc in pet ether as an eluent to afford tert-butyl (5-(4,4,5,5-tetramethyl-1,3,2-dioxaborolan-2-yl)-1-(4-(trifluoromethyl)phenyl)-1,2,3,4-tetrahydroquinolin-3-yl)carbamate as a pale yellow solid (800 mg, 61%). (LC/MS; m/z 519.3 [M+H]⁺)

Step 4: A solution of tert-butyl (5-(4,4,5,5-tetramethyl-1,3,2-dioxaborolan-2-yl)-1-(4-(trifluoromethyl)phenyl)-1,2,3,4-tetrahydroquinolin-3-yl)carbamate (580 mg, 1.119 mmol, LC/MS 84%) (sealed tube) in 1,4-dioxane (6 mL) and water (3 mL) was treated with 3-(chloromethyl)pyridine hydrochloride (200.7 mg, 1.231 mmol) and K₃PO₄ (949.5 mg, 4.478 mmol) at RT. The reaction mixture was degassed with argon for 15 min, treated with Pd(dppf)Cl₂ (81.9 mg, 0.111 mmol) and stirred at 110° C. for 6 h. The reaction progress was monitored by TLC. TLC mobile phase: 50% EtOAc in pet ether, RF: 0.20, TLC detection: UV. The reaction mixture was cooled to RT, filtered through a celite pad and rinsed with EtOAC (50 mL). The filtrate was washed with water (50 mL), dried over Na₂SO₄ and concentrated to afford crude product as a brown semi solid (700 mg, LC/MS 76%) which was purified by normal phase column chromatography (Grace) using 24 g column and a gradient of 35% EtOAc in pet ether as an eluent to afford tert-butyl (5-(pyridin-3-ylmethyl)-1-(4-(trifluoromethyl)phenyl)-1,2,3,4-tetrahydroquinolin-3-yl)carbamate as an off-white solid (380 mg, 83%). (LC/MS; m/z 484.2 [M+H]⁺)

Steps 5-6: These steps were executed in a manner similar (use of appropriate reagents and purification methods known to the person skilled in the art) to Cpd. No. 001. Starting material (380 mg, 0.786 mmol) yielded a pale brown gum which was purified by preparative HPLC method H5. The collected fractions were concentrated under reduced pressure and lyophilised to afford N-(5-(pyridin-3-yl-methyl)-1-(4-(trifluoromethyl)phenyl)-1,2,3,4-tetrahydroquinolin-3-yl)acrylamide (Cpd. No. 059) as an off-white solid (29 mg, 18%). (LC/MS; m/z 438.2 [M+H]⁺)

The following compounds were prepared in a manner similar to Cpd. No. 058 and Cpd. No. 059 by using appropriate reagents and purification methods known to the person skilled in the art: Cpd. No. 060, Cpd. No. 061, Cpd. No. 062, and Cpd. No. 063. Cpd. No. 060 is prepared from Cpd. No. 056.

### Example 26: Synthesis of N-(5-methyl-1-(4-(trifluoromethyl)phenyl)-1,2,3,4-tetrahydroquinolin-3-yl)acrylamide (Cpd. No. 064)

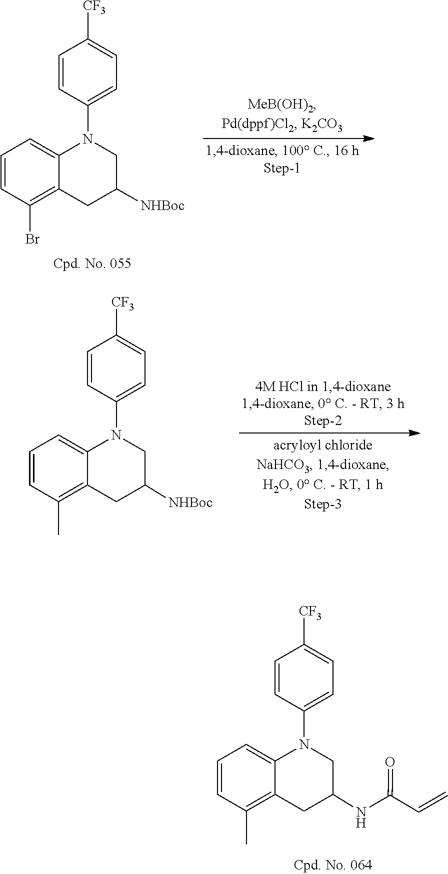 [SMILES: CC(C)(C)OC(=O)NC1Cc2c(Br)cccc2N(c2ccc(C(F)(F)F)cc2)C1 ; Cc1cccc2c1CC(NC(=O)OC(C)(C)C)CN2c1ccc(C(F)(F)F)cc1 ; C=CC(=O)NC1Cc2c(C)cccc2N(c2ccc(C(F)(F)F)cc2)C1]

Step 1: A stirred solution of tert-butyl (5-bromo-1-(4-(trifluoromethyl)phenyl)-1,2,3,4-tetrahydroquinolin-3-yl)carbamate (Cpd. No. 055) (400 mg, 0.82 mmol) in 1,4-dioxane (4 mL) was treated with K₂CO₃ (285 mg, 2.0 mmol), methyl boronic acid (59.4 mg, 0.9 mmol) and degassed with argon for 5 min. Pd(dppf)Cl₂ (60.4 mg, 0.08 mmol) was added to the reaction mixture and stirred at 100° C. for 16 h. Progress of the reaction was monitored by TLC. TLC mobile phase: 10% EtOAc in pet ether, RF: 0.3, TLC detection: UV. The reaction mixture was diluted with cold water (20 mL) and extracted with EtOAc (2×10 mL). The organic layer was separated, dried over Na₂SO₄, filtered and the filtrate was evaporated under reduced pressure to afford crude product (520 mg) which was purified by flash chromatography (Grace) using a 40 g reveleris column and eluted with 15% EtOAc in pet ether gradient to afford tert-butyl (5-methyl-1-(4-(trifluoromethyl)phenyl)-1,2,3,4-tetrahydroquinolin-3-yl)carbamate as an off-white solid (220 mg, 49%). (LC/MS; m/z 407.1 [M+H]⁺)

Steps 2-3: These steps were executed in a manner similar (use of appropriate reagents and purification methods known to the person skilled in the art) to Cpd. No. 001. Starting material (220 mg, 0.54 mmol, LC/MS 77%) yielded a yellow liquid which was purified by preparative HPLC method H5. The collected fractions were concentrated under reduced pressure and lyophilised to afford N-(5-methyl-1-(4-(trifluoromethyl)phenyl)-1,2,3,4-tetrahydroquinolin-3-yl)acrylamide (Cpd. No. 064) as an off-white solid (40 mg, 32%). (LC/MS; m/z 361.2 [M+H]⁺)

The following compounds were prepared in a manner similar to Cpd. No. 064 by using appropriate reagents and purification methods known to the person skilled in the art: Cpd. No. 065, Cpd. No. 066, Cpd. No. 067, and Cpd. No. 068. Cpd. No. 065 was prepared starting from Cpd. No. 057. To prepare Cpd. No. 066 and Cpd. No. 067, 2-benzyl-4,4,5,5-tetramethyl-1,3,2-dioxaborolane and K₃PO₄ in water and 1,4-dioxane were employed. Similar conditions and 4-((4,4,5,5-tetramethyl-1,3,2-dioxaborolan-2-yl)methyl)morpholine were employed to prepare Cpd. No. 068.

### Examples 27-28: Synthesis of N-(5-(phenylamino)-1-(4-(trifluoromethyl)phenyl)-1,2,3,4-tetrahydroquinolin-3-yl)acrylamide (Cpd. No. 069) and N-(5-(pyridin-3-ylamino)-1-(4-(trifluoromethyl)phenyl)-1,2,3,4-tetrahydroquinolin-3-yl)acrylamide (Cpd. No. 070)

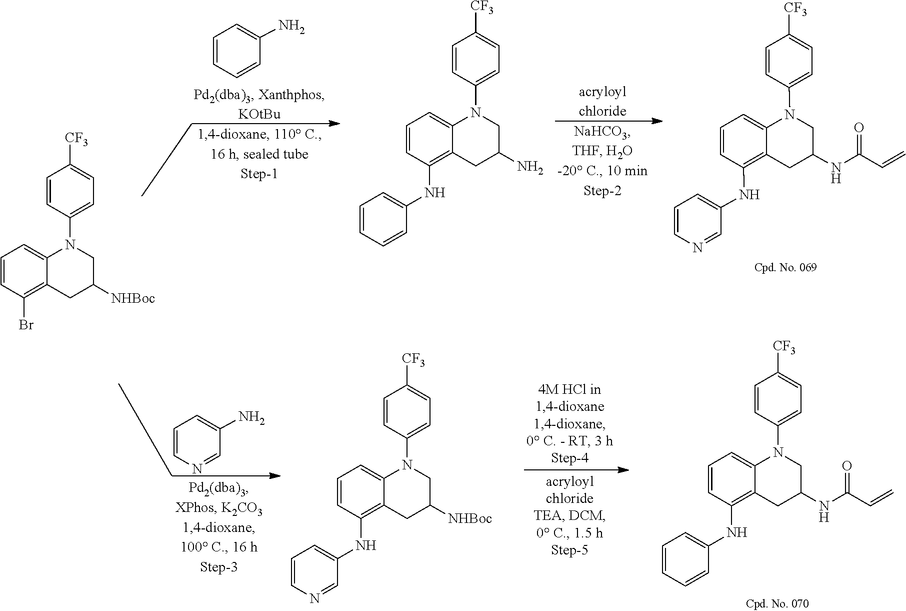

Step 1: A solution of tert-butyl (5-bromo-1-(4-(trifluoromethyl)phenyl)-1,2,3,4-tetrahydroquinolin-3-yl)carbamate (Cpd. No. 055) (800 mg, 1.70 mmol) in 1,4-dioxane (10 mL) (sealed tube) was treated with KOtBu (476 mg, 4.25 mmol) and aniline (474 mg, 5.10 mmol). The reaction mixture was degassed with argon for 10 min, treated with Xantphos (196 mg, 0.34 mmol), Pd₂(dba)₃ (155 mg, 0.17 mmol) and stirred at 110° C. for 16 h. Progress of the reaction mixture was monitored by TLC. TLC mobile phase: 20% EtOAc in pet ether, RF: 0.04, TLC detection: UV. The reaction mixture was cooled at RT, filtered through a celite pad and rinsed with EtOAc (50 mL). The filtrate was washed with water (2×50 mL), dried over Na₂SO₄ and evaporated under reduced pressure to afford crude product as a pale brown gum (900 mg, LC/MS 69%) which was purified by normal phase chromatography (GRACE) using a 24 g column and a gradient of EtOAc as an eluent to afford N⁵-phenyl-1-(4-(trifluoromethyl)phenyl)-1,2,3,4-tetrahydroquinoline-3,5-diamine as a yellow solid (450 mg, 66%). (LC/MS; m/z 384.2 [M+H]⁺)

Step 2: This step was executed in a manner similar (use of appropriate reagents and purification methods known to the person skilled in the art) to Cpd. No. 001. Starting material (100 mg, 0.26 mmol, LC/MS 96%) yielded a light blue solid which was purified by preparative HPLC method H5. The collected fractions were concentrated under reduced pressure and lyophilised to afford N-(5-(phenylamino)-1-(4-(trifluoromethyl)phenyl)-1,2,3,4-tetrahydroquinolin-3-yl)acrylamide (Cpd. No. 069) as an off-white solid (28 mg, 25%). (LC/MS; m/z 438.2 [M+H]⁺)

Step 3: A solution of tert-butyl (5-bromo-1-(4-(trifluoromethyl)phenyl)-1,2,3,4-tetrahydroquinolin-3-yl)carbamate (Cpd. No. 055) (500 mg, 1.06 mmol) in 1,4-dioxane (5 mL) (sealed tube) was treated with K₂CO₃ (440 mg, 3.1 mmol) and degassed with argon for 5 min. Pyridin-3-amine (149 mg, 1.5 mmol), XPhos (99.9 mg, 0.21 mmol), Pd₂(dba)₃ (97.3 mg, 0.1 mmol) were added to the reaction mixture under an argon atmosphere and stirred at 100° C. for 16 h. Progress of the reaction was monitored by TLC. TLC mobile phase: 50% EtOAc in pet ether, RF: 0.17, TLC detection: UV.

The reaction mixture was diluted with ice water (15 mL) and extracted with EtOAc (2×8 mL). The organic layer was dried over Na₂SO₄, filtered and the filtrate was evaporated under reduced pressure to afford crude product (800 mg) which was purified by flash chromatography using a 40 g reveleris column and eluted with 50% EtOAc in pet ether gradient to afford tert-butyl (5-(pyridin-3-ylamino)-1-(4-(trifluoromethyl)phenyl)-1,2,3,4-tetrahydroquinolin-3-yl)carbamate as a brown solid (240 mg, 46%). (LC/MS; m/z 485.4 [M+H]⁺)

Steps 4-5: These steps were executed in a manner similar (use of appropriate reagents and purification methods known to the person skilled in the art) to Cpd. No. 001. Starting material (190 mg, 0.39 mmol) yielded a brown liquid which was purified by preparative HPLC method H5. The collected fractions were concentrated under reduced pressure and lyophilised to afford N-(5-(pyridin-3-ylamino)-1-(4-(trifluoromethyl)phenyl)-1,2,3,4-tetrahydroquinolin-3-yl)acrylamide (Cpd. No. 070) as an off-white solid (26 mg, 18%). (LC/MS; m/z 439.2 [M+H]⁺)

The following compounds were prepared in a manner similar to Cpd. No. 069 and Cpd. No. 070, respectively by using appropriate reagents and purification methods known to the person skilled in the art: Cpd. No. 071 and Cpd. No. 072.

### Example 29: Synthesis of N-(5-methoxy-1-(4-(trifluoromethyl)phenyl)-1,2,3,4-tetrahydroquinolin-3-yl)acrylamide (Cpd. No. 073)

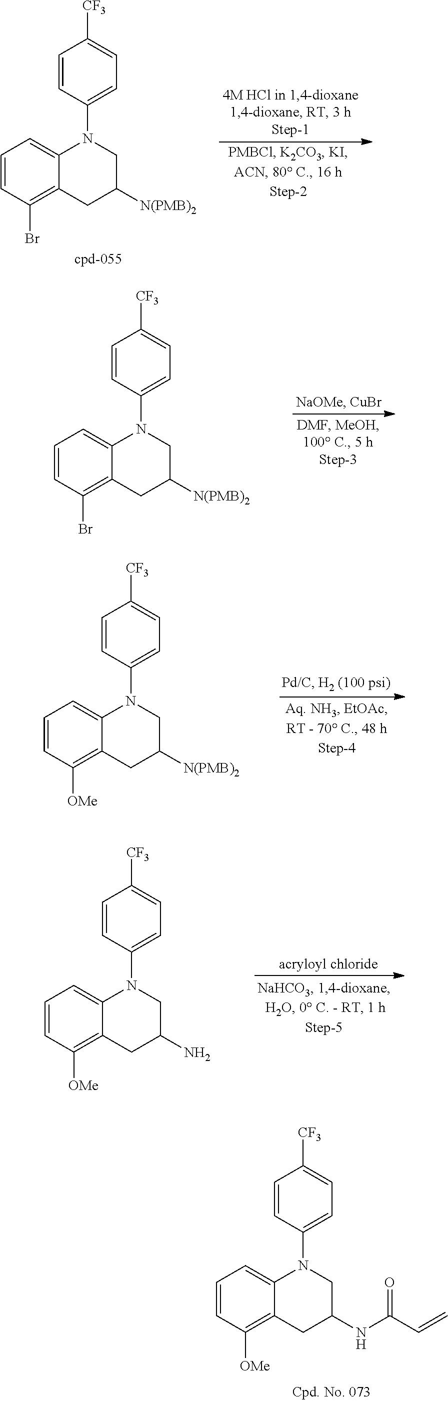

Steps 1-2: A solution of tert-butyl (5-bromo-1-(4-(trifluoromethyl)phenyl)-1,2,3,4-tetrahydroquinolin-3-yl)carbamate (Cpd. No. 055) (2.0 g, 4.3 mmol, LC/MS 82%) in 1,4-dioxane (8 mL) was dissolved with HCl (4M in 1,4-dioxane, 20 mL) at 0° C. under a nitrogen atmosphere. The reaction mixture was stirred at RT for 3 h. Progress of the reaction was monitored by TLC. TLC mobile phase: 20% EtOAc in pet ether. The reaction mixture was evaporated under reduced pressure, obtained residue was washed with Et₂O (15 mL) and dried to afford 5-bromo-1-(4-(trifluoromethyl)phenyl)-1,2,3,4-tetrahydroquinolin-3-amine hydrochloride as a yellow solid (1.5 g, 99%). (LC/MS; m/z 372.9 [M+H]⁺). A solution of 5-bromo-1-(4-(trifluoromethyl)phenyl)-1,2,3,4-tetrahydroquinolin-3-amine hydrochloride (1.5 g, 3.7 mmol, LC/MS 94%) in ACN (20 mL) was treated with KI (61 mg, 0.37 mmol), K₂CO₃ (1.53 g, 11.1 mmol) and PMBCI (0.86 g, 5.55 mmol) at 0° C. under a nitrogen atmosphere. The reaction mixture was stirred at 80° C. for 16 h. Progress of the reaction was monitored by TLC. TLC mobile phase: 20% EtOAc in pet ether, RF: 0.6, TLC detection: UV. The reaction mixture was diluted with ice water (60 mL) and extracted with EtOAc (2×20 mL). The organic layer was dried over Na₂SO₄, filtered and filtrate was evaporated under reduced pressure to afford crude product (3.0 g) which was purified by flash chromatography using a 40 g reveleris column and eluted with 20% EtOAc in pet ether gradient to afford 5-bromo-N,N-bis(4-methoxybenzyl)-1-(4-(trifluoromethyl)phenyl)-1,2,3,4-tetrahydroquinolin-3-amine as a yellow gum (2.1 g, 89%). (LC/MS; m/z 611.0 [M+H]⁺)

Step 3: A solution of freshly prepared NaOMe solution (48 mg of Na metal was dissolved in 10 mL of MeOH) was treated with 5-bromo-N,N-bis(4-methoxybenzyl)-1-(4-(trifluoromethyl)phenyl)-1,2,3,4-tetrahydroquinolin-3-amine (1.2 g, 2.0 mmol, LC/MS 90%), CuBr (28 mg, 0.2 mmol) and DMF (4 mL). The reaction mixture was stirred at 100° C. for 16 h. Progress of the reaction was monitored by TLC. TLC mobile phase: 20% EtOAc in pet ether, RF: 0.4, TLC detection: UV. The reaction mixture was diluted with ice water (20 mL) and extracted with EtOAc (2×10 mL). The organic layer was dried over Na₂SO₄, filtered and the filtrate was evaporated under reduced pressure to afford crude product (2.0 g) which was purified by flash chromatography using a 24 g reveleris column and eluted with 20% EtOAc in pet ether gradient to afford 5-methoxy-N,N-bis(4-methoxybenzyl)-1-(4-(trifluoromethyl)phenyl)-1,2,3,4-tetrahydroquinolin-3-amine as a yellow gum (720 mg, 63%). (LC/MS; m/z 562.7 [M+H]⁺)

Step 4: To a solution of 5-methoxy-N,N-bis(4-methoxybenzyl)-1-(4-(trifluoromethyl)phenyl)-1,2,3,4-tetrahydroquinolin-3-amine (400 mg, 0.71 mmol, LC/MS 87%) in EtOAc (5 mL) was added 10% Pd/C (120 mg) and catalytic aq. NH₃ (1.5 mL). The solution was put under a hydrogen gas atmosphere (100 psi) for 48 h at 70° C. Progress of the reaction was monitored by TLC. TLC mobile phase: 30% EtOAc in pet ether. The reaction mixture was filtered through a celite pad and washed with EtOAc (15 mL). The filtrate was evaporated under reduced pressure to afford 5-methoxy-1-(4-(trifluoromethyl) phenyl)-1,2,3,4-tetrahydroquinolin-3-amine as a yellow liquid (185 mg, 87%). (LC/MS; m/z 322.9 [M+H]⁺)

Step 5: This step was executed in a manner similar (use of appropriate reagents and purification methods known to the person skilled in the art) to Cpd. No. 001. Starting material (336 mg, 1.03 mmol) yielded a brown solid which was purified by preparative HPLC method H5. The collected fractions were concentrated under reduced pressure and lyophilised to afford N-(5-methoxy-1-(4-(trifluoromethyl)phenyl)-1,2,3,4-tetrahydroquinolin-3-yl)acrylamide (Cpd. No. 073) as an off-white solid (14 mg, 4%). (LC/MS; m/z 377.1 [M+H]⁺)

### Examples 30-32: Synthesis of tert-butyl (3-acrylamido-1-(4-(trifluoromethyl)phenyl)-1,2,3,4-tetrahydroquinolin-5-yl)carbamate (Cpd. No. 074), N-(5-amino-1-(4-(trifluoromethyl)phenyl)-1,2,3,4-tetrahydroquinolin-3-yl)acrylamide (Cpd. No. 075), and N-(5-acetamido-1-(4-(trifluoromethyl)phenyl)-1,2,3,4-tetrahydroquinolin-3-yl)acrylamide (Cpd. No. 076)

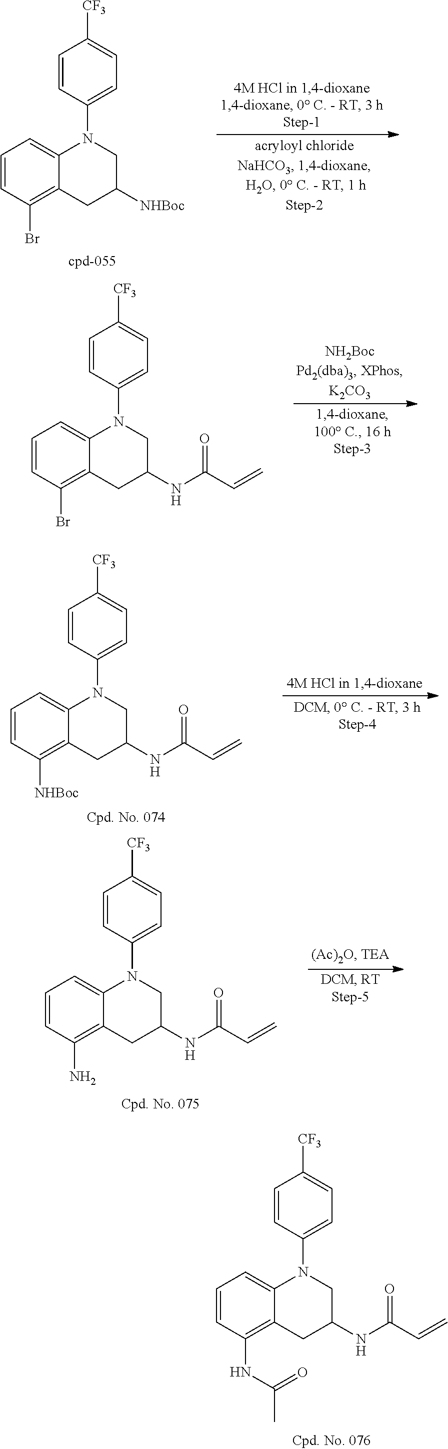 [SMILES: CC(C)(C)OC(=O)NC1Cc2c(Br)cccc2N(c2ccc(C(F)(F)F)cc2)C1 ; C=CC(=O)NC1Cc2c(Br)cccc2N(c2ccc(C(F)(F)F)cc2)C1 ; C=CC(=O)NC1Cc2c(NC(=O)OC(C)(C)C)cccc2N(c2ccc(C(F)(F)F)cc2)C1 ; C=CC(=O)NC1Cc2c(N)cccc2N(c2ccc(C(F)(F)F)cc2)C1 ; C=CC(=O)NC1Cc2c(NC(C)=O)cccc2N(c2ccc(C(F)(F)F)cc2)C1]

Steps 1-2: These steps were executed in a manner similar (use of appropriate reagents and purification methods known to the person skilled in the art) to Cpd. No. 001. Starting material (Cpd. No. 055; 2.3 g, 4.9 mmol) yielded N-(5-bromo-1-(4-(trifluoromethyl)phenyl)-1,2,3,4-tetrahydroquinolin-3-yl)acrylamide as a brown solid (900 mg, 100%). (LC/MS; m/z 425.2 [M+H]⁺)

Step 3: A solution of N-(5-bromo-1-(4-(trifluoromethyl)phenyl)-1,2,3,4-tetrahydroquinolin-3-yl)acrylamide (900 mg, 2.12 mmol, LC/MS 82%) in 1,4-dioxane (10 mL) (sealed tube) was treated with K₂CO₃ (878 mg, 6.3 mmol) and NH₂Boc (372 mg, 3.1 mmol). The reaction mixture was degassed with argon for 5 min, treated with XPhos (199 mg, 0.42 mmol), Pd₂(dba)₃ (194 mg, 0.2 mmol) and stirred at 100° C. for 16 h. Progress of the reaction was monitored by TLC. TLC mobile phase: 30% EtOAc in pet ether, RF: 0.3, TLC detection: UV. The reaction mixture was diluted with ice water (30 mL) and extracted with EtOAc (3×10 mL). The organic layer was dried over Na₂SO₄ filtered and the filtrate was evaporated under reduced pressure to afford crude product as a yellow liquid (1.2 g, LC/MS 10%) which was purified by normal phase chromatography (Grace) using a 40 g reveleris column and a gradient of 30% EtOAc in pet ether to afford the product (200 mg, LC/MS 62%). It was further purified by preparative HPLC method H43. The collected fractions were lyophilised to afford tert-butyl (3-acrylamido-1-(4-(trifluoromethyl)phenyl)-1,2,3,4-tetrahydroquinolin-5-yl)carbamate (Cpd. No. 074) as an off-white solid (35 mg, 4%). (LC/MS; m/z 462.2 [M+H]⁺)

Step 4: A solution of tert-butyl (3-acrylamido-1-(4-(trifluoromethyl)phenyl)-1,2,3,4-tetrahydroquinolin-5-yl)carbamate (Cpd. No. 074) (800 mg, 1.73 mmol, LC/MS 82%) in 1,4-dioxane (2 mL) was treated with HCl (4M in 1,4-dioxane, 8 mL) at 0° C. under a nitrogen atmosphere. The reaction mixture was stirred at RT for 3 h. Progress of the reaction was monitored by TLC. TLC mobile phase: 30% EtOAc in pet ether. The reaction mixture was evaporated under reduced pressure to afford N-(5-amino-1-(4-(trifluoromethyl)phenyl)-1,2,3,4-tetrahydroquinolin-3-yl)acrylamide hydrochloride (Cpd. No. 075·HCl) as a yellow solid (500 mg, LC/MS 53%). 250 mg of the crude compound was basified with aq. NaHCO₃ and further purified by preparative HPLC method H3. The collected fractions were evaporated under lyophilisation to afford N-(5-amino-1-(4-(trifluoromethyl)phenyl)-1,2,3,4-tetrahydroquinolin-3-yl)acrylamide (Cpd. No. 075) as an off-white solid (40 mg, 33%). (LC/MS; m/z 362.2 [M+H]⁺) Chiral SFC purification: 120 mg of Cpd. No. 075 was purified by preparative SFC method K5 to afford Cpd. No. 075-En1 (28 mg) and Cpd. No. 075-En2 (30 mg), both as an off-white solid. (LC/MS; m/z 431.3 [M+H]⁺) The chiral purity of both enantiomers was assessed by analytic SFC method S5: Cpd. No. 075-En1, 99.1% ee; Cpd. No. 075-En2, 97.4% ee.

Step 5: A solution of N-(5-amino-1-(4-(trifluoromethyl)phenyl)-1,2,3,4-tetrahydroquinolin-3-yl)acrylamide hydrochloride (Cpd. No. 075·HCl) (150 mg, 0.37 mmol, LC/MS 53%) in DCM (5 mL) was cooled to 0° C., treated with TEA (114 mg, 1.13 mmol), Ac₂O (46 mg, 0.4 mmol) under a nitrogen atmosphere and stirred for 1 h at RT. Progress of the reaction was monitored by TLC. TLC mobile phase: 50% EtOAc in pet ether, RF: 0.34, TLC detection: UV. The reaction mixture was diluted with ice water (15 mL) and extracted with DCM (2×10 mL). The organic layer was separated and dried over Na₂SO₄ and concentrated under reduced pressure to afford crude product as a brown solid (130 mg, LC/MS 51%) which was purified by preparative HPLC method H2. The collected fractions were evaporated under lyophilisation to afford N-(5-acetamido-1-(4-(trifluoromethyl)phenyl)-1,2,3,4-tetrahydroquinolin-3-yl)acrylamide (Cpd. No. 076) as an off-white solid (33 mg, 40%). (LC/MS; m/z 404.2 [M+H]⁺)

### Example 33: Synthesis of N-(5-((2-methoxyethyl)amino)-1-(4-(trifluoromethyl)phenyl)-1,2,3,4-tetrahydroquinolin-3-yl)acrylamide (Cpd. No. 077)

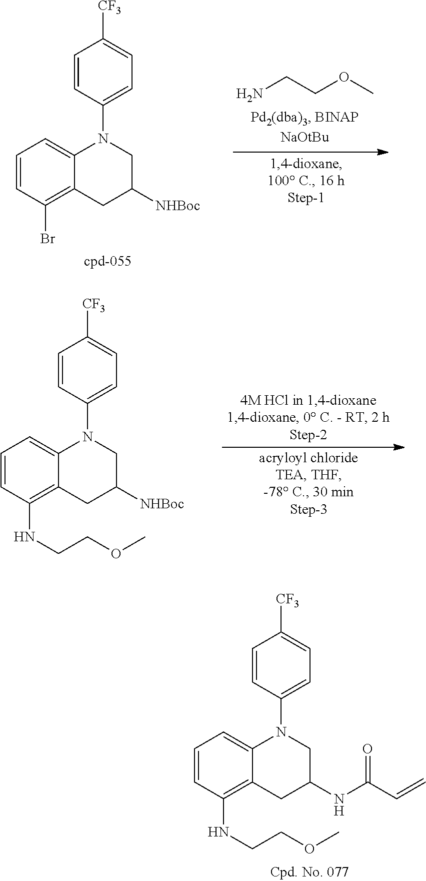

Step 1: A solution of tert-butyl (5-bromo-1-(4-(trifluoromethyl)phenyl)-1,2,3,4-tetrahydroquinolin-3-yl)carbamate (Cpd. No. 055) (500 mg, 1.06 mmol, LC/MS 92%) and 2-methoxyethan-1-amine (199.5 mg, 2.66 mmol) in 1,4-dioxane (10 mL) in a sealed tube was treated with NaOtBu (357.4 mg, 3.72 mmol) and degassed with argon for 10 min. BINAP (132.3 mg, 0.21 mmol) and Pd₂(dba)₃ (97.4 mg, 0.11 mmol) were added to the reaction mixture, degassed for another 5 min and stirred at 100° C. for 16 h. Progress of the reaction was monitored by TLC. TLC mobile phase: 30% EtOAc in pet ether, RF: 0.3, TLC detection: UV. The reaction mixture was cooled to RT, filtered through a celite pad and washed with EtOAc (80 mL). The filtrate was concentrated under reduced pressure to afford crude product (600 mg, LC/MS 48%) which was purified by normal phase chromatography (Combi) using a 12 g reveleris column and a gradient of 15% EtOAc in pet ether as an eluent to afford tert-butyl (5-((2-methoxyethyl)amino)-1-(4-(trifluoromethyl)phenyl)-1,2,3,4-tetrahydroquinolin-3-yl)carbamate as a light brown solid (335 mg, 63%). (LC/MS; m/z 466.4 [M+H]⁺)

Steps 2-3: These steps were executed in a manner similar (use of appropriate reagents and purification methods known to the person skilled in the art) to Cpd. No. 001. Starting material (400 mg, 0.86 mmol) yielded crude product (381 mg) which was purified by preparative HPLC method H3. The collected fractions were concentrated under reduced pressure and lyophilised to afford N-(5-((2-methoxyethyl)amino)-1-(4-(trifluoromethyl)phenyl)-1,2,3,4-tetrahydro-quinolin-3-yl)acrylamide (Cpd. No. 077) as a white solid (25 mg, 10%). (LC/MS; m/z 420.3 [M+H]⁺)

### Examples 34-35: Synthesis of N-(5-cyano-1-(4-(trifluoromethyl)phenyl)-1,2,3,4-tetrahydroquinolin-3-yl)acrylamide (Cpd. No. 078) and 3-acrylamido-1-(4-(trifluoromethyl)phenyl)-1,2,3,4-tetrahydroquinoline-5-carboxylic acid (Cpd. No. 079)

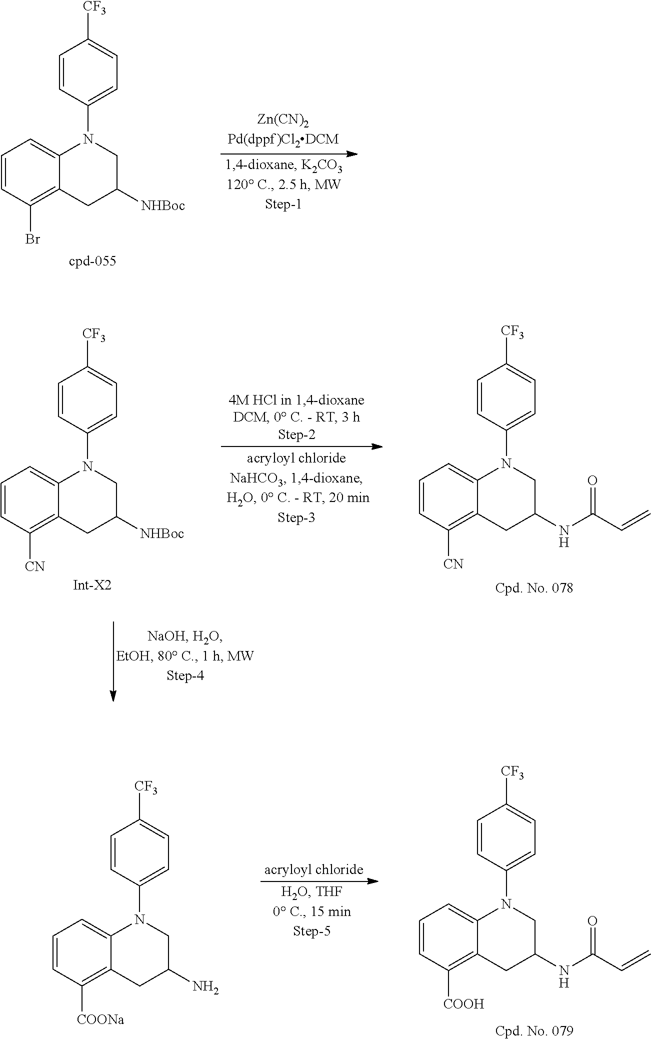

Step 1: A solution of tert-butyl (5-bromo-1-(4-(trifluoromethyl)phenyl)-1,2,3,4-tetrahydroquinolin-3-yl)carbamate (Cpd. No. 055) (500 mg, 1.063 mmol) in 1,4-dioxane (5.0 mL) was treated with Zn(CN)₂ (311 mg, 2.66 mmol) and K₂CO₃ (440 mg, 3.19 mmol), and degassed with argon for 15 min. Pd(dppf)Cl₂·DCM (87 mg, 0.106 mmol) was added to the reaction mixture. The reaction was stirred at 120° C. for 2.5 h under microwave radiation (sealed microwave vial). Progress of the reaction was monitored by TLC. TLC mobile phase: 30% EtOAc in pet ether, RF: 0.38, TLC detection: UV. The reaction mixture was diluted with EtOAc (50 mL) and water (50 mL). The organic layer was separated and washed with brine (50 mL), dried over Na₂SO₄ and concentrated under reduced pressure to afford crude product (550 mg, LC/MS 53%) which was purified by normal phase column chromatography (Combi) using a 24 g column and a gradient of 11% EtOAc in pet ether as an eluent to afford tert-butyl (5-cyano-1-(4-(trifluoromethyl)phenyl)-1,2,3,4-tetrahydroquinolin-3-yl)carbamate (Int-X2) as a white solid (180 mg, 40%). (LC/MS; m/z 418.1 [M+H]⁺)

Steps 2-3: These steps were executed in a manner similar (use of appropriate reagents and purification methods known to the person skilled in the art) to Cpd. No. 001. Starting material Int-X² (180 mg, 0.43 mmol, LC/MS 99%) yielded a pale yellow solid (180 mg, LC/MS 82%) which was purified by preparative HPLC method H3. The collected fractions were concentrated under reduced pressure and lyophilised to afford N-(5-cyano-1-(4-(trifluoromethyl)phenyl)-1,2,3,4-tetrahydroquinolin-3-yl)acrylamide (Cpd. No. 078) as an off-white solid (64 mg, 43%). (LC/MS; m/z 372.2 [M+H]⁺)

Step 4: A solution of Int-X2 (400 mg, 0.959 mmol, LC/MS 96%) in EtOH (3.0 mL) was treated with a 50% aq. NaOH solution (3.0 mL) stirred at 80° C. for 1 h under microwave radiation (sealed microwave vial). Progress of the reaction was monitored by TLC. TLC mobile phase: 10% MeOH in DCM, RF: 0.01, TLC detection: UV. The reaction mixture was cooled to RT, concentrated under reduced pressure to afford crude product which was taken in water (4 mL), neutralized with 2 N HCl (aq.) at 0° C. and extracted with 10% MeOH in DCM (2×20 mL). The organic layer was dried over Na₂SO₄ and concentrated under reduced pressure to afford sodium 3-amino-1-(4-(trifluoromethyl) phenyl)-1,2,3,4-tetrahydroquinoline-5-carboxylate as a pale brown gum (400 mg, 86%). (LC/MS; m/z 337.2 [M+H]⁺)

Step 5: A solution of sodium 3-amino-1-(4-(trifluoromethyl)phenyl)-1,2,3,4-tetrahydroquinoline-5-carboxylate (400 mg, 1.11 mmol, LC/MS 74%) in THF (5.0 mL) and water (0.6 mL) was cooled to 0° C., treated with a solution of acryloyl chloride (86 mg, 1.190 mmol) in THF (1.0 mL). The reaction mixture was stirred at 0° C. for 15 min. Progress of the reaction was monitored by TLC. TLC mobile phase: 10% MeOH in DCM, RF: 0.50, TLC detection: UV. The reaction mixture was diluted with water (20 mL) and extracted with EtOAc (2×30 mL). The combined organic layer was washed with sat. brine (20 mL), dried over Na₂SO₄ and concentrated under reduced pressure to afford crude product (250 mg, LC/MS 89%) which was purified by preparative HPLC method H10. The collected fractions were concentrated and lyophilized to afford 3-acrylamido-1-(4-(trifluoromethyl)phenyl)-1,2,3,4-tetrahydroquinoline-5-carboxylic acid (Cpd. No. 079) as an off-white solid (53 mg, 16%). (LC/MS; m/z 391.3 [M+H]⁺)

### Example 36: Synthesis of N-(1-(4-(trifluoromethyl)phenyl)-1,2,3,4-tetrahydro-1,5-naphthyridin-3-yl)acrylamide (Cpd. No. 080)

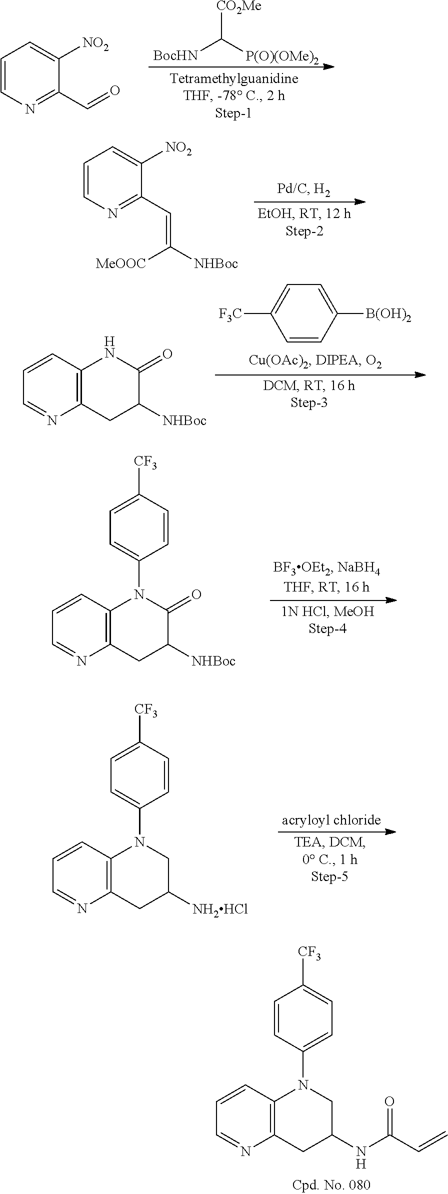

Step 1: A solution of methyl 2-((tert-butoxycarbonyl)amino)-2-(dimethoxyphosphoryl)acetate (4.3 g, 13.9 mmol) in dry THF (50 mL) was cooled to −78° C. under a nitrogen atmosphere, was treated with tetramethylguanidine (1.6 g, 14.5 mmol) and stirred for 10 min. 3—Nitropicolinaldehyde (2 g, 13.2 mmol) in dry THF (20 mL) was added to the reaction mixture dropwise and stirred at −78° C. for 2 h. Progress of the reaction was monitored by TLC. TLC mobile phase: 50% EtOAc in pet ether. RF: 0.5, TLC detection: UV. The reaction mixture was diluted with water (70 mL), extracted with EtOAc (70 mL) and washed with brine (50 mL). The organic layer was dried over anhydrous Na₂SO₄ and concentrated under reduced pressure to afford methyl (E)-2-((tert-butoxycarbonyl)amino)-3-(3-nitropyridin-2-yl)acrylate as a pale yellow gum (4 g, 92%). (LC/MS; m/z 324.0 [M+H]⁺)

Step 2: A solution of methyl (E)-2-((tert-butoxycarbonyl)amino)-3-(3-nitropyridin-2-yl)acrylate (3.9 g, 11.8 mmol) in EtOH (70 mL) was treated with 10% Pd/C (1.2 g) under nitrogen. The reaction mixture was stirred at RT for 24 h under a hydrogen atmosphere (balloon pressure). Progress of the reaction was monitored by TLC. TLC mobile phase: 70% EtOAc in pet ether. RF: 0.1, TLC detection: UV. The reaction mixture was filtered through a celite pad and washed with EtOH (50 mL). The filtrate was concentrated under reduced pressure to afford crude product as a pale yellow gum (3.9 g), which was purified by normal phase chromatography (Grace) using neutral alumina and a gradient of 28% EtOAc in pet ether. The collected fractions were concentrated to afford tert-butyl (2-oxo-1,2,3,4-tetrahydro-1,5-naphthyridin-3-yl)carbamate as a pale yellow solid (1.7 g, 53%). (LC/MS; m/z 264.1 [M+H]⁺)

Step 3: A solution of tert-butyl (2-oxo-1,2,3,4-tetrahydro-1,5-naphthyridin-3-yl)carbamate (1.7 g, 6.5 mmol) in DCM (30 mL) was treated with Cu(OAc)₂ (2.4 g, 13.2 mmol), DIPEA (4.9 g, 37.9 mmol) and (4-(trifluoromethyl)phenyl)boronic acid (1.85 g, 9.7 mmol) and stirred at RT for 16 h under oxygen atmosphere. Progress of the reaction was monitored by TLC. TLC mobile phase: 70% EtOAc in pet ether, RF: 0.3, TLC detection: UV. The reaction mixture was filtered through a celite pad and washed with DCM (50 mL). The filtrate was washed with water (30 mL), then brine (30 mL). The organic layer was dried over Na₂SO₄, filtered and the filtrate was concentrated under reduced pressure to afford crude product as an off-white solid (1.5 g, LC/MS 61%), which was purified by normal phase chromatography using silica (100-200 mesh, 15 g) and a gradient of 45% EtOAc in pet ether. The collected fractions were concentrated to afford tert-butyl (2-oxo-1-(4-(trifluoromethyl)phenyl)-1,2,3,4-tetrahydro-1,5-naphthyridin-3-yl)carbamate as an off-white solid (750 mg, 29%). (LC/MS; m/z 408.0 [M+H]⁺)

Step 4: To a solution of NaBH₄ (45 mg, 1.22 mmol) in THF (5 mL) was added BF₃·OEt₂ (697 mg, 4.91 mmol) at 0° C. and stirred at RT for 15 min. To this solution was added a solution of tert-butyl (2-oxo-1-(4-(trifluoromethyl)phenyl)-1,2,3,4-tetrahydro-1,5-naphthyridin-3-yl)carbamate (250 mg, 0.61 mmol) in THF (5 mL) at 0° C. and stirred at RT for 16 h. Progress of the reaction was monitored by TLC. TLC mobile phase: 5% MeOH in DCM. RF: 0.15, TLC detection: UV. The reaction mixture was quenched with MeOH (20 mL), 1N HCl (20 mL) and concentrated to afford 1-(4-(trifluoromethyl)phenyl)-1,2,3,4-tetrahydro-1,5-naphthyridin-3-amine hydrochloride as an off-white solid (850 mg, LC/MS 89%). (LC/MS; m/z 294.2 [M+H]⁺)

Step 5: This step was executed in a manner similar (use of appropriate reagents and purification methods known to the person skilled in the art) to Cpd. No. 001. Starting material (750 mg, 2.3 mmol, LC/MS 89%) yielded a yellow gum (180 mg, LC/MS 36%), which was purified by preparative HPLC method H3. The obtained fractions were lyophilised to afford N-(1-(4-(trifluoromethyl)phenyl)-1,2,3,4-tetrahydro-1,5-naphthyridin-3-yl)acrylamide (Cpd. No. 080) as a white solid (13 mg). (LC/MS; m/z 348.2 [M+H]⁺)

### Example 37: Synthesis of N-(1-(4-(trifluoromethyl)phenyl)-1,2,3,4-tetrahydro-1,8-naphthyridin-3-yl)acrylamide (Cpd. No. 081)

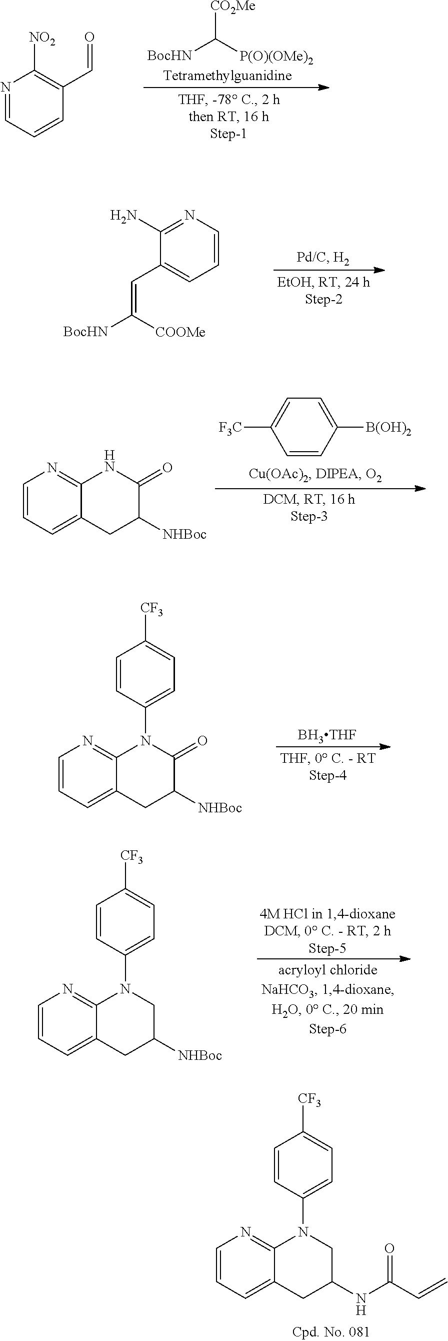

Steps 1-3: These steps were executed in a manner similar (use of appropriate reagents and purification methods known to the person skilled in the art) to steps 1-3 towards Cpd. No. 080.2-Nitronicotinaldehyde (10 g, 66 mmol) yielded crude product (7.8 g, LC/MS 45%) which was purified by normal phase column chromatography (Grace) using a 120 g column and a gradient of 30% EtOAc in pet ether as an eluent to afford tert-butyl (2-oxo-1-(4-(trifluoromethyl)phenyl)-1,2,3,4-tetrahydro-1,8-naphthyridin-3-yl)carbamate as a white solid (3.25 g, 12%). (LC/MS; m/z 408.5 [M+H]⁺)

Step 4: A solution of tert-butyl (2-oxo-1-(4-(trifluoromethyl)phenyl)-1,2,3,4-tetrahydro-1,8-naphthyridin-3-yl)carbamate (1 g, 2.5 mmol, LC/MS 94%) in THF (10 mL) was cooled to 0° C., treated with BH₃·THF (1M in THF, 12.5 mL, 12.5 mmol) under a nitrogen atmosphere and stirred at RT for 8 h. An additional 2 eq. of BH₃·THF (1M in THF, 5.0 mL, 5.0 mmol) was added at 0° C. and stirred at RT for 8 h. Progress of the reaction was monitored by TLC. TLC mobile phase: 30% EtOAc in pet ether, RF: 0.53, TLC detection: UV. The reaction mixture was cooled to 0° C., quenched with MeOH (3 mL) and concentrated to afford crude product which was taken in water (50 mL) and extracted with EtOAc (2×50 mL). The combined organic layer was dried over Na₂SO₄, filtered and concentrated to afford crude product as a white solid (800 mg) which was taken forward in the subsequent reaction without further purification. (LC/MS; m/z 394.3 [M+H]⁺ (14%) and m/z 294.3 [M+H]⁺ (27%))

Steps 5-6: These steps were executed in a manner similar (use of appropriate reagents and purification methods known to the person skilled in the art) to Cpd. No. 001. Starting material (800 mg, 2.03 mmol, LC/MS 14%+27% of de-Boc product) yielded a pale yellow gum (400 mg, LC/MS 59%) which was purified by normal phase column chromatography (Combi) using a 24 g column and a gradient of 30% EtOAc in pet ether as an eluent to afford the product as an off-white solid (250 mg, LC/MS 66%). The product was further purified by preparative HPLC method H3. The collected fractions were concentrated under reduced pressure and lyophilised to afford N-(1-(4-(trifluoromethyl) phenyl)-1,2,3,4-tetrahydro-1,8-naphthyridin-3-yl)acrylamide (Cpd. No. 081) as a white solid (50 mg, 14%). (LC/MS; m/z 348.2 [M+H]⁺)

### Example 38: Synthesis of N-(1-(4-(trifluoromethyl)phenyl)-1,2,3,4-tetrahydro-1,6-naphthyridin-3-yl)acrylamide (Cpd. No. 082)

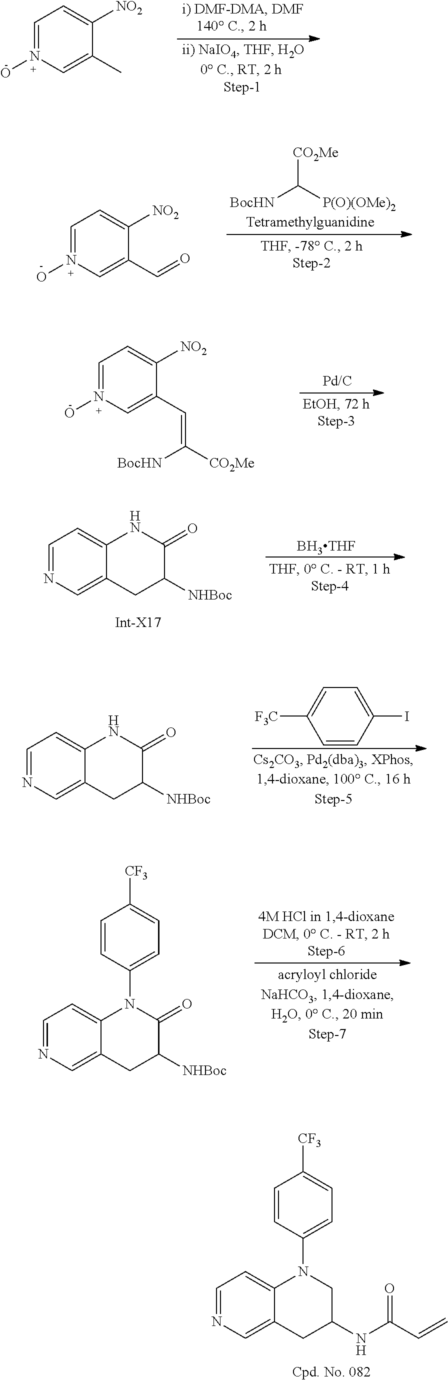

Step 1: A solution of 3-methyl-4-nitropyridine 1-oxide (10.0 g, 64.9 mmol) in DMF (35 mL) was treated with DMF-DMA (12.5 g, 105.1 mmol) under a nitrogen atmosphere at RT and stirred at 140° C. for 2 h. The reaction mixture was cooled to RT and evaporated under reduced pressure to afford the residue which was taken in THF (50 mL) and added to a stirred solution of NaIO₄ (41.6 g, 194.7 mmol) in THF (300 mL) and water (350 mL) at 0° C. The reaction mixture was stirred at RT for 2 h and the progress was monitored by TLC. TLC mobile phase: 5% MeOH in DCM, RF: 0.32, TLC detection: UV. The reaction mixture was diluted with water (500 mL) and extracted with EtOAc (2×150 mL). The organic layer was washed with water (2×200 mL), dried over Na₂SO₄ and concentrated to afford crude product (10.0 g) which was taken forward in the subsequent reaction without further purification.

Steps 2-3: These steps were executed in a manner similar (use of appropriate reagents and purification methods known to the person skilled in the art) to steps 1-2 towards Cpd. No. 080. 3-formyl-4-nitropyridine 1-oxide (10.0 g, 62.5 mmol) yielded crude product (2.8 g) which was purified by normal phase column chromatography using neutral alumina and a gradient of 3% MeOH in DCM as an eluent to afford tert-butyl (2-oxo-1,2,3,4-tetrahydro-1,6-naphthyridin-3-yl)carbamate (Int-X17) as an off-white solid (450 mg, 19%). (LC/MS; m/z 264.2 [M+H]⁺)

Step 4: A solution of (Int-X17) (200 mg, 0.76 mmol) in THF (6 mL) was cooled to 0° C. and treated dropwise with BH₃·THF (1M in THF) (4.56 mL, 4.56 mmol). The reaction mixture was stirred under a nitrogen atmosphere at RT for 1 h and the progress was monitored by TLC. TLC mobile phase: 10% MeOH in DCM, RF: 0.04, TLC detection: UV. The reaction mixture was cooled to RT, quenched with MeOH (3.0 mL), concentrated, diluted with DCM (30.0 mL) and water (30.0 mL). The organic layer was separated and washed with brine (30.0 mL), dried over Na₂SO₄ and concentrated under reduced pressure to afford crude product (170 mg, LC/MS 53%) which was purified by flash column chromatography using neutral alumina and a gradient of 10% MeOH in DCM containing 2% TEA as an eluent to afford tert-butyl (1,2,3,4-tetrahydro-1,6-naphthyridin-3-yl)carbamate as an off-white solid (130 mg, 22%). (LC/MS; m/z 250.1 [M+H]⁺)

Step 5: A solution of tert-butyl (1,2,3,4-tetrahydro-1,6-naphthyridin-3-yl)carbamate (100 mg, 0.40 mmol) in 1,4-dioxane (3.0 mL) (sealed tube) was treated with 1-iodo-4-(trifluoromethyl)benzene (218 mg, 0.80 mmol), Cs₂CO₃ (262 mg, 0.80 mmol) and degassed with argon for 15 min. XPhos (38 mg, 0.08 mmol) and Pd₂(dba)₃ (37 mg, 0.040 mmol) were added to the reaction mixture, stirred at 100° C. for 16 h, and the progress was monitored by TLC. TLC mobile phase: 10% MeOH in DCM, RF: 0.42, TLC detection: UV. The reaction mixture was cooled to RT and diluted with EtOAc (20.0 mL) and water (20.0 mL). The organic layer was washed with water (20.0 mL), dried over Na₂SO₄ and concentrated under reduced pressure to afford crude product (200 mg) which was purified by normal phase column chromatography using a 12 g column and a gradient of 6% MeOH in DCM as an eluent to afford tert-butyl (1-(4-(trifluoromethyl)phenyl)-1,2,3,4-tetrahydro-1,6-naphthyridin-3-yl)carbamate as a pale brown gum (100 mg, 60%). (LC/MS; m/z 394.3 [M+H]⁺)

Steps 6-7: These steps were executed in a manner similar (use of appropriate reagents and purification methods known to the person skilled in the art) to Cpd. No. 001. tert-butyl (1-(4-(trifluoromethyl)phenyl)-1,2,3,4-tetrahydro-1,6-naphthyridin-3-yl)carbamate (100 mg, 0.25 mmol) yielded a pale brown gum (70 mg, LC/MS 34%) which was purified by preparative HPLC method H9. The collected fractions were concentrated under reduced pressure and lyophilised to afford N-(1-(4-(trifluoromethyl)phenyl)-1,2,3,4-tetrahydro-1,6-naphthyridin-3-yl) acrylamide (Cpd. No. 082) as a white solid (6 mg, 8%). (LC/MS; m/z 348.2 [M+H]⁺)

### Example 39: Synthesis of N-(1-(4-(trifluoromethyl)phenyl)-1,2,3,4-tetrahydro-1,7-naphthyridin-3-yl)acrylamide (Cpd. No. 083)

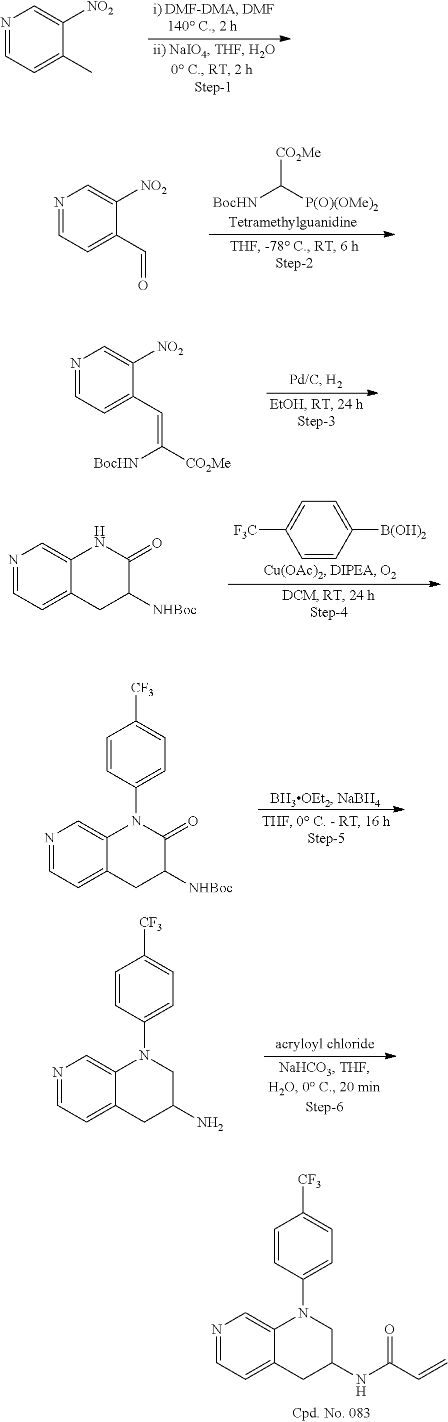

Step 1: This step was executed in a manner similar (use of appropriate reagents and purification methods known to the person skilled in the art) to step 1 towards Cpd. No. 082. 4-methyl-3-nitropyridine (5.0 g, 36.2 mmol) yielded crude product (5.0 g) which was taken forward in the subsequent reaction without further purification.

Steps 2-4: These steps were executed in a manner similar (use of appropriate reagents and purification methods known to the person skilled in the art) to steps 1-3 towards Cpd. No. 080. 3-Nitroisonicotinaldehyde (5 g, 32.9 mmol) yielded crude product (5.1 g, LC/MS 21%) which was purified by normal phase column chromatography (Grace) using silica (100-200 mesh) and a gradient of 45% EtOAc in pet ether to afford tert-butyl (2-oxo-1-(4-(trifluoromethyl)phenyl)-1,2,3,4-tetrahydro-1,7-naphthyridin-3-yl)carbamate as a pale yellow solid (1.25 g, 9%). (LC/MS; m/z 408.5 [M+H]⁺)

Step 5: A solution of tert-butyl (2-oxo-1-(4-(trifluoromethyl)phenyl)-1,2,3,4-tetrahydro-1,7-naphthyridin-3-yl)carbamate (300 mg, 0.74 mmol, LC/MS 94%) in THF (5 mL) was cooled to 0° C., treated with BF₃·OEt₂ (523 mg, 3.68 mmol) and portionwise with NaBH₄ (168 mg, 4.42 mmol) under a nitrogen atmosphere. The reaction mixture was stirred at RT for 16 h. Progress of the reaction was monitored by TLC. TLC mobile phase: 10% MeOH in DCM, RF: 0.1, TLC detection: UV. The reaction mixture was cooled to 0° C. and quenched with MeOH (5 mL) at 0° C. and concentrated under reduced pressure. The residue which was taken in 10% MeOH in DCM (20 mL), washed with brine (20 mL), dried over Na₂SO₄ and concentrated to afford 1-(4-(trifluoromethyl)phenyl)-1,2,3,4-tetrahydro-1,7-naphthyridin-3-amine as a pale brown gum (180 mg, 51%) which was taken forward in the subsequent reaction step without further purification. (LC/MS; m/z 294.2 [M+H]⁺)

Step 6: This step was executed in a manner similar (use of appropriate reagents and purification methods known to the person skilled in the art) to Cpd. No. 001. 1-(4-(trifluoromethyl)phenyl)-1,2,3,4-tetrahydro-1,7-naphthyridin-3-amine (180 mg, LC/MS 58%) yielded crude product (175 mg, LC/MS 36%) which was purified by normal phase column chromatography (Grace) using a 12 g column and 5% of MeOH in DCM as an eluent to afford crude product (150 mg, LC/MS 42%) which was further purified by preparative HPLC method H3. The collected fractions were concentrated under reduced pressure and lyophilised to afford N-(1-(4-(trifluoromethyl)phenyl)-1,2,3,4-tetrahydro-1,7-naphthyridin-3-yl)acrylamide (Cpd. No. 083) as an off-white solid (24 mg, 19%). (LC/MS; m/z 348.2 [M+H]⁺)

### Example 40: Synthesis of N-((1-(4-(trifluoromethyl)phenyl)-1,2,3,4-tetrahydroquinolin-3-yl)methyl)acrylamide (Cpd. No. 084)

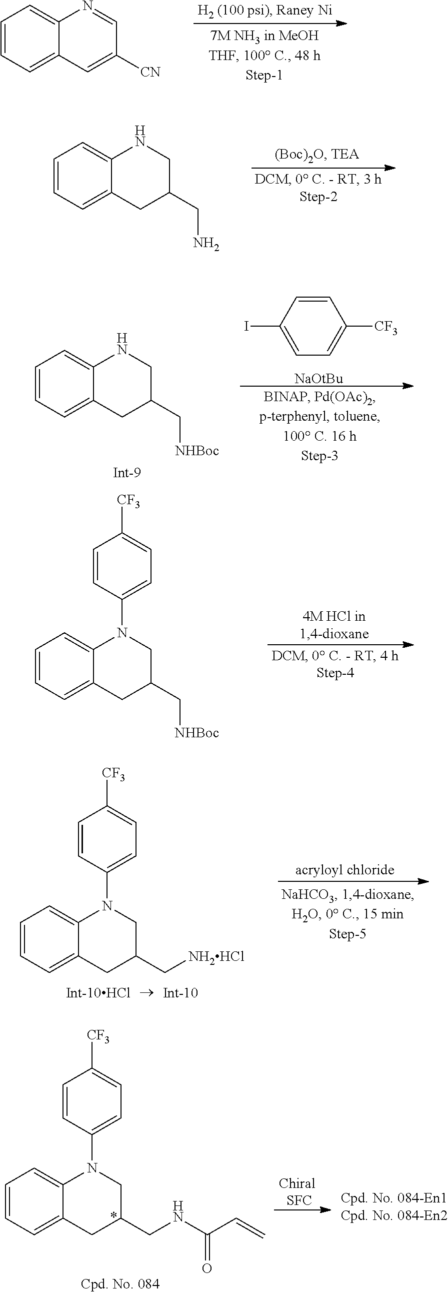

Step 1: A solution of quinoline-3-carbonitrile (2.0 g, 13 mmol) in THF (25 mL) was treated with Raney Ni (2.0 g, 100% w/w) and 7M NH₃ in MeOH (4 mL) at RT. The reaction mixture was stirred at 100° C. under a hydrogen atmosphere (100 psi) for 48 h in 100 mL in a steal bomb. Progress of the reaction was monitored by TLC. TLC mobile phase: 50% EtOAc in pet ether, RF: 0.5, TLC detection: UV. The reaction mixture was filtered through a celite pad, the filtrate was concentrated under reduced pressure to afford (1,2,3,4-tetrahydroquinolin-3-yl)methanamine (2.0 g, 73%) as a pale yellow gum which was taken forward in the subsequent reaction without further purification. (LC/MS; m/z 163.1 [M+H]⁺)

Step 2: A solution of (1,2,3,4-tetrahydroquinolin-3-yl)methanamine (2.00 g, 12.3 mmol) in DCM (30 mL) was treated with (Boc)₂O (2.68 g, 12.3 mmol) and TEA (1.87 g, 18.45 mmol) at 0° C. and stirred for 3 h at RT. Progress of the reaction was monitored by TLC. TLC mobile phase: 10% EtOAc in pet ether, RF: 0.6, TLC detection: UV. The reaction was partitioned between DCM (30 mL) and water (40 mL). The organic layer was separated, washed with brine (20 mL), dried over Na₂SO₄ and concentrated under reduced pressure to afford crude product (2.3 g, LC/MS 34%) which was purified by normal phase chromatography using silica gel (100-200 mesh) and a gradient of 4% EtOAc in pet ether to afford tert-butyl ((1,2,3,4-tetrahydroquinolin-3-yl)methyl)carbamate (Int-9) as an off-white solid (1.5 g, 46%). (LC/MS; m/z 263.3 [M+H]⁺)

Step 3: A solution of tert-butyl ((1,2,3,4-tetrahydroquinolin-3-yl)methyl)carbamate (Int-9) (500 mg, 1.90 mmol) in toluene (20 mL) was treated with 1-iodo-4-(trifluoromethyl)benzene (1.03 g, 3.81 mmol), BINAP (118 mg, 0.19 mmol), p-terphenyl (100 mg, 0.43 mmol) and NaOtBu (256 mg, 2.66 mmol) degassed with argon for 15 min at RT. Pd(OAc)₂ (64 mg, 0.09 mmol) was added to the reaction mixture, degassed for 5 min again and stirred at 100° C. for 16 h under an argon atmosphere. Progress of the reaction was monitored by TLC. TLC mobile phase: 20% EtOAc in pet ether, RF: 0.5, TLC detection: UV. The reaction mixture was cooled to RT, filtered through a celite pad and washed with EtOAc (10 mL). The filtrate was partitioned between EtOAc (80 mL) and water (50 mL). The organic layer was separated, washed with brine (40 mL), dried over Na₂SO₄ and concentrated under reduced pressure to afford crude product (650 mg, LC/MS 20%) which was purified by normal phase chromatography using silica (100-200 mesh) and a gradient of 10% of EtOAc in pet ether to afford tert-butyl ((1-(4-(trifluoromethyl)phenyl)-1,2,3,4-tetrahydroquinolin-3-yl)methyl)carbamate (270 mg, 34%) as an orange gum. (LC/MS; m/z 407.8 [M+H]⁺)

Step 4: A solution of tert-butyl ((1-(4-(trifluoromethyl)phenyl)-1,2,3,4-tetrahydroquinolin-3-yl)methyl)carbamate (270 mg, 0.66 mmol) in DCM (7 mL) was treated with 4 M HCl solution in 1,4-dioxane (1.5 mL) at 0° C. and stirred at RT for 4 h. Progress of the reaction monitored by TLC. TLC mobile phase: 10% MeOH in DCM, RF: 0.54, TLC detection: UV. The reaction mixture was concentrated under reduced pressure to afford (1-(4-(trifluoromethyl)phenyl)-1,2,3,4-tetrahydroquinolin-3-yl)methanamine hydrochloride (Int-10·HCl) as a pale yellow gum (250 mg crude). The crude mixture was dissolved in DCM and washed with a sat. aq. NaHCO₃ solution (3 20×20 mL) and brine solution (20 mL). The organic layer was dried over Na₂SO₄ and concentrated under reduced pressure. The product was purified by preparative HPLC method H4. The collected fractions were lyophilised and afforded (1-(4-(trifluoromethyl)phenyl)-1,2,3,4-tetrahydroquinolin-3-yl)methanamine (Int-10; Cpd. No. 170) as an off-white gum (112 mg, 54%). (LC/MS; m/z 307.5 [M+H]⁺)

Step 5: A solution of (1-(4-(trifluoromethyl)phenyl)-1,2,3,4-tetrahydroquinolin-3-yl)methanamine hydrochloride (250 mg, 0.72 mmol) in 1,4-dioxane (8 mL) was treated with aq. NaHCO₃ (122 mg, 1.458 mmol in 0.4 mL water) followed by a solution of acryloyl chloride (78 mg, 0.875 mmol) in 1,4-dioxane (2 mL) at 0° C. The reaction mixture was stirred for 15 min at 0° C. Progress of the reaction was monitored by TLC. TLC mobile phase: 50% EtOAc in pet ether, RF: 0.61, TLC detection: UV. The reaction mixture was partitioned between EtOAc (10 mL) and water (10 mL). The organic layer was separated, washed with brine (10 mL), dried over Na₂SO₄ and concentrated under reduced pressure to afford crude product (330 mg) which was purified by preparative HPLC method H3. The collected fractions were lyophilised to afford N-((1-(4-(trifluoromethyl)phenyl)-1,2,3,4-tetrahydroquinolin-3-yl)methyl)acrylamide as an off-white solid (Cpd. No. 084) (65 mg, 25%). (LC/MS; m/z 361.3 [M+H]⁺) Chiral SFC purification: 130 mg of Cpd. No. 084 was further purified by preparative SFC method K1 to afford Cpd. No. 084-En1 (30 mg) and Cpd. No. 084-En2 (35 mg), both as an off-white solid. (LC/MS; m/z 361.3 [M+H]⁺) The chiral purity of both enantiomers was assessed by analytic SFC method S3: Cpd. No. 084-En1, 98.4% ee; Cpd. No. 084-En2, 94.8% ee.

From Int-9, the following compounds were prepared in a manner similar to Cpd. No. 084 by using appropriate reagents and purification methods known to the person skilled in the art: Cpd. No. 085, Cpd. No. 086, Cpd. No. 087, Cpd. No. 088, Cpd. No. 089, Cpd. No. 090, Cpd. No. 091, Cpd. No. 092, Cpd. No. 093, Cpd. No. 094, Cpd. No. 095, Cpd. No. 096, Cpd. No. 097, Cpd. No. 098, and Cpd. No. 099.

### Example 41: Synthesis of 4-(3-(acrylamidomethyl)-3,4-dihydroquinolin-1(2H)-yl)—N-methylbenzamide (Cpd. No. 100)

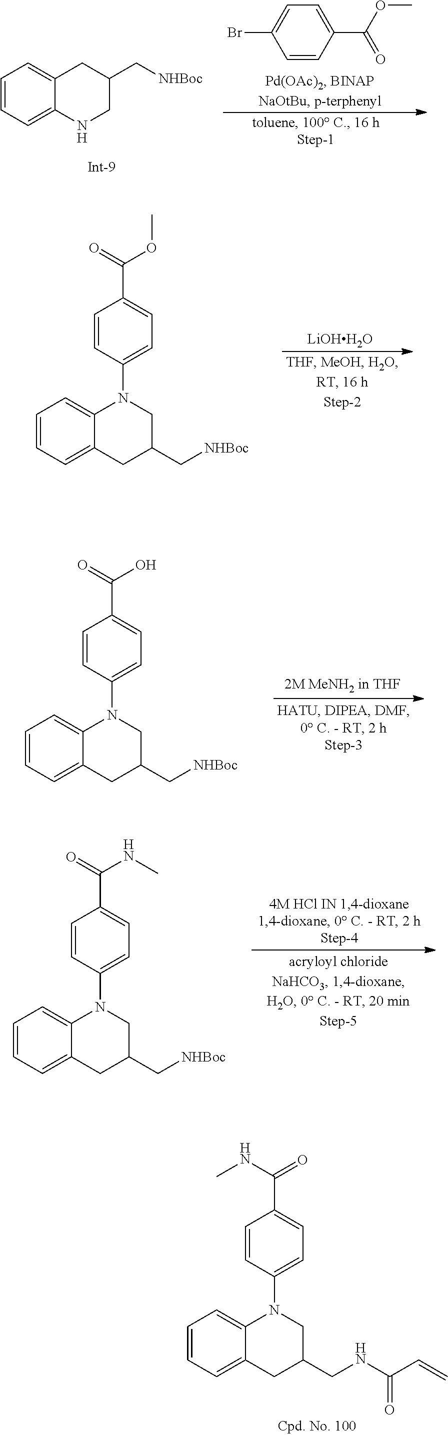

Step 1: This step was executed with Int-9 (400 mg, 1.52 mmol, LC/MS 92%) in a manner similar (use of appropriate reagents and purification methods known to the person skilled in the art) to Cpd. No. 084, yielding crude product as a brown gum which was purified by normal phase column chromatography (Grace) using 24 g column (silica gel) and a gradient of 15% EtOAc in pet ether as an eluent to afford methyl 4-(3-(((tert-butoxycarbonyl)amino)methyl)-3,4-dihydroquinolin-1(2H)-yl)benzoate as a pale yellow gum (300 mg, 52%). (LC/MS; m/z 397.1 [M+H]⁺)

Step 2: A solution of methyl 4-(3-(((tert-butoxycarbonyl)amino)methyl)-3,4-dihydroquinolin-1(2H)-yl)benzoate (300 mg, 0.757 mmol, LC/MS 96%) in THF (1 mL), MeOH (1 mL) and water (1 mL) was treated with LiOH·H₂O (127 mg, 3.03 mmol) at RT and stirred for 16 h. The reaction progress was monitored by TLC. TLC mobile phase: 70% EtOAc in pet ether, RF: 0.30, TLC detection: UV. The reaction mixture was acidified with 2N HCl (2 mL), diluted with water (5 mL) and extracted with EtOAc (2×25 mL). The combined organic layer was dried over Na₂SO₄, filtered and concentrated under reduced pressure to afford 4-(3-(((tert-butoxycarbonyl) amino)methyl)-3,4-dihydroquinolin-1(2H)-yl)benzoic acid as a pale yellow gum (280 mg, 91%). (LC/MS; m/z 383.1 [M+H]⁺)

Step 3: A solution of 4-(3-(((tert-butoxycarbonyl)amino)methyl)-3,4-dihydroquinolin-1(2H)-yl)benzoic acid (280 mg, 0.733 mmol, LC/MS 91%) in DMF (3 mL) was cooled to 0° C., treated with HATU (557 mg, 1.46 mmol), DIPEA (236 mg, 1.83 mmol) under a nitrogen atmosphere and stirred for 5 min. The solution was treated with a solution of methylamine (2M in THF, 1.4 mL, 2.92 mmol) and stirred at RT for 2 h. The reaction progress was monitored by TLC. TLC mobile phase: 70% EtOAc in pet ether, RF: 0.51, TLC detection: UV. The reaction mixture was diluted with water (20 mL) and extracted with EtOAc (2×50 mL). The combined organic layer was dried over Na₂SO₄, filtered and concentrated under reduced pressure to afford crude product as a yellow gum (310 mg, LC/MS 81%) which was purified by normal phase column chromatography (Grace) using 12 g column (silica gel) and a gradient of 25% EtOAc in pet ether as an eluent to afford tert-butyl ((1-(4-(methylcarbamoyl)phenyl)-1,2,3,4-tetrahydroquinolin-3-yl)methyl)carbamate as a yellow gum (250 mg, 84%). (LC/MS; m/z 396.0 [M+H]⁺)

Steps 4-5: These steps were executed in a manner similar (use of appropriate reagents and purification methods known to the person skilled in the art) to Cpd. No. 084. Starting material (250 mg, 0.632 mmol, LC/MS 88%) yielding a pale yellow gum (240 mg, LC/MS 74%), which was purified by preparative HPLC method H3. The collected fractions were concentrated and lyophilised under vacuum to afford 4-(3-(acrylamidomethyl)-3,4-dihydroquinolin-1(2H)-yl)—N-methylbenzamide (Cpd. No. 100) as a white solid (58 mg, 26%). (LC/MS; m/z 350.3 [M+H]⁺)

### Example 42: Synthesis of N-((1-(3,4-difluorophenyl)-1,2,3,4-tetrahydroquinolin-3-yl)methyl)acrylamide (Cpd. No. 101)

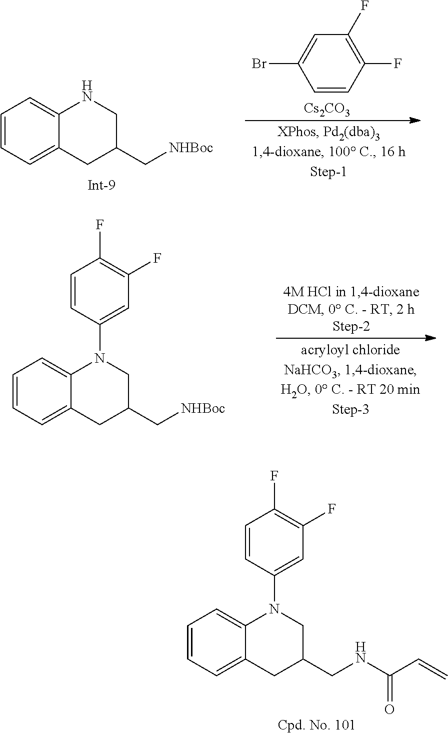

Step 1: A solution of tert-butyl ((1,2,3,4-tetrahydroquinolin-3-yl)methyl)carbamate (Int-9) (400 mg, 1.52 mmol, LC/MS 92%) in 1,4-dioxane (10 mL) (sealed tube) was treated with 4-bromo-1,2-difluorobenzene (589 mg, 3.05 mmol) and Cs₂CO₃ (995 mg, 3.05 mmol) at RT. The reaction mixture was degassed with argon for 5 min, treated with XPhos (143 mg, 0.305 mmol), Pd₂(dba)₃ (139 mg, 0.15 mmol) and stirred at 100° C. for 16 h. Progress of the reaction was monitored by TLC. TLC mobile phase: 20% EtOAc in pet ether, RF: 0.52, TLC detection: UV. The reaction mixture was cooled to RT, diluted with water (50 mL) and extracted with EtOAc (2×50 mL). The combined organic layer was dried over Na₂SO₄ and concentrated under reduced pressure to afford crude product as a yellow gum (500 mg, LC/MS 46%) which was purified by normal phase column chromatography (Grace) using 12 g column and a gradient of 15% EtOAc in petether as an eluent to afford tert-butyl ((1-(3,4-difluorophenyl)-1,2,3,4-tetrahydroquinolin-3-yl)methyl)carbamate as an off-white solid (350 mg, 50%). (LC/MS; m/z 375.1 [M+H]⁺)

Steps 2-3: These steps were executed in a manner similar (use of appropriate reagents and purification methods known to the person skilled in the art) to Cpd. No. 084. Starting material (350 mg, 0.935 mmol, LC/MS 76%) yielded a pale yellow gum (350 mg, LC/MS 76%), which was purified by preparative HPLC method H5. The collected fractions were concentrated and lyophilised under vacuum to afford N-((1-(3,4-difluorophenyl)-1,2,3,4-tetrahydroquinolin-3-yl)methyl)acrylamide (Cpd. No. 101) as an off-white solid (124 mg, 40%). (LC/MS; m/z 329.3 [M+H]⁺)

From Int-9, the following compounds were prepared in a manner similar (use of appropriate reagents and purification methods known to the person skilled in the art) to Cpd. No. 101 by using appropriate reagents and purification methods known to the person skilled in the art: Cpd. No. 102, Cpd. No. 103, Cpd. No. 104, Cpd. No. 105, Cpd. No. 106, Cpd. No. 107, Cpd. No. 108, Cpd. No. 109, Cpd. No. 110, Cpd. No. 111, Cpd. No. 112, Cpd. No. 113, Cpd. No. 114, Cpd. No. 115, Cpd. No. 116, Cpd. No. 117, Cpd. No. 147, Cpd. No. 148, Cpd. No. 151, Cpd. No. 149, Cpd. No. 152, and Cpd. No. 159.

### Example 43: Synthesis of N-((1-cyclohexyl-1,2,3,4-tetrahydroquinolin-3-yl)methyl)acrylamide (Cpd. No. 118)

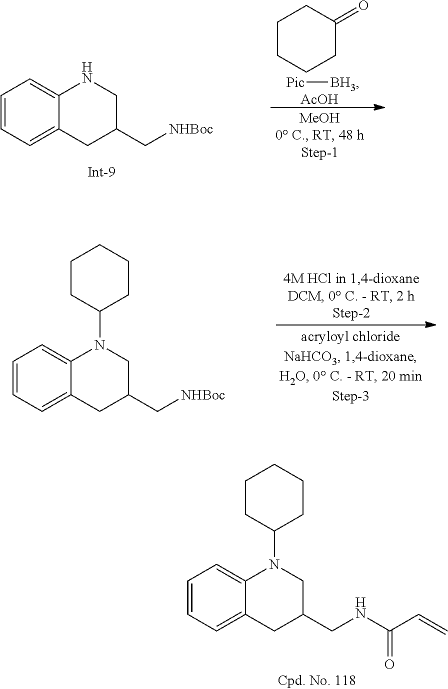

Step 1: A solution of tert-butyl ((1,2,3,4-tetrahydroquinolin-3-yl)methyl)carbamate (Int-9) (600 mg, 2.29 mmol) and cyclohexanone (897 mg, 9.16 mmol) in MeOH (6 mL) was cooled to 0° C., treated with AcOH (0.002 mL) and 2-picoline borane (943 mg, 9.16 mmol) under a nitrogen atmosphere. The reaction mixture was stirred at RT for 48 h. Progress of the reaction was monitored by TLC. TLC mobile phase: 20% EtOAc in pet ether, RF: 0.42, TLC detection: UV. The reaction mixture was diluted with water (30 mL) and the product was extracted with EtOAc (2×30 mL). The organic layer was washed with brine (30 mL), dried over Na₂SO₄ and evaporated under reduced pressure to afford crude product (620 mg, LC/MS 38%) which was purified by normal phase chromatography (GRACE) using 12 g column and a gradient of 5% EtOAc in pet ether as an eluent to afford tert-butyl ((1-cyclohexyl-1,2,3,4-tetrahydroquinolin-3-yl)methyl)carbamate as a pale yellow oil (150 mg, 19%). (LC/MS; m/z 345.4 [M+H]⁺)

Steps 2-3: These steps were executed in a manner similar (use of appropriate reagents and purification methods known to the person skilled in the art) to Cpd. No. 084. Starting material (115 mg, 0.27 mmol) yielded a crude product (140 mg, LC/MS 42%), which was purified by preparative HPLC method H1. The collected fractions were concentrated and lyophilised under vacuum to afford N-((1-cyclohexyl-1,2,3,4-tetrahydroquinolin-3-yl)methyl)acrylamide (Cpd. No. 118) as an off-white solid (11 mg, 11%). (LC/MS; m/z 299.2 [M+H]⁺)

From Int-9, the following compounds were prepared in a manner similar (use of appropriate reagents and purification methods known to the person skilled in the art) to Cpd. No. 118 by using appropriate reagents and purification methods known to the person skilled in the art: Cpd. No. 143, Cpd. No. 144, and Cpd. No. 145.

### Example 44: Synthesis of N-((1-(4-(trifluoromethyl)phenyl)-1,2,3,4-tetrahydroquinolin-3-yl)methyl)propiolamide (Cpd. No. 119)

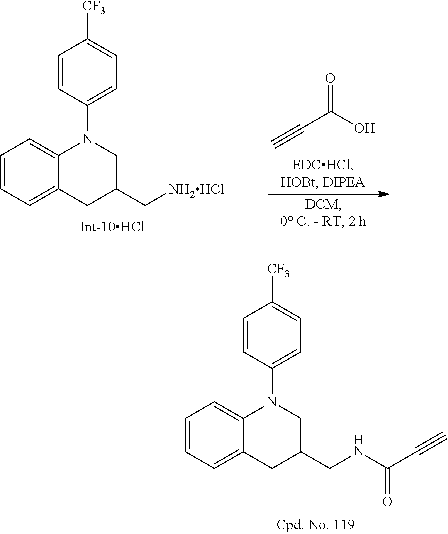

A solution of propiolic acid (102 mg, 1.46 mmol) in DCM (5 mL) was treated with EDC·HCl (280 mg, 1.46 mmol), HOBt (197 mg, 1.46 mmol) and DIPEA (226 mg, 1.75 mmol) at 0° C. and stirred for 10 min. To the solution was added (1-(4-(trifluoromethyl)phenyl)-1,2,3,4-tetrahydroquinolin-3-yl)methanamine hydrochloride (Int-10·HCl) (200 mg, 0.58 mmol) at 0° C. and the reaction mixture was stirred under a nitrogen atmosphere at RT for 2 h. The reaction progress was monitored by TLC. TLC mobile phase: 50% EtOAc in pet ether, RF: 0.68, TLC detection: UV. The reaction mixture was diluted with DCM (20 mL) and water (20 mL). The organic layer was separated and washed with water (20 mL), dried over Na₂SO₄ and concentrated to afford crude product as a brown gum (300 mg, LC/MS 27%) which was purified by normal phase column chromatography (Grace) using 12 g silica gel column and a gradient of 26% EtOAc in pet ether as an eluent to afford the product as a pale brown gum (100 mg, LC/MS 59%). It was further purified by preparative HPLC method H5. The collected fractions were concentrated and lyophilised to afford N-((1-(4-(trifluoromethyl)phenyl)-1,2,3,4-tetrahydroquinolin-3-yl)methyl)propiolamide as an off-white solid (Cpd. No. 119) (18 mg, 9%). (LC/MS; 359.2m/z [M+H]⁺)

Compound Cpd. No. 120 was prepared in a manner similar to Cpd. No. 119 by using appropriate reagents and purification methods known to the person skilled in the art.

### Examples 45-46: Synthesis of N-((1-(4-(trifluoromethyl)phenyl)-1,2,3,4-tetrahydroquinolin-3-yl)methyl)methanesulfonamide (Cpd. No. 121) and N-((1-(4-(trifluoromethyl)phenyl)-1,2,3,4-tetrahydroquinolin-3-yl)methyl)ethenesulfonamide (Cpd. No. 122)

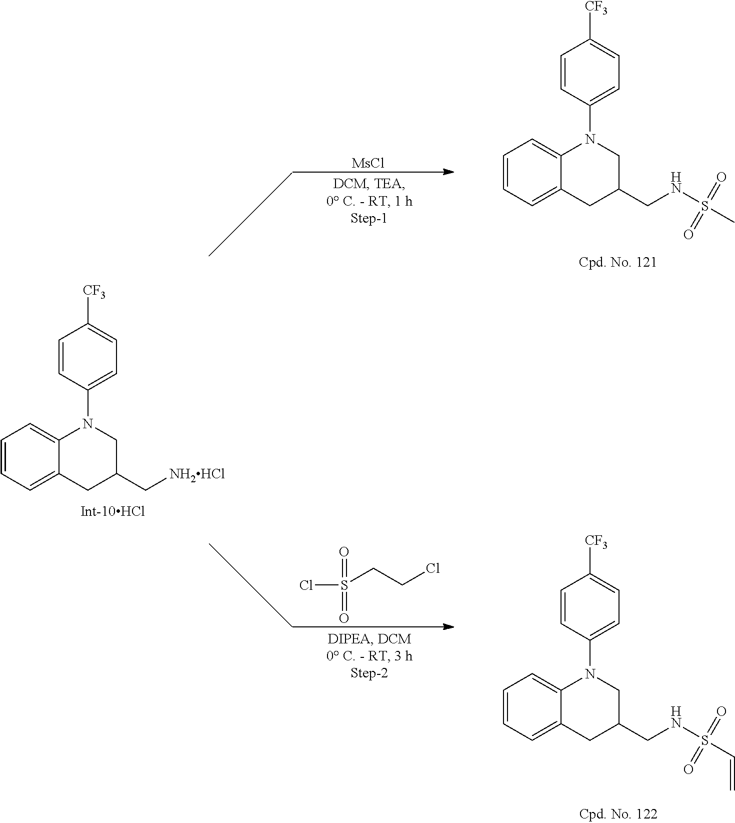

Step 1: A solution of (1-(4-(trifluoromethyl)phenyl)-1,2,3,4-tetrahydroquinolin-3-yl)methanamine hydrochloride (Int-10·HCl) (100 mg, 0.29 mmol) in DCM (2 mL) was cooled to 0° C., treated with TEA (73.7 mg, 0.1 mL, 0.73 mmol) and methanesulfonyl chloride (33.2 mg, 0.01 mL, 0.29 mmol) under an argon atmosphere. The reaction mixture was stirred at RT for 1 h. Progress of the reaction mixture was monitored by TLC. TLC mobile phase: 5% MeOH in DCM, RF: 0.3, TLC detection: UV. The reaction mixture was diluted with water (20 mL) and the product was extracted with DCM (2×20 mL). The organic layer was separated, dried over Na₂SO₄ and evaporated under reduced pressure to afford crude product (160 mg, LC/MS 78.94%) as a brown gum which was purified by preparative HPLC method H3. The collected fractions were concentrated under reduced pressure and lyophilised to afford N-((1-(4-(trifluoromethyl)phenyl)-1,2,3,4-tetrahydroquinolin-3-yl)methyl)methane sulfonamide (Cpd. No. 121) as an off-white solid (54 mg, 48%). (LC/MS; m/z 385.2 [M+H]⁺)

Step 2: A solution of (1-(4-(trifluoromethyl)phenyl)-1,2,3,4-tetrahydroquinolin-3-yl)methanamine hydrochloride (Int-10·HCl) (80 mg, 0.23 mmol), DIPEA (60 mg, 0.46 mmol) in DCM (5 mL) was treated with 2-chloroethane-1-sulfonyl chloride (38 mg, 0.23 mmol) under a nitrogen atmosphere at 0° C. The reaction mixture was stirred at RT for 3 h. The reaction progress was monitored by TLC. TLC mobile phase: 30% EtOAc in pet ether, RF: 0.3, TLC detection: UV. The reaction mixture was diluted with DCM (20 mL) and water (10 mL). The organic layer was separated, dried over Na₂SO₄, filtered and concentrated under reduced pressure to afford crude product as a pale yellow solid (100 mg, LC-MS 41%) which was purified by preparative HPLC method H2. The collected fractions were concentrated under reduced pressure and lyophilised to afford N-((1-(4-(trifluoromethyl)phenyl)-1,2,3,4-tetrahydroquinolin-3-yl)methyl)ethenesulfonamide (Cpd. No. 122) as a pale yellow gum (13 mg, 15%). (LC/MS; m/z 397.2 [M+H]⁺)

### Examples 47-49: Synthesis of 2-((1-(4-(trifluoromethyl)phenyl)-1,2,3,4-tetrahydroquinolin-3-yl)methyl)-1,2-thiazetidine-1,1-dioxide (Cpd. No. 123), N-methyl-2-(((1-(4-(trifluoromethyl)phenyl)-1,2,3,4-tetrahydroquinolin-3-yl)methyl)amino)ethane-1-sulfonamide (Cpd. No. 124), and 2-(((1-(4-(trifluoromethyl)phenyl)-1,2,3,4-tetrahydroquinolin-3-yl)methyl)amino)ethane-1-sulfonic acid (Cpd. No. 183)

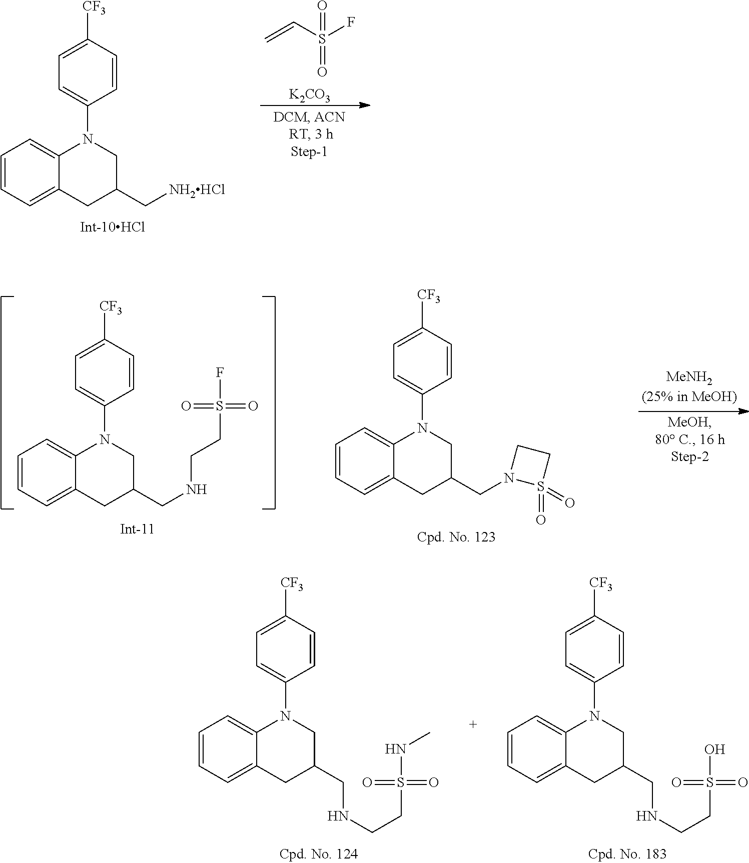

Step 1: A solution of (1-(4-(trifluoromethyl)phenyl)-1,2,3,4-tetrahydroquinolin-3-yl)methanamine hydrochloride (Int-10·HCl) (120 mg, 0.35 mmol) in DCM (5 mL) was treated with K₂CO₃ (96 mg, 0.70 mmol) and ethenesulfonyl fluoride (38 mg, 0.35 mmol) at RT under a nitrogen atmosphere. The reaction mixture was stirred for 3 h at RT. The reaction progress was monitored by TLC. TLC mobile phase: 30% EtOAc in pet ether, RF: 0.34, TLC detection: UV. The reaction mixture was diluted with DCM (10 mL) and washed with water (10 mL). The organic layer was separated, dried over Na₂SO₄, filtered and concentrated under reduced pressure to afford crude product as an off-white gum (135 mg, LC/MS 74% Int-11; m/z 417.2 [M+H]⁺). The crude product was purified by normal phase chromatography using a 12 g reveleris column (Grace) and a gradient of 40% EtOAc in pet ether as an eluent to afford Cpd. No. 123 (64 mg, 37%). During column purification Int-11 is cyclized into Cpd. No. 123. 20 mg of Cpd. No. 123 was further purified by preparative HPLC method H9. The collected fractions were concentrated under reduced pressure and lyophilised to afford 2-((1-(4-(trifluoromethyl)phenyl)-1,2,3,4-tetrahydroquinolin-3-yl)methyl)-1,2-thiazetidine-1,1-dioxide (Cpd. No. 123) as an off-white solid (11 mg, 10%). (LC/MS; m/z 397.3 [M+H]⁺)

Step 2: A solution of 2-(((1-(4-(trifluoromethyl)phenyl)-1,2,3,4-tetrahydroquinolin-3-yl)methyl)amino)ethane-1-sulfonyl fluoride (Int-11) (135 mg, crude, LC/MS 74%) in MeOH (2 mL) was treated with methylamine (25% in MeOH) (2 mL) at RT. The reaction mixture was stirred for 2 h at 80° C. (sealed tube). Progress of the reaction was monitored by TLC. TLC mobile phase: 10% MeOH in DCM, RF: 0.44, TLC detection: UV. The reaction mixture was concentrated under reduced pressure to afford crude product (150 mg, LC/MS 47%). The crude product was purified by normal phase chromatography (Grace) using a 12 g reveleris column and a gradient of 2% MeOH in DCM as an eluent to afford N-methyl-2-(((1-(4-(trifluoromethyl)phenyl)-1,2,3,4-tetrahydroquinolin-3-yl)methyl)amino)ethane-1-sulfonamide (Cpd. No. 124) (20 mg, LC/MS 90%) (LC/MS; m/z 428.2 [M+H]⁺) and 2-(((1-(4-(trifluoromethyl)phenyl)-1,2,3,4-tetrahydroquinolin-3-yl)methyl)amino)ethane-1-sulfonic acid (Cpd. No. 183) (31 mg, 30%, LC/MS 97%) (LC/MS; m/z 415.2 [M+H]⁺). Cpd. No. 124 was further purified by preparative HPLC method H9. The collected fractions were concentrated under reduced pressure and lyophilised to afford an off-white solid (8 mg, 7%).

### Examples 50-52: Synthesis of 2-cyano-N-((1-(4-(trifluoromethyl)phenyl)-1,2,3,4-tetrahydroquinolin-3-yl)methyl)acetamide (Cpd. No. 125), 3-(((1-(4-(trifluoromethyl)phenyl)-1,2,3,4-tetrahydroquinolin-3-yl)methyl)amino)propanenitrile (Cpd. No. 126), and 2-(((1-(4-(trifluoromethyl)phenyl)-1,2,3,4-tetrahydroquinolin-3-yl)methyl)amino)acetonitrile (Cpd. No. 127)

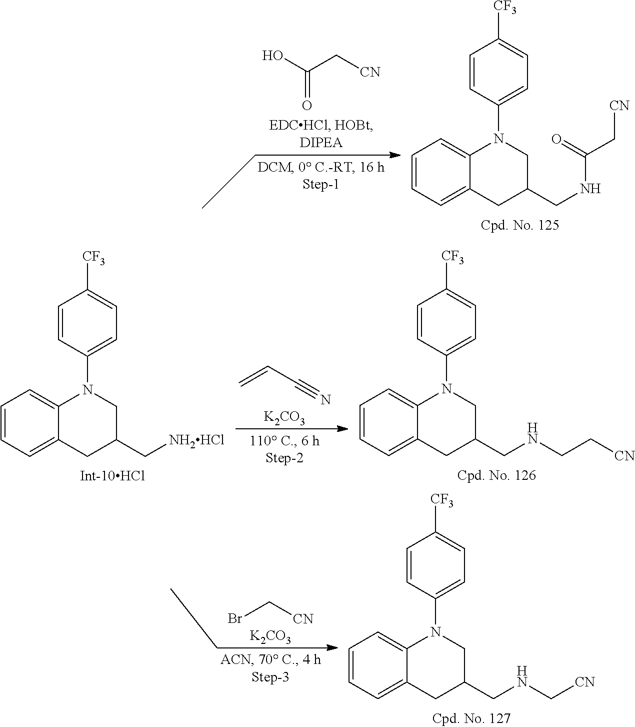

Step 1: A solution of 2-cyanoacetic acid (74.5 mg, 0.87 mmol) in DCM (2 mL) was treated with EDC·HCl (168 mg, 0.87 mmol), HOBt (118 mg, 0.87 mmol) and DIPEA (135.7 mg, 1.05 mmol) at 0° C. and stirred for 15 min at 0° C. (1-(4-(trifluoromethyl)phenyl)-1,2,3,4-tetrahydroquinolin-3-yl)methanamine hydrochloride (Int-10·HCl) (120 mg, 0.35 mmol, LC/MS 98%) was added to the reaction mixture and stirred under a nitrogen atmosphere at RT for 16 h. Progress of the reaction was monitored by TLC. TLC mobile phase: 5% MeOH in DCM, RF: 0.69, TLC detection: UV. The reaction mixture was diluted with water (20 mL) and the product was extracted with DCM (2×20 mL). The organic layer was dried over Na₂SO₄ and concentrated to afford crude product as a pale brown gum (100 mg, LC/MS 85%) which was purified by preparative HPLC method H2. The collected fractions were concentrated and lyophilised to afford 2-cyano-N-((1-(4-(trifluoromethyl)phenyl)-1,2,3,4-tetrahydroquinolin-3-yl)methyl)-acetamide (Cpd. No. 125) as an off-white solid (18 mg, 14%). (LC/MS; m/z 374.2 [M+H]⁺)

Step 2: To (1-(4-(trifluoromethyl)phenyl)-1,2,3,4-tetrahydroquinolin-3-yl)methanamine hydrochloride (Int-10·HCl) (130 mg, 0.380 mmol, LC/MS 90%) (sealed tube) was added acrylonitrile (4 mL) and K₂CO₃ (157 mg, 1.140 mmol) at RT and the reaction mixture was stirred at 110° C. for 6 h. Progress of the reaction TLC mobile phase: 50% EtOAc in pet ether, RF: 0.37, TLC detection: UV. The reaction mixture was diluted with water (20 mL) and the product was extracted with EtOAc (2×20 mL). The organic layer was dried over Na₂SO₄ and concentrated to afford crude product as a pale brown gum (200 mg, LC/MS 56%) which was purified by preparative HPLC method H9. The collected fractions were concentrated and lyophilised to afford 3-(((1-(4-(trifluoromethyl)phenyl)-1,2,3,4-tetrahydroquinolin-3-yl)methyl)amino)propanenitrile (Cpd. No. 126) as an off-white solid (26 mg, 21%). (LC/MS; m/z 360.2 [M+H]⁺)

Step 3: A solution of (1-(4-(trifluoromethyl)phenyl)-1,2,3,4-tetrahydroquinolin-3-yl)methanamine hydrochloride (Int-10·HCl) (150 mg, 0.43 mmol, LC/MS 92%) in ACN (2 mL) (sealed tube) was treated with K₂CO₃ (181.5 mg, 1.31 mmol) and 2-bromoacetonitrile (63 mg, 0.52 mmol) at RT and stirred at 70° C. for 4 h. Progress of the reaction was monitored by TLC. TLC mobile phase: 5% MeOH in DCM, RF: 0.69, TLC detection: UV. The reaction mixture was cooled to RT and diluted with water (30 mL) and extracted with EtOAc (2×30 mL). The organic layer was dried over Na₂SO₄ and concentrated to afford crude product as a pale yellow gum (130 mg, LC/MS 87%) which was purified by preparative HPLC method H4. The collected fractions were concentrated and lyophilised to afford 2-(((1-(4-(trifluoromethyl)phenyl)-1,2,3,4-tetrahydroquinolin-3-yl)methyl)amino)acetonitrile (Cpd. No. 127) as a pale brown gum (61 mg, 44%). (LC/MS; m/z 346.2 [M+H]⁺)

### Example 53: Synthesis of N-((5-methyl-1-(4-(trifluoromethyl)phenyl)-1,2,3,4-tetrahydroquinolin-3-yl)methyl)acrylamide (Cpd. No. 128)

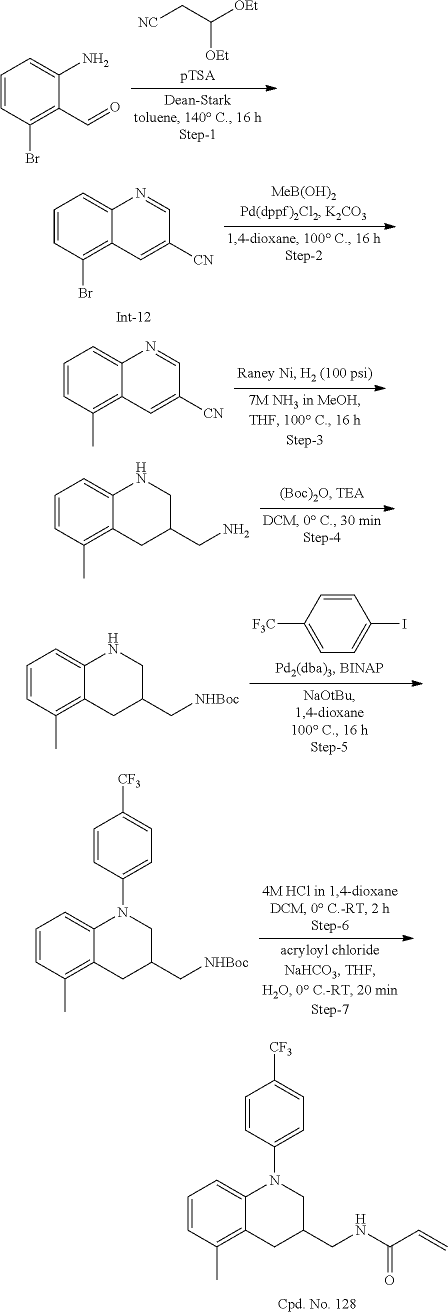

Step 1: A solution of 2-amino-6-bromobenzaldehyde (3.2 g, 16.1 mmol) in toluene (30 mL) was treated with pTSA (0.55 g, 3.22 mmol) and 3,3-diethoxypropanenitrile (2.7 g, 19.3 mmol) at RT under a nitrogen atmosphere. The reaction mixture was stirred at 140° C. in a Dean-Stark apparatus for 3 h. Progress of the reaction was monitored by TLC. TLC mobile phase: 10% EtOAc in pet ether, RF: 0.26, TLC detection: UV. The reaction mixture was diluted with water (200 mL) and extracted with EtOAc (2×250 mL). The combined organic layer was dried over Na₂SO₄, filtered and concentrated under reduced pressure to afford crude product as a yellow gum (3.2 g, LC/MS 33%) which was purified by normal phase column chromatography (Grace) using 40 g column and gradient of 7% EtOAc in pet ether as an eluent to afford 5-bromoquinoline-3-carbonitrile (Int-12) as an off-white solid (1.6 g, 34%). (LC/MS; m/z 233.0 [M+H]⁺)

Step 2: A solution of 5-bromoquinoline-3-carbonitrile (Int-12) (1.7 g, 7.3 mmol, LC/MS 79%) (sealed tube) in 1,4-dioxane (18 mL) was treated with methyl boronic acid (524 mg, 8.76 mmol) and K₂CO₃ (2.5 g, 18.2 mmol). The reaction mixture was degassed with argon for 5 min, treated with Pd(dppf)Cl₂ (534 mg, 0.73 mmol) and stirred at 100° C. for 16 h. Progress of the reaction was monitored by TLC. TLC mobile phase: 20% EtOAc in pet ether, RF: 0.52, TLC detection: UV. The reaction mixture was diluted with ice water (40 mL) and extracted with EtOAc (3×15 mL). The organic layer was dried over Na₂SO₄ and evaporated under reduced pressure to afford crude product (2.0 g) which was purified by normal phase column chromatography (Combi) using 40 g column and gradient of 20% EtOAc in pet ether as an eluent to afford 5-methylquinoline-3-carbonitrile as an off-white solid (500 mg, 48%). (LC/MS; m/z 168.9 [M+H]⁺)

Step 3: A solution of 5-methylquinoline-3-carbonitrile (400 mg, 2.38 mmol, LC/MS 93%) in THF (10 mL) was treated with Raney Ni (800 mg) and 7M NH₃ in MeOH (1 mL) and the reaction mixture was stirred at 100° C. under a hydrogen atmosphere (100 psi) for 16 h. Progress of the reaction was monitored by TLC. TLC mobile phase: 10% MeOH in DCM, RF: 0.11, TLC detection: UV. The reaction mixture was cooled to RT, filtered through a celite pad and rinsed with EtOAc (150 mL). The filtrate was concentrated under reduced pressure to afford (5-methyl-1,2,3,4-tetrahydroquinolin-3-yl)methanamine as a pale yellow gum (400 mg, 55%). (LC/MS; m/z 177.1 [M+H]⁺)

Step 4: A solution of (5-methyl-1,2,3,4-tetrahydroquinolin-3-yl)methanamine (400 mg, 2.27 mmol, LC/MS 54%) in DCM (10 mL) was treated with TEA (278 mg, 2.72 mmol) and (Boc)₂O (247 mg, 1.13 mmol) dropwise at 0° C. under a nitrogen atmosphere. The reaction mixture was stirred at 0° C. for 30 min. Progress of the reaction was monitored by TLC. TLC mobile phase: 5% MeOH in DCM, RF: 0.69, TLC detection: UV. The reaction mixture was diluted with water (50 mL) and extracted with DCM (2×50 mL). The combined organic layer was dried over Na₂SO₄, filtered and concentrated under reduced pressure to afford crude product as a yellow gum (450 mg, LC/MS 48%) which was purified by normal phase column chromatography (Grace) using 24 g column and a gradient of 25% EtOAc in pet ether as an eluent to afford tert-butyl ((5-methyl-1,2,3,4-tetrahydroquinolin-3-yl)methyl)carbamate as an off-white solid (190 mg, 52%). (LC/MS; m/z 277.1 [M+H]⁺)

Step 5: A solution of tert-butyl ((5-methyl-1,2,3,4-tetrahydroquinolin-3-yl)methyl)carbamate (230 mg, 0.83 mmol, LC/MS 94%) in 1,4-dioxane (3 mL) (sealed tube) was treated with 1-iodo-4-(trifluoromethyl)benzene (453 mg, 1.66 mmol), NaOtBu (159 mg, 1.66 mmol) at RT and degassed with argon for 5 min. BINAP (103 mg, 1.66 mmol) and Pd₂(dba)₃ (76 mg, 0.083 mmol) were added and the reaction mixture was stirred at 100° C. for 16 h. Progress of the reaction was monitored by TLC. TLC mobile phase: 20% EtOAc in pet ether, RF: 0.61, TLC detection: UV. The reaction mixture was cooled to RT, diluted with water (50 mL) and extracted with EtOAc (2×100 mL). The combined organic layer was dried over Na₂SO₄, filtered and concentrated under reduced pressure to afford crude product as a brown gum (300 mg, LC/MS 35%) which was purified by normal phase column chromatography (Grace) using a 24 g column and a gradient of 10% EtOAc in pet ether as an eluent to afford tert-butyl((5-methyl-1-(4-(trifluoromethyl)phenyl)-1,2,3,4-tetrahydroquinolin-3yl)methyl)carbamate as a pale yellow gum (180 mg, 46%). (LC/MS; m/z 421.3 [M+H]⁺)

Steps 6-7: These steps were executed in a manner similar (use of appropriate reagents and purification methods known to the person skilled in the art) to Cpd. No. 084. Starting material (180 mg, 0.42 mmol, LC/MS 85%) yielded a pale yellow gum (140 mg, LC/MS 79%) which was purified by preparative HPLC method H5. The collected fractions were concentrated under reduced pressure and lyophilised to afford N-((5-methyl-1-(4-(trifluoromethyl)phenyl)-1,2,3,4-tetrahydroquinolin-3-yl)methyl)acrylamide (Cpd. No. 128) as a white solid (50 mg, 46%). (LC/MS; m/z 375.2 [M+H]⁺)

The following compounds were prepared in a manner similar to Cpd. No. 128 by using appropriate reagents and purification methods known to the person skilled in the art: Cpd. No. 129 and Cpd. No. 130. To prepare Cpd. No. 129, 2-benzyl-4,4,5,5-tetramethyl-1,3,2-dioxaborolane and K₃PO₄ in water and 1,4-dioxane was employed.

### Example 54: Synthesis of N-((6-methoxy-1-(4-(trifluoromethyl)phenyl)-1,2,3,4-tetrahydroquinolin-3-yl)methyl)acrylamide (Cpd. No. 131)

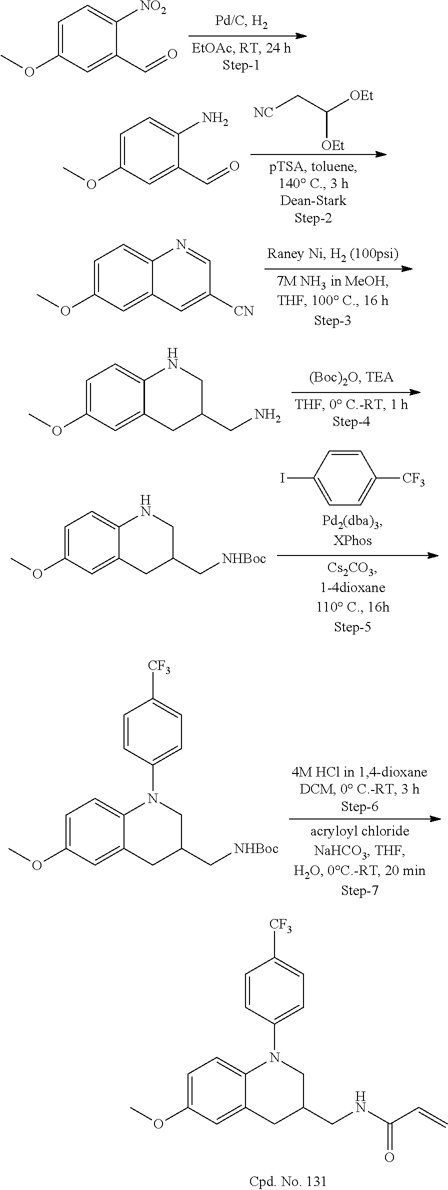

Step 1: A solution of 5-methoxy-2-nitrobenzaldehyde (2.9 g, 16 mmol) in EtOAc (25 mL) was treated with 10% Pd/C (800 mg) and stirred under a hydrogen atmosphere (balloon pressure) for 24 h at RT. Progress of the reaction was monitored by TLC. TLC mobile phase: 20% EtOAc in pet ether, RF: 0.2, TLC detection: UV. The reaction mixture was filtered through a celite pad and washed with EtOAc (35 mL). The filtrate was evaporated under reduced pressure to afford 2-amino-5-methoxybenzaldehyde as a yellow liquid (2.5 g) which was taken forward in the subsequent reaction step without further purification. (LC/MS; m/z 152.0 [M+H]⁺)

Step 2: A solution of 2-amino-5-methoxybenzaldehyde (2.5 g, 16.6 mmol) in toluene (25 mL) was treated with pTSA (630 mg, 3.3 mmol) and 3,3-diethoxypropanenitrile (2.8 g, 19.9 mmol) at RT under a nitrogen atmosphere. The reaction mixture was stirred at 140° C. in a Dean-Stark apparatus for 3 h. Progress of the reaction was monitored by TLC. TLC mobile phase: 20% EtOAc in pet ether, RF: 0.4, TLC detection: UV. The reaction mixture was diluted with ice water (75 mL) and extracted with EtOAc (2×20 mL). The organic layer was dried over Na₂SO₄, filtered and evaporated under reduced pressure to afford crude product (3.0 g) which was purified by flash chromatography (Grace) using a 40 g reveleris column and eluted with a 20% EtOAc in pet ether gradient to afford 6-methoxyquinoline-3-carbonitrile as a yellow solid (1.0 g, 40%). (LC/MS; m/z 185.0 [M+H]⁺)

Step 3: A solution of 6-methoxyquinoline-3-carbonitrile (400 mg, 2.17 mmol, LC/MS 90%) in THF (3 mL) was treated with Raney nickel (25 mg), NH₃ (7M in MeOH, 0.4 mL) and stirred under a hydrogen atmosphere (100 psi) in an autoclave for 16 h at 100° C. Progress of the reaction was monitored by TLC. TLC mobile phase: 20% EtOAc in pet ether. The reaction mixture was filtered through a celite pad and washed with MeOH (8 mL). The filtrate was evaporated under reduced pressure to afford (6-methoxy-1,2,3,4-tetrahydroquinolin-3-yl)methanamine as a yellow liquid (250 mg) which was taken forward in the subsequent reaction step without further purification. (LC/MS; m/z 193.1 [M+H]⁺)

Step 4: A solution of (6-methoxy-1,2,3,4-tetrahydroquinolin-3-yl)methanamine (250 mg, 1.3 mmol, LC/MS 18.8%) in THF (2.5 mL) was cooled to 0° C., treated with TEA (262 mg, 2.6 mmol) followed by a solution of (Boc)₂O (225 mg, 1.04 mmol) in THF (0.5 mL) under an argon atmosphere. The solution was stirred at RT for 1 h. Progress of the reaction was monitored by TLC. TLC mobile phase: 20% EtOAc in pet ether, RF: 0.5, TLC detection: UV. The reaction mixture was diluted with cold water (15 mL) and extracted with EtOAc (2×10 mL). The organic layer was separated, dried over Na₂SO₄ and evaporated under reduced pressure to afford crude product (300 mg) which was purified by flash chromatography (Grace) using a 24 g reveleris column and eluted with 20% EtOAc in pet ether gradient to afford tert-butyl ((6-methoxy-1,2,3,4-tetrahydroquinolin-3-yl)methyl)carbamate as an off white solid (100 mg). (LC/MS; m/z 293.1 [M+H]⁺)

Step 5: A solution of tert-butyl ((6-methoxy-1,2,3,4-tetrahydroquinolin-3-yl)methyl)carbamate (100 mg, 0.34 mmol, LC/MS 90%) in 1,4-dioxane (3 mL) was treated with Cs₂CO₃ (278 mg, 0.8 mmol) and degassed with argon for 5 min. The reaction mixture was treated with 1-iodo-4-(trifluoromethyl)benzene (139 mg, 0.51 mmol), XPhos (32.6 mg, 0.06 mmol), Pd₂(dba)₃ (31.3 mg, 0.03 mmol) under an argon atmosphere and stirred in a sealed tube at 110° C. for 16 h. Progress of the reaction was monitored by TLC. TLC mobile phase: 20% EtOAc in pet ether, RF: 0.48, TLC detection: UV. The reaction mixture was diluted with ice water (10 mL) and extracted with EtOAc (3×10 mL). The organic layer was dried over Na₂SO₄, filtered and evaporated under reduced pressure to afford crude product (110 mg, LC/MS 23%) which was purified by flash chromatography using a 12 g reveleris column and eluted with 20% EtOAc in pet ether gradient to afford tert-butyl ((6-methoxy-1-(4-(trifluoromethyl)phenyl)-1,2,3,4-tetrahydroquinolin-3-yl)methyl)carbamate as a pale yellow liquid (30 mg, 11%). (LC/MS; m/z 437.2 [M+H]⁺)

Steps 6-7: These steps were executed in a manner similar (use of appropriate reagents and purification methods known to the person skilled in the art) to Cpd. No. 084. Starting material (25 mg, 0.057 mmol, LC/MS 52%) yielded a pale yellow gum (25 mg, LC/MS 42%) which was purified by preparative HPLC method H3. The collected fractions were concentrated under reduced pressure and lyophilised to afford N-((6-methoxy-1-(4-(trifluoromethyl)phenyl)-1,2,3,4-tetrahydroquinolin-3-yl)methyl)acrylamide (Cpd. No. 131) as a white solid (3.2 mg, 28%). (LC/MS; m/z 391.3 [M+H]⁺)

### Example 55: Synthesis of N-methyl-N-(1-(4-(trifluoromethyl)phenyl)-1,2,3,4-tetrahydroquinolin-3-yl)acrylamide (Cpd. No. 132)

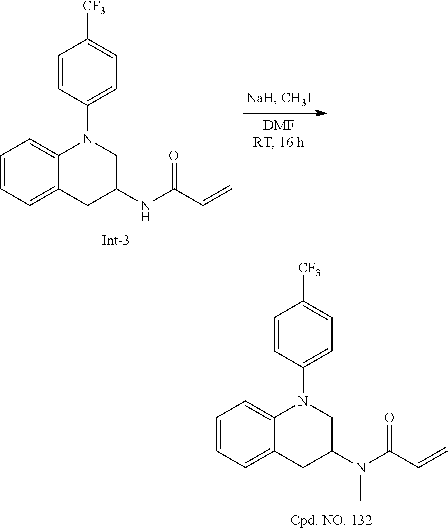 [SMILES: C=CC(=O)NC1Cc2ccccc2N(c2ccc(C(F)(F)F)cc2)C1 ; C=CC(=O)N(C)C1Cc2ccccc2N(c2ccc(C(F)(F)F)cc2)C1]

A stirred solution of Cpd. No. 001 (120 mg, 0.34 mmol) in DMF (3 mL) was treated with NaH (12.4 mg, 0.51 mmol), methyl iodide (98 mg, 0.69 mmol) at 0° C. under argon. The reaction mixture was stirred at RT for 16 hours. Progress of the reaction was monitored by TLC. TLC mobile phase: 30% EtOAc in pet ether, RF: 0.5, TLC detection: UV. The reaction mixture was diluted with cold water (20 mL) and extracted with EtOAc (2×10 mL), organic layer was separated, and dried over Na₂SO₄, filtered and filtrate was evaporated under reduced pressure to afford crude product. (130 mg, LC/MS 75%) as a yellow liquid. The crude compound was purified by preparative HPLC method H6. The collected fractions were evaporated by lyophilisation to afford N-methyl-N-(1-(4-(trifluoromethyl)phenyl)-1,2,3,4-tetrahydroquinolin-3-yl)acrylamide (Cpd. No. 132) as an off-white solid (15 mg, 22%). (LC/MS; m/z 361.2 [M+H]⁺)

The following compounds were prepared in a manner similar to Cpd. No. 132 by using appropriate reagents and purification methods known to the person skilled in the art: Cpd. No. 133, Cpd. No. 134, and Cpd. No. 135. Compound Cpd. No. 084 was used to prepare compounds Cpd. No. 134 and Cpd. No. 135.

### Examples 56-57: Synthesis of 3-acrylamido-1-(4-(trifluoromethyl)phenyl)-1,2,3,4-tetrahydroquinoline-5-carboxamide (Cpd. No. 136) and N-(5-(1H-tetrazol-5-yl)-1-(4-(trifluoromethyl)phenyl)-1,2,3,4-tetrahydroquinolin-3-yl)acrylamide (Cpd. No. 137)

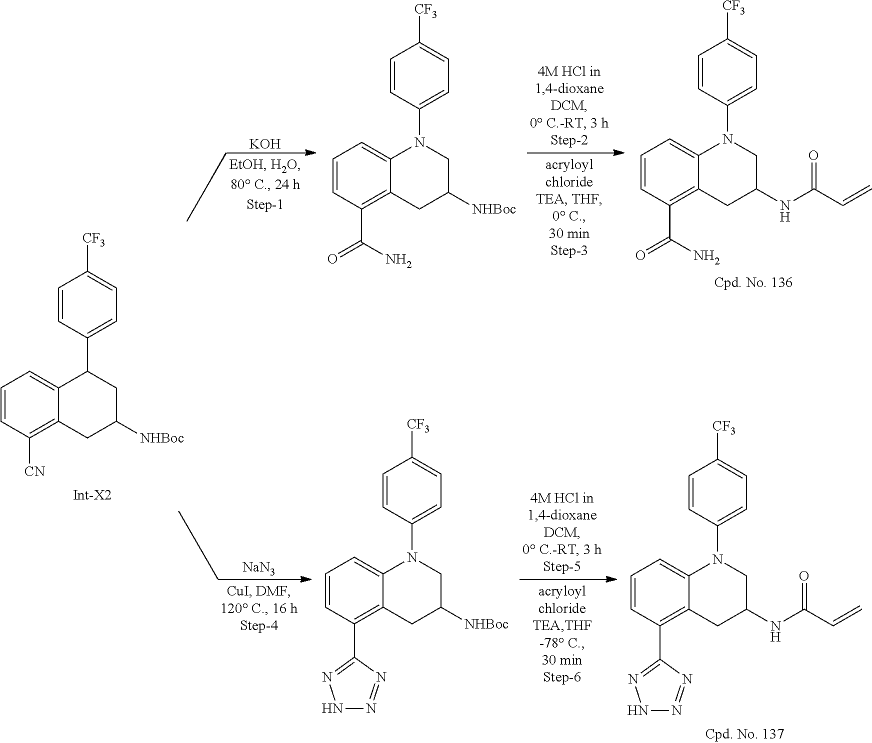

Step 1: A solution of Int-X2 (300 mg, 0.72 mmol) in EtOH (5.0 mL) and water (1.0 mL) was treated with KOH (201 mg, 3.59 mmol) at RT. The reaction mixture was stirred at 80° C. for 24 h. Progress of the reaction was monitored by TLC. TLC mobile phase: 70% EtOAc in pet ether, RF: 0.23, TLC detection: UV. TLC mobile phase: 10% MeOH in DCM, RF: 0.01, TLC detection: UV. The reaction mixture was diluted with water (20 mL) and extracted with EtOAc (2×50 mL). The organic fraction was separated, dried over anhydrous Na₂SO₄ and concentrated under reduced pressure to afford tert-butyl (5-carbamoyl-1-(4-(trifluoromethyl)phenyl)-1,2,3,4-tetrahydroquinolin-3-yl)carbamate as an off-white solid (300 mg, 71%, LC/MS 74%). (LC/MS; m/z 436.3 [M+H]⁺)

Steps 2-3: These steps were executed in a manner similar (use of appropriate reagents and purification methods known to the person skilled in the art) to Cpd. No. 001. Starting material (300 mg, 0.689 mmol, LC/MS 74%) yielded a pale yellow gum (200 mg, LC/MS 79%) which was purified by preparative HPLC method H2. The collected fractions were concentrated under reduced pressure and lyophilised to afford 3-acrylamido-1-(4-(trifluoromethyl)phenyl)-1,2,3,4-tetrahydroquinoline-5-carboxamide (Cpd. No. 136) as an off-white solid (123 mg, 62%). (LC/MS; m/z 390.2 [M+H]⁺)

Step 4: A solution of Int-X2 (700 mg, 1.67 mmol) in DMF (10.0 mL) (sealed tube) was treated with NaN₃ (218 mg, 3.35 mmol) and Cul (159 mg, 0.83 mmol) at RT. The reaction mixture was stirred at 120° C. for 16 h. Progress of the reaction was monitored by TLC. TLC mobile phase: 10% MeOH in DCM, RF: 0.11, TLC detection: UV. The reaction mixture was cooled to RT, poured into ice water (100 mL) and extracted with EtOAc (2×50 mL). The organic layer was washed with brine (50 mL), dried over anhydrous Na₂SO₄ and concentrated under reduced pressure to afford tert-butyl (5-(1H-tetrazol-5-yl)-1-(4-(trifluoromethyl)phenyl)-1,2,3,4-tetrahydroquinolin-3-yl)carbamate as a brown gum (800 mg, 84%, LC/MS 81%) which was taken forward in the subsequent reaction. (LC/MS; m/z 461.4 [M+H]⁺)

Steps 5-6: These steps were executed in a manner similar (use of appropriate reagents and purification methods known to the person skilled in the art) to Cpd. No. 001. Starting material (800 mg, 1.73 mmol, LC/MS 81%) yielded a brown solid (600 mg, LC/MS 54%) which was purified by preparative HPLC method H5. The collected fractions were concentrated under reduced pressure and lyophilised to afford N-(5-(1H-tetrazol-5-yl)-1-(4-(trifluoromethyl)phenyl)-1,2,3,4-tetrahydroquinolin-3-yl)acrylamide (Cpd. No. 137) as an off-white solid (59 mg, 7%). (LC/MS; m/z 415.3 [M+H]⁺)

### Examples 58-59: Synthesis of N-(5-(aminomethyl)-1-(4-(trifluoromethyl)phenyl)-1,2,3,4-tetrahydroquinolin-3-yl)acrylamide (Cpd. No. 138) and N-(5-(hydroxymethyl)-1-(4-(trifluoromethyl)phenyl)-1,2,3,4-tetrahydroquinolin-3-yl)acrylamide (Cpd. No. 139)

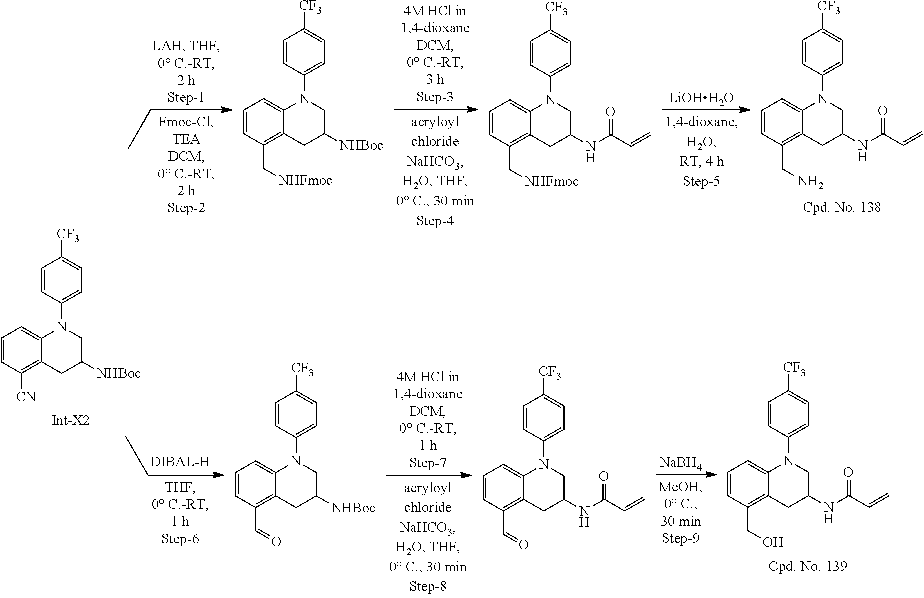

Step 1: A solution of Int-X2 (700 mg, 1.678 mmol) in THF (10 mL) was cooled to 0° C. and treated with LAH (2M in THF) (2.517 mL, 5.034 mmol) under a nitrogen atmosphere. The reaction mixture was stirred at RT for 2 h. Progress of the reaction was monitored by TLC. TLC mobile phase: 10% MeOH in DCM, RF: 0.12, TLC detection: UV. The reaction mixture was cooled to 0° C., quenched with sat. Na₂SO₄ solution (8 mL), diluted with water (50 mL), extracted with EtOAc (2×50 mL), dried over Na₂SO₄ and concentrated under reduced pressure to afford tert-butyl (5-(aminomethyl)-1-(4-(trifluoromethyl)phenyl)-1,2,3,4-tetrahydroquinolin-3-yl)carbamate (650 mg, 80%, LC/MS 87%). (LC/MS; m/z 422.3 [M+H]⁺)

Step 2: A solution of tert-butyl (5-(aminomethyl)-1-(4-(trifluoromethyl)phenyl)-1,2,3,4-tetrahydroquinolin-3-yl)carbamate (650 mg, 1.543 mmol, LC/MS 87%) in DCM (10 mL) was cooled to 0° C. and treated with TEA (0.216 mL, 1.543 mmol) and Fmoc-Cl (599 mg, 2.315 mmol). The reaction mixture was stirred under a nitrogen atmosphere at RT for 2 h.

Progress of the reaction was monitored by TLC. TLC mobile phase: 50% EtOAc in pet ether, RF: 0.50, TLC detection: UV. The reaction mixture was diluted with DCM (50 mL) and water (50 mL). The organic layer was separated and the aqueous layer was extracted with DCM (50 mL). The combined organic layer was washed with water (50 mL), dried over Na₂SO₄ and concentrated under reduced pressure to afford a pale brown solid (1.2 g, LC/MS 52%). The crude product was purified by normal phase column chromatography (Combi) using a 24 g column and a gradient of 9% EtOAc in pet ether as an eluent to afford tert-butyl (5-(((((9H-fluoren-9-yl)methoxy)carbonyl)amino)methyl)-1-(4-(trifluoromethyl)phenyl)-1,2,3,4-tetrahydroquinolin-3-yl)carbamate as a white solid (550 mg, 63%). (LC/MS; m/z 644.5 [M+H]⁺)

Steps 3-4: These steps were executed in a manner similar (use of appropriate reagents and purification methods known to the person skilled in the art) to Cpd. No. 001. Starting material (550 mg, 0.855 mmol) yielded a brown solid (500 mg, LC/MS 61%) which was purified by normal phase column chromatography (Combi) using a 24 g column and a gradient of 46% EtOAc in pet ether as an eluent to afford (9H-fluoren-9-yl)methyl((3-acrylamido-1-(4-(trifluoromethyl)phenyl)-1,2,3,4-tetrahydroquinolin-5-yl)methyl)carbamate as an off-white solid (450 mg, 67%, LC/MS 76%). (LC/MS; m/z 598.3 [M+H]⁺)

Step 5: A solution of (9H-fluoren-9-yl)methyl((3-acrylamido-1-(4-(trifluoromethyl)phenyl)-1,2,3,4-tetrahydroquinolin-5-yl)methyl)carbamate (430 mg, 0.720 mmol, LC/MS 76%) in 1,4-dioxane (10.0 mL) was cooled to 0° C. and treated with a solution of LiOH·H₂O (60 mg, 1.440 mmol) in water (2 mL). The reaction mixture was stirred at RT for 4 h. Progress of the reaction was monitored by TLC. TLC mobile phase: 10% MeOH in DCM, RF: 0.08, TLC detection: UV. The reaction mixture was diluted with water (50 mL) and EtOAc (50 mL). The organic layer was separated and the aqueous layer was extracted with EtOAc (50 mL). The combined organic layer was washed with water (50 mL), dried over Na₂SO₄ and concentrated under reduced pressure to afford a pale yellow solid (370 mg, LC/MS 65%). The crude product was purified by preparative HPLC method H4. The collected fractions were concentrated and lyophilized to afford N-(5-(aminomethyl)-1-(4-(trifluoromethyl)phenyl)-1,2,3,4-tetrahydroquinolin-3-yl)acrylamide (Cpd. No. 138) as a white solid (101 mg, 48%). (LC/MS; m/z 376.3 [M+H]⁺)

Step 6: A solution of Int-X2 (400 mg, 0.962 mmol, LC/MS 88%) in DCM (10 mL) was treated with DIBAL-H (1.0 M in toluene, 2.4 mL, 2.39 mmol) at 0° C. under a nitrogen atmosphere. The reaction mixture was stirred at RT for 1 h. Progress of the reaction was monitored by TLC. TLC mobile phase: 50% EtOAc in pet ether, RF: 0.67, TLC detection: UV. The reaction mixture was cooled to 0° C., quenched with sat. NH₄Cl (20 mL) and extracted with EtOAc (2×25 mL). The organic layer was dried over Na₂SO₄ and concentrated under reduced pressure to afford tert-butyl (5-formyl-1-(4-(trifluoromethyl)phenyl)-1,2,3,4-tetrahydroquinolin-3-yl)carbamate as a brown gum (370 mg, 82%, LC/MS 79%). (LC/MS; m/z 421.4 [M+H]⁺)

Steps 7-8: These steps were executed in a manner similar (use of appropriate reagents and purification methods known to the person skilled in the art) to Cpd. No. 001. Starting material (370 mg, 0.881 mmol, LC/MS 79%) yielded N-(5-formyl-1-(4-(trifluoromethyl)phenyl)-1,2,3,4-tetrahydroquinolin-3-yl)acrylamide as a pale brown gum (240 mg, 59%, LC/MS 64%). (LC/MS; m/z 375.2 [M+H]⁺)

Step 9: A solution of N-(5-formyl-1-(4-(trifluoromethyl)phenyl)-1,2,3,4-tetrahydroquinolin-3-yl)acrylamide (240 mg, 0.642 mmol, LC/MS 64%) in MeOH (10 mL) was treated with NaBH₄ (38 mg, 1.0 mmol) at 0° C. under a nitrogen atmosphere. The reaction mixture was stirred at 0° C. for 30 min. Progress of the reaction was monitored by TLC. TLC mobile phase: 30% EtOAc in pet ether, RF: 0.35, TLC detection: UV. The reaction mixture was diluted with ice water (10 mL), concentrated under reduced pressure and extracted with DCM (2×20 mL). The organic layer was dried over Na₂SO₄ and concentrated under reduced pressure to afford a pale brown solid (220 mg, LC/MS 61%). The crude product was purified by preparative HPLC method H2. The collected fractions were concentrated and lyophilized to afford N-(5-(hydroxymethyl)-1-(4-(trifluoromethyl)phenyl)-1,2,3,4-tetrahydroquinolin-3-yl)acrylamide (Cpd. No. 139) as an off-white solid (73 mg, 47%). (LC/MS; m/z 377.3 [M+H]⁺)

### Example 60: Synthesis of N-(5-(5-oxo-4,5-dihydro-1,2,4-oxadiazol-3-yl)-1-(4-(trifluoromethyl)phenyl)-1,2,3,4-tetrahydroquinolin-3-yl)acrylamide (Cpd. No. 166)

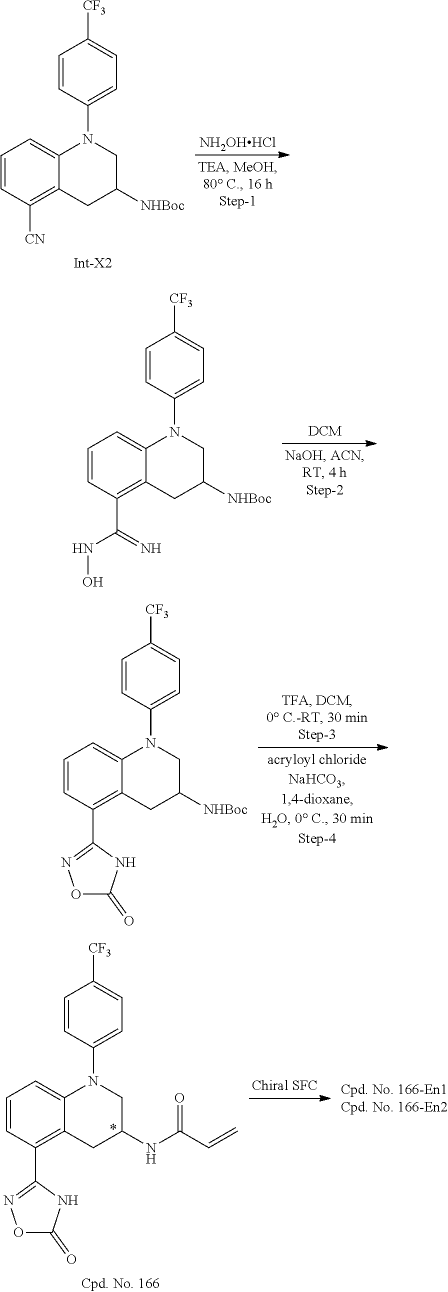

Step 1: A solution of Int-X2 (800 mg, 1.916 mmol) in methanol (10 mL) was treated with TEA (1.3 mL, 9.58 mmol) and hydroxylamine hydrochloride (665 mg, 9.58 mmol) at RT. The reaction mixture was stirred at 80° C. for 16 h. Progress of the reaction was monitored by TLC. TLC mobile phase: 5% MeOH in DCM, RF: 0.3, TLC detection: UV. The reaction mixture was concentrated under reduced pressure and extracted with EtOAc (2×50 mL). The organic layer was washed with brine (50 mL), dried over Na₂SO₄ and concentrated under reduced pressure to afford a pale brown gum (700 mg, LC/MS 47%). The crude product was purified by column chromatography using neutral alumina and a gradient of 5% MeOH in DCM as an eluent to afford tert-butyl (5-(N-hydroxycarbamimidoyl)-1-(4-(trifluoromethyl)phenyl)-1,2,3,4-tetrahydroquinolin-3-yl)carbamate as a pale yellow solid (200 mg, 20%, LC/MS 89%). (LC/MS; m/z 451.3 [M+H]⁺)

Step 2: A solution of tert-butyl (5-(N-hydroxycarbamimidoyl)-1-(4-(trifluoromethyl)phenyl)-1,2,3,4-tetrahydroquinolin-3-yl)carbamate (170 mg, 0.377 mmol, LC/MS 89%) in ACN (5 mL) was treated with NaOH (16 mg, 0.415 mmol) and dimethyl carbonate (0.05 mL, 0.566 mmol). The reaction was stirred at RT for 4 h under a nitrogen atmosphere. Progress of the reaction was monitored by TLC. TLC mobile phase: 10% MeOH in DCM, RF: 0.3, TLC detection: UV. The reaction mixture was concentrated under reduced pressure and extracted with EtOAc (40 mL). The organic layer was separated, washed with brine (10 mL), dried over Na₂SO₄ and concentrated under reduced pressure to afford crude product as an off-white solid (150 mg, 71%, LC/MS 76%). (LC/MS; m/z 477.1 [M+H]⁺)

Steps 3-4: These steps were executed in a manner similar (use of appropriate reagents and purification methods known to the person skilled in the art) to Cpd. No. 001. Starting material (130 mg, 0.273 mmol, LC/MS 76%) yielded a pale brown gum (150 mg, LC/MS 76%) which was purified by preparative HPLC method H3. The collected fractions were concentrated under reduced pressure and lyophilised to afford N-(5-(5-oxo-4,5-dihydro-1,2,4-oxadiazol-3-yl)-1-(4-(trifluoromethyl)phenyl)-1,2,3,4-tetrahydroquinolin-3-yl)acrylamide (Cpd. No. 166) as an off-white solid (12 mg, 13%). (LC/MS; m/z 431.3 [M+H]⁺) Chiral SFC purification: 300 mg of Cpd. No. 166 was purified by preparative SFC method K8 to afford Cpd. No. 166-En1 (30 mg) and Cpd. No. 166-En2 (25 mg), both as an off-white solid. (LC/MS; m/z 431.3 [M+H]⁺) The chiral purity of both enantiomers was assessed by analytic SFC method S8: Cpd. No. 166-En1, 99.0% ee; Cpd. No. 166-En2, 99.1% ee.

### Examples 61-62: Synthesis of 2-(3-acrylamido-1-(4-(trifluoromethyl)phenyl)-1,2,3,4-tetrahydroquinolin-5-yl)acetic acid (Cpd. No. 162) and N-(5-(cyanomethyl)-1-(4-(trifluoromethyl)phenyl)-1,2,3,4-tetrahydroquinolin-3-yl)acrylamide (Cpd. No. 164)

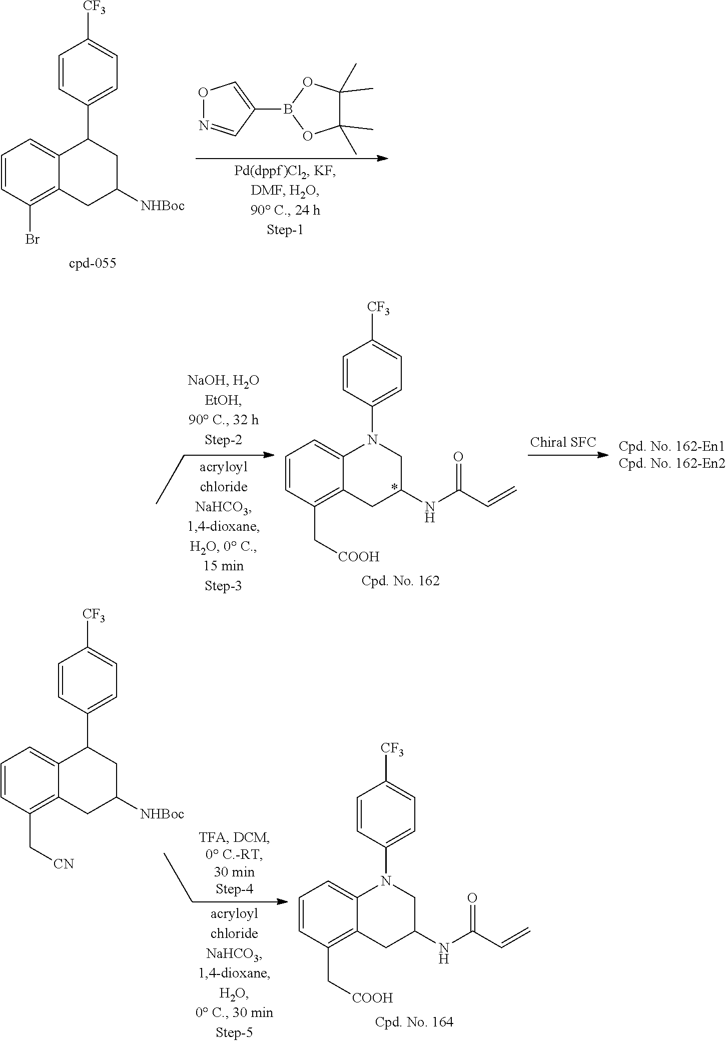 [SMILES: CC(C)(C)OC(=O)NC1Cc2c(Br)cccc2C(c2ccc(C(F)(F)F)cc2)C1 ; CC1(C)OB(c2cnoc2)OC1(C)C ; C=CC(=O)NC1Cc2c(CC(=O)O)cccc2N(c2ccc(C(F)(F)F)cc2)C1 ; CC(C)(C)OC(=O)NC1Cc2c(CC#N)cccc2C(c2ccc(C(F)(F)F)cc2)C1 ; C=CC(=O)NC1Cc2c(CC(=O)O)cccc2N(c2ccc(C(F)(F)F)cc2)C1]

Step 1: To a solution of Cpd. No. 055 (1.0 g, 2.12 mmol, LC/MS 81%) in DMF (30 ml) and water (5 ml) was added 4-(4,4,5,5-tetramethyl-1,3,2-dioxaborolan-2-yl)isoxazole (820 mg, 4.24 mmol) and KF (0.369 g, 6.36 mmol). The reaction mixture was purged with argon gas for 15 min, treated with Pd(dppf)Cl₂ (155 mg, 0.21 mmol) and stirred at 90° C. for 24 h. Progress of the reaction was monitored by TLC. TLC mobile phase: 20% EtOAc in pet ether, RF: 0.17, TLC detection: UV. The reaction mixture was cooled to RT and diluted with EtOAc (60 mL), filtered through a celite pad which was washed with EtOAc (60 mL). The filtrate was concentrated under reduced pressure to afford a brown gum (1.2 g, LC/MS 40%) which was purified by column chromatography (neutral alumina) and a gradient of 20% of EtOAc in pet ether as an eluent to afford tert-butyl (5-(cyanomethyl)-1-(4-(trifluoromethyl)phenyl)-1,2,3,4-tetrahydroquinolin-3-yl)carbamate as a pale yellow solid (350 mg, 40%, LC/MS 85%). (LC/MS; m/z 432.2 [M+H]⁺)

Step 2: A solution of tert-butyl (5-(cyanomethyl)-1-(4-(trifluoromethyl)phenyl)-1,2,3,4-tetrahydroquinolin-3-yl)carbamate (350 mg, 0.81 mmol, LC/MS 85%) in EtOH (5 ml) was treated with a 70% aq. NaOH solution (5 mL) and stirred at 90° C. for 32 h. Progress of the reaction was monitored by TLC. TLC mobile phase: 50% EtOAc in pet ether, RF: 0.14, TLC detection: UV. The reaction mixture was cooled to RT and concentrated under reduced pressure to affored sodium 2-(3-amino-1-(4-(trifluoromethyl)phenyl)-1,2,3,4-tetrahydroquinolin-5-yl)acetate as a pale yellow gum (500 mg, 53%, LC/MS 97%). (LC/MS; m/z 351.2 [M+H]⁺)

Step 3: This step was executed in a manner similar (use of appropriate reagents and purification methods known to the person skilled in the art) to step 4 towards Cpd. No. 001. Sodium 2-(3-amino-1-(4-(trifluoromethyl)phenyl)-1,2,3,4-tetrahydroquinolin-5-yl)acetate (450 mg, 1.206 mmol) yielded a pale yellow solid (250 mg, LC/MS 54%) which was purified by preparative HPLC method H12. The collected fractions were concentrated under reduced pressure and lyophilised to afford 2-(3-acrylamido-1-(4-(trifluoromethyl)phenyl)-1,2,3,4-tetrahydroquinolin-5-yl)acetic acid (Cpd. No. 162) as an off-white solid (50 mg, 10%). (LC/MS; m/z 405.3 [M+H]⁺) Chiral SFC purification: 37 mg of Cpd. No. 162 was purified by preparative SFC method K5 to afford Cpd. No. 162-En1 (11 mg) and Cpd. No. 162-En2 (8 mg), both as an off-white solid. (LC/MS; m/z 405.3 [M+H]⁺) The chiral purity of both enantiomers was assessed by analytic SFC method S5: Cpd. No. 162-En1, 99.4% ee; Cpd. No. 162-En2, 98.8% ee.

Steps 4-5: These steps were executed in a manner similar (use of appropriate reagents and purification methods known to the person skilled in the art) to Cpd. No. 001. Tert-butyl (5-(cyanomethyl)-1-(4-(trifluoromethyl)phenyl)-1,2,3,4-tetrahydroquinolin-3-yl)carbamate (100 mg, 0.232 mmol, LC/MS 86%) yielded a pale brown gum (120 mg, LC/MS 64%) which was purified by preparative HPLC method H3. The collected fractions were concentrated under reduced pressure and lyophilised to afford N-(5-(cyanomethyl)-1-phenyl-1,2,3,4-tetrahydroquinolin-3-yl)acrylamide (Cpd. No. 164) as an off-white solid (15 mg, 19%). (LC/MS; m/z 386.1 [M+H]⁺)

### Examples 63-64: Synthesis of 3-acrylamido-N-(methylsulfonyl)-1-(4-(trifluoromethyl)phenyl)-1,2,3,4-tetrahydroquinoline-5-carboxamide (Cpd. No. 160) and 3-acrylamido-N-cyano-1-(4-(trifluoromethyl)phenyl)-1,2,3,4-tetrahydroquinoline-5-carboxamide (Cpd. No. 169)

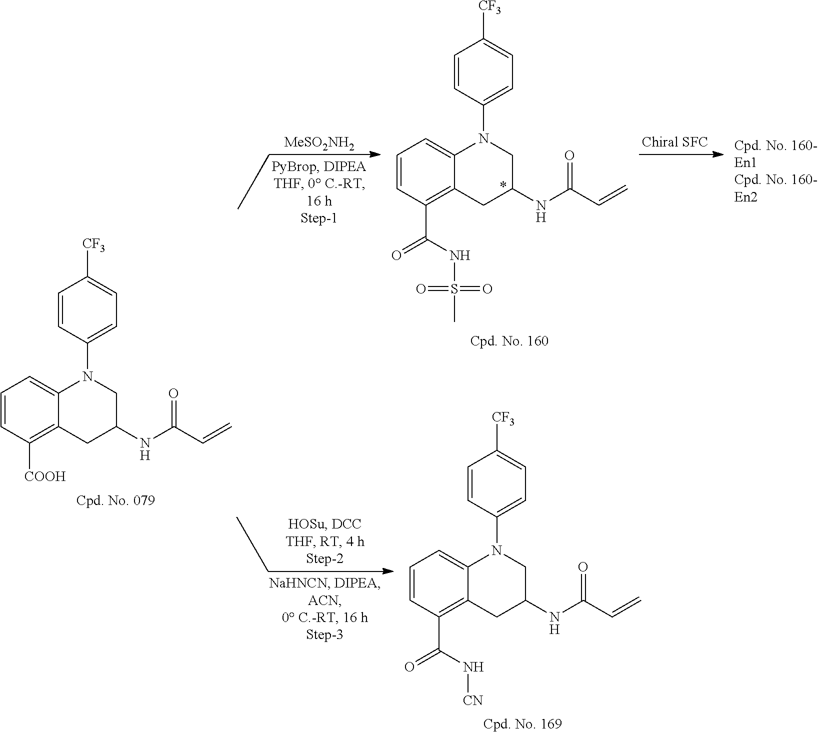 [SMILES: "MeSO2NH2" ; C=CC(=O)NC1Cc2c(C(=O)O)cccc2N(c2ccc(C(F)(F)F)cc2)C1 ; C=CC(=O)NC1Cc2c(C(=O)NS(C)(=O)=O)cccc2N(c2ccc(C(F)(F)F)cc2)C1 ; C=CC(=O)NC1Cc2c(C(=O)NC#N)cccc2N(c2ccc(C(F)(F)F)cc2)C1]

Step 1: A solution of Cpd. No. 079 (60 mg, 0.154 mmol, LC/MS 70%), PyBroP (179 mg, 0.384 mmol) and DIPEA (0.134 mL, 0.769 mmol) in THF (10 mL) was stirred at 0° C. for 5 min, then treated with methanesulfonamide (30 mg, 0.307 mmol). The reaction mixture was stirred at RT under a nitrogen atmosphere for 16 h. Progress of the reaction was monitored by TLC. TLC mobile phase: 10% MeOH in DCM, RF: 0.41, TLC detection: UV. The reaction mixture was diluted with ice water (15 mL) and the product was extracted with EtOAc (50 mL). The organic layer was dried over anhydrous Na₂SO₄, filtered and concentrated under reduced pressure to afford a brown gum (100 mg, LC/MS 33%) which was purified by preparative HPLC method H11. The collected fractions were lyophilized to afford 3-acrylamido-N-(methylsulfonyl)-1-(4-(trifluoromethyl)phenyl)-1,2,3,4-tetrahydroquinoline-5-carboxamide (Cpd. No. 160) as an off-white solid (20 mg, 40%). (LC/MS; m/z 468.2 [M+H]⁺) Chiral SFC purification: 117 mg of Cpd. No. 160 was purified by preparative SFC method K6 to afford Cpd. No. 160-En1 (23 mg) and Cpd. No. 160-En2 (31 mg), both as an off-white solid. (LC/MS; m/z 468.2 [M+H]⁺) The chiral purity of both enantiomers was assessed by analytic SFC method S8: Cpd. No. 160-En1, 99.9% ee; Cpd. No. 160-En2, 99.9% ee.

Step 2: A solution of Cpd. No. 079 (200 mg, 0.512 mmol) in THF (10 mL) was treated with HOSu (88 mg, 0.769 mmol) and DCC (204 mg, 0.769 mmol) and stirred at RT under a nitrogen atmosphere for 4 h. Progress of the reaction was monitored by TLC. TLC mobile phase: 10% MeOH in DCM. RF: 0.56, TLC detection: UV. The reaction mixture was filtered through a celite pad, the filtrate was concentrated under reduced pressure to afford an off-white solid (300 mg) which was passed through silica gel (100-200 mesh) using 2% MeOH in DCM as an eluent to afford 2,5-dioxopyrrolidin-1-yl-3-acrylamido-1-(4-(trifluoromethyl)phenyl)-1,2,3,4-tetrahydro-quinoline-5-carboxylate as an off-white solid (250 mg, 81%, LC/MS 81%). (LC/MS; m/z 488.4 [M+H]⁺)

Step 3: A solution of 2,5-dioxopyrrolidin-1-yl-3-acrylamido-1-(4-(trifluoromethyl)phenyl)-1,2,3,4-tetrahydroquinoline-5-carboxylate (250 mg, LC/MS 81%) in ACN (10 mL) was treated with DIPEA (0.18 mL, 1.02 mmol) and sodium hydrogencyanamide (40 mg, 0.615 mmol) at 0° C. The reaction mixture was stirred at RT for 16 h. The mixture was concentrated under reduced pressure, diluted with water (20 mL) and extracted with EtOAc (2×50 mL). The organic layer was washed with brine solution (10 mL), dried over anhydrous Na₂SO₄ and concentrated under reduced pressure to afford a pale brown solid (200 mg, LC/MS 88%) which was purified by preparative HPLC method H5. The collected fractions were lyophilized to afford 3-acrylamido-N-cyano-1-(4-(trifluoromethyl)phenyl)-1,2,3,4-tetrahydroquinoline-5-carboxamide (Cpd. No. 169) as a pale brown solid (30 mg, 17%). (LC/MS; m/z 415.3 [M+H]⁺)

Compound Cpd. No. 165 was prepared from Cpd. No. 162 in a manner similar to Cpd. No. 160 by using appropriate reagents and purification methods known to the person skilled in the art.

### Synthesis of 2-bromo-3-methyl-4-nitropyridine (Int-X3) and 6-methoxy-2-methyl-3-nitropyridine (Int-X4)

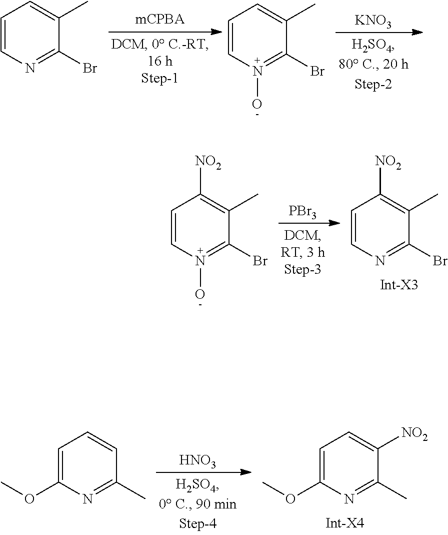

Step 1: To a solution of 2-bromo-3-methylpyridine (100 g, 581 mmol) in DCM (1 L) was added mCPBA (160.5 g, 930 mmol) portionwise (20 min) at 0° C. The reaction mixture was stirred at RT for 16 h. Progress of the reaction was monitored by TLC. TLC mobile phase: EtOAc, RF: 0.16, TLC detection: UV. The reaction mixture was concentrated and solids were filtered. The filtrate was concentrated under reduced pressure to afford a yellow solid (150 g, LC/MS 64%). The crude product was purified by flash column chromatography using silica gel (100-200 mesh) and a gradient of 70% EtOAc in pet ether as an eluent to afford 2-bromo-3-methylpyridine 1-oxide as an off-white solid (91 g, 84%). (LC/MS; m/z 188.0 [M+H]⁺)

Step 2: To a solution of 2-bromo-3-methylpyridine 1-oxide (91 g, 484 mmol) in H₂SO₄ (910 mL) was added KNO₃ (73.4 g, 726 mmol) portionwise (30 min) at 0° C. The reaction mixture was stirred at 80° C. for 20 h. Progress of the reaction was monitored by TLC. TLC mobile phase: 50% EtOAc in pet ether, RF: 0.56, TLC detection: UV. The reaction mixture was quenched with ice water (1 L) and extracted with EtOAc (2×1 L). The organic layer was washed with sat. NaHCO₃ (1 L), dried over Na₂SO₄ and concentrated under reduced pressure to afford a yellow solid (41.5 g, LC/MS 84%). The crude product was purified by flash column chromatography using silica gel (100-200 mesh) and a gradient of 19% EtOAc in pet ether as an eluent to afford 2-bromo-3-methyl-4-nitropyridine 1-oxide as a pale yellow solid (36.6 g, 32%). (LC/MS; m/z 233.0 [M+H]⁺)

Step 3: To a solution of 2-bromo-3-methyl-4-nitropyridine 1-oxide (36.6 g, 157.8 mmol) in DCM (366 mL) was added PBr₃ (44.8 mL, 473.3 mmol) dropwise (20 min) at 0° C. The reaction mixture was stirred at RT for 3 h. Progress of the reaction was monitored by TLC. TLC mobile phase: 50% EtOAc in pet ether, RF: 0.61, TLC detection: UV. The reaction mixture was quenched with sat. NaHCO₃ and extracted with DCM (2×300 mL). The combined organic layers were washed with water (300 mL), dried over Na₂SO₄ and concentrated under reduced pressure to afford a yellow solid (32.6 g, LC/MS 71%). The crude product was purified by flash column chromatography using silica gel (100-200 mesh) and a gradient of 12% EtOAc in pet ether as an eluent to afford 2-bromo-3-methyl-4-nitropyridine (Int-X3) as a pale yellow solid (24.7 g, 60%, LC/MS 83%). (LC/MS; m/z 216.9 [M+H]⁺)

Step 4: A solution of 2-methoxy-6-methylpyridine (5 g, 40.6 mmol) in conc. HNO₃ (7.5 mL, 183 mmol) was treated with conc. H₂SO₄ (18 mL, 328 mmol) at 0° C. The reaction mixture was stirred at RT for 90 min. Progress of the reaction was monitored by TLC. TLC mobile phase: 2% EtOAc in pet ether, RF: 0.48, TLC detection: UV. The reaction mixture was quenched with ice water (50 mL) and the obtained solids were filtered and dried under vacuum to afford 6-methoxy-2-methyl-3-nitropyridine (Int-X4) as an off-white solid (3.5 g, 51%). ¹H NMR (300 MHz, CDCl₃) b ppm: 8.27 (d, 1H), 6.67 (d, 1H), 4.01 (s, 3H), 2.82 (s, 3H)

### Synthesis of tert-butyl (6-bromo-2-oxo-1,2,3,4-tetrahydro-1,5-naphthyridin-3-yl)carbamate (Int-X5)

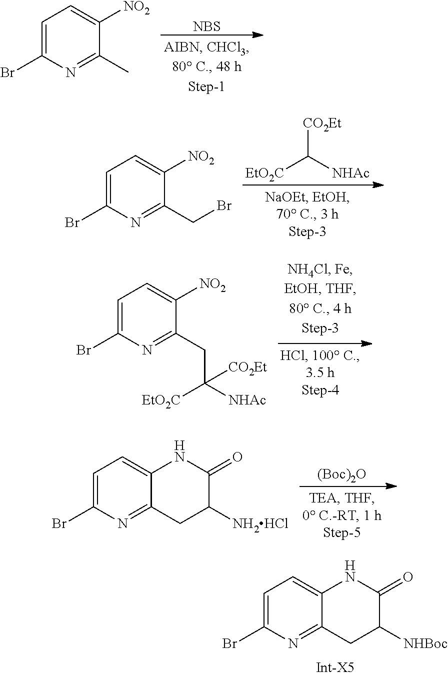

Steps 1-5: These steps were executed in a manner similar (use of appropriate reagents and purification methods known to the person skilled in the art) to steps 1-5 towards Cpd. No. 055. 6-bromo-2-methyl-3-nitropyridine (25 g, 115.7 mmol) yielded crude product (4 g) which was purified by normal phase column chromatography using a 24 g reveleris column (Combi) and a gradient of 50% EtOAc in pet ether as an eluent to afford tert-butyl (6-bromo-2-oxo-1,2,3,4-tetrahydro-1,5-naphthyridin-3-yl)carbamate (Int-X5) as an off-white solid (3.2 g, 7%, LC/MS 85%). (LC/MS; m/z 342.2 [M+H]⁺) The intermediates Int-X6 and Int-X7 were prepared from Int-X3 and Int-X4, respectively in a manner similar (use of appropriate reagents and purification methods known to the person skilled in the art) to Int-X5:

| Cpd. | | [M + H]⁺ |
|---|---|---|
| Nr. | Structure | (m/z) |
| Int-X6 | 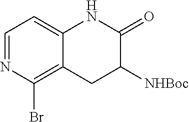 [SMILES: CC(C)(C)OC(=O)NC1Cc2c(ccnc2Br)NC1=O] | 342.1 |
| Int-X7 | 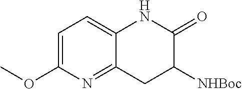 [SMILES: COc1ccc2c(n1)CC(NC(=O)OC(C)(C)C)C(=O)N2] | 438.3 |

### Synthesis of tert-butyl (6-bromo-1-(4-(trifluoromethyl)phenyl)-1,2,3,4-tetrahydro-1,5-naphthyridin-3-yl)carbamate (Int-X8)

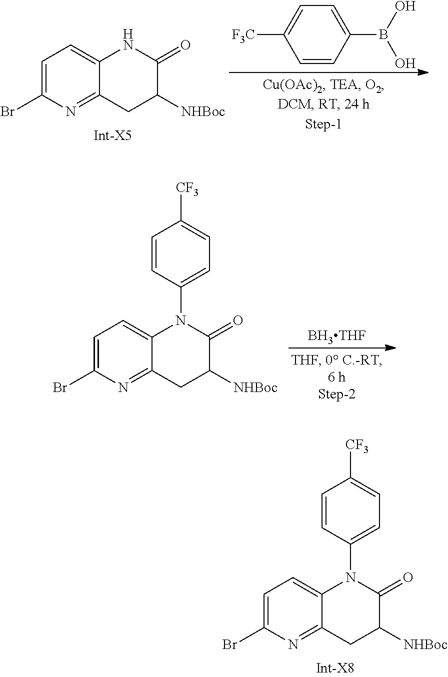 [SMILES: CC(C)(C)OC(=O)NC1Cc2nc(Br)ccc2NC1=O ; OB(O)c1ccc(C(F)(F)F)cc1 ; CC(C)(C)OC(=O)NC1Cc2nc(Br)ccc2N(c2ccc(C(F)(F)F)cc2)C1=O ; CC(C)(C)OC(=O)NC1Cc2nc(Br)ccc2N(c2ccc(C(F)(F)F)cc2)C1=O ; "BH3·THF"]

Steps 1-2: These steps were executed in a manner similar (use of appropriate reagents and purification methods known to the person skilled in the art) to steps 6-7 towards Cpd. No. 057. Int-X5 (3.2 g, 9.4 mmol, LC/MS 85%) yielded crude product (1.2 g) which was purified by normal phase column chromatography using a 24 g reveleris column (Combi) and a gradient of 15% EtOAc in pet ether as an eluent to afford tert-butyl (6-bromo-1-(4-(trifluoromethyl)phenyl)-1,2,3,4-tetrahydro-1,5-naphthyridin-3-yl)carbamate (Int-X8) as a pale green solid (500 mg, 11%, LC/MS 87%). (LC/MS; m/z 472.2 [M+H]⁺)

Intermediate Int-X9 was prepared from Int-X7 in a manner similar (use of appropriate reagents and purification methods known to the person skilled in the art) to Int-X8:

| Cpd. | | [M + H]⁺ |
|---|---|---|
| Nr. | Structure | (m/z) |
| Int-X9 | 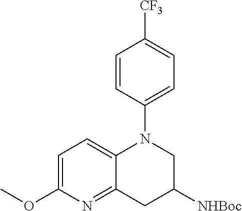 [SMILES: COc1ccc2c(n1)CC(NC(=O)OC(C)(C)C)CN2c1ccc(C(F)(F)F)cc1] | 424.3 |

### Synthesis of tert-butyl (5-bromo-1-(4-(trifluoromethyl)phenyl)-1,2,3,4-tetrahydro-1,6-naphthyridin-3-yl)carbamate (Int-X10)

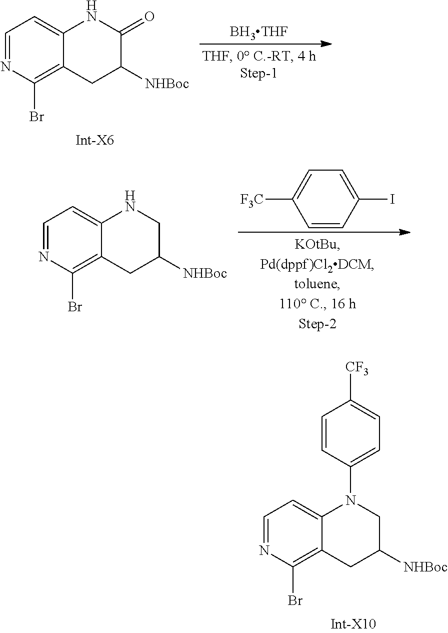 [SMILES: CC(C)(C)OC(=O)NC1Cc2c(ccnc2Br)NC1=O ; CC(C)(C)OC(=O)NC1CNc2ccnc(Br)c2C1 ; FC(F)(F)c1ccc(I)cc1 ; CC(C)(C)OC(=O)NC1Cc2c(ccnc2Br)N(c2ccc(C(F)(F)F)cc2)C1 ; "BH3·THF"]

Step 1: This step was executed in a manner similar (use of appropriate reagents and purification methods known to the person skilled in the art) to step 4 towards Cpd. No. 082. Int-X6 (500 mg, 1.46 mmol, LC/MS 91%) yielded crude product (500 mg, LC/MS 25%) which was purified by normal phase column chromatography using a 24 g column (Grace) and a gradient of 40% EtOAc in pet ether as an eluent to afford tert-butyl (5-bromo-1,2,3,4-tetrahydro-1,6-naphthyridin-3-yl)carbamate as an off-white solid (144 mg, 32%). (LC/MS; m/z 328.2 [M+H]⁺)

Step 2: To a solution of tert-butyl (5-bromo-1,2,3,4-tetrahydro-1,6-naphthyridin-3-yl)carbamate (500 mg, 0.153 mmol, LC/MS 93%) in toluene (10 mL) was added 1-iodo-4-(trifluoromethyl)benzene (0.073 mL, 0.765 mmol) and KOtBu (86 mg, 0.765 mmol). The solution was degassed with argon for 15 min and treated with Pd(dppf)Cl₂·DCM (31 mg, 0.038 mmol). The reaction mixture was stirred at 110° C. for 16 h (sealed tube). Progress of the reaction was monitored by TLC. TLC mobile phase: 30% EtOAc in pet ether, RF: 0.55, TLC detection: UV. The reaction mixture was cooled to RT and diluted with EtOAc (50 mL) and water (50 mL). The organic layer was separated and washed with water (50 mL), dried over Na₂SO₄ and concentrated under reduced pressure to afford a pale brown solid (600 mg, LC/MS 18%). The crude product was purified by normal phase column chromatography using a 24 g column and a gradient of 8% EtOAc in pet ether as an eluent to afford tert-butyl (5-bromo-1-(4-(trifluoromethyl)phenyl)-1,2,3,4-tetrahydro-1,6-naphthyridin-3-yl)carbamate (Int-X10) as a pale brown solid (180 mg, 25%, LC/MS 95%). (LC/MS; m/z 472.2 [M+H]⁺)

### Example 65: Synthesis of N-(6-amino-1-(4-(trifluoromethyl)phenyl)-1,2,3,4-tetrahydro-1,5-naphthyridin-3-yl)acrylamide (Cpd. No. 140)

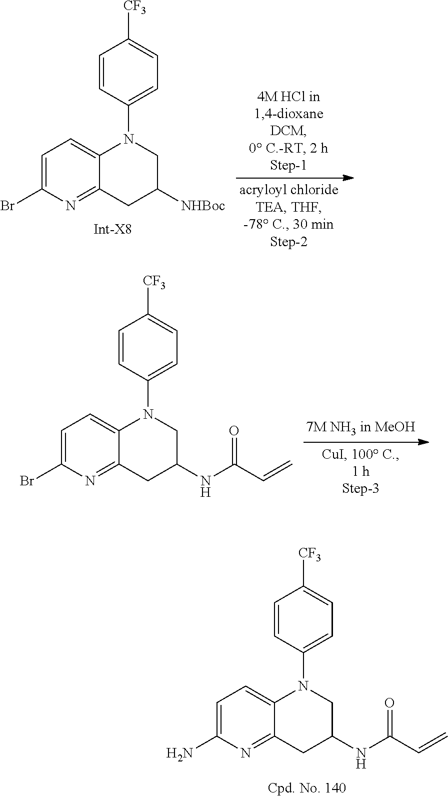 [SMILES: CC(C)(C)OC(=O)NC1Cc2nc(Br)ccc2N(c2ccc(C(F)(F)F)cc2)C1 ; C=CC(=O)NC1Cc2nc(Br)ccc2N(c2ccc(C(F)(F)F)cc2)C1 ; C=CC(=O)NC1Cc2nc(N)ccc2N(c2ccc(C(F)(F)F)cc2)C1]

Steps 1-2: These steps were executed in a manner similar (use of appropriate reagents and purification methods known to the person skilled in the art) to Cpd. No. 001. Int-X8 (500 mg, 1.05 mmol, LC/MS 87%) yielded a pale brown gum (450 mg, LC/MS 64%) which was purified by normal phase chromatography using a 24 g reveleris column (Combi) and a gradient of 13% EtOAc in pet ether as an eluent to afford N-(6-bromo-1-(4-(trifluoromethyl)phenyl)-1,2,3,4-tetrahydro-1,5-naphthyridin-3-yl)acrylamide as an off-white solid (300 mg, LC/MS 74%). (LC/MS; m/z 426.2 [M+H]⁺)

Step 3: To a solution of N-(6-bromo-1-(4-(trifluoromethyl)phenyl)-1,2,3,4-tetrahydro-1,5-naphthyridin-3-yl)acrylamide (200 mg, 0.47 mmol, LC/MS 74%) in 7M NH₃ in MeOH (10 mL) was added Cul (45 mg, 0.23 mmol). The reaction mixture was stirred at 100° C. for 1 h (sealed tube). Progress of the reaction was monitored by TLC. TLC mobile phase: 10% MeOH in DCM, RF: 0.46, TLC detection: UV. The reaction mixture was cooled to RT, concentrated under reduced pressure to afford a black solid (150 mg) which was purified by preparative HPLC method H3. The collected fractions were concentrated under reduced pressure and lyophilised to afford N-(6-amino-1-(4-(trifluoromethyl)phenyl)-1,2,3,4-tetrahydro-1,5-naphthyridin-3-yl)acrylamide as an off-white solid (15 mg, 12%). (LC/MS; m/z 363.3 [M+H]⁺)

Compound Cpd. No. 146 was prepared from Int-X10 in a manner similar to Cpd. No. 140 by using appropriate reagents and purification methods known to the person skilled in the art.

### Example 66: Synthesis of N-(6-methoxy-1-(4-(trifluoromethyl)phenyl)-1,2,3,4-tetrahydro-1,5-naphthyridin-3-yl)acrylamide (Cpd. No. 141)

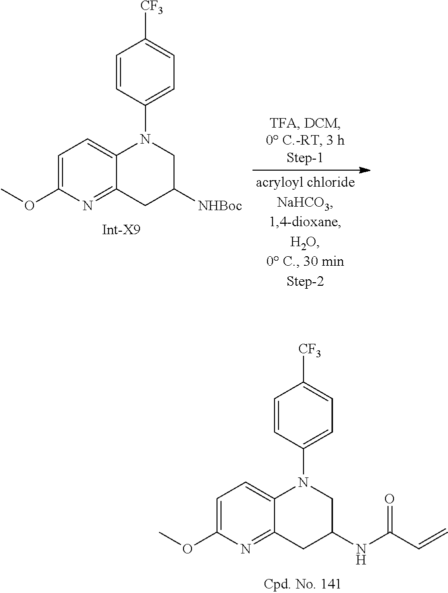 [SMILES: COc1ccc2c(n1)CC(NC(=O)OC(C)(C)C)CN2c1ccc(C(F)(F)F)cc1 ; C=CC(=O)NC1Cc2nc(OC)ccc2N(c2ccc(C(F)(F)F)cc2)C1]

Steps 1-2: These steps were executed in a manner similar (use of appropriate reagents and purification methods known to the person skilled in the art) to Cpd. No. 001. Int-X9 (150 mg, 0.354 mmol) yielded a pale brown gum (125 mg, LC/MS 71%) which was purified by preparative HPLC method H3. The collected fractions were concentrated under reduced pressure and lyophilised to afford N-(6-methoxy-1-(4-(trifluoromethyl)phenyl)-1,2,3,4-tetrahydro-1,5-naphthyridin-3-yl)acrylamide (Cpd. No. 141) as an off-white solid (28 mg, 21%). (LC/MS; m/z 378.2 [M+H]⁺)

### Example 67: Synthesis of N-((5-amino-1-(4-(trifluoromethyl)phenyl)-1,2,3,4-tetrahydroquinolin-3-yl)methyl)acrylamide (Cpd. No. 153)

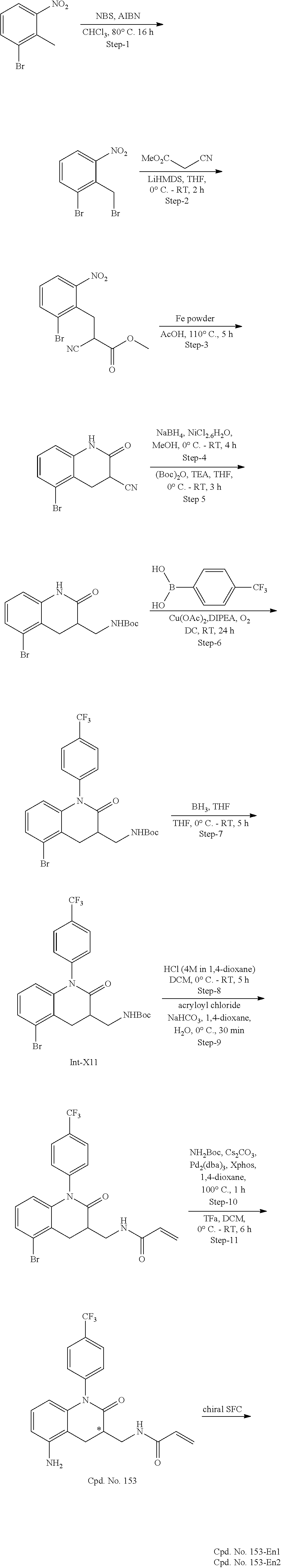

Step 1: This step was executed in a manner similar (use of appropriate reagents and purification methods known to the person skilled in the art) to Cpd. No. 055. 1-bromo-2-methyl-3-nitrobenzene (50 g, 231 mmol) yielded a brown gum (60 g) which was purified by silica gel column chromatography (60-120 mesh) and a gradient of 2% EtOAc in pet ether to afford 1-bromo-2-(bromomethyl)-3-nitrobenzene as a pale yellow solid (55 g, 80%). ¹H NMR (400 MHz, CDCl₃) b ppm: 7.86-7.89 (m, 2H), 7.35 (t, 1H), 4.89 (s, 2H)

Step 2: To a cooled solution (0° C.) of methyl 2-cyanoacetate (52 g, 176 mmol) in THF (500 mL) was added LiHMDS (352 mL, 353 mmol). The solution was stirred at 0° C. for 30 min and treated with a solution of 1-bromo-2-(bromomethyl)-3-nitrobenzene (52 g, 176 mmol) in THF (200 ml). The reaction mixture was stirred at RT for 2 h. Progress of the reaction was monitored by TLC. TLC mobile phase: 20% EtOAc in pet ether, RF: 0.25, TLC detection: UV. The reaction mixture was quenched with aq. NH₄Cl (1 L) at 0° C. and extracted with EtOAc (3×750 ml). The organic layer was washed with brine (800 mL), dried over Na₂SO₄, filtered and concentrated to afford crude product (65 g) which was purified by silica gel column chromatography (100-200 mesh) using a gradient of 5% of EtOAc in pet ether. The collected fractions were concentrated under reduced pressure to afford methyl 3-(2-bromo-6-nitrophenyl)-2-cyanopropanoate as an off-white gum (53 g, 88%, LC/MS 92%). (LC/MS; m/z 313.1 [M+H]⁺)

Step 3: A solution of methyl 3-(2-bromo-6-nitrophenyl)-2-cyanopropanoate (53 g, 155 mmol, LC/MS 92%) in AcOH (250 ml) was treated with iron powder (47.58 g, 778.63 mmol) and stirred at 110° C. for 5 h. Progress of the reaction was monitored by TLC. TLC mobile phase: 30% EtOAc in pet ether, RF: 0.32, TLC detection: UV. The reaction mixture was diluted with EtOAc (1 L) and filtered through a celite pad. The filtrate was washed with a sat. NaHCO₃ solution (3×500 mL), brine (500 mL), dried over Na₂SO₄ and concentrated under reduced pressure to afford a brown solid (51 g, LC/MS 71%). The crude product was purified by normal phase chromatography (Combi) using a 120 g reveleris column and a gradient of 20% EtOAc in pet ether to afford 5-bromo-2-oxo-1,2,3,4-tetrahydroquinoline-3-carbonitrile as an off-white solid (25 g, 63%). (LC/MS; m/z 251.1 [M+H]⁺)

Step 4: A solution of 5-bromo-2-oxo-1,2,3,4-tetrahydroquinoline-3-carbonitrile (25 g, 100 mmol) in MeOH (500 mL) was treated with NiCl₂.6H₂O (14 g, 61 mmol) and NaBH₄ (23 g, 600 mmol) at 0° C. The reaction mixture was stirred at RT for 4 h. Progress of the reaction was monitored by TLC. TLC mobile phase: 10% MeOH in DCM, RF: 0.2, TLC detection: UV. The reaction mixture was diluted with EtOAc (500 mL), filtered through a celite pad and concentrated under reduced pressure to afford 3-(aminomethyl)-5-bromo-3,4-dihydroquinolin-2(1H)-one (28 g, LC/MS 33%) which was taken forward in the subsequent reaction without further purification. (LC/MS; m/z 257.1 [M+H]⁺)

Steps 5-7: These steps were executed in a manner similar (use of appropriate reagents and purification methods known to the person skilled in the art) to steps 5-7 towards Cpd. No. 057. 3-(aminomethyl)-5-bromo-3,4-dihydroquinolin-2(1H)-one (16.8 g, 66.1 mmol, LC/MS 33%) yielded a brown gum (3.2 g) which was purified by column chromatography using neutral alumina and a gradient of 12% EtOAc in pet ether to afford tert-butyl ((5-bromo-1-(4-(trifluoromethyl)phenyl)-1,2,3,4-tetrahydroquinolin-3-yl)methyl)carbamate (Int-X11) as an off-white solid (2.4 g, 18%, LC/MS 81%). (LC/MS; m/z 485.2 [M+H]⁺)

Steps 8-10: These steps were executed in a manner similar (use of appropriate reagents and purification methods known to the person skilled in the art) to steps 1-3 towards Cpd. No. 074. Int-X11 (1.3 g, 2.75 mmol, LC/MS 81%) yielded a brown gum (760 mg) which was purified by normal phase chromatography (Combi) using a 40 g reveleris column and a gradient of 12% EtOAc in pet ether to afford tert-butyl (3-(acrylamidomethyl)-1-(4-(trifluoromethyl)phenyl)-1,2,3,4-tetrahydroquinolin-5-yl)carbamate as a pale yellow solid (400 mg, 36%, LC/MS 95%). (LC/MS; m/z 476.4 [M+H]⁺)

Step 11: A solution of tert-butyl (3-(acrylamidomethyl)-1-(4-(trifluoromethyl)phenyl)-1,2,3,4-tetrahydroquinolin-5-yl)carbamate (400 mg, 0.84 mmol, LC/MS 95%) in DCM (20 ml) was treated with TFA (0.32 mL, 0.84 mmol) at 0° C. The reaction mixture was stirred at RT for 6 h. Progress of the reaction was monitored by TLC. TLC mobile phase: 50% EtOAc in pet ether. The reaction mixture was cooled to 0° C., quenched with aq. NaHCO₃ (20 mL) and extracted with EtOAc (3×25 mL). The organic layer was washed with brine (25 mL), dried over Na₂SO₄ and concentrated to afford a brown gum (350 mg, LC/MS 64%). The crude product was purified by preparative HPLC method H2. The collected fractions were concentrated and lyophilized to afford N-((5-amino-1-(4-(trifluoromethyl)phenyl)-1,2,3,4-tetrahydroquinolin-3-yl)methyl)-acrylamide (Cpd. No. 153) as an off-white solid (95 mg, 31%). (LC/MS; m/z 376.3 [M+H]⁺) Chiral SFC purification: 65 mg of Cpd. No. 153 was further purified by preparative SFC method K4 to afford Cpd. No. 153-En1 (20 mg) and Cpd. No. 153-En2 (17 mg), both as an off-white solid. (LC/MS; m/z 376.3 [M+H]⁺). The chiral purity of both enantiomers was assessed by analytic SFC method S4: Cpd. No. 153-En1, 99.8% ee; Cpd. No. 153-En2, 99.2% ee.

Compound Cpd. No. 150 was prepared from Int-X11 in a manner similar to Cpd. No. 153 by using appropriate reagents and purification methods known to the person skilled in the art.

### Synthesis of tert-butyl ((5-(cyanomethyl)-1-(4-(trifluoromethyl)phenyl)-1,2,3,4-tetrahydroquinolin-3-yl)methyl)carbamate (Int-X12) and tert-butyl ((5-cyano-1-(4-(trifluoromethyl)phenyl)-1,2,3,4-tetrahydroquinolin-3-yl)methyl)carbamate (Int-X13)

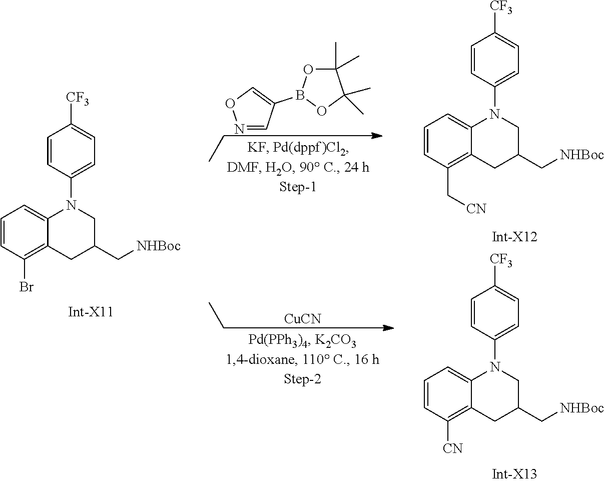 [SMILES: CC(C)(C)OC(=O)NCC1Cc2c(Br)cccc2N(c2ccc(C(F)(F)F)cc2)C1 ; CC1(C)OB(c2cnoc2)OC1(C)C ; "CuCN" ; CC(C)(C)OC(=O)NCC1Cc2c(CC#N)cccc2N(c2ccc(C(F)(F)F)cc2)C1 ; CC(C)(C)OC(=O)NCC1Cc2c(C#N)cccc2N(c2ccc(C(F)(F)F)cc2)C1]

Step 1: This step was executed in a manner similar (use of appropriate reagents and purification methods known to the person skilled in the art) to step 1 towards Cpd. No. 162. Int-X11 (1.0 g, 2.06 mmol, LC/MS 90%) yielded a brown gum (1.2 g, LC/MS 35%) which was purified by column chromatography using neutral alumina and a gradient of 10% EtOAc in pet ether to afford tert-butyl ((5-(cyanomethyl)-1-(4-(trifluoromethyl)phenyl)-1,2,3,4-tetrahydroquinolin-3-yl)methyl)carbamate (Int-X12) as a pale yellow solid (380 mg, 42%, LC/MS 91%). (LC/MS; m/z 446.1 [M+H]⁺)

Step 2: To a solution of Int-X11 (2.0 g, 4.12 mmol, LC/MS 90%) in 1,4-dioxane (40 mL) was added with CuCN (555 mg, 8.24 mmol), K₂CO₃ (3.41 g, 24.7 mmol). The mixture was degassed with argon for 15 min and treated with Pd(PPh₃)₄(476 mg, 0.412 mmol). The reaction mixture was stirred at 110° C. for 16 h (sealed tube). Progress of the reaction was monitored by TLC. TLC mobile phase: 20% EtOAc in pet ether, RF: 0.38, TLC detection: UV. The reaction mixture was cooled to RT, diluted with EtOAc (50 mL), filtered through a celite pad which was washed with EtOAc (25 mL). The filtrate was concentrated under reduced pressure to afford a pale yellow gum (2.2 g, LC/MS 41%) which was purified by normal phase column chromatography (Grace) using a 24 g column and a gradient of 15% EtOAc in pet ether as an eluent to afford tert-butyl ((5-cyano-1-(4-(trifluoromethyl)phenyl)-1,2,3,4-tetrahydroquinolin-3-yl)methyl)-carbamate (Int-X13) as off-white solid (1.6 g, 90%, LC/MS 98%). (LC/MS; m/z 432.2 [M+H]⁺)

### Example 68: Synthesis of N-((5-cyano-1-(4-(trifluoromethyl)phenyl)-1,2,3,4-tetrahydroquinolin-3-yl)methyl)acetamide (Cpd. No. 154)

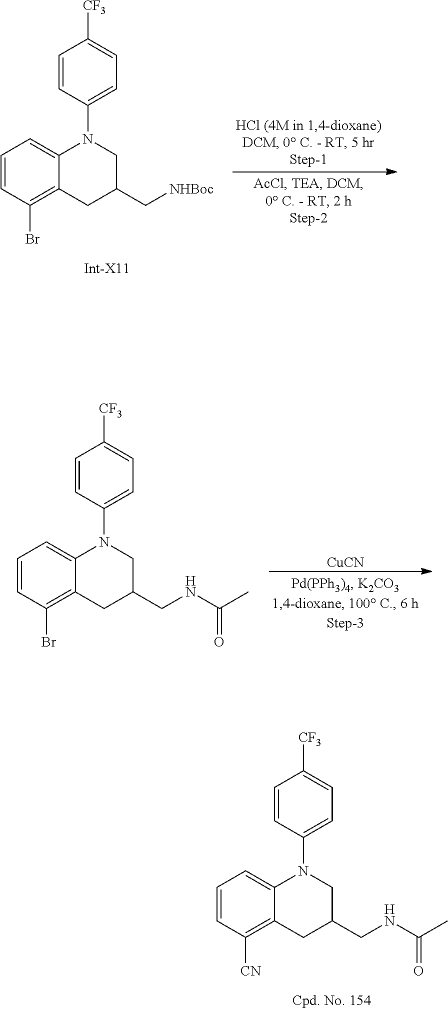 [SMILES: CC(C)(C)OC(=O)NCC1Cc2c(Br)cccc2N(c2ccc(C(F)(F)F)cc2)C1 ; CC(=O)NCC1Cc2c(Br)cccc2N(c2ccc(C(F)(F)F)cc2)C1 ; "CuCN" ; CC(=O)NCC1Cc2c(C#N)cccc2N(c2ccc(C(F)(F)F)cc2)C1]

Steps 1-2: These step was executed in a manner similar (use of appropriate reagents and purification methods known to the person skilled in the art) to steps 4-5 towards Cpd. No. 076. Int-X11 (3.9 g, 8.07 mmol, LC/MS 91%) yielded a brown gum (3.1 g, LC/MS 38%) which was purified by normal phase column chromatography (Combi) using a 24 g column and a gradient of 20% EtOAc in pet ether as an eluent to N-((5-bromo-1-(4-(trifluoromethyl)phenyl)-1,2,3,4-tetrahydroquinolin-3-yl)methyl)acetamide as a pale yellow gum (2.0 g, 63%, LC/MS 98%). (LC/MS; m/z 427.2 [M+H]⁺)

Step 3: This step was executed in a manner similar (use of appropriate reagents and purification methods known to the person skilled in the art) to Int-X13. N-((5-bromo-1-(4-(trifluoromethyl)phenyl)-1,2,3,4-tetrahydroquinolin-3-yl)methyl)acetamide (500 mg, 1.170 mmol) yielded a pale brown gum (700 mg, LC/MS 46%) which was purified by normal phase column chromatography (Grace) using a 24 g column and a gradient of 19% EtOAc in pet ether as an eluent to afford a pale yellow gum (300 mg, LC/MS 81%). 100 mg of compound was further purified by preparative HPLC method H4. The collected fractions were concentrated and lyophilized to afford N-((5-cyano-1-(4-(trifluoromethyl)phenyl)-1,2,3,4-tetrahydroquinolin-3-yl)methyl)acetamide (Cpd. No. 154) as an off-white solid (19 mg, 4%). (LC/MS; m/z 374.3 [M+H]⁺)

### Examples 69-70: Synthesis of 2-(3-(acrylamidomethyl)-1-(4-(trifluoromethyl)phenyl)-1,2,3,4-tetrahydroquinolin-5-yl)acetic acid (Cpd. No. 161) and N-((5-(cyanomethyl)-1-(4-(trifluoromethyl)phenyl)-1,2,3,4-tetrahydroquinolin-3-yl)methyl)acrylamide (Cpd. No. 163)

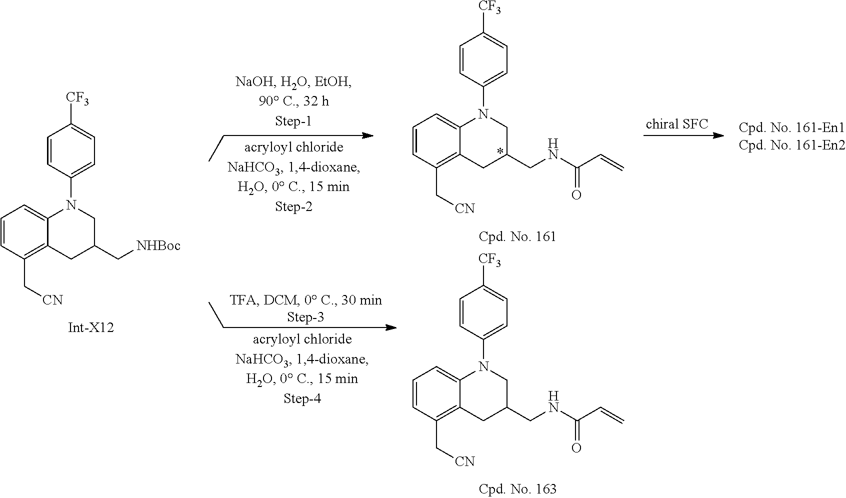 [SMILES: CC(C)(C)OC(=O)NCC1Cc2c(CC#N)cccc2N(c2ccc(C(F)(F)F)cc2)C1 ; "TFA, DCM, 0° C., 30 minStep-3" ; C=CC(=O)NCC1Cc2c(CC#N)cccc2N(c2ccc(C(F)(F)F)cc2)C1 ; C=CC(=O)NCC1Cc2c(CC#N)cccc2N(c2ccc(C(F)(F)F)cc2)C1]

Steps 1-2: These steps were executed in a manner similar (use of appropriate reagents and purification methods known to the person skilled in the art) to steps 2-3 towards Cpd. No. 162. Int-X12 (560 mg, 1.25 mmol, LC/MS 91%) yielded a pale yellow solid (200 mg, LC/MS 80%) which was purified by preparative HPLC method H12. The collected fractions were concentrated and lyophilized to afford 2-(3-(acrylamidomethyl)-1-(4-(trifluoromethyl)phenyl)-1,2,3,4-tetrahydroquinolin-5-yl)acetic acid (Cpd. No. 161) as an off-white solid (44 mg, 10%). (LC/MS; m/z 419.3 [M+H]⁺) Chiral SFC purification: 32 mg of Cpd. No. 161 was purified by preparative SFC method K5 to afford Cpd. No. 161-En1 (11 mg) and Cpd. No. 161-En2 (10 mg), both as an off-white solid. (LC/MS; m/z 419.3 [M+H]⁺). The chiral purity of both enantiomers was assessed by analytic SFC method S5: Cpd. No. 161-En1, 99.7% ee; Cpd. No. 161-En2, 95.9% ee.

Steps 3-4: These steps were executed in a manner similar (use of appropriate reagents and purification methods known to the person skilled in the art) to steps 4-5 towards Cpd. No. 164. Int-X12 (110 mg, 0.247 mmol, LC/MS 91%) yielded a pale brown gum (120 mg, LC/MS 61%) which was purified by preparative HPLC method H3. The collected fractions were concentrated and lyophilized to afford N-((5-(cyanomethyl)-1-(4-(trifluoromethyl)phenyl)-1,2,3,4-tetrahydroquinolin-3-yl)methyl)acrylamide (Cpd. No. 163) as an off-white solid (28 mg, 31%). (LC/MS; m/z 400.3 [M+H]⁺)

### Example 71: Synthesis of N-((5-((5-oxo-4,5-dihydro-1,2,4-oxadiazol-3-yl)methyl)-1-(4-(trifluoromethyl)phenyl)-1,2,3,4-tetrahydroquinolin-3-yl)methyl)acrylamide (Cpd. No. 168)

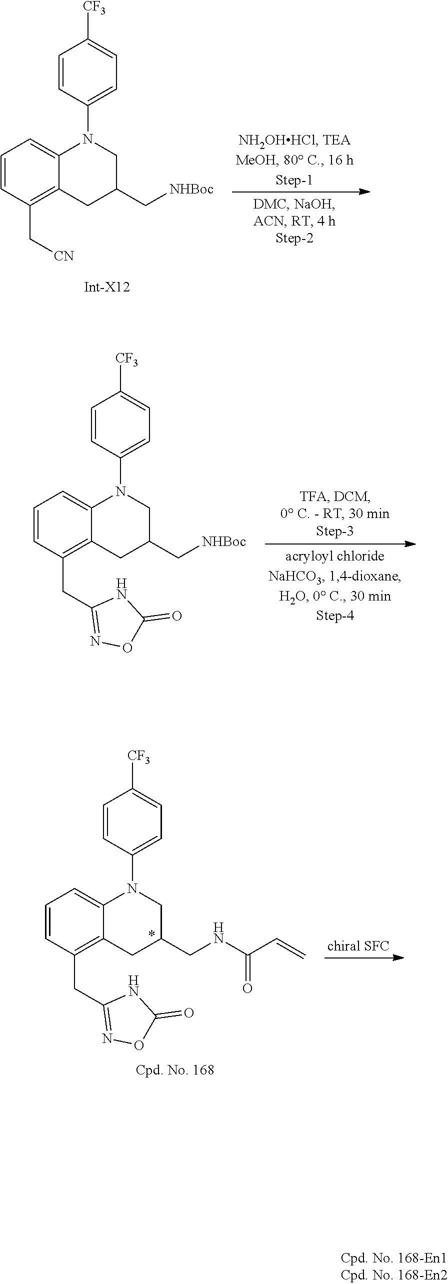 [SMILES: CC(C)(C)OC(=O)NCC1Cc2c(CC#N)cccc2N(c2ccc(C(F)(F)F)cc2)C1 ; "NH2OH·HCl, TEAMeOH, 80° C., 16 hStep-1" ; CC(C)(C)OC(=O)NCC1Cc2c(Cc3noc(=O)[nH]3)cccc2N(c2ccc(C(F)(F)F)cc2)C1 ; "TFA, DCM,0° C. - RT, 30 minStep-3" ; C=CC(=O)NCC1Cc2c(Cc3noc(=O)[nH]3)cccc2N(c2ccc(C(F)(F)F)cc2)C1]

Steps 1-4: These steps were executed in a manner similar (use of appropriate reagents and purification methods known to the person skilled in the art) to Cpd. No. 166. Int-X12 (800 mg, 1.796 mmol, LC/MS 91%) yielded a pale brown gum (500 mg, LC/MS 53%) which was purified by normal phase column chromatography using silica (100-200 mesh) and a gradient of 5% MeOH in DCM as an eluent to afford a pale yellow solid (170 mg, LC/MS 81%). The product was further purified by preparative HPLC method H11. The collected fractions were concentrated and lyophilized to afford N-((5-((5-oxo-4,5-dihydro-1,2,4-oxadiazol-3-yl)methyl)-1-(4-(trifluoromethyl)phenyl)-1,2,3,4-tetrahydroquinolin-3-yl)methyl)acrylamide (Cpd. No. 168) as an off-white solid (85 mg, 11%). (LC/MS; m/z 459.3 [M+H]⁺) Chiral SFC purification: 70 mg of Cpd. No. 168 was purified by preparative SFC method K6 to afford Cpd. No. 168-En1 (18 mg) and Cpd. No. 168-En2 (20 mg), both as an off-white solid. (LC/MS; m/z 459.3 [M+H]⁺). The chiral purity of both enantiomers was assessed by analytic SFC method S6: Cpd. No. 168-En1, 99.9% ee; Cpd. No. 168-En2, 99.2% ee.

Compound Cpd. No. 167 was prepared from Int-X13 in a manner similar to Cpd. No. 166 by using appropriate reagents and purification methods known to the person skilled in the art.

### Example 72: Synthesis of 3-(acrylamidomethyl)-1-(4-(trifluoromethyl)phenyl)-1,2,3,4-tetrahydroquinoline-5-carboxylic acid (Cpd. No. 156)

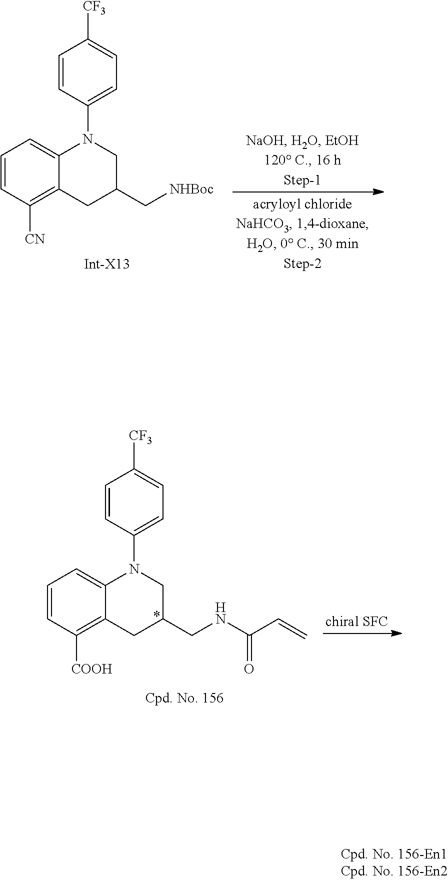 [SMILES: CC(C)(C)OC(=O)NCC1Cc2c(C#N)cccc2N(c2ccc(C(F)(F)F)cc2)C1 ; "NaOH, H2O, EtOH120° C., 16 hStep-1" ; C=CC(=O)NCC1Cc2c(C(=O)O)cccc2N(c2ccc(C(F)(F)F)cc2)C1]

Step 1: A solution of Int-X13 (1.5 g, 3.48 mmol, LC/MS 98%) in EtOH (15 ml) was treated with a 60% aq. NaOH solution (35 mL) and stirred at 120° C. for 16 h. Progress of the reaction was monitored by TLC. TLC mobile phase: 50% EtOAc in pet ether, RF: 0.04, TLC detection: UV. The reaction mixture was cooled to RT and concentrated under reduced pressure to affored sodium 3-(aminomethyl)-1-(4-(trifluoromethyl)phenyl)-1,2,3,4-tetrahydroquinoline-5-carboxylate as a yellow gum (1.0 g, LC/MS 54%) which was taken forward in the subsequent reaction step without further purification. (LC/MS; m/z 351.1 [M+H]⁺)

Step 2: This step was executed in a manner similar (use of appropriate reagents and purification methods known to the person skilled in the art) to step 4 towards Cpd. No. 001. Sodium 3-(aminomethyl)-1-(4-(trifluoromethyl)phenyl)-1,2,3,4-tetrahydroquinoline-5-carboxylate (500 mg, 1.34 mmol, LC/MS 54%) yielded a brown gum (550 mg, LC/MS 54%) which was purified by normal phase chromatography (Combi) using a 40 g reveleris column and a gradient of 8% MeOH in DCM to afford an off-white solid (210 mg, LC/MS 79%). The product was further purified by preparative HPLC method H12. The collected fractions were concentrated under reduced pressure and lyophilised to afford 3-(acrylamidomethyl)-1-(4-(trifluoromethyl)phenyl)-1,2,3,4-tetrahydroquinoline-5-carboxylic acid (Cpd. No. 156) as an off-white solid (83 mg, 28%). (LC/MS; m/z 405.3 [M+H]⁺) Chiral SFC purification: 83 mg of Cpd. No. 156 was purified by preparative SFC method K5 to afford Cpd. No. 156-En1 (17 mg) and Cpd. No. 156-En2 (21 mg), both as an off-white solid. (LC/MS; m/z 405.3 [M+H]⁺) The chiral purity of both enantiomers was assessed by analytic SFC method S6: Cpd. No. 156-En1, 98.7% ee; Cpd. No. 156-En2, 97.8% ee.

Compound Cpd. No. 155 was prepared from Int-X13 in a manner similar to Cpd. No. 156 by using appropriate reagents and purification methods known to the person skilled in the art.

### Example 73: Synthesis of 3-(acrylamidomethyl)—N-(cyclopropylsulfonyl)-1-(4-(trifluoromethyl)phenyl)-1,2,3,4-tetrahydroquinoline-5-carboxamide (Cpd. No. 158)

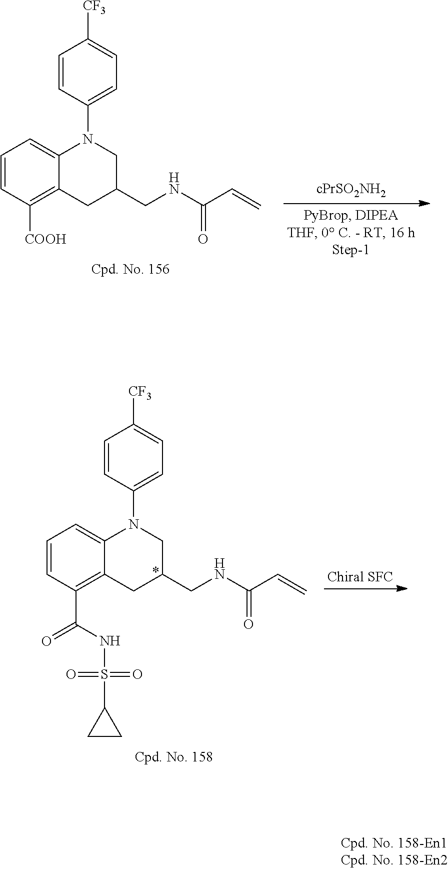 [SMILES: C=CC(=O)NCC1Cc2c(C(=O)O)cccc2N(c2ccc(C(F)(F)F)cc2)C1 ; C=CC(=O)NCC1Cc2c(C(=O)NS(=O)(=O)C3CC3)cccc2N(c2ccc(C(F)(F)F)cc2)C1]

Step 1: This step was executed in a manner similar (use of appropriate reagents and purification methods known to the person skilled in the art) to Cpd. No. 160. Cpd. No. 156 (400 mg, 0.99 mmol, LC/MS 79%) yielded a brown gum (420 mg, LC/MS 32%) which was purified by column chromatography using silica (230-400 mesh) and a gradient of 5% MeOH in DCM to afford a brown solid (350 mg, LC/MS 38%). The product was further purified by preparative HPLC method H13. The collected fractions were concentrated under reduced pressure and lyophilised to afford 3-(acrylamidomethyl)—N-(cyclopropylsulfonyl)-1-(4-(trifluoromethyl)-phenyl)-1,2,3,4-tetrahydroquinoline-5-carboxamide (Cpd. No. 158) as an off-white solid (60 mg, 15%). (LC/MS; m/z 508.3 [M+H]⁺) Chiral SFC purification: 42 mg of Cpd. No. 158 was purified by preparative SFC method K7 to afford Cpd. No. 158-En1 (8 mg) and Cpd. No. 158-En2 (8 mg), both as an off-white solid. (LC/MS; m/z 508.3 [M+H]⁺) The chiral purity of both enantiomers was assessed by analytic SFC method S7: Cpd. No. 158-En1, 99.9% ee; Cpd. No. 158-En2, 97.9% ee.

Compounds Cpd. No. 157 and Cpd. No. 157-En1 (91.3% ee) were prepared from Cpd. No. 156 in a manner similar to Cpd. No. 158 by using appropriate reagents and purification methods known to the person skilled in the art.

### Examples 74-75: Synthesis of N-(1-(cyclohexanecarbonyl)-1,2,3,4-tetrahydroquinolin-3-yl)acrylamide (Cpd. No. 173) and N-(1-(cyclohexylmethyl)-1,2,3,4-tetrahydroquinolin-3-yl)acrylamide (Cpd. No. 175)

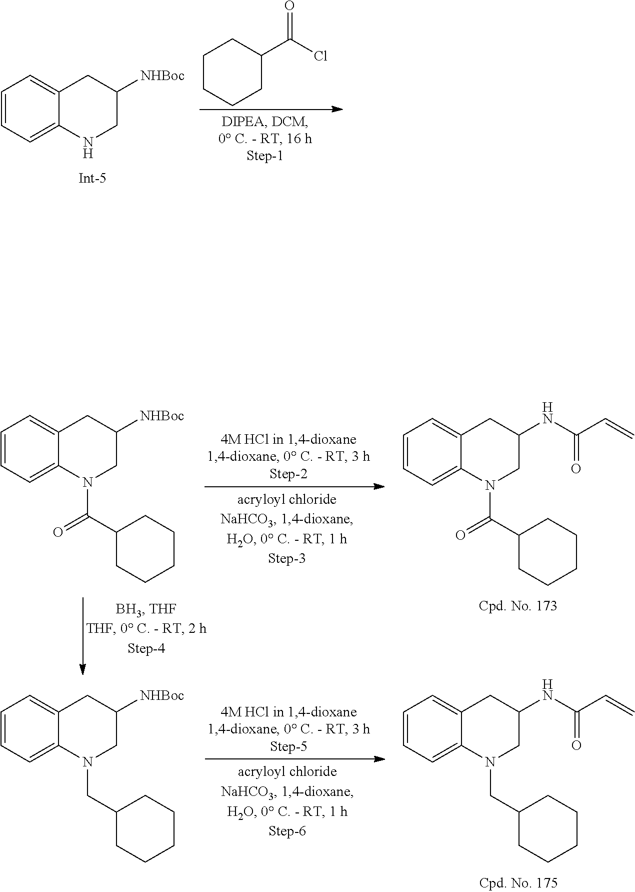 [SMILES: CC(C)(C)OC(=O)NC1CNc2ccccc2C1 ; O=C(Cl)C1CCCCC1 ; CC(C)(C)OC(=O)NC1Cc2ccccc2N(C(=O)C2CCCCC2)C1 ; C=CC(=O)NC1Cc2ccccc2N(C(=O)C2CCCCC2)C1 ; CC(C)(C)OC(=O)NC1Cc2ccccc2N(CC2CCCCC2)C1 ; C=CC(=O)NC1Cc2ccccc2N(CC2CCCCC2)C1]

Step 1: A solution of Int-5 (100 mg, 0.402 mmol, LC/MS 88%) in DCM (5 mL) was cooled to 0° C. and treated with DIPEA (64.6 mg, 0.442 mmol) and cyclohexanecarbonyl chloride (64.6 mg, 0.442 mmol). The reaction mixture was stirred at RT for 16 h. Progress of the reaction was monitored by TLC. TLC mobile phase: 20% EtOAc in pet ether, RF: 0.3, TLC detection: UV. The reaction mixture was diluted with DCM (20 mL), washed with water (2×20 mL) and brine (20 mL). The organic layer was dried over Na₂SO₄ and concentrated to afford tert-butyl (1-(cyclohexanecarbonyl)-1,2,3,4-tetrahydroquinolin-3-yl)carbamate as a pale brown gum (130 mg, 93%, LC/MS 91%) which was taken forward in subsequent reaction without further purification. (LC/MS; m/z 359.4 [M+H]⁺)

Steps 2-3: These steps were executed in a manner similar (use of appropriate reagents and purification methods known to the person skilled in the art) to Cpd. No. 001. Starting material (130 mg, 0.362 mmol, LC/MS 91%) yielded crude product (150 mg, LC/MS 52%), which was purified by preparative HPLC method H2. The collected fractions were concentrated and lyophilised under vacuum to afford N-(1-(cyclohexanecarbonyl)-1,2,3,4-tetrahydroquinolin-3-yl)acrylamide (Cpd. No. 173) as an off-white solid (31 mg, 30%). (LC/MS; m/z 313.3 [M+H]⁺)

Step 4: A solution of tert-butyl (1-(cyclohexanecarbonyl)-1,2,3,4-tetrahydroquinolin-3-yl)carbamate (300 mg, 0.837 mmol, LC/MS 89%) in THF (5 mL) was treated with BH₃·THF (4.18 mL) at 0° C. and stirred at RT for 2 h under a nitrogen atmosphere. Progress of the reaction was monitored by TLC. TLC mobile phase: 20% EtOAc in pet ether, RF: 0.5, TLC detection: UV. The reaction mixture was quenched with MeOH (25 mL) at 0° C. and concentrated under reduced pressure. The obtained residue was dissolved in EtOAc (30 mL), washed with water (30 mL) and brine (30 mL). The organic layer was dried over Na₂SO₄ and concentrated to afford a pale brown gum (250 mg, LC/MS 77%) which was purified by normal phase chromatography using silica gel (100-200 mesh) and a gradient of 11% EtOAc in pet ether as an eluent to afford tert-butyl (1-(cyclohexylmethyl)-1,2,3,4-tetrahydroquinolin-3-yl)carbamate as an off-white gum (200 mg, 74%, LC/MS 95%). (LC/MS; m/z 345.1 [M+H]⁺)

Steps 5-6: These steps were executed in a manner similar (use of appropriate reagents and purification methods known to the person skilled in the art) to Cpd. No. 001. Starting material (200 mg, 0.581 mmol, LC/MS 95%) yielded crude product (160 mg, LC/MS 80%), which was purified by preparative HPLC method H4. The collected fractions were concentrated and lyophilised under vacuum to afford N-(1-(cyclohexylmethyl)-1,2,3,4-tetrahydroquinolin-3-yl)acrylamide (Cpd. No. 175) as an off-white solid (34 mg, 21%). (LC/MS; m/z 299.2 [M+H]⁺)

The following compounds were prepared in a manner similar to Cpd. No. 173 by using appropriate reagents and purification methods known to the person skilled in the art: Cpd. No. 172, Cpd. No. 186 (employing 3-(trifluoromethyl)benzenesulfonyl chloride and K₂CO₃ in ACN at step 1), and Cpd. No. 187 (employing 4-(trifluoromethyl)benzenesulfonyl chloride and K₂CO₃ in ACN at step 1).

From Int-9, the following compounds were prepared in a manner similar to Cpd. No. 173 by using appropriate reagents and purification methods known to the person skilled in the art: Cpd. No. 177, and Cpd. No. 180.

From Int-9, compound Cpd. No. 179 was prepared in a manner similar to Cpd. No. 175 by using appropriate reagents and purification methods known to the person skilled in the art.

### Example 76: Synthesis of N-(1-benzyl-1,2,3,4-tetrahydroquinolin-3-yl)acrylamide (Cpd. No. 174)

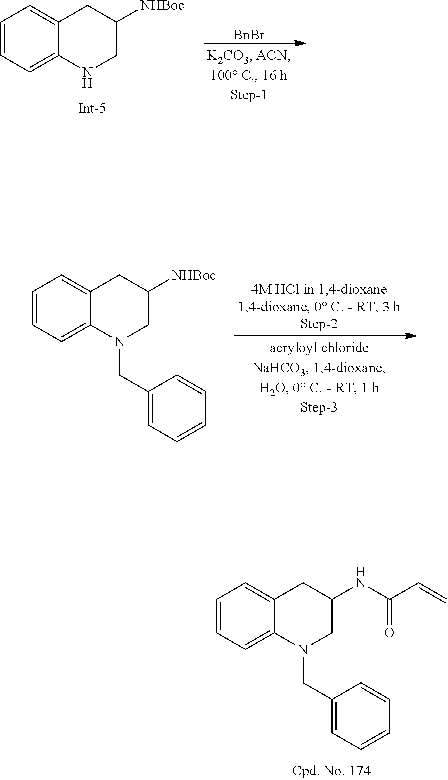

Step 1: A mixture of Int-5 (250 mg, 1.01 mmol, LC/MS 83%), K₂CO₃ (417 mg, 3.02 mmol) and benzyl bromide (206 mg, 1.21 mmol) in ACN (40 mL) was heated at 100° C. for 16 h. Progress of the reaction was monitored by TLC. TLC mobile phase: 20% EtOAc in pet ether, RF: 0.58, TLC detection: UV. The reaction mixture was diluted with water (20 mL) and extracted with EtOAc (2×50 mL). The organic layer was dried over Na₂SO₄ and evaporated under reduced pressure to afford a pale brown gum (350 mg, LC/MS 62%), which was purified by column chromatography using silica gel (100-200 mesh) and 7% EtOAc in pet ether as an eluent to afford tert-butyl (1-benzyl-1,2,3,4-tetrahydroquinolin-3-yl)carbamate as an off-white solid (200 mg, 71%, LC/MS 99%). (LC/MS; m/z 339.3 [M+H]⁺)

Steps 2-3: These steps were executed in a manner similar (use of appropriate reagents and purification methods known to the person skilled in the art) to Cpd. No. 001. Starting material (200 mg, 0.591 mmol, LC/MS 99%) yielded crude product (190 mg, LC/MS 72%), which was purified by preparative HPLC method H6. The collected fractions were concentrated and lyophilised under vacuum to afford N-(1-benzyl-1,2,3,4-tetrahydroquinolin-3-yl)acrylamide (Cpd. No. 174) as an off-white solid (30 mg, 17%). (LC/MS; m/z 293.2 [M+H]⁺)

The following compounds were prepared in a manner similar to Cpd. No. 174 by using appropriate reagents and purification methods known to the person skilled in the art: Cpd. No. 181, Cpd. No. 182, and Cpd. No. 191.

From Int-9, the following compounds were prepared in a manner similar to Cpd. No. 174 by using appropriate reagents and purification methods known to the person skilled in the art: Cpd. No. 176, Cpd. No. 184, and Cpd. No. 185.

### Example 77: Synthesis of N-((1-(4-(trifluoromethyl)phenyl)-1,2,3,4-tetrahydroquinolin-2-yl)methyl)acrylamide (Cpd. No. 178)

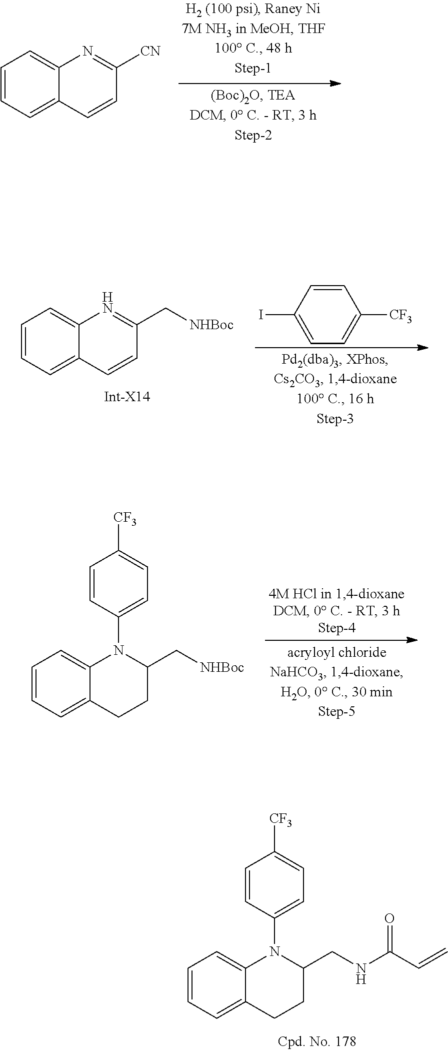

Steps 1-2: These steps were executed in a manner similar (use of appropriate reagents and purification methods known to the person skilled in the art) to steps 1-2 towards Cpd. No. 084. Quinoline-2-carbonitrile (2.0 g, 13.0 mmol) yielded tert-butyl ((1,2,3,4-tetrahydroquinolin-2-yl)methyl)carbamate (Int-X14) as an off-white solid (1.8 g, 50%, LC/MS 94%). (LC/MS; m/z 263.1 [M+H]⁺)

Step 3: A solution of Int-X14 (500 mg, 1.90 mmol, LC/MS 94%), 1-iodo-4-(trifluoromethyl)benzene (1.03 g, 3.81 mmol) and Cs₂CO₃ (1.24 g, 3.81 mmol) in 1,4-dioxane (10 mL) was degassed with argon for 5 min. To the solution was added Pd₂(dba)₃ (175 mg, 0.19 mmol) and XPhos (179 mg. 0.38 mmol) and the reaction mixture was stirred at 110° C. for 16 h (sealed tube). Progress of the reaction was monitored by TLC. TLC mobile phase: 10% EtOAc in pet ether, RF: 0.32, TLC detection: UV. The reaction mixture was concentrated under reduced pressure to afford crude product (700 mg, LC/MS 26%), which was purified by normal phase chromatography (Grace) using a 24 g reveleris column and a gradient of 8% EtOAc in pet ether as an eluent to afford tert-butyl ((1-(4-(trifluoromethyl)phenyl)-1,2,3,4-tetrahydroquinolin-2-yl)methyl)carbamate as a pale yellow gum (300 mg, 28%, LC/MS 68%). (LC/MS; m/z 407.3 [M+H]⁺)

Steps 4-5: These steps were executed in a manner similar (use of appropriate reagents and purification methods known to the person skilled in the art) to steps 4-5 towards Cpd. No. 084. Starting material (300 mg, 0.739 mmol, LC/MS 68%) yielded crude product (250 mg), which was purified by preparative HPLC method H7. The collected fractions were concentrated and lyophilised under vacuum to afford N-((1-(4-(trifluoromethyl)phenyl)-1,2,3,4-tetrahydroquinolin-2-yl)methyl)acrylamide (Cpd. No. 178) as an off-white solid (57 mg, 31%). (LC/MS; m/z 361.2 [M+H]⁺)

Compound Cpd. No. 188 was prepared in a manner similar to Cpd. No. 178 by using appropriate reagents and purification methods known to the person skilled in the art.

From Int-X14, the following compounds were prepared in a manner similar to Cpd. No. 174 by using appropriate reagents and purification methods known to the person skilled in the art: Cpd. No. 189 and Cpd. No. 190.

### Example 78: Synthesis of N-(1-(1-(4-(trifluoromethyl)phenyl)-1,2,3,4-tetrahydroquinolin-3-yl)ethyl)acrylamide (Cpd. No. 192)

### Synthesis of 6-bromo-1-methyl-1H-pyrazolo[4,3-b]pyridine (Int-X23) and 6-bromo-2-methyl-2H-pyrazolo[4,3-b]pyridine (Int-X24)

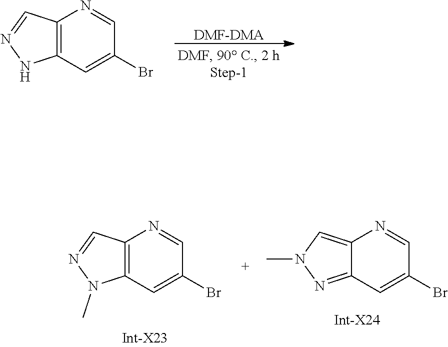

Step 1: To a solution of DMF-DMA (2.7 mL, 20.2 mmol) in dry DMF (10 mL) was added 6-bromo-1H-pyrazolo[4,3-b]pyridine (1.0 g, 5.05 mmol). The reaction mixture was stirred at 90° C. for 2 h (sealed tube). The reaction mixture was diluted with water (20 mL) and extracted with EtOAc (2×50 mL). The organic layer was washed with brine (50 mL), dried over Na₂SO₄ and concentrated under reduced pressure. The residue was purified by flash chromatography using a 40 g ecoflex column and a gradient of 10-80% EtOAc in pet ether as an eluent to afford 6-bromo-1-methyl-1H-pyrazolo[4,3-b]pyridine (Int-X23) (642 mg, 60%) and 6-bromo-2-methyl-2H-pyrazolo[4,3-b]pyridine (Int-X24) (312 mg, 29%), both as an off-white solid. (LC/MS; m/z 212.2 [M+H]⁺). Int-X23: ¹H NMR (400 MHz, DMSO-d₆) b ppm: 8.58 (s, 2H), 8.31 (s, 1H), 4.07 (s, 3H); Int-X24: ¹H NMR (400 MHz, DMSO-d₆) b ppm: 8.72 (s, 1H), 8.54 (d, 1H), 8.43 (dd, 1H), 4.22 (s, 3H).

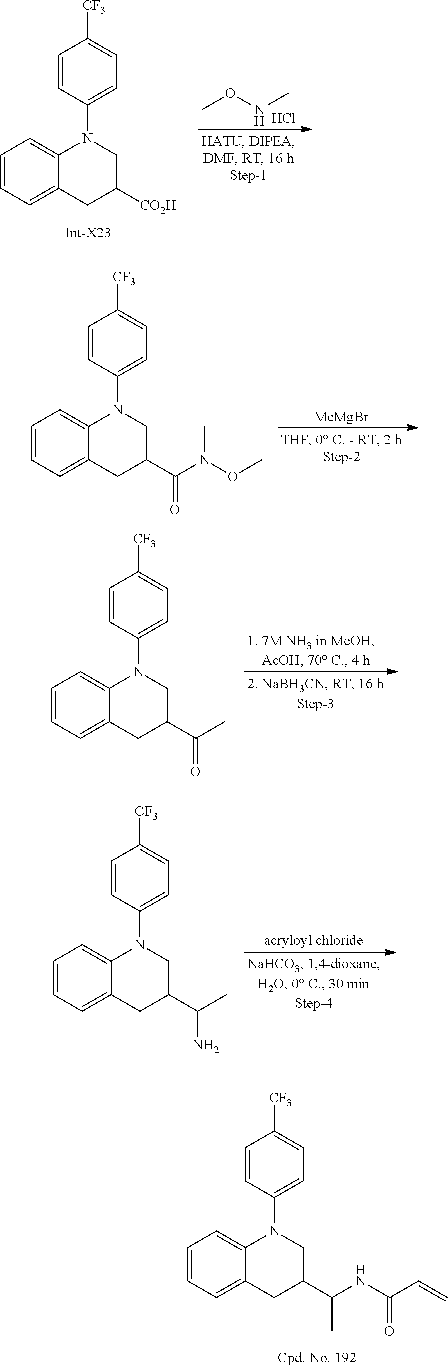

Step 1: A solution of Int-X23 (2.8 g, 8.74 mmol, LC/MS 68%) in DMF (30 mL) was cooled to 0° C. and treated with N,O-dimethyl hydroxylamine hydrochloride (1.28 g, 13.11 mmol), HATU (4.98 g, 13.1 mmol) and DIPEA (3.4 g, 26.22 mmol) under a nitrogen atmosphere. The reaction mixture was stirred at RT for 16 h. Progress of the reaction was monitored by TLC. TLC mobile phase: 50% EtOAc in pet ether, RF: 0.53, TLC detection: UV. The reaction mixture was diluted with ice water (100 mL) and extracted with EtOAc (2×100 mL). The organic layer was dried over Na₂SO₄ and concentrated under reduced pressure to afford a pale brown oil (3.0 g). The crude product was purified by normal phase chromatography (Combi) using a 24 g reveleris column and a gradient of 32% EtOAc in pet ether as an eluent to afford N-methoxy-N-methyl-1-(4-(trifluoromethyl)phenyl)-1,2,3,4-tetrahydroquinoline-3-carboxamide as a pale yellow gum (2.0 g, 93%, LC/MS 99%). (LC/MS; m/z 365.3 [M+H]⁺)

Step 2: A solution of N-methoxy-N-methyl-1-(4-(trifluoromethyl)phenyl)-1,2,3,4-tetrahydroquinoline-3-carboxamide (2.0 g, 5.49 mmol, LC/MS 99%) in THF (20 mL) was treated with methylmagnesium bromide (3M in Et₂O; 2.0 mL, 6.04 mmol) at 0° C. The reaction mixture was stirred at RT for 2 h. Progress of the reaction was monitored by TLC. TLC mobile phase: 70% EtOAc in pet ether, RF: 0.63, TLC detection: UV. The reaction mixture was quenched with aqueous NH₄Cl (20 mL) and extracted with EtOAc (2×50 mL). The organic layer was dried over Na₂SO₄ and concentrated under reduced pressure to afford a pale yellow solid (1.8 g). The crude product was purified by normal phase chromatography (Combi) using a 24 g reveleris column and a gradient of 35% EtOAc in pet ether as an eluent to afford 1-(1-(4-(trifluoromethyl)phenyl)-1,2,3,4-tetrahydroquinolin-3-yl)ethan-1-one as a pale yellow solid (1.3 g, 72%, LC/MS 96%). (LC/MS; m/z 320.2 [M+H]⁺)

Step 3: A solution of 1-(1-(4-(trifluoromethyl)phenyl)-1,2,3,4-tetrahydroquinolin-3-yl)ethan-1-one (500 mg, 1.56 mmol, LC/MS 96%) in 7M NH₃ in MeOH (6.0 mL) was treated with AcOH (0.6 mL) and stirred at 70° C. for 4 h (sealed tube). The reaction mixture was cooled to RT, treated with NaBH₃CN (197 mg, 3.13 mmol) and stirred at RT for 16 h. Progress of the reaction was monitored by TLC. TLC mobile phase: 10% MeOH in DCM, RF: 0.43, TLC detection: UV. The reaction mixture was concentrated under reduced pressure, dissolved in 10% MeOH in DCM (50 mL) and washed with brine (10 mL). The organic layer was dried over Na₂SO₄ and concentrated under reduced pressure to afford a pale brown solid (380 mg, 49%, LC/MS 62%; mixture of diastereoisomers). (LC/MS; m/z 321.3 [M+H]⁺). The product was used without further purification in the next step.

Step 4: This step was executed in a manner similar (use of appropriate reagents and purification methods known to the person skilled in the art) to step 5 towards Cpd. No. 084. Starting material (380 mg, 1.186 mmol, LC/MS 62%) yielded crude product (450 mg, LC/MS 48%), which was purified by preparative HPLC method H3. The collected fractions were concentrated and lyophilised under vacuum to afford N-(1-(1-(4-(trifluoromethyl)phenyl)-1,2,3,4-tetrahydroquinolin-3-yl)ethyl)acrylamide (Cpd. No. 192) as an off-white solid (89 mg, 32%; 35:65 mixture of diastereoisomers). (LC/MS; m/z 375.2 [M+H]⁺)

### Example 79: Synthesis of (E)-4-acetamido-N-((1-(4-(trifluoromethyl)phenyl)-1,2,3,4-tetrahydroquinolin-3-yl)methyl)but-2-enamide (Cpd. No. 193)

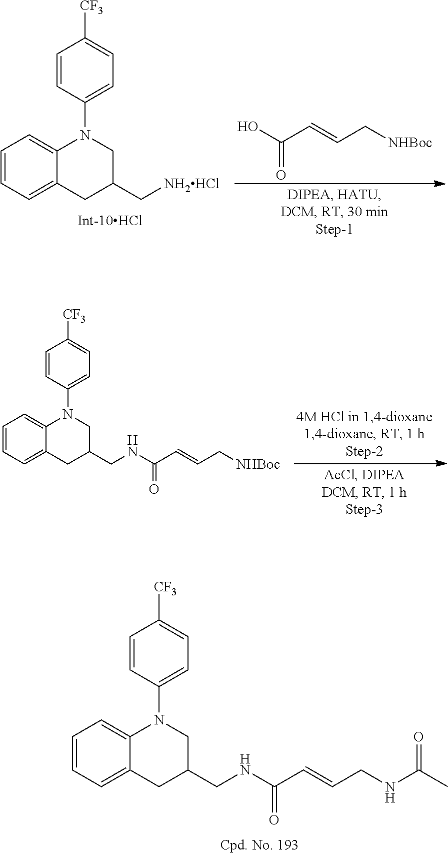

Step 1: To a solution of (E)-4-(tert-butoxycarbonylamino)but-2-enoic acid (32 mg, 0.16 mmol), DIPEA (0.1 mL, 0.58 mmol) and HATU (68 mg, 0.18 mmol) in DCM (1.0 mL) was added Int-10·HCl (50 mg, 0.15 mmol). The reaction mixture was stirred at RT for 30 min.

The mixture was diluted with DCM (20 mL), washed sequentially with water (10 mL), a 0.1M HCl solution (10 mL) and a saturated NaHCO₃ solution (10 mL). The organic layer was dried over Na₂SO₄, filtered and concentrated. The residue was purified by column chromatography using a 12 g ecoflex column and a gradient of EtOAc in pet ether (20% to 100%) as an eluent to afford tert-butyl (E)-(4-oxo-4-(((1-(4-(trifluoromethyl)phenyl)-1,2,3,4-tetrahydroquinolin-3-yl)methyl)amino)but-2-en-1-yl)carbamate (40 mg, 56%). (LC/MS; m/z 490.0 [M+H]⁺)

Step 2: To a mixture of tert-butyl (E)-(4-oxo-4-(((1-(4-(trifluoromethyl)phenyl)-1,2,3,4-tetrahydroquinolin-3-yl)methyl)amino)but-2-en-1-yl)carbamate (40 mg, 0.08 mmol) in 1,4-dioxane (0.4 mL) was added a solution of 4M HCl (0.4 mL, 1.6 mmol) in 1,4-dioxane. The reaction mixture was stirred at RT for 1h. The mixture was concentrated to yield crude (E)-4-amino-N-((1-(4-(trifluoromethyl)phenyl)-1,2,3,4-tetrahydroquinolin-3-yl)methyl)but-2-enamide hydrochloride (32 mg, 92% yield). (LC/MS; m/z 390.0 [M+H]⁺). The product was used without further purification in the next step.

Step 3: To a suspension of acetyl chloride (3.85 μL, 0.05 mmol) and (E)-4-amino-N-((1-(4-(trifluoromethyl)phenyl)-1,2,3,4-tetrahydroquinolin-3-yl)methyl)but-2-enamide hydrochloride (15 mg, 0.04 mmol) in DCM (0.4 mL) was added DIPEA (24 μL, 0.14 mmol) and the suspension was stirred at RT for 1h. The mixture was concentrated and the residue was purified by column chromatography using a 12 g reveleris column and a gradient of 15% MeOH in DCM to afford (E)-4-acetamido-N-((1-(4-(trifluoromethyl)phenyl)-1,2,3,4-tetrahydroquinolin-3-yl)methyl)but-2-enamide (Cpd. No. 193) as an off-wite solid (6 mg, 39%). (LC/MS; m/z 431.0 [M+H]⁺)

### Example 80: Synthesis of 3-bromo-5-(1-(4-(trifluoromethyl)phenyl)-1,2,3,4-tetrahydroquinolin-3-yl)-4,5-dihydroisoxazole (Cpd. No. 194)

### Synthesis of ethyl 1-(4-(trifluoromethyl)phenyl)-1,2,3,4-tetrahydroquinoline-3-carboxylate (Int-X22)

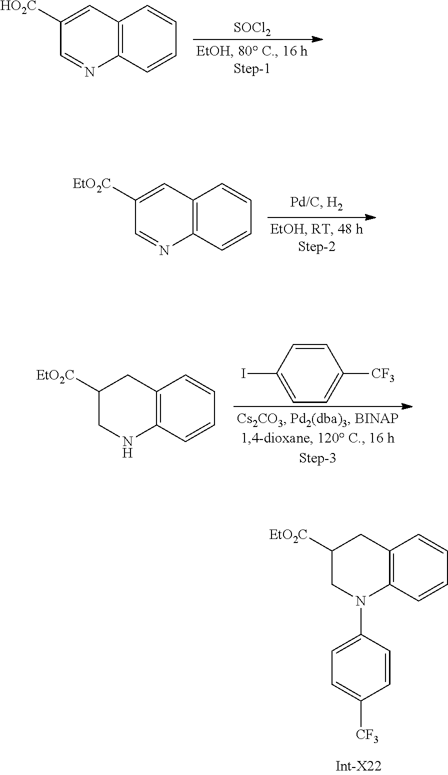

Step 1: A solution of quinoline-3-carboxylic acid (25.0 g, 144 mmol) in EtOH (500 mL) was treated with thionyl chloride (171 g, 1.44 mol) at 0° C. The reaction mixture was stirred at 80° C. for 16 h. Progress of the reaction was monitored by TLC. TLC mobile phase: 50% EtOAc in pet ether, RF: 0.28, TLC detection: UV. The reaction mixture was cooled to RT and concentrated under reduced pressure to afford an off-white solid which was suspended in Et₂O (500 ml). The suspension was stirred at RT for 1 h, filtered, washed with Et₂O (200 mL) and dried to afford ethyl quinoline-3-carboxylate as an off-white solid (25 g, 86%, LC/MS 97%). (LC/MS; m/z 202.1 [M+H]⁺)

Step 2: To a solution of ethyl quinoline-3-carboxylate (25 g, 124 mmol) in EtOH (500 mL) was added 10% Pd/C (25 g) under a nitrogen atmosphere. The mixture was stirred at RT for 48 h under a hydrogen atmosphere (balloon pressure). Progress of the reaction was monitored by TLC. TLC mobile phase: 50% EtOAc in pet ether, RF: 0.15, TLC detection: UV. The reaction mixture was filtered through a celite pad and washed with EtOH (250 mL). The filtrate was concentrated under reduced pressure to afford a brown gum (22 g), which was purified by column chromatography using silica gel (230-400 mesh) and a gradient of 30% of EtOAc in pet ether as an eluent to afford ethyl 1,2,3,4-tetrahydroquinoline-3-carboxylatecrude as a colourless gum (20 g, 73%, LC/MS 91%). (LC/MS; m/z 206.1 [M+H]⁺)

Step 3: A solution of ethyl 1,2,3,4-tetrahydroquinoline-3-carboxylate (5 g, 24.4 mmol, LC/MS 91%) in 1,4-dioxane (35 ml) was treated with Cs₂CO₃ (19.8 g, 60.9 mmol), 1-iodo-4-(trifluoromethyl)benzene (9.94 g, 36.5 mmol) and degassed with argon for 5 min. To the solution as added BINAP (1.95 g, 2.92 mmol) and Pd₂(dba)₃ (1.34 g, 1.46 mmol). The reaction mixture was stirred at 120° C. for 16 h (sealed tube). Progress of the reaction was monitored by TLC. TLC mobile phase: 10% EtOAc in pet ether, RF: 0.37, TLC detection: UV. The reaction mixture was cooled to RT, diluted with EtOAc (20 mL), filtered through a celite pad which was washed with EtOAc (50 mL). The filtrate was concentrated under reduced pressure to afford a pale yellow gum (5.2 g). The crude product was purified by normal phase chromatography (Combi) using silica gel (230-400 mesh) and a gradient of 5% EtOAc in pet ether to afford ethyl 1-(4-(trifluoromethyl)phenyl)-1,2,3,4-tetrahydroquinoline-3-carboxylate (Int-X22) as a pale yellow gum (3.1 g, 32%, LC/MS 81%). (LC/MS; m/z 350.3 [M+H]⁺)

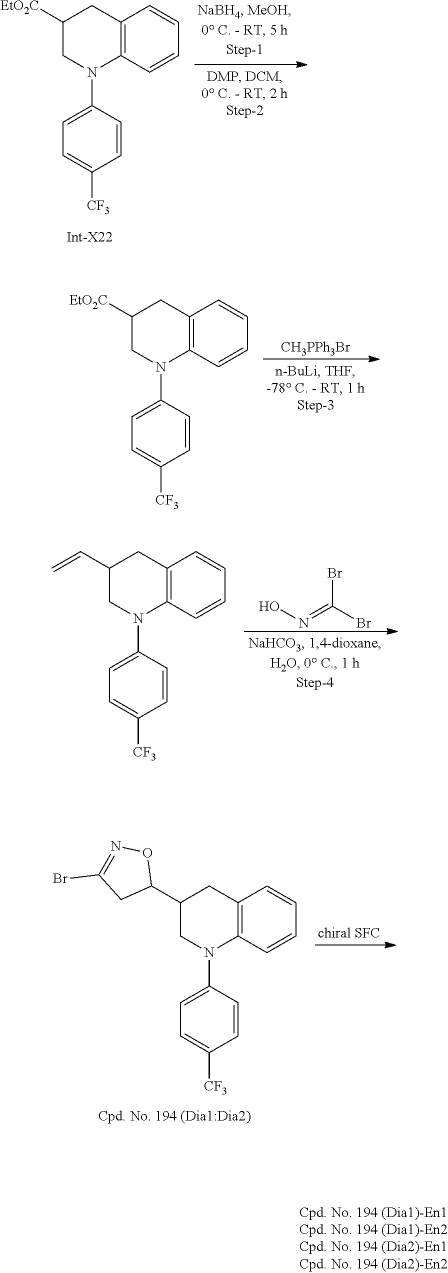 [SMILES: CCOC(=O)C1Cc2ccccc2N(c2ccc(C(F)(F)F)cc2)C1 ; CCOC(=O)C1Cc2ccccc2N(c2ccc(C(F)(F)F)cc2)C1 ; C=CC1Cc2ccccc2N(c2ccc(C(F)(F)F)cc2)C1 ; ON=C(Br)Br ; FC(F)(F)c1ccc(N2CC(C3CC(Br)=NO3)Cc3ccccc32)cc1 ; "CH3PPh3Br"]

Step 1: A solution of Int-X22 (9.5 g, 27.2 mmol) in MeOH (50 mL) was cooled to 0° C., treated with NaBH₄ (5.19 g, 136 mmol) and stirred at RT for 4 h. Progress of the reaction was monitored by TLC. TLC mobile phase: 50% EtOAc in pet ether, RF: 0.24, TLC detection: UV. The reaction mixture was quenched with ice water (25 mL) and partially concentrated under reduced pressure. The aqueous layer was extracted with EtOAc (3×50 mL), washed with brine (100 mL), dried over Na₂SO₄ and concentrated under reduced pressure to afford a brown gum (10 g, LC/MS 92%). The crude product was purified by normal phase chromatography using silica gel (230-400 mesh) and a gradient of 30% EtOAc in pet ether as an eluent to afford (1-(4-(trifluoromethyl)phenyl)-1,2,3,4-tetrahydroquinolin-3-yl)methanol as a pale yellow solid (5 g, 59%, LC/MS 98%). (LC/MS; m/z 308.3 [M+H]⁺)

Step 2: A solution of (1-(4-(trifluoromethyl)phenyl)-1,2,3,4-tetrahydroquinolin-3-yl)methanol (2.5 g, 8.13 mmol, LC/MS 98%) in DCM (50 ml) was cooled to 0° C. and treated with Dess-martin periodinane (6.9 g, 16.27 mmol). The reaction mixture was stirred at RT for 2 h. Progress of the reaction was monitored by TLC. TLC mobile phase: 30% EtOAc in pet ether, RF: 0.31, TLC detection: UV. The reaction mixture was diluted with DCM (50 mL) and filtered through a celite pad. The filtrate was washed with aqueous NaHCO₃ and extracted with DCM (2×25 mL). The organic layer was washed with brine (100 mL), dried over Na₂SO₄ and concentrated under reduced pressure to afford a brown gum (2 g) which was used in the next step without further purification.

Step 3: A solution of methyltriphenylphosphonium bromide (2.80 g, 7.86 mmol) in THF (20 ml) was cooled to −78° C., treated with n-BuLi (2.5M in hexanes, 5.20 mL, 13.10 mmol) and stirred at −78° C. for 45 min. The reaction mixture was treated with a solution of 1-(4-(trifluoromethyl) phenyl)-1,2,3,4-tetrahydroquinoline-3-carbaldehyde (2.0 g, 6.55 mmol, crude) in THF (5.0 mL) and stirred at RT for 16 h. Progress of the reaction was monitored by TLC. TLC mobile phase: 5% EtOAc in pet ether, RF: 0.50, TLC detection: UV. The reaction mixture was quenched with aqueous NH₄Cl (20 mL) and extracted with EtOAc (3×25 mL). The organic layer was washed with brine (40 mL), dried over Na₂SO₄ and concentrated under reduced pressure. The crude product was purified by normal phase chromatography using silica gel (230-400 mesh) and pet ether as an eluent to afford 1-(4-(trifluoromethyl)phenyl)-3-vinyl-1,2,3,4-tetrahydroquinoline as a pale yellow solid (1.2 g, 38% (2 steps), LC/MS 77%). (LC/MS; m/z 304.2 [M+H]⁺)

Step 4: A solution of 1-(4-(trifluoromethyl)phenyl)-3-vinyl-1,2,3,4-tetrahydroquinoline (1.5 g, 4.94 mmol) in 1,4-dioxane (15 ml) and water (3.0 ml) was cooled to 0° C. and treated with NaHCO₃ (1.03 g, 12.37 mmol) and hydroxycarbonimidic dibromide (1.20 g, 5.93 mmol). The reaction mixture was stirred at 0° C. for 1 h. Progress of the reaction was monitored by TLC. TLC mobile phase: 40% EtOAc in pet ether, RF: 0.16, TLC detection: UV. The reaction mixture was diluted with ice water (20 mL) and extracted with EtOAc (3×50 mL). The organic layer was washed with brine (50 mL), dried over Na₂SO₄ and concentrated under reduced pressure to afford a brown gum (1.8 g, LC/MS 72%). The crude product was purified by normal phase chromatography using silica gel (230-400 mesh) and a gradient of 35% EtOAc in pet ether as an eluent to afford a pale yellow gum (800 mg, LC/MS 79%). The product was further purified by preparative HPLC method H11. The collected fractions were concentrated and lyophilised to afford 3-bromo-5-(1-(4-(trifluoromethyl)phenyl)-1,2,3,4-tetrahydroquinolin-3-yl)-4,5-dihydroisoxazole (Cpd. No. 194) as an off-white solid (215 mg, 10%). (LC/MS; m/z 425.3 [M+H]⁺). Chiral SFC purification: 200 mg of Cpd. No. 194 was purified by preparative method H16 to afford Cpd. No. 194(Dia1)-En1 (11 mg), Cpd. No. 194(Dia2)-En1 (27 mg), and a mixture of isomers, which was further purified by preparative SFC method K10 to afford Cpd. No. 194(Dia2)-En2 (21 mg) and Cpd. No. 194(Dia1)-En2 (15 mg); the compounds were isolated as an off-white solid. (LC/MS; m/z 425.3 [M+H]⁺). The chiral purity of the enantiomers was assessed by analytic SFC method S2: Cpd. No. 194(Dia1)-En1, 99.5% ee; Cpd. No. 194(Dia2)-En1, 99.3% ee; Cpd. No. 194(Dia2)-En2, 99.8% ee; Cpd. No. 194(Dia1)-En2, 99.8% ee.

TABLE 2
Analytical data for synthesized compounds of the invention

| | [M + H]⁺ | LC/MS | RT | |
|---|---|---|---|---|
| Cpd | (m/z): | Method | (min.) | ¹H NMR (δ ppm) |
| 001 | 347.2 | L2 | 3.04 | (DMSO-d₆) δ ppm: 8.21-8.23 (d, 1H), 7.60-7.62 (d, 2H), 7.34- |
| | | | | 7.36 (d, 2H), 7.16-7.18 (m, 1H), 7.03-7.06 (m, 1H), 6.97-7.00 |
| | | | | (m, 1H), 6.86-6.90 (m, 1H), 6.17-6.23 (m, 1H), 6.01-6.06 (dd, |
| | | | | 1H), 5.53-5.56 (dd, 1H), 4.14-4.22 (m, 1H), 3.83-3.87 (m, 1H), |
| | | | | 3.44-3.49 (m, 1H), 3.06-3.11 (m, 1H), 2.74-2.81 (m, 1H). |
| 001-En1 | 347.2 | S1 | 3.02 | (DMSO-d₆) δ ppm: 8.22-8.23 (d, 1H), 7.60-7.62 (d, 2H), 7.34- |
| | | | | 7.36 (d, 2H), 7.16-7.18 (m, 1H), 7.03-7.07 (m, 1H), 6.97-7.00 |
| | | | | (m, 1H), 6.86-6.90 (m, 1H), 6.17-6.23 (m, 1H), 6.01-6.06 (dd, |
| | | | | 1H), 5.53-5.56 (dd, 1H), 4.14-4.20 (m, 1 H), 3.83-3.87 (m, 1 H), |
| | | | | 3.44-3.49 (m, 1 H), 3.06-3.14 (m, 1 H), 2.74-2.81 (m, 1 H). |
| 001-En2 | 347.2 | S1 | 3.02 | (DMSO-d₆) δ ppm: 8.22-8.23 (d, 1H), 7.60-7.62 (d, 2H), 7.34- |
| | | | | 7.36 (d, 2H), 7.16-7.18 (m, 1H), 7.03-7.07 (m, 1H), 6.97-7.00 |
| | | | | (m, 1H), 6.86-6.90 (m, 1H), 6.17-6.23 (m, 1H), 6.01-6.06 (dd, |
| | | | | 1H), 5.53-5.56 (dd, 1H), 4.14-4.20 (m, 1H), 3.83-3.87 (m, 1H), |
| | | | | 3.44-3.49 (m, 1H), 3.06-3.14 (m, 1H), 2.74-2.81 (m, 1H). |
| 002 | 279.2 | L2 | 2.75 | (DMSO-d₆) δ ppm: 8.20 (d, 1H), 7.34-7.38 (m, 2H), 7.21-7.23 |
| | | | | (m, 2H), 7.09-7.12 (m, 2H), 6.90-6.94 (m, 1H), 6.68-6.72 (m, |
| | | | | 1H), 6.62-6.65 (d, 1H), 6.23-6.30 (m, 1H), 6.04-6.09 (dd, 1H), |
| | | | | 5.55-5.58 (dd, 1H), 4.20-4.24 (m, 1H), 3.70-3.74 (m, 1H), 3.36- |
| | | | | 3.41 (t, 1H), 3.04-3.10 (m, 1H), 2.75-2.81 (m, 1H). |
| 003 | 347.3 | L2 | 2.98 | (DMSO-d₆) δ ppm: 8.17-8.25 (d, 1H), 7.49-7.58 (m, 3H), 7.33- |
| | | | | 7.39 (m, 1H), 7.12-7.18 (m, 1H), 6.97-7.05 (m, 1H), 6.77-6.84 |
| | | | | (m, 2H), 6.17-6.27 (m, 1H), 6.00-6.06 (dd, 1H), 5.51-5.58 (dd, |
| | | | | 1H), 4.16-4.27 (m, 1H), 3.75-3.84 (m, 1H), 3.45-3.54 (m, 1H), |
| | | | | 3.05-3.13 (m, 1H), 2.73-2.82 (m, 1H). |
| 004 | 321.3 | L2 | 3.17 | (DMSO-d₆) δ ppm: 8.14-8.22 (d, 1H), 7.20-7.29 (m, 2H), 7.11- |
| | | | | 7.17 (m, 2H), 7.03-7.08 (m, 1H), 6.86-6.94 (m, 1H), 6.63-6.70 |
| | | | | (m, 1H), 6.55-6.60 (m, 1H), 6.23-6.33 (m, 1H), 6.02-6.11 (dd, |
| | | | | 1H), 5.53-5.59 (dd, 1H), 4.14-4.28 (m, 1H), 3.66-3.71 (m, 1H), |
| | | | | 3.35-3.40 (m, 1H), 3.03-3.09 (m, 1H), 2.74-2.95 (m, 2H), 1.16- |
| | | | | 1.25 (d, 6H). |
| 005 | 321.3 | L2 | 3.14 | (DMSO-d₆) δ ppm: 8.16-8.22 (d, 1H), 7.24-7.30 (m, 1H), 7.10- |
| | | | | 7.13 (m, 1H), 6.99-7.08 (m, 3H), 6.89-6.95 (m, 1H), 6.65-6.72 |
| | | | | (m, 1H), 6.60-6.63 (m, 1H), 6.23-6.32 (m, 1H), 6.04-6.10 (dd, |
| | | | | 1H), 5.54-5.61 (dd, 1H), 4.18-4.28 (m, 1H), 3.68-3.75 (m, 1H), |
| | | | | 3.36-3.44 (m, 1H), 3.03-3.10 (m, 1H), 2.73-2.91 (m, 2H), 1.17- |
| | | | | 1.21 (d, 6H). |
| 006 | 309.2 | L2 | 2.74 | DMSO-d₆) δ ppm: 8.19-8.21 (d, 1H), 7.23-7.27 (t, 1H), 7.06- |
| | | | | 7.08 (d, 1H), 6.921-6.923 (m, 1H), 6.76-6.80 (m, 2H), 6.51-6.73 |
| | | | | (m, 3H), 6.23-6.30 (m, 1H), 6.04-6.09 (dd, 1H), 5.56-5.59 (dd, |
| | | | | 1H), 4.20-4.24 (m, 1H), 3.71-3.74 (m, 4H), 3.35-3.40 (m, 1H), |
| | | | | 3.04-3.09 (m, 1H), 2.74-2.80 (m, 1H). |
| 007 | 309.2 | L4 | 2.73 | (DMSO-d₆) δ ppm: 8.18 (d, 1H), 7.15-7.18 (m, 2H), 6.95-7.02 |
| | | | | (m, 3H), 6.85-6.89 (t, 1H), 6.59-6.63 (t, 1H), 6.26-6.37 (m, 2H), |
| | | | | 6.05-6.10 (dd, 1H), 5.56-5.59 (dd, 1H), 4.23-4.27 (m, 1H), 3.75 |
| | | | | (s, 3H), 3.60-3.63 (dd, 1H), 3.31-3.37 (m, 1H), 3.03-3.08 (dd, |
| | | | | 1H), 2.75-2.81 (m, 1H). |
| 008 | 355.2 | L2 | 3.16 | (DMSO-d₆) δ ppm: 8.23 (d, 1H), 7.63-7.67 (m, 4H), 7.43-7.47 |
| | | | | (t, 2H), 7.29-7.35 (m, 3H), 7.10 (d, 1H), 6.96-7.09 (t, 1H), 6.74- |
| | | | | 6.80 (m, 2H), 6.23-6.30 (m, 1H), 6.04-6.09 (dd, 1H), 5.55-5.58 |
| | | | | (m, 1H), 4.21-4.25 (m, 1H), 3.78-3.81 (dd 1H), 3.31-3.45 (m, |
| | | | | 1H), 3.06-3.12 (dd, 1H), 2.77-2.83 (m, 1H). |
| 009 | 355.3 | L2 | 3.14 | (DMSO-d₆) δ ppm: 8.20-8.25 (d, 1H), 7.62-7.66 (m, 2H), 7.44- |
| | | | | 7.52 (m, 4H), 7.35-7.40 (m, 2H), 7.21-7.25 (m, 1H), 7.08-7.12 |
| | | | | (m, 1H), 6.95-6.99 (m, 1H), 6.72-6.79 (m, 2H), 6.23-6.32 (m, |
| | | | | 1H), 6.04-6.09 (dd, 1H), 5.53-5.59 (dd, 1H), 4.22-4.31 (m, 1H), |
| | | | | 3.78-3.83 (m, 1H), 3.45-3.51 (m, 1H), 3.07-3.13 (m, 1H), 2.76- |
| | | | | 2.85 (m, 1H). |
| 010 | 348.2 | L2 | 2.66 | (DMSO-d₆) δ ppm: 8.601 (s, 1H), 8.246-8.263 (d, 1H), 7.741- |
| | | | | 7.745 (m, 2H), 7.206-7.226 (m, 1H), 7.054-7.094 (m, 2H), |
| | | | | 6.934-6.974 (m, 1H), 6.129-6.197 (m, 1H), 5.979-6.028 (dd, |
| | | | | 1H), 5.503-5.534 (dd, 1H), 4.184-4.225 (m, 1H), 3.829-3.867 |
| | | | | (m, 1H), 3.573-3.621 (m, 1H), 3.092-3.147 (m, 1H), 2.765- |
| | | | | 2.825 (m, 1H). |
| 011 | 348.2 | L2 | 2.74 | (DMSO-d₆) δ ppm: 8.53-8.54 (s, 1H), 8.24-8.26 (d, 1H), 7.84- |
| | | | | 7.87 (d, 1H), 7.36-7.38 (d, 1H), 7.22-7.28 (m, 2H), 7.18-7.20 (t, |
| | | | | 1H), 7.06-7.10 (t, 1H), 6.00-6.16 (m, 2H), 5.51-5.55 (dd, 1H), |
| | | | | 4.14-4.22 (m, 2H), 3.72-3.77 (m, 1H), 3.08-3.13 (dd, 1H), 2.71- |
| | | | | 2.77 (m, 1H). |
| 012 | 348.2 | L2 | 2.49 | (DMSO-d₆) δ ppm: 8.37-8.38 (d, 1H), 8.24-8.25 (d, 1H), 7.44 |
| | | | | (s, 1H), 7.27-7.29 (m, 3H), 7.17-7.19 (t, 1H), 7.07-7.11 (t, 1H), |
| | | | | 5.97-6.08 (m, 2H), 5.49-5.52 (d, 1H), 4.15-4.19 (s, 1H), 3.86- |
| | | | | 3.90 (m, 1H), 3.63-3.68 (m, 1H), 3.07-3.13 (m, 1H), 2.67-2.78 |
| | | | | (m, 1H). |
| 013 | 285.2 | L2 | 3.00 | (DMSO-d₆) δ ppm: 8.03-8.04 (d, 1H), 6.97-7.01 (t, 1H), 6.90- |
| | | | | 6.92 (d, 1H), 6.68-6.70 (d, 1H), 6.48-6.52 (t, 1H), 6.23-6.31 (m, |
| | | | | 1H), 6.07-6.12 (dd, 1H), 5.56-5.59 (dd, 1H), 4.02-4.06 (m, 1H), |
| | | | | 3.57 (s, 1H), 3.29-3.31 (d, 1H), 2.88-2.93 (m, 2H), 2.60-2.67 |
| | | | | (m, 1H), 1.60-1.79 (m, 5H), 1.34-1.45 (m, 4H), 1.21 (s, mH). |
| 014 | 336.2 | L2 | 2.26 | (DMSO-d₆) δ ppm: 8.27-8.28 (d, 1H), 8.20-8.22 (d, 1H), 7.76- |
| | | | | 7.79 (d, 2H), 7.22-7.24 (d, 2H), 7.12-7.14 (d, 1H), 6.98-7.03 (m, |
| | | | | 1H), 6.88-6. 90 (d, 1H), 6.79-6.83 (t, 1H), 6.18-6.25 (m, 1H), |
| | | | | 6.02-6.07 (dd, 1H), 5.54-5.57 (dd, 1H), 4.17-4.21 (m, 1H), 3.79- |
| | | | | 3.83 (dd, 1H), 3.39-3.44 (m, 1H), 3.04-3.10 (dd, 1H), 2.74-2. |
| | | | | 80 (m, 4H). |
| 015 | 336.3 | L2 | 2.31 | (DMSO-d₆) δ ppm: 9.91 (s, 1H), 8.19-8.21 (d, 1H), 7.503-7.508 |
| | | | | (d, 1H), 7.23-7.29 (m, 2H), 7.06-7.08 (d, 1H), 6.87-6.96 (m, |
| | | | | 2H), 6.72-6.68 (m, 2H), 6.29-6.22 (m, 1H), 6.05-6.09 (m, 1H), |
| | | | | 5.595 (dd, 1H), 4.18-4.22 (m, 1H), 3.68-3.72 (m, 1H), 3.34-3.36 |
| | | | | (m, 1H), 3.03-3.08 (m, 1H), 2.75-2.81 (m, 1H), 2.01 (s, 3H). |
| 016 | 318.2 | L2 | 2.64 | (DMSO-d₆) δ ppm: 11.12 (s, 1H), 8.18-8.20 (d, 1H), 7.35-7.36 |
| | | | | (t, 1H), 7.399-7.431 (m, 2H), 6.96-7.00 (m, 2H), 6.79-6.83 (m, |
| | | | | 1H), 6.54-6.58 (m, 1H), 6.39-6.40 (t, 1H), 6.28-6.33 (m, 2H), |
| | | | | 6.06-6.10 (dd, 1H), 5.56-5.59 (dd, 1H), 4.26-4.34 (m, 1H), 3.65- |
| | | | | 3.69 (dd, 1H), 3.39-3.44 (m, 1H), 3.05-3.10 (dd, 1H), 2.78-2.84 |
| | | | | (m, 1H). |
| 017 | 318.2 | L2 | 2.69 | (DMSO-d₆) δ ppm: 11.04 (s, 1H), 8.19-8.21 (d, 1H), 7.52-7.54 |
| | | | | (d, 1H), 7.32-7.33 (m, 1H), 7.234-7.236 (d, 1H), 7.01-7.03 (d, |
| | | | | 1H), 6.82-6.90 (m, 2H), 6.58-6.62 (m, 1H), 6.40-6.42 (m, 2H), |
| | | | | 6.28-6.35 (m, 2H), 6.05-6.10 (dd, 1H), 5.56-5.59 (dd, 1H), 4.25- |
| | | | | 4.33 (m, 1H), 3.69-3.72 (m, 1H), 3.40-3.45 (m, 1H), 3.06-3.11 |
| | | | | (m, 1H), 2.78-2.84 (m, 1H). |
| 018 | 315.3 | L1 | 2.40 | (CDCl₃) δ ppm: 7.11-7.18 (m, 1H), 6.99-7.08 (m, 3H), 6.81-6.95 |
| | | | | (m, 1H), 6.77-6.79 (m, 1H), 6.65-6.67 (d, 1H), 6.24-6.29 (dd, |
| | | | | 1H), 5.99-6.06 (m, 1H), 5.76-5.82 (br d, 1H), 5.62-5.65 (dd, |
| | | | | 1H), 4.61-4.66 (m, 1H), 3.72-3.76 (m, 1H), 3.54-3.59 (m, 1H), |
| | | | | 3.20-3.25 (m, 1H), 2.83-2.87 (m, 1H). |
| 019 | 361.3 | L2 | 3.52 | (CDCl₃) δ ppm: 7.20-7.22 (d, 2H), 7.11-7.13 (d, 2H), 7.03-7.05 |
| | | | | (d, 1H), 6.94-6.98 (t, 1H), 6.70-6.74 (t, 1H), 6.63-6.66 (d, 1H), |
| | | | | 6.23-6.27 (dd, 1H), 5.96-6.06 (m, 2H), 5.59-5.62 (dd, 1H), 4.65- |
| | | | | 4.66 (m, 1H), 3.76-3.79 (dd, 1H), 3.53-3.57 (m, 1H), 3.20-3.26 |
| | | | | (dd, 1H), 2.85-2.89 (d, 1H), 2.49-2.50 (d, 1H), 1.84-1.90 (m, |
| | | | | 4H), 1.73-1.77 (d, 1H), 1.38-1.43 (m, 4H), 1.26-1.27 (m, 1H). |
| 020 | 329.2 | L2 | 2.75 | (DMSO-d₆) δ ppm: 7.46-7.48 (d, 2H), 7.26-7.29 (d, 2H), 7.09- |
| | | | | 7.11 (d, 1H), 7.00-7.04 (m, 1H), 6.82-6.88 (m, 2H), 6.48-6.81 |
| | | | | (m, 1H), 6.20-6.24 (dd, 1H), 6.02-5.95 (m, 1H), 5.74-5.76 (d, |
| | | | | 1H), 5.58-5.61 (dd, 1H), 4.63-4.67 (m, 1H), 3.79-3.83 (m, 1H), |
| | | | | 3.65-3.70 (m, 1H), 3.19-3.25 (m, 1H), 2.84-2.88 (m, 1H). |
| 021 | 363.2 | L2 | 3.06 | (DMSO-d₆) δ ppm: 7.21-7.26 (m, 4H), 7.07-7.09 (d, 1H), 6.98- |
| | | | | 7.02 (m, 1H), 6.77-6.81 (m, 1H), 6.69-6.72 (d, 1H), 6.22-6.27 |
| | | | | (dd, 1H), 5.98-6.05 (m, 1H), 5.80-5.81 (d, 1H), 5.60-5.63 (dd, |
| | | | | 1H), 4.62-4.65 (m, 1H), 3.76-3.79 (m, 1H), 3.61-3.62 (m, 1H), |
| | | | | 3.21-3.26 (m, 1H), 2.84-2.88 (m, 1H). |
| 022 | 345.2 | L2 | 2.85 | (CDCl₃) δ ppm: 7.19-7.22 (m, 2H), 7.12-7.15 (m, 2H), 7.06-7.08 |
| | | | | (d, 1H), 6.97-7.01 (m, 1H), 6.74-6.78 (m, 1H), 6.32-6.51 (m, |
| | | | | 2H), 6.24-6.28 (dd, 1H), 6.00-6.06 (m, 1H), 5.85-5.87 (br d, |
| | | | | 1H), 5.61-5.64 (dd, 1H), 4.64-4.68 (m, 1H), 3.75-3.79 (m, 1H), |
| | | | | 3.55-3.60 (m, 1H), 3.21-3.27 (m, 1H), 2.84-2.89 (m, 1H). |
| 023 | 405.1 | L2 | 3.02 | (CDCl₃) δ ppm: 7.64-7.68 (d, 2H), 7.20-7.26 (d, 2H), 7.06-7.14 |
| | | | | (d, 1H), 6.91-6.92 (m, 2H), 6.88-6.90 (m, 1H), 6.18-6.22 (dd, |
| | | | | 1H), 5.92-5.98 (m, 1H), 5.58-5.61 (m, 2H), 4.61-4.65 (m, 1H), |
| | | | | 3.80-3.84 (m, 1H), 3.71-3.74 (m, 1H), 3.17-3.23 (m, 1H), 2.81- |
| | | | | 2.86 (m, 1H). |
| 024 | 297.2 | L2 | 2.71 | (CDCl₃) δ ppm: 7.26-7.30 (m, 1H), 7.07-7.09 (d, 1H), 6.97-7.02 |
| | | | | (m, 2H), 6.91-6.94 (m, 1H), 6.79-6.85 (m, 3H), 6.21-6.25 (dd, |
| | | | | 1H), 5.96-6.03 (m, 1H), 5.75-5.83 (d, 1H), 5.59-5.62 (dd, 1H), |
| | | | | 4.63-4.67 (m, 1H), 3.76-3.80 (m, 1H), 3.61-3.65 (m, 1H), 3.19- |
| | | | | 3.24 (m, 1H), 2.83-2.84 (m, 1H). |
| 025 | 315.2 | L2 | 2.67 | (CDCl₃) δ ppm: 6.98-7.12 (m, 5H), 6.76-6.80 (m, 1H), 6.41-6.43 |
| | | | | (dd, 1H), 6.23-6.24 (dd, 1H), 6.00-6.07 (m, 2H), 5.60-5.63 (dd, |
| | | | | 1H), 4.64-4.69 (m, 1H), 3.80-3.83 (dd, 1H), 3.50-3.54 (m, 1H), |
| | | | | 3.24-3.29 (dd, 1H), 2.88-2.93 (dd, 1H). |
| 026 | 315.2 | L2 | 2.77 | (DMSO-d₆) δ ppm: 7.11-7.14 (m, 2H), 7-7.09 (m, 2H), 6.98- |
| | | | | 6.99 (m, 1H), 6.91-6.94 (m, 1H), 6.43-6.45 (d, 1H), 6.22-6.27 |
| | | | | (dd, 1H), 5.99-6.06 (m, 2H), 5.60-5.63 (dd, 1H), 4.66-4.67 (m, |
| | | | | 1H), 3.78-3.81 (m, 1H), 3.52-3.54 (m, 1H), 3.49-3.51 (m, 1H), |
| | | | | 3.23-3.28 (m, 1H), 2.87-2.91 (m, 1H). |
| 027 | 315.1 | L2 | 2.75 | (CDCl₃) δ ppm: 7.05-7.12 (m, 2H), 6.98-7.00 (dd, 1H), 6.86- |
| | | | | 6.90 (m, 1H), 6.70-6.72 (m, 2H), 6.47-6.51 (m, 1H), 6.20-6.24 |
| | | | | (dd, 1H), 5.94-6.01 (q, 1H), 5.60-5.62 (m, 2H), 4.61-4.65 (m, |
| | | | | 1H), 3.75-3.78 (dd, 1H), 3.62-3.67 (m, 1H), 3.16-3.22 (dd, 1H), |
| | | | | 2.80-2.85 (dd, 1H). |
| 028 | 365.1 | L2 | 2.98 | (CDCl₃) δ ppm: 7.45-7.49 (t 1H), 7.09-7.26 (m, 3H), 6.93-7.00 |
| | | | | (m, 3H), 6.18-6.22 (dd, 1H), 5.91-5.98 (m, 1H), 5.58-5.61 (dd, |
| | | | | 1H), 5.54-5.55 (d, 1H), 4.60-4.62 (m, 1H), 3.80-3.84 (m, 1H), |
| | | | | 3.71-3.75 (m, 1H), 3.16-3.21 (m, 1H), 2.80-2.85 (m, 1H). |
| 029 | 365.1 | L1 | 2.91 | (CDCl₃) δ ppm: 7.37-7.45 (m, 3H), 7.09-7.10 (d, 1H), 7.00-7.04 |
| | | | | (m, 1H), 6.80-6.84 (m, 1H), 6.46-6.48 (d, 1H), 6.22-6.26 (dd, |
| | | | | 1H), 5.98-6.05 (m, 1H), 5.93-5.95 (d, 1H), 5.60-5.63 (dd, 1H), |
| | | | | 4.64-4.68 (m, 1H), 3.81-3.84 (m, 1H), 3.55-3.60 (m, 1H), 3.24- |
| | | | | 3.30 (m, 1H), 2.87-2.92 (m, 1H). |
| 030 | 379.2 | L2 | 3.11 | (CDCl₃) δ ppm: 7.55-7.5 (d, 2H), 7.20-7.24 (d, 2H), 7.11-7.13 |
| | | | | (d, 1H), 7.04-7.08 (m, 1H), 6.98-7.00 (m, 1H), 6.85-6.89 (m, |
| | | | | 1H), 6.16-6.21 (dd, 1H), 5.92-5.98 (m, 1H), 5.64-5.66 (d, 1H), |
| | | | | 5.57-5.60 (dd, 1H), 4.62-4.66 (m, 1H), 3.79-3.82 (m, 1H), 3.69- |
| | | | | 3.74 (m, 1H), 3.18-3.23 (m, 1H), 2.82-2.87 (m, 1H). |
| 031 | 349.1 | L2 | 2.49 | (CDCl₃) δ ppm: 8.70 (s, 2H), 7.20-7.22 (m, 1H), 7.14-7.18 (m, |
| | | | | 1H), 7.10-7.11 (m, 1H), 7.02-7.05 (m, 1H), 6.20-6.24 (dd, 1H), |
| | | | | 5.92-5.99 (m, 1H), 5.61-5.64 (dd, 1H), 5.49-5.50 (m, 1H), 4.63- |
| | | | | 4.65 (m, 1H), 3.82-3.90 (m, 1H), 3.79-3.81 (m, 1H), 3.19-3.24 |
| | | | | (m, 1H), 2.82-2.87 (dd, 1H). |
| 142 | 304.3 | L2 | 2.61 | (DMSO-d₆) δ ppm: 8.22 (d, 1H), 7.66 (d, 2H), 7.28 (d, 2H), 7.20 |
| | | | | (d, 1H), 7.07-7.09 (m, 2H), 6.92-6.96 (m, 1H), 6.13-6.20 (m, |
| | | | | 1H), 6.03 (dd, 1H), 5.54 (dd, 1H), 4.14-4.18 (m, 1H), 3.84-3.88 |
| | | | | (m, 1H), 3.46-3.51 (m, 1H), 3.05-3.10 (m, 1H), 2.73-2.79 (m, |
| | | | | 1H). |
| 032 | 313.2 | L2 | 2.93 | (DMSO-d₆) δ ppm: 8.23 (d, 1H), 7.31-7.35 (t, 1H), 7.24-7.25 |
| | | | | (m, 1H), 7.17-7.19 (m, 1H), 7.11-7.16 (m, 2H), 7.00-7.09 (t, |
| | | | | 1H), 6.77-6.81 (t, 2H), 6.20-6.27 (m, 1H), 6.02-6.07 (dd, 1H), |
| | | | | 5.54-5.57 (dd, 1H), 4.17-4.22 (m, 1H), 3.73-3.77 (dd, 1H), 3.38- |
| | | | | 3.43 (t, 1H), 3.05-3.10 (dd, 1H), 2.74-2.80 (m, 1H). |
| 033 | 313.2 | L2 | 2.91 | (DMSO-d₆) δ ppm: 8.19-8.21 (s, 1H), 7.35-7.38 (m, 2H), 7.22- |
| | | | | 7.25 (m, 2H), 7.08-7.10 (d, 1H), 6.94-6.98 (t, 1H), 6.68-6.77 (m, |
| | | | | 2H), 6.21-6.28 (m, 1H), 6.03-6.08 (dd, 1H), 5.55-5.58 (dd, 1H), |
| | | | | 4.18-4.22 (s, 1H), 3.69-3.73 (dd, 1H), 3.36-3.41 (m, 1H), 3.04- |
| | | | | 3.10 (dd, 1H), 2.74-2.80 (m, 1H). |
| 034 | 307.3 | L2 | 3.03 | (DMSO-d₆) δ ppm: 8.15-8.25 (d, 1H), 7.00-7.13 (m, 4H), 6.81- |
| | | | | 6.85 (m, 1H), 6.54-6.57 (m, 1H), 6.24-6.34 (m, 1H), 6.04-6.10 |
| | | | | (dd, 1H), 5.87-5.89 (d, 1H), 5.56-5.59 (dd, 1H), 4.25-4.32 (m |
| | | | | 1H), 3.46-3.51 (m, 1H), 3.35-3.37 (m, 1H), 3.03-3.07 (m, 1H), |
| | | | | 2.74-2.80 (m, 1H), 2.28 (s, 3H), 2.07 (s, 3H). |
| 035 | 297.3 | L2 | 2.35 | (DMSO-d₆) δ ppm: 8.18-8.20 (d, 1H), 7.18-7.27 (m, 4H), 7.06- |
| | | | | 7.07 (d, 1H), 6.91-6.95 (t, 1H), 6.67-6.71 (t, 1H), 6.48-6.50 (d, |
| | | | | 1H), 6.23-6.30 (q, 1H), 6.06-6.11 (dd, 1H), 5.59-5.62 (dd, 1H), |
| | | | | 4.22-4.26 (m, 1H), 3.64-3.68 (m, 1H), 3.36-3.41 (m, 1H), 3.05- |
| | | | | 3.10 (dd, 1H), 2.77-2.83 (m, 1H). |
| 036 | 315.2 | L2 | 2.81 | (DMSO-d₆) δ ppm: 8.18-8.20 (d, 1H), 7.35-7.46 (m, 2H), 7.14- |
| | | | | 7.15 (m, 1H), 7.04-7.06 (d, 1H), 6.81-6.91 (m, 1H), 6.65-6.69 |
| | | | | (m, 1H), 6.25-6.31 (m, 1H), 6.17-6.19 (d, 1H), 6.05-6.10 (dd, |
| | | | | 1H), 5.56-5.59 (dd, 1H), 4.24-4.29 (m, 1H), 3.55-3.59 (m, 1H), |
| | | | | 3.37-3.42 (m, 1H), 3.06-3.11 (m, 1H), 2.78-2.84 (m, 1H). |
| 037 | 349.2 | L2 | 3.02 | (DMSO-d₆) δ ppm: 7.86-7.88 (m, 1H), 7.57-7.63 (m, 2H), 7.30- |
| | | | | 7.35 (m, 2H), 7.14-7.18 (m, 1H), 6.98-7.06 (m, 2H), 6.86-6.90 |
| | | | | (m, 1H), 4.02-4.11 (m, 1H), 3.73-3.79 (m, 1H), 3.45-3.53 (m, |
| | | | | 1H), 3.00-3.09 (m, 1H), 2.68-2.77 (m, 1H), 1.90-2.02 (m, 2H), |
| | | | | 0.80-0.87 (m, 3H). |
| 038 | 375.2 | L2 | 3.21 | (DMSO-d₆) δ ppm: 7.84 (d, 1H), 7.60 (d, 2H), 7.33 (d, 2H), 7.16 |
| | | | | (d, 1H), 6.97-7.06 (m, 2H), 6.85-6.89 (m, 1H), 5.61 (s, 1H), |
| | | | | 4.06-4.15 (m, 1H), 3.80-3.83 (dd, 1H), 3.38-3.43 (m, 1H), 3.0- |
| | | | | 3.05 (dd, 1H), 2.70-2.76 (m, 1H), 2.05 (s, 3H), 1.74 (s, 3H). |
| 039 | 365.2 | L2 | 3.19 | (CDCl₃) δ ppm: 7.56 (d, 2H), 7.27 (d, 2H), 7.12 (d, 1H), 7.03- |
| | | | | 7.05 (m, 1H), 6.94 (d, 1H), 6.86-6.90 (m, 1H), 6.41 (d, 1H), 5.63 |
| | | | | (dd, 1H), 5.07 (dd, 1H), 4.59-4.62 (m, 1H), 3.82-3.86 (m, 1H), |
| | | | | 3.68-3.72 (m, 1H), 3.21-3.26 (m, 1H), 2.86-2.91 (m, 1H). |
| 040 | 345.2 | L2 | 3.03 | (DMSO-d₆) δ ppm: 8.96-8.94 (d, 1H), 7.61-7.63 (d, 2H), 7.33- |
| | | | | 7.35 (d, 2H), 7.14-7.16 (m, 1H), 7.02-7.06 (m, 1H), 6.96-6.98 |
| | | | | (m, 1H), 6.86-6.90 (m, 1H), 4.09-4.16 (m, 1H), 3.81-3.85 (m, |
| | | | | 1H), 3.40-3.45 (m, 1H), 3.00-3.06 (m, 1H), 2.76-2.82 (m, 1H). |
| 041 | 404.3 | L2 | 2.36 | (DMSO-d₆) δ ppm: 8.10-8.12 (d, 1H), 7.58-7.61 (d, 2H), 7.33- |
| | | | | 7.35 (d, 2H), 7.15-7.17 (d, 1H), 7.02-7.06 (m, 1H), 6.92-6.97 |
| | | | | (m, 1H), 6.86-6.90 (m, 1H), 6.45-6.52 (m, 1H), 5.98-6.02 (m, |
| | | | | 1H), 4.13-4.18 (m, 1H), 3.80-3.84 (dd, 1H), 3.44-3.49 (m, 1H), |
| | | | | 3.04-3. 10 (dd, 1H), 2.91-2.93 (m, 2H), 2.72-2.79 (m, 1H), 2.09 |
| | | | | (s, 6H). |
| 042 | 372.2 | L3 | 5.25, | (DMSO-d₆) δ ppm: 7.59-7.66 (m, 2H), 7.32-7.38 (m, 2H), 7.16- |
| | | | 5.27 | 7.21 (m, 1H), 7.01-7.06 (m, 1H), 6.95 (d, 1H), 6.85-6.89 (m, |
| | | | | 1H), 4.37-4.41 (m, 1H), 4.10-4.13 (m, 1H), 3.78-3.86 (m, 2H), |
| | | | | 3.43-3.48 (m, 2H), 3.05-3.11 (m, 2H). (mixture of |
| | | | | diastereoisomers) |
| 043 | 337.2 | L2 | 2.26 | (DMSO-d₆) δ ppm: 7.58-7.64 (m, 2H), 7.33-7.37 (m, 2H), 7.11- |
| | | | | 7.16 (m, 1H), 6.96-7.03 (m, 2H), 6.82-6.86 (m, 1H), 4.42-4.47 |
| | | | | (m, 1H), 3.82-3.87 (m, 1H), 3.37-3.43 (m, 2H), 3.25-3.31 (m, |
| | | | | 1H), 2.98-3.05 (m, 2H), 2.54-2.66 (m, 3H), 1.73-1.81 (m, 1H). |
| 043-En1 | 337.2 | S2 | 4.22 | (DMSO-d₆) δ ppm: 7.59-7.64 (m, 2H), 7.32-7.36 (m, 2H), 7.12- |
| | | | | 7.16 (m, 1H), 6.95-7.02 (m, 2H), 6.81-6.86 (m, 1H), 4.42-4.46 |
| | | | | (m, 1H), 3.82-3.88 (m, 1H), 3.36-3.43 (m, 2H), 3.24-3.28 (m, |
| | | | | 1H), 2.98-3.05 (m, 2H), 2.55-2.69 (m, 3H), 1.70-1.82 (m, 1H). |
| 043-En2 | 337.2 | S2 | 4.21 | (DMSO-d₆) δ ppm: 7.59-7.63 (m, 2H), 7.32-7.37 (m, 2H), 7.12- |
| | | | | 7.16 (m, 1H), 6.97-7.03 (m, 2H), 6.82-6.86 (m, 1H), 4.43-4.49 |
| | | | | (m, 1H), 3.82-3.87 (m, 1H), 3.36-3.42 (m, 2H), 3.23-3.28 (m, |
| | | | | 1H), 2.97-3.04 (m, 2H), 2.54-2.65 (m, 3H), 1.71-1.99 (m, 1H). |
| 044 | 351.2 | L2 | 2.39 | (DMSO-d₆) δ ppm: 7.59-7.67 (m, 2H), 7.32-7.38 (m, 2H), 7.13- |
| | | | | 7.18 (m, 1H), 6.99-7.04 (m, 1H), 6.92-6.97 (m, 1H), 6.83-6.89 |
| | | | | (m, 1H), 3.90-3.97 (m, 1H), 3.36-3.42 (m, 1H), 3.24-3.28 (m, |
| | | | | 3H), 3.04-3.11 (m, 1H), 2.67-2.74 (m, 1H). |
| 045 | 351.2 | L2 | 2.24 | (DMSO-d₆) δ ppm: 7.59-7.63 (m, 2H), 7.32-7.36 (m, 2H), 7.12- |
| | | | | 7.16 (m, 1H), 6.96-7.03 (m, 2H), 6.83-6.88 (m, 1H), 4.31-4.45 |
| | | | | (m, 1H), 3.81-3.88 (m, 1H), 3.38-3.43 (m, 2H), 3.23-3.28 (m, |
| | | | | 1H), 2.93-3.02 (m, 2H), 2.55-2.69 (m, 3H), 1.66-1.88 (m, 1H), |
| | | | | 1.47-1.55 (m, 2H). |
| 045-En1 | 351.2 | S2 | 2.20 | (DMSO-d₆) δ ppm: 7.58-7.63 (m, 2H), 7.31-7.37 (m, 2H), 7.11- |
| | | | | 7.16 (m, 1H), 6.97-7.04 (m, 2H), 6.82-6.88 (m, 1H), 4.33-4.47 |
| | | | | (m, 1H), 3.81-3.87 (m, 1H), 3.39-3.44 (m, 2H), 3.22-3.27 (m, |
| | | | | 1H), 2.94-3.03 (m, 2H), 2.54-2.68 (m, 3H), 1.65-1.89 (m, 1H), |
| | | | | 1.47-1.54 (m, 2H). |
| 045-En2 | 351.2 | S2 | 2.22 | (DMSO-d₆) δ ppm: 7.59-7.63 (m, 2H), 7.32-7.36 (m, 2H), 7.11- |
| | | | | 7.16 (m, 1H), 6.97-7.03 (m, 2H), 6.82-6.87 (m, 1H), 4.36-4.41 |
| | | | | (m, 1H), 3.82-3.87 (m, 1H), 3.36-3.45 (m, 2H), 3.22-3.27 (m, |
| | | | | 1H), 2.92-3.03 (m, 2H), 2.52-2.64 (m, 3H), 1.70-1.78 (m, 1H), |
| | | | | 1.46-1.55 (m, 2H). |
| 046 | 365.2 | L2 | 2.32 | (DMSO-d₆) δ ppm: 7.59-7.65 (m, 2H), 7.33-7.38 (m, 2H), 7.12- |
| | | | | 7.18 (m, 1H), 6.94-7.04 (m, 2H), 6.81-6.87 (m, 1H), 3.83-3.91 |
| | | | | (m, 1H), 3.28-3.33 (m, 1H), 3.01-3.10 (m, 2H), 2.74-2.85 (m, |
| | | | | 2H), 2.54-2.63 (m, 1H), 2.24-2.30 (m, 2H). |
| 047 | 371.2 | L2 | 2.98 | (DMSO-d₆) δ ppm: 7.64 (d, 2H), 7.36 (d, 3H), 7.15 (d, 1H), |
| | | | | 6.95-7.05 (m, 2H), 6.84-6.88 (m, 1H), 3.90-3.94 (dd, 1H), 3.72- |
| | | | | 3.77 (m, 1H), 3.36-3.41 (m, 1H), 3.09-3.14 (dd, 1H), 2.96 (s, |
| | | | | 3H), 2.67-2.79 (m, 1H). |
| 048 | 383.2 | L2 | 3.11 | (DMSO-d₆) δ ppm: 7.62-7.65 (d, 3H), 7.33-7.36 (d, 2H), 7.12- |
| | | | | 7.14 (d, 1H), 7.01-7.05 (m, 1H), 6.93-6.95 (m, 1H), 6.76-6.87 |
| | | | | (m, 2H), 6.00 (d, 1H), 5.91-5.94 (d, 1H), 3.86-3.90 (dd, 1H), |
| | | | | 3.55-3.58 (m, 1H), 3.37-3.42 (m, 1H), 3.04-3.09 (dd, 1H), 2.74- |
| | | | | 2.80 (m, 1H). |
| 049 | 399.2 | L2 | 2.34 | (DMSO) δ ppm: 7.60-7.62 (d, 2H), 7.34-7.36 (d, 2H), 7.13-7.14 |
| | | | | (d, 1H), 6.95-7.03 (m, 2H), 6.82-6.86 (m, 1H), 3.81-3.84 (m, |
| | | | | 1H), 3.18-3.29 (m, 1H), 3.15-3.16 (m, 2H), 2.93-3.00 (m, 7H), |
| | | | | 2.49-2.50 (m, 1H). |
| 050 | 383.2 | L2 | 3.16 | (DMSO-d₆) δ ppm: 7.65-7.67 (d, 2H), 7.36-7.38 (d, 2H), 7.14- |
| | | | | 7.15 (m, 1H), 7.00-7.04 (m, 1H), 6.89-6.91 (m, 1H), 6.82-6.86 |
| | | | | (m, 1H), 4.12-4.15 (t, 2H), 3.82-3.85 (m, 1H), 3.70-3.75 (m, |
| | | | | 1H), 3.62-3.66 (m, 1H), 3.10-3.16 (m, 2H), 3.05-3.06 (m, 1H), |
| | | | | 2.84-2.90 (m, 1H). |
| 051 | 414.3 | L3 | 3.73 | (DMSO-d₆) δ ppm: 7.60-7.62 (d, 2H), 7.34-7.36 (d, 2H), 7.13- |
| | | | | 7.14 (d, 1H), 6.95-7.02 (m, 2H), 6.76-6.86 (m, 2H), 3.79-3.83 |
| | | | | (m, 1H), 3.09 (m, 1H), 2.99-3.09 (m, 4H), 2.86-2.93 (m, 2H), |
| | | | | 2.50-2.56 (m, 4H). |
| 052 | 360.2 | L2 | 2.96 | (CDCl3) δ ppm: 7.58-7.60 (d, 2H), 7.29-7.31 (d, 2H), 7.11-7.13 |
| | | | | (d, 1H), 7.03-7.08 (t, 1H), 6.87-6.93 (m, 2H), 6.28-6.29 (d, 1H), |
| | | | | 4.53-4.56 (m, 1H), 3.70-3.82 (m, 2H), 3.18-3.31 (m, 3H), 2.86- |
| | | | | 2.91 (m, 1H). |
| 053 | 346.2 | L2 | 2.31 | (CDCl₃) δ ppm: 7.53-7.56 (d, 2H), 7.27-7.29 (d, 2H), 7.10-7.12 |
| | | | | (m, 1H), 6.99-7.04 (m, 1H), 6.95-6.97 (m, 1H), 6.83-6.87 (m, |
| | | | | 1H), 3.77-3.81 (m, 1H), 3.43-3.47 (m, 1H), 3.22-3.24 (m, 1H), |
| | | | | 3.07-3.17 (m, 1H), 2.88-3.01 (m, 2H), 2.68-2.78 (m, 1H), 2.44- |
| | | | | 2.49 (t, 2H), 1.2-1.4 (br s, 1H). |
| 054 | 332.2 | L2 | 3.09 | (CDCl₃) δ ppm: 7.54-7.56 (d, 2H), 7.27-7.29 (d, 2H), 7.10-7.12 |
| | | | | (d, 1H), 7.00-7.04 (m, 1H), 6.94-6.96 (m, 1H), 6.83-6.87 (m, |
| | | | | 1H), 3.78-3.79 (m, 1H), 3.55-3.60 (m, 3H), 3.44-3.54 (m, 1H), |
| | | | | 3.11-3.16 (m, 1H), 2.73-2.78 (m, 1H), 1.38-1.49 (m, 1H). |
| 055 | 471.2 | L3 | 6.69 | (DMSO-d₆) δ ppm: 7.63 (d, 2H), 7.36 (d, 2H), 7.15-7.17 (dd, |
| | | | | 1H), 7.05 (d, 1H), 6.91-6.98 (m, 2H), 3.69-3.82 (m, 2H), 3.49- |
| | | | | 3.54 (m, 1H), 2.96-3.02 (m, 1H), 2.64-2.70 (m, 1H), 1.28 (s, |
| | | | | 9H). |
| 056 | 411.2 | L6 | 2.55 | / |
| 057 | 471.2 | L4 | 3.09 | / |
| 058 | 425.2 | L1 | 2.72 | (DMSO-d₆) δ ppm: 8.26 (d, 1H), 7.64 (d, 2H), 7.37 (d, 2H), 7.18 |
| | | | | (d, 1H), 6.92-7.00 (m, 2H), 6.14-6.20 (m, 1H), 5.98-6.03 (dd, |
| | | | | 1H), 5.51-5.54 (dd, 1H), 4.17-4.12 (m, 1H), 3.78-3.81 (dd, 1H), |
| | | | | 3.50-3.55 (m, 1H), 3.06-3.11 (dd, 1H), 2.69-2.75 (dd, 1H). |
| 059 | 438.2 | L2 | 2.41 | (DMSO-d6) δ ppm: 8.46-8.47 (d, 1H), 8.42-8.44 (m, 1H), 8.23- |
| | | | | 8.24 (d, 1H), 7.55-7.61 (m, 3H), 7.32-7.35 (m, 3H), 7.01-7.03 |
| | | | | (m, 1H), 6.89-6.91 (d, 1H), 6.73-6.75 (d, 1H), 6.15-6.21 (m, |
| | | | | 1H), 6.00-6.04 (dd, 1H), 5.52-5.56 (dd, 1H), 4.11-4.20 (m, 1H), |
| | | | | 3.98-4.02 (s, 2H), 3.80-3.84 (m, 1H), 3.38-3.43 (m, 1H), 3.00- |
| | | | | 3.06 (m, 1H), 2.60-2.66 (m, 1H). |
| 060 | 365.2 | L2 | 3.08 | (CDCl₃) δ ppm: 7.58 (d, 2H), 7.29 (d, 2H), 6.95-7.00 (m, 1H), |
| | | | | 6.65 (d, 1H), 6.58 (td, 1H), 6.22 (dd, 1H), 5.96-6.03 (m, 1H), |
| | | | | 5.66 (d, 1H), 5.61 (dd, 1H), 4.61-4.65 (m, 1H), 3.79-3.83 (m, |
| | | | | 1H), 3.67-3.71 (m, 1H), 3.10-3.15 (m, 1H), 2.86-2.91 (m, 1H). |
| 061 | 440.3 | L2 | 2.36 | (DMSO-d6) δ ppm: 8.43-8.46 (m, 2H), 7.84-7.86 (d, 1H), 7.55- |
| | | | | 7.60 (m, 3H), 7.30-7.35 (m, 3H), 7.00-7.04 (m, 1H), 6.90-6.91 |
| | | | | (d, 1H), 6.73-6.74 (d, 1H), 4.05-4.06 (m, 1H), 3.97 (s, 2H), 3.70- |
| | | | | 3.73 (m, 1H), 3.41-3.46 (m, 1H), 2.94-3.00 (m, 1H), 2.56-2.62 |
| | | | | (m, 1H), 1.90-2.02 (m, 2H), 0.82-0.85 (t, 3H). |
| 062 | 462.2 | L2 | 3.14 | (CDCl₃) δ ppm: 7.57-7.59 (d, 2H), 7.49-7.52 (m, 1H), 7.37-7.41 |
| | | | | (m, 3H), 7.28-7.30 (d, 2H), 7.02-7.28 (t, 1H), 6.88-6.90 (d, 1H), |
| | | | | 6.69-6.71 (d, 1H), 6.16-6.20 (dd, 1H), 5.90-5.97 (m, 1H), 5.61- |
| | | | | 5.63 (d, 1H), 5.59-5.61 (dd, 1H), 4.58-4.60 (m, 1H), 3.97 (s, |
| | | | | 2H), 3.76-3.80 (m, 1H), 3.60-3.65 (m, 1H), 2.86-2.92 (m, 1H), |
| | | | | 2.68-2.74 (m, 1H). |
| 063 | 455.3 | L2 | 3.37 | (CDCl₃) δ ppm: 7.51-7.54 (d, 2H), 7.22-7.28 (m, 3H), 6.99-7.05 |
| | | | | (m, 1H), 6.87-6.95 (m, 3H), 6.79-6.82 (d, 1H), 6.74-7.5 (d, 1H), |
| | | | | 6.14-6.19 (dd, 1H), 5.86-5.93 (m, 1H), 5.55-5.59 (m, 2H), 4.56- |
| | | | | 4.59 (m, 1H), 3.95 (s, 2H), 3.75-3.79 (m, 1H), 3.61-3.66 (m, |
| | | | | 1H), 2.89-2.94 (m, 1H), 2.71-2.76 (m, 1H). |
| 064 | 361.2 | L2 | 3.13 | (DMSO-d₆) δ ppm: 8.22 (d, 1H), 7.58 (d, 2H), 7.30 (d, 2H), |
| | | | | 6.94-6.98 (t, 1H), 6.79-6.86 (m, 2H), 6.14-6.19 (m, 1H), 5.99- |
| | | | | 6.04 (m, 1H), 5.51-5.55 (dd, 1H), 4.12-4.18 (m, 1H), 3.82-3.86 |
| | | | | (dd, 1H), 3.41-3.46 (m, 1H), 2.97-3.01 (dd, 1H), 2.56-2.62 (dd, |
| | | | | 1H), 2.21 (s, 3H). |
| 065 | 361.2 | L2 | 3.09 | (DMSO-d₆) δ ppm: 7.51-7.54 (d, 2H), 7.23-7.26 (d, 2H), 6.86- |
| | | | | 6.94 (m, 3H), 6.16-6.21 (dd, 1H), 5.90-5.97 (m, 1H), 5.64-5.66 |
| | | | | (d, 1H), 5.56-5.59 (dd, 1H), 4.60-4.64 (m, 1H), 3.77-3.81 (m, |
| | | | | 1H), 3.68-3.73 (m, 1H), 3.14-3.19 (m, 1H), 2.78-2.83 (m, |
| | | | | 1H).2.27 (s, 3H). |
| 066 | 437.2 | L2 | 3.38 | (DMSO-d₆) δ ppm: 7.60 (d, 2H), 7.29-7.34 (m, 4H), 7.18-7.23 |
| | | | | (m, 3H), 7.00-7.04 (t, 1H), 6.88 (d, 1H), 6.74 (d, 1H), 6.14-6.21 |
| | | | | (m, 1H), 6.00-6.05 (m, 1H), 5.54-5.57 (dd, 1H), 4.13-4.15 (m, |
| | | | | 1H), 3.95 (s, 2H), 3.78-3.82 (dd, 1H), 3.38-3.43 (m, 1H), 2.97- |
| | | | | 3.03 (dd, 1H), 2.61-2.67 (m, 1H). |
| 067 | 439.2 | L2 | 3.36 | (DMSO-d₆) δ ppm; 7.83-7.84 (d, 1H), 7.58-7.59 (d, 2H), 7.29- |
| | | | | 7.33 (m, 4H), 7.17-7.22 (m, 3H), 6.99-7.02 (t, 1H), 6.88-6.90 |
| | | | | (d, 1H), 6.71-6.73 (d, 1H), 4.03-4.04 (m, 1H), 3.94 (s, 2H), 3.69- |
| | | | | 3.73 (m, 1H), 3.40-3.45 (m, 1H), 2.91-2.97 (m, 1H), 2.56-2.63 |
| | | | | (m, 1H), 1.89-1.99 (m, 2H), 0.81-0.85 (t, 3H). |
| 068 | 446.3 | L2 | 2.25 | (CDCl₃) δ ppm: 7.51-7.55 (d, 2H), 7.25-7.27 (2H), 6.97-7.01 (m |
| | | | | 1H), 6.91-6.94 (m, 1H), 6.82-6.84 (m, 1H), 6.19-6.23 (dd, 1H), |
| | | | | 5.93-6.00 (m, 1H), 5.75-5.77 (d, 1H), 5.58-5.61 (dd, 1H), 4.65- |
| | | | | 4.69 (m, 1H), 3.81-3.85 (m, 1H), 3.66-3.70 (m, 5H), 3.36-3.48 |
| | | | | (m, 2H), 3.15-3.21 (m, 1H), 2.98-3.04 (m, 1H), 3.45-3.46 (m, |
| | | | | 4H). |
| 069 | 438.2 | L2 | 3.25 | (DMSO-d6) δ ppm: 8.24-8.26 (d, 1H), 7.59-7.61 (d, 2H), 7.34- |
| | | | | 7.37 (m, 3H), 7.20-7.22 (m, 2H), 6.94-6.98 (m, 3H), 6.77-6.80 |
| | | | | (m, 2H), 6.61-6.63 (d, 1H), 6.16-6.22 (m, 1H), 5.99-6.04 (dd, |
| | | | | 1H), 5.52-5.54 (dd, 1H), 4.17-4.21 (m, 1H), 3.81-3.85 (m, 1H), |
| | | | | 3.43-3.48 (m, 1H), 3.02-3.08 (m, 1H), 2.55-2.61 (m, 1H). |
| 070 | 439.2 | L2 | 2.33 | (DMSO-d₆) δ ppm: 8.24-8.28 (m, 2H), 7.98-8.00 (dd, 1H), 7.59- |
| | | | | 7.63 (m, 3H), 7.36-7.38 (d, 2H), 7.25-7.26 (m, 1H), 7.19-7.21 |
| | | | | (m, 1H), 6.97-7.01 (m, 1H), 6.76-6.78 (d, 1H), 6.66-6.68 (d, |
| | | | | 1H), 6.15-6.22 (m, 1H), 5.99-6.04 (dd, 1H), 5.52-5.55 (dd, 1H), |
| | | | | 4.18-4.22 (m, 1H), 3.81-3.85 m, 1H), 3.45-3.50 (m, 1H), 3.02- |
| | | | | 3.07 (m, 1H), 2.56-2.62 (m, 1H). |
| 071 | 440.3 | L2 | 3.26 | (DMSO-d₆) δ ppm: 7.55-7.57 (d, 2H), 7.27-7.31 (m, 4H), 6.92- |
| | | | | 7.01 (m, 4H), 6.81-6.83 (d, 1H), 6.61-6.63 (d, 1H), 5.54-5.60 |
| | | | | (d, 1H), 5.32 (s, 1H), 4.55-4.59 (m, 1H), 3.72-3.77 (m, 2H), |
| | | | | 2.96-3.01 (m, 1H), 2.66-2.71 (m, 1H), 2.00-2.12 (m, 2H), 0.99- |
| | | | | 1.03 (t, 3H). |
| 072 | 441.2 | L2 | 2.31 | (DMSO-d₆) δ ppm: 8.28 (d, 1H), 7.98-8.00 (dd, 1H), 7.89 (d, |
| | | | | 1H), 7.57-7.62 (t, 3H), 7.36 (d, 2H), 7.26-7.29 (m, 1H), 7.18- |
| | | | | 7.21 (m, 1H), 6.97-7.01 (t, 1H), 6.78 (d, 1H), 6.69 (d, 1H), 4.08- |
| | | | | 4.11 (m, 1H), 3.71-3.74 (m, 1H), 3.48-3.53 (m, 1H), 2.97-3.03 |
| | | | | (m, 1H), 2.51-2.58 (m, 1H), 1.90-2.02 (m, 2H), 0.81-0.85 (t, |
| | | | | 3H). |
| 073 | 377.1 | L2 | 2.98 | (DMSO-d₆) δ ppm: 8.16-8.18 (d, 1H), 7.58-7.60 (d, 2H), 7.32- |
| | | | | 7.34 (d, 2H), 7.00-7.04 (t, 1H), 6.55-6.59 (m, 2H), 6.13-6.20 (m, |
| | | | | 1H), 5.99-6.04 (dd, 1H), 5.50-5.53 (dd, 1H), 4.09-4.13 (m, 1H), |
| | | | | 3.79-3.82 (m, 4H), 3.42-3.47 (m, 1H), 2.93-2.99 (m, 1H), 2.54- |
| | | | | 2.60 (m, 1H). |
| 074 | 462.2 | L2 | 3.11 | (DMSO-d₆) δ ppm: 8.51 (s, 1H), 8.23-8.25 (d, 1H), 7.59-7.61 |
| | | | | (d, 2H), 7.32-7.34 (d, 2H), 6.94-7.02 (m, 2H), 6.76-6.79 (d, 1H), |
| | | | | 6.16-6.23 (m, 1H), 6.00-6.05 (dd, 1H), 5.53-5.56 (dd, 1H), 4.11- |
| | | | | 4.15 (m, 1H), 3.79-3.83 (m, 1H), 3.38-3.43 (m, 1H), 3.02-3.06 |
| | | | | (m, 1H), 2.55-2.61 (m, 1H), 1.46 (s, 9H). |
| 075 | 362.2 | L2 | 2.62 | (DMSO-d₆) δ ppm: 8.21-8.23 (d, 1H), 7.53-7.55 (d, 2H), 7.28- |
| | | | | 7.30 (d, 2H), 6.73-6.77 (m, 1H), 6.15-6.29 (m, 3H), 5.98-6.03 |
| | | | | (dd, 1H), 5.51-5.53 (dd, 1H), 4.93 (s, 2H), 4.14-4.18 (m, 1H), |
| | | | | 3.74-3.77 (m, 1H), 3.40-3.45 (m, 1H), 2.78-2.83 (m, 1H), 2.33- |
| | | | | 2.49 (m, 1H). |
| 075-En1 | 362.2 | S5 | 1.91 | (DMSO-d₆) δ ppm: 8.20-8.22 (d, 1H), 7.53-7.55 (d, 2H), 7.28- |
| | | | | 7.30 (d, 2H), 6.73-6.77 (t, 1H), 6.27-6.29 (dd, 1H), 6.21-6.23 |
| | | | | (dd, 1H), 6.14-6.19 (dd, 1H), 5.98-6.03 (dd, 1H), 5.50-5.53 (dd, |
| | | | | 1H), 4.93 (s, 2H), 4.14-4.18 (m, 1H), 3.74-3.77 (dd, 1H), 3.40- |
| | | | | 3.45 (dd, 1H), 2.78-2.83 (dd, 1H), 2.33-2.39 (dd, 1H). |
| 075-En1 | 362.2 | S5 | 2.83 | (DMSO-d₆) δ ppm: 8.20-8.22 (d, 1H), 7.53-7.55 (d, 2H), 7.28- |
| | | | | 7.30 (d, 2H), 6.73-6.77 (t, 1H), 6.27-6.29 (dd, 1H), 6.21-6.23 |
| | | | | (dd, 1H), 6.14-6.19 (dd, 1H), 5.98-6.03 (dd, 1H), 5.50-5.53 (dd, |
| | | | | 1H), 4.93 (s, 2H), 4.14-4.18 (m, 1H), 3.74-3.77 (dd, 1H), 3.40- |
| | | | | 3.45 (dd, 1H), 2.78-2.83 (dd, 1H), 2.33-2.39 (dd, 1H). |
| 076 | 404.2 | L2 | 2.58 | (DMSO-d₆) δ ppm: 9.30 (s, 1H), 8.25-8.27 (d, 1H), 7.60-7.62 |
| | | | | (d, 2H), 7.34-7.36 (d, 2H), 6.98-7.04 (m, 2H), 6.80-6.82 (dd, |
| | | | | 1H), 6.16-6.23 (m, 1H), 6.01-6.06 (m, 1H), 5.55-5.57 (dd, 1H), |
| | | | | 4.14-4.18 (m, 1H), 3.80-3.83 (m, 1H), 3.40-3.45 (m, 1H), 2.98- |
| | | | | 3.04 (m, 1H), 2.57-2.63 (m, 1H), 2.07 (s, 3H). |
| 077 | 420.3 | L2 | 2.90 | (DMSO-d₆) δ ppm: 8.21-8.23 (d, 1H), 7.54-7.56 (d, 2H), 7.23- |
| | | | | 7.29 (d, 2H), 6.87-6.91 (t, 1H), 6.29-6.30 (d, 1H), 6.24-6.26 (d, |
| | | | | 1H), 6.14-6.20 (q, 1H), 5.98-6.03 (dd, 1H), 5.50-5.54 (dd, 1H), |
| | | | | 4.77-4.80 (t, 1H), 4.14-4.19 (m, 1H), 3.76-3.79 (m, 1H), 3.53- |
| | | | | 3.55 (t, 2H), 3.41-3.47 (m, 1H), 3.26-3.32 (m, 5H), 2.77-2.83 |
| | | | | (m, 2H), 2.32-2.38 (m, 1H). |
| 078 | 372.2 | L2 | 2.94 | (CDCl₃) δ ppm: 7.61 (d, 2H), 7.27 (d, 2 H), 7.15-7.18 (m, 1H), |
| | | | | 7.04-7.11 (m, 2H), 6.26 (dd, 1H), 5.99-6.06 (m, 1H), 5.66 (dd, |
| | | | | 1H), 5.63 (d, 1H), 4.64-4.68 (m, 1H), 3.86-3.90 (m, 1H), 3.66- |
| | | | | 3.71 (m, 1H), 3.36-3.41 (m, 1H), 3.04-3.10 (m, 1H). |
| 079 | 391.3 | L4 | 1.95 | (DMSO-d₆) δ ppm: 12.95 (s, 1H), 8.23 (d, 1H), 7.62 (d, 2H), |
| | | | | 7.37-7.40 (m, 1H), 7.33 (d, 2H), 7.11-7.14 (m, 2H), 6.14-6.21 |
| | | | | (m, 1H), 6.01 (dd, 1H), 5.53 (dd, 1H), 4.11-4.21 (m, 1H), 3.82- |
| | | | | 3.86 (m, 1H), 3.36-3.50 (m, 2H), 2.99-3.05 (m, 1H). |
| 136 | 390.2 | L2 | 2.53 | (DMSO-d₆) δ ppm: 8.26 (d, 1H), 7.74 (s, 1H), 7.62 (d, 2H), 7.41 |
| | | | | (s, 1H), 7.33 (d, 2H), 7.08 t, 1H), 7.01 (d, 1H), 6.96 (d, 1H), |
| | | | | 6.16-6.23 (m, 1H), 6.04 (dd, 2H), 5.55 (dd, 1H), 4.10-4.15 (m, |
| | | | | 1H), 3.86-3.90 m, 1H), 3.36-3.41 (m, 1H), 3.13-3.19 (m, 1H), |
| | | | | 2.85-2.91 (m, 1H). |
| 137 | 415.3 | L2 | 2.65 | (DMSO-d₆) δ ppm: 8.25 (s, 1H), 7.65 (d, 2H), 7.14-7.40 (m, |
| | | | | 5H), 6.14-6.21 (m, 1H), 6.00 (dd, 1H), 5.52 (d, 1H), 4.16 (m, |
| | | | | 1H), 3.88 (d, 1H), 3.49 (t, 1H), 3.31 (m, 1H), 2.98 (m, 1H). |
| 138 | 376.3 | L2 | 2.21 | (DMSO-d₆) δ ppm: 8.21 (d, 1H), 7.58 (d, 2H), 7.30 (d, 2H), |
| | | | | 7.01-7.06 (m, 2H), 6.88-6.91 (m, 1H), 6.14-6.21 (m, 1H), 6.02 |
| | | | | (dd, 1H), 5.54 (dd, 1H), 4.09-4.17 (m, 1H), 3.83-3.87 (m, 1H), |
| | | | | 3.64-3.73 (m, 2H), 3.39-3.45 (m, 1H), 3.06-3.11 (m, 1H), 2.65- |
| | | | | 2.71 (m, 1H), 1.88-2.07 (m, 2H). |
| 139 | 377.3 | L2 | 2.68 | (DMSO-d₆) δ ppm: 8.22 (d, 1H), 7.58 (d, 2H), 7.30 (d, 2H), |
| | | | | 7.01-7.07 (m, 2H), 6.91 (dd, 1H), 6.15-6.21 (m, 1H), 6.02 (dd, |
| | | | | 1H), 5.53 (dd, 1H), 5.12 (s, 1H), 4.43-4.52 (m, 2H), 4.14 (s, |
| | | | | 1H), 3.83-3.87 (m, 1H), 3.42-3.45 (m, 1H), 3.03-3.08 (m, 1H), |
| | | | | 2.63-2.69 (m, 1H). |
| 166 | 431.3 | L3 | 4.59 | (DMSO-d₆) δ ppm: 8.26 (d, 1H), 7.61 (d, 2H), 7.34 (d, 2H), |
| | | | | 7.268 (d, 1H), 7.11 (t, 1H), 7.04 (d, 1H), 6.16-6.22 (m, 1H), 6.02 |
| | | | | (dd, 1H), 5.53 (dd, 1H), 4.08-4.17 (m, 1H), 3.85-3.89 (m, 1H), |
| | | | | 3.37-3.43 (m, 2H), 2.89-2.96 (m, 1H). |
| 166-En1 | 431.3 | S8 | 1.75 | (DMSO-d₆) δ ppm: 12.8 (s, 1H), 8.26 (d, 1H), 7.66 (d, 2H), 7.38 |
| | | | | (d, 2H), 7.20-7.24 (m, 1H), 7.14-7.17 (m, 2H), 6.15-6.22 (m, |
| | | | | 1H), 6.02 (dd, 1H), 5.54 (dd, 1H), 4.18-4.21 (m, 1H), 3.84-3.88 |
| | | | | (m, 1H), 3.46-3.51 (m, 1H), 3.24-3.28 (m, 1H), 2.89-2.95 (m, |
| | | | | 1H). |
| 166-En2 | 431.3 | S8 | 3.45 | (DMSO-d₆) δ ppm: 12.70 (s, 1H), 8.26 (d, 1H), 7.65 (d, 2H), |
| | | | | 7.38 (d, 2H), 7.11-7.22 (m, 3H), 6.15-6.22 (m, 1H), 6.03 (dd, |
| | | | | 1H), 5.54 (m, 1H), 4.16-4.18 (m, 1H), 3.85-3.88 (m, 1H), 3.45- |
| | | | | 3.50 (m, 1H), 3.25-3.31 (m, 1H), 2.89-2.95 (m, 1H). |
| 164 | 386.1 | L3 | 3.31 | (DMSO-d₆) δ ppm: 8.29 (d, 1H), 7.62 (d, 2H), 7.35 (d, 2H), 7.09 |
| | | | | (t, 1H), 6.96 (t, 2H), 6.16-6.23 (m, 1H), 6.03 (dd, 1H), 5.55 (dd, |
| | | | | 1H), 4.19-4.23 (m, 1H), 3.98 (s, 2H), 3.80-3.84 (m, 1H), 3.44- |
| | | | | 3.49 (m, 1H), 3.05-3.11 (m, 1H), 2.65-2.71 (m, 1H). |
| 162 | 405.3 | L2 | 2.75 | (DMSO-d₆) δ ppm: 12.65 (br s, 1H), 8.41 (d, 1H), 7.59 (d, 2H), |
| | | | | 7.31 (d, 2H), 7.00 (t, 1H), 6.90 (d, 1H), 6.82 (d, 1H), 6.16-6.23 |
| | | | | (m, 1H), 6.03 (dd, 1H), 5.54 (dd, 1H), 4.15-4.22 (m, 1H), 3.82- |
| | | | | 3.86 (m, 1H), 3.48-3.60 (m, 2H), 3.37-3.42 (m, 1H), 2.94-3.00 |
| | | | | (m, 1H), 2.65-2.71 (m, 1H). |
| 162-En1 | 405.3 | S5 | 1.21 | (DMSO-d₆) δ ppm: 8.55 (br s, 1H), 7.58 (d, 2H), 7.29 (d, 2H), |
| | | | | 6.99 (t, 1H), 6.90 (d, 1H), 6.80 (d, 1H), 6.16-6.23 (m, 1H), 6.03 |
| | | | | (dd, 1H), 5.53 (dd, 1H), 4.17-4.26 (m, 1H), 3.81-3.85 (m, 1H), |
| | | | | 3.52-3.56 (m, 1H), 3.38-3.45 (m, 1H), 2.90-2.95 (m, 1H), 2.66- |
| | | | | 2.75 (m, 1H). |
| 162-En2 | 405.3 | S5 | 2.06 | (DMSO-d₆) δ ppm: 8.55 (br s, 1H), 7.58 (d, 2H), 7.29 (d, 2H), |
| | | | | 6.99 (t, 1H), 6.90 (d, 1H), 6.81 (d, 1H), 6.16-6.23 (m, 1H), 6.03 |
| | | | | (dd, 1H), 5.53 (dd, 1H), 4.17-4.26 (m, 1H), 3.81-3.85 (m, 1H), |
| | | | | 3.54-3.58 (m, 1H), 3.38-3.48 (m, 1H), 2.91-2.96 (m, 1H), 2.67- |
| | | | | 2.73 (m, 1H). |
| 160 | 468.3 | L1 | 2.78 | (DMSO-d₆) δ ppm: 12.5 (br s, 1H), 8.27 (d, 1H), 7.61 (d, 2H), |
| | | | | 7.32 (d, 2H), 7.01-7.08 (m, 3H), 6.17-6.24 (m, 1H), 6.03 (dd, |
| | | | | 1H), 5.54 (dd, 1H), 4.09-4.18 (m, 1H), 3.84-3.88 (m, 1H), 3.37- |
| | | | | 3.42 (m, 1H), 3.15-3.23 (m, 4H), 2.87-2.94 (m, 1H). |
| 160-En1 | 468.3 | S8 | 1.92 | (DMSO-d₆) δ ppm: 12.16 (s, 1H), 8.28 (d, 1H), 7.65 (d, 2H), |
| | | | | 7.35 (d, 2H), 7.04-7.16 (m, 3H), 6.16-6.23 (m, 1H), 6.05 (dd, |
| | | | | 1H), 5.56 (dd, 1H), 4.14-4.18 (m, 1H), 3.84-3.88 (m, 1H), 3.41- |
| | | | | 3.46 (m, 1H), 3.35 (s, 3H), 3.11-3.17 (m, 1H), 2.87-2.94 (m, |
| | | | | 1H). |
| 160-En2 | 468.3 | S8 | 2.52 | (DMSO-d₆) δ ppm: 12.16 (s, 1H), 8.28 (d, 1H), 7.65 (d, 2H), |
| | | | | 7.35 (d, 2H), 7.04-7.16 (m, 3H), 6.16-6.23 (m, 1H), 6.04 (dd, |
| | | | | 1H), 5.56 (dd, 1H), 4.14-4.18 (m, 1H), 3.84-3.88 (m, 1H), 3.41- |
| | | | | 3.46 (m, 1H), 3.35 (s, 3H), 3.11-3.17 (m, 1H), 2.87-2.94 (m, |
| | | | | 1H). |
| 169 | 415.3 | L2 | 2.65 | (DMSO-d₆) δ ppm: 8.239 (d, 1H), 7.57 (d, 1H), 7.29 (d, 2H), |
| | | | | 6.93-7.20 (m, 6H), 6.16-6.22 (m, 1H), 6.02 (dd, 1H), 5.53 (dd, |
| | | | | 1H), 4.02-4.09 (m, 1H), 3.84-3.88 (m, 1H), 3.25-3.38 (m, 2H), |
| | | | | 2.87-2.93 (m, 1H). |
| 165 | 482.3 | L2 | 2.74 | (DMSO-d₆) δ ppm: 11.99 (s, 1H), 8.25 (d, 1H), 7.60 (d, 2H), |
| | | | | 7.33 (d, 2H), 7.02 (t, 1H), 6.91 (d, 1H), 6.83 (s, 1H), 6.16-6.23 |
| | | | | (m, 1H), 6.03 (dd, 1H), 5.55 (dd, 1H), 4.15-4.24 (m, 1H), 3.81- |
| | | | | 3.84 (m, 1H), 3.64 (s, 2H), 3.40-3.45 (m, 1H), 3.26 (s, 3H), |
| | | | | 2.98-3.03 (m, 1H), 2.60-2.66 (m, 1H). |
| 080 | 348.2 | L2 | 2.02 | (DMSO-d₆) δ ppm: 8.28-8.29 (d, 1H), 8.05-8.06 (m, 1H), 7.64- |
| | | | | 7.67 (d, 2H), 7.37-7.39 (d, 2H), 7.29-7.31 (m, 1H), 7.04-7.07 |
| | | | | (m, 1H), 6.16-6.22 (m, 1H), 6.00-6.05 (dd, 1H), 5.52-5.55 (dd, |
| | | | | 1H), 4.26-4.31 (m, 1H), 3.81-3.85 (dd, 1H), 3.54-3.59 (dd, 1H), |
| | | | | 3.19-3.25 (dd, 1H), 2.88-2.94 (dd, 1H). |
| 081 | 348.2 | L2 | 1.98 | (DMSO-d₆) δ ppm: 8.01-8.03 (dd, 1H), 7.60-7.62 (d, 2H), 7.41- |
| | | | | 7.43 (d, 2H), 7.34-7.36 (m, 1H), 6.73-6.76 (m, 1H), 6.26-6.30 |
| | | | | (dd, 1H), 6.00-6.06 (m, 1H), 5.74-5.75 (d, 1H), 5.64-5.67 (dd, |
| | | | | 1H), 4.65-4.68 (m, 1H), 3.96-4.00 (m, 1H), 3.80-3.85 (m, 1H), |
| | | | | 3.22-3.26 (m, 1H), 2.88-2.93 (m, 1H). |
| 082 | 348.2 | L2 | 1.82 | (DMSO-d₆) δ ppm: 8.28 (d, 1H), 8.14 (s, 1H), 7.97 (d, 1H), 7.77 |
| | | | | (d, 2H), 7.51 (d, 2H), 6.59 (d, 1H), 6.22-6.29 (m, 1H), 6.07 (dd, |
| | | | | 1H), 5.57 (dd, 1H), 4.29-4.33 (d, 1H), 3.79-3.83 (m, 1H), 3.55- |
| | | | | 3.59 (m, 1H), 3.07-3.12 (m, 1H), 2.76-2.81 (m, 1H). |
| 083 | 348.2 | L2 | 1.92 | (DMSO-d₆) δ ppm: 8.21 (s, 1H), 8.04-8.05 (d, 1H), 7.60-7.62 |
| | | | | (d, 2H), 7.30-7.32 (d, 2H), 7.00-7.01 (d, 1H), 6.22-6.27 (dd, |
| | | | | 1H), 5.97-5.04 (m, 1H), 5.72-5.74 (d, 1H), 5.62-5.65 (dd, 1H), |
| | | | | 4.63-4.67 (m, 1H), 3.82-3.86 (m, 1H), 3.69-3.73 (m, 1H), 3.17- |
| | | | | 3.23 (m, 1H), 2.84-2.90 (m, 1H). |
| 140 | 363.3 | L2 | 2.10 | (DMSO d₆) δ ppm: 8.15 (d, 1H), 7.49 (d, 2H), 7.11-7.17 (m, |
| | | | | 3H), 6.29 (d, 1H), 5.95-6.14 (m, 2H), 5.76 (s, 2H), 5.48-5.51 |
| | | | | (m, 1H), 4.08 (t, 1H), 3.83-3.87 (dd, 1H), 3.40-3.45 (m, 1H), |
| | | | | 2.92-2.98 (m, 1H), 2.61-2.68 (m, 1H). |
| 146 | 363.3 | L2 | 2.08 | (DMSO-d₆) δ ppm: 8.28 (d, 1H), 7.68 (d, 2H), 7.50 (d, 1H), 7.41 |
| | | | | (d, 2H), 6.20-6.27 (m, 1H), 6.00-6.05 (m, 2H), 5.52-5.58 (m, |
| | | | | 3H), 4.28-4.30 (m, 1H), 3.67-3.71 (m, 1H), 3.49-3.54 (m, 1H), |
| | | | | 2.74-2.79 (m, 1H), 2.34-2.40 (m, 1H). |
| 141 | 378.2 | L3 | 5.08 | (DMSO d₆) δ ppm: 7.51 (d, 2H), 7.31 (d, 1H), 7.15 (d, 2H), |
| | | | | 6.521 (d, 1H), 6.12-6.17 (m, 1H), 5.88-5.95 (m, 1H), 5.54-5.58 |
| | | | | (m, 2H), 4.59-4.61 (m, 1H), 3.90 (s, 3H), 3.78-3.79 (m, 2H), |
| | | | | 3.24-3.30 (m, 1H), 2.85-2.91 (m, 1H). |
| 084 | 361.3 | L2 | 3.10 | (CDCl₃) δ ppm: 8.20-8.21 (t, 1H), 7.61-7.63 (d, 2H), 7.33-7.34 |
| | | | | (d, 2H), 7.12-7.14 (d, 1H), 6.94-7.03 (m, 2H), 6.81-6.85 (m, |
| | | | | 1H), 6.18-6.25 (m, 1H), 6.04-6.09 (dd, 1H), 5.56-5.59 (dd, 1H), |
| | | | | 3.78-3.82 (m, 1H), 3.25-3.31 (m, 1H), 3.17-3.20 (t, 2H), 2.82- |
| | | | | 2.87 (m, 1H), 2.52-2.55 (m, 1H), 2.11-2.18 (m, 1H). |
| 084-En1 | 361.3 | S3 | 3.04 | (CDCl₃) δ ppm: 8.20-8.23 (t, 1H), 7.62-7.64 (d, 2H), 7.32-7.34 |
| | | | | (d, 2H), 7.12-7.14 (d, 1H), 6.94-7.03 (m, 2H), 6.81-6.85 (m, |
| | | | | 1H), 6.18-6.25 (m, 1H), 6.04-6.09 (dd, 1H), 5.56-5.59 (dd, 1H), |
| | | | | 3.79-3.82 (m, 1H), 3.25-3.32 (m, 1H), 3.17-3.21 (t, 2H), 2.82- |
| | | | | 2.87 (m, 1H), 2.52-2.55 (m, 1H), 2.12-2.17 (m, 1H). |
| 084-En2 | 361.3 | S3 | 3.04 | (CDCl₃) δ ppm: 8.20-8.23 (t, 1H), 7.62-7.64 (d, 2H), 7.32-7.34 |
| | | | | (d, 2H), 7.12-7.14 (d, 1H), 6.94-7.03 (m, 2H), 6.81-6.85 (m, |
| | | | | 1H), 6.18-6.25 (m, 1H), 6.04-6.09 (dd, 1H), 5.56-5.59 (dd, 1H), |
| | | | | 3.79-3.82 (m, 1H), 3.25-3.32 (m, 1H), 3.17-3.21 (t, 2H), 2.82- |
| | | | | 2.87 (m, 1H), 2.52-2.55 (m, 1H), 2.11-2.18 (m, 1H). |
| 085 | 293.2 | L2 | 2.85 | (CDCl₃) δ ppm 7.33-7.37 (m, 2H), 7.20-7.23 (m, 2H), 7.11-7.13 |
| | | | | (m, 1H), 7.04-7.09 (m, 1H), 6.91-6.95 (m, 1H), 6.69-6.73 (m, |
| | | | | 2H), 6.20-6.25 (dd, 1H), 5.98-6.02 (m, 1H), 5.61-5.64 (dd, 1H), |
| | | | | 5.60-5.54 (br s, 1H), 3.72-3.68 (m, 1H), 3.34-3.49 (m, 3H), |
| | | | | 2.95-3.01 (m, 1H), 2.62-2.68 (m, 1H), 2.38-2.43 (m, 1H). |
| 086 | 361.2 | L2 | 2.99 | (DMSO-d6) δ ppm 8.20-8.23 (t, 1H), 7.54-7.58 (m, 1H), 7.47- |
| | | | | 7.50 (m, 2H), 7.37-7.38 (d, 1H), 7.08-7.10 (d, 1H), 6.94-6.98 |
| | | | | (m, 1H), 6.71-6.78 (m, 2H), 6.18-6.25 (m, 1H), 6.04-6.09 (dd, |
| | | | | 1H), 5.55-5.58 (dd, 1H), 3.72-3.75 (m, 1H), 3.29-3.31 (m, 1H), |
| | | | | 3.18-3.21 (m, 2H), 2.83-2.89 (m, 1H), 2.53-2.57 (m, 1H), 2.16- |
| | | | | 2.19 (m, 1H). |
| 087 | 335.3 | L2 | 3.17 | (DMSO-d6) δ ppm 8.1-8.3 (t, 1H), 7.21-7.26 (d, 2H), 7.11-7. |
| | | | | 14 (d, 2H), 6.99-7.00 (d, 1H), 6.83-6.87 (m, 1H), 6.58-6.62 (m, |
| | | | | 1H), 6.46-6.48 (m, 1H), 6.19-6.25 (m, 1H), 6.04-6.09 (dd, 1H), |
| | | | | 5.55-5.58 (dd, 1H), 3.57-3.60 (m, 1H), 3.17-3.26 (m, 3H), 2.81- |
| | | | | 2.91 (m, 2H), 2.54-2.58 (m, 1H), 2.16-2.18 (m, 1H), 1.20-1.21 |
| | | | | (d, 6H). |
| 088 | 335.3 | L2 | 3.18 | (DMSO-d₆) δ ppm 8.20-8.24 (t, 1H), 7.26-7.31 (m, 1H), 7.07- |
| | | | | 7.09 (m, 1H), 7.00-7.03 (m, 3H), 6.84-6.89 (m, 1H), 6.60-6.65 |
| | | | | (m, 1H), 6.51-6.54 (m, 1H), 6.19-6.26 (m, 1H), 6.04-6.10 (dd, |
| | | | | 1H), 5.55-5.59 (dd, 1H), 3.60-3.65 (m, 1H), 3.24-3.29 (m, 1H), |
| | | | | 3.18-3.22 (m, 2H), 2.82-2.89 (m, 2H), 2.54-2.59 (m, 1H), 2.14- |
| | | | | 2.23 (m, 1H), 1.19 (d, 6H). |
| 089 | 327.2 | L2 | 3.04 | (DMSO-d₆) δ ppm: 8.19-8.22 (t, 1H), 7.37-7.41 (m, 2H), 7.19- |
| | | | | 7.23 (m, 2H), 7.05 (d, 1H), 6.89-6.93 (t, 1H), 6.69-6.71 (t, 1H), |
| | | | | 6.61-6.63 (d, 1H), 6.18-6.25 (m, 1H), 6.04-6.09 (dd, 1H), 5.59 |
| | | | | (dd, 1H), 3.61-3.65 (dd, 1H), 3.17-3.27 (m, 3H, 2.82-2.87 (dd, |
| | | | | 1H), 2.51-2.57 (m, 1H), 2.15-2.17 (m, 1H). |
| 090 | 323.3 | L6 | 2.58 | (DMSO-d₆) δ ppm 8.1-8.3 (t, 1H), 7.24-7.28 (t, 1H), 7.01-7.03 |
| | | | | (m, 1H), 6.89 (m, 1H), 6.73-6.78 (m, 2H), 6.61-6.70 (m, 3H), |
| | | | | 6.19-6.26 (m, 1H), 6.04-6.09 (dd, 1H), 5.56-5.59 (dd, 1H), 3.72 |
| | | | | (s, 3H), 3.62-3.66 (m, 1H), 3.17-3.27 (m, 1H), 2.82-2.87 (m, |
| | | | | 1H), 2.49-2.56 (m, 1H), 2.15-2.17 (m, 1H). |
| 091 | 323.3 | L2 | 2.83 | (DMSO-d6) δ ppm 8.15-8.25 (t, 1H), 7.12-7.16 (m, 2H), 6.95- |
| | | | | 6.99 (m, 3H), 6.80-6.84 (m, 1H), 6.53-6.57 (m, 1H), 6.27-6.29 |
| | | | | (m, 1H), 6.19-6.26 (m, 1H), 6.04-6.09 (dd, 1H), 5.55-5.58 (dd, |
| | | | | 1H), 3.76 (s, 3H), 3.49-3.52 (m, 1H), 3.18-3.26 (m, 3H), 2.82- |
| | | | | 2.87 (m, 1H), 2.52-2.59 (m, 1H), 2.18-2.21 (m, 1H). |
| 092 | 369.3 | L2 | 3.25 | (DMSO-d₆) δ ppm: 8.23 (t, 1H), 7.65-7.67 (m, 4H), 7.43-7.47 |
| | | | | (t, 2H), 7.28-7.35 (m, 3H), 7.05-7.06 (d, 1H), 6.91-6.95(t, 1H), |
| | | | | 6.67-6.73(m, 2H), 6.19-6.26 (m, 1H), 6.05-6.10 (m, 1H), 5.56- |
| | | | | 5.59 (dd, 1H), 3.69-3.73 (dd, 1H), 3.30 (m, 1H), 3.17-3.27(m, |
| | | | | 2H), 2.84-2.89 (dd, 1H) 2.55-2.58 (m, 1H), 2.18-2.20 (m, 1H). |
| 093 | 369.3 | L2 | 3.17 | (DMSO-d₆) δ ppm: 8.20-8.25 (t, 1H), 7.62-7.66 (m, 2H), 7.44- |
| | | | | 7.49 (m, 4H), 7.35-7.41 (m, 2H), 7.20-7.23 (m, 1H), 7.03-7.07 |
| | | | | (m, 1H), 6.88-6.94 (m, 1H), 6.64-6.70 (m, 2H), 6.19-6.26 (m, |
| | | | | 1H), 6.04-6.10 (dd, 1H), 5.54-5.59 (dd, 1H), 3.71-3.76 (m, 1H), |
| | | | | 3.33-3.38 (m, 1H), 3.20-3.26 (m, 2H), 2.84-2.92 (m, 1H), 2.53- |
| | | | | 2.61 (m, 1H), 2.16-2.26 (m, 1H). |
| 094 | 362.2 | L2 | 2.71 | (DMSO-d₆) δ ppm: 8.58-8.61 (s, 1H), 8.18-8.23 (t, 1H), 7.71- |
| | | | | 7.79 (m, 2H), 7.15-7.20 (m, 1H), 7.02-7.08 (m, 2H), 6.86-6.92 |
| | | | | (m, 1H), 6.18-6.26 (m, 1H), 6.03-6.10 (dd, 1H), 5.54-5.61 (dd, |
| | | | | 1H), 3.83-3.90 (m, 1H), 3.33-3.38 (m, 1H), 3.17-3.22 (m, 2H), |
| | | | | 2.83-2.91 (m, 1H), 2.51-2.58 (m, 1H), 2.13-2.24 (m, 1H). |
| 095 | 362.2 | L2 | 2.81 | (DMSO-d₆) δ ppm: 8.55-8.57 (s, 1H), 8.22-8.26 (t, 1H), 7.81- |
| | | | | 7.86 (m, 1H), 7.34-7.39 (m, 1H), 7.21-7.26 (m, 2H), 7.13-7.19 |
| | | | | (m, 1H), 7.01-7.08 (m, 1H), 6.18-6.28 (m, 1H), 6.03-6.10 (dd, |
| | | | | 1H), 5.56-5.62 (dd, 1H), 4.24-4.30 (m, 1H), 3.36-3.43 (m, 1H), |
| | | | | 3.15-3.21 (m, 2H), 2.83-2.90 (m, 1H), 2.46-2.53 (m, 1H), 2.08- |
| | | | | 2.18 (m, 1H). |
| 096 | 362.2 | L2 | 2.54 | (DMSO-d₆) δ ppm: 8.38-8.41 (s, 1H), 8.22-8.26 (t, 1H), 7.40- |
| | | | | 7.43 (m, 1H), 7.25-7.30 (m, 3H), 7.15-7.20 (m, 1H), 7.03-7.06 |
| | | | | (m, 1H), 6.18-6.27 (m, 1H), 6.04-6.10 (dd, 1H), 5.57-5.61 (dd, |
| | | | | 1H), 3.91-3.98 (m, 1H), 3.27-3.30 (m, 1H), 3.18-3.23 (m, 2H), |
| | | | | 2.79-2.86 (m, 1H), 2.44-2.47 (m, 1H), 2.10-2.17 (m, 1H). |
| 097 | 311.3 | L1 | 2.40 | (CDCl₃) δ ppm: 7.16-7.20 (m, 2H), 7.03-7.08 (m, 3H), 6.90-6.94 |
| | | | | (m, 1H), 6.67-6.71 (m, 1H), 6.52-6.54 (d, 1H), 6.24-6.28 (dd, |
| | | | | 1H), 6.02-6.09 (m, 1H), 5.63-5.66 (m, 2H), 3.59-3.63 (m, 1H), |
| | | | | 3.34-3.48 (m, 3H), 2.95-3.00 (m, 1H), 2.63-2.69 (m, 1H), 2.38- |
| | | | | 2.46 (m, 1H). |
| 098 | 363.3 | L2 | 3.07 | (DMSO-d₆) δ ppm: 7.87-7.85 (t, 1H), 7.63-7.61 (d, 2H), 7.33- |
| | | | | 7.31 (d, 2H), 7.13-7.11 (d, 1H), 7.02-6.94 (m, 2H), 6.85-6.81 (t, |
| | | | | 1H), 3.80-3.76 (dd, 1H), 3.27-3.22 (t, 1H), 3.10-3.07 (d, 2H), |
| | | | | 2.86-2.81 (dd, 1H), 2.51 (m, 1H), 2.13-2.04 (m, 3H), 0.98-0.95 |
| | | | | (t, 3H). |
| 099 | 389.3 | L3 | 5.75 | (DMSO-d₆) δ ppm: 7.85-7.87 (t, 1H), 7.61-7.63 (d, 2H), 7.31- |
| | | | | 7.33 (d, 2H), 7.11-7.13 (d, 1H), 6.94-7.02 (m, 2H), 6.80-6.84 |
| | | | | (m, 1H), 5.63-5.64 (s, 1H), 3.77-3.81 (m, 1H), 3.13-3.31 (m, |
| | | | | 1H), 3.09-3.12 (m, 2H), 2.80-2.85 (m, 1H), 2.46-2.52 (m, 1H), |
| | | | | 2.04-2.12 (m, 4H), 1.76 (s, 3H). |
| 100 | 350.3 | L2 | 2.30 | (DMSO-d₆) δ ppm: 8.26-8.33 (q, 1H), 8.19-8.26 (t, 1H), 7.78- |
| | | | | 7.80 (d, 2H), 7.21-7.23 (d, 2H), 7.08-7.10 (d, 1H), 6.94-6.98 (m, |
| | | | | 1H), 6.83-6.85 (m, 1H), 6.74-6.78 (m, 1H), 6.18-6.25 (m, 1H), |
| | | | | 6.04-6.09 (dd, 1H), 5.58-5.59 (dd, 1H), 3.74 (m, 1H), 3.19-3.31 |
| | | | | (m, 1H), 3.15-3.17 (m, 2H), 2.82-2.87 (m, 1H), 2.67-2.77 (d, |
| | | | | 3H), 2.49-2.55 (m, 1H), 2.13-2.17 (m, 1H). |
| 101 | 329.3 | L1 | 2.45 | (CDCl₃) δ ppm: 7.09-7.13 (m, 1H), 7.04-7.06 (m, 2H), 7.00-7.03 |
| | | | | (m, 2H), 6.91-6.96 (m, 1H), 6.65-6.76 (d, 1H), 6.25-6.29 (dd, |
| | | | | 1H), 6.03-6.09 (m, 1H), 5.64-5.67 (dd, 1H), 5.58-5.63 (t, 1H), |
| | | | | 3.60-3.64 (m, 1H), 3.31-3.47 (m, 3H), 2.93-2.98 (m, 1H), 2.60- |
| | | | | 2.67 (m, 1H), 2.35-2.41 (m, 1H). |
| 102 | 329.3 | L2 | 2.39 | (DMSO-d₆) δ ppm: 8.18-8.26 (t, 1H), 7.36-7.47 (m, 2H), 7.12- |
| | | | | 7.20 (m, 1H), 7.00-7.01 (m, 1H), 6.84-6.89 (m, 1H), 6.60-6.64 |
| | | | | (m, 1H), 6.19-6.26 (m, 1H), 6.05-6.12 (m, 2H), 5.56-5.59 (dd, |
| | | | | 1H), 3.46-3.50 (m, 1H), 3.20-3.29 (m, 3H), 2.84-2.90 (m, 1H), |
| | | | | 2.55-2.62 (m, 1H), 2.21-2.23 (t, 1H). |
| 103 | 332.3 | L2 | 2.68 | (DMSO-d₆) δ ppm: 11.12 (s, 1H), 8.19-8.23 (t, 1H), 7.41-7.43 |
| | | | | (d, 1H), 7.35-7.36 (m, 2H), 6.92-6.96 (m, 2H), 6.74-6.78 (m, |
| | | | | 1H), 6.49-6.53 (m, 1H), 6.39-6.40 (s, 1H), 6.19-6.26 (m, 2H), |
| | | | | 6.04-6.09 (dd, 1H), 5.54-5.57 (dd, 1H), 3.54-3.58 (m, 1H), 3.28- |
| | | | | 3.33 (m, 1H), 3.21-3.26 (t, 2H), 2.84-2.89 (m, 1H), 2.56-2.62 |
| | | | | (m, 1H), 2.07-2.24 (m, 1H). |
| 104 | 332.3 | L2 | 2.72 | (DMSO-d₆) δ ppm: 11.03 (s, 1H), 8.19-8.29 (t, 1H), 7.53-7.55 |
| | | | | (d, 1H), 7.32-7.33 (t, 1H), 7.20 (s, 1H), 6.97-6.99 (d, 1H), 6.84- |
| | | | | 6.87 (dd, 1H), 6.79 (m, 1H), 6.54-6.55 (m, 1H), 6.40-6.41 (m, |
| | | | | 1H), 6.33-6.35 (m, 1H), 6.19-6.25 (m, 1H), 6.04-6.09 (dd, 1H), |
| | | | | 5.54-5.58 (dd, 1H), 3.58-3.62 (m, 1H), 3.34 (m, 1H), 3.21-3.24 |
| | | | | (m, 2H), 2.84-2.89 (m, 1H), 2.56-2.62 (m, 1H), 2.22-2.24 (m, |
| | | | | 1H). |
| 105 | 343.2 | L2 | 2.82 | (CDCl₃) δ ppm: 7.44-7.51 (d, 2H), 7.26-7.28 (d, 2H), 7.08-7.09 |
| | | | | (d, 1H), 6.96-7.00 (m, 1H), 6.88-6.90 (d, 1H), 6.78-6.82 (m, |
| | | | | 1H), 6.47-6.78 (m, 1H), 6.22-6.26 (dd, 1H), 5.99-6.06 (m, 1H), |
| | | | | 5.62-5.65 (dd, 1H), 5.52-5.59 (t, 1H), 3.71-3.75 (m, 1H), 3.33- |
| | | | | 3.45 (m, 3H), 2.94-2.99 (m, 1H), 2.60-2.66 (m, 1H), 2.36-2.43 |
| | | | | (m 1H). |
| 106 | 377.2 | L2 | 3.09 | (CDCl₃) δ ppm: 7.17-7.22 (m, 4H), 7.05-7.07 (d, 1H), 6.93-6.98 |
| | | | | (m, 1H), 6.71-6.77 (m, 2H), 6.23-6.27 (dd, 1H), 6.01-6.08 (m, |
| | | | | 1H), 5.63-5.68 (dd, 1H), 5.56-5.62 (t, 1H), 3.64-3.68 (m, 1H), |
| | | | | 3.35-3.42 (m, 3H), 2.94-2.99 (m, 1H), 2.61-2.67 (m, 1H), 2.36- |
| | | | | 2.43 (m, 1H). |
| 107 | 359.2 | L2 | 2.85 | (CDCl₃) δ ppm: 7.19-7.22 (m, 2H), 7.10-7.13 (m, 2H), 7.04-7.06 |
| | | | | (d, 1H), 6.92-6.94 (t, 1H), 6.72-6.74 (m, 1H), 6.32-6.71 (m, 2H), |
| | | | | 6.23-6.28 (dd, 1H), 6.02-6.08 (m, 1H), 5.63-5.66 (dd, 1H), 5.58- |
| | | | | 5.63 (t, 1H), 3.62-3.66 (m, 1H), 3.35-3.43 (m, 3H), 2.95-3.00 |
| | | | | (m, 1H), 2.62-2.68 (m, 1H), 2.37-2.43 (m, 1H). |
| 108 | 311.2 | L4 | 2.75 | (CDCl₃) δ ppm: 7.23-7.27 (m, 1H), 7.06-7.08 (d, 1H), 6.96-6.99 |
| | | | | (m, 2H), 6.89-6.93 (m, 1H), 6.85-6.87 (d, 1H), 6.75-6.80 (m, |
| | | | | 1H), 6.22-6.27 (dd, 1H), 6.00-6.07 (m, 1H), 5.63-5.66 (dd, 1H), |
| | | | | 5.54-5.61 (t, 1H), 3.63-3.73 (m, 1H), 3.32-3.45 (m, 3H), 2.93- |
| | | | | 2.98 (m, 1H), 2.59-2.65 (m, 1H), 2.36-2.42 (m, 1H). |
| 109 | 329.2 | L2 | 2.73 | (CDCl₃) δ ppm: 7.04-7.09 (m, 4H), 6.93-6.97 (m, 1H), 6.71-6.75 |
| | | | | (m, 1H), 6.41-6.43 (d, 1H), 6.23-6.28 (dd, 1H), 6.03-6.10 (m, |
| | | | | 1H), 5.71-5.79 (t, 1H), 5.63-5.66 (dd, 1H), 3.60-3.64 (m, 1H), |
| | | | | 3.37-3.46 (m, 3H), 3.00-3.05 (m, 1H), 2.66-2.72 (m, 1H), 2.43- |
| | | | | 2.49 (m, 1H). |
| 110 | 329.2 | L2 | 2.73 | (CDCl3) δ ppm: 7.02-7.09 (m, 4H), 6.94-6.96 (m, 1H), 6.71- |
| | | | | 6.75 (m, 1H), 6.40-6.43 (d, 1H), 6.27-6.28 (dd, 1H), 6.03-6.23 |
| | | | | (m, 1H), 5.65-5.657 (d, 1H), 5.62-5.63 (dd, 1H), 3.60-3.63 (dd, |
| | | | | 1H), 3.37-3.46 (m, 3H), 2.98-3.05 (dd, 1H), 2.65-2.71 (dd, 1H), |
| | | | | 2.43-2.49 (m, 1H). |
| 111 | 329.2 | L2 | 2.82 | (CDCl₃) δ ppm: 7.08-7.10 (d, 1H), 6.99-7.03 (m, 2H), 6.83-6.87 |
| | | | | (m, 1H), 6.68-6.70 (m, 2H), 6.42-6.46 (m, 1H), 6.25-6.29 (dd, |
| | | | | 1H), 6.02-6.09 (m, 1H), 5.65-5.67 (dd, 1H), 5.56-5.62 (t, 1H), |
| | | | | 3.68-3.71 (m, 1H), 3.31-3.42 (m, 3H), 2.90-2.96 (m, 1H), 2.56- |
| | | | | 2.62 (m, 1H), 2.34-2.38 (m, 1H). |
| 112 | 379.2 | L2 | 3.04 | (CDCl₃) δ ppm: 7.43-7.48 (m, 1H), 7.04-7.15 (m, 3H), 6.89-7.01 |
| | | | | (m, 3H), 6.25-6.30 (dd, 1H), 6.02-6.09 (m, 1H), 5.65-5.68 (dd, |
| | | | | 1H), 5.56-5.63 (t, 1H), 3.75-3.78 (m, 1H), 3.33-3.39 (m, 3H), |
| | | | | 2.90-2.95 (m, 1H), 2.56-2.62 (m, 1H), 2.33-2.39 (m, 1H). |
| 113 | 379.2 | L4 | 2.94 | (CDCl₃) δ ppm: 7.40-7.43 (m, 3H), 7.06-7.08 (d, 1H), 6.94-6.98 |
| | | | | (m, 1H), 6.75-6.79 (m, 1H), 6.47-6.49 (d, 1H), 6.23-6.28 (dd, |
| | | | | 1H), 6.02-6.09 (m, 1H), 5.68-5.73 (t, 1H), 5.63-5.66 (dd, 1H), |
| | | | | 3.62-3.66 (m, 1H), 3.38-3.45 (m, 3H), 2.99-3.05 (m, 1H), 2.65- |
| | | | | 2.71 (m, 1H), 2.41-2.47 (m, 1H). |
| 114 | 393.2 | L4 | 3.15 | (CDCl₃) δ ppm: 7.53-7.58 (d, 2H), 7.20-7.23 (d, 2H), 7.09-7.11 |
| | | | | 9d, 1H), 6.99-7.01 (m, 2H), 6.82-6.86 (m, 1H), 6.22-6.26 (dd, |
| | | | | 1H), 5.99-6.06 (m, 1H), 5.63-5.66 (dd, 1H), 5.51-5.58 (t, 1H), |
| | | | | 3.73-3.77 (m, 1H), 3.31-3.44 (m, 3H), 2.92-2.97 (m, 1H), 2.58- |
| | | | | 2.64 (m, 1H), 2.35-241 (m, 1H). |
| 115 | 363.2 | L2 | 2.56 | (CDCl₃) δ ppm: 8.70 (s, 2H), 7.17-7.18 (m, 1H), 7.08-7.10 (m, |
| | | | | 2H), 6.97-7.01 (m, 1H), 6.28-6.32 (dd, 1H), 6.04-6.08 (m, 1H), |
| | | | | 5.62-5.70 (m, 2H), 3.81-3.86 (m, 1H), 3.33-3.50 (m, 3H), 2.92- |
| | | | | 2.97 (m, 1H), 2.59-2.65 (m, 1H), 2.37-2.42 (m, 1H). |
| 116 | 321.3 | L2 | 3.09 | (DMSO-d6) δ ppm: 8.19-8.25 (t, 1H), 7.14 (s, 1H), 7.02-7.09 |
| | | | | (m, 2H), 6.95-6.99 (d, 1H), 6.77-6.81 (m, 1H), 6.49-6.52 (m, |
| | | | | 1H), 6.20-6.27 (m, 1H), 6.05-6.10 (dd, 1H), 5.82-5.84 (br d, |
| | | | | 1H), 5.56-5.59 (dd, 1H), 3.47 (br s, 1H), 3.18-3.27 (m, 3H), |
| | | | | 2.82-2.87 (m, 1H), 2.55-2.60 (m, 1H), 2.29 (s, 3H), 2.22-2.24 |
| | | | | (m, 1H), 2.05 (s, 3H). |
| 117 | 419.2 | L4 | 3.06 | (CDCl₃) δ ppm: 7.62-7.66 (d, 2H), 7.19-7.20 (d, 2H), 7.11-7.12 |
| | | | | (d, 1H), 7.02-7.04 (m, 2H), 6.85-6.89 (m, 1H), 6.23-6.28 (dd, |
| | | | | 1H), 6.01-6.08 (d, 1H), 5.64-5.67 (dd, 1H), 5.53-5.60 (t, 1H), |
| | | | | 3.75-3.79 (m, 1H), 3.36-3.41 (m, 3H), 2.91-2.97 (m, 1H), 2.57- |
| | | | | 2.64 (m, 1H), 2.33-2.40 (m, 1H). |
| 147 | 318.2 | L2 | 2.73 | (DMSO d₆) δ ppm: 8.21 (t, 1H), 7.68 (d, 2H), 7.26 (d, 2H), 7.16 |
| | | | | (d, 1H), 7.03-7.06 (m, 2H), 6.88-6.92 (m, 1H), 6.18-6.24 (m, |
| | | | | 1H), 6.06 (dd, 1H), 5.58 (dd, 1H), 3.82-3.86 (m, 1H), 3.23-3.31 |
| | | | | (m, 1H), 3.16-3.19 (m, 2H), 2.81-2.86 (m, 1H), 2.49-2.54 (m, |
| | | | | 1H), 2.07-2.15 (m, 1H). |
| 148 | 320.3 | L2 | 2.72 | (CDCl₃) δ ppm: 7.52 (d, 2H), 7.21 (d, 2H), 7.12 (d, 1H), 7.03- |
| | | | | 7.11 (m, 2H), 6.91 (t, 1H), 5.46 (t, 1H), 3.75-3.80 (m, 1H), 3.41- |
| | | | | 3.48 (m, 1H), 3.29 (t, 2H), 2.88-2.93 (m, 1H), 2.53-2.59 (m, 1H), |
| | | | | 2.29-2.32 (m, 1H), 2.19 (q, 2H), 1.13 (t, 3H). |
| 151 | 319.2 | L2 | 2.51 | (DMSO-d₆) δ ppm: 8.57 (d, 1H), 7.52-7.55 (m, 1H), 7.46-7.48 |
| | | | | (m, 1H), 7.16 (d, 1H), 7.07-7.12 (m, 2H), 6.95-6.99 (m, 1H), |
| | | | | 6.29 (dd, 1H), 6.06-6.10 (m, 1H), 5.68 (dd, 1H), 5.65 (t, 1H), |
| | | | | 3.81-3.84 (m, 1H), 3.39-3.46 (m, 3H), 2.90-2.95 (m, 1H), 2.56- |
| | | | | 2.63 (m, 1H), 2.34-2.41 (m, 1H). |
| 149 | 344.2 | L2 | 2.63 | (DMSO-d₆) δ ppm: 8.53 (d, 1H), 8.20 (t, 1H), 7.71-7.74 (m, 1H), |
| | | | | 7.62 (d, 1 H), 7.13 (d, 1H), 6.75-7.03 (m, 4H), 6.17-6.24 (m, |
| | | | | 1H), 6.05 (dd, 1H), 5.57 (dd, 1H), 3.78-3.82 (m, 1H), 3.28-3.24 |
| | | | | (m, 1H), 3.19 (t, 2H), 2.84-2.90 (m, 1H), 2.53-2.57 (m, 1H), |
| | | | | 2.16-2.18 (m, 1H). |
| 152 | 346.3 | L2 | 2.64 | (CDCl₃) δ ppm: 8.53 (d, 1H), 7.60-7.63 (m, 1H), 7.52-7.56 (m, |
| | | | | 1H), 7.11 (d, 1H), 6.99-7.04 (m, 1H), 6.92 (d, 1H), 6.84-6.88 |
| | | | | (m, 1H), 6.61 (t, 1H), 5.49 (t, 1H), 3.73-3.77 (m, 1H), 3.26-3.40 |
| | | | | (m, 3H), 2.91-2.96 (m, 1H), 2.58-2.64 (m, 1H), 2.31-2.36 (m, |
| | | | | 1H), 2.19 (q, 2H), 1.37 (t, 3 H). |
| 159 | 321.3 | L2 | 2.57 | (CDCl₃) δ ppm: 9.85 (s, 1H), 7.78 (d, 2H), 7.28 (d, 2H), 7.15 (t, |
| | | | | 2H), 7.06 (t, 1H), 6.92 (t, 1H), 6.26 (dd, 1H), 6.00-6.06 (m, 1H), |
| | | | | 5.65 (dd, 1H), 5.56 (t, 1H), 3.82-3.86 (m, 1H), 3.31-3.47 (m, |
| | | | | 3H), 2.92-2.97 (m, 1H), 2.57-2.63 (m, 1H), 2.38-2.41 (m, 1H). |
| 118 | 299.2 | L2 | 2.83 | (DMSO-d₆) δ ppm: 8.28-8.15 (t, 1H), 6.93-6.97 (t, 1H), 6.86- |
| | | | | 6.87 (d, 1H), 6.62-6.65 (d, 1H), 6.52 (s, 1H), 6.43-6.46 (t, 1H), |
| | | | | 6.22-6.29 (m, 1H), 6.05-6.10 (dd, 1H), 5.57-5.60 (dd, 1H), 3.56 |
| | | | | (s, 1H), 3.23-3.26 (m, 1H), 3.09-3.14 (m, 2H), 2.78-2.81 (m, |
| | | | | 1H), 2.68-2.73 (m, 1H), 2.38-2.49 (m, 1H), 1.92-1.99 (m, 1H), |
| | | | | 1.76-1.78 (m, 2H), 1.61-1.69 (m, 3H), 1.34-1.47 (m, 4H), 1.12- |
| | | | | 1.19 (m, 1H). |
| 143 | 367.3 | L3 | 5.22 | (CDCl₃) δ ppm: 7.04-7.08 (m, 1H), 6.96 (d, 1H), 6.57-6.65 (m, |
| | | | | 2H), 6.28 (dd, 1H), 6.05-6.12 (m, 1H), 5.65-5.68 (d, 2H), 3.62- |
| | | | | 3.63 (m, 1H), 3.42-3.49 (m, 1H), 3.22-3.29 (m, 2H), 2.82-2.95 |
| | | | | (m, 2H), 2.50-2.56 (m, 1H), 2.20-2.23 (m, 1H), 1.90-2.09 (m, |
| | | | | 5H) 1.50-1.54 (m, 4H). |
| 144 | 367.3 | L3 | 4.97 | (CDCl₃) δ ppm: 7.05 (t, 1H), 6.96 (d, 1H), 6.63 (d, 1H), 6.58 (t, |
| | | | | 1H), 6.27 (dd, 1H), 6.05-6.12 (m, 1H), 5.64-5.67 (m, 2H), 3.66 |
| | | | | (t, 1H), 3.39-3.44 (m, 1H), 3.23-3.31 (m, 2H), 2.83-2.96 (m, |
| | | | | 2H), 2.14-2.23 (m, 1H), 2.49-2.55 (m, 1H), 2.31-2.34 (m, 1H), |
| | | | | 2.14-2.23 (m, 3H), 1.68-1.82 (m, 6H). |
| 145 | 335.3 | L2 | 2.79 | (DMSO-d₆) δ ppm: 8.54 (t, 1H), 6.97 (t, 1H), 6.90 (d, 1H), 6.76 |
| | | | | (d, 1H), 6.49 (t, 1H), 6.22-6.28 (m, 1H), 6.08 (dd, 1H), 5.58 (dd, |
| | | | | 1H), 3.85-3.89 (m, 1H), 3.11-3.32 (m, 2H), 2.69-2.79 (m, 2H), |
| | | | | 2.37-2.43 (m, 1H), 1.97-2.08 (m, 5H), 1.68-1.74 (m, 4H). |
| 119 | 359.2 | L2 | 3.13 | (DMSO-d₆) δ ppm: 8.86 (t, 1H), 7.62-7.64 (d, 2H), 7.32-7.34 (d, |
| | | | | 2H), 7.11-7.13 (d, 1H), 6.93-7.02 (m, 2H), 6.82-6.84 (m, 1H), |
| | | | | 4.14 (s, 1H), 3.77-3.80 (m, 1H), 3.23-3.28 (m, 1H), 3.12-3.15 |
| | | | | (m, 2H), 2.81-2.86 (m, 1H), 2.46-2.53 (m, 1H), 2.13-2.16 (m, |
| | | | | 1H). |
| 120 | 418.3 | L3 | 4.07 | (DMSO-d₆) δ ppm: 8.092-8.120 (t, 1H), 7.61-7.63 (d, 2H), 7.32- |
| | | | | 7.34 (d, 2H), 7.12-7.14 (d, 1H), 7.01-7.03 (m, 1H), 6.93-6.99 |
| | | | | (m, 1H), 6.81-6.85 (m, 1H), 6.52-6.59 (m, 1H), 6.01-6.04 (d, |
| | | | | 1H), 3.77-3.81 (m, 1H), 3.24-3.29 (m, 1H), 3.16-3.18 (m, 2H), |
| | | | | 2.96-2.98 (m, 2H), 2.82-2.87(m, 1H), 2.48-2.54 (m, 1H), 2.06- |
| | | | | 2.15 (m, 7H). |
| 121 | 385.2 | L2 | 3.04 | (DMSO-d₆) δ ppm: 7.62-7.64 (d, 2H), 7.33-7.35 (d, 2H), 7.12- |
| | | | | 7.15 (m, 2H), 6.94-7.01 (m, 2H), 6.81-6.85 (m, 1H), 3.84-3.88 |
| | | | | (m, 1H), 3.26-3.31 (m, 1H), 2.87-3.01 (m, 6H), 2.52-2.56 (m, |
| | | | | 1H), 2.11-2.14 (m, 1H). |
| 122 | 397.2 | L2 | 3.19 | (DMSO-d₆) δ ppm: 7.62-7.64 (d, 2H), 7.44 (m, 1H), 7.32-7.34 |
| | | | | (d, 2H), 7.11-7.13 (d, 1H), 6.93-7.03 (m, 2H), 6.81-6.85 (t, 1H), |
| | | | | 6.64-6.71 (m, 1H), 5.92-6.02 (m, 2H), 3.83-3.87 (dd, 1H), 3.24- |
| | | | | 3.30 (m, 1H), 2.84-2.89 (m, 3H), 2.55 (m, 1H), 2.11-2.13 (m, |
| | | | | 1H). |
| 123 | 397.3 | L1 | 2.77 | (CDCl₃) δ ppm: 7.51-755 (d, 2H), 7.26-7.30 (d, 2H), 7.09-7.11 |
| | | | | (d, 1H), 6.95-7.03 (m, 2H), 6.81-6.85 (t, 1H), 4.04-4.08 (t, 2H), |
| | | | | 3.79-3.83 (dd, 1H), 3.51-3.56 (t, 1H), 3.00-3.14 (m, 5H), 2.62- |
| | | | | 2.68 (m, 1H), 2.36-2.38 (m, 1H). |
| 124 | 428.2 | L1 | 2.40 | (DMSO-d₆) δ ppm: 7.60-7.62 (d, 2H), 7.32-7.34 (d, 2H), 7.11- |
| | | | | 7.13 (d, 1H), 6.96-7.03(m, 2H), 6.81-6.85 (m, 2H), 3.83-3.84 |
| | | | | (dd, 1H), 3.28 (m, 1H), 3.09-3.10 (t, 2H), 2.78-2.89 (m, 3H), |
| | | | | 2.58 (m, 5H), 2.55 (m, 1H), 1.90-2.08 (m, 2H). |
| 125 | 374.2 | L2 | 3.04 | (CDCl₃) δ ppm: 7.55-7.57 (d, 2H), 7.27-7.29 (d, 2H), 7.10-7.12 |
| | | | | (m, 1H), 7.00-7.04 (m, 1H), 6.93-6.96 (m, 1H), 6.83-6.87 (m, |
| | | | | 1H), 6.13-6.21 (t, 1H), 3.746-3.745 (m, 1H), 3.38-3.44 (m, 5H), |
| | | | | 2.95-3.01 (m, 1H), 2.59-2.66 (m, 1H), 2.37-2.41 (m, 1H). |
| 126 | 360.2 | L2 | 2.37 | (CDCl₃) δ ppm: 7.52-7.54 (d, 2H), 7.26-7.28 (d, 2H), 7.10-7.12 |
| | | | | (m, 1H), 6.98-7.01 (m, 2H), 6.82-6.86 (m, 1H), 3.81-3.85 (m, |
| | | | | 1H), 3.39-3.44 (m, 1H), 2.72-2.95 (m, 3H), 2.64-2.70 (m, 1H), |
| | | | | 2.55-2.62 (m, 2H), 2.44-2.47 (t, 2H), 2.01 (br s, 1H), 1.54 (br s, |
| | | | | 1H). |
| 127 | 346.2 | L2 | 3.07 | (CDCl₃) δ ppm: 7.52-7.54 (d, 2H), 7.28 (d, 2H), 7.10-7.12 (dd, |
| | | | | 1H), 6.97-7.04 (m, 2H), 6.82-6.86 (m, 1H), 3.82-3.83 (m, 1H), |
| | | | | 3.54-3.61 (m, 2H), 3.39-3.49 (m, 1H), 2.95-3.01 (m, 1H), 2.82- |
| | | | | 2.86 (m, 1H), 2.68-2.73 (m, 1H), 2.57-2.64 (m, 1H), 2.18-2.23 |
| | | | | (m, 1H), 1.29 (bs, 1H). |
| 128 | 375.2 | L3 | 5.39 | (DMSO-d₆) δ ppm: 7.51-7.53 (d 1H), 7.23-7.26 (d, 2H), 6.90- |
| | | | | 6.94 (m, 1H), 6.84-6.86 (d, 1H), 6.74-6.76 (d, 1H), 6.21-6.26 |
| | | | | (dd, 1H), 5.99-6.05 (m, 1H), 5.62-5.65 (dd, 1H), 5.49-5.56 (t, |
| | | | | 1H), 3.71-3.75 (m 1H), 3.30-3.47 (m, 3H), 2.83-2.88 (m, 1H), |
| | | | | 2.36-2.46 (m, 2H), 2.25 (s, 3H). |
| 129 | 451.2 | L2 | 3.40 | (CDCl₃) δ ppm: 7.51-7.53 (d, 2H), 7.28-7.32 (m, 2H), 7.20-7.23 |
| | | | | (m, 3H), 7.14-7.16 (d, 1H), 6.97-7.01 (m, 1H), 6.92-6.97 (d, |
| | | | | 1H), 6.74-6.76 (d, 1H), 6.16-6.21 (dd, 1H), 5.84-5.91 (m, 1H), |
| | | | | 5.59-5.62 (dd, 1H), 5.12-5.21 (t, 1H), 3.98 (s, 2H), 3.69-3.73 |
| | | | | (m, 1H), 3.26-3.38 (m, 2H), 3.11-3.18 (m, 1H), 2.75-2.81 (m, |
| | | | | 1H), 2.38-2.44 (m, 1H), 2.25-2.31 (m, 1H). |
| 130 | 453.2 | L2 | 3.41 | (CDCl₃) δ ppm: 7.51-7.53 (d, 2H), 7.26-7.32 (m, 2H), 7.19-7.23 |
| | | | | (m, 3H), 7.14-7.16 (d, 2H), 6.97-7.01 (m, 1H), 6.90-6.92 (m, |
| | | | | 1H), 6.74-6.76 (m, 1H), 5.04-5.12 (t, 1H), 3.98 (s, 2H), 3.68- |
| | | | | 3.72 (m, 1H), 3.31-3.36 (m, 1H), 3.17-3.26 (m, 1H), 3.06-3.11 |
| | | | | (m, 1H), 2.74-2.79 (m, 1H), 2.35-2.41 (m, 1H), 2.21-2.23 (m, |
| | | | | 1H), 2.01-2.07 (q, 2H), 1.03-1.07 (t, 3H). |
| 153 | 376.4 | L2 | 2.77 | (CDCl₃) δ ppm: 7.51-7.53 (d, 2H), 7.25-7.27 (t, 2H), 6.82-6.86 |
| | | | | (t, 1H), 6.42-6.44 (d, 1H), 6.29-6.31 (d, 1H), 6.21-6.25 (dd, 1H), |
| | | | | 5.98-6.05 (m, 1H), 5.62-5.65 (dd, 1H), 5.54 (br s, 1H), 3.70- |
| | | | | 3.73 (dd, 1H), 3.62 (s, 2H), 3.42-3.47 (m, 2H), 3.31-3.38 (m, |
| | | | | 1H), 2.66-2.72 (dd, 1H), 2.41-2.44 (m, 1H), 2.26-2.32 (dd, 1H). |
| 153-En1 | 376.4 | S4 | 5.77 | (CDCl₃) δ ppm: 7.52 (d, 2H), 7.27 (d, 2H), 6.84 (t, 1H), 6.43 (d, |
| | | | | 1H), 6.29 (d, 1H), 6.23 (dd, 1H), 5.98-6.05 (m, 1H), 5.64 (dd, |
| | | | | 1H), 5.52 (t, 1H), 3.70-3.73 (m, 1H), 3.62 (s, 2H), 3.41-3.47 (m, |
| | | | | 2H), 3.31-3.38 (m, 1H), 2.66-2.72 (m, 1H), 2.41-2.44 (m, 1H), |
| | | | | 2.26-2.32 (m, 1H). |
| 153-En2 | 376.4 | S4 | 7.06 | (CDCl₃) δ ppm: 7.52 (d, 2H), 7.26 (d, 2H), 6.84 (t, 1H), 6.43 (d, |
| | | | | 1H), 6.31 (d, 1H), 6.23 (dd, 1H), 5.98-6.05 (m, 1H), 5.64 (dd, |
| | | | | 1H), 5.53 (t, 1H), 3.70-3.73 (m, 1H), 3.62 (s, 2H), 3.41-3.47 (m, |
| | | | | 2H), 3.31-3.38 (m, 1H), 2.66-2.72 (m, 1H), 2.41-2.44 (m, 1H), |
| | | | | 2.27-2.33 (m, 1H). |
| 150 | 364.3 | L2 | 2.53 | (CDCl₃) δ ppm: 7.52 (d, 2H), 7.265 (d, 2H), 6.84 (t, 1H), 6.42 |
| | | | | (d, 1H), 6.29 (d, 1H), 5.46 (t, 1H), 3.62-3.72 (m, 3H), 3.23-3.42 |
| | | | | (m, 3H), 2.65-2.70 (m, 1H), 2.34-2.38 (m, 1H), 2.23-2.33 (m, |
| | | | | 1H), 1.95 (s, 3H). |
| 154 | 374.3 | L2 | 3.05 | (CDCl₃) δ ppm: 7.60 (d, 2H), 77.27 (d, 2H), 7.10-7.13 (m, 1H), |
| | | | | 7.03-7.05 (m, 2H), 5.59 (t, 1H), 3.73-3.77 (m, 1H), 3.37-3.73 |
| | | | | (m, 2H), 3.14-3.27 (m, 2H), 2.71-2.77 (m, 1H), 2.37-2.40 (m, |
| | | | | 1H), 2.00 (s, 3H). |
| 161 | 419.3 | L2 | 2.78 | (DMSO-d₆) δ ppm: 12.5 (br s, 1H), 8.30 (t, 1H), 7.60 (d, 2H), |
| | | | | 7.29 (d, 2H), 6.95 (t, 1H), 6.86 (d, 1H), 6.78 (d, 1H), 6.17-6.24 |
| | | | | (m, 1H), 6.05 (dd, 1H), 5.56 (dd, 1H), 3.76-3.80 (m, 1H), 3.54 |
| | | | | (d, 2H), 3.22-3.27 (m, 1H), 3.14-3.17 (m, 1H), 2.78-2.86 (m, |
| | | | | 1H), 2.38-2.44 (m, 1H), 2.09-2.19 (m, 1H). |
| 161-En1 | 419.3 | S5 | 1.70 | (DMSO-d₆) δ ppm: 8.49 (t, 1H), 7.59 (d, 2H), 7.29 (d, 2H), 6.94 |
| | | | | (t, 1H), 6.85 (d, 1H), 6.77 (d, 1H), 6.19-6.26 (m, 1H), 6.06 (dd, |
| | | | | 1H), 5.55 (dd, 1H), 3.74-3.78 (m, 1H), 3.47 (d, 2H), 3.16-3.23 |
| | | | | (m, 2H), 3.06-3.09 (m, 1 H), 2.71-2.82 (m, 1H), 2.43-2.49 (m, |
| | | | | 1H), 2.10-2.19 (m, 1H). |
| 161-En2 | 419.3 | S5 | 2.25 | (DMSO-d₆) δ ppm: 8.41 (t, 1H), 7.59 (d, 2H), 7.29 (d, 2H), 6.94 |
| | | | | (t, 1H), 6.85 (d, 1H), 6.77 (d, 1H), 6.18-6.25 (m, 1H), 6.06 (dd, |
| | | | | 1H), 5.55 (dd, 1H), 3.74-3.78 (m, 1H), 3.48 (d, 2H), 3.15-3.22 |
| | | | | (m, 2H), 3.03-3.06 (m, 1 H), 2.75-2.80 (m, 1H), 2.45-2.49 (m, |
| | | | | 1H), 2.12-2.21 (m, 1H). |
| 163 | 400.3 | L2 | 2.93 | (DMSO-d₆) δ ppm: 8.21 (t, 1H), 7.64 (d, 2H), 7.32 (d, 2H), 7.04 |
| | | | | (t, 1H), 6.92 (t, 2H), 6.16-6.23 (m, 1H), 6.06 (dd, 1H), 5.57 (dd, |
| | | | | 1H), 4.01 (s, 2H), 3.77-3.81 (m, 1H), 3.13-3.29 (m, 3H), 2.85 |
| | | | | (dd, 1H), 2.40-2.46 (m, 1H), 2.12-2.19 (m, 1H). |
| 168 | 459.3 | L2 | 2.76 | (DMSO-d₆) δ ppm: 12.3 (s, 1H) 8.21 (t, 1H), 7.63 (d, 2H), 7.31 |
| | | | | (d, 2H), 7.00 (t, 1H), 6.899 (d, 1H), 6.789 (d, 1H), 6.16-6.23 (m, |
| | | | | 1H), 6.06 (dd, 1H), 5.56 (dd, 1H), 3.86 (s, 2H), 3.76-3.80 (m, |
| | | | | 1H), 3.11-3.29 (m, 3H), 2.86-2.91 (m, 1H), 2.49-2.50 (m, 1H), |
| | | | | 2.10-2.19 (m, 1H). |
| 168-En1 | 459.3 | S6 | 1.57 | (DMSO-d₆) δ ppm: 12.3 (s, 1H) 8.29 (t, 1H), 7.62 (d, 2H), 7.30 |
| | | | | (d, 2H), 6.99 (t, 1H), 6.89 (d, 1H), 6.78 (d, 1H), 6.19-6.25 (m, |
| | | | | 1H), 6.06 (dd, 1H), 5.56 (dd, 1H), 3.76-3.81 (m, 3H), 3.24-3.27 |
| | | | | (m, 1H), 3.16-3.19 (m, 2H), 2.86-2.91 (m, 1H), 2.44-2.50 (m, |
| | | | | 1H), 2.14-2.16 (m, 1H). |
| 168-En2 | 459.3 | S6 | 2.88 | (DMSO-d₆) δ ppm: 12.3 (s, 1H) 8.29 (t, 1H), 7.62 (d, 2H), 7.30 |
| | | | | (d, 2H), 6.99 (t, 1H), 6.87 (d, 1H), 6.78 (d, 1H), 6.20-6.27 (m, |
| | | | | 1H), 6.06 (dd, 1H), 5.56 (dd, 1H), 3.76-3.81 (m, 3H), 3.24-3.28 |
| | | | | (m, 1H), 3.16-3.19 (m, 2H), 2.86-2.91 (m, 1H), 2.44-2.50 (m, |
| | | | | 1H), 2.14-2.16 (m, 1H). |
| 167 | 445.3 | L3 | 4.67 | |
| 156 | 405.3 | L2 | 2.80 | (DMSO-d₆) δ ppm: 12.92 (br s, 1H), 8.38 (t, 1H), 7.62 (d, 2H), |
| | | | | 7.30 (d, 2H), 7.24-7.27 (m, 1H), 7.07-7.08 (m, 2H), 6.17-6.24 |
| | | | | (m, 1H), 6.05 (dd, 1H), 5.56 (dd, 1H), 3.75-3.79 (m, 1H), 3.14- |
| | | | | 3.31 (m, 4H), 2.67-2.74 (m, 1H), 2.09-2.20 (m, 1H). |
| 156-En1 | 405.3 | S6 | 1.82 | (DMSO-d₆) δ ppm: 8.75 (t, 1H), 7.60 (d, 2H), 7.27 (d, 2H), 7.15- |
| | | | | 7.17 (m, 1H), 7.02-7.05 (m, 2H), 6.19-6.26 (m, 1H), 6.06 (dd, |
| | | | | 1H), 5.55 (dd, 1H), 3.72-3.76 (m, 1H), 3.22-3.26 (m, 2H), 2.93- |
| | | | | 3.03 (m 2H), 2.78-2.84 (m, 1H), 2.17-2.26 (m, 1H). |
| 156-En2 | 405.3 | S6 | 2.69 | (DMSO-d₆) δ ppm: 8.8 (t, 1H), 7.58 (d, 2H), 7.25 (d, 2H), 7.06- |
| | | | | 7.15 (m, 1H), 6.96-7.02 (m, 2H), 6.19-6.26 (m, 1H), 6.06 (dd, |
| | | | | 1H), 5.55 (dd, 1H), 3.69-3.74 (m, 1H), 3.40-3.48 (m, 1H), 3.16- |
| | | | | 3.32 (m 2H), 2.80-2.89 (m, 3H), 2.21-2.30 (m, 1H). |
| 155 | 393.3 | L2 | 2.72 | (DMSO-d₆) δ ppm: 13.5 (br s, 1H) 8.13 (t, 1H), 7.62 (d, 2H), |
| | | | | 7.29 (d, 2H), 7.23 (t, 1H), 7.06 (d, 2H), 3.72-3.79 (m, 1H), 2.99- |
| | | | | 3.20 (m, 4H), 2.64-2.71 (m, 1H), 2.07-2.09 (m, 1H), 1.78 (s, |
| | | | | 3H). |
| 158 | 508.3 | L3 | 5.36 | DMSO-d6) δ ppm: 12.06 (s, 1H), 8.23 (t, 1H), 7.67 (d, 2H), |
| | | | | 7.34 (d, 2H), 7.05-7.12 (m, 2H), 6.95 (d, 1H), 6.16-6.22 (m, 1H), |
| | | | | 6.05 (dd, 1H), 5.56 (dd, 1H), 3.70-3.78 (m, 1H), 3.07-3.32 (m, |
| | | | | 4H), 2.91-2.96 (m, 1H), 2.54-2.61 (m, 1H), 2.14-2.17 (m, 1H), |
| | | | | 1.13-1.23 (m, 4H). |
| 158-En1 | 508.3 | S7 | 4.62 | (DMSO-d₆) δ ppm: 8.31 (t, 1H), 7.57 (d, 2H), 7.25 (d, 2H), 6.92- |
| | | | | 7.02 (m, 3H), 6.26-6.33 (m, 1H), 6.05 (dd, 1H), 5.54 (dd, 1H), |
| | | | | 3.75-3.79 (m, 1H), 3.16-3.32 (m, 2H), 2.79-2.95 (m, 4H), 2.22- |
| | | | | 2.24 (m, 1H), 0.81-0.84 (m, 2H), 0.69-0.72 (m, 2H). |
| 158-En2 | 508.3 | S7 | 5.22 | (DMSO-d₆) δ ppm: 8.31 (t, 1H), 7.57 (d, 2H), 7.25 (d, 2H), 6.92- |
| | | | | 7.02 (m, 3H), 6.26-6.33 (m, 1H), 6.05 (dd, 1H), 5.54 (dd, 1H), |
| | | | | 3.75-3.79 (m, 1H), 3.16-3.25 (m, 2H), 2.79-2.95 (m, 4H), 2.22- |
| | | | | 2.24 (m, 1H), 0.81-0.84 (m, 2H), 0.69-0.72 (m, 2H). |
| 157 | 482.2 | L2 | 3.16 | (DMSO-d₆) δ ppm: 12.20 (br s, 1H), 8.27 (t, 1H), 7.63 (d, 2H), |
| | | | | 7.30 (d, 2H), 6.98-7.07 (m, 3H), 6.20-6.27 (m, 1H), 6.05 (dd, |
| | | | | 1H), 5.56 (dd, 1H), 3.76-3.80 (m, 1H), 3.13-3.26 (m, 6H), 2.90- |
| | | | | 2.96 (m, 1H), 2.65-2.69 (m, 1H), 2.16-2.18 (m, 1H). |
| 157-En1 | 482.2 | S7 | 4.73 | (DMSO-d₆) δ ppm: 8.31 (t, 1H), 7.58 (d, 2H), 7.25 (d, 2H), 6.93- |
| | | | | 7.02 (m, 3H), 6.26-6.33 (m, 1H), 6.05 (dd, 1H), 5.55-5.56 (dd, |
| | | | | 1H), 3.74-3.79 (m, 1H), 3.17-3.26 (m, 2H), 2.87-2.98 (m, 5H), |
| | | | | 2.75-2.81 (m, 1H), 2.20-2.21 (m, 1H). |
| 131 | 391.3 | L2 | 2.93 | (CDCl₃) δ ppm: 7.47-7.49 (d, 2H), 7.16-7.18 (d, 2H), 7.98-8.00 |
| | | | | (d, 1H), 6.68-6.88 (d, 1H), 6.62-6.65 (dd, 1H), 6.22-6.27 (dd, |
| | | | | 1H), 5.99-6.05 (m, 1H), 5.63-5.66 (dd, 1H), 5.46-5.54 (t, 1H), |
| | | | | 3.77 (s, 3H), 3.74-3.75 (m, 1H), 3.28-3.43 (m, 3H), 2.89-2.94 |
| | | | | (m, 1H), 2.55-2.61 (m, 1H), 2.33-2.36 (m, 1H). |
| 132 | 361.2 | L2 | 3.10 | (DMSO-d₆) δ ppm: 7.60 (d, 2H), 7.32 (d, 2H), 7.21 (d, 1H), |
| | | | | 7.03-7.10 (m, 2H), 6.90-6.94 (m, 1H), 6.70-6.79 (m, 1H), 6.03- |
| | | | | 6.12 (m, 1H), 5.58-5.70 (m, 1H), 4.40-4.76 (m, 1H), 3.57-3.88 |
| | | | | (m, 2H), 2.79-3.19 (m, 5H). |
| 133 | 405.3 | L2 | 3.21 | (DMSO-d₆) δ ppm: 7.59-7.613 (d, 2H), 7.30-7.32 (d, 2H), 7.19- |
| | | | | 7.21 (d, 1H), 7.03-7.10 (m, 2H), 6.90-6.94 (m, 1H), 6.71-6.78 |
| | | | | (m, 1H), 6.09-6.13 (m, 1H), 5.62-5.68 (m, 1H), 4.35-4.36 (m, |
| | | | | 1H), 3.41-3.84 (m, 6H), 3.06-3.23 (m, 4H), 2.89-2.93 (m, 1H). |
| 134 | 375.3 | L2 | 3.17 | (DMSO-d₆)) δ ppm: 7.61-7.64 (m, 2H), 7.33-7.36 (m, 2H), |
| | | | | 7.10-7.12 (d, 1H), 6.92-7.03 (m, 2H), 6.69-6.83 (m, 2H), 6.07- |
| | | | | 6.12 (dd, 1H), 5.56-5.68 (dd, 1H), 3.71-3.77 (m, 1H), 3.39-3.47 |
| | | | | (m, 2H), 3.26-6.31 (m, 1H), 2.89-3.05 (m, 3H), 2.78-2.89 (m, |
| | | | | 1H), 2.52-2.49 (m, 1H), 2.35-2.32 (m, 1H). |
| 135 | 419.3 | L2 | 3.26 | (DMSO-d6) δ ppm: 7.59-7.61 (d, 2H), 7.31-3.34 (d, 2H), 7.08- |
| | | | | 7.10 (d, 1H), 6.96-7.00 (m, 1H), 6.86-6.88 (d, 1H), 6.78-6.81 |
| | | | | (m, 1H), 6.64-6.71 (m, 1H), 6.04-6.09 (dd, 1H), 5.56-5.59 (dd, |
| | | | | 1H), 3.70-3.75 (m, 1H), 3.40-3.54 (m, 6H), 3.18 (s, 3H), 2.81- |
| | | | | 2.86 (m, 1H), 2.51-2.57 (m, 1H), 2.39-2.42 (m, 1H). |
| 170 (Int-10) | 307.2 | L5 | 4.25 | (CDCl3) δ ppm: 7.51-7.53 (d, 2H), 7.26-7.29 (m, 2H), 7.10-7.12 |
| | | | | (d, 1H), 7.00-7.01 (m, 2H), 6.81-6.85 (m, 1H), 3.82-3.85 (m, |
| | | | | 1H), 3.33-3.38 (m, 1H), 2.92-2.97 (m, 1H), 2.71-2.80 (m, 2H), |
| | | | | 2.52-2.59 (m, 1H), 2.07-2.10 (m, 1H). |
| 171 (Int-3) | 293.2 | L5 | 4.31 | (DMSO-d6) δ ppm: 7.58-7.63 (m, 2H), 7.31-7.36 (m, 2H), 7.09- |
| | | | | 7.14 (m, 1H), 6.95-7.02 (m, 2H), 6.80-6.86 (m, 1H), 3.75-3.81 |
| | | | | (m, 1H), 3.09-3.21 (m, 2H), 2.90-2.97 (m, 1H), 2.48-2.54 (m, |
| | | | | 1H), 1.63-1.80 (m, 2H). |
| 172 | 307.2 | L2 | 2.32 | (DMSO-d6) δ ppm: 8.35-8.37 (d, 1H), 7.31-7.43 (m, 5H), 7.22- |
| | | | | 7.24 (d, 1H), 7.02-7.08 (m, 1H), 6.93-6.99 (m, 1H), 6.84-6.91 |
| | | | | (brs, 1H), 6.18-6.38 (m, 1H), 6.02-6.09 (dd, 1H), 5.56-5.59 (dd, |
| | | | | 1H), 4.20-4.25 (m, 1H), 3.76-3.85 (m, 2H), 3.17-3.23 (m, 1H), |
| | | | | 2.76-2.82 (m, 1H). |
| 173 | 313.3 | L2 | 2.57 | (DMSO-d6) δ ppm: 8.30-8.32 (d, 1H), 7.28-7.46 (br s, 1H), |
| | | | | 7.09-7.26 (m, 3H), 6.12-6.17 (m, 1H), 6.08-6.09 (dd, 1H), 6.57- |
| | | | | 6.60 (dd, 1H), 4.13-4.15 (m, 1H), 3.68-3.78 (m, 2H), 3.07-3.13 |
| | | | | (m, 1H), 2.65-2.73 (m, 1H), 1.59-1.67 (m, 5H), 1.28-1.47 (m, |
| | | | | 2H), 1.05-1.25 (m, 3H). |
| 174 | 293.2 | L2 | 2.77 | (DMSO-d6) δ ppm: 8.13-8.15 (d, 1H), 7.19-7.32 (m, 5H), 6.90- |
| | | | | 6.95 (m, 2H), 6.50-6.53 (m, 2H), 6.25-6.32 (m, 1H), 6.07-6.11 |
| | | | | (m, 1H), 5.57-5.60 (dd, 1H), 4.43-4.54 (m, 2H), 4.19-4.23 (m, |
| | | | | 1H), 3.43-3.46 (m, 1H), 3.19-3.24 (m, 1H), 2.95-3.0 (dd, 1H), |
| | | | | 2.69-2.76 (m, 1H). |
| 175 | 299.2 | L2 | 3.15 | (DMSO-d6) δ ppm: 8.05-8.07 (d, 1H), 6.85-6.99 (m, 1H), 6.88- |
| | | | | 6.90 (m, 1H), 6.45-6.54 (m, 2H), 6.23-6.30 (m, 1H), 6.07-6.12 |
| | | | | (dd, 1H), 5.56-5.59 (dd, 1H), 4.07-4.09 (m, 1H), 3.33-3.36 (m, |
| | | | | 1H), 3.11-3.16 (m, 1H), 3.02-3.03 (d, 2H), 2.88-2.93 (m, 1H), |
| | | | | 2.63-2.69 (m, 1H), 1.62-1.71 (m, 6H), 1.10-1.17 (m, 3H), 0.91- |
| | | | | 0.94 (m, 2H). |
| 176 | 307.2 | L2 | 2.87 | (DMSO-d6) δ ppm: 8.11-8.13 (t, 1H), 7.30-7.32 (m, 2H), 7.19- |
| | | | | 7.29 (m, 3H), 6.44-6.49 (m, 2H), 6.20-6.27 (m, 1H), 6.05-6.10 |
| | | | | (dd, 1H), 5.56-5.59 (dd, 1H), 4.42-4.53 (q, 2H), 3.34-3.38 (m, |
| | | | | 1H), 3.09-3.21 (m, 3H), 2.76-2.81 (m, 1H), 2.47-2.53 (m, 1H), |
| | | | | 2.14-2.16 (m, 1H). |
| 177 | 321.2 | L2 | 2.40 | (DMSO-d6) δ ppm: 8.22-8.25 (t, 1H), 7.32-7.41 (m, 5H), 7.19- |
| | | | | 7.20 (m, 1H), 6.99-7.01 (m, 1H), 6.87-6.90 (m, 1H), 6.76-6.77 |
| | | | | (m, 1H), 6.16-6.23 (m, 1H), 6.04-6.09 (dd, 1H), 5.57-5.60 (dd, |
| | | | | 1H), 3.95-4.00 (m, 1H), 3.31-3.38 (m, 1H), 3.17-3.20 (m, 2H), |
| | | | | 2.91-2.96 (m, 1H), 2.55-2.61 (m, 1H), 2.21-2.24 (m, 1H). |
| 178 | 361.2 | L2 | 3.12 | (DMSO-d6) δ ppm: 8.36-8.39 (t, 1H), 7.58-7.61 (d, 2H), 7.33- |
| | | | | 7.35 (d, 2H), 7.14-7.16 (d, 1H), 7.00-7.04 (m, 1H), 6.93-6.95(d, |
| | | | | 1H), 6.84-6.88 (t, 1H), 6.20-6.26 (m, 1H), 6.09-6.13(dd, 1H), |
| | | | | 5.61-5.63 (dd, 1H), 4.07-4.10 (m, 1H), 3.36 (m, 1H), 3.18-3.24 |
| | | | | (m, 1H), 2.80-2.88 (m, . 1H), 2.67-2.71 (m, 1H), 1.83-1.93 (m, |
| | | | | 2H). |
| 179 | 313.3 | L2 | 3.23 | (CDCl3) δ ppm: 7.01-7.06 (m, 1H), 6.93-6.94 (m, 1H), 6.51- |
| | | | | 6.56 (m, 2H), 6.26-6.30 (dd, 1H), 6.06-6.12 (m, 1H), 5.64-5.67 |
| | | | | (dd, 2H), 3.41-3.48(m, 1H), 3.26-3.33 (m, 2H), 3.10-3.13 (m, |
| | | | | 1H), 3.01-3.08 (m, 2H), 2.81-2.85 (dd, 1H), 2.52-2.58 (m, 1H), |
| | | | | 2.25-2.26 (m, 1H), 1.68-1.76 (m, 6H), 1.16-1.19 (m, 3H), 0.92- |
| | | | | 0.95 (m, 2H). |
| 180 | 327.3 | L2 | 2.66 | (DMSO-d6) d ppm: 8.10-8.26 (t, 1H), 7.32 (br s, 1H), 7.08-7.21 |
| | | | | (m, 3H), 6.20-6.27 (m, 1H), 6.06-6.11 (dd, 1H), 5.59-5.62 (dd, |
| | | | | 1H), 3.90-3.95 (m, 1H), 3.12-3.22 (m, 3H), 2.72-2.85 (m, 2H), |
| | | | | 2.39-2.50 (m, 1H), 2.07-2.10 (m, 1H), 1.58-1.67 (m, 5H), 1.34- |
| | | | | 1.41 (m, 2H), 1.13-1.14 (m, 3H). |
| 181 | 361.2 | L2 | 3.01 | (CDCl3) δ ppm: 7.53-7.55 (d, 2H), 7.33-7.35 (d, 2H), 7.02-7.07 |
| | | | | (m, 2H), 6.67-6.71 (m, 1H), 6.53-6.55 (d, 1H), 6.25-6.30 (dd, |
| | | | | 1H), 5.99-6.06 (q, 1H), 5.75-5.77 (d, 1H), 5.63-5.66 (dd, 1H), |
| | | | | 4.48-4.62 (m, 3H), 3.58-3.61 (dd, 1H), 3.34-3.39 (m, 1H), 3.17- |
| | | | | 3.23 (m, 1H), 2.79-2.84 (m, 1H). |
| 182 | 361.2 | L2 | 3.00 | (DMSO-d6) δ ppm: 8.17-8.19 (d, 1H), 7.52-7.60 (m, 4H), 6.91- |
| | | | | 6.97 (m, 2H), 6.49-6.57 (m, 2H), 6.26-6.33 (m, 1H), 6.06-6.11 |
| | | | | (dd, 1H), 5.56-5.59 (dd, 1H), 4.64-4.52 (q, 2H), 4.21-4.25 (m, |
| | | | | 1H), 3.44-3.48 (m, 1H), 3.22-3.27 (m, 1H), 2.98-3.03 (m 1H), |
| | | | | 2.71-2.77 (m, 1H). |
| 183 | 415.2 | L1 | 2.49 | (DMSO-d6) δ ppm: 8.39 (br s, 1H), 7.65-7.67 (d, 2H), 7.34-7.37 |
| | | | | (d, 2H), 7.12-7.14 (d, 1H), 6.96-7.03 (m, 2H), 6.85-6.87 (t, 1H), |
| | | | | 3.92-3.96 (dd, 1H), 3.33 (m, 1H), 3.17-3.23 (t, 2H), 2.92-3.03 |
| | | | | (m, 3H), 2.79-2.82 (t, 2H), 2.59-2.63 (m, 1H), 2.31-2.33 (m, |
| | | | | 1H). |
| 184 | 375.2 | L2 | 2.98 | (CDCl3) δ ppm: 7.56-7.58 (d, 2H), 7.35-7.37 (d, 2H), 6.98-7.02 |
| | | | | (m, 2H), 6.62-6.65 (m, 1H), 6.46-6.48 (d, 1H), 6.23-6.28 (dd, |
| | | | | 1H), 6.00-6.07 (m, 1H), 5.63-5.66 (dd, 1H), 5.56-5.61 (t, 1H), |
| | | | | 4.51-4.52 (m, 2H), 3.34-3.48 (m, 3H), 3.14-3.19 (m, 1H), 2.91- |
| | | | | 2.96 (m, 1H), 2.60-2.66 (m, 1H), 2.36-2.38 (m, 1H). |
| 185 | 375.3 | L2 | 3.02 | (CDCl3) δ ppm: 7.50-7.52 (m, 2H), 7.43-7.44 (m, 2H), 6.99- |
| | | | | 7.02 (m, 2H), 6.62-6.66 (m, 1H), 6.49-6.50 (d, 1H), 6.23-6.27 |
| | | | | (dd, 1H), 6.00-6.07 (m, 1H), 5.63-5.66 (dd, 1H), 5.56-5.62 (t, |
| | | | | 1H), 4.50-4.52 (m, 2H), 3.33-3.47 (m, 3H), 3.13-3.18 (m, 1H), |
| | | | | 2.91-2.96 (m, 1H), 2.60-2.67 (m, 1H), 2.35-2.38 (m, 1H). |
| 186 | 411.2 | L2 | 2.79 | (CDCl3) δ ppm: 8.02-8.04 (d, 1H), 7.92 (s, 1H), 7.82-7.84 (m, |
| | | | | 1H), 7.64-7.69 (m, 2H), 7.24-7.26 (m, 1H), 7.11-7.15 (m, 1H), |
| | | | | 7.06-7.08 (m, 1H), 6.27-6.31 (dd, 1H), 5.99-6.05 (m, 1H), 5.65- |
| | | | | 5.68 (dd, 1H), 5.58-5.63 (d, 1H), 4.27-4.32 (m, 1H), 4.04-4.08 |
| | | | | (m, 1H), 3.74-3.79 (m, 1H), 2.81-2.87 (m, 1H), 2.59-2.65 (m, |
| | | | | 1H). |
| 187 | 411.2 | L3 | 4.74 | (DMSO-d6) δ ppm: 7.90-7.92 (d, 2H), 7.71-7.75 (m, 3H), 7.21- |
| | | | | 7.25 (m, 1H), 7.06-7.14 (m, 1H), 6.27-6.32 (dd, 1H), 5.98-6.05 |
| | | | | (m, 1H), 5.66-5.69 (dd, 1H), 5.57-5.58 (d, 1H), 4.27-4.33 (m, |
| | | | | 1H), 4.11-4.26 (m, 1H), 2.84-2.90 (m, 1H), 2.61-2.67 (m, 1H). |
| 188 | 363.2 | L2 | 3.06 | (CDCl3) δ ppm: 7.47-7.49 (d, 2H), 7.20-7.23 (d, 2H), 7.13-7.15 |
| | | | | (dd, 1H), 7.00-7.07 (m, 2H), 6.89-6.93 (m, 1H), 5.68 (s, 1H), |
| | | | | 4.16-4.18 (m, 1H), 3.52-3.59 (m, 1H), 3.19-3.26 (m, 1H), 2.79- |
| | | | | 2.83 (m, 2H), 2.20-2.26 (m, 2H), 2.03-2.06 (m, 1H), 1.88-1.90 |
| | | | | (m, 1H), 1.14-1.18 (t, 1H). |
| 189 | 375.2 | L2 | 3.04 | (CDCl3) δ ppm: 7.54-7.56 (d, 2H), 7.35-7.37 (d, 2H), 6.97-7.04 |
| | | | | (m, 1H), 6.63-6.67 (m, 1H), 6.44-6.46 (d, 1H), 6.17-6.22 (dd, |
| | | | | 1H), 5.90-5.97 (m, 1H), 5.59-5.62 (dd, 2H), 4.66 (s, 2H), 3.59- |
| | | | | 3.63 (m, 1H), 3.46-3.52 (m, 1H), 3.35-3.42 (m, 1H), 2.75-2.92 |
| | | | | (m, 2H), 2.00-2.01 (m, 2H). |
| 190 | 375.2 | L2 | 5.24 | (CDCl3) δ ppm: 7.49-7.52 (m, 2H), 7.39-7.44 (m, 2H), 6.98- |
| | | | | 7.04 (m, 2H), 6.64-6.68 (m, 1H), 6.46-6.48 (d, 1H), 6.17-6.22 |
| | | | | (dd, 1H), 5.90-5.96 (m, 1H), 5.59-5.62 (dd, 2H), 4.64 (s, 2H), |
| | | | | 3.58-3.63 (m, 1H), 3.45-3.51 (m, 1H), 3.36-3.43 (m, 1H), 2.86- |
| | | | | 2.94 (m, 1H), 2.76-2.82 (m, 1H), 2.00-2.02 (m, 2H). |
| 191 | 371.1 | L2 | 2.90 | (CDCl3) δ ppm: 7.39-7.42 (d, 2H), 7.09-7.11 (d, 2H), 6.99-7.07 |
| | | | | (m, 2H), 6.65-6.69 (m, 1H), 6.56-6.58 (d, 1H), 6.24-6.29 (dd, |
| | | | | 1H), 5.98-6.04 (m, 1H), 5.75-5.77 (d, 1H), 5.62-5.65 (dd, 1H), |
| | | | | 4.57-4. 61 (m, 1H), 4.38-4.47 (m, 2H), 3.54-3.57 (m, 1H), 3.30- |
| | | | | 3.35 (m, 1H), 3.15-3.20 (m, 1H), 2.77-2.82 (m, 1H). |
| 192 | 375.2 | L3 | 6.13; | (DMSO-d6) δ ppm: 8.05 (d, 1H), 7.60-7.62 (m, 2H), 7.28-7.34 |
| | | | 6.14 | (m, 2H), 7.12 (d, 1H), 6.93-7.01 (m, 2H), 6.81-6.85 (m, 1H), |
| | | | | 6.03-6.24 (m, 2H), 5.53-5.60 (m, 1H), 3.76-3.97 (m, 2H), 3.21- |
| | | | | 3.24 (m, 1H), 2.80-2.88 (m, 1H), 2.50-2.67 (m, 1H), 1.97-2.01 |
| | | | | (m, 1H), 1.09-1.14 (m, 3H); 1:1 mixture of diastereoisomers |
| 193 | 432.4 | L2 | 3.63 | (DMSO-d6) δ ppm: 8.15 (t, 1H), 8.10 (t, 1H), 7.64 (d, 2H), 7.34 |
| | | | | (d, 2H), 7.13 (d, 1H), 6.99-7.05 (m, 1H), 6.93-6.98 (m, 1H), 6.84 |
| | | | | (td, 1H), 6.54 (dt, 1H), 5.94 (dt, 1H), 3.75-3.84 (m, 3H), 3.27 |
| | | | | (dd, 1H), 3.18 (td, 2H), 2.84 (dd, 1H), 2.09-2.20 (m, 1H), 1.85 |
| | | | | (s, 3H), 1.25-1.30 (m, 1H). |
| 194 | 425.3 | L3 | 6.30 | (CDCl3) δ ppm: 7.53 (d, 2H), 7.27 (d, 2H), 7.14 (d, 1H), 7.08 |
| | | | | (d, 1H), 6.99-7.04 (m, 1H), 6.95 (d, 1H), 6.81-6.88 (m, 1H), |
| | | | | 4.56-4.74 (m, 1H), 3.71-3.95 (m, 1H), 3.45-3.56 (m, 1H), 3.16- |
| | | | | 3.37 (m, 1H), 2.88-3.12 (m, 2H), 2.56-2.87 (m, 1H), 2.32-2.41 |
| | | | | (m, 1H); mixture of diastereoisomers |
| 194(Dia1)- | 425.3 | S2 | 4.49 | (CDCl3) δ ppm: 7.53 (d, 2H), 7.25 (d, 2H), 7.14 (d, 1H), 6.99- |
| En1 | | | | 7.04 (m, 1H), 6.95 (d, 1H), 6.84-6.88 (m, 1H), 4.67-4.74 (m, |
| | | | | 1H), 3.72-3.77 (m, 1H), 3.44-3.49 (m, 1H), 3.17-3.24 (m, 1H), |
| | | | | 2.96-3.05 (m, 2H), 3.79-3.86 (m, 1H), 2.32-2.41 (m, 1H). |
| 194(Dia1)- | 425.3 | S2 | 5.94 | (CDCl3) δ ppm: 7.53 (d, 2H), 7.25 (d, 2H), 7.14 (d, 1H), 6.99- |
| En2 | | | | 7.04 (m, 1H), 6.95 (d, 1H), 6.84-6.88 (m, 1H), 4.67-4.74 (m, |
| | | | | 1H), 3.72-3.77 (m, 1H), 3.44-3.49 (m, 1H), 3.17-3.24 (m, 1H), |
| | | | | 2.97-3.06 (m, 2H), 2.78-2.86 (m, 1H), 2.32-2.41 (m, 1H). |
| 194(Dia2)- | 425.3 | S2 | 4.77 | (CDCl3) δ ppm: 7.55 (d, 2H), 7.28 (d, 2H), 7.08 (d, 1H), 6.99- |
| En1 | | | | 7.04 (m, 1H), 6.95 (d, 1H), 6.81-6.85 (m, 1H), 4.56-4.63 (m, |
| | | | | 1H), 3.89-3.93 (m, 1H), 3.52-3.57 (m, 1H), 3.28-3.35 (m, 1H), |
| | | | | 3.04-3.10 (m, 1H), 2.88-2.93 (m, 1H), 2.58-2.64 (m, 1H), 2.35- |
| | | | | 2.40 (m, 1H). |
| 194(Dia2)- | 425.3 | S2 | 5.26 | (CDCl3) δ ppm: 7.55 (d, 2H), 7.28 (d, 2H), 7.08 (d, 1H), 6.99- |
| En2 | | | | 7.02 (m, 1H), 6.95 (d, 1H), 6.81-6.85 (m, 1H), 4.56-4.63 (m, |
| | | | | 1H), 3.90-3.93 (m, 1H), 3.52-3.57 (m, 1H), 3.28-3.35 (m, 1H), |
| | | | | 3.04-3.10 (m, 1H), 2.88-2.94 (m, 1H), 2.58-2.64 (m, 1H), 2.35- |
| | | | | 2.39 (m, 1H). |

In all above cases the analytical LC/MS and ¹H NMR data collected for En2 matched the data for En1.

Part B: Experimental Biology Procedures

### Example 81: Activity of Compounds of the Invention in a Reporter Gene Assay for Measuring the Inhibition of YAP/TAZ-TEAD Transcription

Hek293T cells are cultured in DMEM supplemented with 10% fetal bovine serum, Sodium pyruvate, Sodium bicarbonate, L-glutamine. The cells are harvested and transiently transfected with TEAD-responsive element luciferase reporter. Transfected cells are plated in 384-wells plate containing pre-diluted compounds. After 24 hours incubation at 37° C./5% CO₂, assay plates were cooled down to RT and levelled to an equal volume per well, prior to the addition of 25 uL luciferase substrate SteadyLite (Perkin Elmer)/well. The plate was shaken for 10 min at 600 rpm, centrifuged for 1 min at 500 rpm and measured with an Envision reader (PerkinElmer). The amount of relative light units produced by the TEAD reporter is used to calculated percent of inhibition. The percent of reporter inhibition was calculated in the presence of a positive control inhibitor (100% inhibition) versus a condition with the presence of the vehicle basal activity of the reporter (0% inhibition). The ability of a test compound to inhibit this activity was determined as: Percentage inhibition=[1−((RLU determined in the presence of vehicle−RLU determined for sample with test compound present) divided by (RLU determined in the presence of vehicle−RLU determined for sample with positive control inhibitor))]*100

The activities of example compounds tested are depicted in the table below. The activity ranges A, B and C refer to IC50 values in the reporter gene assay as follows: “A”: IC₅₀<1 μM; “B”: 1 μM≤IC₅₀≤20 μM and “C”: IC₅₀>20 μM; NT=not tested.

TABLE 3
activities of compounds of the invention in the gene reporter
assay for measuring YAP/TAZ-TEAD transcription activity

| Cpd | IC₅₀ | Cpd | IC₅₀ | cpd | IC₅₀ | cpd | IC₅₀ |
|---|---|---|---|---|---|---|---|
| 001 | A | 058 | A | 099 | B | 133 | B |
| 001-En1 | A | 059 | A | 100 | C | 134 | A |
| 001-En2 | A | 060 | A | 101 | A | 135 | A |
| 002 | A | 061 | B | 102 | A | 170 (Int-10) | B |
| 003 | A | 062 | A | 104 | A | 171 (Int-3) | B |
| 004 | A | 063 | A | 105 | A | 172 | C |
| 005 | A | 064 | A | 106 | A | 173 | C |
| 006 | A | 065 | A | 107 | A | 174 | A |
| 007 | A | 066 | A | 108 | A | 175 | A |
| 008 | A | 067 | B | 109 | A | 176 | A |
| 009 | B | 068 | A | 110 | A | 177 | A |
| 010 | A | 069 | A | 111 | A | 178 | A |
| 011 | A | 070 | A | 112 | A | 179 | A |
| 012 | B | 071 | B | 113 | A | 180 | A |
| 013 | A | 072 | B | 114 | A | 181 | A |
| 014 | B | 073 | A | 115 | A | 182 | A |
| 015 | B | 074 | A | 116 | A | 183 | C |
| 016 | B | 075 | A | 117 | A | 184 | A |
| 017 | A | 075-En1 | A | 147 | A | 185 | A |
| 018 | A | 075-En2 | A | 148 | B | 186 | A |
| 019 | A | 076 | A | 151 | A | 187 | A |
| 020 | A | 077 | A | 149 | A | 188 | C |
| 021 | A | 078 | A | 152 | B | 189 | A |
| 022 | A | 079 | A | 159 | A | 190 | A |
| 023 | A | 136 | A | 118 | A | 191 | A |
| 024 | A | 137 | C | 143 | A | 192 | A |
| 025 | A | 138 | A | 144 | A | 193 | A |
| 026 | A | 139 | A | 145 | A | 194 | A |
| 027 | A | 166 | C | 119 | A | 194(Dia1)-En1 | A |
| 028 | A | 166-En1 | C | 120 | A | 194(Dia1)-En2 | C |
| 029 | A | 166-En2 | C | 121 | A | 194(Dia2)-En1 | B |
| 030 | A | 164 | A | 122 | A | 194(Dia2)-En2 | A |
| 031 | A | 162 | C | 123 | A | | |
| 142 | A | 162-En1 | C | 124 | B | | |
| 032 | A | 162-En2 | C | 125 | A | | |
| 033 | A | 160 | C | 126 | B | | |
| 034 | A | 160-En1 | C | 127 | A | | |
| 035 | A | 160-En2 | C | 128 | A | | |
| 036 | A | 169 | C | 129 | A | | |
| 037 | B | 165 | C | 130 | A | | |
| 038 | B | 080 | A | 153 | A | | |
| 039 | A | 081 | A | 153-En1 | A | | |
| 040 | A | 082 | B | 153-En2 | A | | |
| 041 | B | 083 | A | 150 | A | | |
| 042 | B | 140 | A | 154 | A | | |
| 043 | A | 146 | A | 161 | B | | |
| 043-En1 | B | 141 | A | 161-En1 | C | | |
| 043-En2 | A | 084 | A | 161-En2 | B | | |
| 044 | C | 084-En1 | A | 163 | A | | |
| 045 | B | 084-En2 | A | 168 | A | | |
| 045-En1 | B | 085 | A | 168-En1 | B | | |
| 045-En2 | B | 086 | A | 168-En2 | A | | |
| 046 | A | 087 | A | 167 | C | | |
| 047 | A | 088 | A | 156 | C | | |
| 048 | A | 089 | A | 156-En1 | C | | |
| 049 | B | 090 | A | 156-En2 | C | | |
| 050 | B | 091 | A | 155 | C | | |
| 051 | B | 092 | A | 158 | C | | |
| 052 | B | 093 | C | 158-En1 | C | | |
| 053 | B | 094 | A | 158-En2 | C | | |
| 054 | B | 095 | A | 157 | C | | |
| 055 | C | 096 | A | 157-En1 | C | | |
| 056 | NT | 097 | A | 131 | A | | |
| 057 | NT | 098 | A | 132 | A | | |

### Example 82: Activity of Compounds of the Invention in Mesothelioma Cell Line Proliferation Assays

Mesothelioma cell lines, NCI-1H226 and NCI-1H2052 (all sourced from the ATCC cell culture collection) are plated in 96-well plates (Corning® 96 Well White Polystyrene Microplate clear flat bottom, white polystyrene (TC-Treated)), at 1500 cells/well in full medium (RPMI 1640 ATCC modification with L-glutamine, HEPES, Phenol Red, Sodium Pyruvate, High glucose, Low sodium bicarbonate and 10% fetal bovine serum). Cells are incubated overnight at 37° C. in an incubator with 5% C₀₂. Then compounds, dissolved in DMSO, are added in dose-response. Cells are incubated with compound dilutions for another 6 days at 37° C. in an incubator with 5% C₀₂. Cell viability is quantitated using the ATPlite kit (Perkin-Elmer) and the luminescence is read-out using an Envision instrument (Perkin-Elmer). The amount of relative light units produced using the ATPlite kit is used to calculated percent of inhibition.

The activities of some example compounds are depicted in the table below. The activity ranges A, B and C refer to EC50 values in the mesothelioma cell line proliferation assay as described as follows: “A”: EC₅₀<1 μM; “B”: 1 μM s EC₅₀≤10 μM and “C”: EC₅₀≥10 μM; NT: not tested.

TABLE 4
activities of a selection of compounds in the
mesothelioma cell line proliferation assay

| Cpd no | H226 | H2025 |
|---|---|---|
| 001 | A | A |
| 021 | A | NT |
| 023 | A | NT |
| 028 | A | A |
| 029 | A | A |
| 030 | A | NT |
| 142 | A | A |
| 039 | A | A |
| 059 | A | NT |
| 060 | A | A |
| 064 | A | NT |
| 073 | A | NT |
| 075 | A | A |
| 075-En1 | A | A |
| 075-En2 | A | A |
| 077 | A | A |
| 078 | A | A |
| 139 | A | A |
| 164 | A | A |
| 080 | A | A |
| 081 | A | A |
| 083 | A | A |
| 140 | A | A |
| 084 | A | NT |
| 113 | A | NT |
| 147 | A | A |
| 149 | B | B |
| 143 | A | A |
| 128 | A | NT |
| 153 | A | A |
| 153-En1 | A | A |
| 153-En2 | A | NT |
| 150 | A | A |
| 154 | A | B |
| 163 | A | A |
| 134 | A | NT |
| 181 | A | NT |
| 184 | A | NT |
| 192 | A | A |
| 194 | A | C |
| 194(Dia1)-En1 | A | A |

### Example 83: Activity of Compounds of the Invention in Cancer Cell Line Proliferation Assays

All the cell lines are cultured in the media supplemented with 10-20% fetal bovine serum, in the temperature of 37° C., 5% CO₂ and 95% humidity. The indicated cell lines (all sourced from the ATCC cell culture collection) are plated in 96-Well Flat Clear Bottom Black Polystyrene TC-Treated Microplates with the final cell density of 4×10³ cells/well. Cells are incubated overnight and then compounds, dissolved in DMSO, are added in dose-response. Cells are incubated with compound dilutions for another 6 days. Cell viability is quantitated using CellTiter-Glo reagent (Perkin Elmer) and measured using an EnVision Multi Label Reader.

Table 5 shows the concentration at which growth is inhibited for 50% (G150) for a range of cancer cell lines derived from various solid tumor types, characterized by genetic alterations that result in activation of YAP/TAZ-TEAD activity. The compound Cpd. No. 001 showed strong inhibitory activity on the cell proliferation of the selected tumor cell lines.

TABLE 5
GI₅₀s for Cpd. No. 001 in a selection of cancer cell lines.

| Cell Line | | | GI₅₀ |
|---|---|---|---|
| Name | Tissue Origin | Relevant genotype | (μM) |
| KMRC3 | Kidney | LATS1 HOM DEL | 0.02 |
| NCI-H226 | Lung (mesothelioma) | NF2 HOM DEL/ | 0.04 |
| | | LATS1 HOM DEL | |
| SCC-9 | Head & neck (squamous) | FAT1 HOM DEL | 0.05 |
| RL95-2 | Endometrial (adeno- | LATS1 HOM DEL | 0.05 |
| | squamous) | | |
| C-33 A | Cervical (squamous) | L914Wfs*10 LATS2 | 0.1 |
| Caov-3 | Ovary | LATS2 HOM DEL | 0.11 |
| Calu-1 | Lung (squamous) | NF2 HOM DEL/ | 0.12 |
| | | LATS2 HOM DEL | |

### Example 84: In Vivo Efficacy of Cpd. No. 084 and Cpd. No. 001 in the Treatment of a Subcutaneous Human Lung Cancer Xenograft Model (NCI-H226-Mesothelioma) in Female BALB/c Nude Mice

The NCI-H226 tumor cells were maintained in vitro with RPMI-1640 medium supplemented with 10% fetal bovine serum at 37° C. in an atmosphere of 5% CO₂ in air. The cells in exponential growth phase were harvested and the cell numbers were quantitated by hemocytometer and cell viability was quantified by Trypan Blue counting before tumor inoculation. Balb/c nude mouse were inoculated with subcutaneously in the right front flank regions with 1×10⁷ of NCI-H226 tumor cells in 0.1 mL of PBS mixed with 0.1 ml of Matrigel (1:1) for tumor development. All animals were randomized based on the tumor sizes as well as body weights and randomly allocated to the different study groups (8 mice per group). The randomization started when the mean tumor size at the right rear flank region had reached approximately 172 mm³. The date of randomization was denoted as Day 0.

Cpd. No. 084 was formulated in Vehicle 1 (PEG400 (40%)+PG (30%)+WFI (30%)) as a 10 mg/ml solution, and dosed orally, twice daily, using 10 μl/g (final dose is 100 mg/kg) for 21 days. Cpd. No. 001 was formulated in Vehicle 2 (PEG400 (65%)+PG (20%)+WFI (15%)) as a 10 mg/ml solution, and dosed orally, twice daily, using 10 μl/g (final dose is 100 mg/kg) for 21 days. After tumor cell inoculation, the animals were checked daily for morbidity and mortality. At the time of routine monitoring, the animals were checked for any effects of tumor growth and treatments on behavior such as mobility, food and water consumption, body weight gain/loss (body weight was measured at least two times per week), and any other abnormalities. Mortality and observed clinical signs were recorded for individual animals. Tumor volumes were measured at least two times weekly in two dimensions using a caliper, and the volume was expressed in mm³ using the formula: V=(L×W×W)/2, where V is tumor volume, L is tumor length (the longest tumor dimension) and W is tumor width (the longest tumor dimension perpendicular to L).

Tumor growth inhibition (TGI) was used as an indicator of antitumor activities, and was calculated from the relative tumor volumes of the control and treatment groups due to the variance of tumor volumes among groups at Day 0. TGI (%)=(1-T/C)×100%; T/C=MTVₜ/MTV_{c}, MTVₜ: mean tumor volume in the treatment groups at a specific measurement day, MTV_{c}: mean tumor volume in the control group at a specific measurement day. Moreover, the difference of TGIs between control and treatment groups was analyzed at a measurement day when all or most of the mice in the control groups were alive. In addition, body weight changes over time in each group were used as an index to evaluate the tolerability of test drugs, and were calculated from the average percentage of body weight changes of each individual mouse relative to their initial body weight at randomization using the following formula: group BW %=mean of ((BWₜ−BW₀)/BW₀×100%), BWₜ: body weight at Day t, BW₀: body weight at Day 0.

To compare relative tumor volumes of different groups at a pre-specified day, we first used Bartlett's test to check the assumption of homogeneity of variance across all groups. When the p-value of Bartlett's test is >0.05, we run one-way ANOVA to test overall equality of means across all groups. If the p-value of the one-way ANOVA is <0.05, we further perform post hoc testing by running Tukey's HSD (honest significant difference) tests for all pairwise comparisons, and Dunnett's tests for comparing each treatment group with the vehicle group. When the p-value of Bartlett's test is <0.05, we run Kruskal-Wallis test to test overall equality of medians among all groups. If the p-value of the Kruskal-Wallis test is <0.05, we further perform post hoc testing by running Conover's non-parametric test for all pairwise comparisons or for comparing each treatment group with the vehicle group, both with single-step p-value adjustment. All tests were two-sided unless otherwise specified, and p-values<0.05 were regarded as statistically significant.

TABLE 6
results of the in vivo efficacy study
of Cpd. No. 001 and Cpd. No. 084.

| | Mean Tumor | | | |
|---|---|---|---|---|
| Treatment | Volume | T/C | TGI | |
| Description | (mm³)^{a} on day 20 | (%) | (%) | P value |
| Vehicle 1, p.o., | 425.69 ± 17.11 (8) | — | — | — |
| BID × 21 days | | | | |
| Cpd. No. 084, | 165.82 ± 15.42 (8) | 38.95 | 61.05 | <0.001 |
| 100 mg/kg, p.o., | | | | |
| BID × 21 days | | | | |
| Vehicle 2, p.o., | 437.87 ± 29.31 (8) | — | — | — |
| BID × 21 days | | | | |
| Cpd. No. 001, | 217.26 ± 45.21 (8) | 49.62 | 50.38 | 0.00289 |
| 100 mg/kg, p.o., | | | | |
| BID × 21 days | | | | |

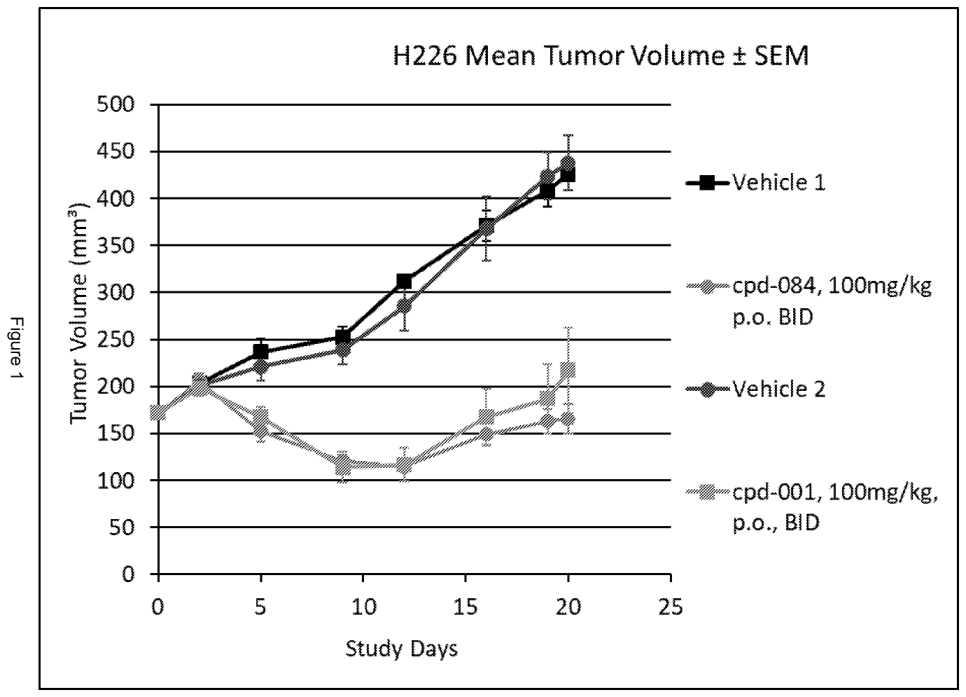

Both compounds Cpd. No. 001 and Cpd. No. 084 showed highly statistically significant and strong anti-tumor activity in the xenograft mesothelioma model as shown in table 6 and FIG. 1.

1. A compound of Formula (Ia), or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof, 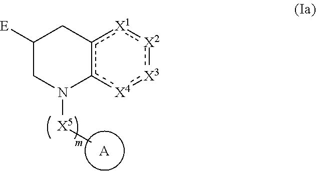
wherein:
E is selected from (5-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆ alkyl, —SO₂N(C₁₋₆alkyl)₂, —S(O)(NH)C₁₋₆alkyl, —S(O)(NC₁₋₆alkyl)C₁₋₆alkyl, —S(NH)(NH)C₁₋₆ alkyl, —NH₂, —NHC₁₋₆alkyl, and —N(C₁₋₆alkyl)₂; and —(CR^{10a}R^{10b})ₙ—NR¹R²;
n is selected from 0; 1; and 2;
m is selected from 0; and 1;
each — represents an optional double bond, whereby maximally 3 —are a double bond at the same time;
R¹ is selected from C₁₋₆alkyl; C₃₋₉cycloalkyl; C₂₋₆alkenyl; C₅₋₉cycloalkenyl; C₂₋₆alkynyl; C₅₋₉ cycloalkynyl; C₁₋₆heteroalkyl; C₂₋₆heteroalkenyl; C₂₋₆heteroalkynyl; —C(O)H; —C(O)R³; —C(O)OR⁴; —C(O)NR⁵R⁶; —S(O)₂R^{3a}; —S(O)R^{4a}; —S(O)₂NR^{5a}R^{6a}; —S(O)(NR^{5a})R^{4a}; —S(NR^{5a})(NR^{6a})R^{3a}; and —P(O)R^{5b}R^{6b};
wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆ alkyl)₂, —S(O)(NH)C₁₋₆alkyl, —S(O)(NC₁₋₆alkyl)C₁₋₆alkyl, —S(NH)(NH)C₁₋₆alkyl, —S(O)(NH)C₁₋₆alkyl, —S(O)(NC₁₋₆alkyl)C₁₋₆alkyl, —S(O)₂OH, —S(NH)(NH)C₁₋₆alkyl, —NH₂, —NHC₁₋₆alkyl, —N(C₁₋₆alkyl)₂;
R² is selected from hydrogen; C₁₋₆alkyl; C₃₋₉cycloalkyl; and C₁₋₆heteroalkyl;
R¹ and R² can be taken together to form a (4-; 5-; 6- or 7-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆ alkyl)₂, —S(O)(NH)C₁₋₆alkyl, —S(O)(NC₁₋₆alkyl)C₁₋₆alkyl, —S(NH)(NH)C₁₋₆alkyl, —NH₂, —NHC₁₋₆alkyl, —N(C₁₋₆alkyl)₂;
each R³ and R^{3a} is independently selected from hydroxyl; C₁₋₆alkyl; C₃₋₉cycloalkyl; C₂₋₆ alkenyl; C₅₋₉cycloalkenyl; C₂₋₆alkynyl; C₅₋₉cycloalkynyl; C₁₋₆heteroalkyl; C₂₋₆heteroalkenyl; and C₂₋₆heteroalkynyl;
wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁴ and R^{4a} is independently selected from C₁₋₆alkyl; C₃₋₉cycloalkyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; and C₂₋₆ heteroalkynyl;
wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁵, R^{5a}, R^{5b}, R⁵, R^{5a} and R^{5b} is independently selected from hydrogen; C₁₋₆alkyl; C₃₋₆ cycloalkyl; C₂₋₆alkenyl; C₅₋₉cycloalkenyl; C₂₋₆alkynyl; C₅₋₉cycloalkynyl; C₁₋₆heteroalkyl; C₂₋₆ heteroalkenyl; and C₂₋₆heteroalkynyl;
wherein said C₁₋₆alkyl, C₃₋₉cycloalkyl, C₂₋₆alkenyl, C₅₋₉cycloalkenyl, C₂₋₆alkynyl, C₅₋₉cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
and wherein each R⁵ and R⁶ or R^{5a} and R^{6a} can be taken together in order to form a (4-, 5-, 6-, or 7-membered) heterocycle which can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O— alkyl, —OCF₃, —CHF₂, —OCHF₂, cyano, nitro, —C(O)OH; NH₂; —NHalkyl, and —N(alkyl)₂;
cycle A is selected from aryl; heteroaryl; C₃₋₉cycloalkyl; and heterocycle;
wherein said aryl, heteroaryl, C₃₋₉cycloalkyl and heterocycle is substituted with one or more R⁷;
each R⁷ is independently selected from halogen; hydroxyl; sulfhydryl; ═O; ═S; —OZ¹; —SZ¹; —SCF₃; —SF₅; —CF₃; —OCF₃; —CHF₂; —OCHF₂; —NZ³Z⁴; —NZ³C(O)Z¹; cyano; —C(O)Z²; —C(O)OZ¹; —C(O)NZ³Z⁴; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylalkynyl; arylC₁₋₆ heteroalkyl; arylC₂₋₆heteroalkenyl; arylC₂₋₆heteroalkynyl; heteroarylC₁₋₆alkyl; heteroarylC₂₋₆ alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆ alkynyl; heterocycle-C₁₋₆heteroalkyl; heterocycle-C₂₋₆heteroalkenyl; and heterocycle-C₂₋₆ heteroalkynyl;
wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆ heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆ alkyl, —OCF₃, —CHF₂; —OCHF₂, cyano, nitro, —C(O)OH, —NH₂, —NHC₁₋₆alkyl, and —N(C₁₋₆ alkyl)₂;
X¹ is selected from CR⁸; N; and NR^{8a}; whereby X¹ can only be NR^{6a} when X² and/or X⁴ are C═O or C═S;
X² is selected from CR⁹; N; and NR^{5a}; whereby X² can only be NR^{9a} when X¹ and/or X³ are C═O or C—SH;
X³ is selected from CH; and N;
X⁴ is selected from CH; and N;
X⁵ is selected from —S(═O)₂—; —C(═O)—; and —CH₂—;
each R^{10a} is independently selected from hydrogen; and C₁₋₄alkyl;
each R^{10b} is independently selected from hydrogen; and C₁₋₄alkyl
wherein said alkyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
whereby maximally 2 of X¹, X², X³ and X⁴ can be a N (selected from N and NR^{8a} for X¹, from N and NR^{9a} for X², from N for X³, and from N for X⁴ respectively) at the same time;
provided that at least one of R⁸ and R⁹ is not hydrogen, each R⁸ and R⁹ are independently selected from hydrogen; halogen; hydroxyl; sulfhydryl; ═O; ═S; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{1a}; —NZ^{3a}C(O)Z^{1a}; —NZ^{3a}C(O)NZ^{3a}Z⁴a cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆ heteroalkenyl; arylC₂₋₆heteroalkynyl; heteroarylC₁₋₆alkyl; heteroarylC₂₋₆alkenyl; heteroarylC₂₋₆ alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆ heteroalkyl; heterocycle-C₂₋₆heteroalkenyl; and heterocycle-C₂₋₆heteroalkynyl;
wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each R^{8a} and R^{9a} are independently selected from hydrogen; hydroxyl; sulfhydryl; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{2a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each Z¹ and Z^{1a} is independently selected from C₁₋₆alkyl; C₃₋₉cycloakyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆heteroalkenyl; heteroarylC₂₋₆ heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆heteroalkyl, arylC₂₋₆ heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each Z² and Z^{2a} is independently selected from hydroxyl; C₁₋₆alkyl; C₃₋₉cycloakyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; C₂₋₆ heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆heteroalkyl, arylC₂₋₆ heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂;
each Z³, Z^{3a}, Z⁴, and Z^{4a} is independently selected from hydrogen; C₁₋₆alkyl; C₃₋₉cycloakyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆ alkenyl; arylC₂₋₆alkynyl; arylC₁₋₆heteroalkyl; arylC₂₋₆heteroalkenyl; arylC₂₋₆ heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
wherein said C₁₋₆alkyl, C₃₋₉cycloakyl, C₂₋₆alkenyl, C₅₋₉cycloalkenyl, C₂₋₆alkynyl, C₅₋₉cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆ heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆ alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆ alkyl, and —N(C₁₋₆alkyl)₂;
and wherein each Z³ and Z⁴ or Z^{3a} and Z^{4a}can be taken together in order to form a (4-, 5-, 6-, or 7-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂.

2. A compound of formula (I), or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof, 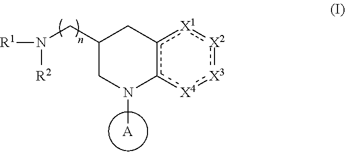
wherein:
n is selected from 0; 1; and 2;
each — represents an optional double bond, whereby maximally 3 —are a double bond at the same time;
R¹ is selected from C₁₋₆alkyl; C₃₋₉cycloalkyl; C₂₋₆alkenyl; C₅₋₉cycloalkenyl; C₂₋₆alkynyl; C₅₋₉ cycloalkynyl; C₁₋₆heteroalkyl; C₂₋₆heteroalkenyl; C₂₋₆heteroalkynyl; —C(O)H; —C(O)R³; —C(O)OR⁴; —C(O)NR⁵R⁶; —S(O)₂R^{3a}; —S(O)R^{4a}; —S(O)₂NR^{5a}R^{6a}; —S(O)(NR^{5a})R^{4a}; —S(NR^{5a})(NR^{6a})R^{3a}; and —P(O)R^{5b}R^{6b};
wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from C₁₋₆alkyl, C₃₋₉ cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆ alkyl)₂, —S(O)(NH)C₁₋₆alkyl, —S(O)(NC₁₋₆alkyl)C₁₋₆alkyl, —S(NH)(NH)C₁₋₆alkyl, —S(O)(NH)C₁₋₆alkyl, —S(O)(NC₁₋₆alkyl)C₁₋₆alkyl, —S(NH)(NH)C₁₋₆alkyl, —NH₂, —NHC₁₋₆ alkyl, —N(C₁₋₆alkyl)₂;
R² is selected from hydrogen; C₁₋₆alkyl; C₃₋₉cycloalkyl; and C₁₋₆heteroalkyl;
R¹ and R² can be taken together to form a (4-; 5-; 6- or 7-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, trifluoromethyl, —O—C₁₋₆alkyl, —OCF₃, cyano, nitro, —C(O)OH, —C(O)OC₁₋₆alkyl, —C(O)C₁₋₆alkyl, —CONH₂, —CONHC₁₋₆alkyl, —CON(C₁₋₆alkyl)₂, —SO₂C₁₋₆alkyl, —SO₂NH₂, —SO₂NHC₁₋₆alkyl, —SO₂N(C₁₋₆ alkyl)₂, —S(O)(NH)C₁₋₆alkyl, —S(O)(NC₁₋₆alkyl)C₁₋₆alkyl, —S(NH)(NH)C₁₋₆alkyl, —NH₂, —NHC₁₋₆alkyl, —N(C₁₋₆alkyl)₂;
each R³ and R^{3a} is independently selected from hydroxyl; C₁₋₆alkyl; C₃₋₉cycloalkyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; and C₂₋₆ heteroalkynyl;
wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁴ and R^{4a} is independently selected from C₁₋₆alkyl; C₃₋₉cycloalkyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; and C₂₋₆ heteroalkynyl;
wherein said C₁₋₆alkyl, C₃₋₉cycloalkyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
each R⁵, R⁵, R^{5b}, R⁶, R^{6a} and R^{6b} is independently selected from hydrogen; C₁₋₆alkyl; C₃₋₆ cycloalkyl; C₂₋₆alkenyl; C₅₋₉cycloalkenyl; C₂₋₆alkynyl; C₅₋₉cycloalkynyl; C₁₋₆heteroalkyl; C₂₋₆ heteroalkenyl; and C₂₋₆heteroalkynyl;
wherein said C₁₋₆alkyl, C₃₋₉cycloalkyl, C₂₋₆alkenyl, C₅₋₉cycloalkenyl, C₂₋₆alkynyl, C₅₋₉cycloalkynyl, C₁₋₆heteroalkyl, C₂₋₆heteroalkenyl and C₂₋₆heteroalkynyl can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂;
and wherein each R⁵ and R⁶ or R^{5a} and R^{6a} can be taken together in order to form a (4-, 5-, 6-, or 7-membered) heterocycle which can be unsubstituted or substituted with one or more substituents selected from alkyl, cycloalkyl, alkenyl, cycloalkenyl, alkynyl, cycloalkynyl, hydroxyl, ═O, halogen, —SH, ═S, —CF₃, —O— alkyl, —OCF₃, —CHF₂, —OCHF₂, cyano, nitro, —C(O)OH; NH₂; —NHalkyl, and —N(alkyl)₂;
cycle A is selected from aryl; heteroaryl; C₃₋₉cycloalkyl; and heterocycle;
wherein said aryl, heteroaryl, C₃₋₉cycloalkyl and heterocycle is substituted with one or more R⁷;
each R⁷ is independently selected from halogen; hydroxyl; sulfhydryl; ═O; ═S; —OZ¹; —SZ¹; —SCF₃; —SF₅; —CF₃; —OCF₃; —CHF₂; —OCHF₂; —NZ³Z⁴; —NZ³C(O)Z¹; cyano; —C(O)Z²; —C(O)OZ¹; —C(O)NZ³Z⁴; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylalkynyl; arylC₁₋₆ heteroalkyl; arylC₂₋₆heteroalkenyl; arylC₂₋₆heteroalkynyl; heteroarylC₁₋₆alkyl; heteroarylC₂₋₆ alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆ alkynyl; heterocycle-C₁₋₆heteroalkyl; heterocycle-C₂₋₆heteroalkenyl; and heterocycle-C₂₋₆ heteroalkynyl;
wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆ heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆ alkyl, —OCF₃, —CHF₂; —OCHF₂, cyano, nitro, —C(O)OH, —NH₂, —NHC₁₋₆alkyl, and —N(C₁₋₆ alkyl)₂;
X¹ is selected from CR⁸; N; and NR^{8a}; whereby X¹ can only be NR^{8a} when X² and/or X⁴ are C═O or C═S;
X² is selected from CR⁹; N; and NR^{9a}; whereby X² can only be NR^{9a} when X¹ and/or X³ are C═O or C—SH;
X³ is selected from CH; and N;
X⁴ is selected from CH; and N;
whereby maximally 2 of X¹, X², X³ and X⁴ can be a N (selected from N and NR^{8a} for X¹, from N and NR^{9a} for X², from N for X³, and from N for X⁴ respectively) at the same time;
provided that at least one of R⁸ and R⁹ is not hydrogen, each R⁸ and R⁹ are independently selected from hydrogen; halogen; hydroxyl; sulfhydryl; ═O; ═S; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{1a}; —NZ^{3a}C(O)Z^{1a}; —NZ^{3a}C(O)NZ^{3a}Z⁴a cyano; —C(O)Z^{2a}; —C(O)OZ^{1a}; —C(O)NZ^{3a}Z^{4a}; —C(O)H; —P(O)Z^{3a}Z^{4a}; C-alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆ heteroalkenyl; arylC₂₋₆heteroalkynyl; heteroarylC₁₋₆alkyl; heteroarylC₂₋₆alkenyl; heteroarylC₂₋₆ alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆ heteroalkyl; heterocycle-C₂₋₆heteroalkenyl; and heterocycle-C₂₋₆heteroalkynyl;
wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each R^{8a} and R^{9a} are independently selected from hydrogen; hydroxyl; sulfhydryl; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{2a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆alkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆ alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each Z¹ and Z^{1a} is independently selected from C₁₋₆alkyl; C₃₋₉cycloakyl; C₂₋₆alkenyl; C₅₋₉ cycloalkenyl; C₂₋₆alkynyl; C₅₋₉cycloalkynyl; C₁₋₆heteroalkyl; C₂₋₆heteroalkenyl; C₂₋₆ heteroalkynyl; aryl; heteroaryl; heterocycle; arylC₁₋₆alkyl; arylC₂₋₆alkenyl; arylC₂₋₆alkynyl; arylC₁₋₆ heteroalkyl; arylC₂₋₆heteroalkenyl; arylC₂₋₆heteroalkynyl; heteroarylC₁₋₆alkyl; heteroarylC₂₋₆ alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆ alkynyl; heterocycle-C₁₋₆heteroalkyl; heterocycle-C₂₋₆heteroalkenyl; and heterocycle-C₂₋₆ heteroalkynyl;
wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆heteroalkyl, arylC₂₋₆ heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂;
each Z² and Z^{2a} is independently selected from hydroxyl; C₁₋₆alkyl; C₃₋₉cycloakyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; C₂₋₆ heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
wherein said C₁₋₆alkyl, C₃₋₉cycloakyl, C₂₋₆alkenyl, C₅₋₉cycloalkenyl, C₂₋₆alkynyl, C₅₋₉ cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆heteroalkyl, arylC₂₋₆ heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆alkyl, and —N(C₁₋₆alkyl)₂;
each Z³, Z^{3a}, Z⁴, and Z^{4a} is independently selected from hydrogen; C₁₋₆alkyl; C₃₋₉cycloakyl; C₂₋₆alkenyl; C₅₋₉cycloalkenyl; C₂₋₆alkynyl; C₅₋₉cycloalkynyl; C₁₋₆heteroalkyl; C₂₋₆heteroalkenyl; C₂₋₆heteroalkynyl; aryl; heteroaryl; heterocycle; arylC₁₋₆alkyl; arylC₂₋₆ alkenyl; arylC₂₋₆alkynyl; arylC₁₋₆heteroalkyl; arylC₂₋₆heteroalkenyl; arylC₂₋₆ heteroalkynyl; heteroarylC₁₋₆alkyl; heteroarylC₂₋₆alkenyl; heteroarylC₂₋₆alkynyl; heteroarylC₁₋₆heteroalkyl; heteroarylC₂₋₆heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
wherein said C₁₋₆alkyl, C₃₋₉cycloakyl, C₂₋₆alkenyl, C₅₋₉cycloalkenyl, C₂₋₆alkynyl, C₅₋₉cycloalkynyl, C₁₋₆heteroalkyl, C₂₋₆heteroalkenyl, C₂₋₆heteroalkynyl, aryl, heteroaryl, heterocycle, arylC₁₋₆alkyl, arylC₂₋₆alkenyl, arylC₂₋₆alkynyl, arylC₁₋₆ heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆heteroalkyl, heteroarylC₂₋₆heteroalkenyl, heteroarylC₂₋₆heteroalkynyl, heterocycle-C₁₋₆alkyl, heterocycle-C₂₋₆alkenyl, heterocycle-C₂₋₆alkynyl, heterocycle-C₁₋₆heteroalkyl, heterocycle-C₂₋₆heteroalkenyl, and heterocycle-C₂₋₆heteroalkynyl can be unsubstituted or substituted with one or more substituents selected from C₁₋₆ alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH; NH₂; —NHC₁₋₆ alkyl, and —N(C₁₋₆alkyl)₂;
and wherein each Z³ and Z⁴ or Z^{3a} and Z^{4a} can be taken together in order to form a (4-, 5-, 6-, or 7-membered) heterocycle which can be unsubstituted or substituted with one or more substituents selected from C₁₋₆alkyl, C₃₋₉cycloalkyl, C₂₋₆alkenyl, C₂₋₆alkynyl, hydroxyl, ═O, halogen, —SH, ═S, —CF₃, —O—C₁₋₆alkyl, —OCF₃, —CHF₂, —OCHF₂, cyano, nitro, —C(O)OH;
NH₂; —NHC₁₋₆alkyl, and —N(C₁₋₆alkyl)₂.

3. The compound according to claim 1 or 2, wherein n is 0.

4. The compound according to claim 1 or 2, wherein n is 1.

5. The compound according to claims 1 to 4, wherein R¹ is selected from —C(O)R³; and —S(O)₂R^{3a}.

6. The compound according to claims 1 to 5, wherein R³ and R^{3a} are independently selected from C₂₋₆alkenyl; C₅₋₉cycloalkenyl; C₂₋₆alkynyl; and C₅₋₉cycloalkynyl;
wherein said C₂₋₆alkenyl, C₅₋₉cycloalkenyl, C₂₋₆alkynyl, and C₅₋₉cycloalkynyl can be unsubstituted or substituted with one or more substituents selected from alkyl, cycloalkyl, alkenyl, alkynyl, hydroxyl, ═O, halogen, —SH, ═S, —CF₃, —O-alkyl, —OCF₃, —CHF₂, —OCHF₂, cyano, nitro, —C(O)OH; NH₂; —NHalkyl, and —N(alkyl)₂.

7. The compound according to claims 1 to 6, wherein cycle A is heteroaryl.

8. The compound according to claims 1 to 6, wherein cycle A is aryl.

9. The compound according to claims 1 to 7, wherein R⁸ is selected from hydroxyl; sulfhydryl; —OZ^{1a}; —SZ^{1a}; —SCF₃; —SF₅; —S(O)Z^{1a}; —S(O)(NZ^{3a})Z^{1a}; —S(NZ^{3a})(NZ^{3a})Z^{1a}; —S(O)₂Z^{2a}; —S(O)₂NZ^{3a}Z^{4a}; —CF₃; —OCF₃; —CHF₂; —OCHF₂; nitro; —NZ^{3a}Z^{4a}; —NZ^{3a}S(O)₂Z^{2a}; —NZ^{3a}C(O)Z^{1a}; —NZ^{3a}C(O)NZ^{3a}Z^{4a}; cyano; —C(O)Z^{2a}; —C(O)OZ^{1a}; —C(O)NZ^{3a}Z^{4a}; —C(O)H; —P(O)Z^{3a}Z^{4a}; C₁₋₆alkyl; C₃₋₉cycloakyl; C₂₋₆alkenyl; C₂₋₆alkynyl; C₁₋₆heteroalkyl; C₂₋₆ heteroalkenyl; C₂₋₆heteroalkynyl; aryl; heteroaryl; heterocycle; arylC₁₋₆alkyl; arylC₂₋₆alkenyl; arylC₂₋₆alkynyl; arylC₁₋₆heteroalkyl; arylC₂₋₆heteroalkenyl; arylC₂₋₆heteroalkynyl; heteroarylC₁₋₆ alkyl; heteroarylC₂₋₆alkenyl; heteroarylC₂₋₆alkynyl; heteroarylC₁₋₆heteroalkyl; heteroarylC₂₋₆ heteroalkenyl; heteroarylC₂₋₆heteroalkynyl; heterocycle-C₁₋₆alkyl; heterocycle-C₂₋₆alkenyl; heterocycle-C₂₋₆alkynyl; heterocycle-C₁₋₆heteroalkyl; heterocycle-C₂₋₆heteroalkenyl; and heterocycle-C₂₋₆heteroalkynyl;
wherein said C₁₋₆alkyl, C₃₋₉cycloakyl, C₂₋₆alkenyl, C₂₋₆alkynyl, C₁₋₆heteroalkyl, C₂₋₆ heteroalkenyl, C₂₋₆heteroalkynyl, aryl, heteroaryl, heterocycle, arylC₁₋₆alkyl, arylC₂₋₆alkenyl, arylalkynyl, arylC₁₋₆heteroalkyl, arylC₂₋₆heteroalkenyl, arylC₂₋₆heteroalkynyl, heteroarylC₁₋₆alkyl, heteroarylC₂₋₆alkenyl, heteroarylC₂₋₆alkynyl, heteroarylC₁₋₆ heteroalkyl, heteroarylC₂₋₆heteroalkenyl, heteroarylC₂₋₆heteroalkynyl, heterocycle-C₁₋₆ alkyl, heterocycle-C₂₋₆alkenyl, heterocycle-C₂₋₆alkynyl, heterocycle-C₁₋₆heteroalkyl, heterocycle-C₂₋₆heteroalkenyl and heterocycle-C₂₋₆heteroalkynyl can be unsubstituted or substituted with one or more substituents selected from C₁₋₆alkyl, C₃₋₉cycloalkyl, C₂₋₆ alkenyl, C₂₋₆alkynyl, hydroxyl, ═O, halogen, —SH, ═S, —CF₃, —O—C₁₋₆alkyl, —OCF₃, —CHF₂; —OCHF₂, cyano, nitro, —C(O)OH, —NH₂, —NHC₁₋₆alkyl, and —N(C₁₋₆alkyl)₂

10. A compound, or an isomer (preferably a stereo-isomer or a tautomer), a solvate, a salt (preferably a pharmaceutically acceptable salt) or a prodrug thereof, preferably a pharmaceutically acceptable salt, solvate, hydrate, polymorph, tautomer, stereoisomer, or prodrug thereof, selected from one or more of the compounds of Table 1.

11. A pharmaceutical composition comprising a pharmaceutically acceptable carrier, and as active ingredient, an effective amount of a compound according to any one of claims 1 to 10.

12. The compound according to any one of claims 1 to 10, or a pharmaceutical composition according to claim 11, for use as a medicine.

13. The compound according to any one of claims 1 to 10, or a pharmaceutical composition according to claim 11, for use in the prevention or treatment of a YAP/TAZ-TEAD activation mediated disorder in an animal, mammal or human.

14. The compound according to claim 13, or a pharmaceutical composition according to claim 11, wherein the YAP/TAZ-TEAD activation mediated disorders is selected from the group comprising cancer, fibrosis and YAP/TAZ-TEAD activation mediated congenital disorders.

15. The compound according to claim 13, or a pharmaceutical composition according to claim 11, wherein the YAP/TAZ-TEAD activation mediated disorders is selected from lung cancer, breast cancer, head and neck cancer, oesophageal cancer, kidney cancer, bladder cancer, colon cancer, ovarian cancer, cervical cancer, endometrial cancer, liver cancer (including but not limited to cholangiocarcinoma), skin cancer, pancreatic cancer, gastric cancer, brain cancer and prostate cancer, mesotheliomas, and/or sarcomas.

16. The compound according to claim 13, or a pharmaceutical composition according to claim 11, wherein the YAP/TAZ-TEAD activation mediated disorders is selected from acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bronchogenic carcinoma, chondrosarcoma, chordoma, choriocarcinoma, chronic leukemia, chronic lymphocytic leukemia, chronic myelocytic (granulocytic) leukemia, chronic myelogenous leukemia, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, papillary adenocarcinomas, papillary carcinoma, pinealoma, polycythemia vera, rectal cancer, renal cell carcinoma, retinoblastoma, rhabdomyosarcoma, sarcoma, sebaceous gland carcinoma, seminoma, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

17. A method for the prevention or treatment of a YAP/TAZ-TEAD activation mediated disorders in an animal, mammal or human comprising administering to said animal, mammal or human in need for such prevention or treatment an effective dose of the compounds according to any one of claims 1 to 10.

18. A method of treatment or prevention of YAP/TAZ-TEAD activation mediated disorder according to claim 17 to a patient in need thereof in combination with one or more other medicines selected from EGFR inhibitors, MEK inhibitors, AXL inhibitors, B-RAF inhibitors, or RAS inhibitors.
